SEQUENCE LISTING (82 sequences)

SEQ ID NO: 1 - protein, 15 aa, Artificial Sequence
  Synthetic HPV E6 peptide
MHQKRTAMFQDPQER

SEQ ID NO: 2 - protein, 15 aa, Artificial Sequence
  Synthetic HPV E6 peptide
RTAMFQDPQERPRKL

SEQ ID NO: 3 - protein, 15 aa, Artificial Sequence
  Synthetic HPV E6 peptide
FQDPQERPRKLPQLC

SEQ ID NO: 4 - protein, 15 aa, Artificial Sequence
  Synthetic HPV E6 peptide
QERPRKLPQLCTELQ

SEQ ID NO: 5 - protein, 15 aa, Artificial Sequence
  Synthetic HPV E6 peptide
RKLPQLCTELQTTIH

SEQ ID NO: 6 - protein, 15 aa, Artificial Sequence
  Synthetic HPV E6 peptide
QLCTELQTTIHDIIL

SEQ ID NO: 7 - protein, 15 aa, Artificial Sequence
  Synthetic HPV E6 peptide
ELQTTIHDIILECVY

SEQ ID NO: 8 - protein, 15 aa, Artificial Sequence
  Synthetic HPV E6 peptide
LECVYCKQQLLRRII

SEQ ID NO: 9 - protein, 15 aa, Artificial Sequence
  Synthetic HPV E6 peptide
IILECVYCKQQLLRR

SEQ ID NO: 10 - protein, 15 aa, Artificial Sequence
  Synthetic HPV E6 peptide
CVYCKQQLLRREVYD

SEQ ID NO: 11 - protein, 15 aa, Artificial Sequence
  Synthetic HPV E6 peptide
KQQLLRREVYDFAFR

SEQ ID NO: 12 - protein, 15 aa, Artificial Sequence
  Synthetic HPV E6 peptide
LRREVYDFAFRDLCI

SEQ ID NO: 13 - protein, 15 aa, Artificial Sequence
  Synthetic HPV E6 peptide
VYDFAFRDLCIVYRD

SEQ ID NO: 14 - protein, 15 aa, Artificial Sequence
  Synthetic HPV E6 peptide
AFRDLCIVYRDGNPY

SEQ ID NO: 15 - protein, 15 aa, Artificial Sequence
  Synthetic HPV E6 peptide
LCIVYRDGNPYAVCD

SEQ ID NO: 16 - protein, 15 aa, Artificial Sequence
  Synthetic HPV E6 peptide
YRDGNPYAVCDKCLK

SEQ ID NO: 17 - protein, 15 aa, Artificial Sequence
  Synthetic HPV E6 peptide
NPYAVCDKCLKFYSK

SEQ ID NO: 18 - protein, 15 aa, Artificial Sequence
  Synthetic HPV E6 peptide
VCDKCLKFYSKISEY

SEQ ID NO: 19 - protein, 15 aa, Artificial Sequence
  Synthetic HPV E6 peptide
CLKFYSKISEYRHYC

SEQ ID NO: 20 - protein, 15 aa, Artificial Sequence
  Synthetic HPV E6 peptide
YSKISEYRHYCYSLY

SEQ ID NO: 21 - protein, 15 aa, Artificial Sequence
  Synthetic HPV E6 peptide
SEYRHYCYSLYGTTL

SEQ ID NO: 22 - protein, 15 aa, Artificial Sequence
  Synthetic HPV E6 peptide
HYCYSLYGTTLEQQY

SEQ ID NO: 23 - protein, 15 aa, Artificial Sequence
  Synthetic HPV E6 peptide
SLYGTTLEQQYNKPL

SEQ ID NO: 24 - protein, 15 aa, Artificial Sequence
  Synthetic HPV E6 peptide
TTLEQQYNKPLCDLL

SEQ ID NO: 25 - protein, 15 aa, Artificial Sequence
  Synthetic HPV E6 peptide
QQYNKPLCDLLIRCI

SEQ ID NO: 26 - protein, 15 aa, Artificial Sequence
  Synthetic HPV E6 peptide
KPLCDLLIRCINCQK

SEQ ID NO: 27 - protein, 15 aa, Artificial Sequence
  Synthetic HPV E6 peptide
DLLIRCINCQKPLCP

SEQ ID NO: 28 - protein, 15 aa, Artificial Sequence
  Synthetic HPV E6 peptide
RCINCQKPLCPEEKQ

SEQ ID NO: 29 - protein, 15 aa, Artificial Sequence
  Synthetic HPV E6 peptide
CQKPLCPEEKQRHLD

SEQ ID NO: 30 - protein, 15 aa, Artificial Sequence
  Synthetic HPV E6 peptide
LCPEEKQRHLDKKQR

SEQ ID NO: 31 - protein, 15 aa, Artificial Sequence
  Synthetic HPV E6 peptide
EKQRHLDKKQRFHNI

SEQ ID NO: 32 - protein, 15 aa, Artificial Sequence
  Synthetic HPV E6 peptide
HLDKKQRFHNIRGRW

SEQ ID NO: 33 - protein, 15 aa, Artificial Sequence
  Synthetic HPV E6 peptide
KQRFHNIRGRWTGRC

SEQ ID NO: 34 - protein, 15 aa, Artificial Sequence
  Synthetic HPV E6 peptide
HNIRGRWTGRCMSCC

SEQ ID NO: 35 - protein, 15 aa, Artificial Sequence
  Synthetic HPV E6 peptide
GRWTGRCMSCCRSSR

SEQ ID NO: 36 - protein, 15 aa, Artificial Sequence
  Synthetic HPV E6 peptide
GRCMSCCRSSRTRRE

SEQ ID NO: 37 - protein, 14 aa, Artificial Sequence
  Synthetic HPV E6 peptide
SCCRSSRTRRETQL

SEQ ID NO: 38 - protein, 15 aa, Artificial Sequence
  Synthetic HPV E7 peptide
MHGDTPTLHEYMLDL

SEQ ID NO: 39 - protein, 15 aa, Artificial Sequence
  Synthetic HPV E7 peptide
TPTLHEYMLDLQPET

SEQ ID NO: 40 - protein, 15 aa, Artificial Sequence
  Synthetic HPV E7 peptide
HEYMLDLQPETTDLY

SEQ ID NO: 41 - protein, 15 aa, Artificial Sequence
  Synthetic HPV E7 peptide
LDLQPETTDLYCYEQ

SEQ ID NO: 42 - protein, 15 aa, Artificial Sequence
  Synthetic HPV E7 peptide
PETTDLYCYEQLNDS

SEQ ID NO: 43 - protein, 15 aa, Artificial Sequence
  Synthetic HPV E7 peptide
DLYCYEQLNDSSEEE

SEQ ID NO: 44 - protein, 15 aa, Artificial Sequence
  Synthetic HPV E7 peptide
YEQLNDSSEEEDEID

SEQ ID NO: 45 - protein, 15 aa, Artificial Sequence
  Synthetic HPV E7 peptide
NDSSEEEDEIDGPAG

SEQ ID NO: 46 - protein, 15 aa, Artificial Sequence
  Synthetic HPV E7 peptide
EEEDEIDGPAGQAEP

SEQ ID NO: 47 - protein, 15 aa, Artificial Sequence
  Synthetic HPV E7 peptide
EIDGPAGQAEPDRAH

SEQ ID NO: 48 - protein, 15 aa, Artificial Sequence
  Synthetic HPV E7 peptide
PAGQAEPDRAHYNIV

SEQ ID NO: 49 - protein, 15 aa, Artificial Sequence
  Synthetic HPV E7 peptide
AEPDRAHYNIVTFCC

SEQ ID NO: 50 - protein, 15 aa, Artificial Sequence
  Synthetic HPV E7 peptide
RAHYNIVTFCCKCDS

SEQ ID NO: 51 - protein, 15 aa, Artificial Sequence
  Synthetic HPV E7 peptide
NIVTFCCKCDSTLRL

SEQ ID NO: 52 - protein, 15 aa, Artificial Sequence
  Synthetic HPV E7 peptide
FCCKCDSTLRLCVQS

SEQ ID NO: 53 - protein, 15 aa, Artificial Sequence
  Synthetic HPV E7 peptide
CDSTLRLCVQSTHVD

SEQ ID NO: 54 - protein, 15 aa, Artificial Sequence
  Synthetic HPV E7 peptide
LRLCVQSTHVDIRTL

SEQ ID NO: 55 - protein, 15 aa, Artificial Sequence
  Synthetic HPV E7 peptide
VQSTHVDIRTLEDLL

SEQ ID NO: 56 - protein, 15 aa, Artificial Sequence
  Synthetic HPV E7 peptide
HVDIRTLEDLLMGTL

SEQ ID NO: 57 - protein, 15 aa, Artificial Sequence
  Synthetic HPV E7 peptide
RTLEDLLMGTLGIVC

SEQ ID NO: 58 - protein, 15 aa, Artificial Sequence
  Synthetic HPV E7 peptide
DLLMGTLGIVCPICS

SEQ ID NO: 59 - protein, 14 aa, Artificial Sequence
  Synthetic HPV E7 peptide
GTLGIVCPICSQKP

SEQ ID NO: 60 - DNA, 92 bp, Artificial Sequence
  Synthetic 5 UTR
tcaagcttttggaccctcgtacagaagctaatacgactcactatagggaaataagagagaaaagaagagtaagaagaaatataagagccacc

SEQ ID NO: 61 - DNA, 119 bp, Artificial Sequence
  Synthetic 3 UTR
tgataataggctggagcctcggtggccatgcttcttgccccttgggcctccccccagcccctcctccccttcctgcacccgtacccccgtggtctttgaataaagtctgagtgggcggc

SEQ ID NO: 62 - DNA, 164 bp, Artificial Sequence
  Synthetic 3 UTR with miR-122 and miR-142-3p sites
tgataataggctggagcctcggtggccatgcttcttgccccttgggcccaaacaccattgtcacactccatccccccagcccctcctccccttcctccataaagtaggaaacactacatgcacccgtacccccgtggtctttgaataaagtctgagtgggcggc

SEQ ID NO: 63 - protein, 22 aa, Artificial Sequence
  Synthetic 2A peptide amino acid sequence
GSGATNFSLLKQAGDVEENPGP

SEQ ID NO: 64 - DNA, 66 bp, Artificial Sequence
  Synthetic Nucleotide sequence encoding 2A peptide
ggaagcggagctactaacttcagcctgctgaagcaggctggagacgtggaggagaaccctggacct

SEQ ID NO: 65 - DNA, 108 bp, Artificial Sequence
  Synthetic Nucleotide sequence encoding 2A peptide
tccggactcagatccggggatctcaaaattgtcgctcctgtcaaacaaactcttaactttgatttactcaaactggctggggatgtagaaagcaatccaggtccactc

SEQ ID NO: 66 - RNA, 87 nt, Artificial Sequence
  Synthetic miR-142
gacagugcagucacccauaaaguagaaagcacuacuaacagcacuggaggguguaguguuuccuacuuuauggaugaguguacugug

SEQ ID NO: 67 - RNA, 23 nt, Artificial Sequence
  Synthetic miR-142-3p
uguaguguuuccuacuuuaugga

SEQ ID NO: 68 - RNA, 23 nt, Artificial Sequence
  Synthetic miR-142-3p binding site
uccauaaaguaggaaacacuaca

SEQ ID NO: 69 - RNA, 21 nt, Artificial Sequence
  Synthetic miR-142-5p
cauaaaguagaaagcacuacu

SEQ ID NO: 70 - RNA, 21 nt, Artificial Sequence
  Synthetic miR-142-5p binding site
aguagugcuuucuacuuuaug

SEQ ID NO: 71 - RNA, 85 nt, Artificial Sequence
  Synthetic miR-122
ccuuagcagagcuguggagugugacaaugguguuugugucuaaacuaucaaacgccauuaucacacuaaauagcuacugcuaggc

SEQ ID NO: 72 - RNA, 22 nt, Artificial Sequence
  Synthetic miR-122-3p
aacgccauuaucacacuaaaua

SEQ ID NO: 73 - RNA, 22 nt, Artificial Sequence
  Synthetic miR-122-3p binding site
uauuuagugugauaauggcguu

SEQ ID NO: 74 - RNA, 22 nt, Artificial Sequence
  Synthetic miR-122-5p
uggagugugacaaugguguuug

SEQ ID NO: 75 - RNA, 22 nt, Artificial Sequence
  Synthetic miR-122-5p binding site
caaacaccauugucacacucca

SEQ ID NO: 76 - DNA, 47 bp, Artificial Sequence
  Synthetic Standard 5 UTR sequence
gggaaataagagagaaaagaagagtaagaagaaatataagagccacc

SEQ ID NO: 77 - DNA, 41 bp, Artificial Sequence
  Synthetic 5 UTR sequence
gggaaataagagagaaaagaagagtaagaagaaatataaga

SEQ ID NO: 78 - DNA, 57 bp, Artificial Sequence
  Synthetic V1-UTR sequence
gggaaataagagagaaaagaagagtaagaagaaatataagaccccggcgccgccacc

SEQ ID NO: 79 - DNA, 54 bp, Artificial Sequence
  Synthetic V2-UTR sequence
gggaaataagagagaaaagaagagtaagaagaaatataagaccccggcgccacc

SEQ ID NO: 80 - DNA, 10 bp, Artificial Sequence
  Synthetic V1 GC-rich RNA element
ccccggcgcc

SEQ ID NO: 81 - DNA, 12 bp, Artificial Sequence
  Synthetic GC-rich element wherein CCGn, where n=4
ccgccgccgccg

SEQ ID NO: 82 - DNA, 15 bp, Artificial Sequence
  Synthetic GC-rich element wherein CCGn, where n=5
ccgccgccgccgccg

(19) United States
(12) Patent Application Publication
(10) Pub. No.: US 2019/0314291 A1
(43) Pub. Date: Oct. 17, 2019

(54) COMPOSITIONS AND METHODS FOR DELIVERY OF AGENTS TO IMMUNE CELLS

(71) Applicant: ModernaTX, Inc., Cambridge, MA (US)

(72) Inventors: Gilles BESIN, Medford, MA (US); Luis BRITO, Concord, MA (US); Stephen G. HOGE, Brookline, MA (US); Edward HENNESSY, Cambridge, MA (US); Mark CORNEBISE, Cambridge, MA (US); Kerry BENENATO, Sudbury, MA (US); Staci SABNIS, Cambridge, MA (US); Michael W. DANNEMAN, Belmont, MA (US)

(21) Appl. No.: 16/262,758

(22) Filed: Jan. 30, 2019

Related U.S. Application Data
(60) Provisional application No. 62/744,825, filed on Oct. 12, 2018.
(60) Provisional application No. 62/733,609, filed on Sep. 19, 2018.
(60) Provisional application No. 62/623,922, filed on Jan. 30, 2018.

(51) Int. Cl. A61K 9/51; C07K 14/525; A61P 35/00; C07K 14/705; A61K 39/245

(52) U.S. Cl. CPC A61K 9/5123; C07K 14/525; A61P 35/00; C12N 2710/16134; C07K 14/70503; A61K 39/245; A61K 9/5146

(57) ABSTRACT

The disclosure features immune cell delivery lipid nanoparticle (LNP) compositions that allow for enhanced delivery of agents, e.g., nucleic acids, such as therapeutic and/or prophylactic RNAs, to immune cells, in particular T cells, as well as B cells, dendritic cells and monocytes. The LNPs comprise an effective amount of an immune cell delivery potentiating lipid such that delivery of an agent by an immune cell delivery LNP is enhanced as compared to an LNP lacking the immune cell delivery potentiating agent. Methods of using the immune cell delivery LNPs for delivery of agents, e.g., nucleic acid delivery, for protein expression, for modulating immune cell activity and modulating immune responses are also disclosed.

### RELATED APPLICATIONS

This application claims the benefit of U.S. Provisional Application Ser. No. 62/623,922, filed on Jan. 30, 2018, U.S. Provisional Application Ser. No. 62/733,609, filed on Sep. 19, 2018, and U.S. Provisional Application No. 62/744,825, filed on Oct. 12, 2018. The entire contents of the above-referenced applications is incorporated herein by this reference.

### BACKGROUND OF THE DISCLOSURE

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutics and/or prophylactics such as nucleic acids to cells.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more “cationic” and/or amino (ionizable) lipids, phospholipids and/or polyunsaturated lipids (helper lipids), structural lipids (e.g., sterols), and/or lipids containing polyethylene glycol (PEG lipids). Optimally, lipid nanoparticle compositions contain each of i) an amino (ionizable) lipid, 2) a phospholipid, 3) a structural lipid or blend thereof, 4) a PEG lipid and 5) an agent. Cationic and/or ionizable lipids include, for example, amine-containing lipids that can be readily protonated. Though a variety of such lipid-containing nanoparticle compositions have been demonstrated, effective delivery vehicles for reaching desired cell populations while maintaining safety, and efficacy, are still lacking.

Clinical studies have shown that stimulation of a patient's immune system can provide effective treatment against cancer. On the other hand, systemic immune stimulation is often associated with autoimmune-type pathologies and/or toxicities. The ability to effectively modulate immune responses by controlled delivery of immunomodulatory agents to immune cell populations would improve both safety and efficacy beyond that offered by current treatments. However, efforts to modulate the immune system have been hampered by the lack of delivery systems that are capable of reliably delivering biologically active substances such as small molecule drugs, proteins, and nucleic acids to immune cells, which have been notoriously difficult to transfect. Safe, efficient delivery of nucleic acid molecules to immune cells in vivo also remains elusive. Moreover, current technologies for immune modulation, which employ soluble proteins (e.g., cytokines and antibodies) are not effective for inducing expression of intracellular or transmembrane proteins or for reducing expression of endogenous proteins. The ability to effectively deliver nucleic acid molecules to immune cells, in vitro or in vivo, would make new immune therapies possible.

### SUMMARY OF THE DISCLOSURE

Provided herein are improved lipid-based compositions, immune cell delivery lipid nanoparticles (LNPs), that enhance delivery of an agent to immune cells, such as lymphocytes or myeloid cells. In one embodiment, the immune cell is a T cell (e.g., CD4+ and/or CD8+ T cells, naïve cells, effector cells, and/or memory cells), a dendritic cell, a macrophage, a monocyte, an NK cell (including immature and activated NK cells), an NK T cell, and/or a B cell (including plasma cells), and their uses thereof. Preferably, the immune cell is a human or primate immune cell.

In some aspects, by using an immune cell delivery LNP, delivery to an immune cell is enhanced in vitro, while in other aspects, delivery to an immune cell is enhanced in vivo. When administered in vivo, in one embodiment, immune cell delivery LNPs demonstrate enhanced delivery of agents to the spleen and bone marrow when compared to control LNPs. In some aspects, the immune cell, e.g., a human immune cell, is contacted with the LNP in vitro. In some aspects, the immune cell is contacted with the LNP in vivo by administering the LNP to a subject, e.g., a human subject. In one embodiment, the subject is one that would benefit from modulation of protein expression or activity in an immune cell. In some aspects, the LNP is administered intravenously. In some aspects, the LNP is administered intramuscularly. In some aspects, the LNP is administered by a route selected from the group consisting of subcutaneously, intranodally and intratumorally.

The agent to be delivered by an immune cell delivery LNP may comprise a molecule which it would be of benefit to deliver to an immune cell. In one embodiment, the agent may comprise or consist of a nucleic acid molecule. In some aspects, the nucleic acid molecule is selected from the group consisting of RNA, mRNA, RNAi, dsRNA, siRNA, antisense RNA, ribozyme, CRISPR/Cas9, ssDNA and DNA. In some aspects, the nucleic acid molecule is RNA selected from the group consisting of a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof. In some embodiments, the nucleic acid molecule is an siRNA molecule. In some embodiments, the nucleic acid molecule is a miR. In some embodiments, the nucleic acid molecule is an antagomir. In some aspects, the nucleic acid molecule is DNA. In some aspects, the nucleic acid molecule is mRNA.

Accordingly, in one aspect, the disclosure pertains to an immune cell delivery lipid nanoparticle comprising:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell.

In another embodiment, the disclosure pertains to an immune cell delivery lipid nanoparticle comprising:
(i) an ionizable lipid;
(ii) a sterol or other structural lipid;
(iii) a non-cationic helper lipid or phospholipid;
(iv) a PEG-lipid, and
(v) an agent comprising an mRNA encoding a protein of interest,

wherein one or more of (i) the ionizable lipid or (ii) the sterol or other structural lipid comprises an immune cell delivery potentiating lipid in an amount effective to enhance delivery of the lipid nanoparticle to an immune cell.

In another embodiment, the disclosure pertains to an immune cell delivery lipid nanoparticle comprising:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q and/or promotes (e.g., increases, stimulates, enhances) the binding of the LNP to C1q, as compared to a control lipid nanoparticle lacking the C1q binding lipid.

In another aspect, the disclosure pertains to a method of delivering an agent to an immune cell, the method comprising contacting the immune cell with an immune cell delivery lipid nanoparticle comprising:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell, such that the agent is delivered to the immune cell.

In another aspect, the disclosure pertains to a method of inducing expression of a protein of interest in or on an immune cell, the method comprising contacting the immune cell with an immune cell delivery lipid nanoparticle comprising:
(i) an ionizable lipid;
(ii) a sterol or other structural lipid;
(iii) a non-cationic helper lipid or phospholipid;
(iv) an agent comprising a nucleic acid encoding the protein of interest, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell, such that expression of the protein of interest is induced in or on the immune cell.

In another aspect, the disclosure pertains to a method of modulating T cell activation or activity, the method comprising contacting a T cell with an immune cell delivery lipid nanoparticle comprising:
(i) an ionizable lipid;
(ii) a sterol or other structural lipid;
(iii) a non-cationic helper lipid or phospholipid;
(iv) an agent comprising a nucleic acid that modulates T cell activation or activity, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to a T cell, such that T cell activation or activity is modulated.

In another aspect, the disclosure pertains to a method of increasing an immune response to a protein, the method comprising contacting immune cells with an immune cell delivery lipid nanoparticle comprising:
(i) an ionizable lipid;
(ii) a sterol or other structural lipid;
(iii) a non-cationic helper lipid or phospholipid;
(iv) an agent comprising a nucleic acid molecule, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells, such that the immune response to the protein is increased.

In another aspect, the disclosure pertains to a method of increasing a T cell response to a cancer antigen, the method comprising contacting the T cell with an immune cell delivery lipid nanoparticle comprising:
(i) an ionizable lipid;
(ii) a sterol or other structural lipid;
(iii) a non-cationic helper lipid or phospholipid;
(iv) an agent comprising an mRNA encoding a chimeric antigen receptor (CAR) that binds the cancer antigen, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells, such that the T cell response to the cancer antigen is increased.

In another aspect, the disclosure pertains to a method of enhancing an immune response to an antigen of interest in a subject, the method comprising administering to the subject an immune cell delivery lipid nanoparticle comprising:
(i) an ionizable lipid;
(ii) a sterol or other structural lipid;
(iii) a non-cationic helper lipid or phospholipid;
(iv) an agent comprising an mRNA encoding an antigen of interest, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells, such that the immune response to the antigen of interest is enhanced in the subject, as compared to the immune response to the antigen of interest induced by an LNP encapsulating the mRNA encoding the antigen of interest but lacking the immune cell delivery potentiating lipid.

In another aspect, the disclosure pertains to a method of modulating B cell activation or activity, the method comprising contacting a B cell with an immune cell delivery lipid nanoparticle comprising:
(i) an ionizable lipid;
(ii) a sterol or other structural lipid;
(iii) a non-cationic helper lipid or phospholipid;
(iv) an agent comprising a nucleic acid molecule, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells, such that B cell activation or activity is modulated.

In some aspects, the method further comprises administering, concurrently or consecutively, a second LNP encapsulating the same or different nucleic acid molecule, wherein the second LNP lacks an immune cell delivery potentiating lipid. In other aspects, the method further comprises administering, concurrently or consecutively, a second LNP encapsulating a different nucleic acid molecule, wherein the second LNP comprises an immune cell delivery potentiating lipid.

In one embodiment of the LNPs or methods of the disclosure, the enhanced delivery is relative to a lipid nanoparticle lacking the immune cell delivery potentiating lipid. In another embodiment of the LNPs or methods of the disclosure, the enhanced delivery is relative to a suitable control.

In one embodiment of the LNPs or methods of the disclosure, the agent stimulates protein expression in the immune cell. In another embodiment of the LNPs or methods of the disclosure, the agent inhibits protein expression in the immune cell. In another embodiment of the LNPs or methods of the disclosure, the agent encodes a soluble protein that modulates immune cell activity. In another embodiment of the LNPs or methods of the disclosure, the agent encodes an intracellular protein that modulates immune cell activity. In another embodiment of the LNPs or methods of the disclosure, the agent encodes a transmembrane protein that modulates immune cell activity. In another embodiment of the LNPs or methods of the disclosure, the agent enhances immune function. In another embodiment of the LNPs or methods of the disclosure, the agent inhibits immune function.

In one embodiment of the LNPs or methods of the disclosure, the immune cell is a T cell. In another embodiment of the LNPs or methods of the disclosure, the immune cell is a B cell. In another embodiment of the LNPs or methods of the disclosure, the immune cell is a dendritic cell or a myeloid cell.

In one embodiment of the LNPs or methods of the disclosure, the LNP comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In one embodiment, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In one embodiment, the phytosterol is a combination of Compound S-141, Compound S-140, Compound S-143 and Compound S-148. In one embodiment, the phytosterol comprises a sitosterol or a salt or an ester thereof. In one embodiment, the phytosterol comprises a stigmasterol or a salt or an ester thereof. In one embodiment, the phytosterol is beta-sitosterol

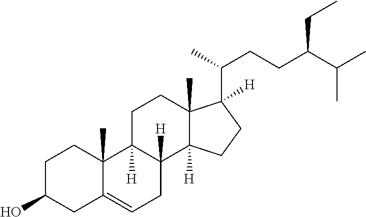

or a salt or an ester thereof.

In one embodiment of the LNPs or methods of the disclosures, the LNP comprises a phytosterol, or a salt or ester thereof, and cholesterol or a salt thereof.

In some embodiments, the immune cell is a T cell and the phytosterol or a salt or ester thereof is selected from the group consisting of β-sitosterol, β-sitostanol, campesterol, and brassicasterol, and combinations thereof. In one embodiment, the phytosterol is β-sitosterol. In one embodiment, the phytosterol is β-sitostanol. In one embodiment, the phytosterol is campesterol. In one embodiment, the phytosterol is brassicasterol.

In some embodiments, the immune cell is a monocyte or a myeloid cell and the phytosterol or a salt or ester thereof is selected from the group consisting of β-sitosterol, and stigmasterol, and combinations thereof. In one embodiment, the phytosterol is β-sitosterol. In one embodiment, the phytosterol is stigmasterol.

In some embodiments of the LNPs or methods of the disclosure, the LNP comprises a sterol, or a salt or ester thereof, and cholesterol or a salt thereof, wherein the immune cell is a monocyte or a myeloid cell and the sterol or a salt or ester thereof is selected from the group consisting of β-sitosterol-d7, brassicasterol, Compound S-30, Compound S-31 and Compound S-32.

In one embodiment, the mol % cholesterol is between about 1% and 50% of the mol % of phytosterol present in the lipid nanoparticle. In one embodiment, the mol % cholesterol is between about 10% and 40% of the mol % of phytosterol present in the lipid nanoparticle. In one embodiment, the mol % cholesterol is between about 20% and 30% of the mol % of phytosterol present in the lipid nanoparticle. In one embodiment, the mol % cholesterol is about 30% of the mol % of phytosterol present in the lipid nanoparticle.

In one embodiment of the LNPs or methods of the disclosure, the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or comprises a compound selected from the group consisting of: Compound X (also referred to as Compound I-18), Compound Y (also referred to as Compound I-25), Compound I-48, Compound I-50, Compound I-109, Compound I-111, Compound I-113, Compound I-181, Compound I-182, Compound I-244, Compound I-292, Compound I-301, Compound I-321, Compound I-322, Compound I-326, Compound I-328, Compound I-330, Compound I-331, Compound I-332 or Compound I-M. In one embodiment, the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M. In one embodiment, the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181. In one embodiment, the ionizable lipid comprises a compound selected from the group consisting of Compound X, Compound Y, Compound I-309, Compound I-317, Compound I-321, Compound I-292, Compound I-326, Compound I-347, Compound I-348, Compound I-349, Compound I—350, Compound I-351, Compound I-352, Compound I-182, Compound I-301, Compound I-48, Compound I-50, Compound I-328, Compound I-330, Compound I-109, Compound I-111 and Compound I-181. In one embodiment, the ionizable lipid comprises a compound selected from the group consisting of Compound I-309, Compound I-317, Compound I-347, Compound I-348, Compound I-349, Compound I-350, Compound I-351 and Compound I-352.

In some embodiments, the immune cell is a T cell and the ionizable lipid comprises a compound selected from the group consisting of Compound I-301, Compound I-321 and Compound I-326. In other embodiments, the immune cell is a monocyte or a myeloid cell and the ionizable lipid comprises a compound selected from the group consisting of Compound X, Compound I-109, Compound I-111, Compound I-181, Compound I-182, and Compound I-244

In any of the foregoing or related aspects, the ionizable lipid of the LNP of the disclosure comprises at least one compound selected from the group consisting of: Compound I 18 (also referred to as Compound X), Compound I-25 (also referred to as Compound Y), Compound I-48, Compound I-50, Compound I-109, Compound I-111, Compound I-113, Compound I-181, Compound I-182, Compound I-244, Compound I-292, Compound I-301, Compound I-321, Compound I-322, Compound I-326, Compound I-328, Compound I-330, Compound I-331, and Compound I-332. In another embodiment, the ionizable lipid of the LNP of the disclosure comprises a compound selected from the group consisting of: Compound I 18 (also referred to as Compound X), Compound I-25 (also referred to as Compound Y), Compound I-48, Compound I-50, Compound I-109, Compound I-111, Compound I-181, Compound I-182, Compound I-292, Compound I-301, Compound I-321, Compound I-326, Compound I-328, and Compound I-330. In another embodiment, the ionizable lipid of the LNP of the disclosure comprises a compound selected from the group consisting of: Compound I-182, Compound I-301, Compound I-321, and Compound I-326.

In one embodiment of the LNPs or methods of the disclosure, the non-cationic helper lipid or phospholipid comprises a compound selected from the group consisting of DSPC, DMPE, DOPC and Compound H-409. In one embodiment of the LNPs or methods of the disclosure, the non-cationic helper lipid or phospholipid comprises a compound selected from the group consisting of DSPC, DPPC, DMPE, DMPC, DOPC, Compound H-409, Compound H-418, Compound H-420, Compound H-421 and Compound H-422. In one embodiment, the phospholipid is DSPC. In one embodiment of the LNPs or methods of the disclosure, the non-cationic helper lipid or phospholipid comprises a compound selected from the group consisting of DPPC, DMPC, Compound H-418, Compound H-420, Compound H-421 and Compound H-422.

In one embodiment of the LNPs or methods of the disclosure, the immune cell is a T cell and the non-cationic helper lipid or phospholipid comprises a compound selected from the group consisting of DSPC, DMPE, and Compound H-409. In one embodiment, the phospholipid is DSPC. In one embodiment, the phospholipid is DMPE. In one embodiment, the phospholipid is Compound H-409.

In one embodiment of the LNPs or methods of the disclosure, the immune cell is a monocyte or a myeloid cell and the non-cationic helper lipid or phospholipid comprises a compound selected from the group consisting of DOPC, DMPE, and Compound H-409. In one embodiment, the phospholipid is DSPC. In one embodiment, the phospholipid is DMPE. In one embodiment, the phospholipid is Compound H-409.

In one embodiment of the LNPs or methods of the disclosure, the LNP comprises a PEG-lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. In one embodiment, the PEG lipid is selected from the group consisting of Compound 428, Compound P-L16, Compound P-L17, Compound P-L18, Compound P-L19, Compound P-L1, and Compound P-L2. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In one embodiment, the PEG lipid is selected from the group consisting of Compound P-L3, Compound P-L4, Compound P-L6, Compound P-L8, Compound P-L9 and Compound P-L25.

In one embodiment of the LNPs or methods of the disclosure, the LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid. In one embodiment, of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment, of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In one embodiment of the LNPs or methods of the disclosure, the LNP comprises:

i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

In some aspects, the disclosure provides a lipid nanoparticle (LNP) for use in a method of immune therapy with enhanced delivery to an immune cell,

wherein the LNP comprises
(i) a sterol or other structural lipid;
(ii) an ionizable lipid; and
(iii) an agent for delivery to an immune cell;

wherein one or more of (i) the sterol or other structural lipid and/or (ii) the ionizable lipid comprises an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

In some aspects, the disclosure provides a lipid nanoparticle (LNP) for use in a method of immune therapy with enhanced delivery to an immune cell,

wherein the LNP comprises
(i) a sterol or other structural lipid;
(ii) an ionizable lipid; and
(iii) an agent for delivery to an immune cell;

wherein the sterol or other structural lipid comprises an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

In some aspects, the disclosure provides a lipid nanoparticle (LNP) for use in a method of immune therapy with enhanced delivery to an immune cell,

wherein the LNP comprises
(i) a sterol or other structural lipid;
(ii) an ionizable lipid; and
(iii) an agent for delivery to an immune cell;

the ionizable lipid comprises an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

In some aspects, the disclosure provides a lipid nanoparticle (LNP) for use in a method of immune therapy with enhanced delivery to an immune cell,

wherein the LNP comprises
(i) a sterol or other structural lipid;
(ii) an ionizable lipid; and
(iii) an agent for delivery to an immune cell;

wherein (i) the sterol or other structural lipid and (ii) the ionizable lipid comprise an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

In any of the foregoing or related aspects, the sterol or other structural lipid is a phytosterol or cholesterol.

In any of the foregoing or related aspects, the immune cell delivery potentiating lipid binds to C1q and/or promotes the binding of the LNP comprising said lipid to C1q compared to a control LNP lacking the immune cell delivery potentiating lipid and/or increases uptake of C1q-bound LNP into an immune cell compared to a control LNP lacking the immune cell delivery potentiating lipid.

In any of the foregoing or related aspects, the agent for delivery to an immune cell is a nucleic acid molecule. In some aspects, the agent stimulates expression of a protein of interest in the immune cell. In some aspects, the agent for delivery to an immune cell is a nucleic acid molecule encoding a protein of interest. In some aspects, the agent for delivery to an immune cell is an mRNA encoding a protein of interest.

In any of the foregoing or related aspects, the expression of the protein of interest in the immune cell is enhanced relative to a control LNP lacking the immune cell delivery potentiating lipid. In some aspects, the agent encodes a protein that modulates immune cell activity.

In any of the foregoing or related aspects, the immune cell is a lymphocyte. In some aspects, the immune cell is a T cell or a B cell. In some aspects, the immune cell is a T cell. In some aspects, the immune cell is a B cell. In some aspects, the immune cell is a dendritic cell or a myeloid cell. In some aspects, the immune cell is a dendritic cell. In some aspects, the immune cell is a myeloid cell.

In any of the foregoing or related aspects, the lipid nanoparticle further comprises

(iv) a non-cationic helper lipid or phospholipid, and/or

(v) a PEG-lipid.

In some aspects, the lipid nanoparticle further comprises a non-cationic helper lipid or phospholipid. In some aspects, the nanoparticle further comprise a PEG-lipid. In some aspects, the lipid nanoparticle further comprises a non-cationic helper lipid or phospholipid, and a PEG-lipid.

In any of the foregoing or related aspects, the methods described herein result in modulation of activation or activity of an immune cell. In some aspects, the methods result in modulation of activation or activity of a T cell or a B cell.

In any of the foregoing or related aspects, the methods described herein result in an increase of an immune response to an antigen of interest, optionally in an increase of a T-cell response to a cancer antigen. In some aspects, the methods described herein result in an increase of an immune response to an antigen of interest. In some aspects, the methods described herein result in an increase of a T-cell response to a cancer antigen.

In some aspects, the disclosure provides an in vitro method of delivering an agent to an immune cell, the method comprising contacting the immune cell with a lipid nanoparticle comprising an immune cell delivery potentiating lipid. In some aspects of the in vitro method, the method results in
a. modulation of activation or activity of an immune cell, e.g., of a T cell or of a B cell; and/or
b. an increase of an immune response to an antigen of interest, optionally an increase of a T-cell response to a cancer antigen.

In some aspects of the in vitro method, the method results in modulation of activation or activity of an immune cell. In some aspects of the in vitro method, the method results in an increase of an immune response to an antigen of interest. In some aspects of the in vitro method, the method results in an increase of a T-cell response to a cancer antigen. In some aspects of the in vitro method, the method results in
a. modulation of activation or activity of an immune cell; and
b. an increase of an immune response to an antigen of interest.

In some aspects of the in vitro method, the method results in
a. modulation of activation or activity of an immune cell; and
b. an increase of a T-cell response to a cancer antigen.

### BRIEF DESCRIPTION OF THE DRAWINGS

FIGS. 1A-1B are bar graphs showing results of incubating human AML cells ex vivo with an mOX40L-encoding mRNA encapsulated in an LNP containing either Compound X/cholesterol/DSPC/PEG DMG (LNP 1), Compound X/cholesterol/DSPC/Compound 428 (LNP 2) or Compound X/beta-sitosterol/cholesterol/PEG DMG (LNP 3). PBS was used as a negative control. FIG. 1A shows percentage of mOX40L+ cells determined by flow cytometry. FIG. 1B shows PE intensity per cell as determined by fluorescence microscopy.

FIGS. 2A-2D show flow cytometry graphs of spleen cells from PDX mice reconstituted with AML PBMCs treated intravenously with either PBS (FIG. 2A), mOX40L-encoding mRNA encapsulated in an LNP containing Compound X-cholesterol-DSPC-PEG DMG (LNP 1; FIG. 2B), mOX40L-encoding mRNA encapsulated in an LNP containing Compound X-cholesterol-DSPC-Compound 428 (LNP 2; FIG. 2C) or mOX40L-encoding mRNA encapsulated in an LNP containing Compound X-beta-sitosterol/cholesterol-DSPC-PEG DMG (LNP 3; FIG. 2D), showing T cell transfection. The X-axis for each graph shows hCD3+ cells. The Y-axis for each graph shows mOX40L+ cells. For each treatment, the four graphs (left to right) represent results from four different mice.

FIGS. 3A-3B show flow cytometry graphs of human PBMCs (Donor 1) incubated ex vivo with either 20 ng (FIG. 3A) or 50 ng (FIG. 3B) of mOX40L-encoding mRNA encapsulated in either an LNP containing (left to right) Compound X-cholesterol-DSPC-PEG DMG (LNP 1), an LNP containing Compound X-cholesterol-DSPC-Compound 428 (LNP 2) or an LNP containing Compound X-beta-sitosterol/cholesterol-DSPC-PEG DMG (LNP 3), showing T cell transfection. PBS was used as a negative control (left-most panels). The X-axis for each graph shows mOX40L+ cells. The Y-axis for each graph shows hCD3+ cells.

FIGS. 4A-4B show flow cytometry graphs of human PBMCs (Donor 2) incubated ex vivo with either 20 ng (FIG. 4A) or 50 ng (FIG. 4B) of mOX40L-encoding mRNA encapsulated in either an LNP containing (left to right) Compound X-cholesterol-DSPC-PEG DMG (LNP 1), an LNP containing Compound X-cholesterol-DSPC-Compound 428 (LNP 2) or an LNP containing Compound X-beta-sitosterol/cholesterol-DSPC-PEG DMG (LNP 3), showing T cell transfection. PBS was used as a negative control (left-most panels). The X-axis for each graph shows mOX40L+ cells. The Y-axis for each graph shows hCD3+ cells.

FIGS. 5A-5D show flow cytometry graphs of human PBMCs incubated ex vivo with either PBS or one of three different lots of an LNP containing Compound X-beta-sitosterol/cholesterol-DSPC-PEG DMG encapsulating an mOX40L-encoding mRNA (panels left to right), showing T cell transfection. FIGS. 5A-5D represent four different technical replicates. The X-axis for each graph shows mOX40L+ cells. The Y-axis for each graph shows hCD3+ cells.

FIGS. 6A-6B show flow cytometry graphs of human PBMCs incubated ex vivo with either PBS (FIG. 6A) or an LNP containing Compound X-beta-sitosterol/cholesterol-DSPC-PEG DMG encapsulating an EGFP-encoding mRNA (FIG. 6B), showing T cell transfection. The panels (left to right) represent four different replicates and an overlay composite. The X-axis for each graph shows EGFP+ cells. The Y-axis for each graph shows hCD3+ cells.

FIG. 7 shows flow cytometry graphs of mouse PBMCs incubated ex vivo with 50 ng of mOX40L-encoding mRNA encapsulated in either an LNP containing (left to right) Compound X-cholesterol-DSPC-PEG DMG (LNP 1), an LNP containing Compound X-cholesterol-DSPC-Compound 428 (LNP 2) or an LNP containing Compound X-beta-sitosterol/cholesterol-DSPC-PEG DMG (LNP 3), showing lack of mouse T cell transfection. PBS was used as a negative control (left-most panels). The X-axis for each graph shows mOX40L+ cells. The Y-axis for each graph shows hCD3+ cells.

FIGS. 8A-8D show flow cytometry graphs of human PBMCs incubated ex vivo with mRNA encapsulated in an LNP containing Compound X-beta-sitosterol/cholesterol-DSPC-PEG DMG, following by cell sorting for the cell types indicated at the top. FIGS. 8A-8D show results for Donors 1-4, respectively. The percentage of cells transfected is shown at the bottom for each cell type examined.

FIGS. 9A-9C show flow cytometry graphs of human PBMCs incubated ex vivo with mRNA encapsulated in an LNP containing Compound X-beta-sitosterol/cholesterol-DSPC-PEG DMG (LNP 3), following by cell sorting for T cell subsets. FIG. 9A show results for CD4+ T cells. FIG. 9B shows the results for CD8+ T cells. FIG. 9C shows the results for CD4+CD25+CD127^{low} Treg cells.

FIG. 10 is a bar graph showing the percentage of mOX40L+ cells in splenic cells from non-human primates treated in vivo with intravenous injection of an mOX40L-encoding mRNA encapsulated in an LNP containing either Compound X/PEG DMG and cholesterol (LNP 1), Compound X/Compound 428 and cholesterol (LNP 2), Compound X/DSPE PEG and cholesterol (LNP 4) or Compound X/beta-sitosterol/cholesterol/PEG DMG (LNP 3).

FIGS. 11A-11C show flow cytometry graphs of spleen cells from non-human primates treated in vivo by intravenous injection of the LNP compositions indicated at the top of the panels (left to right) Compound X/PEG DMG and cholesterol (LNP 1), Compound X/Compound 428 and cholesterol (LNP 2), Compound X/DSPE PEG and cholesterol (LNP 4) or Compound X/beta-sitosterol/cholesterol/PEG DMG (LNP 3), encapsulating an mOX40L-encoding mRNA, showing splenic T cell transfection. FIGS. 11A and 11B show results from two different animals. FIG. 11C shows composite overlay results from the two animals. The X-axis for each graph shows mOX40L+ cells. The Y-axis for each graph shows cyno CD3+ cells.

FIGS. 12A-12C show flow cytometry graphs of spleen cells from non-human primates treated in vivo with intravenous injection of the LNP compositions indicated at the top of the panels (left to right) Compound X/PEG DMG and cholesterol (LNP 1), Compound X/Compound 428 and cholesterol (LNP 2), Compound X/DSPE PEG and cholesterol (LNP 4) or Compound X/beta-sitosterol/cholesterol/PEG DMG (LNP 3), encapsulating an mOX40L-encoding mRNA, showing splenic B cell transfection. FIGS. 12A and 12B show results from two different animals. FIG. 12C shows composite overlay results from the two animals. The X-axis for each graph shows mOX40L+ cells. The Y-axis for each graph shows cyno CD20+ cells.

FIGS. 13A-13C show flow cytometry graphs of spleen cells from non-human primates treated in vivo with intravenous injection of the LNP compositions indicated at the top of the panels (left to right) Compound X/PEG DMG and cholesterol (LNP 1), Compound X/Compound 428 and cholesterol (LNP 2), Compound X/DSPE PEG and cholesterol (LNP 4) or Compound X/beta-sitosterol/cholesterol/PEG DMG (LNP 3), encapsulating an mOX40L-encoding mRNA, showing splenic dendritic cell transfection. FIGS. 13A and 13B show results from two different animals. FIG. 13C shows composite overlay results from the two animals. The X-axis for each graph shows mOX40L+ cells. The Y-axis for each graph shows cyno CD11c+ cells.

FIGS. 14A-14B are graphs summarizing the flow cytometry results for total bone marrow cells from non-human primates treated in vivo with intravenous injection of the LNP compositions indicated at the bottom of the graph (Compound X/PEG DMG and cholesterol (LNP 1), Compound X/Compound 428 and cholesterol (LNP 2), Compound X/DSPE PEG and cholesterol (LNP 4) or Compound X/beta-sitosterol/cholesterol/PEG DMG (LNP 3)) encapsulating an mOX40L-encoding mRNA, showing bone marrow cell transfection. FIG. 14A shows the percentage of mOX40L+ cells in bone marrow from the femur. FIG. 14B shows the percentage of mOX40L+ cells in bone marrow from the humerus.

FIGS. 15A-15C show flow cytometry graphs of spleen cells from non-human primates treated in vivo with intravenous injection of the LNP compositions indicated at the top of the panels (left to right) Compound X/PEG DMG and cholesterol (LNP 1) or Compound X/beta-sitosterol/cholesterol/PEG DMG (LNP 3), encapsulating an mOX40L-encoding mRNA, showing transfection of splenic CD20+ B cells (FIG. 15A), CD3+ T cells (FIG. 15B) and CD11c+ dendritic cells (FIG. 15C).

FIGS. 16A-16B show flow cytometry graphs of bone marrow cells from non-human primates treated in vivo with intravenous injection of the LNP compositions indicated at the top of the panels (left to right) Compound X/PEG DMG and cholesterol (LNP 1) or Compound X/beta-sitosterol/cholesterol/PEG DMG (LNP 3), encapsulating an mOX40L-encoding mRNA, showing transfection of femoral bone marrow cells (FIG. 16A) and humeral bone marrow cells (FIG. 16B).

FIG. 17 shows flow cytometry graphs of non-human primate PBMCs transfected ex vivo with the LNP compositions indicated at the top of the panels (left to right) Compound X/PEG DMG and cholesterol (LNP 1), Compound X/Compound 428 and cholesterol (LNP 2) or Compound X/beta-sitosterol/cholesterol/PEG DMG (LNP 3), encapsulating an mOX40L-encoding mRNA, showing T cell transfection. The X-axis for each graph shows mOX40L+ cells. The Y-axis for each graph shows cyno CD3+ cells.

FIGS. 18A-18D show flow cytometry graphs from human donor bone marrow cells transfected ex vivo with LNP 3 (Compound X/beta-sitosterol/cholesterol/PEG DMG) encapsulating an mOX40L-encoding mRNA, showing transfection of CD45+CD38+CD138+CD19+CD20− plasma cells. FIG. 18A shows the PBS control. FIG. 18B shows results from Donor 1. FIG. 18C shows results from Donor 2. FIG. 18D shows the results from Donor 3.

FIGS. 19A-19C show flow cytometry graphs of splenic cells from rats transfected in vivo with LNP 3 (Compound X/beta-sitosterol/cholesterol/PEG DMG) encapsulating an mOX40L-encoding mRNA at a dose of 0.15 mg/kg, 0.3 mg/kg or 0.6 mg/kg or with PBS control. FIG. 19A shows the results for CD3+ splenic T cells. FIG. 19B shows the results for CD19+ splenic B cells. FIG. 19C shows the results for CD11b+ splenic macrophages.

FIG. 20 shows flow cytometry graphs of splenic cells from rats transfected in vivo with 0.6 mg/kg LNP 3 (Compound X/beta-sitosterol/cholesterol/PEG DMG) encapsulating an mOX40L-encoding mRNA or with PBS control, showing percentage of cells transfected at 24 hours, 96 hours or 168 hours post-dosing.

FIG. 21 shows flow cytometry graphs of splenic cells from rats transfected in vivo with 0.6 mg/kg LNP 3 (Compound X/beta-sitosterol/cholesterol/PEG DMG) encapsulating an mOX40L-encoding mRNA or with PBS control, showing percentage of cells transfected with a treatment regimen of a single dose, one dose every three days for three total doses (Q3Dx3) or one dose a day for three days (QDx3).

FIGS. 22A-22B are bar graphs showing % mOX40L+ cells (left axis) and Median Fluorescence Intensity (MFI) (right axis) for human PBMCs transfected a single time (FIG. 22A) or multiple times (FIG. 22B) with LNP 3 (Compound X/beta-sitosterol/cholesterol/PEG DMG) encapsulating an mOX40L-encoding mRNA. FIG. 22A shows results measured 24, 48 and 72 hours after a single transfection. FIG. 22B shows results measured 24 hours after transfections 1, 2 and 3.

FIGS. 23A-23D show flow cytometry graphs of human T cells transfected ex vivo with PBS, LNP 1 (Compound X/PEG DMG/cholesterol) or LNP 3 (Compound X/beta-sitosterol/cholesterol/PEG DMG) encapsulating an mOX40L-encoding mRNA, showing percentage of cells transfected after LNP interaction times of 15 minutes (FIG. 23A), 60 minutes (FIG. 23B), 4 hours (FIG. 23C) or 24 hours (FIG. 23D).

FIG. 24 is a dot plot showing serum glycoprotein B (gB)-specific IgG titers from mice immunized intramuscularly with an mRNA vaccine encoding cytomegalovirus (CMV) glycoprotein B encapsulated in an LNP containing either Compound Y or Compound Y and beta-sitosterol/cholesterol, as assayed by ELISA.

FIGS. 25A-25B are graphs summarizing flow cytometry results of splenic CD3+ cells (mainly T cells) from C57B16/J mice treated intravenously with either PBS, mOX40L-encoding mRNA encapsulated in an LNP containing Compound X-DSPC-PEG DMG (LNP 1) or mOX40L-encoding mRNA encapsulated in an LNP containing Compound X-beta-sitosterol/cholesterol-DSPC-PEG DMG (LNP 3), showing T cell transfection. FIG. 25A shows the percentage of CD3+ cells expressing mOX40L. FIG. 25B shows the mOX40L mean fluorescence index (MFI) in CD3+ cell population.

FIGS. 26A-26B are graphs summarizing flow cytometry results of splenic CD19+ cells (mainly B cells) from C57B16/J mice treated intravenously with either PBS, mOX40L-encoding mRNA encapsulated in an LNP containing Compound X-DSPC-PEG DMG (LNP 1) or mOX40L-encoding mRNA encapsulated in an LNP containing Compound X-beta-sitosterol/cholesterol-DSPC-PEG DMG (LNP 3), showing B cell transfection. FIG. 26A shows the percentage of CD19+ cells expressing mOX40L. FIG. 26B shows the mOX40L mean fluorescence index (MFI) in CD 19+ cell population.

FIGS. 27A-27B are graphs summarizing flow cytometry results of splenic CD11c+ cells (mainly dendritic cells) from C57B16/J mice treated intravenously with either PBS, mOX40L-encoding mRNA encapsulated in an LNP containing Compound X-DSPC-PEG DMG (LNP 1) or mOX40L-encoding mRNA encapsulated in an LNP containing Compound X-beta-sitosterol/cholesterol-DSPC-PEG DMG (LNP 3), showing dendritic cell transfection. FIG. 27A shows the percentage of CD11c+ cells expressing mOX40L. FIG. 27B shows the mOX40L mean fluorescence index (MFI) in CD11c+ cell population.

FIGS. 28A-28B are graphs summarizing flow cytometry results of total bone marrow cells from C57B16/J mice treated intravenously with either PBS, mOX40L-encoding mRNA encapsulated in an LNP containing Compound X-DSPC-PEG DMG (LNP 1) or mOX40L-encoding mRNA encapsulated in an LNP containing Compound X-beta-sitosterol/cholesterol-DSPC-PEG DMG (LNP 3), showing bone marrow cell transfection. FIG. 28A shows the percentage of bone marrow cells expressing mOX40L. FIG. 28B shows the mOX40L mean fluorescence index (MFI) in bone marrow cell population.

FIG. 29 is a graph showing cytotoxicity of CD33+ AML cells incubated with T cells transfected with an anti-CD33 CAR T mRNA construct encapsulated in LNP 3. T cell:AML cell ratios of 1:1 and 1:5 were tested. A CD34 mRNA construct was used as a control.

FIG. 30 shows results for T cells transfected in vitro with LNPs comprising various different indicated ionizable lipids (amino lipids), showing % of cells with LNP associated and % of cells expressing the protein encoded by the mRNA encapsulated by the LNP.

FIG. 31 shows results for T cells transfected in vitro with LNPs comprising various different indicated sterols, showing % of cells with LNP associated and % of cells expressing the protein encoded by the mRNA encapsulated by the LNP.

FIG. 32 shows results for T cells transfected in vitro with LNPs comprising various different indicated phospholipids, showing % of cells with LNP associated and % of cells expressing the protein encoded by the mRNA encapsulated by the LNP.

FIG. 33 shows results for T cells transfected in vitro with LNPs comprising various different indicated PEG lipid, showing % of cells with LNP associated and % of cells expressing the protein encoded by the mRNA encapsulated by the LNP.

FIG. 34 shows results for T cells transfected in vitro with LNPs comprising various different indicated LNP compositions, showing % of cells with LNP associated and % of cells expressing the protein encoded by the mRNA encapsulated by the LNP.

FIG. 35 shows results for T cells transfected in vitro with LNPs comprising various different indicated sterols, showing % of cells with LNP associated and % of cells expressing the protein encoded by the mRNA encapsulated by the LNP.

FIG. 36 is a bar graph showing flow cytometry results of CD3+ spleen T cells from mice treated in vivo with intravenous injection of different LNP compositions encapsulating an mOX40L-encoding mRNA.

FIG. 37 shows results for monocytes transfected in vitro with LNPs comprising various different indicated ionizable lipids (amino lipids), showing % of cells with LNP associated and % of cells expressing the protein encoded by the mRNA encapsulated by the LNP.

FIG. 38 shows results for monocytes transfected in vitro with LNPs comprising various different indicated sterols, showing % of cells with LNP associated and % of cells expressing the protein encoded by the mRNA encapsulated by the LNP.

FIG. 39 shows results for monocytes transfected in vitro with LNPs comprising various different indicated phospholipids, showing % of cells with LNP associated and % of cells expressing the protein encoded by the mRNA encapsulated by the LNP.

FIG. 40 is a bar graph showing the mean percentage of monocytes expressing the mRNA encapsulated by different LNP compositions upon incubation in vitro.

FIGS. 41A-41B are bar graphs showing human EPO (huEPO) concentration (in mIU/mL), as measured by ELISA, after transfection of T cells with mRNA encoding huEPO encapsulated in either LNP 1 (Compound X/PEG DMG/cholesterol) or LNP 3 (Compound X/beta-sitosterol/cholesterol/PEG DMG). FIG. 41A shows ELISA results for a 1:50 supernatant dilution. FIG. 41B shows ELISA results for a 1:250 supernatant dilution.

FIG. 42 is a bar graph showing the percentage of CD3+ T cells expressing Foxp3 protein after transfection of T cells with mRNA encoding Foxp3 encapsulated in either LNP 1 or LNP 3. Transfection of T cells with mRNA encoding OX40L encapsulated in LNP 3 is also shown as a positive control.

FIG. 43 shows results for T cells transfected in vitro with LNPs comprising various different indicated ionizable lipids (amino lipids), showing % of cells with LNP associated and % of cells expressing the protein encoded by the mRNA encapsulated by the LNP.

FIG. 44 shows results for T cells transfected in vitro with LNPs comprising various different indicated sterols, showing % of cells with LNP associated and % of cells expressing the protein encoded by the mRNA encapsulated by the LNP.

FIG. 45 shows results for T cells transfected in vitro with LNPs comprising various different indicated phospholipids, showing % of cells with LNP associated and % of cells expressing the protein encoded by the mRNA encapsulated by the LNP.

FIG. 46 shows results for T cells transfected in vitro with LNPs comprising various different indicated PEG lipids, showing % of cells with LNP associated and % of cells expressing the protein encoded by the mRNA encapsulated by the LNP.

FIG. 47 shows results for T cells transfected in vitro with LNPs comprising various different indicated lipid formulations, showing % of cells with LNP associated and % of cells expressing the protein encoded by the mRNA encapsulated by the LNP.

### DETAILED DESCRIPTION

The present disclosure provides improved lipid-based compositions, specifically immune cell delivery lipid nanoparticles (LNPs), that comprise immune cell delivery potentiating lipids and which exhibit increased delivery of an agent(s) to immune cells as compared to LNPs lacking immune cell delivery potentiating lipids. In various aspects, the present disclosure provides improved LNPs comprising immune cell delivery potentiating lipids, such LNPs comprising an agent(s) for delivery to an immune cell or population of immune cells, methods for enhancing delivery of an agent (e.g., a nucleic acid molecule) to an immune cell or population of immune cells, methods of delivering such LNPs to subjects that would benefit from modulation of immune cell activity, and methods of treating such subjects. The present disclosure is based, at least in part, on the discovery that certain lipid components of an LNP, when present in the LNP, enhance association of LNPs with immune cells and delivery of an agent into the immune cells, e.g., as demonstrated by expression of nucleic acid molecules by immune cells. Although the LNPs of the disclosure have demonstrated enhanced delivery to immune cells by measuring increased expression of an mRNA in immune cells, the same approach can be demonstrated using knock down of (i.e., decrease of) existing expression, depending on the nucleic acid molecule delivered. In addition, one of ordinary skill in the art will recognize that having demonstrated enhanced delivery to immune cells in this model system using mRNA, other agents may now be delivered to immune cells using the subject immune cell delivery LNPs. Such agents are known in the art and, in one embodiment, an agent comprises or consists of a nucleic acid molecule. In particular, certain potentially therapeutic nucleic acid molecules are known and, in some cases, proteins encoded by such nucleic acid molecules or the nucleic acid molecules themselves are currently being used therapeutically. In view of the advance provided by the subject immune cell enhancing LNPs, improved therapies are possible. Accordingly, in one embodiment, when the agent is a nucleic acid molecule, the enhanced delivery of the nucleic acid molecule can be used to modulate (e.g., increase or decrease) the activation or activity of an immune cell. In some aspects, the agent is a nucleic acid molecule selected from the group consisting of mRNA, RNAi, dsRNA, siRNA, mirs, antagomirs, antisense RNA, ribozyme, CRISPR/Cas9, ssDNA and DNA.

In a particular embodiment, an immune cell delivery LNP enhances delivery of an agent, (e.g., a nucleic acid molecule) to immune cells, such as T cells, B cells, monocytes, and dendritic cells, relative to an LNP lacking an immune cell delivery potentiating lipid. In one embodiment, it has been demonstrated that expression of an mRNA encoding a protein of interest is enhanced in an immune cell when the mRNA is delivered by an immune cell delivery LNP that includes an immune cell delivery potentiating lipid, relative to an LNP lacking the immune cell delivery potentiating lipid. Delivery of an agent associated with (e.g., encapsulated in) immune cell delivery enhancing LNPs to immune cells has been demonstrated in vitro and in vivo, e.g., using expression of proteins encoded by mRNA molecules present in immune cell enhancing LNPs.

As demonstrated herein, immune cell delivery enhancing LNPs have been shown to result in at least about 2-fold increased expression of proteins in immune cells. Delivery to immune cells has also been demonstrated in vivo. Unexpectedly, in vivo delivery of an encapsulated mRNA was demonstrated to at least about 15% of splenic T cells, at least about 25% of splenic B cells, and at least about 40% of dendritic cells following a single intravenous injection of an LNP of the disclosure. Delivery of encapsulated mRNA to greater than 5% of bone marrow cells has also been demonstrated in vitro and in vivo. The levels of delivery demonstrated herein using LNPs comprising immune cell delivery potentiating lipids make in vivo immune therapy possible. The disclosure provides methods for modulation of a variety of immune responses, including upregulation and downregulation of immune responses, in a wide variety of clinical situations, including cancer, infectious diseases, vaccination and autoimmune diseases.

The LNPs of the disclosure are particularly useful to target endogenous T cells, since they provide enhanced delivery to T cells compared to prior art LNPs, thereby avoiding the problems associated with ex vivo expansion of T cells for adoptive transfer. The LNPs can comprise nucleic acid molecules (e.g., mRNA) encoding proteins that traffic immune cells to sites of inflammation, such as tumors. Thus, the present disclosure provides methods to leverage T cells as modified effector or helper cells or as cancer cell targets to thereby modulate immune responses. In another embodiment, T cells can be altered to differentiate into cells that are suppressed and/or that mediate immune suppression using the subject immune cell enhancing LNPs. The enhanced delivery of LNPs into T cells, in particular in vivo, was unexpected as LNPs have not been previously shown to effectively deliver nucleic acid molecules to T cell populations, nor have T cells previously been shown to efficiently take up LNPs.

While not intending to be bound by any particular mechanism or theory, the enhanced delivery of a nucleic acid molecule to immune cells by the LNPs of the disclosure is believed to be due to the presence of an effective amount of an immune cell delivery potentiating lipid, e.g., a cholesterol analog or an amino lipid or combination thereof, that, when present in an LNP, may function by enhancing cellular association and/or uptake, internalization, intracellular trafficking and/or processing, and/or endosomal escape and/or may enhance recognition by and/or binding to immune cells, relative to an LNP lacking the immune cell delivery potentiating lipid. Furthermore, it was observed in in vitro experiments that serum was absolutely required for immune cell uptake/cell association of the LNP. Through depletion of various serum components, it was determined that complement component 1q (C1q) was involved in the uptake of the LNP by the immune cells. Accordingly, while not intending to be bound by any particular mechanism or theory, in one embodiment, an immune cell delivery potentiating lipid of the disclosure binds to C1q or promotes the binding of an LNP comprising such lipid to C1q. Thus, for in vitro use of the LNPs of the disclosure for delivery of a nucleic acid molecule to an immune cell, culture conditions that include C1q are used (e.g., use of culture media that includes serum or addition of exogenous C1q to serum-free media). For in vivo use of the LNPs of the disclosure, the requirement for C1q is supplied by endogenous C1q.

Previous attempts to target LNPs to immune cells have utilized targeting moieties (see e.g., Satsepin, T., et al., International Journal of Nanomedicine 2016, Vol. 11: 3077-3086). The LNPs described herein enhance delivery to immune cells without such targeting moieties. Such targeting moieties can result in unwanted effects, e.g., agonism or antagonism of cell surface receptors, and the LNPs of the disclosure provide improved delivery of encapsulated nucleic acid molecules thereby avoiding the need for such targeting moieties and their undesired effects. Accordingly, in some embodiments, the lipid-based compositions do not include an immune cell targeting moiety, i.e., a moiety that directs the composition to an immune cell. In some embodiments, the lipid-based compositions do not include an antibody with specificity for an immune cell marker. In some embodiments, the lipid-based compositions do not include a ligand that targets the composition to immune cells (e.g., N-acetylgalactosamine or hyaluronan). In one embodiment, the lipid-based composition comprises an mRNA encoding a targeting moiety (e.g., a CAR), but the LNP does not bind to immune cells solely by virtue of said targeting moiety. Rather, in one embodiment, in the case of the instant immune cell delivery enhancing LNPs, association of the LNP with the immune cell and delivery of the agent to the immune cell is enhanced by the presence of immune cell delivery potentiating lipids in the LNP as compared to a control LNP lacking the immune cell delivery potentiating lipids.

The ability to effectively deliver agents (e.g., nucleic acid molecules including mRNA) to immune cells is useful for modulating immune responses by immune cells by, for example, increasing an immune response (e.g., to a cancer antigen or an infectious disease antigen) or decreasing immunosuppression in immune cells (e.g., to restore or enhance effector functions, or modulating immune checkpoint blockade to augment exhausted T cells, such as those expressing PD-1). In another embodiment, nucleic acid molecules that reduce immune cell activation or tolerize immune cells can be delivered to immune cells (e.g., myeloid cells, dendritic cells, T cells and/or B cells), e.g., to reduce autoimmunity. An immune response can be modulated locally or systemically (e.g., by alteration of the activity or function of one or more immune cells). Moreover, cell activity and/or function can be altered in cells to which the LNP is delivered or in cells which interact with and/or are influenced by such cells (e.g., in an autocrine or paracrine fashion).

Immune cell delivery LNPs are useful for delivery of, e.g., nucleic acid molecules which modulate the expression of naturally occurring or engineered molecules. In one embodiment, expression of a soluble/secreted protein is modulated (e.g., a naturally occurring soluble molecule or one that has been modified or engineered to promote improved function/half-life/and/or stability). In another embodiment, expression of an intracellular protein is modulated (e.g., a naturally occurring intracellular protein or an engineered or modified intracellular protein that possesses altered function). In another embodiment, the expression of a transmembrane protein is modulated (e.g., a naturally occurring soluble molecule or one that has been modified or engineered to possess altered function.

In one embodiment the nucleic acid molecule may encode a protein that is not naturally expressed in the immune cell (e.g., a heterologous protein or a modified protein). In one embodiment, the nucleic acid molecule may encode or knock down a protein that is naturally expressed in the immune cell.

For example, in some aspects, LNPs of the disclosure are useful to enhance delivery and expression in immune cells of an mRNA encoding a soluble/secreted protein, a transmembrane protein, or an intracellular protein. Exemplary transmembrane proteins may impart a new binding specificity to an immune cell. Exemplary intracellular molecules may modulate cell signaling or cell fate. For example, exemplary proteins that may be expressed include: a cytokine, a chemokine, a costimulatory factor, a T cell Receptors (TcRs), a chimeric antigen receptor (CAR), a recruitment factor, a transcription factor, an effector molecule, an MHC molecule, an enzyme or combination thereof.

In other aspects, LNPs of the disclosure are useful to genetically engineer immune effector cells to express mRNA encoding chimeric antigen receptors (CARs) that redirect cytotoxicity toward tumor cells. CARs, which are modified transmembrane proteins, comprise a ligand- or antigen-specific recognition domain that bind to a specific target ligand or antigen. In some aspects, the LNPs of the disclosure are useful to deliver to T cells an mRNA encoding a CAR that binds a cancer antigen to increase an immune response to the cancer antigen. For example, a T cell response to CD33+ acute myelocytic leukemia (AML) cells can be induced or increased by administration of an LNP of the disclosure comprising an mRNA encoding an anti-CD33 CAR.

The disclosure also provides methods for use of multiple LNPs in combination for delivery of the same (e.g., in different LNPs) or different agents, e.g., nucleic acid molecules (e.g., in the same LNP or different LNPs (e.g., one that is an immune cell delivery enhancing LNP and one that is not) to deliver nucleic acid molecules to immune cells or to different cell populations.

### Immune Cell Delivery LNPs

Immune cell delivery LNPs can be characterized in that they result in increased delivery of agents to immune cells as compared to a control LNP (e.g., an LNP lacking the immune cell delivery potentiating lipid). In particular, in one embodiment, immune cell delivery LNPs result in an increase (e.g., a 2-fold or more increase) in the percentage of LNPs associated with immune cells as compared to a control LNP or an increase (e.g., a 2-fold or more increase) in the percentage of immune cells expressing the agent carried by the LNP (e.g., expressing the protein encoded by the mRNA associated with/encapsulated by the LNP) as compared to a control LNP. In another embodiment, immune cell delivery LNPs result in increased binding to C1q and/or increased uptake of C1q-bound LNP into the immune cells (e.g., via opsonization) as compared to a control LNP (e.g., an LNP lacking the immune cell delivery potentiating lipid).

In another embodiment, immune cell delivery LNPs result in an increase in the delivery of an agent (e.g., a nucleic acid molecule) to immune cells as compared to a control LNP. In one embodiment, immune cell delivery LNPs result in an increase in the delivery of a nucleic acid molecule agent to T cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to myeloid cells as compared to a control LNP.

In one embodiment, when the nucleic acid molecule is an mRNA, an increase in the delivery of a nucleic acid agent to immune cells can be measured by the ability of an LNP to effect at least about 2-fold greater expression of a protein molecule encoded by the mRNA in immune cells, (e.g., T cells) as compared to a control LNP.

Immune cell delivery LNPs comprise an (i) ionizable lipid; (ii) sterol or other structural lipid; (iii) a non-cationic helper lipid or phospholipid; a (iv) PEG lipid and (v) an agent (e.g., a nucleic acid molecule) encapsulated in and/or associated with the LNP, wherein one or more of (i) the ionizable lipid or (ii) the structural lipid or sterol in an immune cell delivery LNPs comprises an effective amount of an immune cell delivery potentiating lipid.

In another embodiment, an immune cell delivery lipid nanoparticle of the disclosure comprises:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell. In one embodiment, enhanced delivery is relative to a lipid nanoparticle lacking the immune cell delivery potentiating lipid. In another embodiment, the enhanced delivery is relative to a suitable control.

In another embodiment, an immune cell delivery lipid nanoparticle of the disclosure comprises:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q or promotes (e.g., increases, stimulates, enhances) the binding of the LNP to C1q, as compared to a control LNP lacking the C1q binding lipid.

In another embodiment, an immune cell delivery lipid nanoparticle of the disclosure comprises:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid

wherein one or more of (i) the ionizable lipid or (ii) the sterol or other structural lipid binds to C1q or promotes (e.g., increases, stimulates, enhances) the binding of the LNP to C1q, as compared to a control LNP (e.g., an LNP lacking (i) the ionizable lipid or (ii) the sterol or other structural lipid).

In another aspect, the disclosure provides a method of screening for an immune cell delivery lipid, the method comprising contacting a test LNP comprising a test immune cell delivery lipid with C1q, and measuring binding to C1q, wherein a test immune cell delivery lipid is selected as an immune cell delivery lipid when it binds to C1q or promotes (e.g., increases, stimulates, enhances) the binding of the LNP comprising it to C1q.

### Lipid Content of LNPs

As set forth above, with respect to lipids, immune cell delivery LNPs comprise an (i) ionizable lipid; (ii) sterol or other structural lipid; (iii) a non-cationic helper lipid or phospholipid; a (iv) PEG lipid, wherein one or more of (i) the ionizable lipid or (ii) the structural lipid or sterol in an immune cell delivery LNPs comprises an effective amount of an immune cell delivery potentiating lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

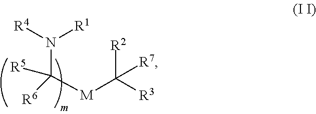

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III):

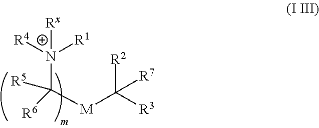

### or its N-oxide,

or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥOH, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl; each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I) includes those of Formula (IA):

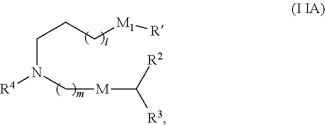

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

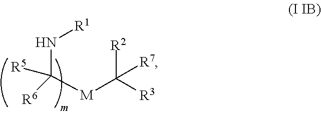

or its N-oxide, or a salt or isomer thereof in which all variables are as defined herein. For example, m is selected from 5, 6, 7, 8, and 9; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (II):

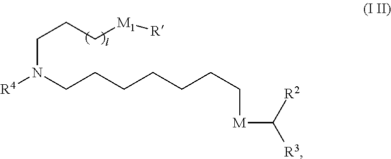

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (I VI):

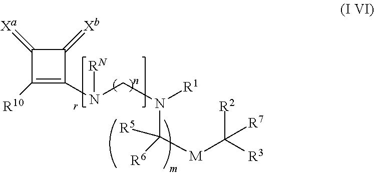

### or its N-oxide,

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

each R is independently selected from the group consisting of H, C₁₋₃ alkyl, and C₂₋₃ alkenyl;

R^{N} is H, or C₁₋₃ alkyl;

each R′ is independently selected from the group consisting of C₁₋₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I;

X^{a} and X^{b} are each independently O or S;
R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;
m is selected from 5, 6, 7, 8, 9, 10, 11, 12, and 13;
n is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, 4, and 5.

In one embodiment, a subset of compounds of Formula (VI) includes those of Formula (VI-a):

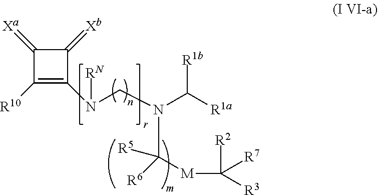

### or its N-oxide,

or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

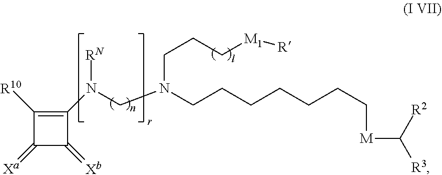

or its N-oxide, or a salt or isomer thereof, wherein

1 is selected from 1, 2, 3, 4, and 5;

M₁ is a bond or M′; and

R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

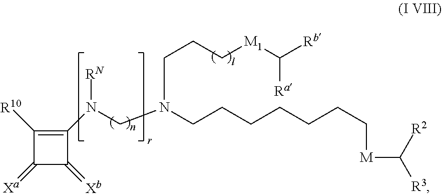

or its N-oxide, or a salt or isomer thereof, wherein

l is selected from 1, 2, 3, 4, and 5;

M₁ is a bond or M′; and

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b}— is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa):

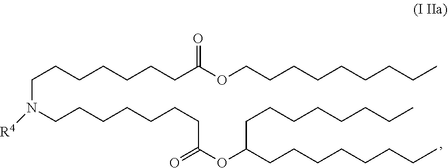

or their N-oxides, or salts or isomers thereof, wherein R⁴ is as described herein.

In another embodiment, the compounds of Formula (I) are of Formula (IIb):

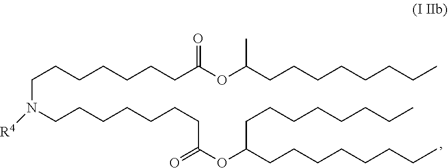

or their N-oxides, or salts or isomers thereof, wherein R⁴ is as described herein.

In another embodiment, the compounds of Formula (I) are of Formula (IIc) or (IIe):

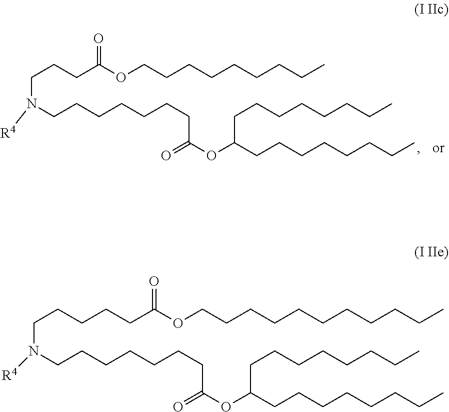

or their N-oxides, or salts or isomers thereof, wherein R⁴ is as described herein.

In another embodiment, the compounds of Formula (I I) are of Formula (I IIf):

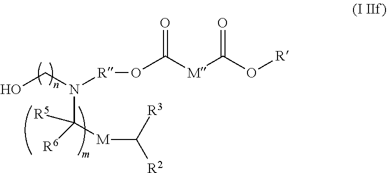

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

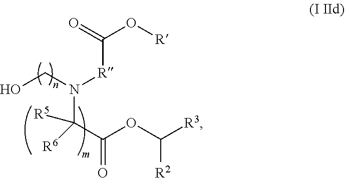

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

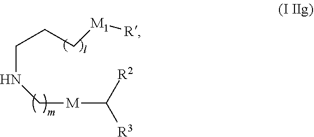

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

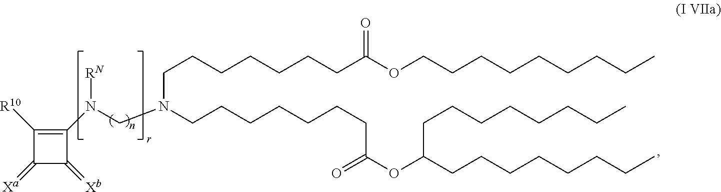

or its N-oxide, or a salt or isomer thereof.

In another embodiment, a subset of compounds of Formula (I VI) includes those of Formula (I VIIIa):

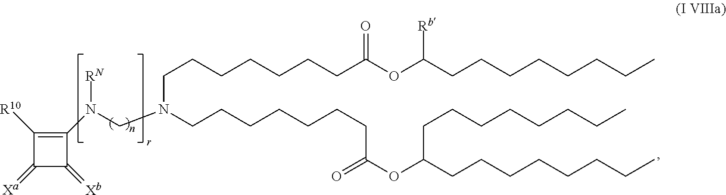

or its N-oxide, or a salt or isomer thereof.

In another embodiment, a subset of compounds of Formula (I VI) includes those of Formula (I VIIIb):

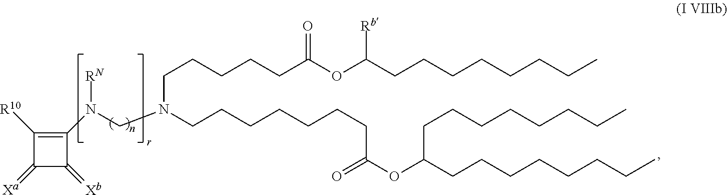

or its N-oxide, or a salt or isomer thereof.

In another embodiment, a subset of compounds of Formula (I VI) includes those of Formula (I VIIb-1):

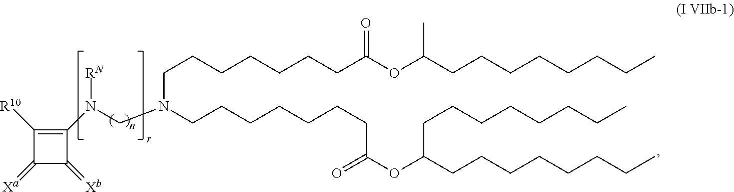

or its N-oxide, or a salt or isomer thereof.

In another embodiment, a subset of compounds of Formula (I VI) includes those of Formula (I VIIb-2):

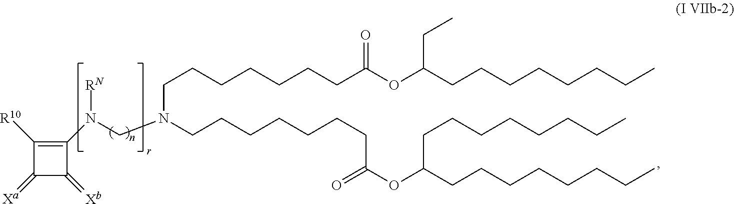

or its N-oxide, or a salt or isomer thereof.

In another embodiment, a subset of compounds of Formula (I VI) includes those of Formula (I VIIb-3):

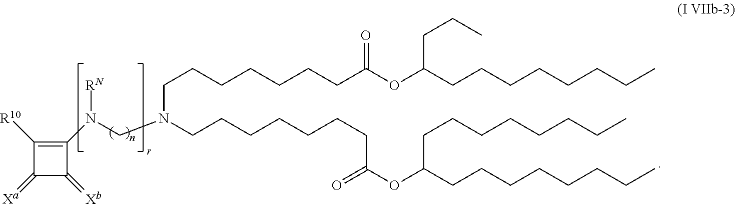

or its N-oxide, or a salt or isomer thereof. In another embodiment, a subset of compounds of Formula (VI) includes those of Formula (VIIc):

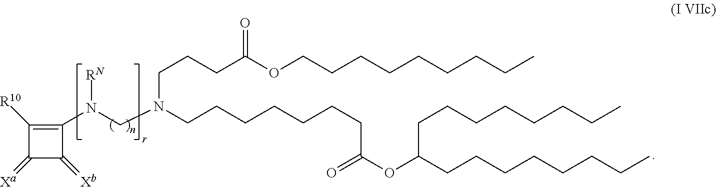

In another embodiment, a subset of compounds of Formula (I VI) includes those of Formula (VIId):

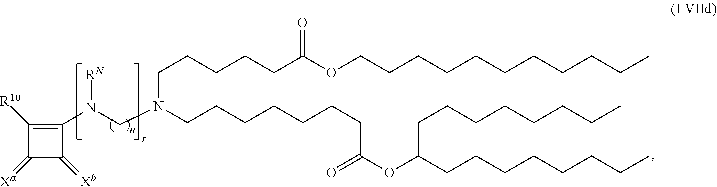

or its N-oxide, or a salt or isomer thereof.

In another embodiment, a subset of compounds of Formula (I VI) includes those of Formula (I VIIIc):

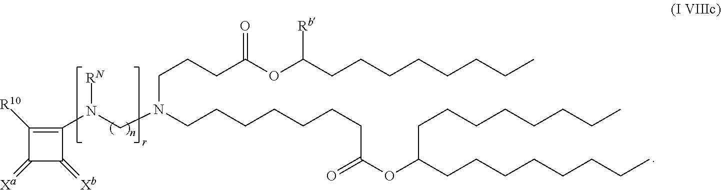

In another embodiment, a subset of compounds of Formula I VI) includes those of Formula (I VIIId):

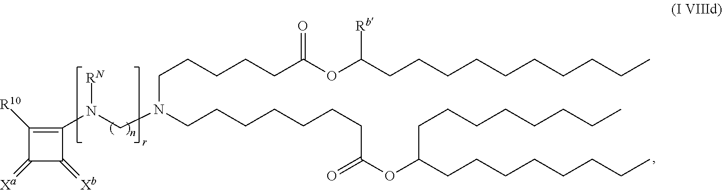

or its N-oxide, or a salt or isomer thereof.

The compounds of any one of formulae (I I), (I IA), (I IB), (I II), (I IIa), (I IIb), (I IIc), (I IId), (I IIe), (I IIf), (I IIg), I (III), (I VI), (I VI-a), (I VII), (I VIII), (I VIIa), (I VIIa), (I VIIIb), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment, R⁴ is is —(CH₂)ₒC(R¹⁰)₂(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is C₁₋₃ alkyl or C₂₋₃ alkenyl. In another embodiment, each R¹⁰ is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is OH.

In another embodiment, R⁴ is unsubstituted C₁₋₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆ dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

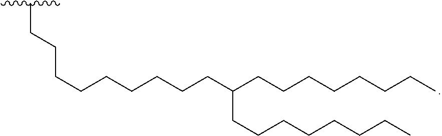 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

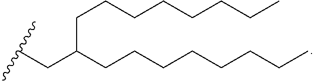 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

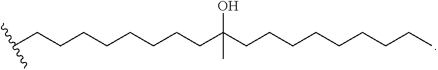 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

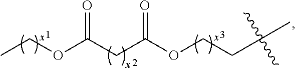

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is

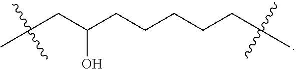 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl, each of which is either linear or branched.

In some embodiments, R′ is linear. In some embodiments, R′ is branched.

In some embodiments, R′ is

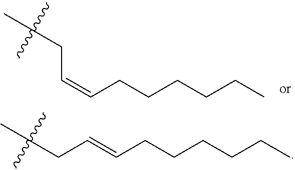

In some embodiments, R′ is

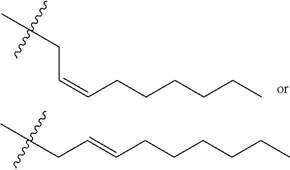

or and M′ is —OC(O)—. In other embodiments, R′ is

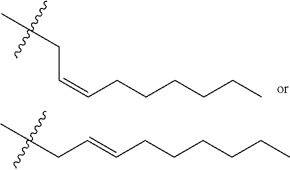

### or and M′ is —C(O)O—.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

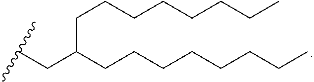 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl, or C(O)O-alkyl or OC(O)-alkyl such as C(O)OCH₃ or OC(O)CH₃). For example, R′ is

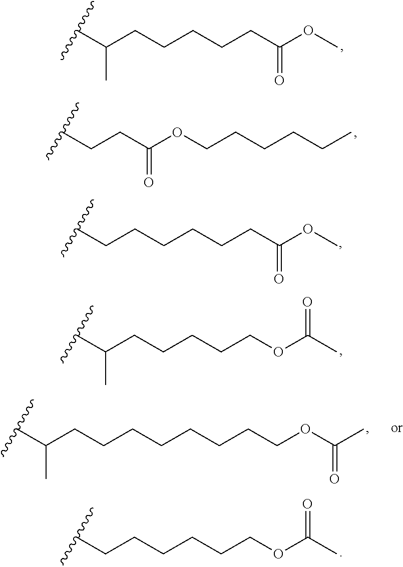

In certain embodiments, R′ is branched C₁₋₁₈ alkyl. For example, R′ is

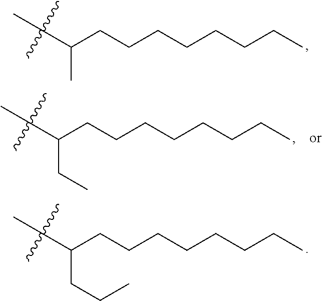

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, C₁₄ alkyl, or C₁₅ alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

In certain embodiments, Q is thiourea or an isostere thereof, e.g.,

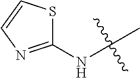 [SMILES: *Nc1nccs1]

or —NHC(═NR⁹)N(R)₂.

In certain embodiments, Q is —C(═NR⁹)N(R)₂. For example, when Q is —C(═NR⁹)N(R)₂, n is 4 or 5. For example, R⁹ is —S(O)₂N(R)₂.

In certain embodiments, Q is —C(═NR⁹)R or —C(O)N(R)OR, e.g., —CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyl)amino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O(CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

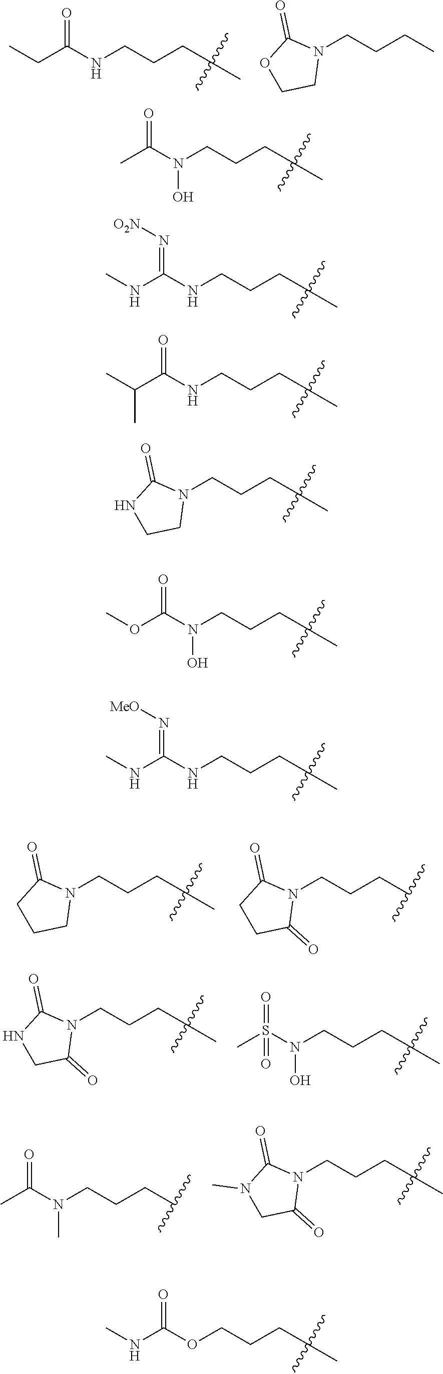

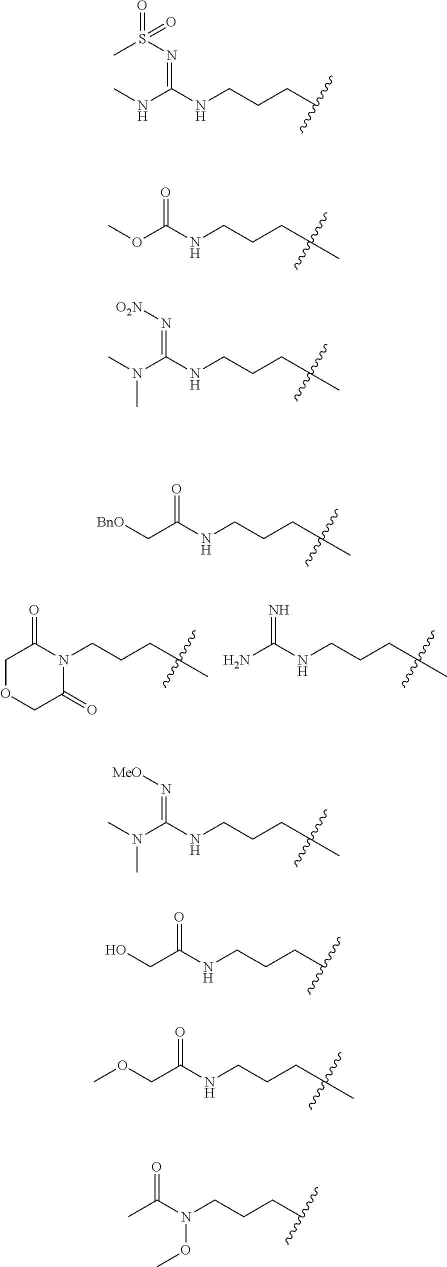

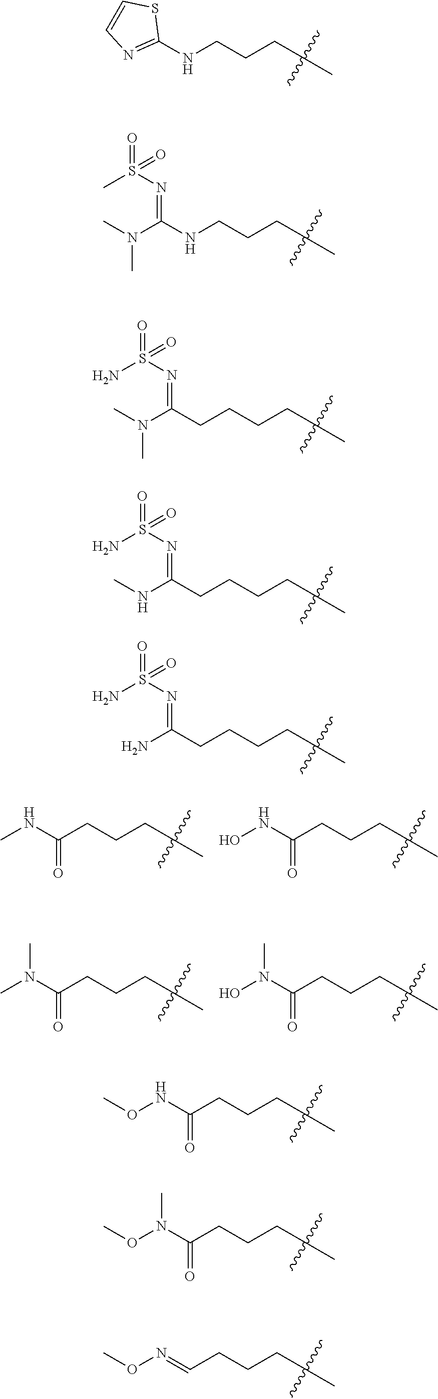

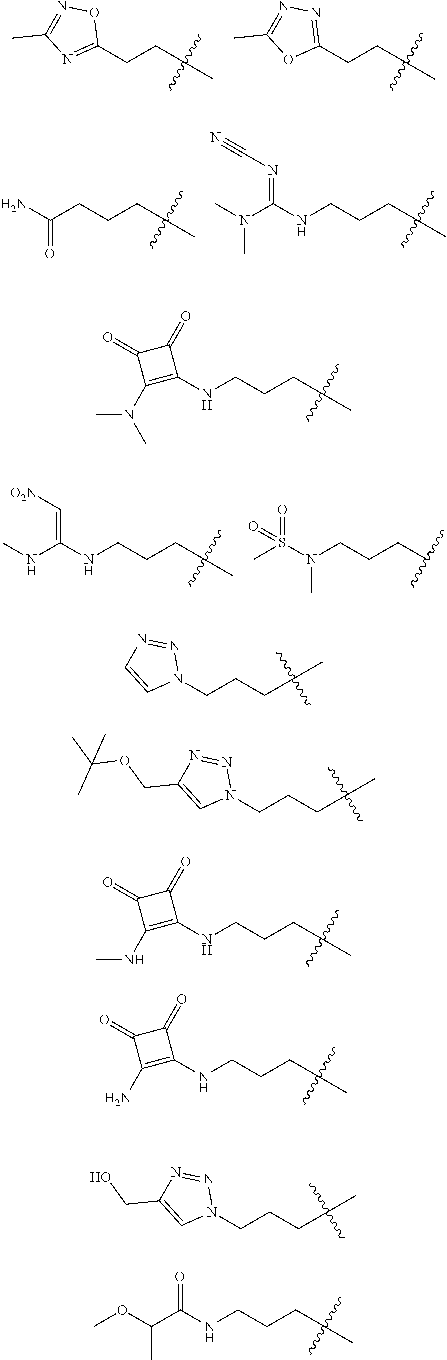

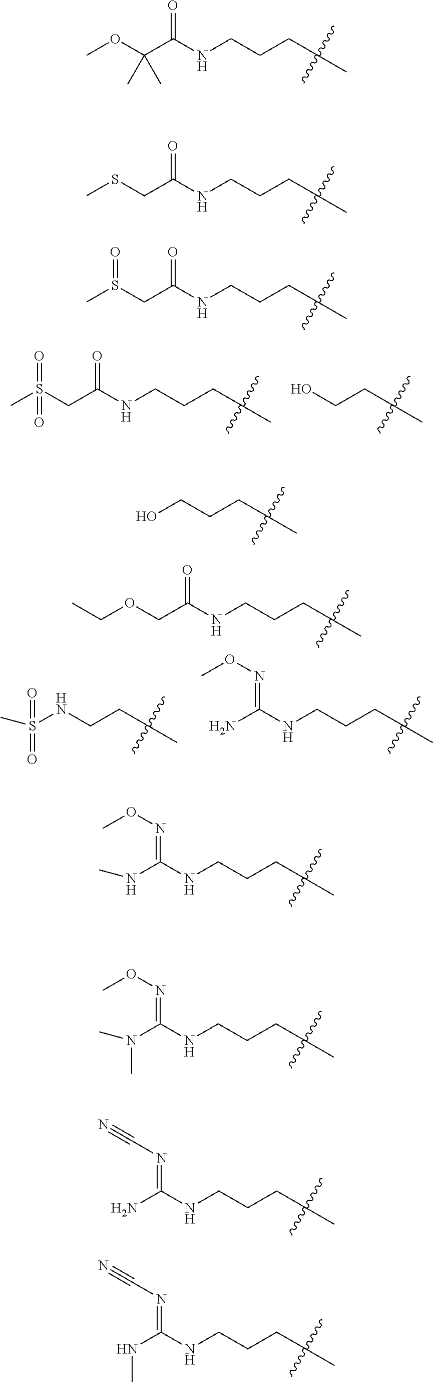

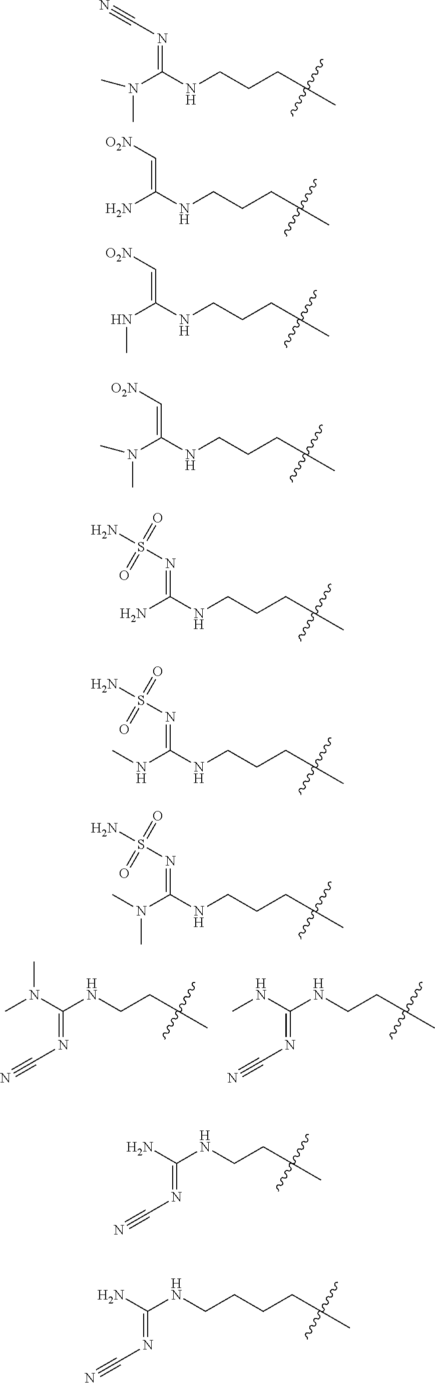

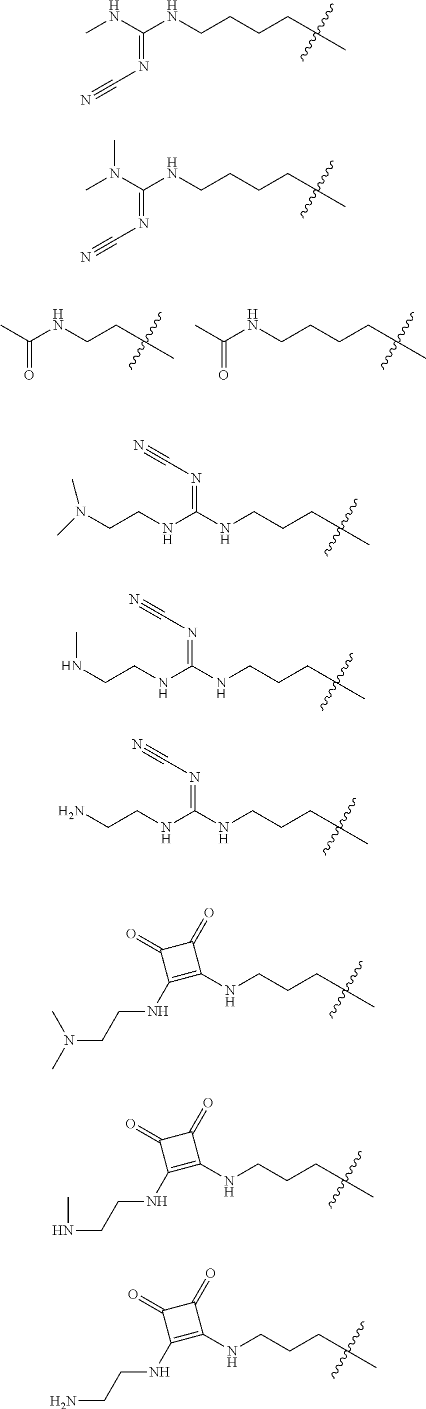

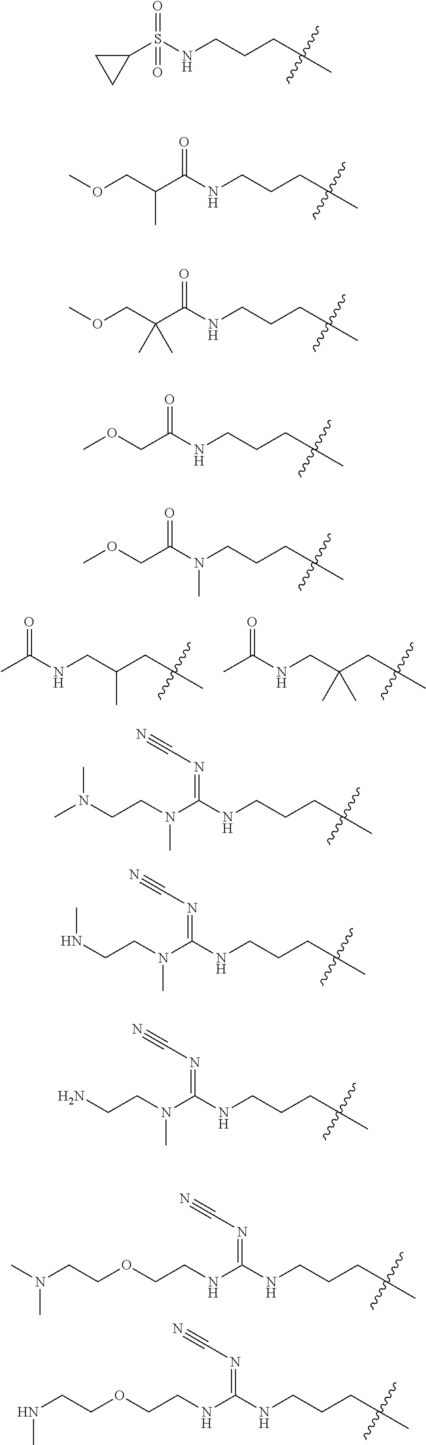

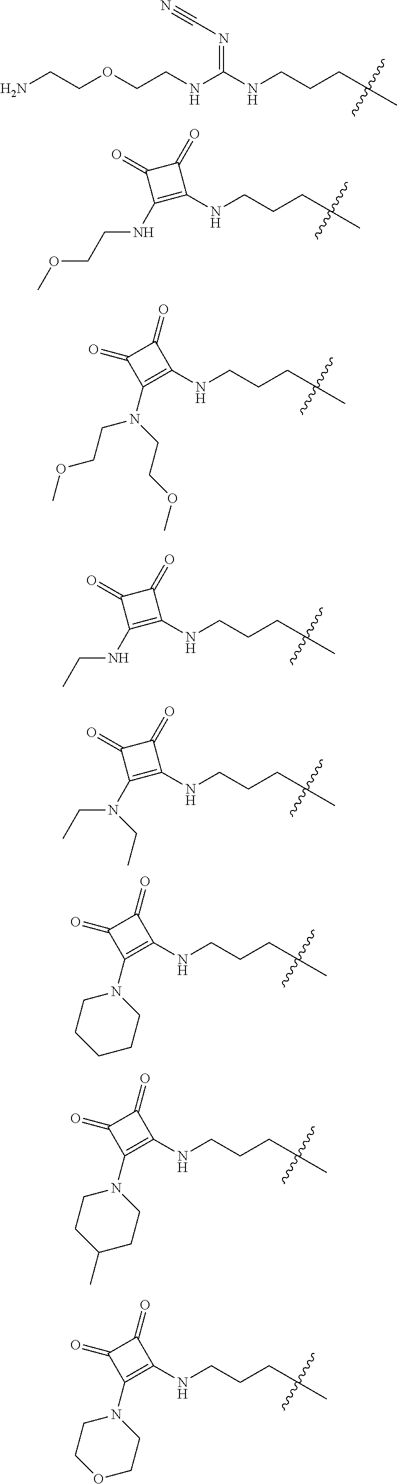

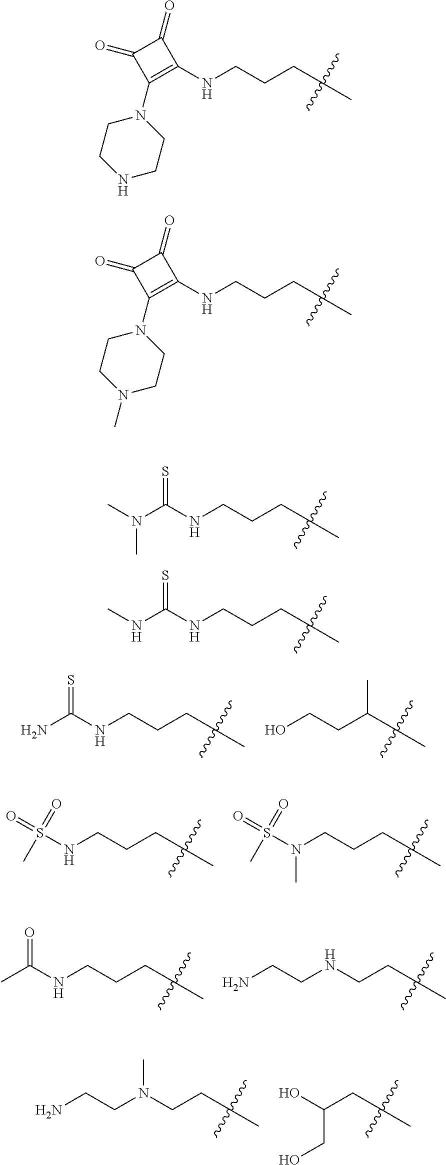

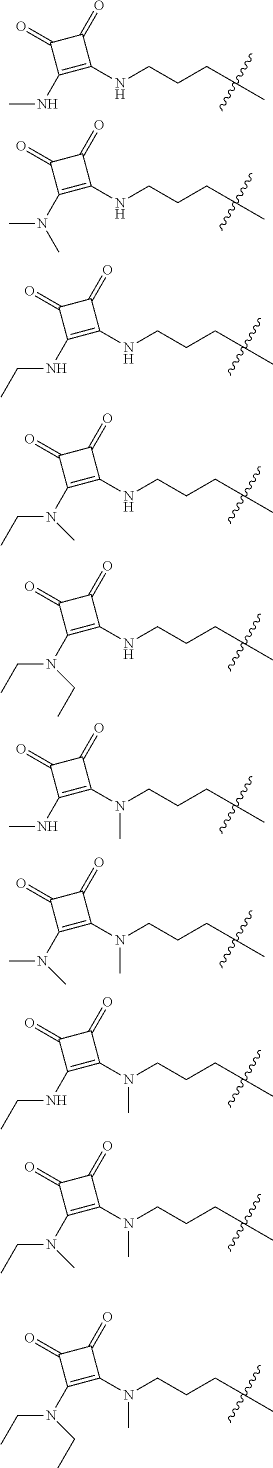

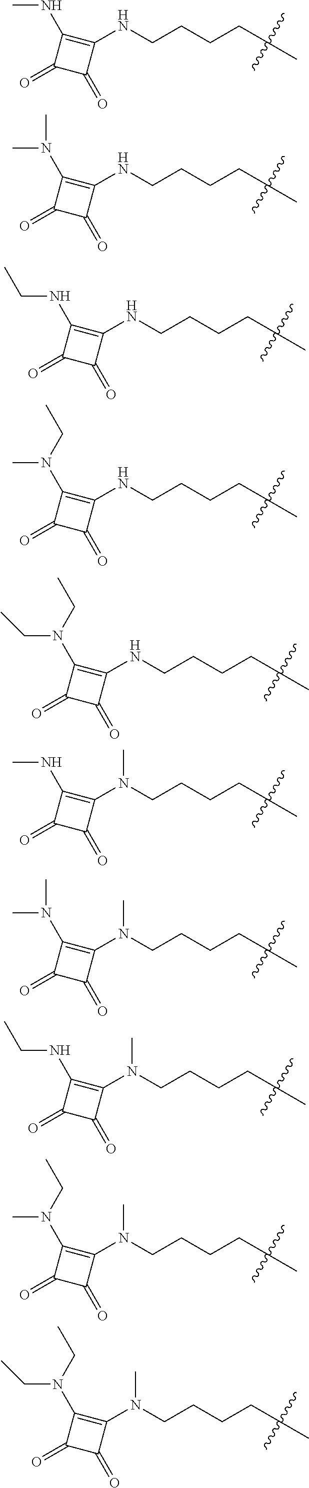

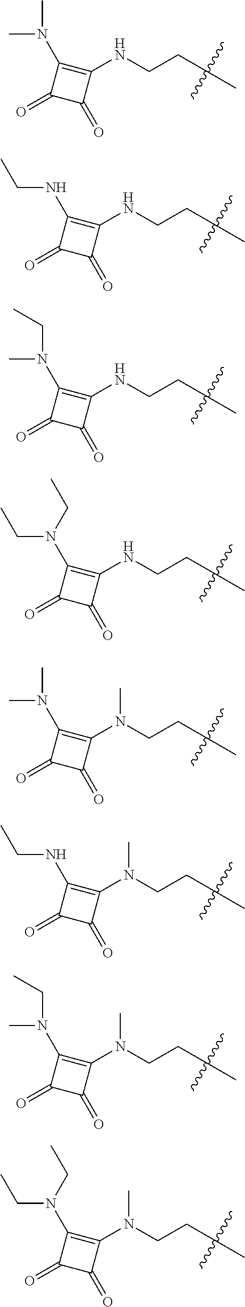

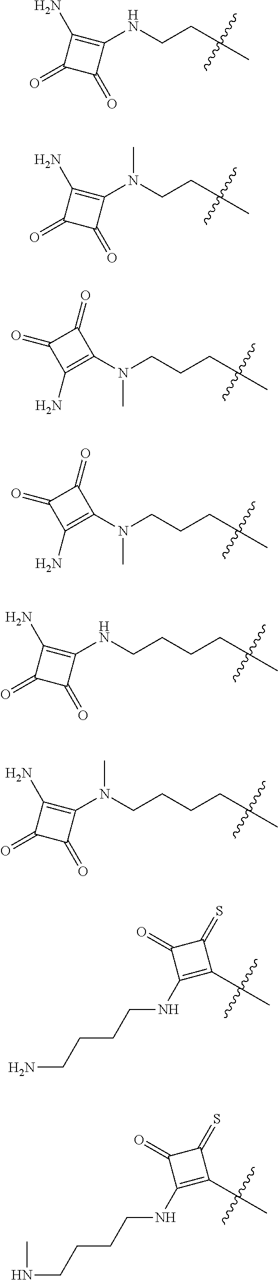

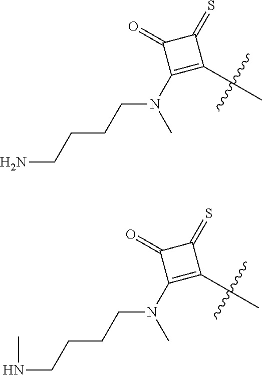 [SMILES: *c1c(N(C)CCCCN)c(=O)c1=S ; *c1c(N(C)CCCCNC)c(=O)c1=S]

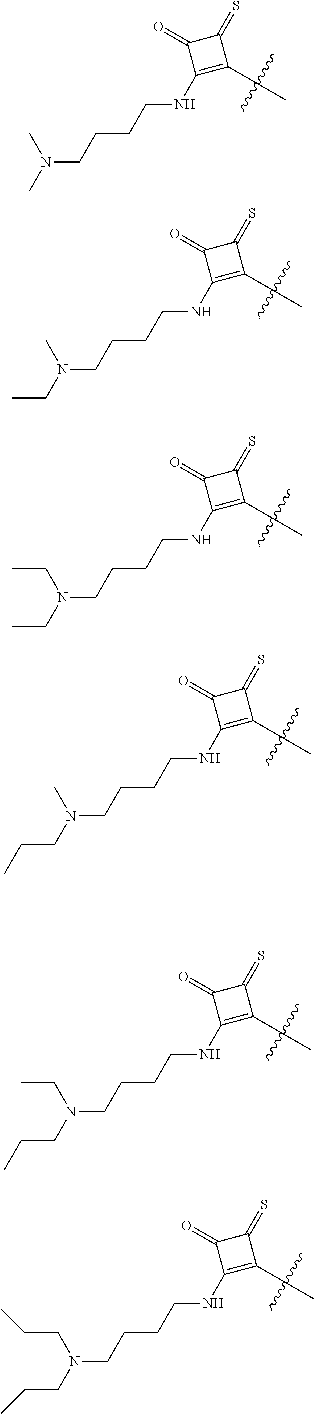 [SMILES: *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

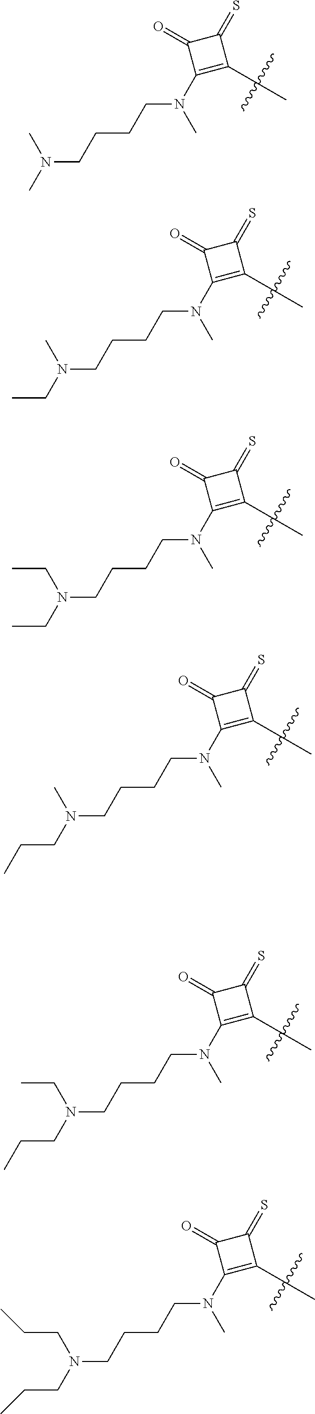 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

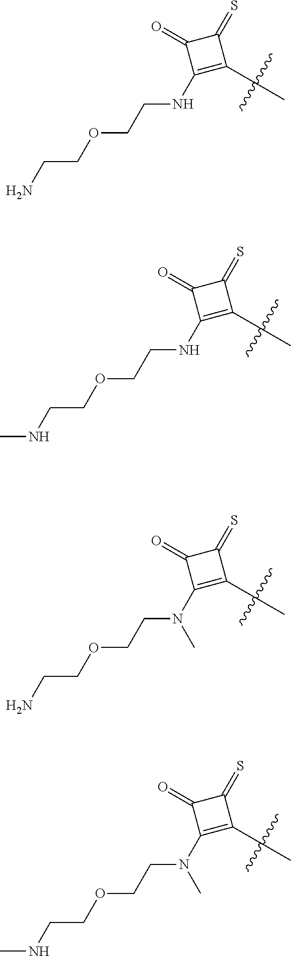 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

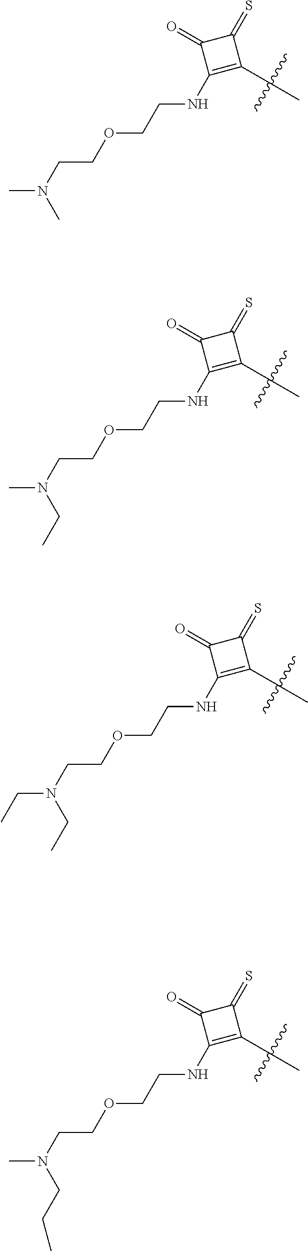 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S]

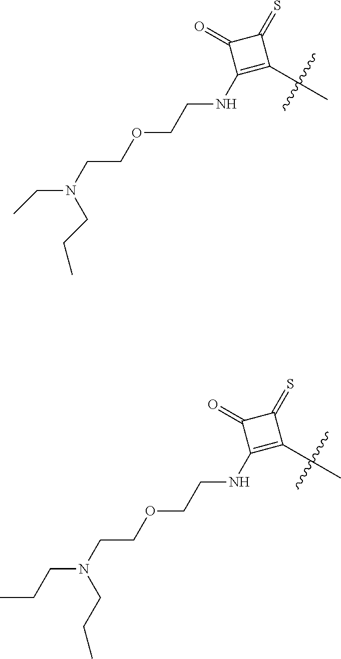 [SMILES: *c1c(NCCOCCN(CC)CCC)c(=O)c1=S ; *c1c(NCCOCCN(CCC)CCC)c(=O)c1=S]

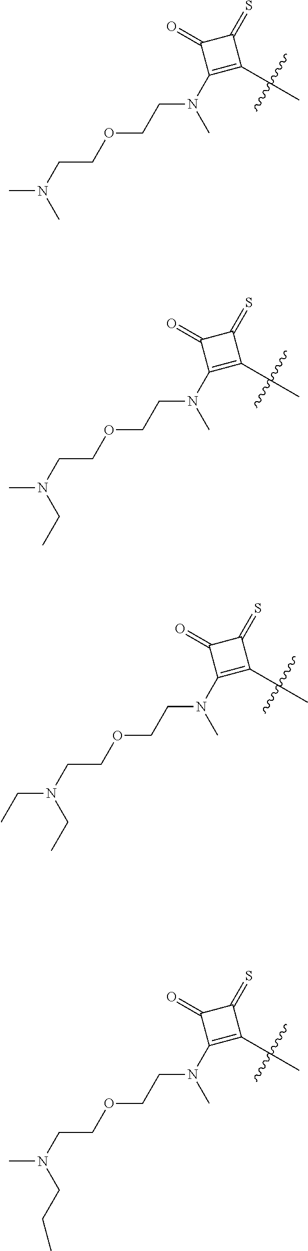 [SMILES: *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

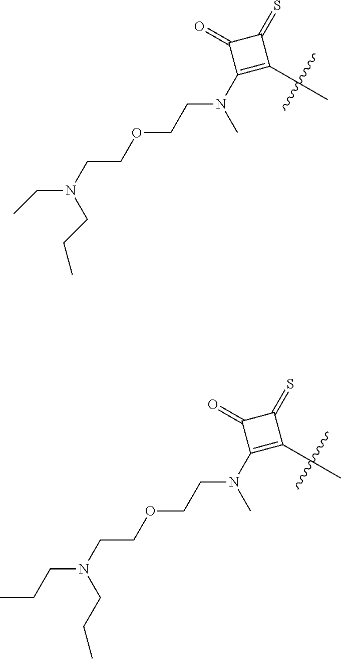 [SMILES: *c1c(N(C)CCOCCN(CC)CCC)c(=O)c1=S ; *c1c(N(C)CCOCCN(CCC)CCC)c(=O)c1=S]

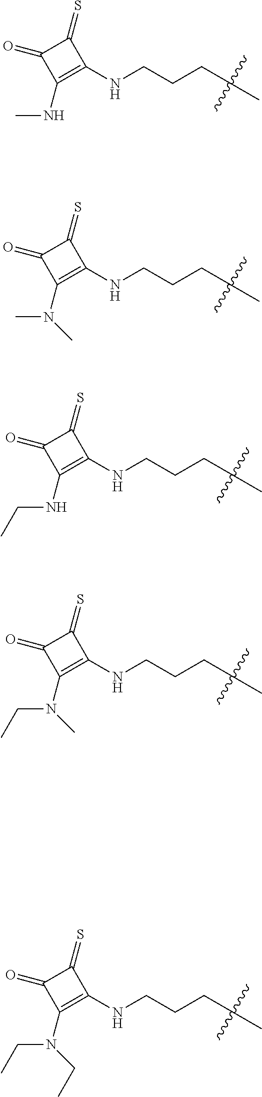 [SMILES: *CCCNc1c(NC)c(=O)c1=S ; *CCCNc1c(N(C)C)c(=O)c1=S ; *CCCNc1c(NCC)c(=O)c1=S ; *CCCNc1c(N(C)CC)c(=O)c1=S ; *CCCNc1c(N(CC)CC)c(=O)c1=S]

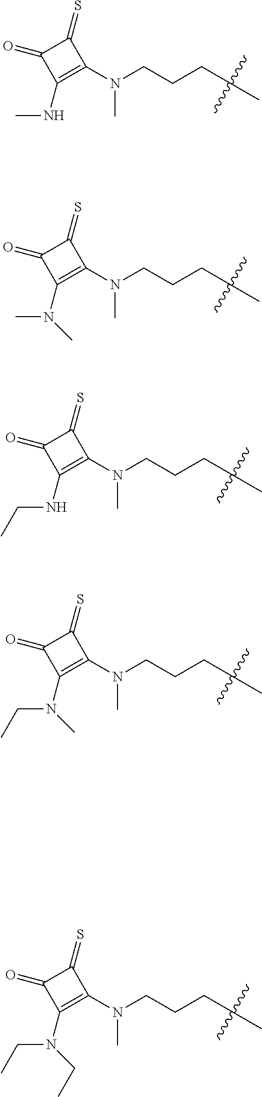 [SMILES: *CCCN(C)c1c(NC)c(=O)c1=S ; *CCCN(C)c1c(N(C)C)c(=O)c1=S ; *CCCN(C)c1c(NCC)c(=O)c1=S ; *CCCN(C)c1c(N(C)CC)c(=O)c1=S ; *CCCN(C)c1c(N(CC)CC)c(=O)c1=S]

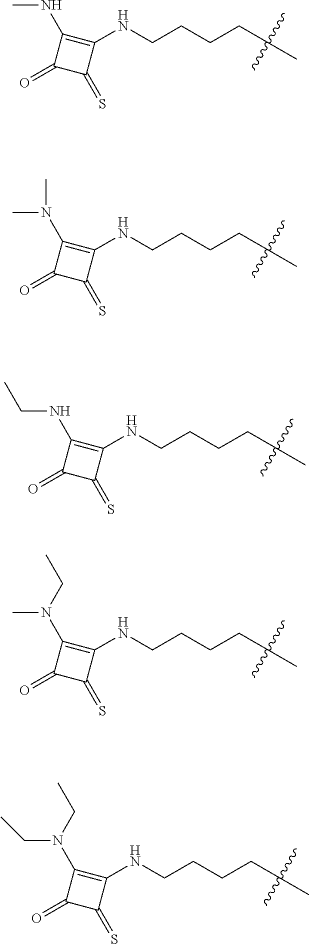 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

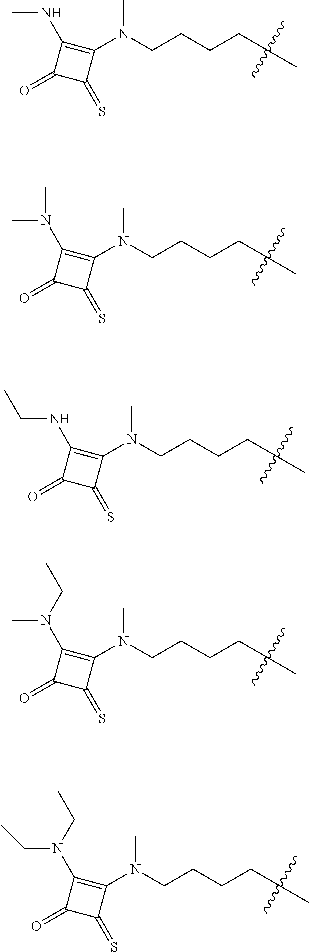 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

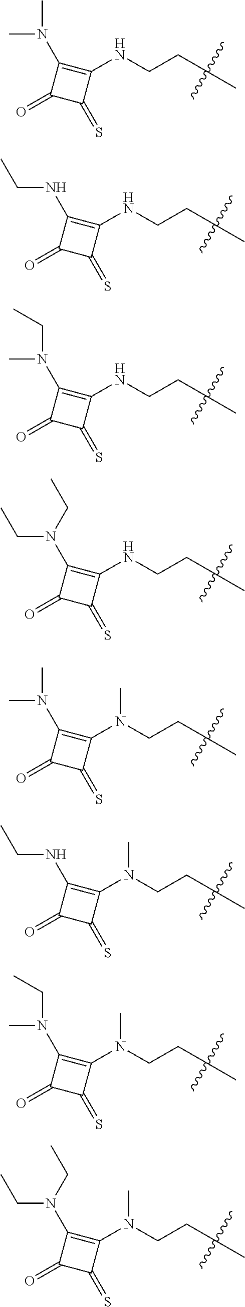

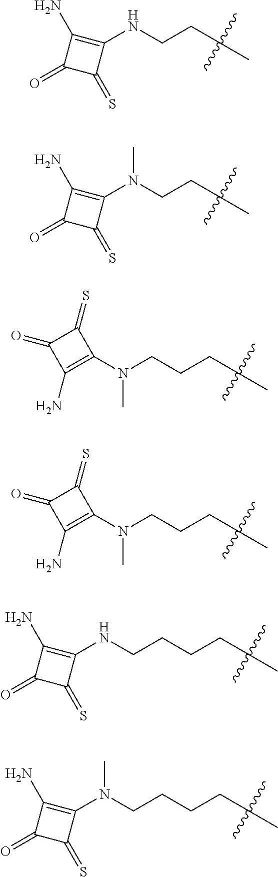

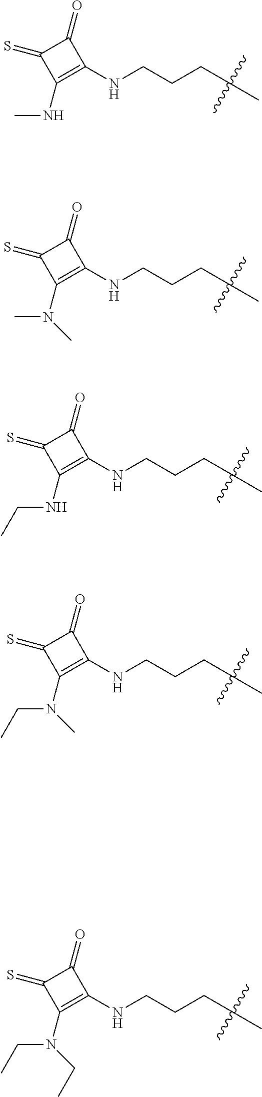 [SMILES: *CCCNc1c(NC)c(=S)c1=O ; *CCCNc1c(N(C)C)c(=S)c1=O ; *CCCNc1c(NCC)c(=S)c1=O ; *CCCNc1c(N(C)CC)c(=S)c1=O ; *CCCNc1c(N(CC)CC)c(=S)c1=O]

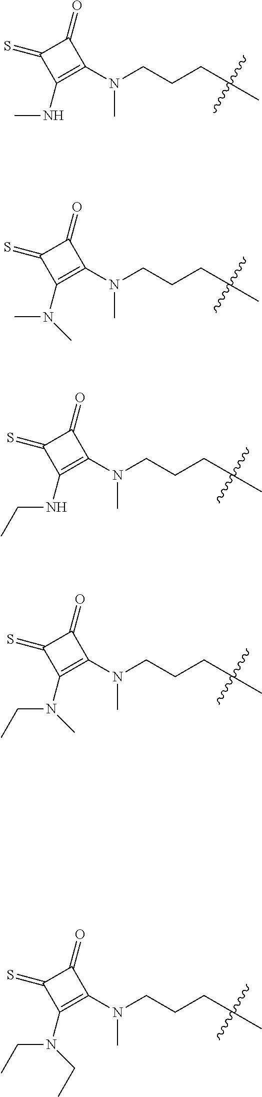 [SMILES: *CCCN(C)c1c(NC)c(=S)c1=O ; *CCCN(C)c1c(N(C)C)c(=S)c1=O ; *CCCN(C)c1c(NCC)c(=S)c1=O ; *CCCN(C)c1c(N(C)CC)c(=S)c1=O ; *CCCN(C)c1c(N(CC)CC)c(=S)c1=O]

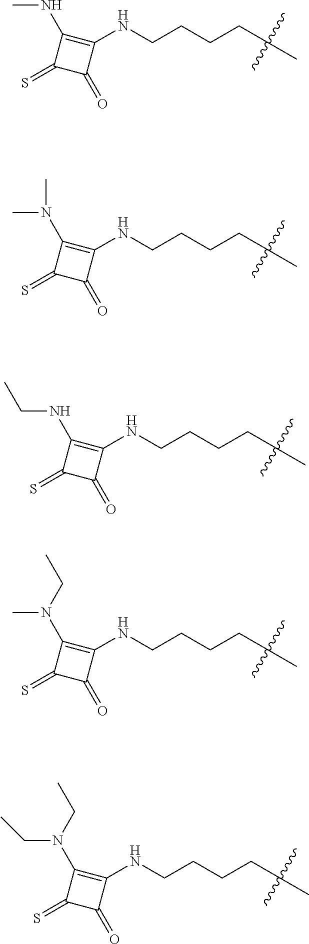 [SMILES: *CCCCNc1c(NC)c(=S)c1=O ; *CCCCNc1c(N(C)C)c(=S)c1=O ; *CCCCNc1c(NCC)c(=S)c1=O ; *CCCCNc1c(N(C)CC)c(=S)c1=O ; *CCCCNc1c(N(CC)CC)c(=S)c1=O]

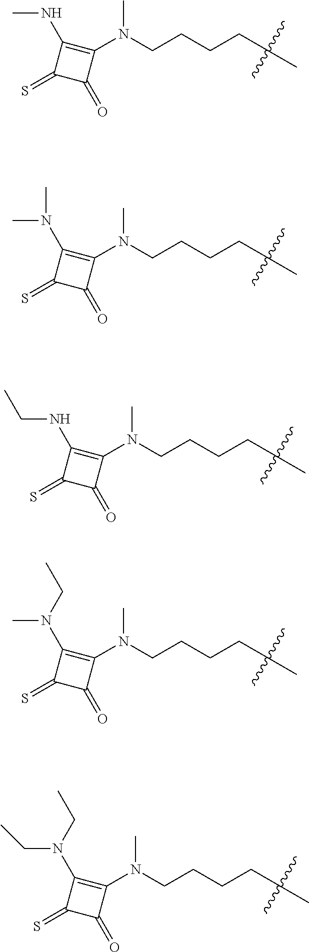 [SMILES: *CCCCN(C)c1c(NC)c(=S)c1=O ; *CCCCN(C)c1c(N(C)C)c(=S)c1=O ; *CCCCN(C)c1c(NCC)c(=S)c1=O ; *CCCCN(C)c1c(N(C)CC)c(=S)c1=O ; *CCCCN(C)c1c(N(CC)CC)c(=S)c1=O]

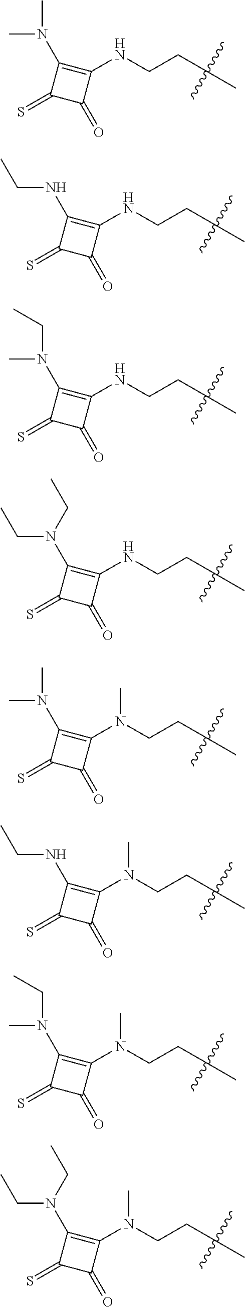

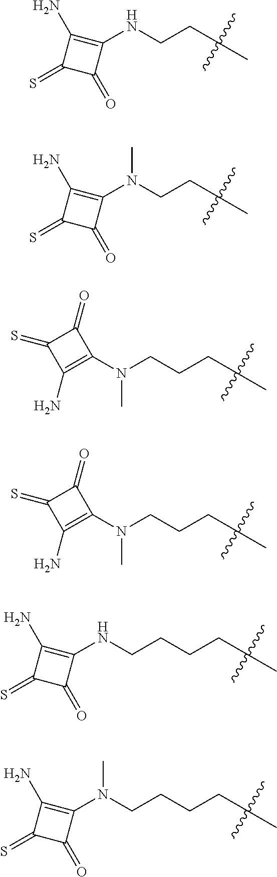

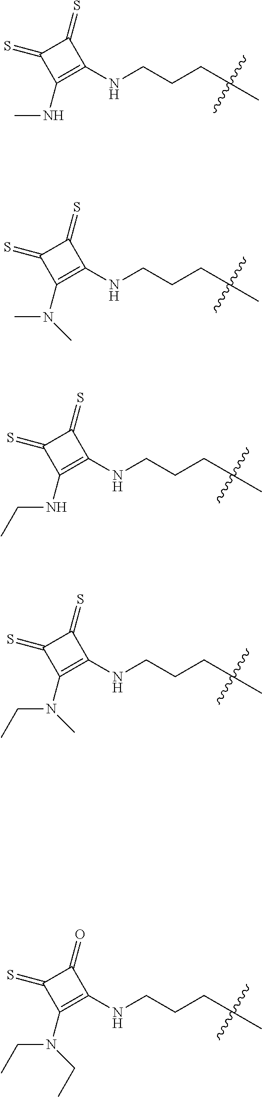 [SMILES: *CCCNc1c(NC)c(=S)c1=S ; *CCCNc1c(N(C)C)c(=S)c1=S ; *CCCNc1c(NCC)c(=S)c1=S ; *CCCNc1c(N(C)CC)c(=S)c1=S ; *CCCNc1c(N(CC)CC)c(=S)c1=O]

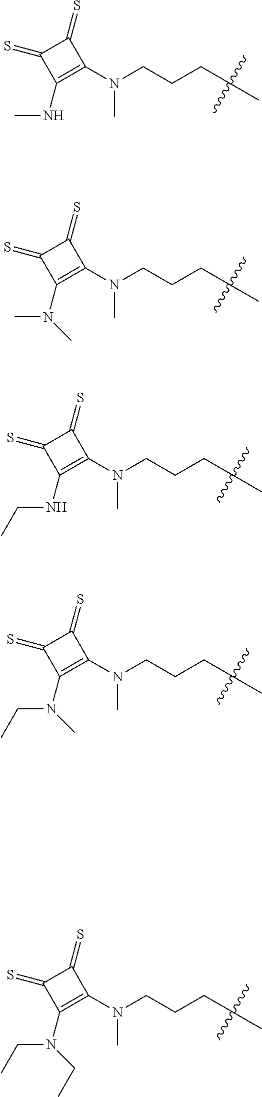 [SMILES: *CCCN(C)c1c(NC)c(=S)c1=S ; *CCCN(C)c1c(N(C)C)c(=S)c1=S ; *CCCN(C)c1c(NCC)c(=S)c1=S ; *CCCN(C)c1c(N(C)CC)c(=S)c1=S ; *CCCN(C)c1c(N(CC)CC)c(=S)c1=S]

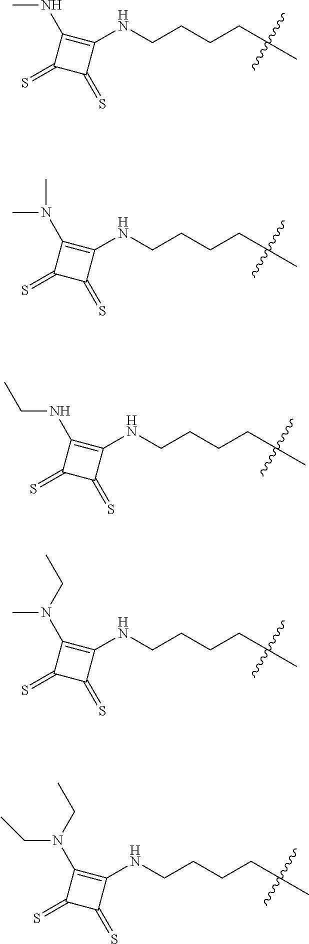 [SMILES: *CCCCNc1c(NC)c(=S)c1=S ; *CCCCNc1c(N(C)C)c(=S)c1=S ; *CCCCNc1c(NCC)c(=S)c1=S ; *CCCCNc1c(N(C)CC)c(=S)c1=S ; *CCCCNc1c(N(CC)CC)c(=S)c1=S]

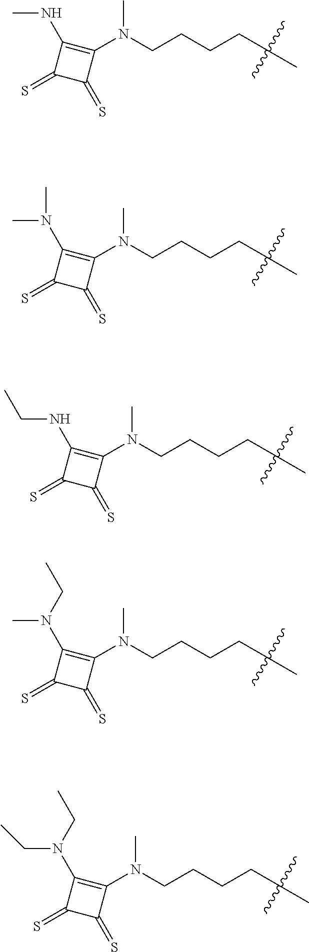 [SMILES: *CCCCN(C)c1c(NC)c(=S)c1=S ; *CCCCN(C)c1c(N(C)C)c(=S)c1=S ; *CCCCN(C)c1c(NCC)c(=S)c1=S ; *CCCCN(C)c1c(N(C)CC)c(=S)c1=S ; *CCCCN(C)c1c(N(CC)CC)c(=S)c1=S]

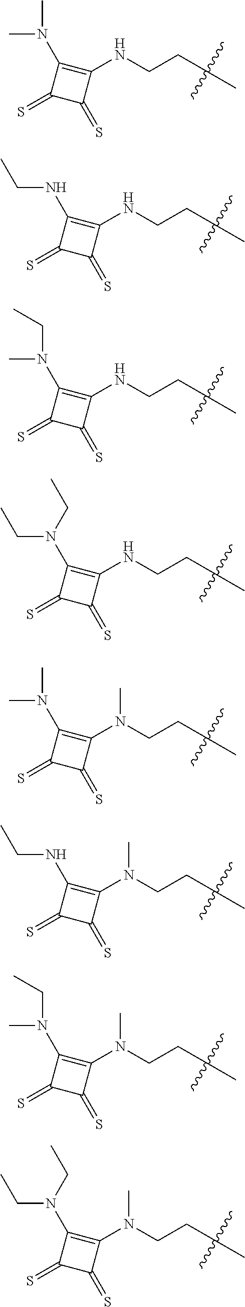

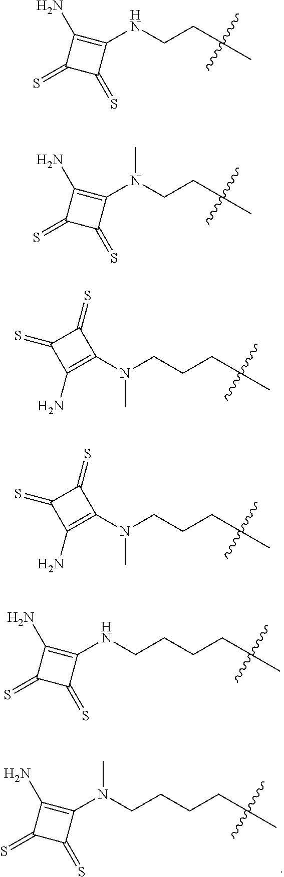

In some embodiments,

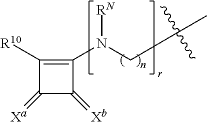

is selected from any of the following groups:

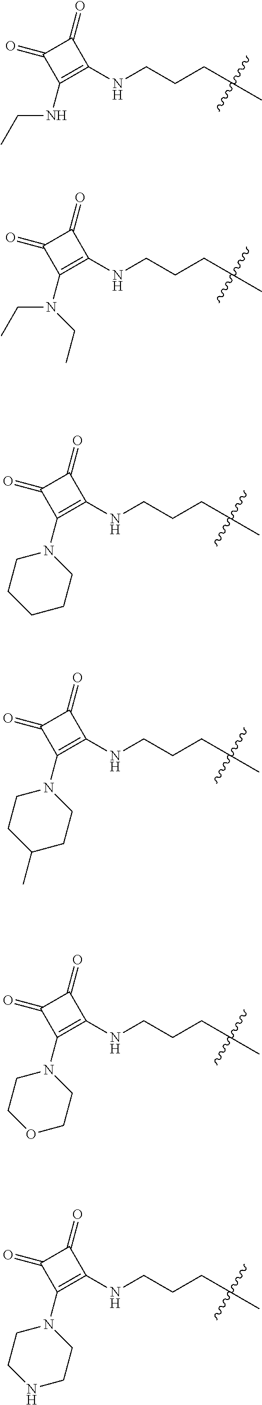 [SMILES: *CCCNc1c(NCC)c(=O)c1=O ; *CCCNc1c(N(CC)CC)c(=O)c1=O ; *CCCNc1c(N2CCCCC2)c(=O)c1=O ; *CCCNc1c(N2CCC(C)CC2)c(=O)c1=O ; *CCCNc1c(N2CCOCC2)c(=O)c1=O ; *CCCNc1c(N2CCNCC2)c(=O)c1=O]

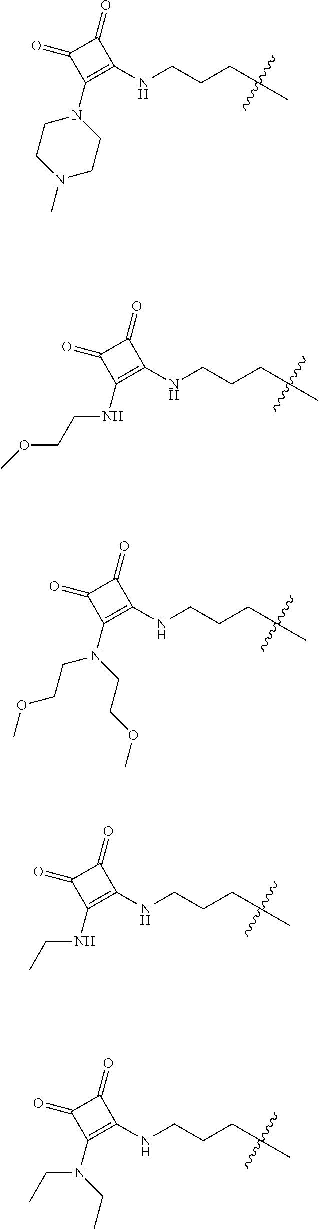 [SMILES: *CCCNc1c(N2CCN(C)CC2)c(=O)c1=O ; *CCCNc1c(NCCOC)c(=O)c1=O ; *CCCNc1c(N(CCOC)CCOC)c(=O)c1=O ; *CCCNc1c(NCC)c(=O)c1=O ; *CCCNc1c(N(CC)CC)c(=O)c1=O]

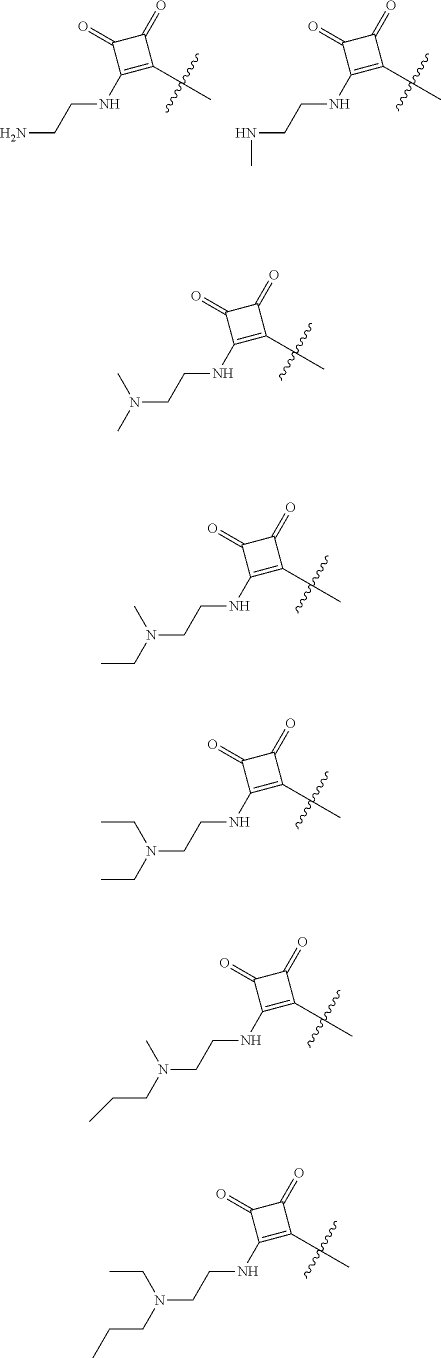

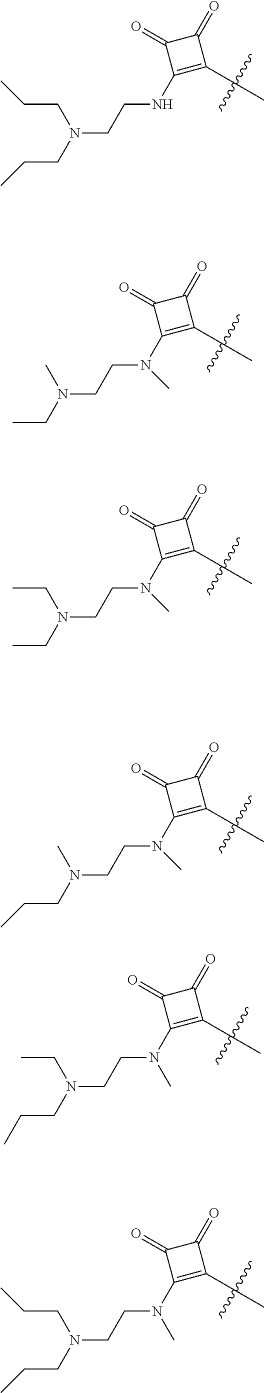 [SMILES: *c1c(NCCN(CCC)CCC)c(=O)c1=O ; *c1c(N(C)CCN(C)CC)c(=O)c1=O ; *c1c(N(C)CCN(CC)CC)c(=O)c1=O ; *c1c(N(C)CCN(C)CCC)c(=O)c1=O ; *c1c(N(C)CCN(CC)CCC)c(=O)c1=O ; *c1c(N(C)CCN(CCC)CCC)c(=O)c1=O]

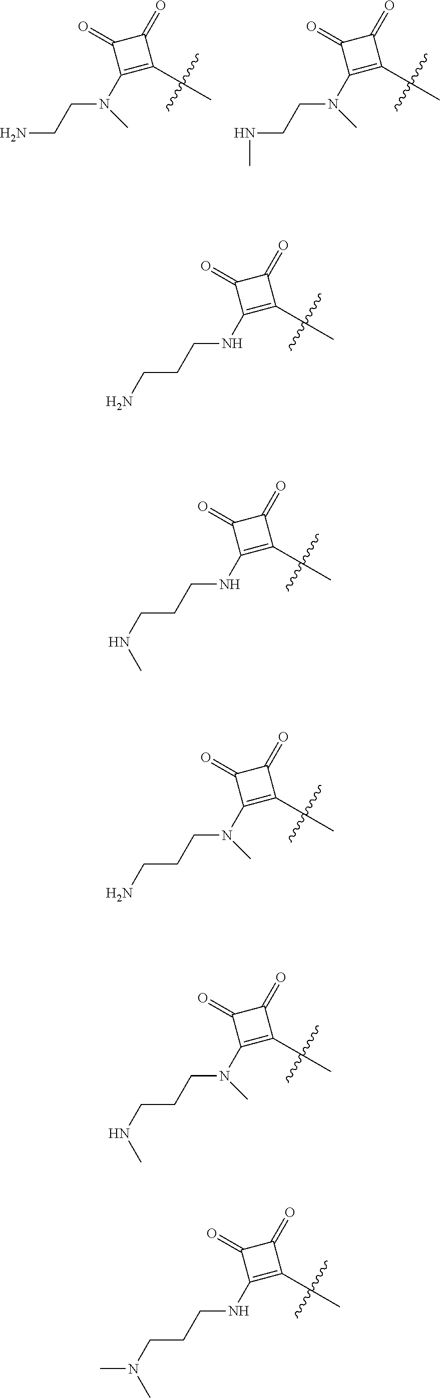

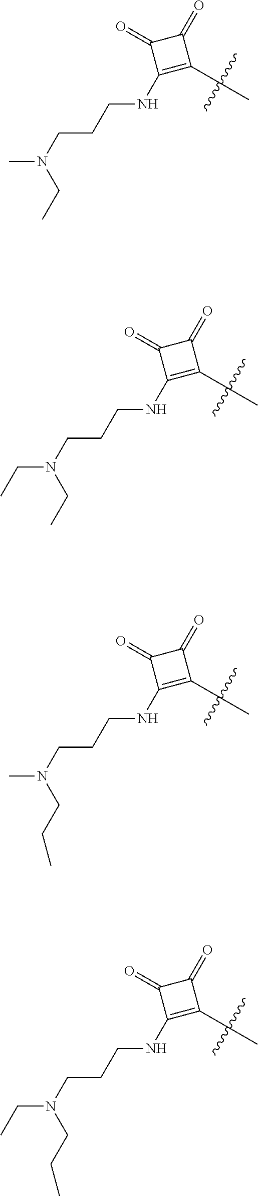 [SMILES: *c1c(NCCCN(C)CC)c(=O)c1=O ; *c1c(NCCCN(CC)CC)c(=O)c1=O ; *c1c(NCCCN(C)CCC)c(=O)c1=O ; *c1c(NCCCN(CC)CCC)c(=O)c1=O]

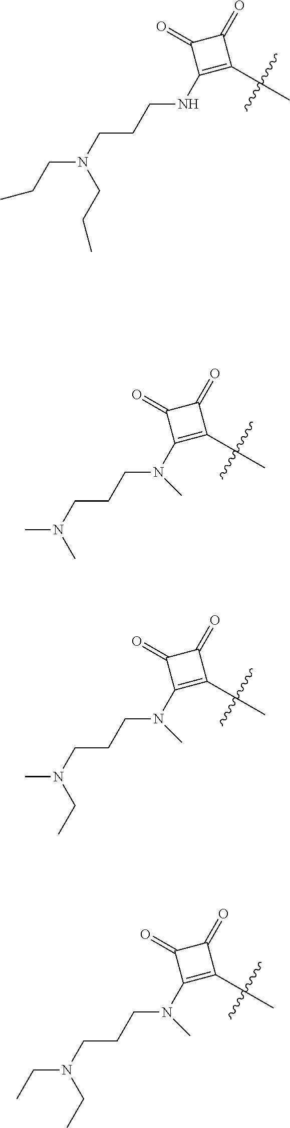 [SMILES: *c1c(NCCCN(CCC)CCC)c(=O)c1=O ; *c1c(N(C)CCCN(C)C)c(=O)c1=O ; *c1c(N(C)CCCN(C)CC)c(=O)c1=O ; *c1c(N(C)CCCN(CC)CC)c(=O)c1=O]

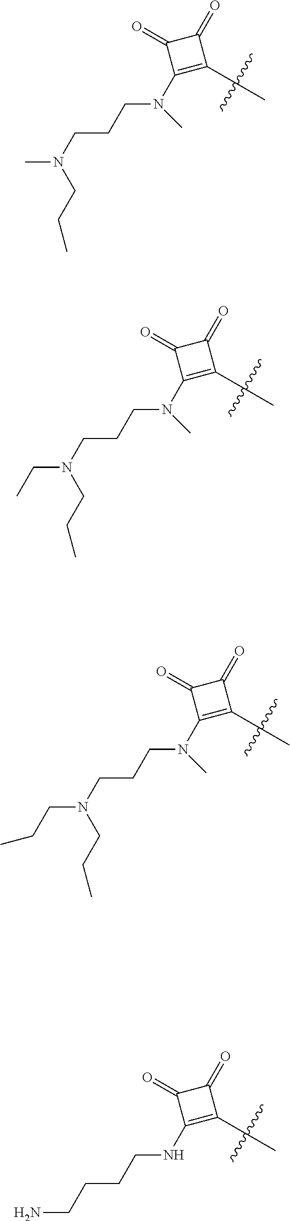 [SMILES: *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O ; *c1c(NCCCCN)c(=O)c1=O]

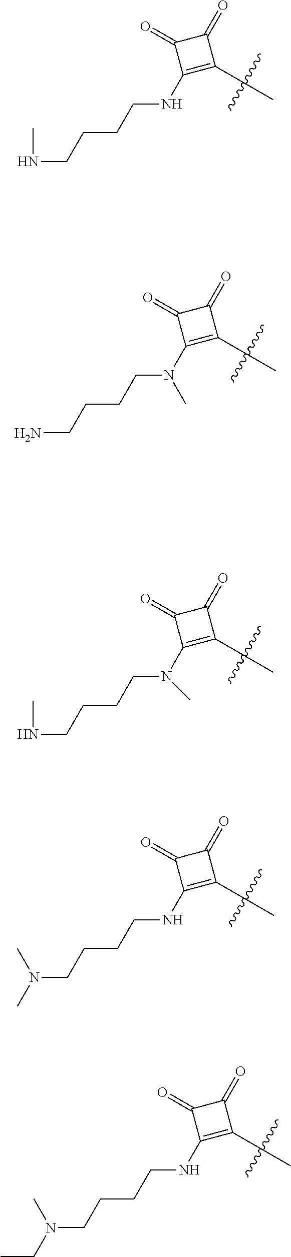 [SMILES: *c1c(NCCCCNC)c(=O)c1=O ; *c1c(N(C)CCCCN)c(=O)c1=O ; *c1c(N(C)CCCCNC)c(=O)c1=O ; *c1c(NCCCCN(C)C)c(=O)c1=O ; *c1c(NCCCCN(C)CC)c(=O)c1=O]

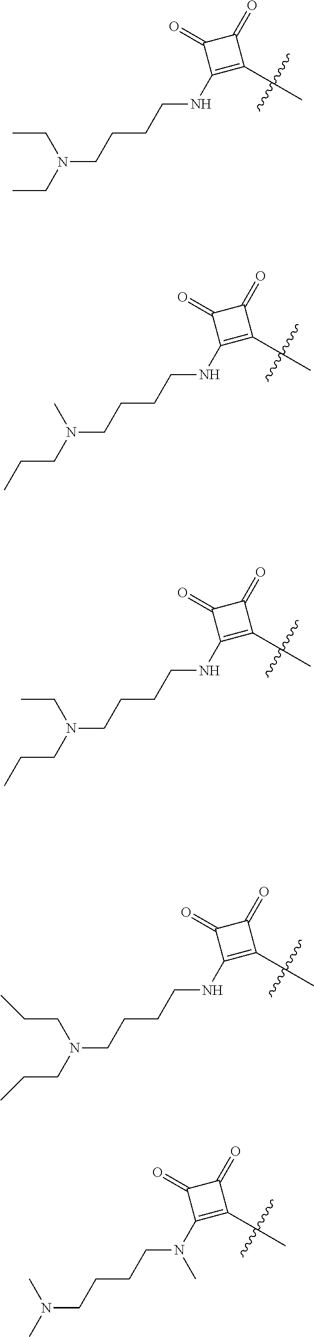 [SMILES: *c1c(NCCCCN(CC)CC)c(=O)c1=O ; *c1c(NCCCCN(C)CCC)c(=O)c1=O ; *c1c(NCCCCN(CC)CCC)c(=O)c1=O ; *c1c(NCCCCN(CCC)CCC)c(=O)c1=O ; *c1c(N(C)CCCCN(C)C)c(=O)c1=O]

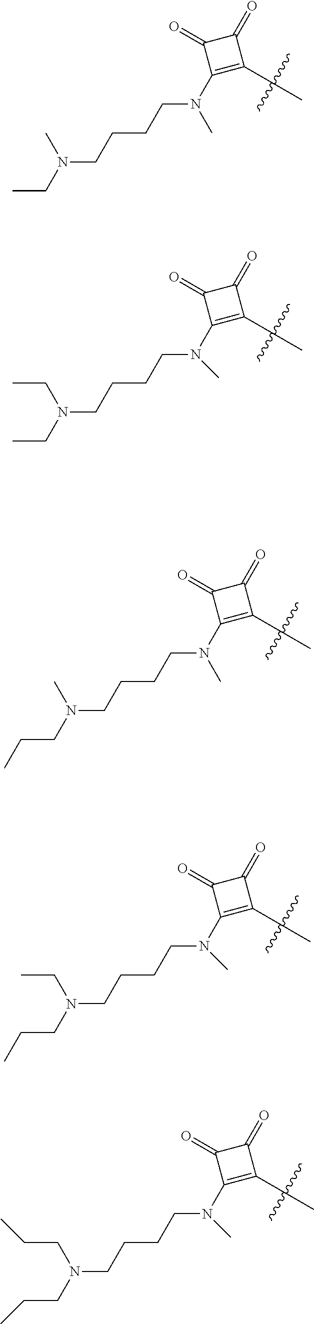 [SMILES: *c1c(N(C)CCCCN(C)CC)c(=O)c1=O ; *c1c(N(C)CCCCN(CC)CC)c(=O)c1=O ; *c1c(N(C)CCCCN(C)CCC)c(=O)c1=O ; *c1c(N(C)CCCCN(CC)CCC)c(=O)c1=O ; *c1c(N(C)CCCCN(CCC)CCC)c(=O)c1=O]

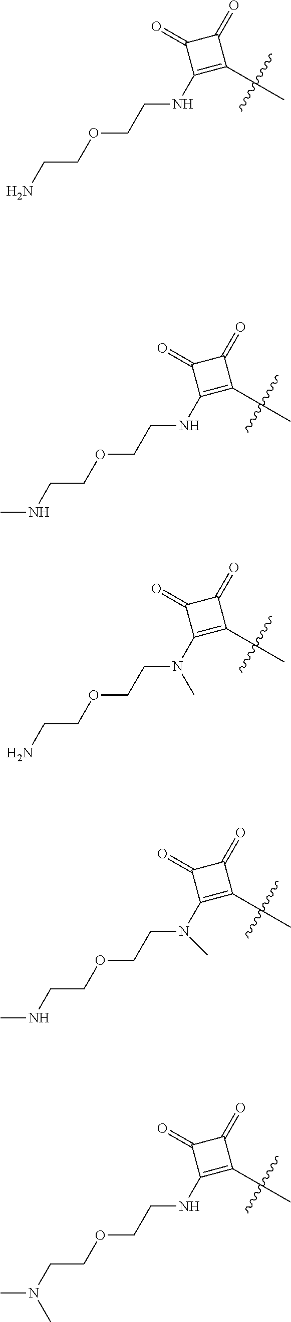 [SMILES: *c1c(NCCOCCN)c(=O)c1=O ; *c1c(NCCOCCNC)c(=O)c1=O ; *c1c(N(C)CCOCCN)c(=O)c1=O ; *c1c(N(C)CCOCCNC)c(=O)c1=O ; *c1c(NCCOCCN(C)C)c(=O)c1=O]

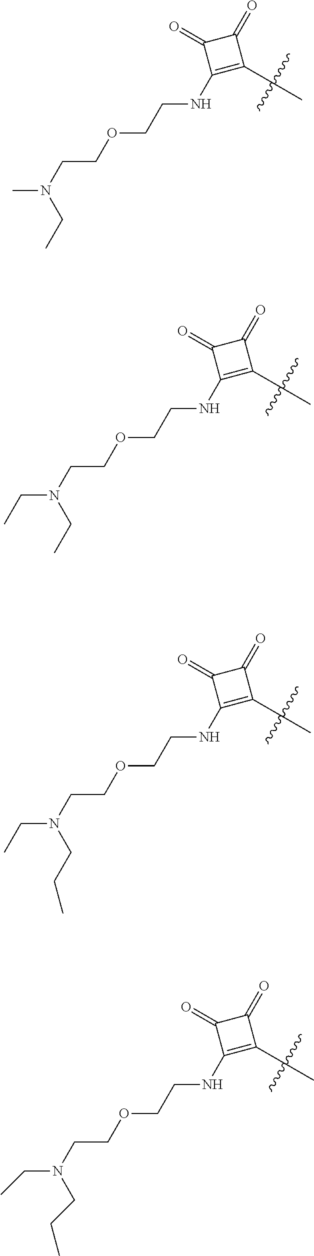 [SMILES: *c1c(NCCOCCN(C)CC)c(=O)c1=O ; *c1c(NCCOCCN(CC)CC)c(=O)c1=O ; *c1c(NCCOCCN(CC)CCC)c(=O)c1=O ; *c1c(NCCOCCN(CC)CCC)c(=O)c1=O]

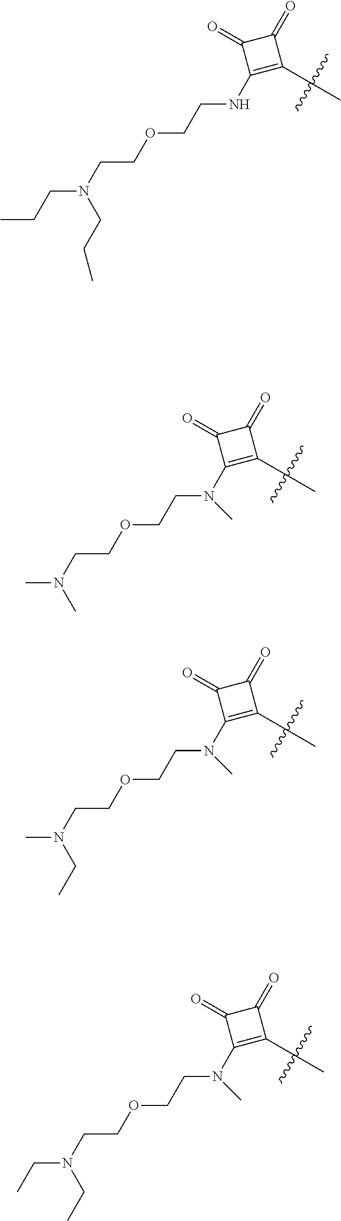 [SMILES: *c1c(NCCOCCN(CCC)CCC)c(=O)c1=O ; *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O]

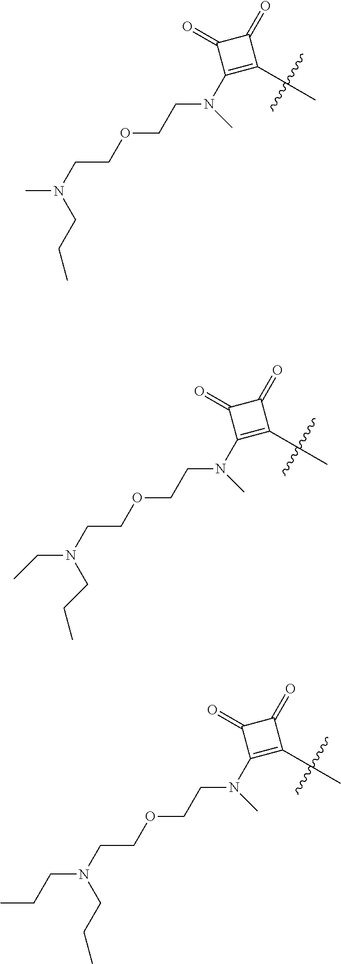 [SMILES: *c1c(N(C)CCOCCN(C)CCC)c(=O)c1=O ; *c1c(N(C)CCOCCN(CC)CCC)c(=O)c1=O ; *c1c(N(C)CCOCCN(CCC)CCC)c(=O)c1=O]

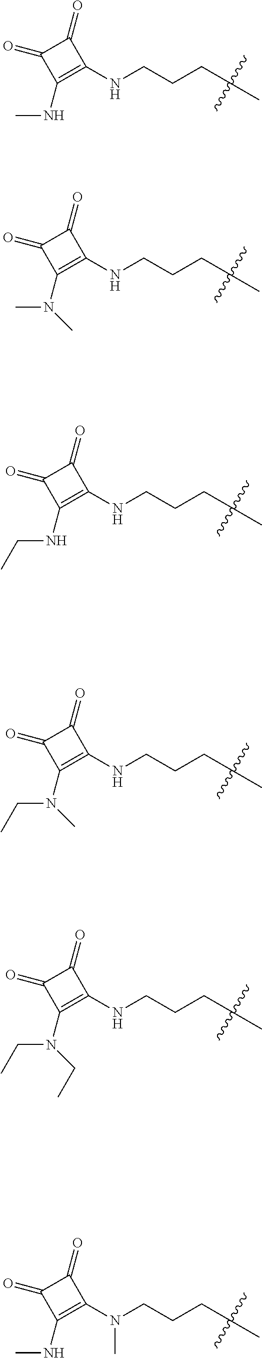 [SMILES: *CCCNc1c(NC)c(=O)c1=O ; *CCCNc1c(N(C)C)c(=O)c1=O ; *CCCNc1c(NCC)c(=O)c1=O ; *CCCNc1c(N(C)CC)c(=O)c1=O ; *CCCNc1c(N(CC)CC)c(=O)c1=O ; *CCCN(C)c1c(NC)c(=O)c1=O]

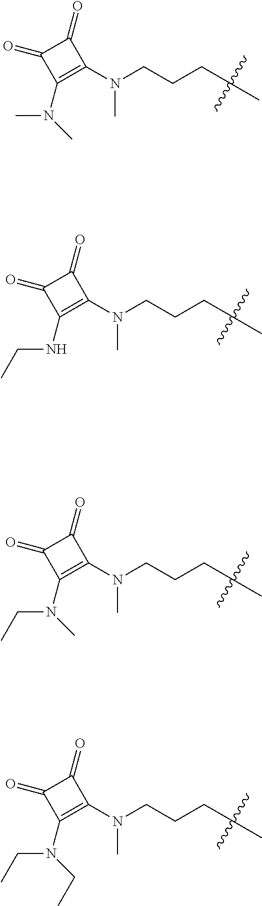 [SMILES: *CCCN(C)c1c(N(C)C)c(=O)c1=O ; *CCCN(C)c1c(NCC)c(=O)c1=O ; *CCCN(C)c1c(N(C)CC)c(=O)c1=O ; *CCCN(C)c1c(N(CC)CC)c(=O)c1=O]

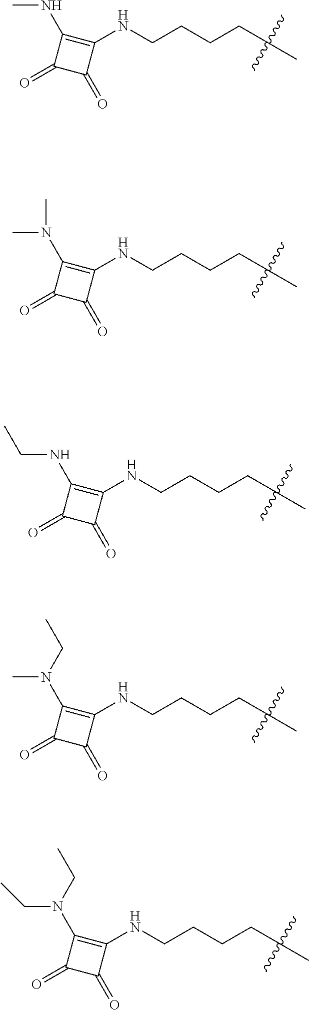 [SMILES: *CCCCNc1c(NC)c(=O)c1=O ; *CCCCNc1c(N(C)C)c(=O)c1=O ; *CCCCNc1c(NCC)c(=O)c1=O ; *CCCCNc1c(N(C)CC)c(=O)c1=O ; *CCCCNc1c(N(CC)CC)c(=O)c1=O]

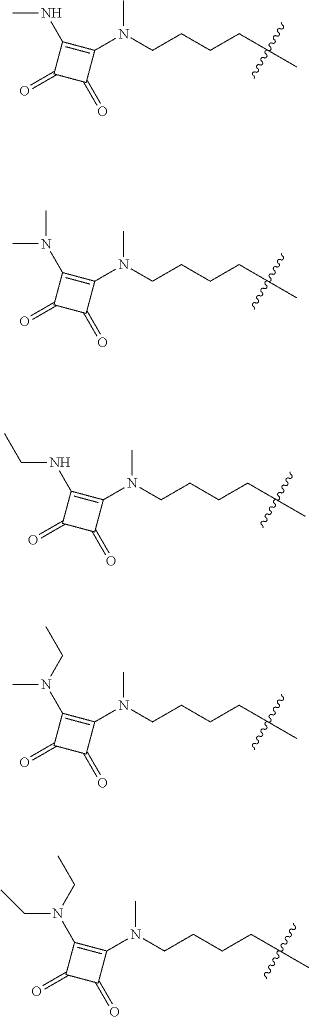 [SMILES: *CCCCN(C)c1c(NC)c(=O)c1=O ; *CCCCN(C)c1c(N(C)C)c(=O)c1=O ; *CCCCN(C)c1c(NCC)c(=O)c1=O ; *CCCCN(C)c1c(N(C)CC)c(=O)c1=O ; *CCCCN(C)c1c(N(CC)CC)c(=O)c1=O]

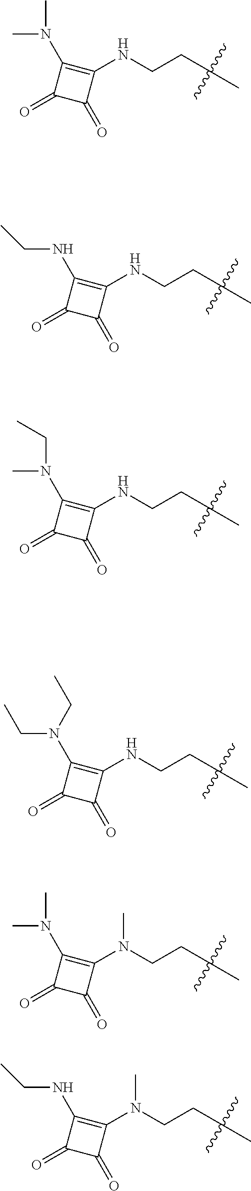 [SMILES: *CCNc1c(N(C)C)c(=O)c1=O ; *CCNc1c(NCC)c(=O)c1=O ; *CCNc1c(N(C)CC)c(=O)c1=O ; *CCNc1c(N(CC)CC)c(=O)c1=O ; *CCN(C)c1c(N(C)C)c(=O)c1=O ; *CCN(C)c1c(NCC)c(=O)c1=O]

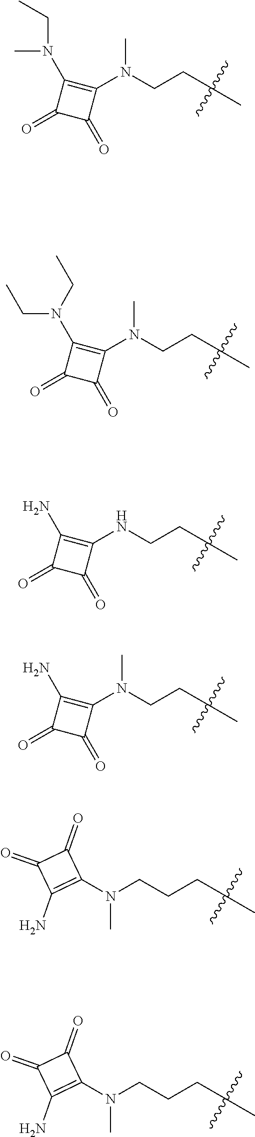 [SMILES: *CCN(C)c1c(N(C)CC)c(=O)c1=O ; *CCN(C)c1c(N(CC)CC)c(=O)c1=O ; *CCNc1c(N)c(=O)c1=O ; *CCN(C)c1c(N)c(=O)c1=O ; *CCCN(C)c1c(N)c(=O)c1=O ; *CCCN(C)c1c(N)c(=O)c1=O]

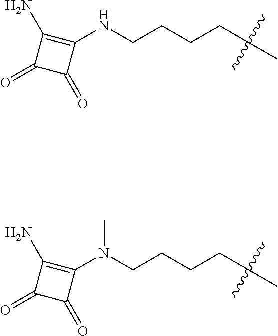 [SMILES: *CCCCNc1c(N)c(=O)c1=O ; *CCCCN(C)c1c(N)c(=O)c1=O]

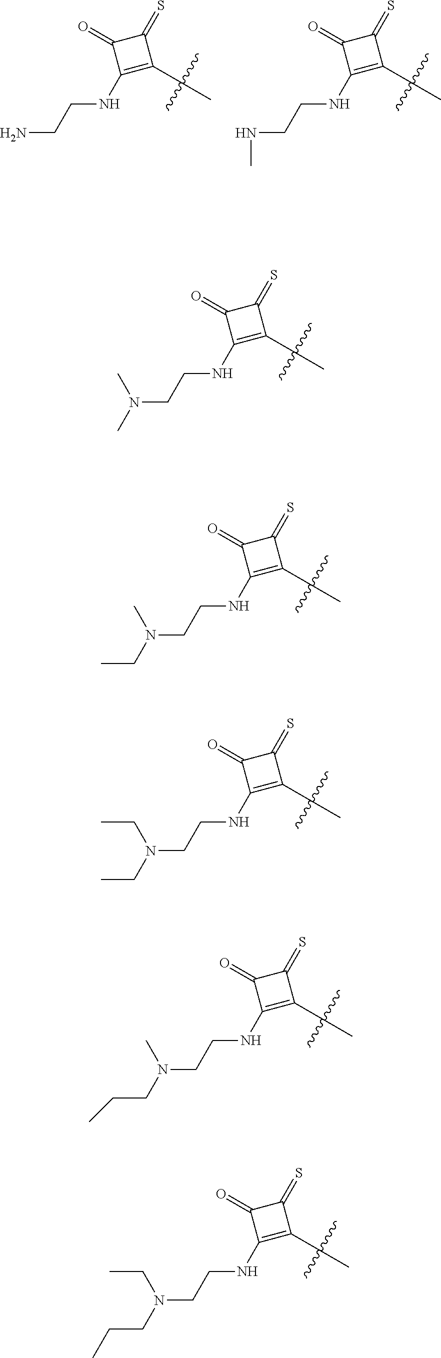

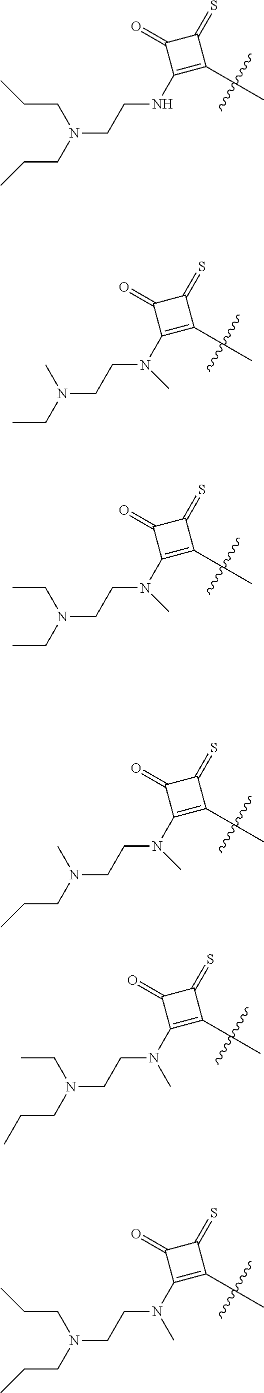 [SMILES: *c1c(NCCN(CCC)CCC)c(=O)c1=S ; *c1c(N(C)CCN(C)CC)c(=O)c1=S ; *c1c(N(C)CCN(CC)CC)c(=O)c1=S ; *c1c(N(C)CCN(C)CCC)c(=O)c1=S ; *c1c(N(C)CCN(CC)CCC)c(=O)c1=S ; *c1c(N(C)CCN(CCC)CCC)c(=O)c1=S]

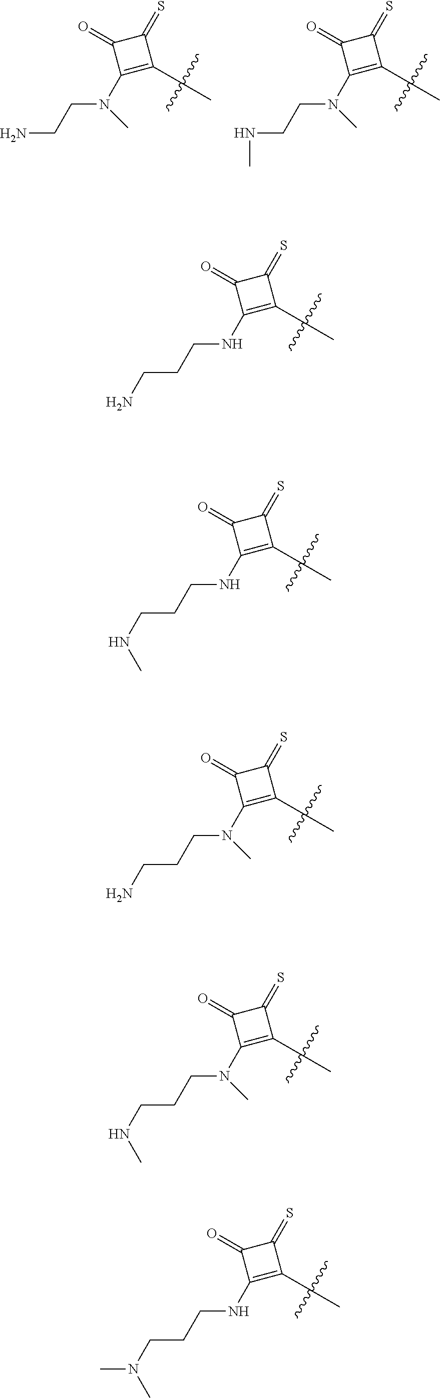

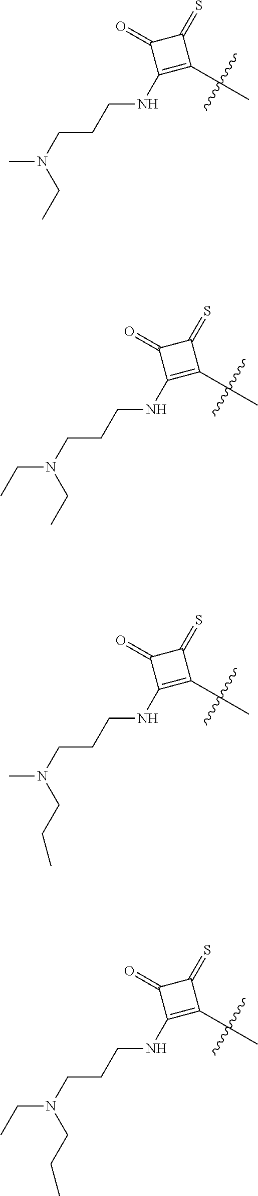 [SMILES: *c1c(NCCCN(C)CC)c(=O)c1=S ; *c1c(NCCCN(CC)CC)c(=O)c1=S ; *c1c(NCCCN(C)CCC)c(=O)c1=S ; *c1c(NCCCN(CC)CCC)c(=O)c1=S]

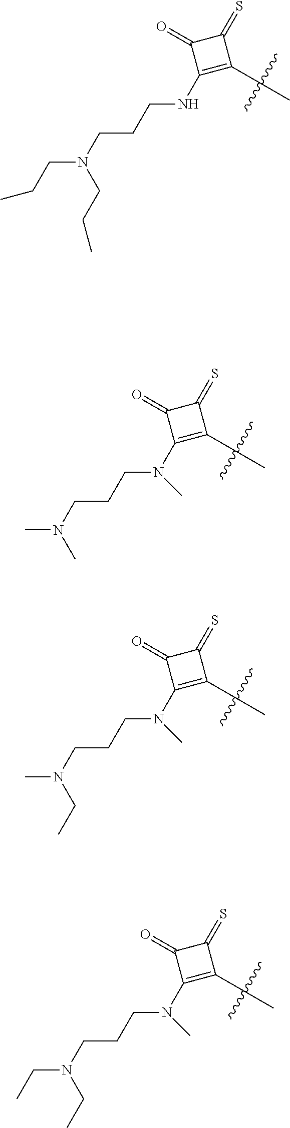 [SMILES: *c1c(NCCCN(CCC)CCC)c(=O)c1=S ; *c1c(N(C)CCCN(C)C)c(=O)c1=S ; *c1c(N(C)CCCN(C)CC)c(=O)c1=S ; *c1c(N(C)CCCN(CC)CC)c(=O)c1=S]

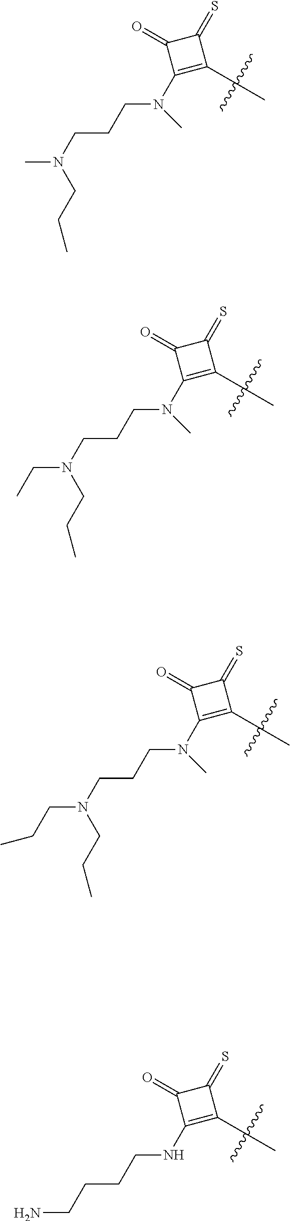 [SMILES: *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S ; *c1c(NCCCCN)c(=O)c1=S]

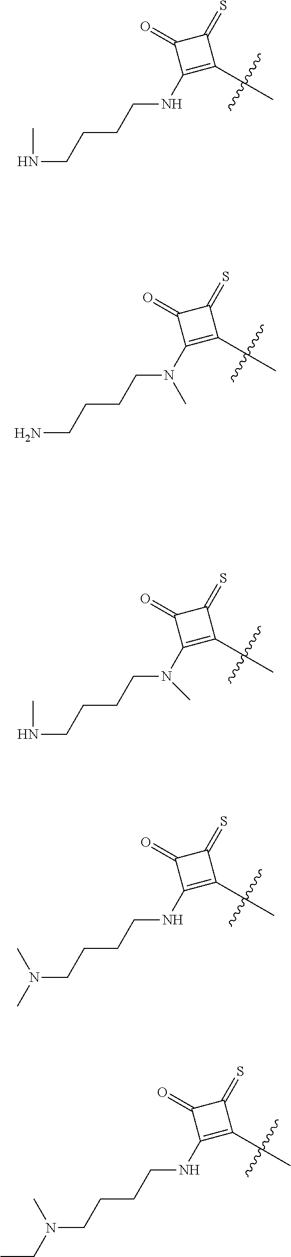 [SMILES: *c1c(NCCCCNC)c(=O)c1=S ; *c1c(N(C)CCCCN)c(=O)c1=S ; *c1c(N(C)CCCCNC)c(=O)c1=S ; *c1c(NCCCCN(C)C)c(=O)c1=S ; *c1c(NCCCCN(C)CC)c(=O)c1=S]

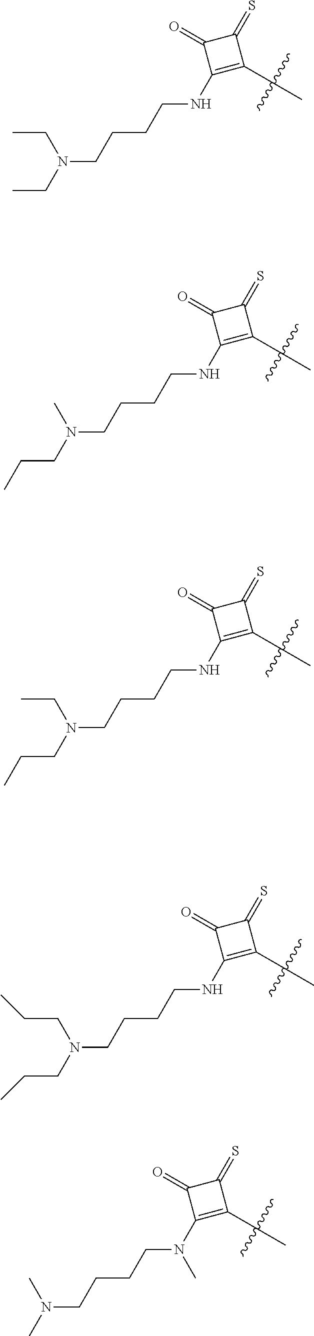 [SMILES: *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S ; *c1c(N(C)CCCCN(C)C)c(=O)c1=S]

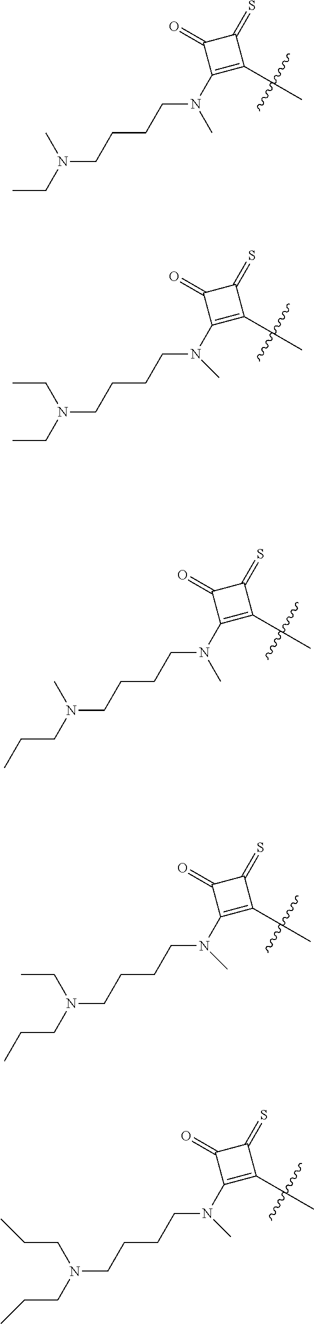 [SMILES: *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

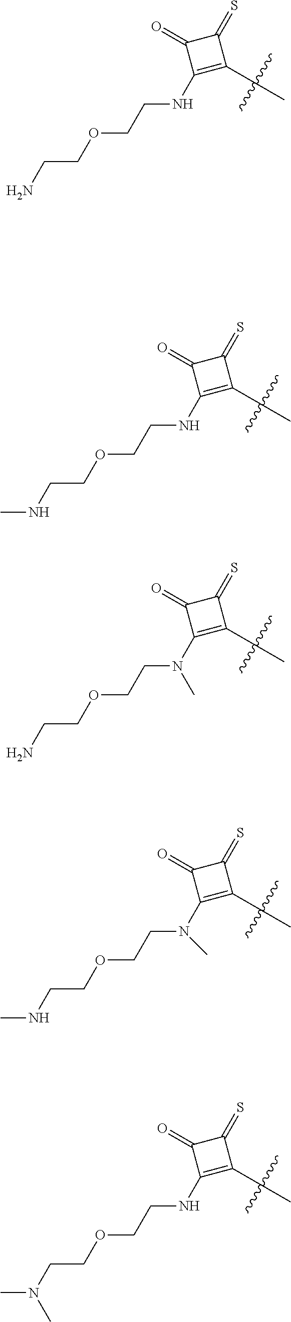 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S ; *c1c(NCCOCCN(C)C)c(=O)c1=S]

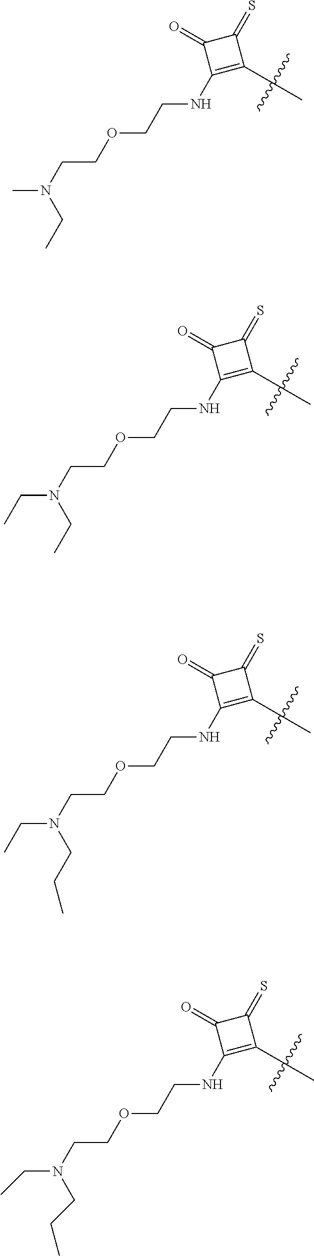 [SMILES: *c1c(NCCOCCN(C)CC)c(=O)c1=S ; *c1c(NCCOCCN(CC)CC)c(=O)c1=S ; *c1c(NCCOCCN(CC)CCC)c(=O)c1=S ; *c1c(NCCOCCN(CC)CCC)c(=O)c1=S]

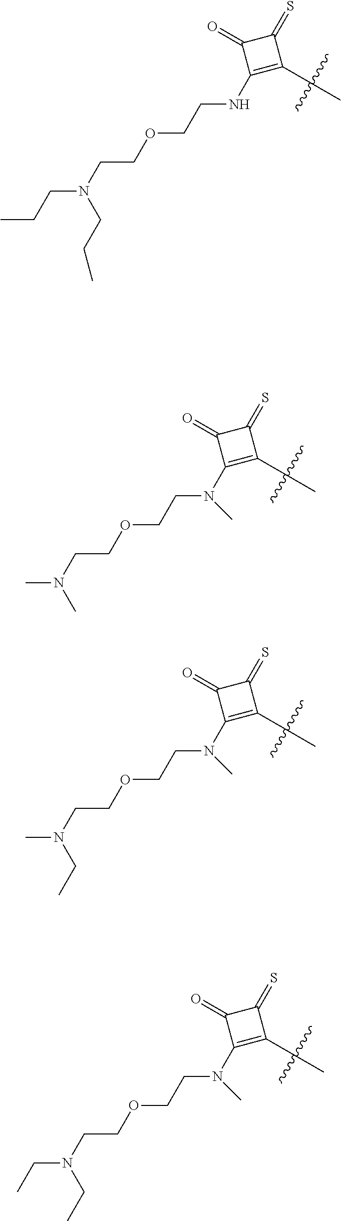 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S]

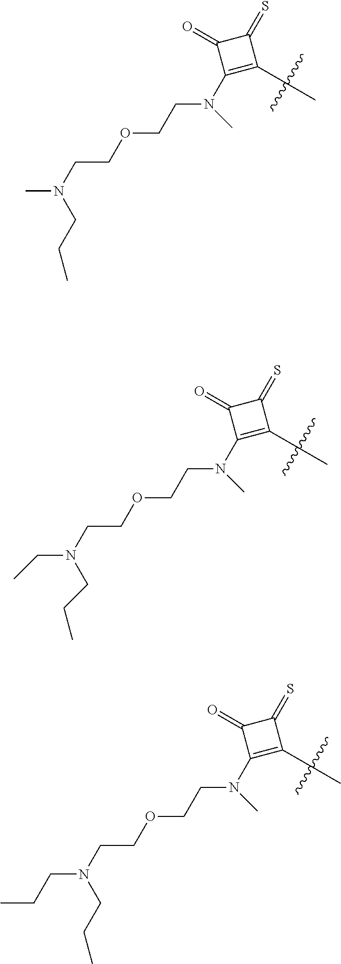 [SMILES: *c1c(N(C)CCOCCN(C)CCC)c(=O)c1=S ; *c1c(N(C)CCOCCN(CC)CCC)c(=O)c1=S ; *c1c(N(C)CCOCCN(CCC)CCC)c(=O)c1=S]

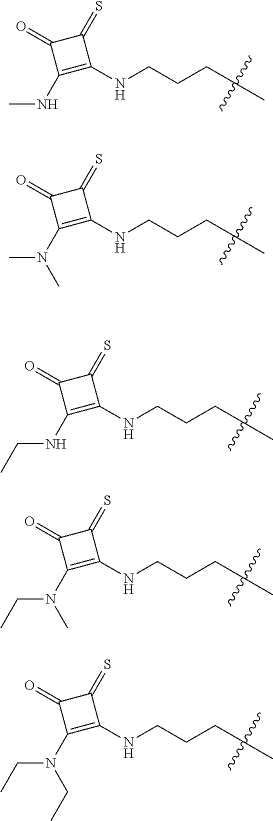 [SMILES: *CCCNc1c(NC)c(=O)c1=S ; *CCCNc1c(N(C)C)c(=O)c1=S ; *CCCNc1c(NCC)c(=O)c1=S ; *CCCNc1c(N(C)CC)c(=O)c1=S ; *CCCNc1c(N(CC)CC)c(=O)c1=S]

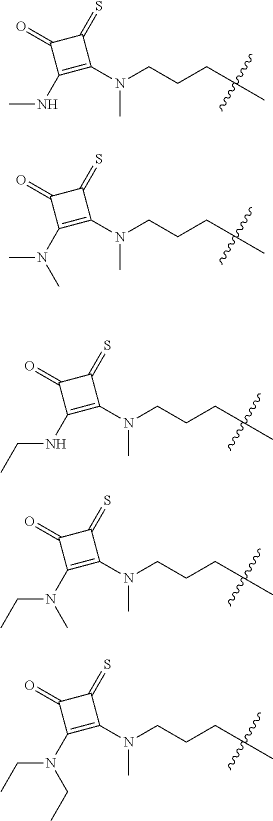 [SMILES: *CCCN(C)c1c(NC)c(=O)c1=S ; *CCCN(C)c1c(N(C)C)c(=O)c1=S ; *CCCN(C)c1c(NCC)c(=O)c1=S ; *CCCN(C)c1c(N(C)CC)c(=O)c1=S ; *CCCN(C)c1c(N(CC)CC)c(=O)c1=S]

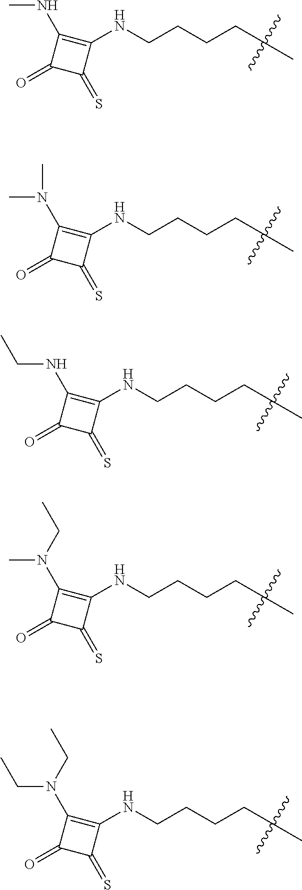 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

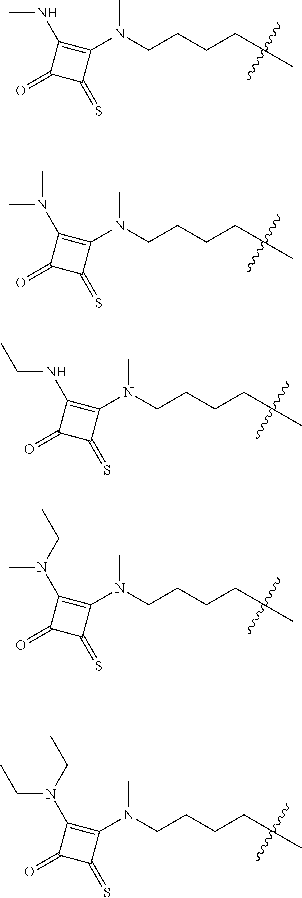 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

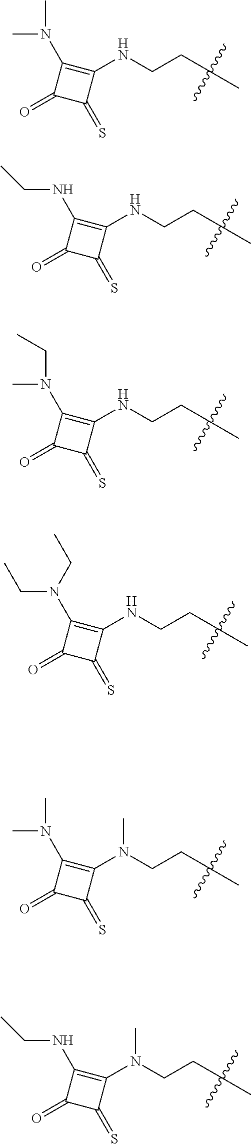 [SMILES: *CCNc1c(N(C)C)c(=O)c1=S ; *CCNc1c(NCC)c(=O)c1=S ; *CCNc1c(N(C)CC)c(=O)c1=S ; *CCNc1c(N(CC)CC)c(=O)c1=S ; *CCN(C)c1c(N(C)C)c(=O)c1=S ; *CCN(C)c1c(NCC)c(=O)c1=S]

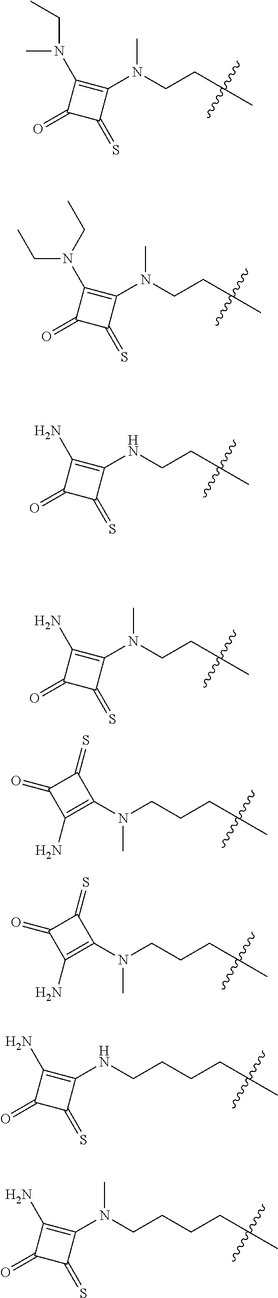

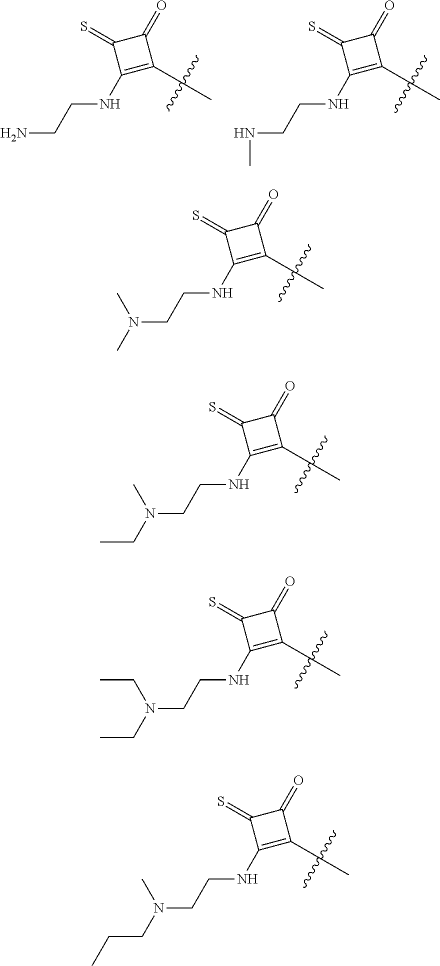

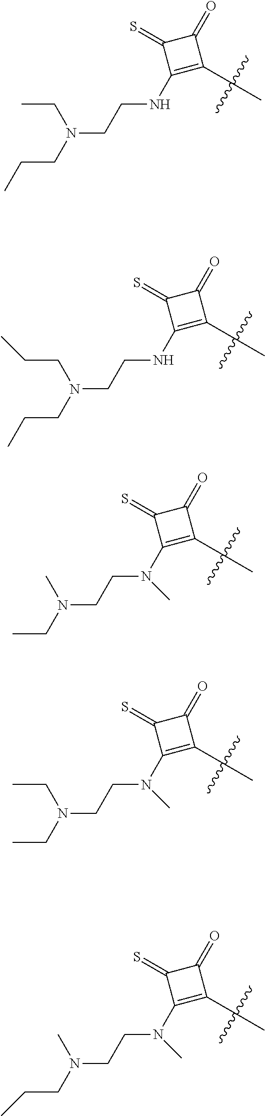 [SMILES: *c1c(NCCN(CC)CCC)c(=S)c1=O ; *c1c(NCCN(CCC)CCC)c(=S)c1=O ; *c1c(N(C)CCN(C)CC)c(=S)c1=O ; *c1c(N(C)CCN(CC)CC)c(=S)c1=O ; *c1c(N(C)CCN(C)CCC)c(=S)c1=O]

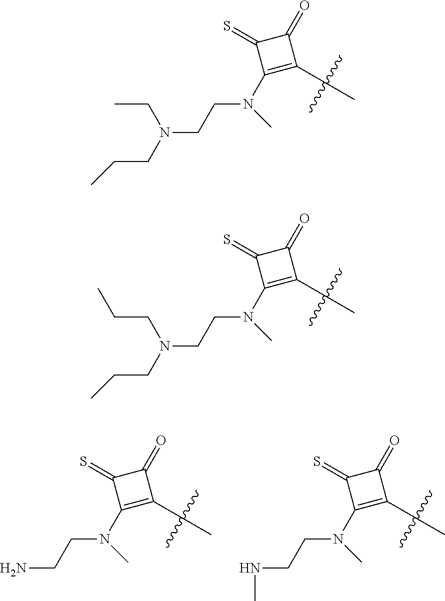 [SMILES: *c1c(N(C)CCN(CC)CCC)c(=S)c1=O ; *c1c(N(C)CCN(CCC)CCC)c(=S)c1=O ; *c1c(N(C)CCN)c(=S)c1=O ; *c1c(N(C)CCNC)c(=S)c1=O]

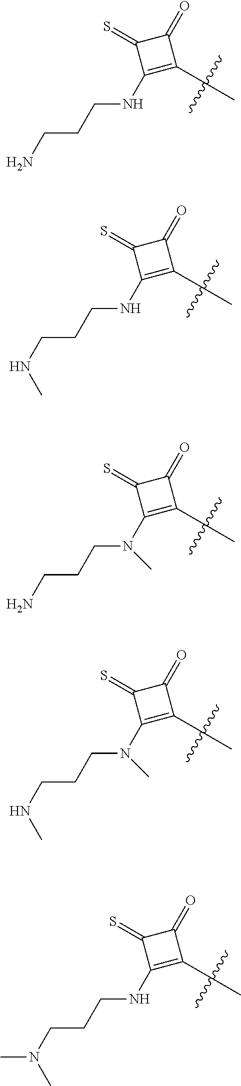 [SMILES: *c1c(NCCCN)c(=S)c1=O ; *c1c(NCCCNC)c(=S)c1=O ; *c1c(N(C)CCCN)c(=S)c1=O ; *c1c(N(C)CCCNC)c(=S)c1=O ; *c1c(NCCCN(C)C)c(=S)c1=O]

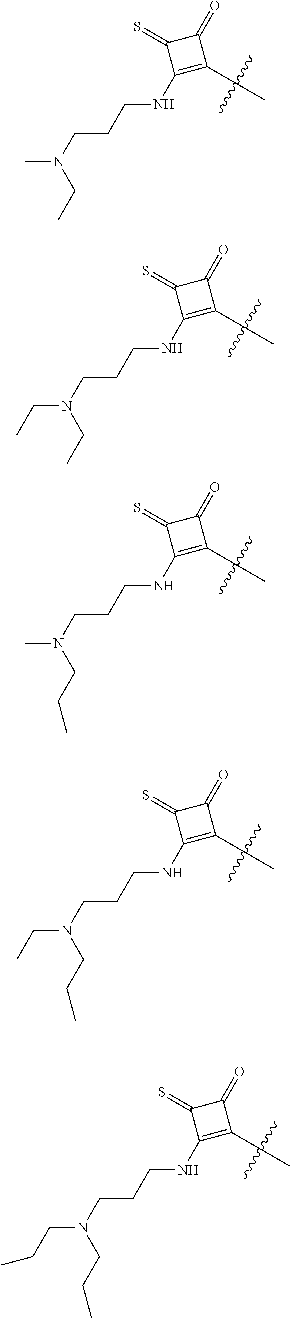 [SMILES: *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O ; *c1c(NCCCN(CCC)CCC)c(=S)c1=O]

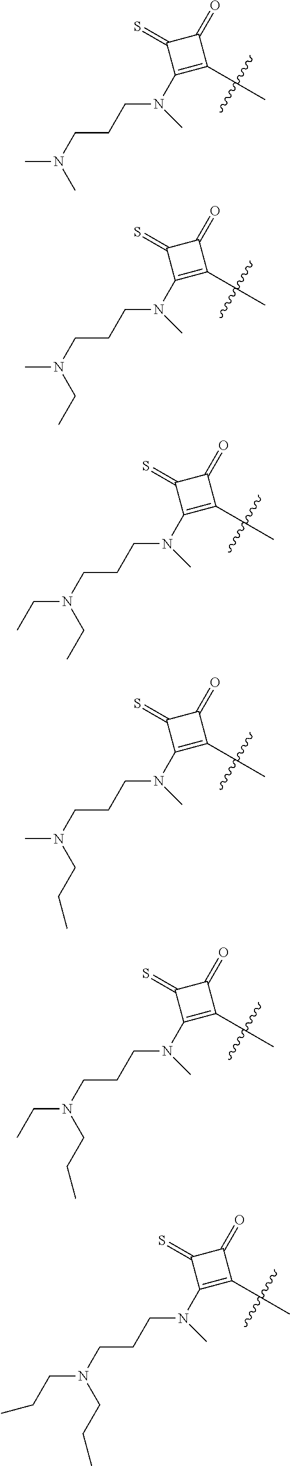 [SMILES: *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O ; *c1c(N(C)CCCN(CCC)CCC)c(=S)c1=O]

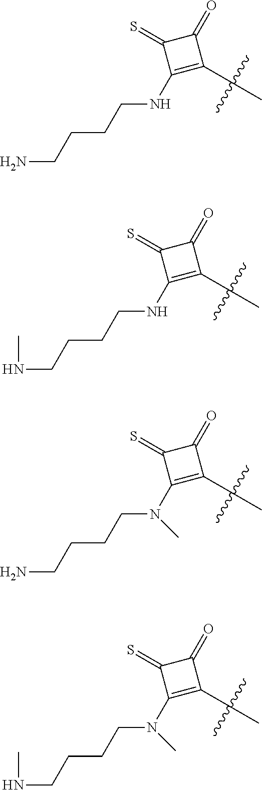 [SMILES: *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O]

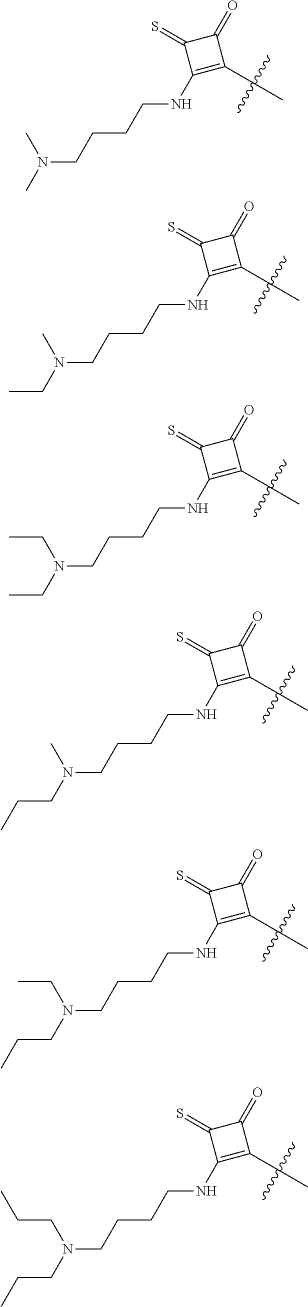 [SMILES: *c1c(NCCCCN(C)C)c(=S)c1=O ; *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O]

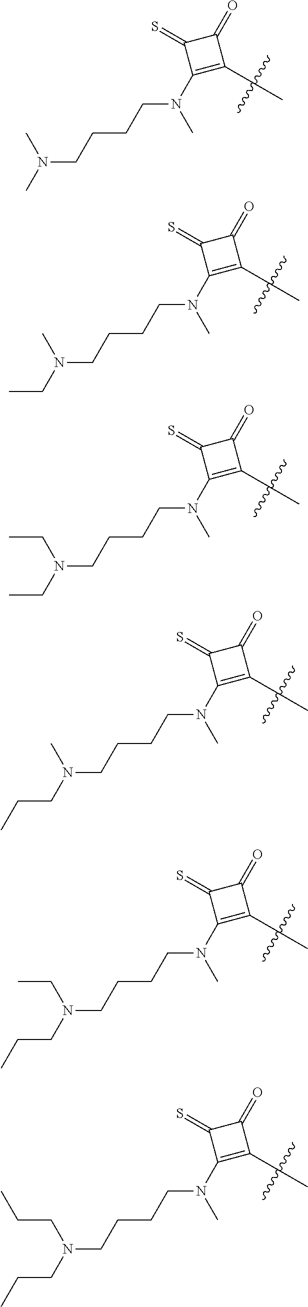 [SMILES: *c1c(N(C)CCCCN(C)C)c(=S)c1=O ; *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O]

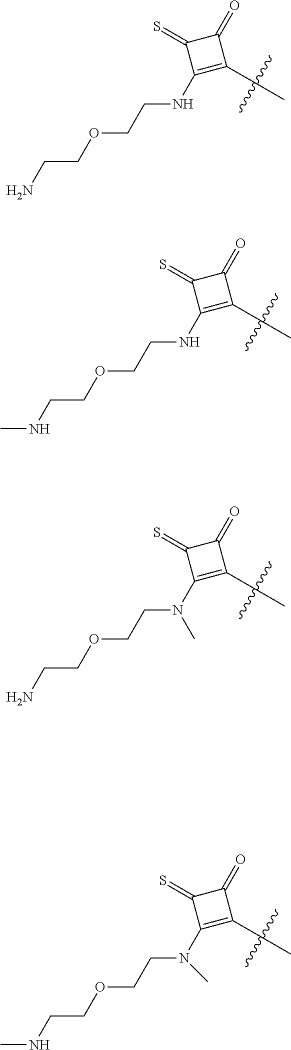 [SMILES: *c1c(NCCOCCN)c(=S)c1=O ; *c1c(NCCOCCNC)c(=S)c1=O ; *c1c(N(C)CCOCCN)c(=S)c1=O ; *c1c(N(C)CCOCCNC)c(=S)c1=O]

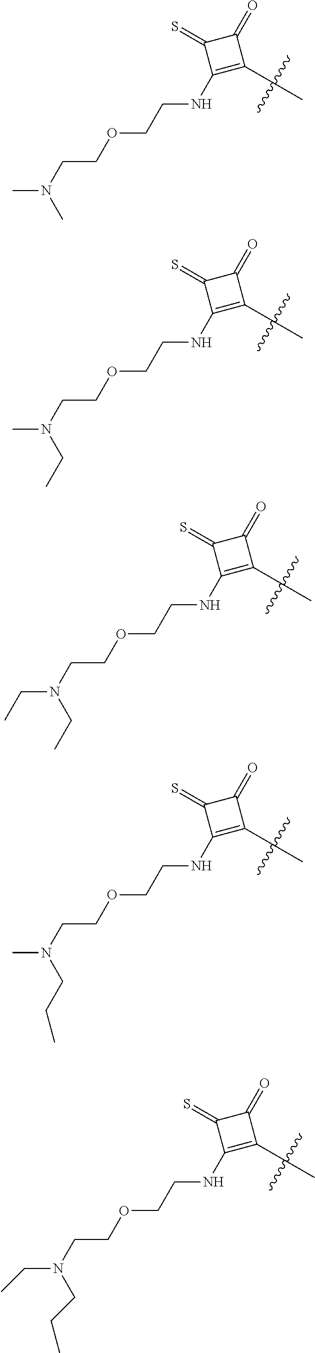 [SMILES: *c1c(NCCOCCN(C)C)c(=S)c1=O ; *c1c(NCCOCCN(C)CC)c(=S)c1=O ; *c1c(NCCOCCN(CC)CC)c(=S)c1=O ; *c1c(NCCOCCN(C)CCC)c(=S)c1=O ; *c1c(NCCOCCN(CC)CCC)c(=S)c1=O]

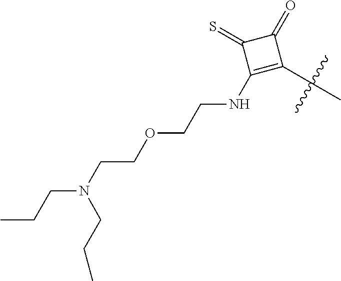 [SMILES: *c1c(NCCOCCN(CCC)CCC)c(=S)c1=O]

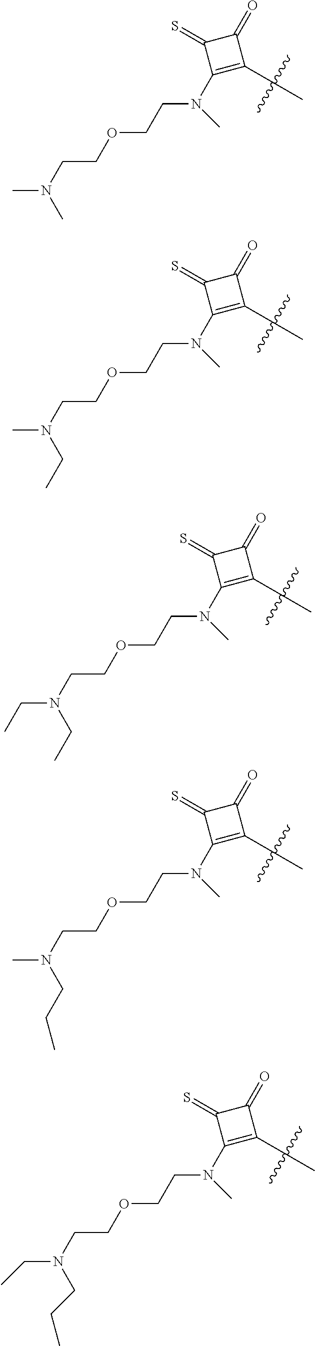 [SMILES: *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O]

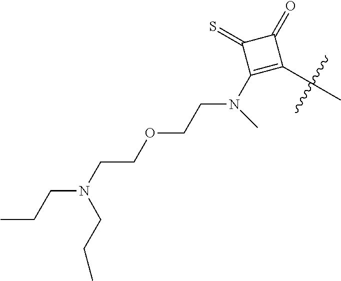 [SMILES: *c1c(N(C)CCOCCN(CCC)CCC)c(=S)c1=O]

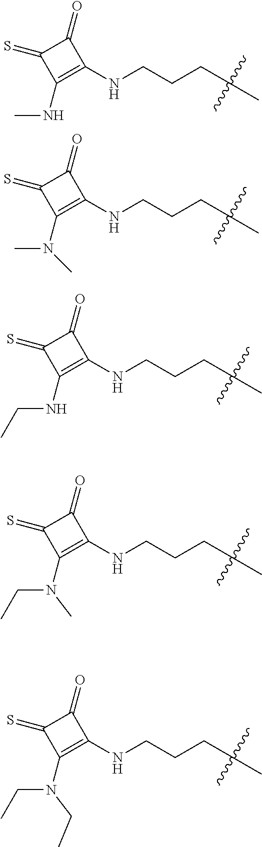

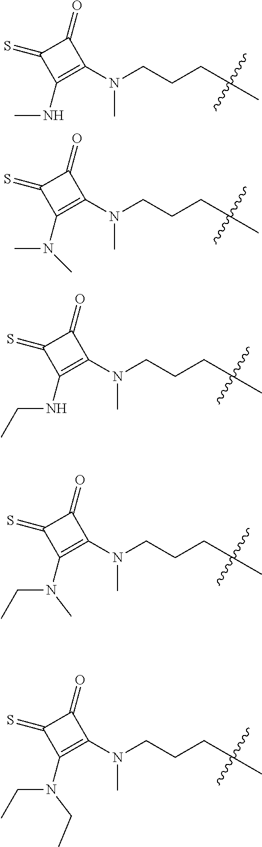

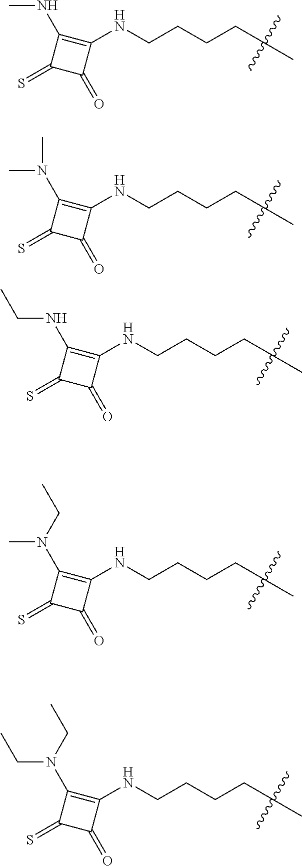

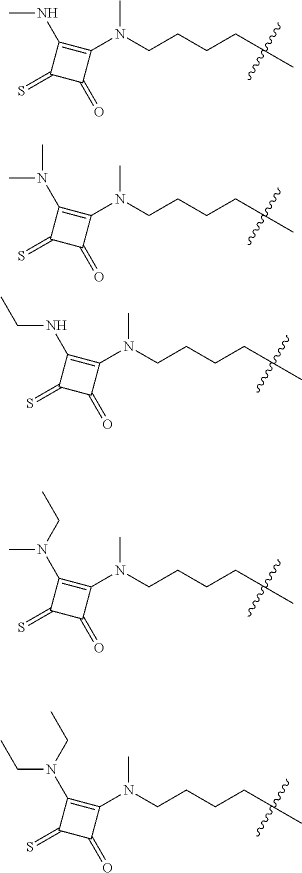

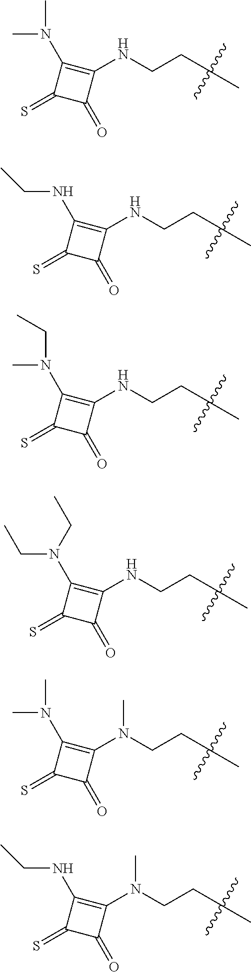 [SMILES: *CCNc1c(N(C)C)c(=S)c1=O ; *CCNc1c(NCC)c(=S)c1=O ; *CCNc1c(N(C)CC)c(=S)c1=O ; *CCNc1c(N(CC)CC)c(=S)c1=O ; *CCN(C)c1c(N(C)C)c(=S)c1=O ; *CCN(C)c1c(NCC)c(=S)c1=O]

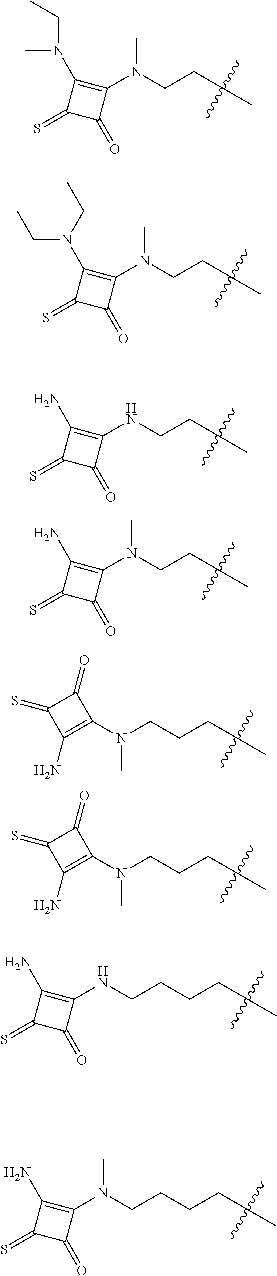

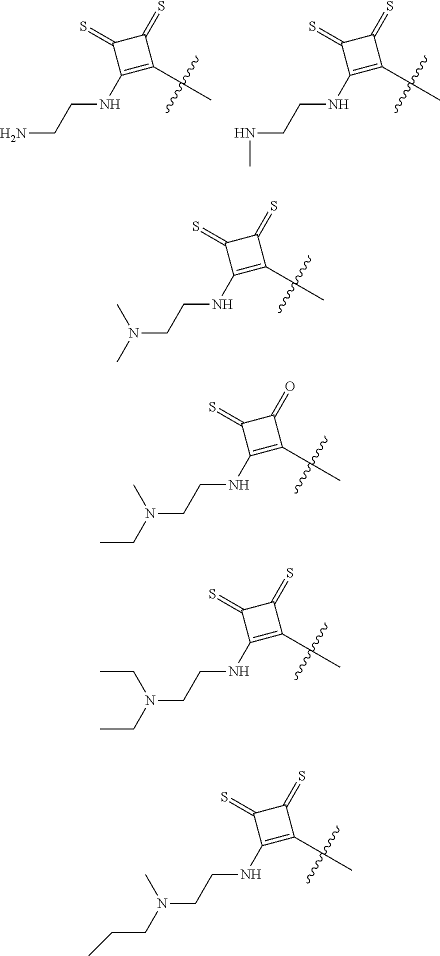

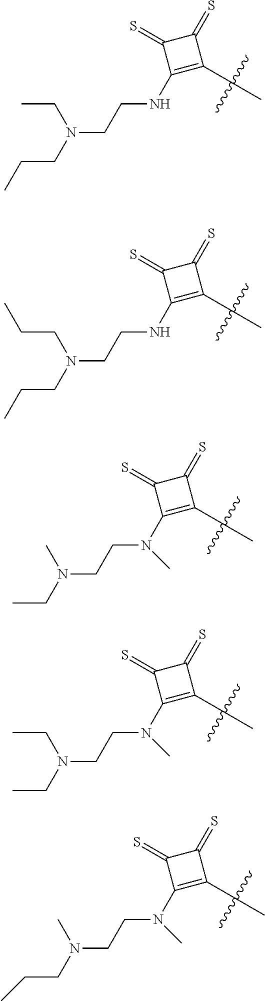 [SMILES: *c1c(NCCN(CC)CCC)c(=S)c1=S ; *c1c(NCCN(CCC)CCC)c(=S)c1=S ; *c1c(N(C)CCN(C)CC)c(=S)c1=S ; *c1c(N(C)CCN(CC)CC)c(=S)c1=S ; *c1c(N(C)CCN(C)CCC)c(=S)c1=S]

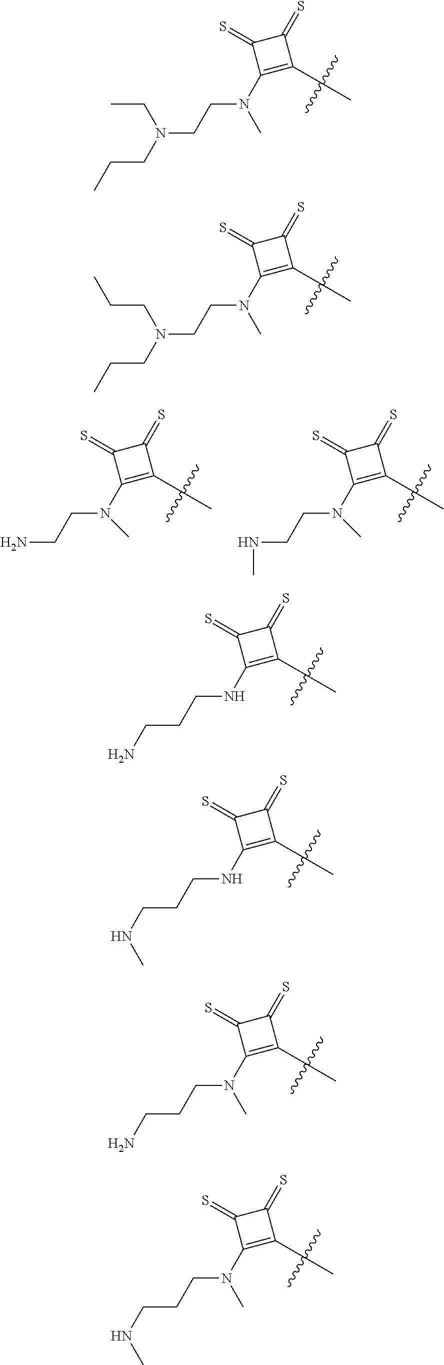

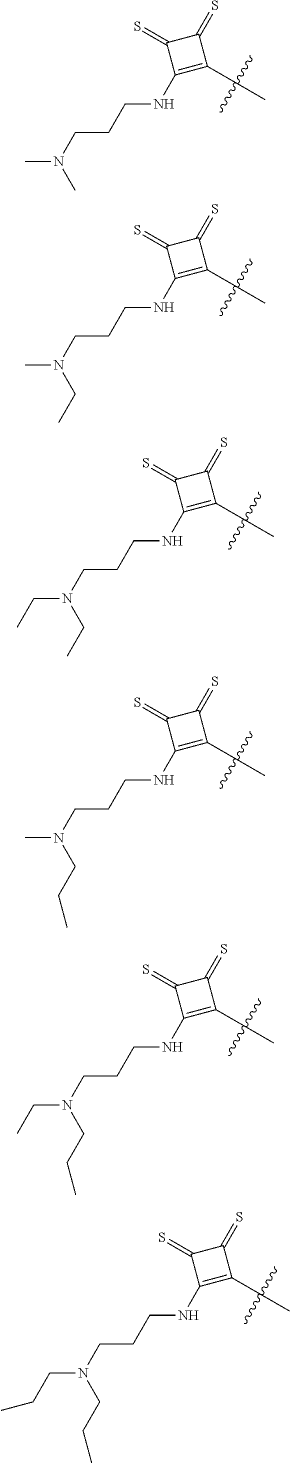 [SMILES: *c1c(NCCCN(C)C)c(=S)c1=S ; *c1c(NCCCN(C)CC)c(=S)c1=S ; *c1c(NCCCN(CC)CC)c(=S)c1=S ; *c1c(NCCCN(C)CCC)c(=S)c1=S ; *c1c(NCCCN(CC)CCC)c(=S)c1=S ; *c1c(NCCCN(CCC)CCC)c(=S)c1=S]

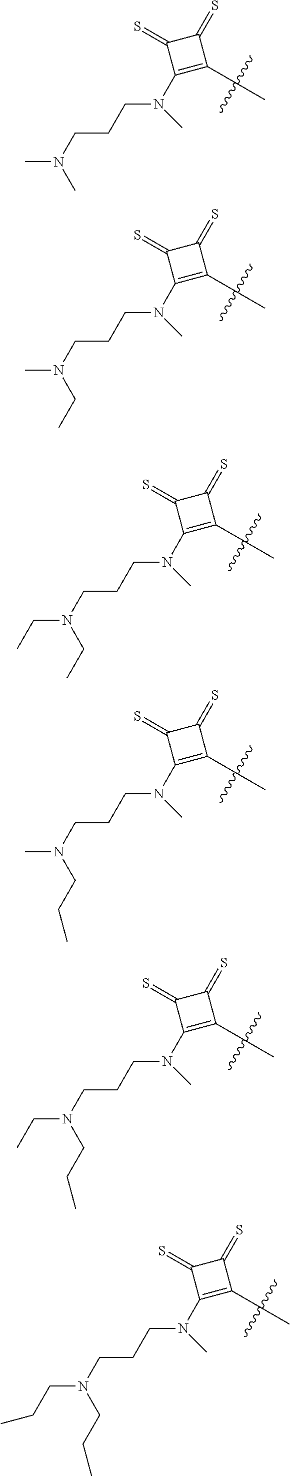 [SMILES: *c1c(N(C)CCCN(C)C)c(=S)c1=S ; *c1c(N(C)CCCN(C)CC)c(=S)c1=S ; *c1c(N(C)CCCN(CC)CC)c(=S)c1=S ; *c1c(N(C)CCCN(C)CCC)c(=S)c1=S ; *c1c(N(C)CCCN(CC)CCC)c(=S)c1=S ; *c1c(N(C)CCCN(CCC)CCC)c(=S)c1=S]

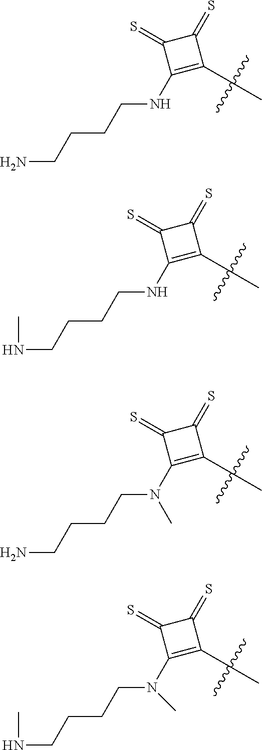 [SMILES: *c1c(NCCCCN)c(=S)c1=S ; *c1c(NCCCCNC)c(=S)c1=S ; *c1c(N(C)CCCCN)c(=S)c1=S ; *c1c(N(C)CCCCNC)c(=S)c1=S]

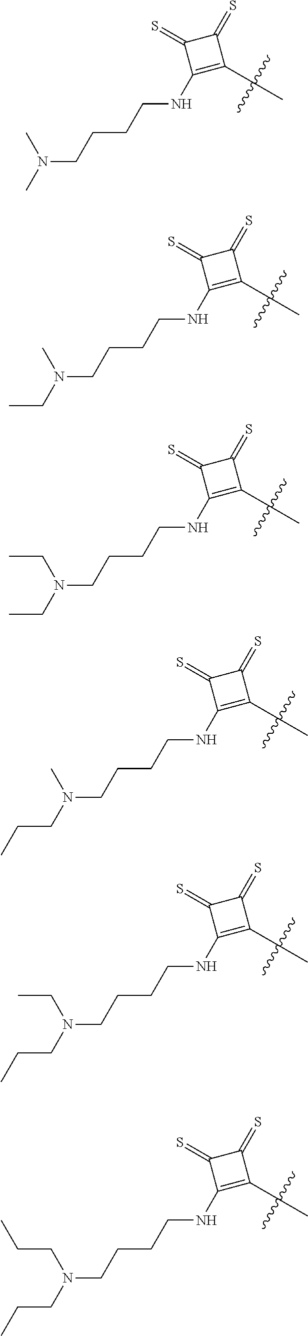 [SMILES: *c1c(NCCCCN(C)C)c(=S)c1=S ; *c1c(NCCCCN(C)CC)c(=S)c1=S ; *c1c(NCCCCN(CC)CC)c(=S)c1=S ; *c1c(NCCCCN(C)CCC)c(=S)c1=S ; *c1c(NCCCCN(CC)CCC)c(=S)c1=S ; *c1c(NCCCCN(CCC)CCC)c(=S)c1=S]

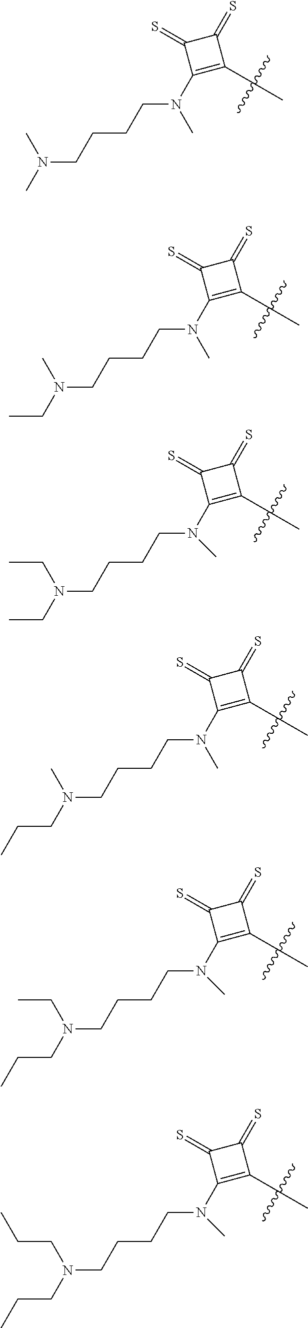 [SMILES: *c1c(N(C)CCCCN(C)C)c(=S)c1=S ; *c1c(N(C)CCCCN(C)CC)c(=S)c1=S ; *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S ; *c1c(N(C)CCCCN(CCC)CCC)c(=S)c1=S]

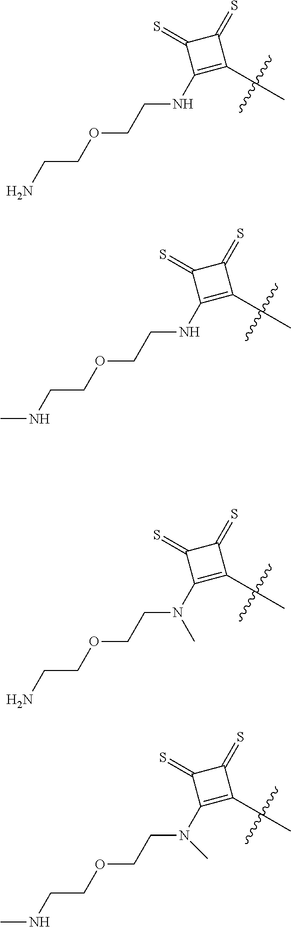 [SMILES: *c1c(NCCOCCN)c(=S)c1=S ; *c1c(NCCOCCNC)c(=S)c1=S ; *c1c(N(C)CCOCCN)c(=S)c1=S ; *c1c(N(C)CCOCCNC)c(=S)c1=S]

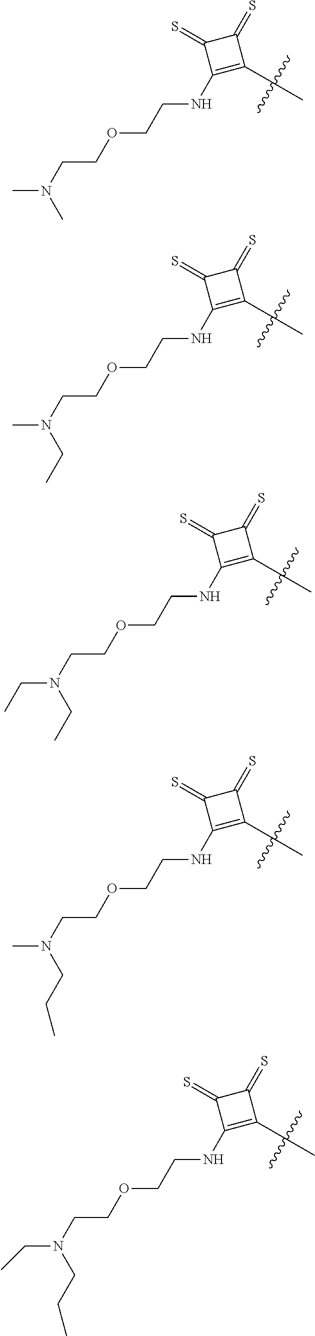 [SMILES: *c1c(NCCOCCN(C)C)c(=S)c1=S ; *c1c(NCCOCCN(C)CC)c(=S)c1=S ; *c1c(NCCOCCN(CC)CC)c(=S)c1=S ; *c1c(NCCOCCN(C)CCC)c(=S)c1=S ; *c1c(NCCOCCN(CC)CCC)c(=S)c1=S]

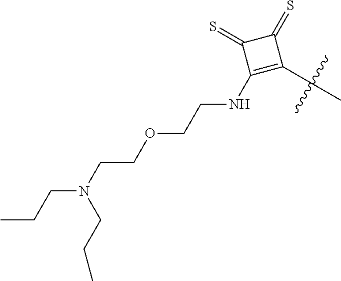 [SMILES: *c1c(NCCOCCN(CCC)CCC)c(=S)c1=S]

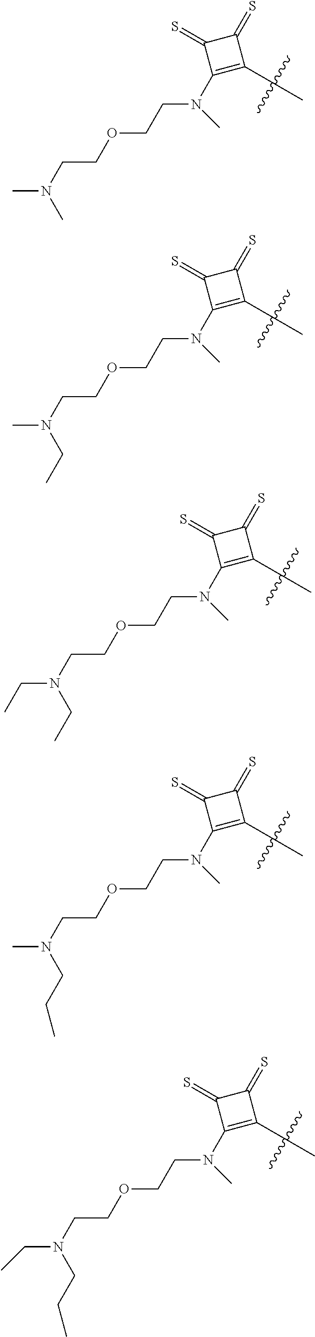 [SMILES: *c1c(N(C)CCOCCN(C)C)c(=S)c1=S ; *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S]

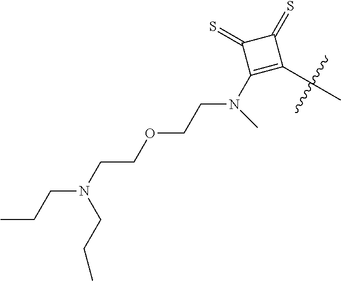 [SMILES: *c1c(N(C)CCOCCN(CCC)CCC)c(=S)c1=S]

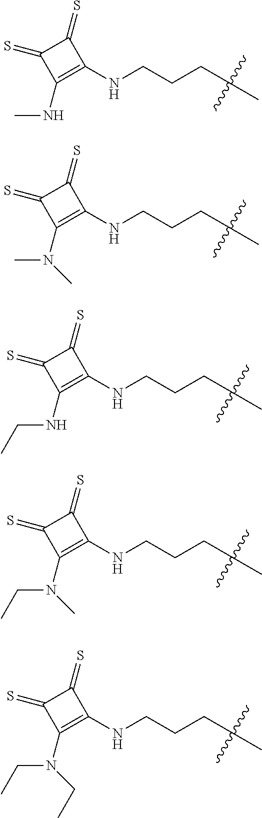

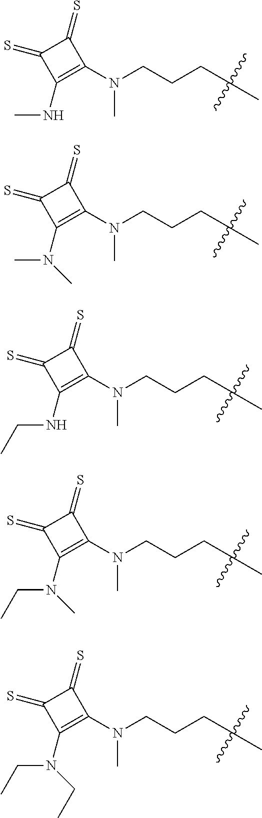

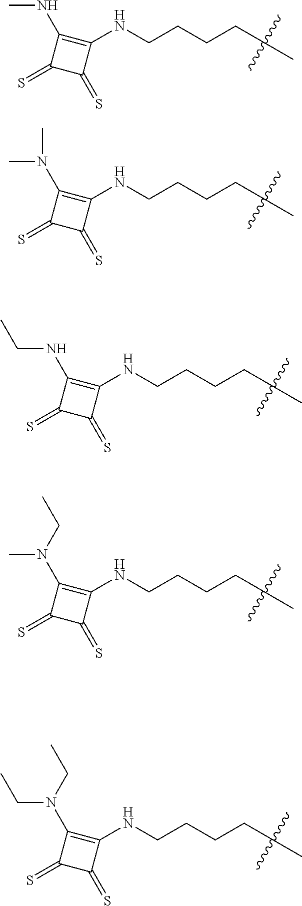 [SMILES: *CCCCNc1c(NC)c(=S)c1=S ; *CCCCNc1c(N(C)C)c(=S)c1=S ; *CCCCNc1c(NCC)c(=S)c1=S ; *CCCCNc1c(N(C)CC)c(=S)c1=S ; *CCCCNc1c(N(CC)CC)c(=S)c1=S]

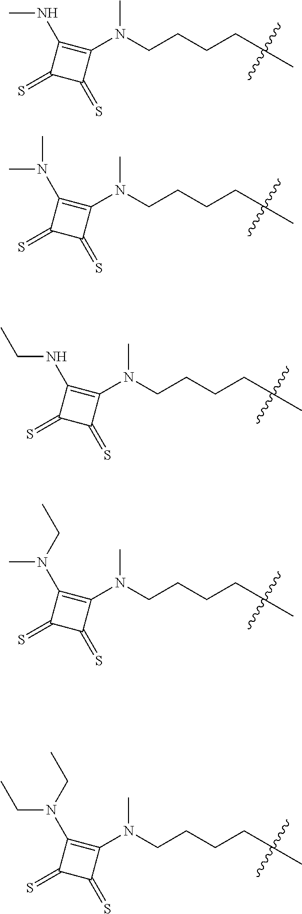 [SMILES: *CCCCN(C)c1c(NC)c(=S)c1=S ; *CCCCN(C)c1c(N(C)C)c(=S)c1=S ; *CCCCN(C)c1c(NCC)c(=S)c1=S ; *CCCCN(C)c1c(N(C)CC)c(=S)c1=S ; *CCCCN(C)c1c(N(CC)CC)c(=S)c1=S]

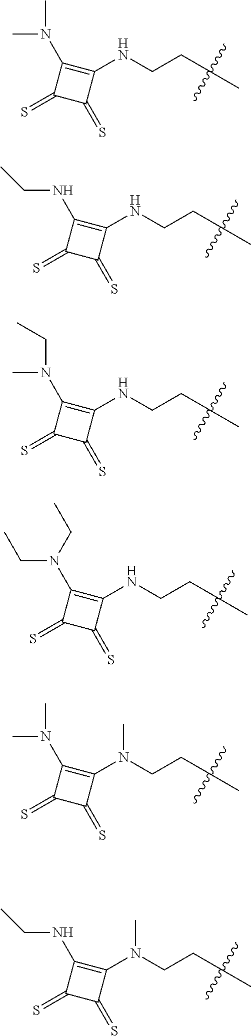 [SMILES: *CCNc1c(N(C)C)c(=S)c1=S ; *CCNc1c(NCC)c(=S)c1=S ; *CCNc1c(N(C)CC)c(=S)c1=S ; *CCNc1c(N(CC)CC)c(=S)c1=S ; *CCN(C)c1c(N(C)C)c(=S)c1=S ; *CCN(C)c1c(NCC)c(=S)c1=S]

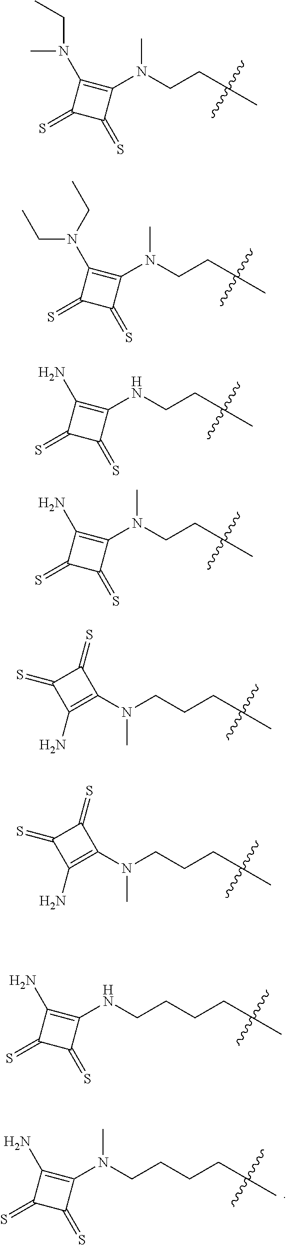

In some embodiments, R⁴ is selected from any of the following groups:

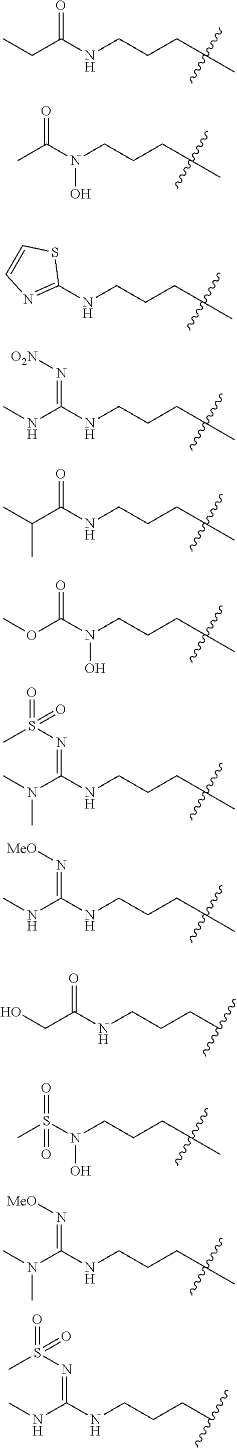

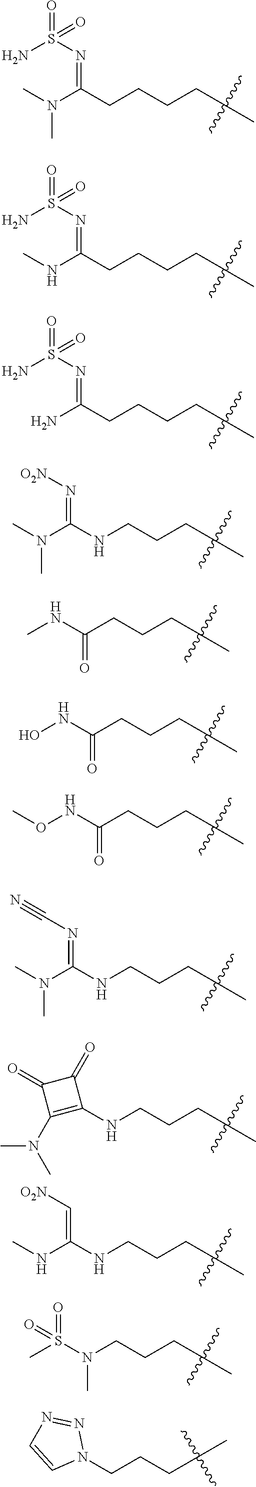

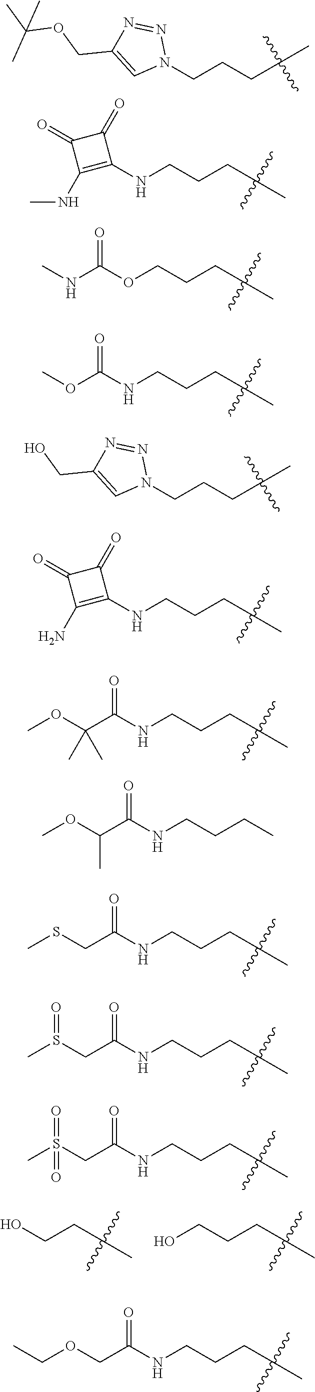

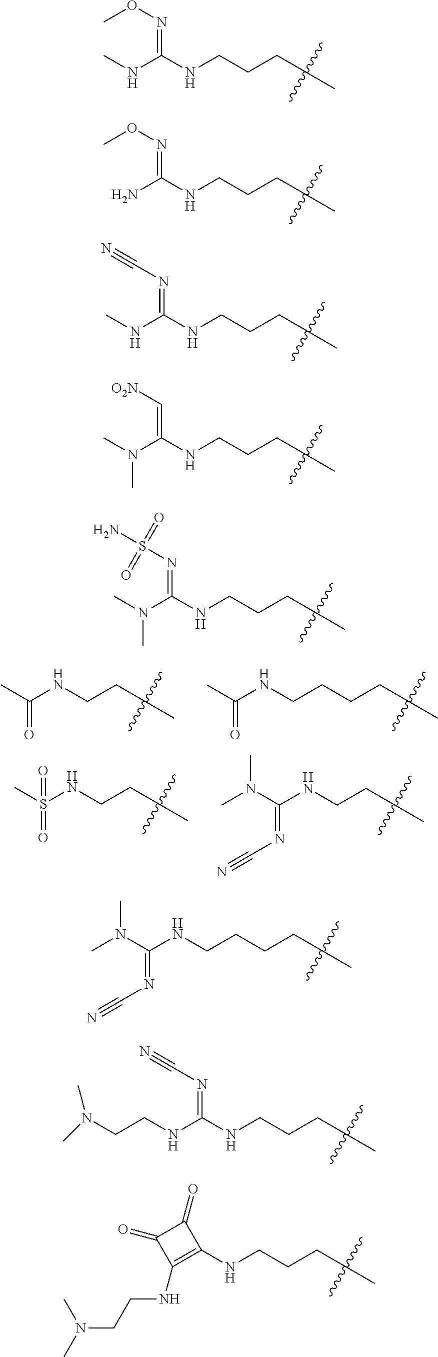

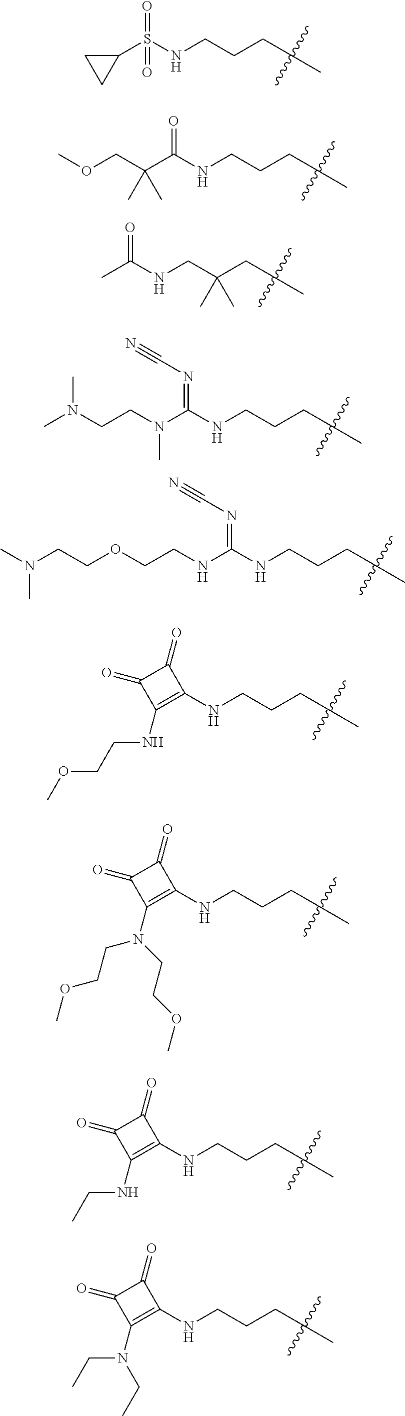

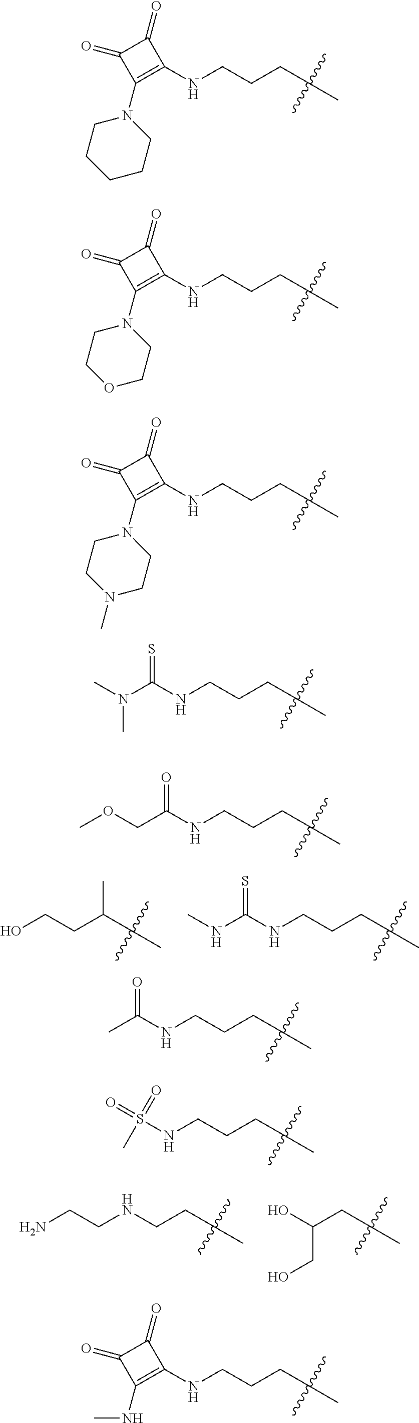

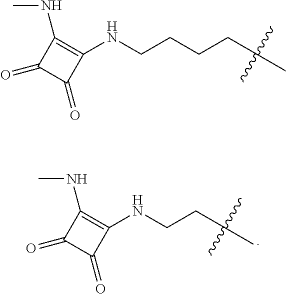 [SMILES: *CCCCNc1c(NC)c(=O)c1=O ; *CCNc1c(NC)c(=O)c1=O]

In some embodiments,

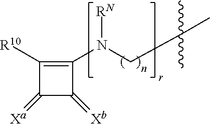

is selected from any of the following groups:

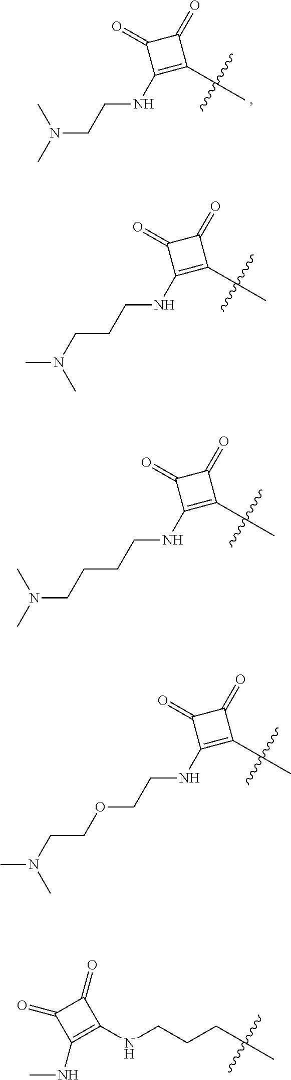 [SMILES: *c1c(NCCN(C)C)c(=O)c1=O ; *c1c(NCCCN(C)C)c(=O)c1=O ; *c1c(NCCCCN(C)C)c(=O)c1=O ; *c1c(NCCOCCN(C)C)c(=O)c1=O ; *CCCNc1c(NC)c(=O)c1=O]

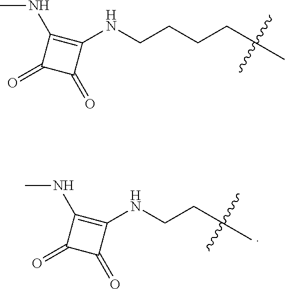 [SMILES: *CCCCNc1c(NC)c(=O)c1=O ; *CCNc1c(NC)c(=O)c1=O]

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments R^{N} is C₁ alkyl. For example, in some embodiments R^{N} is C₂ alkyl. For example, in some embodiments R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is —NH(CH₂)ₒN(R)₂, o is 2, 3, or 4.

In some embodiments wherein —NH(CH(CH₂)ₚ₁O(CH₂)_{q1}N(R)₂, p¹ is 2. In some embodiments wherein —NH(CH₂)ₚ₁O(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is —NH(CH₂)ₒN(R)₂, —NH(CH₂)ₚO(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is —NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methylpiperazinyl.

In some embodiments, each occurrence of R⁵ and R⁶ is H.

In some embodiments, the compound of Formula (I) is selected from the group consisting of:

| Cpd | Structure |
|---|---|
| I 1 | 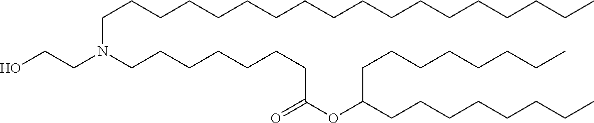 [SMILES: CCCCCCCCCCCCCCCCCCN(CCO)CCCCCCCC(=O)OC(CCCCCCCC)CCCCCCCC] |
| I 2 | 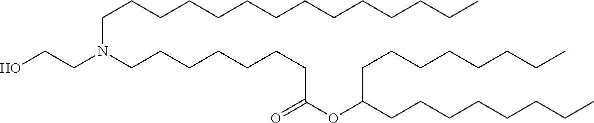 [SMILES: CCCCCCCCCCCCCCN(CCO)CCCCCCCC(=O)OC(CCCCCCCC)CCCCCCCC] |
| I 3 | 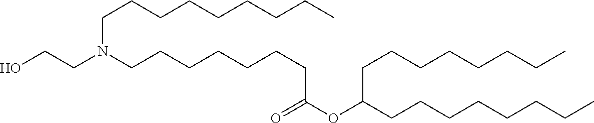 [SMILES: CCCCCCCCCN(CCO)CCCCCCCC(=O)OC(CCCCCCCC)CCCCCCCC] |
| I 4 | 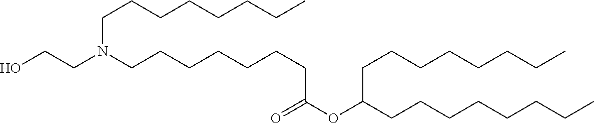 [SMILES: CCCCCCCCC(CCCCCCCC)OC(=O)CCCCCCCN(CCO)CCCCCCCC] |
| I 5 | 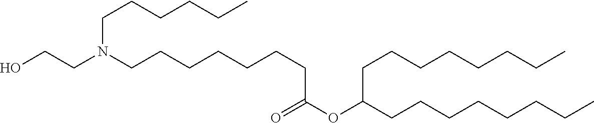 [SMILES: CCCCCCCCC(CCCCCCCC)OC(=O)CCCCCCCN(CCO)CCCCCC] |
| I 6 | 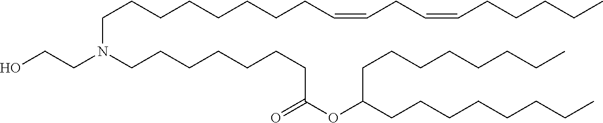 [SMILES: CCCCC/C=C\C/C=C\CCCCCCCCN(CCO)CCCCCCCC(=O)OC(CCCCCCCC)CCCCCCCC] |
| I 7 | 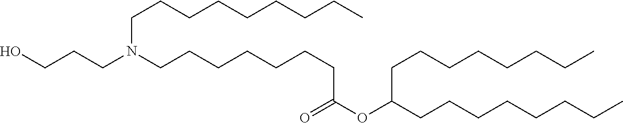 [SMILES: CCCCCCCCCN(CCCO)CCCCCCCC(=O)OC(CCCCCCCC)CCCCCCCC] |
| I 8 | 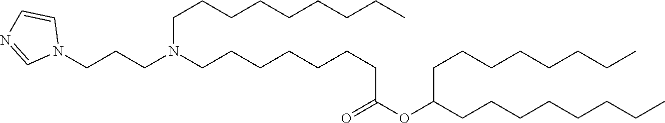 [SMILES: CCCCCCCCCN(CCCCCCCC(=O)OC(CCCCCCCC)CCCCCCCC)CCCn1ccnc1] |
| I 9 | 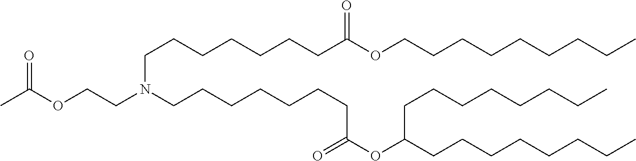 [SMILES: CCCCCCCCCOC(=O)CCCCCCCN(CCCCCCCC(=O)OC(CCCCCCCC)CCCCCCCC)CCOC(C)=O] |
| I 10 | 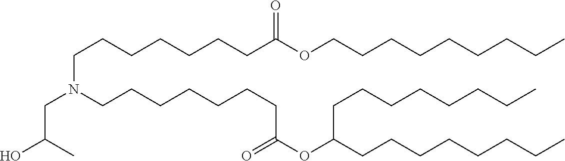 [SMILES: CCCCCCCCCOC(=O)CCCCCCCN(CCCCCCCC(=O)OC(CCCCCCCC)CCCCCCCC)CC(C)O] |
| I 11 | 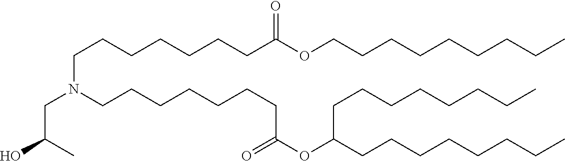 [SMILES: CCCCCCCCCOC(=O)CCCCCCCN(CCCCCCCC(=O)OC(CCCCCCCC)CCCCCCCC)C[C@@H](C)O] |
| I 12 | 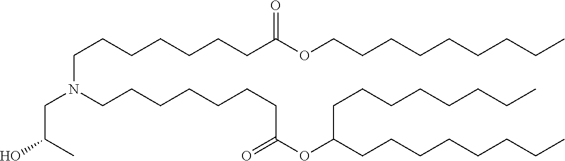 [SMILES: CCCCCCCCCOC(=O)CCCCCCCN(CCCCCCCC(=O)OC(CCCCCCCC)CCCCCCCC)C[C@H](C)O] |
| I 13 | 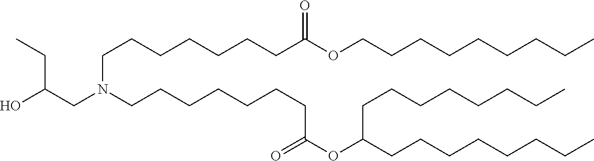 [SMILES: CCCCCCCCCOC(=O)CCCCCCCN(CCCCCCCC(=O)OC(CCCCCCCC)CCCCCCCC)CC(O)CC] |
| I 14 | 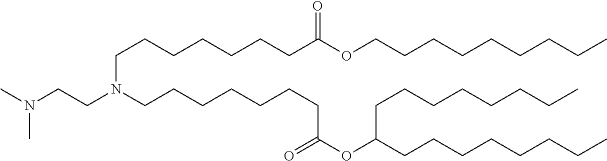 [SMILES: CCCCCCCCCOC(=O)CCCCCCCN(CCCCCCCC(=O)OC(CCCCCCCC)CCCCCCCC)CCN(C)C] |
| I 15 | 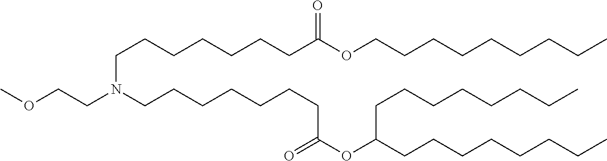 [SMILES: CCCCCCCCCOC(=O)CCCCCCCN(CCCCCCCC(=O)OC(CCCCCCCC)CCCCCCCC)CCOC] |
| I 16 | 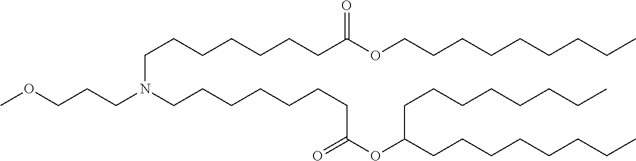 [SMILES: CCCCCCCCCOC(=O)CCCCCCCN(CCCCCCCC(=O)OC(CCCCCCCC)CCCCCCCC)CCCOC] |
| I 17 | 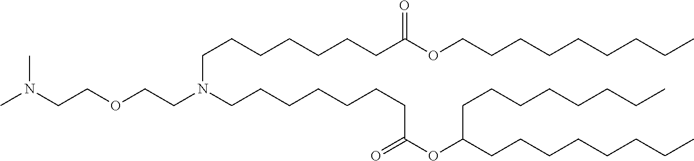 [SMILES: CCCCCCCCCOC(=O)CCCCCCCN(CCCCCCCC(=O)OC(CCCCCCCC)CCCCCCCC)CCOCCN(C)C] |
| I 18 | 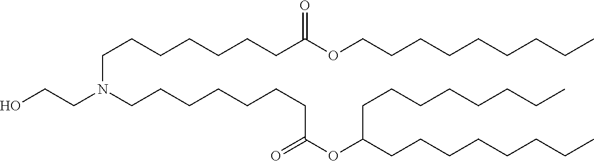 [SMILES: CCCCCCCCCOC(=O)CCCCCCCN(CCO)CCCCCCCC(=O)OC(CCCCCCCC)CCCCCCCC] |
| I 19 | 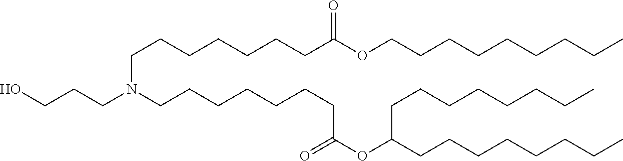 [SMILES: CCCCCCCCCOC(=O)CCCCCCCN(CCCO)CCCCCCCC(=O)OC(CCCCCCCC)CCCCCCCC] |
| I 20 | 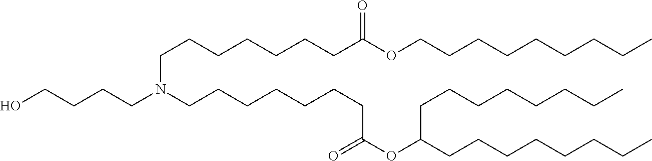 [SMILES: CCCCCCCCCOC(=O)CCCCCCCN(CCCCO)CCCCCCCC(=O)OC(CCCCCCCC)CCCCCCCC] |
| I 21 | 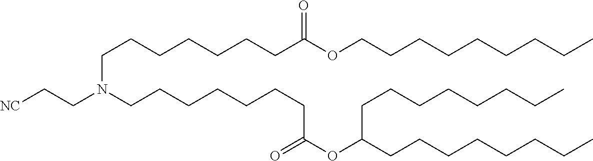 [SMILES: CCCCCCCCCOC(=O)CCCCCCCN(CCC#N)CCCCCCCC(=O)OC(CCCCCCCC)CCCCCCCC] |
| I 22 | 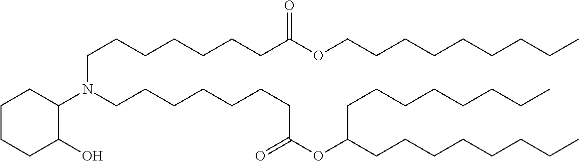 [SMILES: CCCCCCCCCOC(=O)CCCCCCCN(CCCCCCCC(=O)OC(CCCCCCCC)CCCCCCCC)C1CCCCC1O] |
| I 23 | 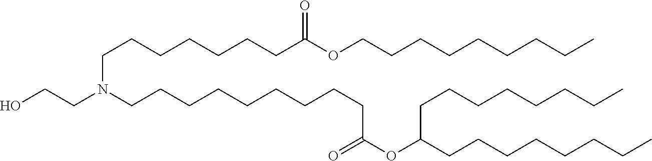 [SMILES: CCCCCCCCCOC(=O)CCCCCCCN(CCO)CCCCCCCCCC(=O)OC(CCCCCCCC)CCCCCCCC] |
| I 24 | 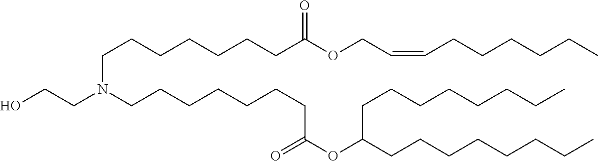 [SMILES: CCCCCC/C=C\COC(=O)CCCCCCCN(CCO)CCCCCCCC(=O)OC(CCCCCCCC)CCCCCCCC] |
| I 25 | 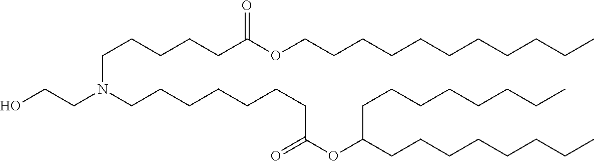 [SMILES: CCCCCCCCCCCOC(=O)CCCCCN(CCO)CCCCCCCC(=O)OC(CCCCCCCC)CCCCCCCC] |
| I 26 | 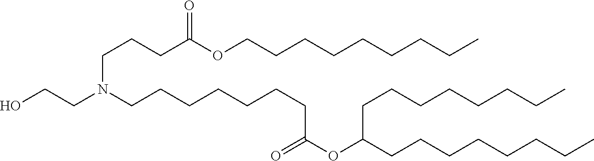 [SMILES: CCCCCCCCCOC(=O)CCCN(CCO)CCCCCCCC(=O)OC(CCCCCCCC)CCCCCCCC] |
| I 27 | 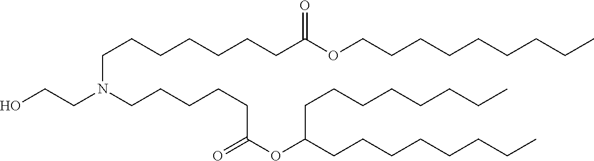 [SMILES: CCCCCCCCCOC(=O)CCCCCCCN(CCO)CCCCCC(=O)OC(CCCCCCCC)CCCCCCCC] |
| I 28 | 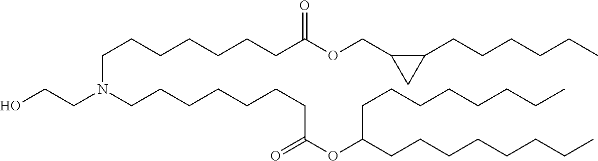 [SMILES: CCCCCCCCC(CCCCCCCC)OC(=O)CCCCCCCN(CCO)CCCCCCCC(=O)OCC1CC1CCCCCC] |
| I 29 | 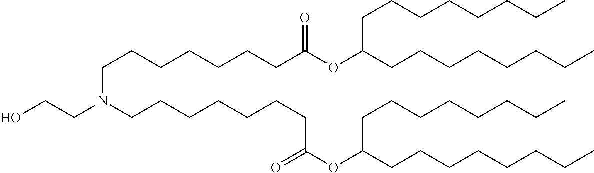 [SMILES: CCCCCCCCC(CCCCCCCC)OC(=O)CCCCCCCN(CCO)CCCCCCCC(=O)OC(CCCCCCCC)CCCCCCCC] |
| I 30 | 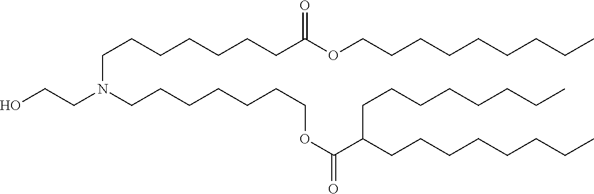 [SMILES: CCCCCCCCCOC(=O)CCCCCCCN(CCO)CCCCCCCOC(=O)C(CCCCCCCC)CCCCCCCC] |
| I 31 | 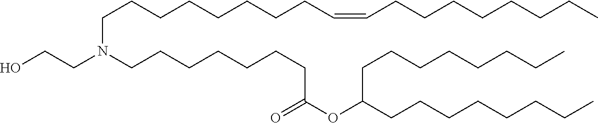 [SMILES: CCCCCCCC/C=C\CCCCCCCCN(CCO)CCCCCCCC(=O)OC(CCCCCCCC)CCCCCCCC] |
| I 32 | 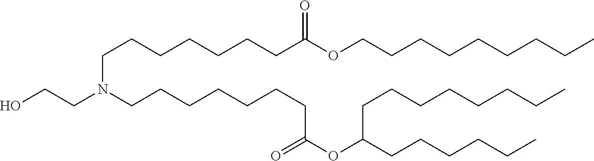 [SMILES: CCCCCCCCCOC(=O)CCCCCCCN(CCO)CCCCCCCC(=O)OC(CCCCCC)CCCCCCCC] |
| I 33 | 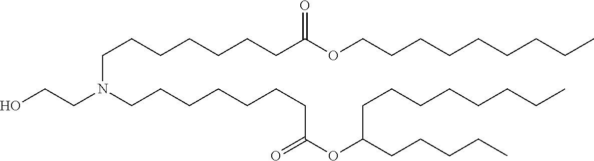 [SMILES: CCCCCCCCCOC(=O)CCCCCCCN(CCO)CCCCCCCC(=O)OC(CCCCC)CCCCCCCC] |
| I 34 | 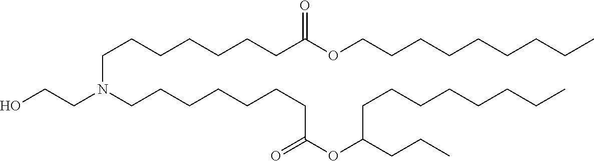 [SMILES: CCCCCCCCCOC(=O)CCCCCCCN(CCO)CCCCCCCC(=O)OC(CCC)CCCCCCCC] |
| I 35 | 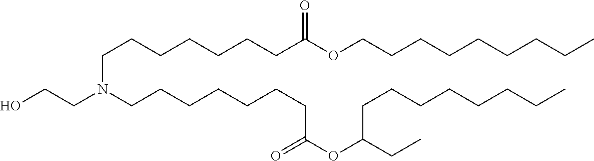 [SMILES: CCCCCCCCCOC(=O)CCCCCCCN(CCO)CCCCCCCC(=O)OC(CC)CCCCCCCC] |
| I 36 | 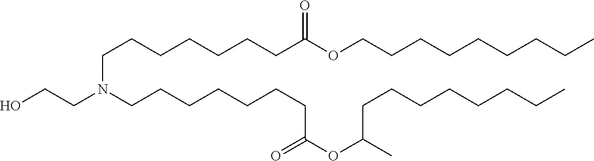 [SMILES: CCCCCCCCCOC(=O)CCCCCCCN(CCO)CCCCCCCC(=O)OC(C)CCCCCCCC] |
| I 37 | 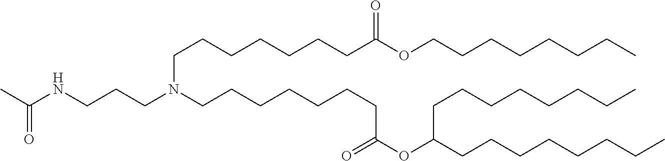 [SMILES: CCCCCCCCOC(=O)CCCCCCCN(CCCCCCCC(=O)OC(CCCCCCCC)CCCCCCCC)CCCNC(C)=O] |
| I 38 | 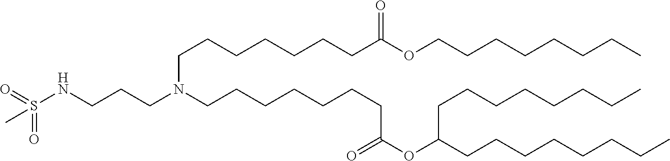 [SMILES: CCCCCCCCOC(=O)CCCCCCCN(CCCCCCCC(=O)OC(CCCCCCCC)CCCCCCCC)CCCNS(C)(=O)=O] |
| I 39 | 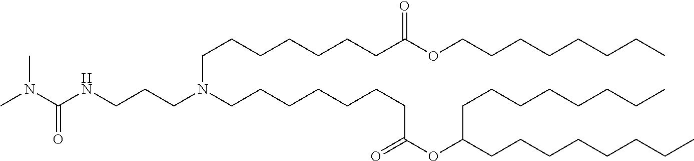 [SMILES: CCCCCCCCOC(=O)CCCCCCCN(CCCCCCCC(=O)OC(CCCCCCCC)CCCCCCCC)CCCNC(=O)N(C)C] |
| I 40 | 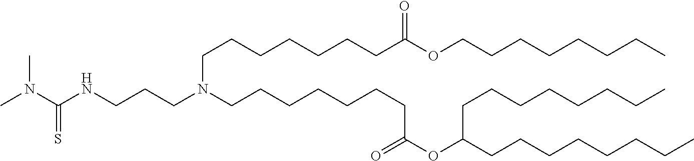 [SMILES: CCCCCCCCOC(=O)CCCCCCCN(CCCCCCCC(=O)OC(CCCCCCCC)CCCCCCCC)CCCNC(=S)N(C)C] |
| I 41 | 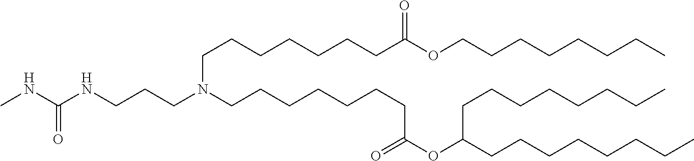 [SMILES: CCCCCCCCOC(=O)CCCCCCCN(CCCCCCCC(=O)OC(CCCCCCCC)CCCCCCCC)CCCNC(=O)NC] |
| I 42 | 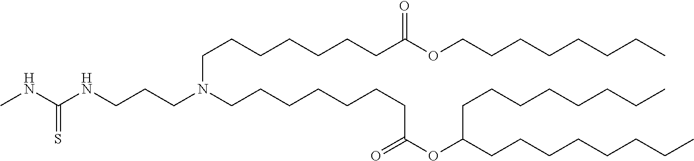 [SMILES: CCCCCCCCOC(=O)CCCCCCCN(CCCCCCCC(=O)OC(CCCCCCCC)CCCCCCCC)CCCNC(=S)NC] |
| I 43 | 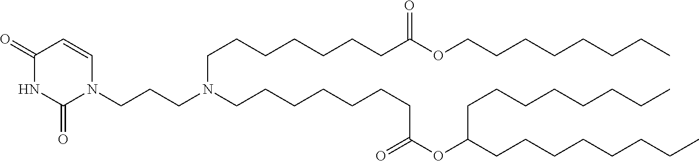 [SMILES: CCCCCCCCOC(=O)CCCCCCCN(CCCCCCCC(=O)OC(CCCCCCCC)CCCCCCCC)CCCn1ccc(=O)[nH]c1=O] |
| I 44 | 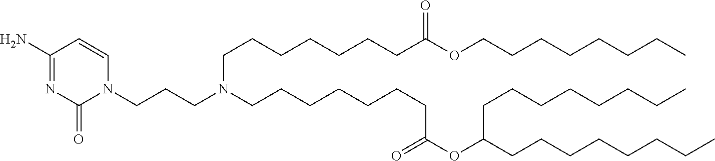 [SMILES: CCCCCCCCOC(=O)CCCCCCCN(CCCCCCCC(=O)OC(CCCCCCCC)CCCCCCCC)CCCn1ccc(N)nc1=O] |
| I 45 | 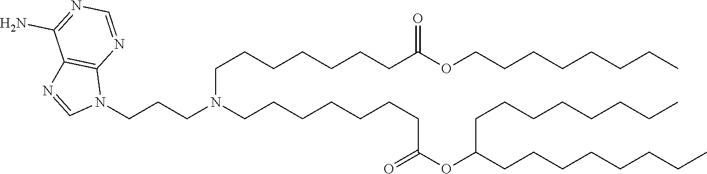 [SMILES: CCCCCCCCOC(=O)CCCCCCCN(CCCCCCCC(=O)OC(CCCCCCCC)CCCCCCCC)CCCn1cnc2c(N)ncnc21] |
| I 46 | 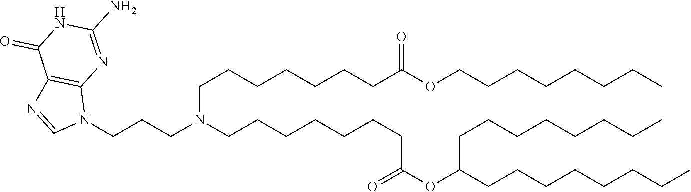 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I 47 | 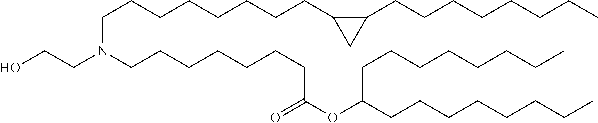 [SMILES: CCCCCCCCC(CCCCCCCC)OC(=O)CCCCCCCN(CCO)CCCCCCCCC1CC1CCCCCCCC] |
| I 48 | 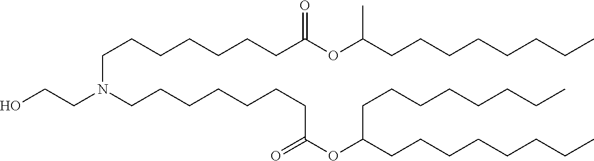 [SMILES: CCCCCCCCC(C)OC(=O)CCCCCCCN(CCO)CCCCCCCC(=O)OC(CCCCCCCC)CCCCCCCC] |
| I 49 | 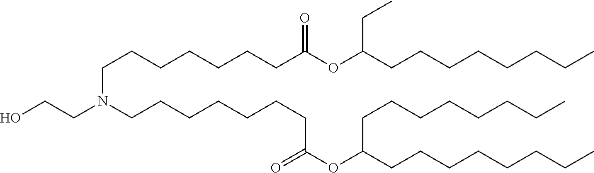 [SMILES: CCCCCCCCC(CC)OC(=O)CCCCCCCN(CCO)CCCCCCCC(=O)OC(CCCCCCCC)CCCCCCCC] |
| I 50 | 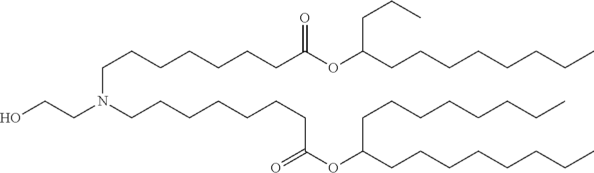 [SMILES: CCCCCCCCC(CCC)OC(=O)CCCCCCCN(CCO)CCCCCCCC(=O)OC(CCCCCCCC)CCCCCCCC] |
| I 51 | 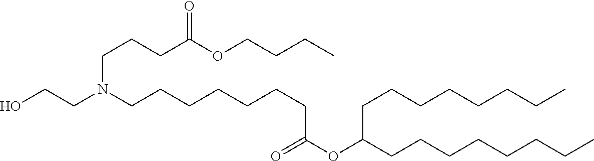 [SMILES: CCCCCCCCC(CCCCCCCC)OC(=O)CCCCCCCN(CCO)CCCC(=O)OCCCC] |
| I 52 | 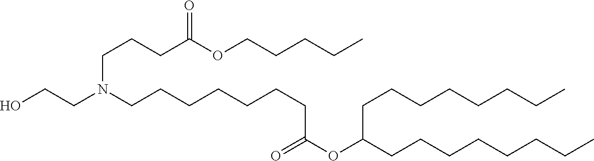 [SMILES: CCCCCCCCC(CCCCCCCC)OC(=O)CCCCCCCN(CCO)CCCC(=O)OCCCCC] |
| I 53 | 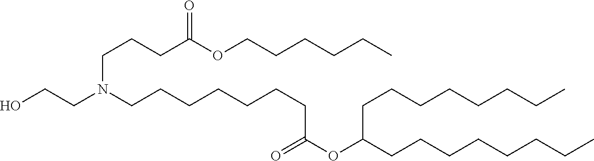 [SMILES: CCCCCCCCC(CCCCCCCC)OC(=O)CCCCCCCN(CCO)CCCC(=O)OCCCCCC] |
| I 54 | 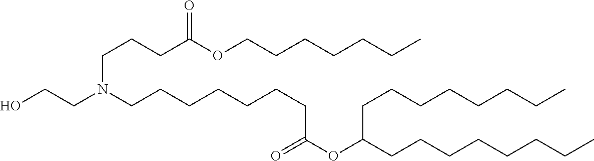 [SMILES: CCCCCCCCC(CCCCCCCC)OC(=O)CCCCCCCN(CCO)CCCC(=O)OCCCCCCC] |
| I 55 | 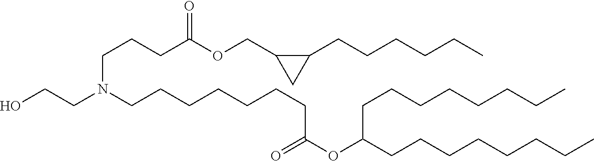 [SMILES: CCCCCCCCC(CCCCCCCC)OC(=O)CCCCCCCN(CCO)CCCC(=O)OCC1CC1CCCCCC] |
| I 56 | 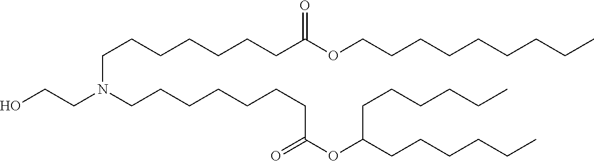 [SMILES: CCCCCCCCCOC(=O)CCCCCCCN(CCO)CCCCCCCC(=O)OC(CCCCCC)CCCCCC] |
| I 57 | 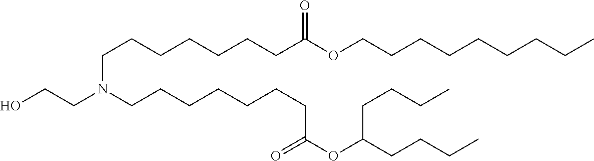 [SMILES: CCCCCCCCCOC(=O)CCCCCCCN(CCO)CCCCCCCC(=O)OC(CCCC)CCCC] |
| I 58 | 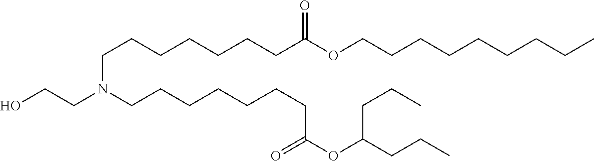 [SMILES: CCCCCCCCCOC(=O)CCCCCCCN(CCO)CCCCCCCC(=O)OC(CCC)CCC] |
| I 59 | 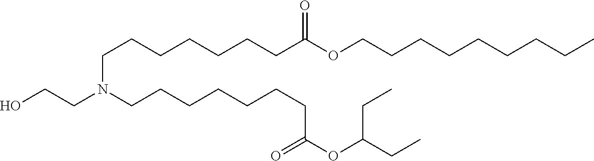 [SMILES: CCCCCCCCCOC(=O)CCCCCCCN(CCO)CCCCCCCC(=O)OC(CC)CC] |
| I 60 | 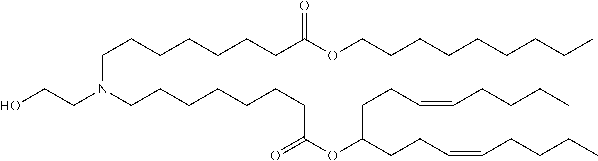 [SMILES: CCCC/C=C\CCC(CC/C=C\CCCC)OC(=O)CCCCCCCN(CCO)CCCCCCCC(=O)OCCCCCCCCC] |
| I 61 | 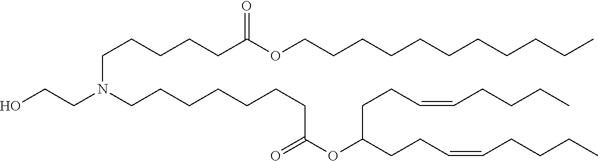 [SMILES: CCCC/C=C\CCC(CC/C=C\CCCC)OC(=O)CCCCCCCN(CCO)CCCCCC(=O)OCCCCCCCCCCC] |

In further embodiments, the compound of Formula (I I) is selected from the group consisting of:

| Cpd | Structure |
|---|---|
| I 62 | 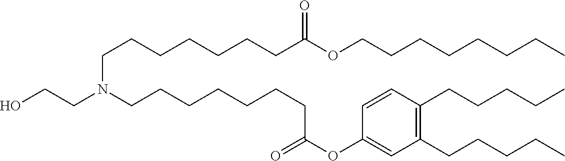 [SMILES: CCCCCCCCOC(=O)CCCCCCCN(CCO)CCCCCCCC(=O)Oc1ccc(CCCCC)c(CCCCC)c1] |
| I 63 | 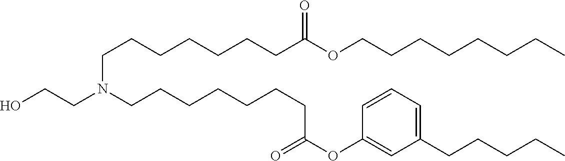 [SMILES: CCCCCCCCOC(=O)CCCCCCCN(CCO)CCCCCCCC(=O)Oc1cccc(CCCCC)c1] |
| I 64 | 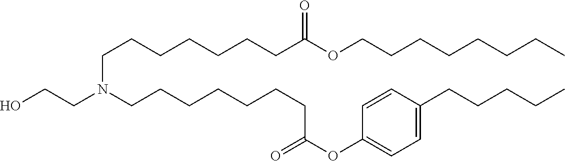 [SMILES: CCCCCCCCOC(=O)CCCCCCCN(CCO)CCCCCCCC(=O)Oc1ccc(CCCCC)cc1] |

In some embodiments, the compound of Formula (I I) or Formula (I IV) is selected from the group consisting of:

| Cpd | Structure |
|---|---|
| I 65 | 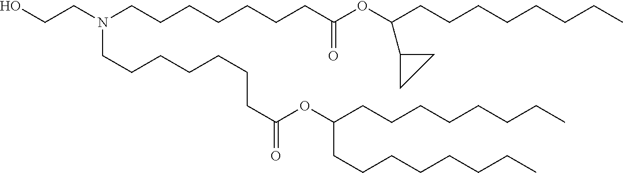 [SMILES: CCCCCCCCC(CCCCCCCC)OC(=O)CCCCCCCN(CCO)CCCCCCCC(=O)OC(CCCCCCCC)C1CC1] |
| I 66 | 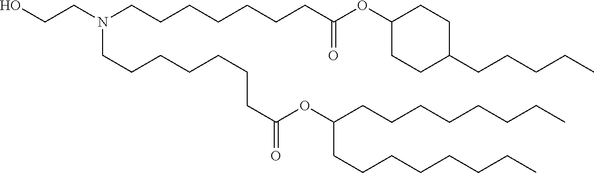 [SMILES: CCCCCCCCC(CCCCCCCC)OC(=O)CCCCCCCN(CCO)CCCCCCCC(=O)OC1CCC(CCCCC)CC1] |
| I 67 | 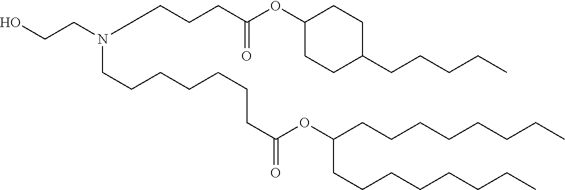 [SMILES: CCCCCCCCC(CCCCCCCC)OC(=O)CCCCCCCN(CCO)CCCC(=O)OC1CCC(CCCCC)CC1] |
| I 68 | 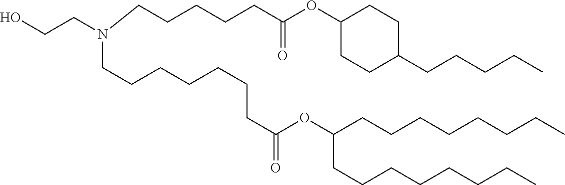 [SMILES: CCCCCCCCC(CCCCCCCC)OC(=O)CCCCCCCN(CCO)CCCCCC(=O)OC1CCC(CCCCC)CC1] |
| I 69 | 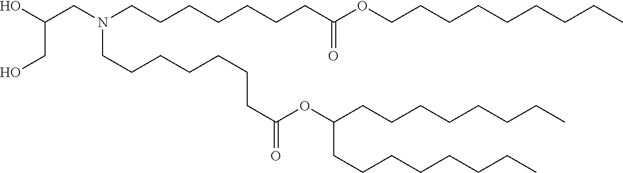 [SMILES: CCCCCCCCCOC(=O)CCCCCCCN(CCCCCCCC(=O)OC(CCCCCCCC)CCCCCCCC)CC(O)CO] |
| I 70 | 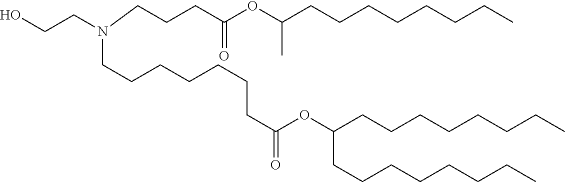 [SMILES: CCCCCCCCC(C)OC(=O)CCCN(CCO)CCCCCCCC(=O)OC(CCCCCCCC)CCCCCCCC] |
| I 71 | 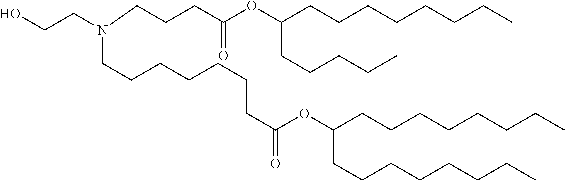 [SMILES: CCCCCCCCC(CCCCCCCC)OC(=O)CCCCCCCN(CCO)CCCC(=O)OC(CCCCC)CCCCCCCC] |
| I 72 | 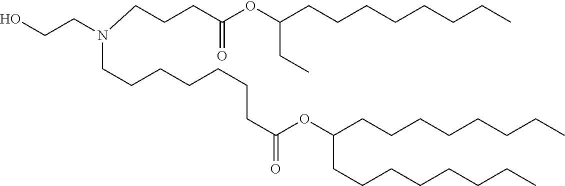 [SMILES: CCCCCCCCC(CC)OC(=O)CCCN(CCO)CCCCCCCC(=O)OC(CCCCCCCC)CCCCCCCC] |
| I 73 | 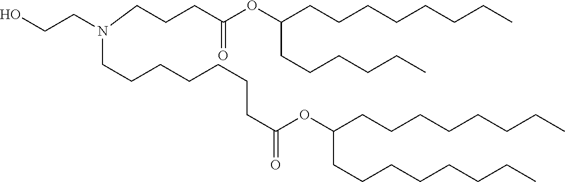 [SMILES: CCCCCCCCC(CCCCCCCC)OC(=O)CCCCCCCN(CCO)CCCC(=O)OC(CCCCCC)CCCCCCCC] |
| I 74 | 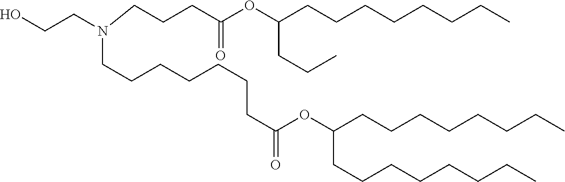 [SMILES: CCCCCCCCC(CCC)OC(=O)CCCN(CCO)CCCCCCCC(=O)OC(CCCCCCCC)CCCCCCCC] |
| I 75 | 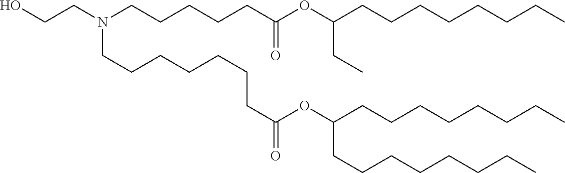 [SMILES: CCCCCCCCC(CC)OC(=O)CCCCCN(CCO)CCCCCCCC(=O)OC(CCCCCCCC)CCCCCCCC] |
| I 76 | 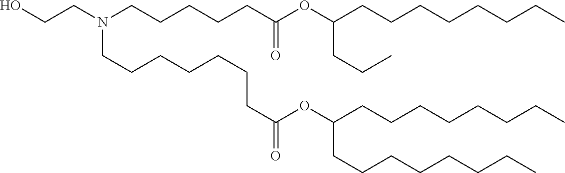 [SMILES: CCCCCCCCC(CCC)OC(=O)CCCCCN(CCO)CCCCCCCC(=O)OC(CCCCCCCC)CCCCCCCC] |
| I 77 | 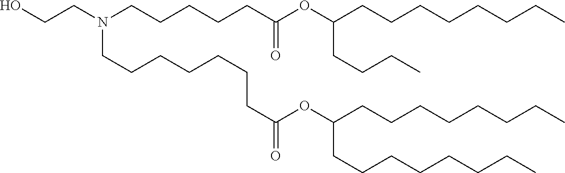 [SMILES: CCCCCCCCC(CCCC)OC(=O)CCCCCN(CCO)CCCCCCCC(=O)OC(CCCCCCCC)CCCCCCCC] |
| I 78 | 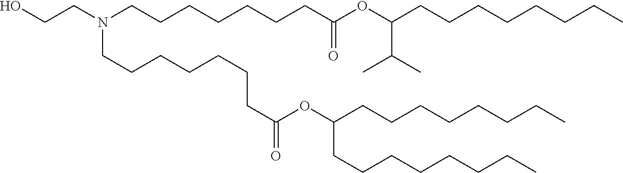 [SMILES: CCCCCCCCC(CCCCCCCC)OC(=O)CCCCCCCN(CCO)CCCCCCCC(=O)OC(CCCCCCCC)C(C)C] |
| I 79 | 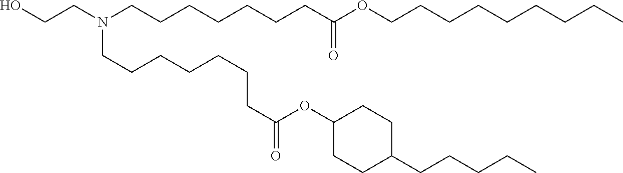 [SMILES: CCCCCCCCCOC(=O)CCCCCCCN(CCO)CCCCCCCC(=O)OC1CCC(CCCCC)CC1] |
| I 80 | 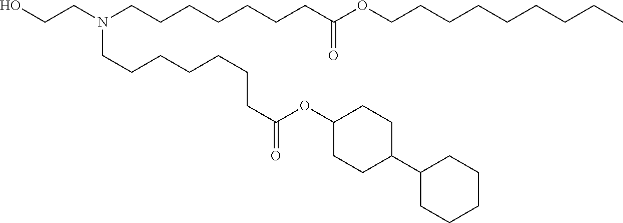 [SMILES: CCCCCCCCCOC(=O)CCCCCCCN(CCO)CCCCCCCC(=O)OC1CCC(C2CCCCC2)CC1] |
| I 81 | 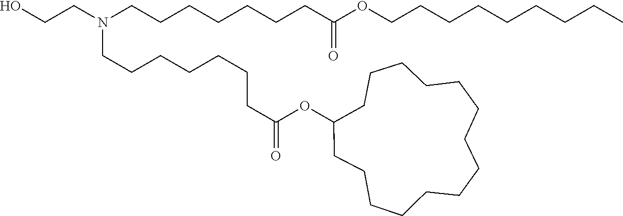 [SMILES: CCCCCCCCCOC(=O)CCCCCCCN(CCO)CCCCCCCC(=O)OC1CCCCCCCCCCCCCC1] |
| I 82 | 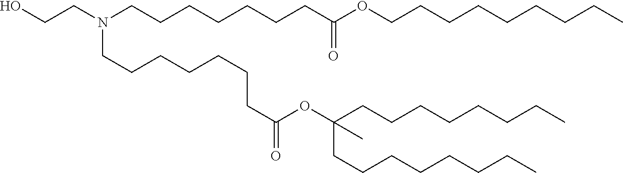 [SMILES: CCCCCCCCCOC(=O)CCCCCCCN(CCO)CCCCCCCC(=O)OC(C)(CCCCCCCC)CCCCCCCC] |
| I 83 | 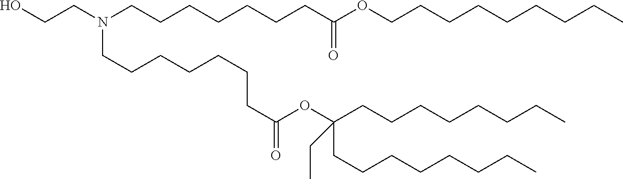 [SMILES: CCCCCCCCCOC(=O)CCCCCCCN(CCO)CCCCCCCC(=O)OC(CC)(CCCCCCCC)CCCCCCCC] |
| I 84 | 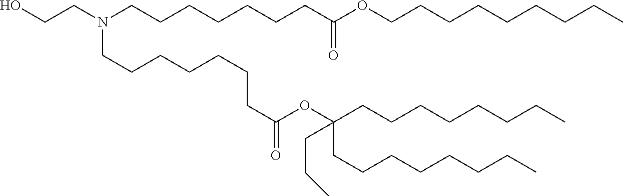 [SMILES: CCCCCCCCCOC(=O)CCCCCCCN(CCO)CCCCCCCC(=O)OC(CCC)(CCCCCCCC)CCCCCCCC] |
| I 85 | 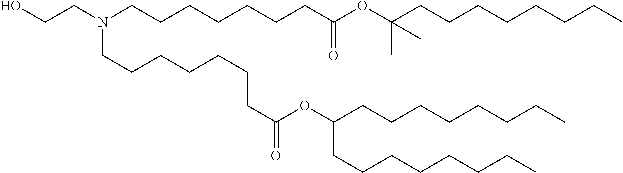 [SMILES: CCCCCCCCC(CCCCCCCC)OC(=O)CCCCCCCN(CCO)CCCCCCCC(=O)OC(C)(C)CCCCCCCC] |
| I 86 | 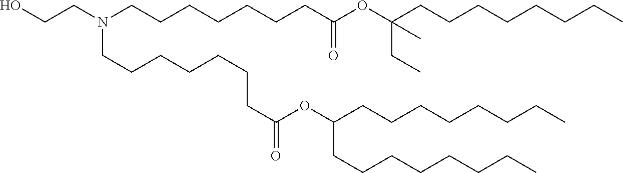 [SMILES: CCCCCCCCC(CCCCCCCC)OC(=O)CCCCCCCN(CCO)CCCCCCCC(=O)OC(C)(CC)CCCCCCCC] |
| I 87 | 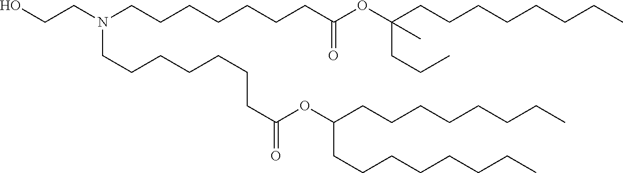 [SMILES: CCCCCCCCC(CCCCCCCC)OC(=O)CCCCCCCN(CCO)CCCCCCCC(=O)OC(C)(CCC)CCCCCCCC] |
| I 88 | 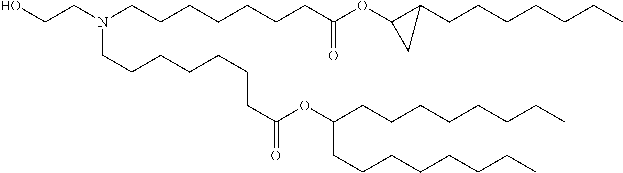 [SMILES: CCCCCCCCC(CCCCCCCC)OC(=O)CCCCCCCN(CCO)CCCCCCCC(=O)OC1CC1CCCCCCC] |
| I 89 | 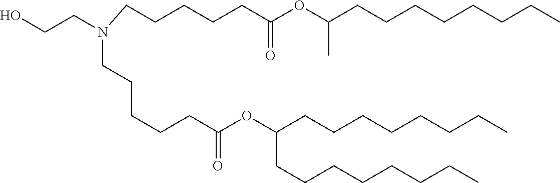 [SMILES: CCCCCCCCC(C)OC(=O)CCCCCN(CCO)CCCCCC(=O)OC(CCCCCCCC)CCCCCCCC] |
| I 90 | 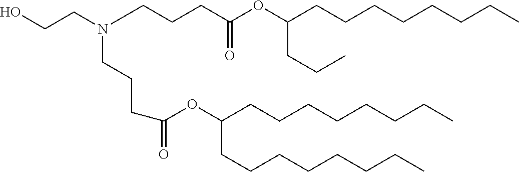 [SMILES: CCCCCCCCC(CCC)OC(=O)CCCN(CCO)CCCC(=O)OC(CCCCCCCC)CCCCCCCC] |
| I 91 | 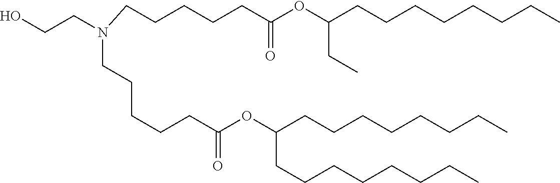 [SMILES: CCCCCCCCC(CC)OC(=O)CCCCCN(CCO)CCCCCC(=O)OC(CCCCCCCC)CCCCCCCC] |
| I 92 | 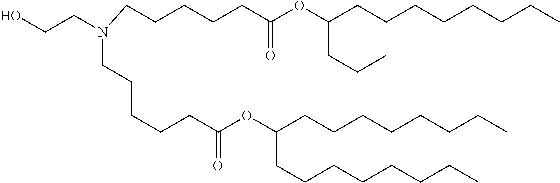 [SMILES: CCCCCCCCC(CCC)OC(=O)CCCCCN(CCO)CCCCCC(=O)OC(CCCCCCCC)CCCCCCCC] |
| I 93 | 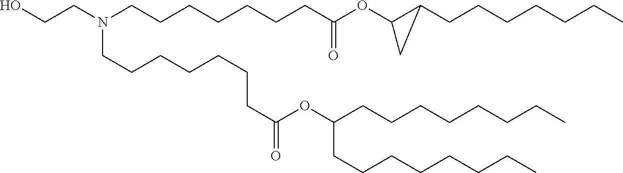 [SMILES: CCCCCCCCC(CCCCCCCC)OC(=O)CCCCCCCN(CCO)CCCCCCCC(=O)OC1CC1CCCCCCC] |
| I 94 | 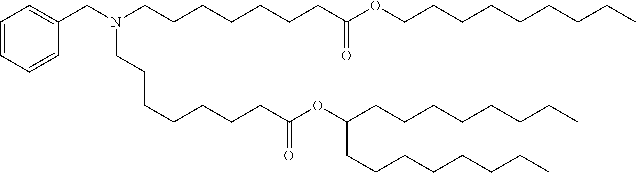 [SMILES: CCCCCCCCCOC(=O)CCCCCCCN(CCCCCCCC(=O)OC(CCCCCCCC)CCCCCCCC)Cc1ccccc1] |
| I 95 | 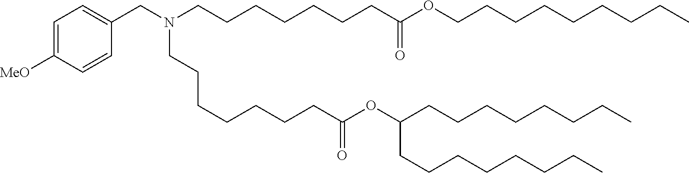 [SMILES: CCCCCCCCCOC(=O)CCCCCCCN(CCCCCCCC(=O)OC(CCCCCCCC)CCCCCCCC)Cc1ccc(OC)cc1] |
| I 96 | 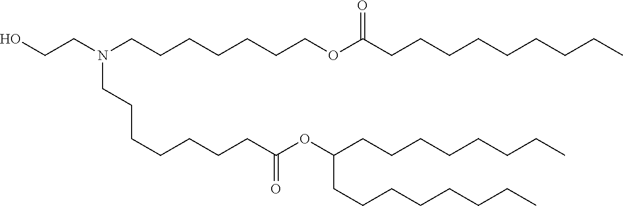 [SMILES: CCCCCCCCCC(=O)OCCCCCCCN(CCO)CCCCCCCC(=O)OC(CCCCCCCC)CCCCCCCC] |
| I 97 | 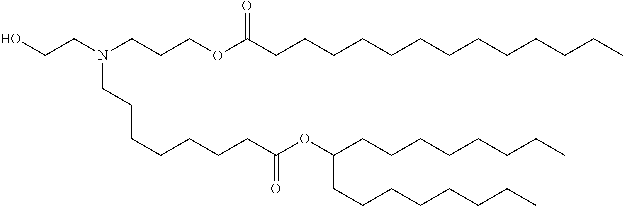 [SMILES: CCCCCCCCCCCCCC(=O)OCCCN(CCO)CCCCCCCC(=O)OC(CCCCCCCC)CCCCCCCC] |
| I 98 | 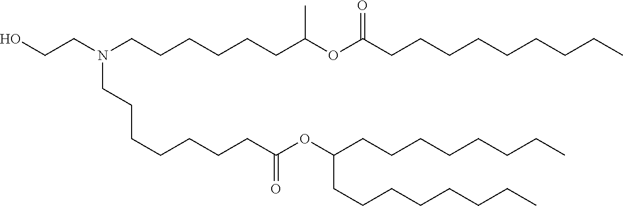 [SMILES: CCCCCCCCCC(=O)OC(C)CCCCCCN(CCO)CCCCCCCC(=O)OC(CCCCCCCC)CCCCCCCC] |
| I 99 | 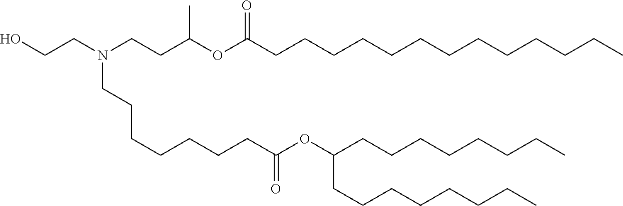 [SMILES: CCCCCCCCCCCCCC(=O)OC(C)CCN(CCO)CCCCCCCC(=O)OC(CCCCCCCC)CCCCCCCC] |
| I 100 | 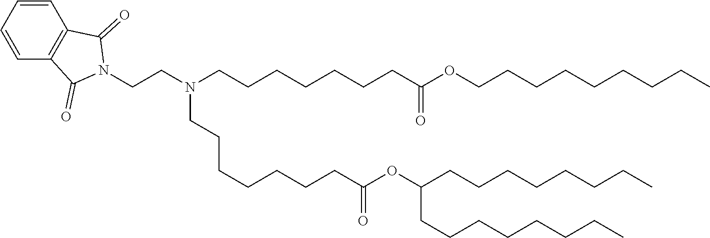 [SMILES: CCCCCCCCCOC(=O)CCCCCCCN(CCCCCCCC(=O)OC(CCCCCCCC)CCCCCCCC)CCN1C(=O)c2ccccc2C1=O] |
| I 101 | 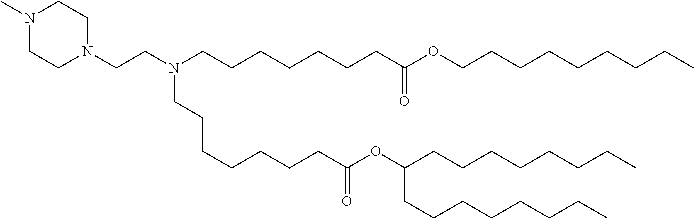 [SMILES: CCCCCCCCCOC(=O)CCCCCCCN(CCCCCCCC(=O)OC(CCCCCCCC)CCCCCCCC)CCN1CCN(C)CC1] |
| I 102 | 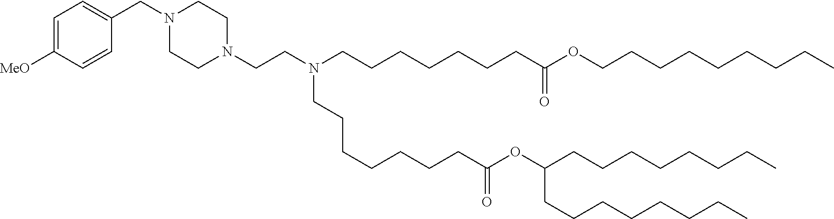 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I 103 | 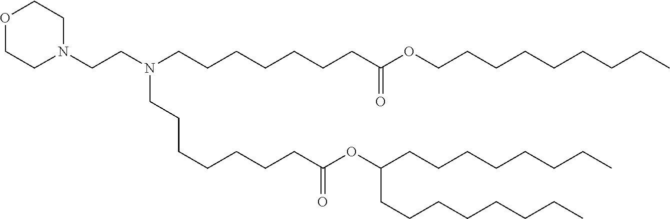 [SMILES: CCCCCCCCCOC(=O)CCCCCCCN(CCCCCCCC(=O)OC(CCCCCCCC)CCCCCCCC)CCN1CCOCC1] |
| I 104 | 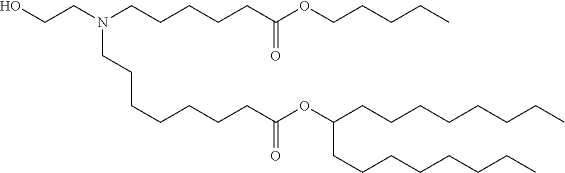 [SMILES: CCCCCCCCC(CCCCCCCC)OC(=O)CCCCCCCN(CCO)CCCCCC(=O)OCCCCC] |
| I 105 | 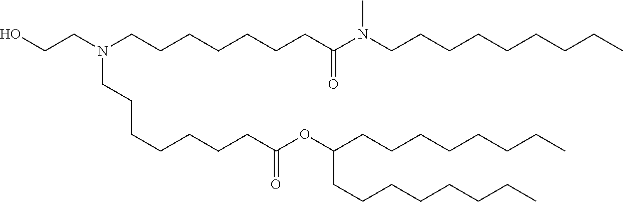 [SMILES: CCCCCCCCCN(C)C(=O)CCCCCCCN(CCO)CCCCCCCC(=O)OC(CCCCCCCC)CCCCCCCC] |
| I 106 | 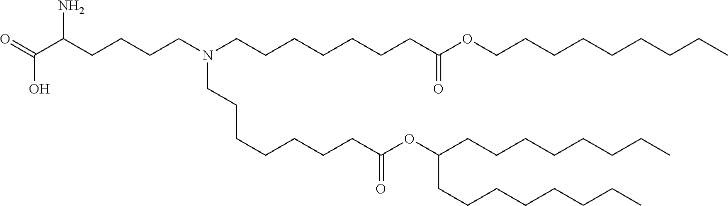 [SMILES: CCCCCCCCCOC(=O)CCCCCCCN(CCCCCCCC(=O)OC(CCCCCCCC)CCCCCCCC)CCCCC(N)C(=O)O] |
| I 107 | 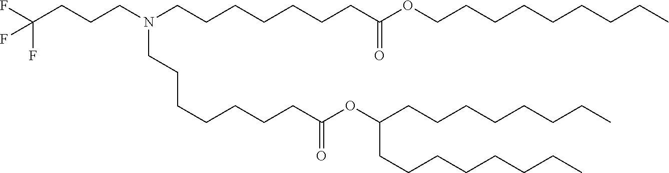 [SMILES: CCCCCCCCCOC(=O)CCCCCCCN(CCCCCCCC(=O)OC(CCCCCCCC)CCCCCCCC)CCCC(F)(F)F] |
| I 108 | 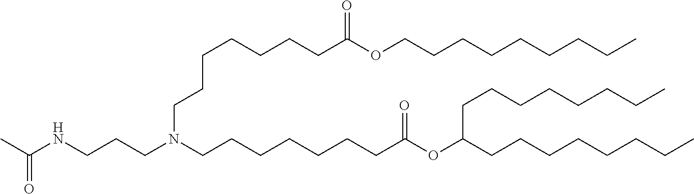 [SMILES: CCCCCCCCCOC(=O)CCCCCCCN(CCCCCCCC(=O)OC(CCCCCCCC)CCCCCCCC)CCCNC(C)=O] |
| I 109 | 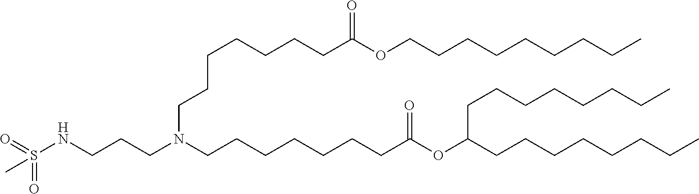 [SMILES: CCCCCCCCCOC(=O)CCCCCCCN(CCCCCCCC(=O)OC(CCCCCCCC)CCCCCCCC)CCCNS(C)(=O)=O] |
| I 110 | 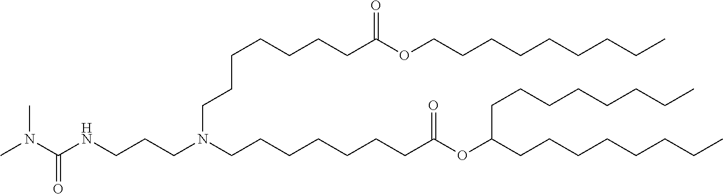 [SMILES: CCCCCCCCCOC(=O)CCCCCCCN(CCCCCCCC(=O)OC(CCCCCCCC)CCCCCCCC)CCCNC(=O)N(C)C] |
| I 111 | 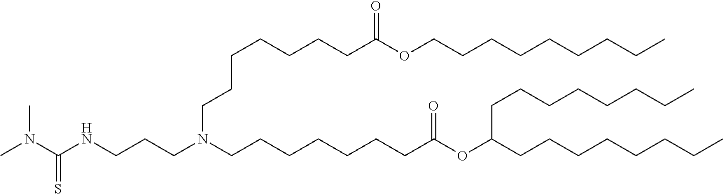 [SMILES: CCCCCCCCCOC(=O)CCCCCCCN(CCCCCCCC(=O)OC(CCCCCCCC)CCCCCCCC)CCCNC(=S)N(C)C] |
| I 112 | 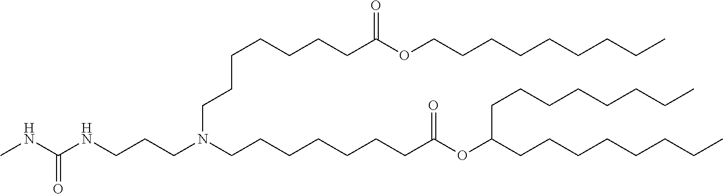 [SMILES: CCCCCCCCCOC(=O)CCCCCCCN(CCCCCCCC(=O)OC(CCCCCCCC)CCCCCCCC)CCCNC(=O)NC] |
| I 113 | 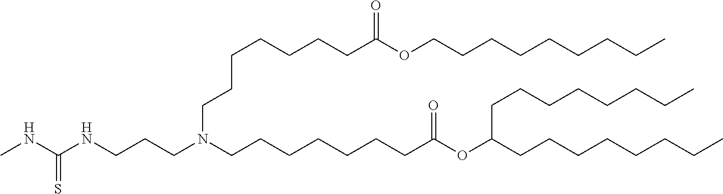 [SMILES: CCCCCCCCCOC(=O)CCCCCCCN(CCCCCCCC(=O)OC(CCCCCCCC)CCCCCCCC)CCCNC(=S)NC] |
| I 114 | 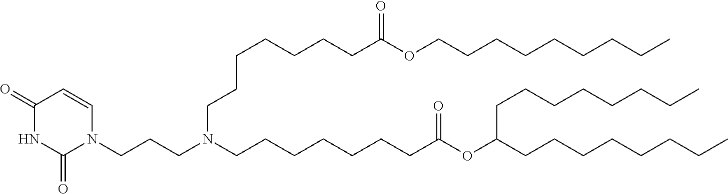 [SMILES: CCCCCCCCCOC(=O)CCCCCCCN(CCCCCCCC(=O)OC(CCCCCCCC)CCCCCCCC)CCCn1ccc(=O)[nH]c1=O] |
| I 115 | 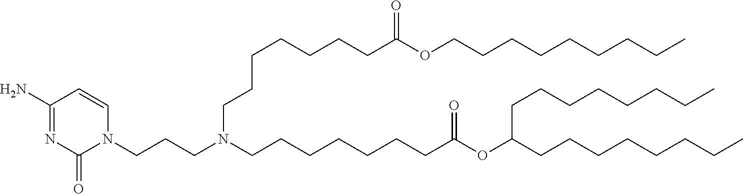 [SMILES: CCCCCCCCCOC(=O)CCCCCCCN(CCCCCCCC(=O)OC(CCCCCCCC)CCCCCCCC)CCCn1ccc(N)nc1=O] |
| I 116 | 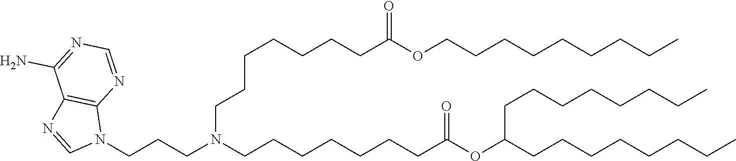 [SMILES: CCCCCCCCCOC(=O)CCCCCCCN(CCCCCCCC(=O)OC(CCCCCCCC)CCCCCCCC)CCCn1cnc2c(N)ncnc21] |
| I 117 | 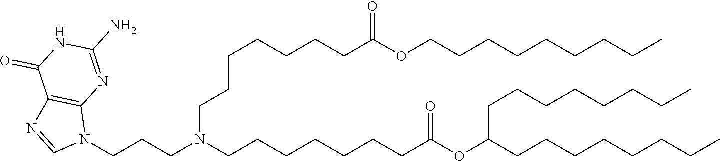 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I 118 | 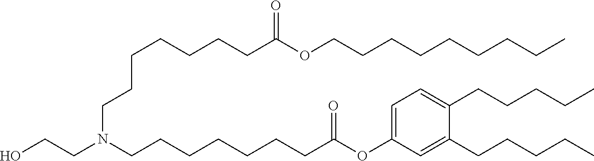 [SMILES: CCCCCCCCCOC(=O)CCCCCCCN(CCO)CCCCCCCC(=O)Oc1ccc(CCCCC)c(CCCCC)c1] |
| I 119 | 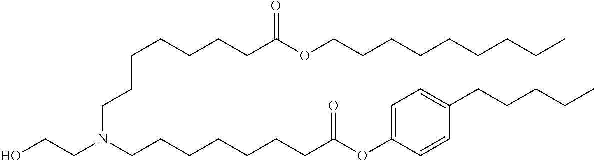 [SMILES: CCCCCCCCCOC(=O)CCCCCCCN(CCO)CCCCCCCC(=O)Oc1ccc(CCCCC)cc1] |
| I 120 | 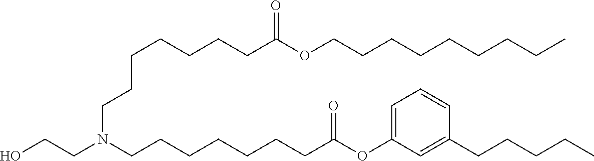 [SMILES: CCCCCCCCCOC(=O)CCCCCCCN(CCO)CCCCCCCC(=O)Oc1cccc(CCCCC)c1] |
| I 121 | 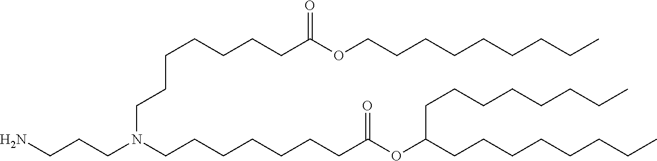 [SMILES: CCCCCCCCCOC(=O)CCCCCCCN(CCCN)CCCCCCCC(=O)OC(CCCCCCCC)CCCCCCCC] |
| I 122 | 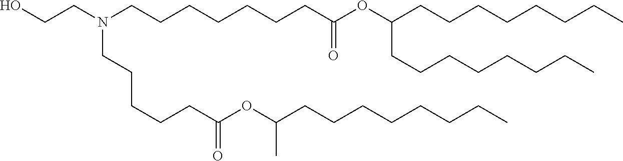 [SMILES: CCCCCCCCC(C)OC(=O)CCCCCN(CCO)CCCCCCCC(=O)OC(CCCCCCCC)CCCCCCCC] |
| I 123 | 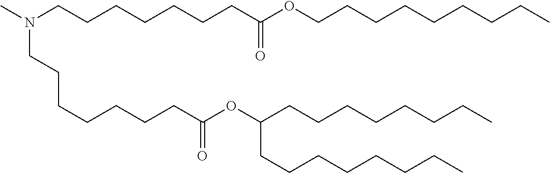 [SMILES: CCCCCCCCCOC(=O)CCCCCCCN(C)CCCCCCCC(=O)OC(CCCCCCCC)CCCCCCCC] |
| I 124 | 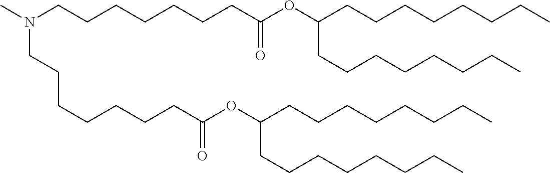 [SMILES: CCCCCCCCC(CCCCCCCC)OC(=O)CCCCCCCN(C)CCCCCCCC(=O)OC(CCCCCCCC)CCCCCCCC] |
| I 125 | 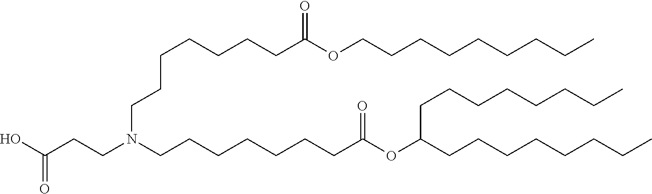 [SMILES: CCCCCCCCCOC(=O)CCCCCCCN(CCCCCCCC(=O)OC(CCCCCCCC)CCCCCCCC)CCC(=O)O] |
| I 126 | 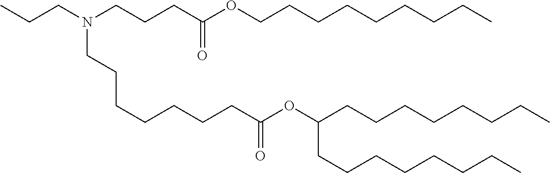 [SMILES: CCCCCCCCCOC(=O)CCCN(CCC)CCCCCCCC(=O)OC(CCCCCCCC)CCCCCCCC] |
| I 127 | 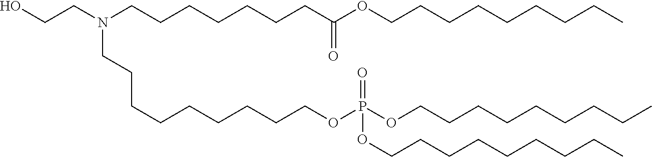 [SMILES: CCCCCCCCCOC(=O)CCCCCCCN(CCO)CCCCCCCCCOP(=O)(OCCCCCCCCC)OCCCCCCCCC] |
| I 128 | 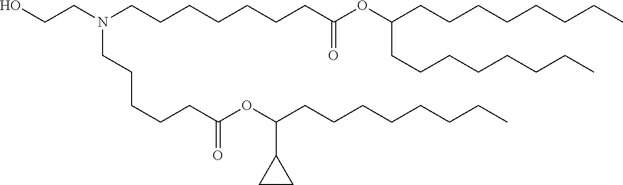 [SMILES: CCCCCCCCC(CCCCCCCC)OC(=O)CCCCCCCN(CCO)CCCCCC(=O)OC(CCCCCCCC)C1CC1] |
| I 129 | 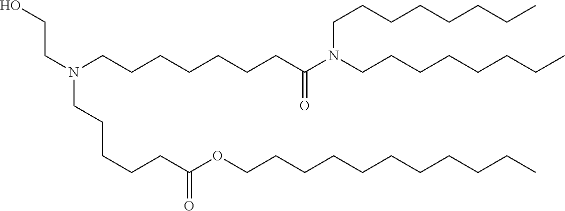 [SMILES: CCCCCCCCCCCOC(=O)CCCCCN(CCO)CCCCCCCC(=O)N(CCCCCCCC)CCCCCCCC] |
| I 130 | 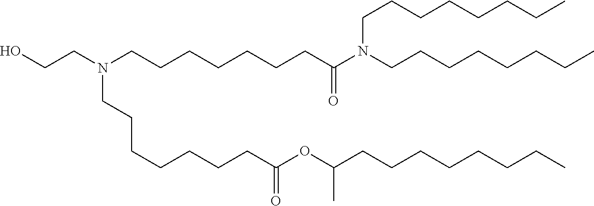 [SMILES: CCCCCCCCC(C)OC(=O)CCCCCCCN(CCO)CCCCCCCC(=O)N(CCCCCCCC)CCCCCCCC] |
| I 131 | 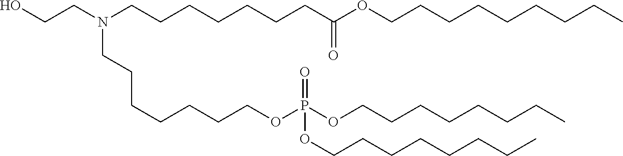 [SMILES: CCCCCCCCCOC(=O)CCCCCCCN(CCO)CCCCCCCOP(=O)(OCCCCCCCC)OCCCCCCCC] |
| I 132 | 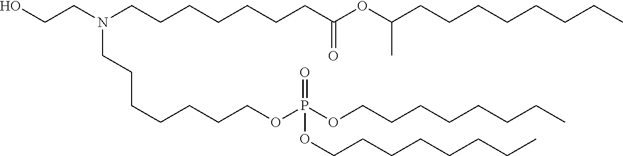 [SMILES: CCCCCCCCOP(=O)(OCCCCCCCC)OCCCCCCCN(CCO)CCCCCCCC(=O)OC(C)CCCCCCCC] |
| I 133 | 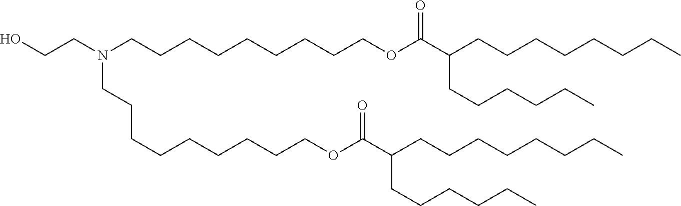 [SMILES: CCCCCCCCC(CCCCCC)C(=O)OCCCCCCCCCN(CCO)CCCCCCCCCOC(=O)C(CCCCCC)CCCCCCCC] |
| I 134 | 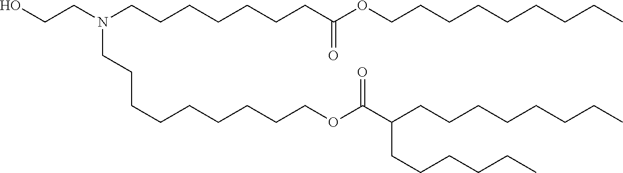 [SMILES: CCCCCCCCCOC(=O)CCCCCCCN(CCO)CCCCCCCCCOC(=O)C(CCCCCC)CCCCCCCC] |
| I 135 | 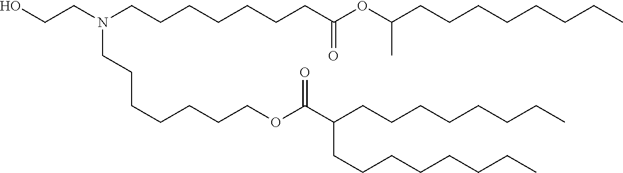 [SMILES: CCCCCCCCC(C)OC(=O)CCCCCCCN(CCO)CCCCCCCOC(=O)C(CCCCCCCC)CCCCCCCC] |
| I 136 | 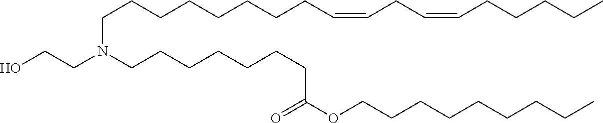 [SMILES: CCCCC/C=C\C/C=C\CCCCCCCCN(CCO)CCCCCCCC(=O)OCCCCCCCCC] |
| I 137 | 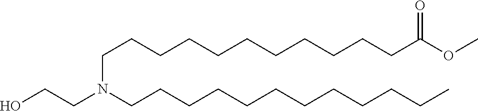 [SMILES: CCCCCCCCCCCCN(CCO)CCCCCCCCCCCC(=O)OC] |
| I 138 | 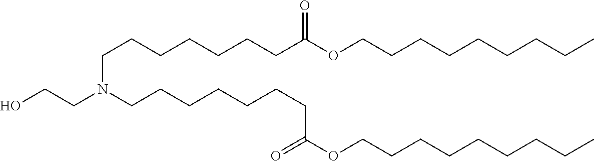 [SMILES: CCCCCCCCCOC(=O)CCCCCCCN(CCO)CCCCCCCC(=O)OCCCCCCCCC] |
| I 139 | 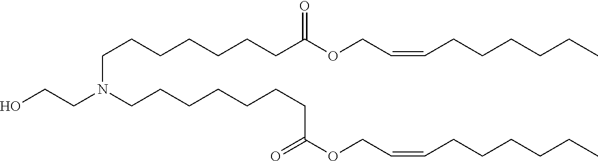 [SMILES: CCCCCC/C=C\COC(=O)CCCCCCCN(CCO)CCCCCCCC(=O)OC/C=C\CCCCCC] |
| I 140 | 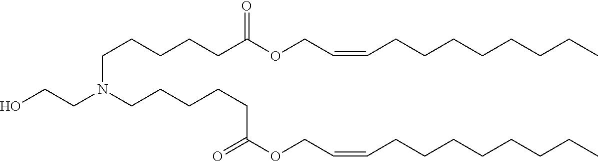 [SMILES: CCCCCCCC/C=C\COC(=O)CCCCCN(CCO)CCCCCC(=O)OC/C=C\CCCCCCCC] |
| I 141 | 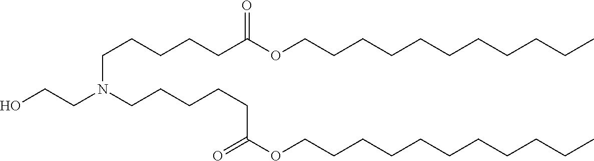 [SMILES: CCCCCCCCCCCOC(=O)CCCCCN(CCO)CCCCCC(=O)OCCCCCCCCCCC] |
| I 142 | 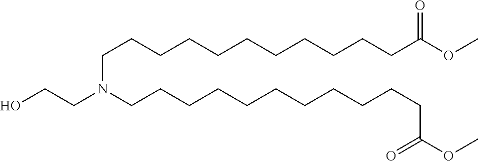 [SMILES: COC(=O)CCCCCCCCCCCN(CCO)CCCCCCCCCCCC(=O)OC] |
| I 143 | 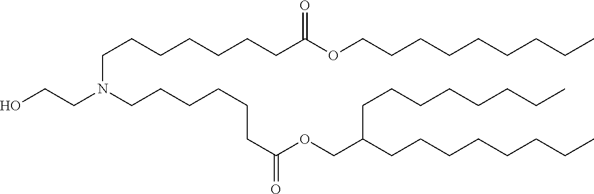 [SMILES: CCCCCCCCCOC(=O)CCCCCCCN(CCO)CCCCCCC(=O)OCC(CCCCCCCC)CCCCCCCC] |
| I 144 | 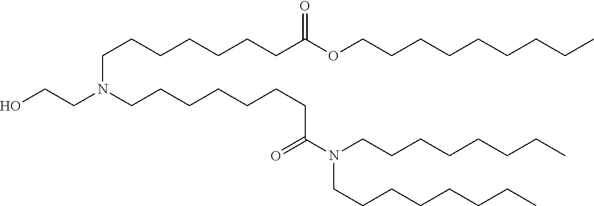 [SMILES: CCCCCCCCCOC(=O)CCCCCCCN(CCO)CCCCCCCC(=O)N(CCCCCCCC)CCCCCCCC] |
| I 145 | 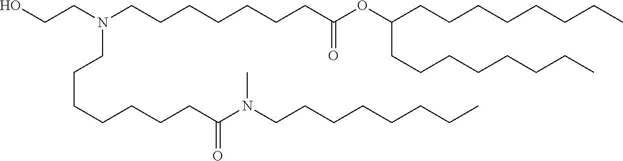 [SMILES: CCCCCCCCC(CCCCCCCC)OC(=O)CCCCCCCN(CCO)CCCCCCCC(=O)N(C)CCCCCCCC] |
| I 146 | 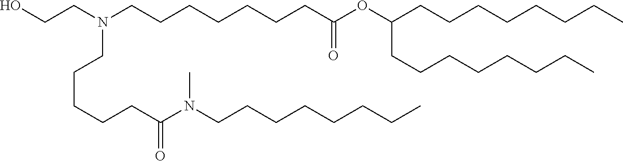 [SMILES: CCCCCCCCC(CCCCCCCC)OC(=O)CCCCCCCN(CCO)CCCCCC(=O)N(C)CCCCCCCC] |
| I 147 | 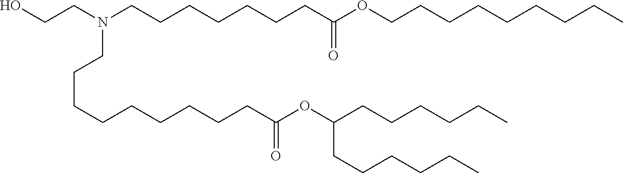 [SMILES: CCCCCCCCCOC(=O)CCCCCCCN(CCO)CCCCCCCCCC(=O)OC(CCCCCC)CCCCCC] |
| I 148 | 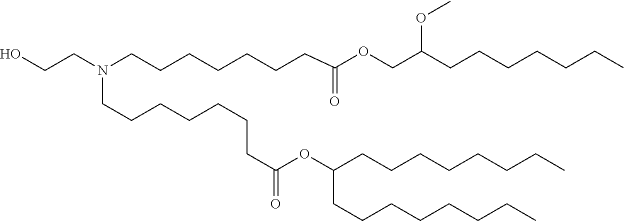 [SMILES: CCCCCCCCC(CCCCCCCC)OC(=O)CCCCCCCN(CCO)CCCCCCCC(=O)OCC(CCCCCCC)OC] |
| I 149 | 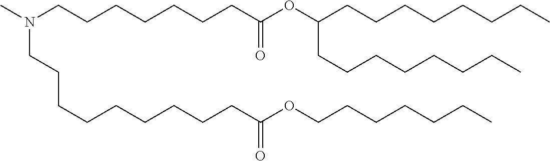 [SMILES: CCCCCCCCC(CCCCCCCC)OC(=O)CCCCCCCN(C)CCCCCCCCCC(=O)OCCCCCCC] |
| I 150 | 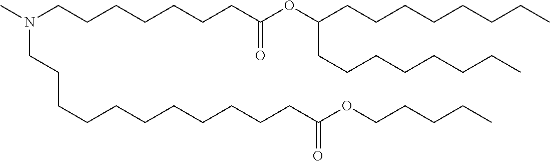 [SMILES: CCCCCCCCC(CCCCCCCC)OC(=O)CCCCCCCN(C)CCCCCCCCCCCC(=O)OCCCCC] |
| I 151 | 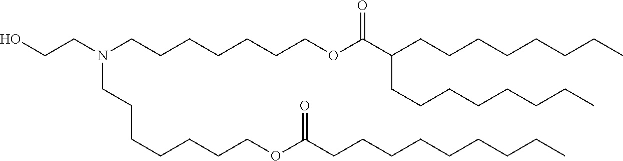 [SMILES: CCCCCCCCCC(=O)OCCCCCCCN(CCO)CCCCCCCOC(=O)C(CCCCCCCC)CCCCCCCC] |
| I 152 | 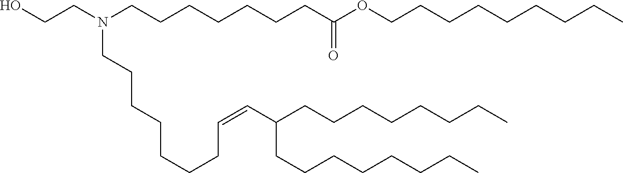 [SMILES: CCCCCCCCCOC(=O)CCCCCCCN(CCO)CCCCCCC/C=C\C(CCCCCCCC)CCCCCCCC] |
| I 153 | 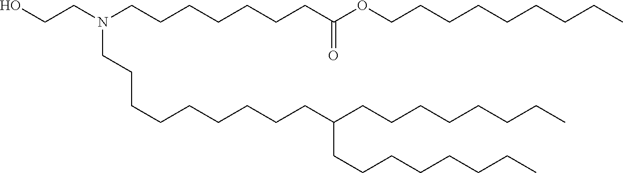 [SMILES: CCCCCCCCCOC(=O)CCCCCCCN(CCO)CCCCCCCCCC(CCCCCCCC)CCCCCCCC] |
| I 154 | 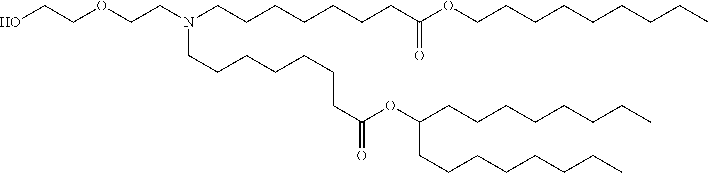 [SMILES: CCCCCCCCCOC(=O)CCCCCCCN(CCCCCCCC(=O)OC(CCCCCCCC)CCCCCCCC)CCOCCO] |
| I 155 | 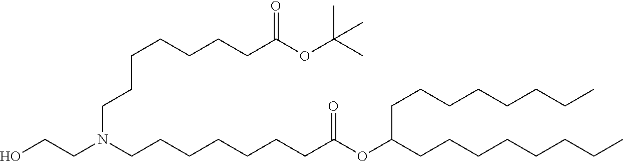 [SMILES: CCCCCCCCC(CCCCCCCC)OC(=O)CCCCCCCN(CCO)CCCCCCCC(=O)OC(C)(C)C] |
| I 156 | 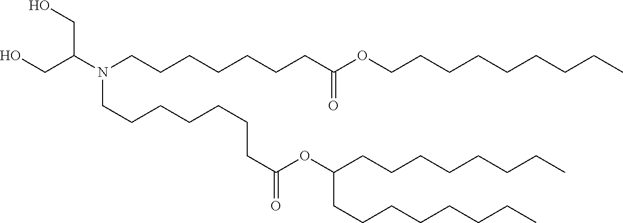 [SMILES: CCCCCCCCCOC(=O)CCCCCCCN(CCCCCCCC(=O)OC(CCCCCCCC)CCCCCCCC)C(CO)CO] |
| I 157 | 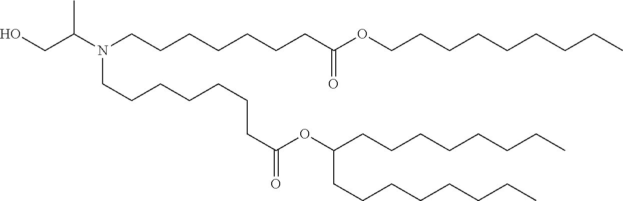 [SMILES: CCCCCCCCCOC(=O)CCCCCCCN(CCCCCCCC(=O)OC(CCCCCCCC)CCCCCCCC)C(C)CO] |
| I 158 | 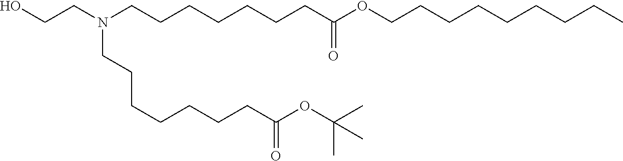 [SMILES: CCCCCCCCCOC(=O)CCCCCCCN(CCO)CCCCCCCC(=O)OC(C)(C)C] |
| I 159 | 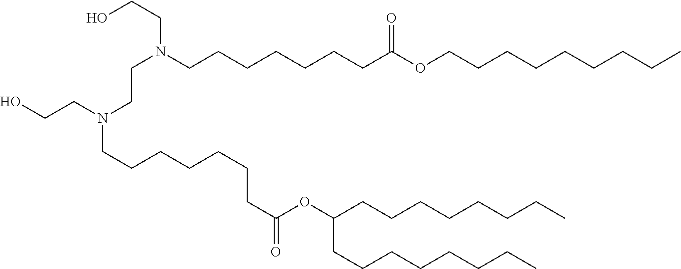 [SMILES: CCCCCCCCCOC(=O)CCCCCCCN(CCO)CCN(CCO)CCCCCCCC(=O)OC(CCCCCCCC)CCCCCCCC] |
| I 160 | 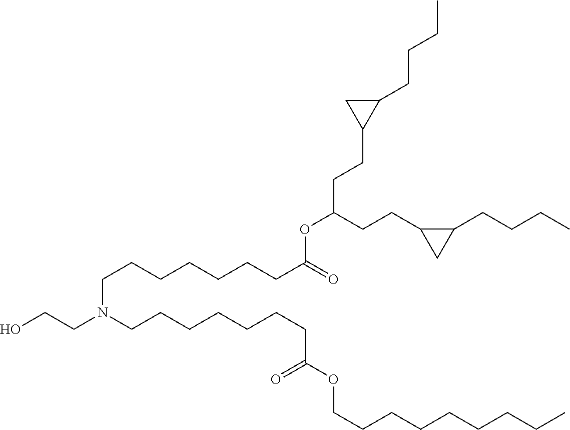 [SMILES: CCCCCCCCCOC(=O)CCCCCCCN(CCO)CCCCCCCC(=O)OC(CCC1CC1CCCC)CCC1CC1CCCC] |
| I 161 | 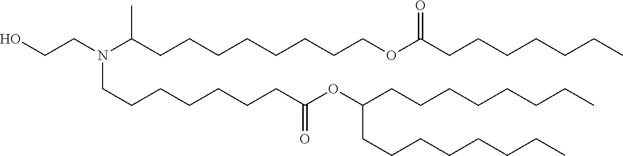 [SMILES: CCCCCCCCC(CCCCCCCC)OC(=O)CCCCCCCN(CCO)C(C)CCCCCCCCOC(=O)CCCCCCC] |
| I 162 | 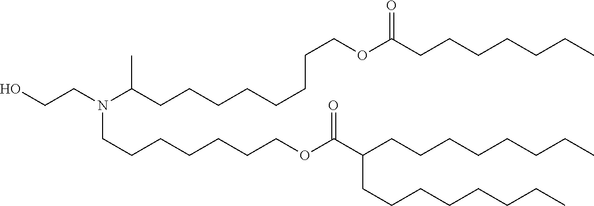 [SMILES: CCCCCCCCC(CCCCCCCC)C(=O)OCCCCCCCN(CCO)C(C)CCCCCCCCOC(=O)CCCCCCC] |
| I 163 | 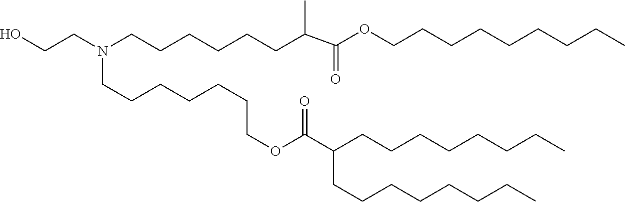 [SMILES: CCCCCCCCCOC(=O)C(C)CCCCCCN(CCO)CCCCCCCOC(=O)C(CCCCCCCC)CCCCCCCC] |
| I 164 | 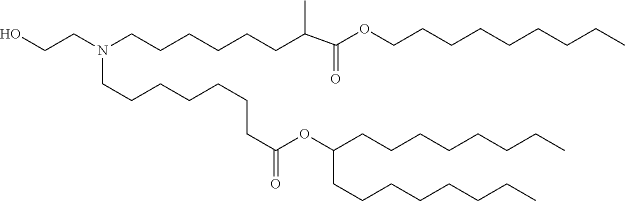 [SMILES: CCCCCCCCCOC(=O)C(C)CCCCCCN(CCO)CCCCCCCC(=O)OC(CCCCCCCC)CCCCCCCC] |
| I 165 | 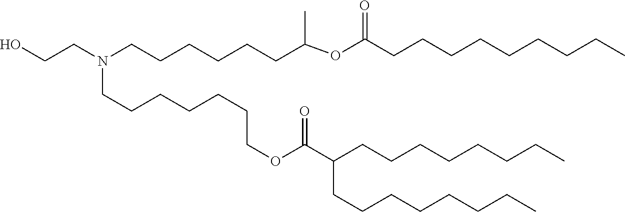 [SMILES: CCCCCCCCCC(=O)OC(C)CCCCCCN(CCO)CCCCCCCOC(=O)C(CCCCCCCC)CCCCCCCC] |
| I 166 | 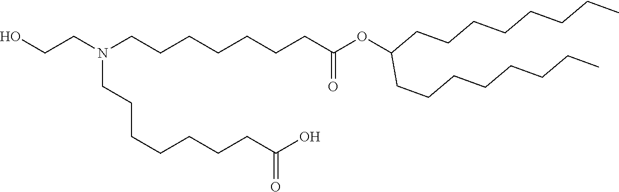 [SMILES: CCCCCCCCC(CCCCCCCC)OC(=O)CCCCCCCN(CCO)CCCCCCCC(=O)O] |
| I 167 | 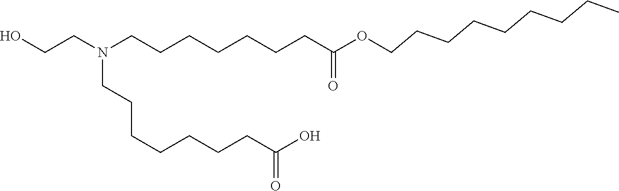 [SMILES: CCCCCCCCCOC(=O)CCCCCCCN(CCO)CCCCCCCC(=O)O] |
| I 168 | 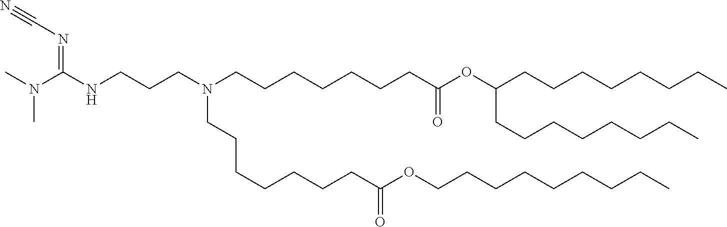 [SMILES: CCCCCCCCCOC(=O)CCCCCCCN(CCCCCCCC(=O)OC(CCCCCCCC)CCCCCCCC)CCCN/C(=N/C#N)N(C)C] |
| I 169 | 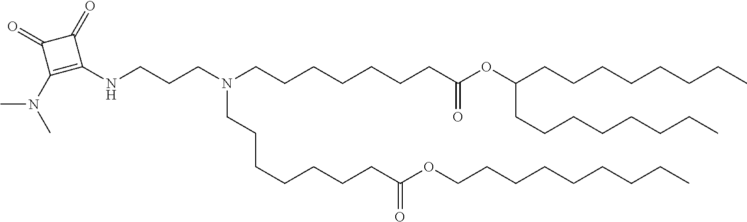 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I 170 | 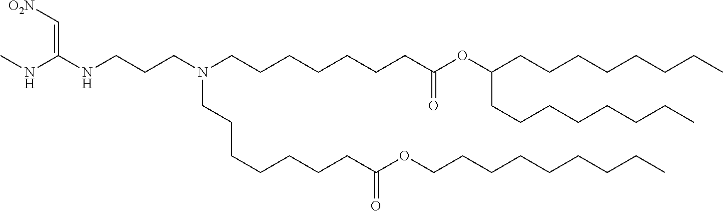 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I 171 | 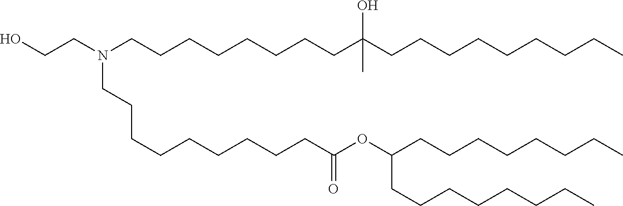 [SMILES: CCCCCCCCCC(C)(O)CCCCCCCCN(CCO)CCCCCCCCCC(=O)OC(CCCCCCCC)CCCCCCCC] |
| I 172 | 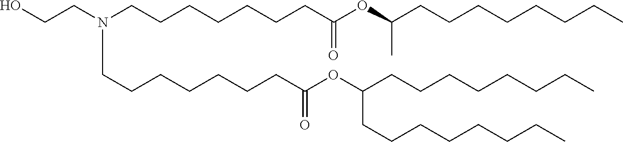 [SMILES: CCCCCCCCC(CCCCCCCC)OC(=O)CCCCCCCN(CCO)CCCCCCCC(=O)O[C@H](C)CCCCCCCC] |
| I 173 | 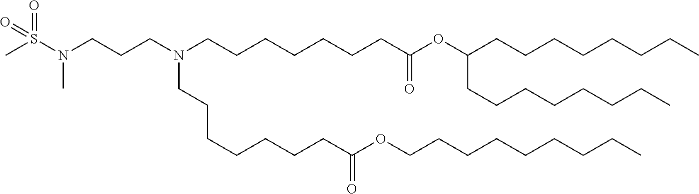 [SMILES: CCCCCCCCCOC(=O)CCCCCCCN(CCCCCCCC(=O)OC(CCCCCCCC)CCCCCCCC)CCCN(C)S(C)(=O)=O] |
| I 174 | 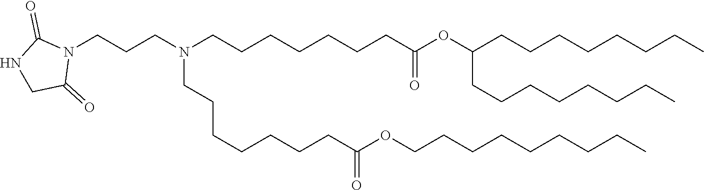 [SMILES: CCCCCCCCCOC(=O)CCCCCCCN(CCCCCCCC(=O)OC(CCCCCCCC)CCCCCCCC)CCCN1C(=O)CNC1=O] |
| I 175 | 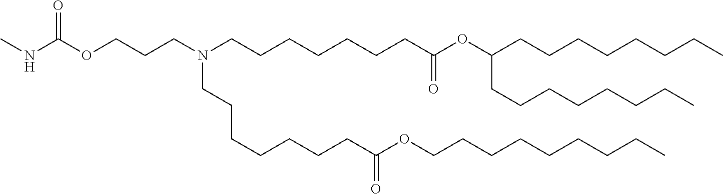 [SMILES: CCCCCCCCCOC(=O)CCCCCCCN(CCCCCCCC(=O)OC(CCCCCCCC)CCCCCCCC)CCCOC(=O)NC] |
| I 176 | 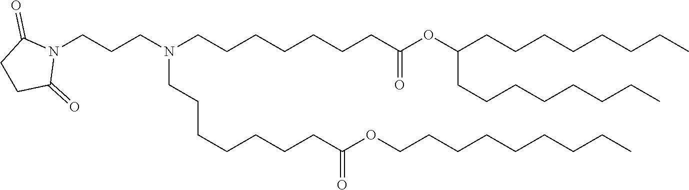 [SMILES: CCCCCCCCCOC(=O)CCCCCCCN(CCCCCCCC(=O)OC(CCCCCCCC)CCCCCCCC)CCCN1C(=O)CCC1=O] |
| I 177 | 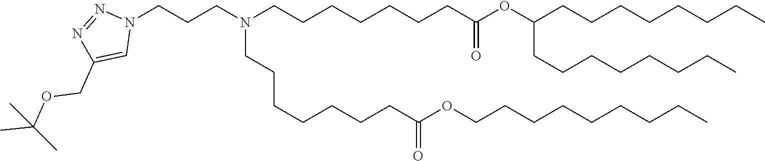 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I 178 | 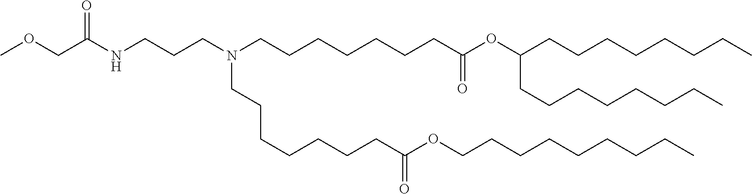 [SMILES: CCCCCCCCCOC(=O)CCCCCCCN(CCCCCCCC(=O)OC(CCCCCCCC)CCCCCCCC)CCCNC(=O)COC] |
| I 179 | 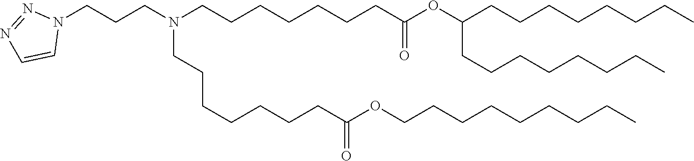 [SMILES: CCCCCCCCCOC(=O)CCCCCCCN(CCCCCCCC(=O)OC(CCCCCCCC)CCCCCCCC)CCCn1ccnn1] |
| I 180 | 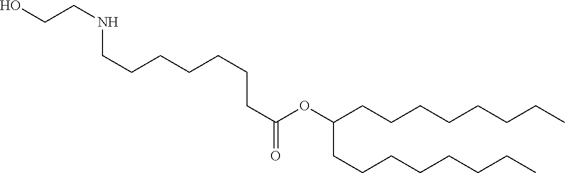 [SMILES: CCCCCCCCC(CCCCCCCC)OC(=O)CCCCCCCNCCO] |
| I 181 | 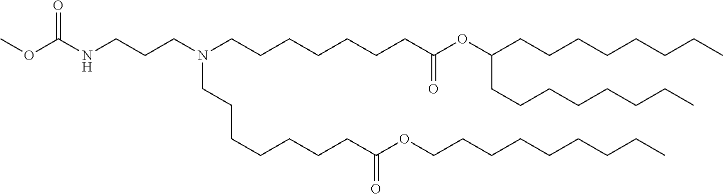 [SMILES: CCCCCCCCCOC(=O)CCCCCCCN(CCCCCCCC(=O)OC(CCCCCCCC)CCCCCCCC)CCCNC(=O)OC] |
| I 182 | 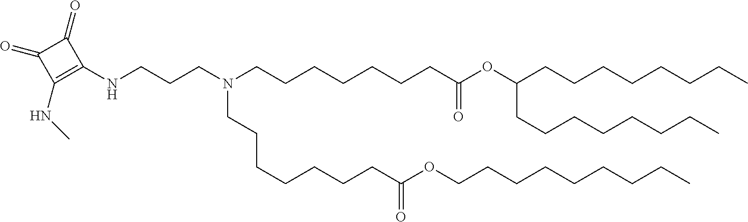 [SMILES: CCCCCCCCCOC(=O)CCCCCCCN(CCCCCCCC(=O)OC(CCCCCCCC)CCCCCCCC)CCCNc1c(NC)c(=O)c1=O] |
| I 183 | 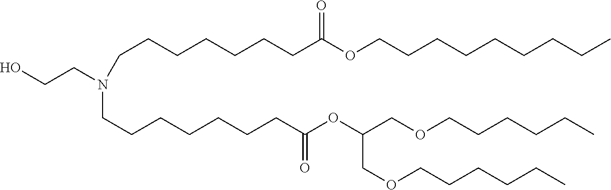 [SMILES: CCCCCCCCCOC(=O)CCCCCCCN(CCO)CCCCCCCC(=O)OC(COCCCCCC)COCCCCCC] |
| I 184 | 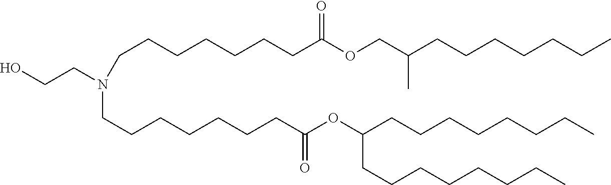 [SMILES: CCCCCCCCC(CCCCCCCC)OC(=O)CCCCCCCN(CCO)CCCCCCCC(=O)OCC(C)CCCCCCC] |
| I 185 | 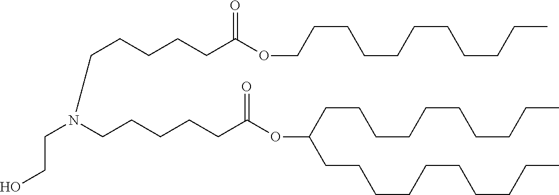 [SMILES: CCCCCCCCCCCOC(=O)CCCCCN(CCO)CCCCCC(=O)OC(CCCCCCCCCC)CCCCCCCCCC] |
| I 186 | 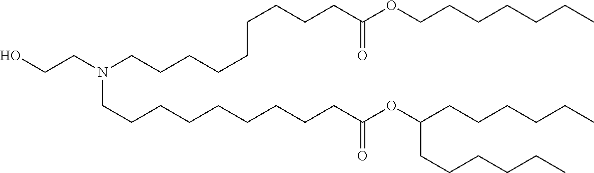 [SMILES: CCCCCCCOC(=O)CCCCCCCCCN(CCO)CCCCCCCCCC(=O)OC(CCCCCC)CCCCCC] |
| I 187 | 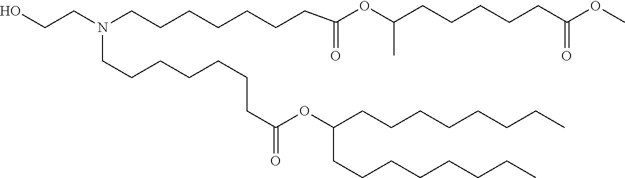 [SMILES: CCCCCCCCC(CCCCCCCC)OC(=O)CCCCCCCN(CCO)CCCCCCCC(=O)OC(C)CCCCCC(=O)OC] |
| I 188 | 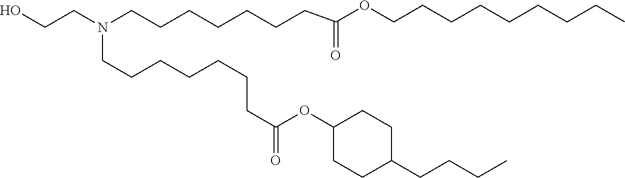 [SMILES: CCCCCCCCCOC(=O)CCCCCCCN(CCO)CCCCCCCC(=O)OC1CCC(CCCC)CC1] |
| I 189 | 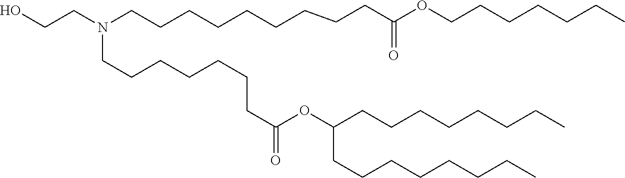 [SMILES: CCCCCCCCC(CCCCCCCC)OC(=O)CCCCCCCN(CCO)CCCCCCCCCC(=O)OCCCCCCC] |
| I 190 | 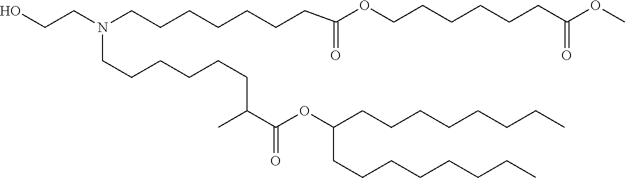 [SMILES: CCCCCCCCC(CCCCCCCC)OC(=O)C(C)CCCCCCN(CCO)CCCCCCCC(=O)OCCCCCCC(=O)OC] |
| I 191 | 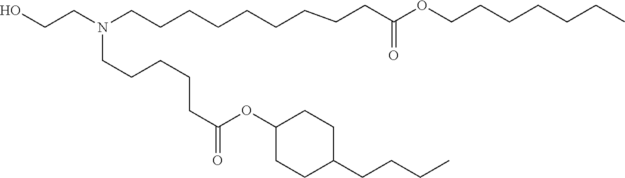 [SMILES: CCCCCCCOC(=O)CCCCCCCCCN(CCO)CCCCCC(=O)OC1CCC(CCCC)CC1] |
| I 192 | 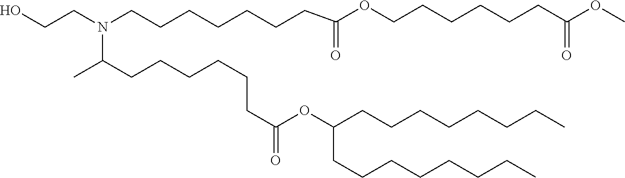 [SMILES: CCCCCCCCC(CCCCCCCC)OC(=O)CCCCCCC(C)N(CCO)CCCCCCCC(=O)OCCCCCCC(=O)OC] |
| I 193 | 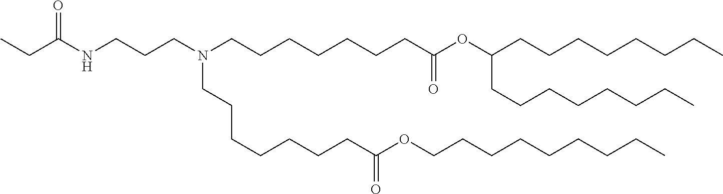 [SMILES: CCCCCCCCCOC(=O)CCCCCCCN(CCCCCCCC(=O)OC(CCCCCCCC)CCCCCCCC)CCCNC(=O)CC] |
| I 194 | 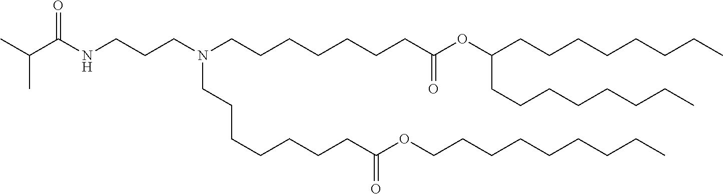 [SMILES: CCCCCCCCCOC(=O)CCCCCCCN(CCCCCCCC(=O)OC(CCCCCCCC)CCCCCCCC)CCCNC(=O)C(C)C] |
| I 195 | 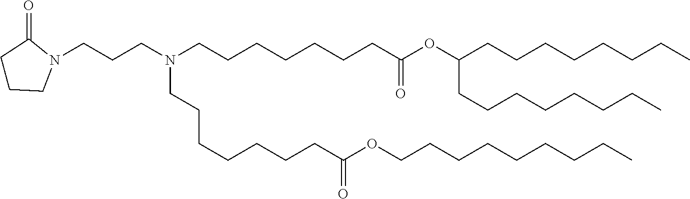 [SMILES: CCCCCCCCCOC(=O)CCCCCCCN(CCCCCCCC(=O)OC(CCCCCCCC)CCCCCCCC)CCCN1CCCC1=O] |
| I 196 | 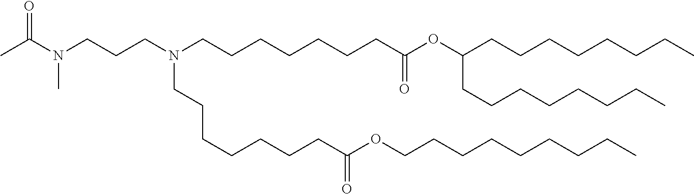 [SMILES: CCCCCCCCCOC(=O)CCCCCCCN(CCCCCCCC(=O)OC(CCCCCCCC)CCCCCCCC)CCCN(C)C(C)=O] |
| I 197 | 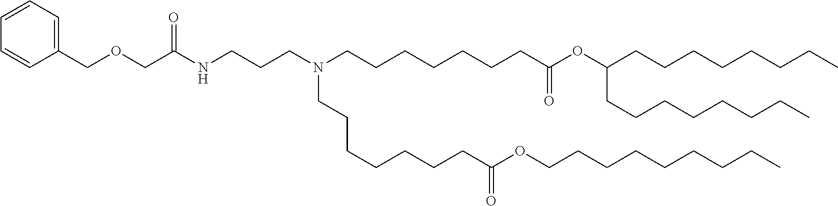 [SMILES: CCCCCCCCCOC(=O)CCCCCCCN(CCCCCCCC(=O)OC(CCCCCCCC)CCCCCCCC)CCCNC(=O)COCc1ccccc1] |
| I 198 | 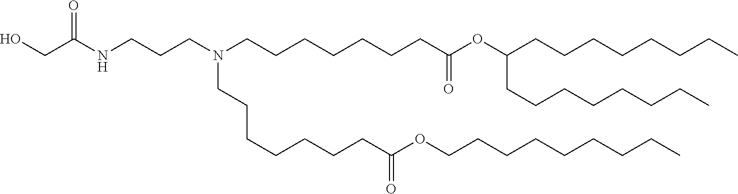 [SMILES: CCCCCCCCCOC(=O)CCCCCCCN(CCCCCCCC(=O)OC(CCCCCCCC)CCCCCCCC)CCCNC(=O)CO] |
| I 199 | 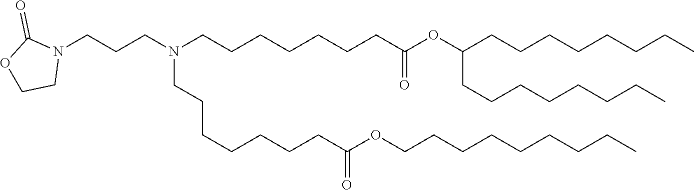 [SMILES: CCCCCCCCCOC(=O)CCCCCCCN(CCCCCCCC(=O)OC(CCCCCCCC)CCCCCCCC)CCCN1CCOC1=O] |
| I 200 | 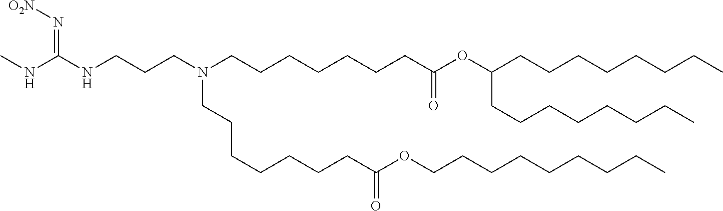 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I 201 | 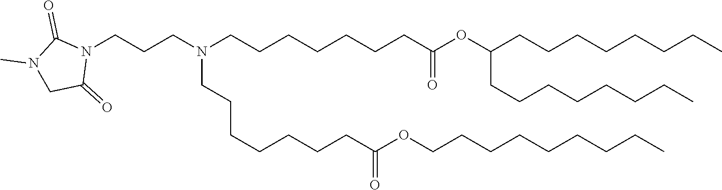 [SMILES: CCCCCCCCCOC(=O)CCCCCCCN(CCCCCCCC(=O)OC(CCCCCCCC)CCCCCCCC)CCCN1C(=O)CN(C)C1=O] |
| I 202 | 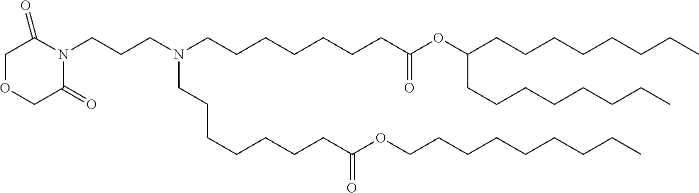 [SMILES: CCCCCCCCCOC(=O)CCCCCCCN(CCCCCCCC(=O)OC(CCCCCCCC)CCCCCCCC)CCCN1C(=O)COCC1=O] |
| I 203 | 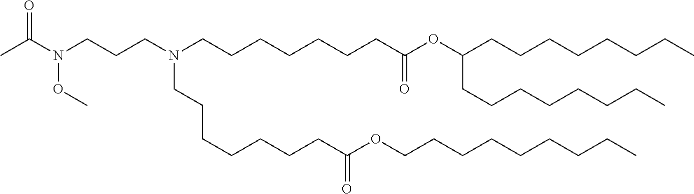 [SMILES: CCCCCCCCCOC(=O)CCCCCCCN(CCCCCCCC(=O)OC(CCCCCCCC)CCCCCCCC)CCCN(OC)C(C)=O] |
| I 204 | 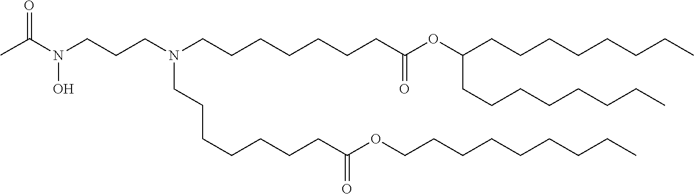 [SMILES: CCCCCCCCCOC(=O)CCCCCCCN(CCCCCCCC(=O)OC(CCCCCCCC)CCCCCCCC)CCCN(O)C(C)=O] |
| I 205 | 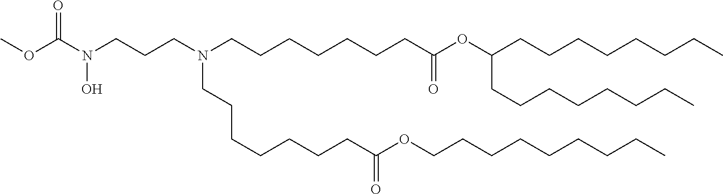 [SMILES: CCCCCCCCCOC(=O)CCCCCCCN(CCCCCCCC(=O)OC(CCCCCCCC)CCCCCCCC)CCCN(O)C(=O)OC] |
| I 206 | 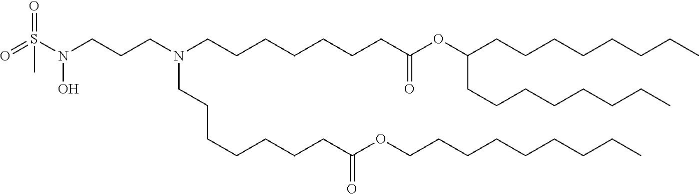 [SMILES: CCCCCCCCCOC(=O)CCCCCCCN(CCCCCCCC(=O)OC(CCCCCCCC)CCCCCCCC)CCCN(O)S(C)(=O)=O] |
| I 207 | 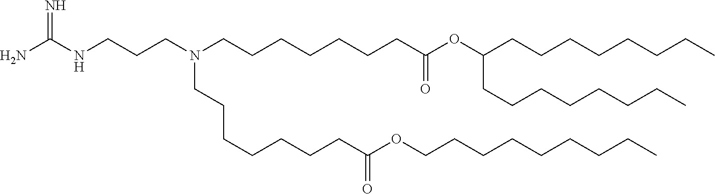 [SMILES: CCCCCCCCCOC(=O)CCCCCCCN(CCCCCCCC(=O)OC(CCCCCCCC)CCCCCCCC)CCCNC(=N)N] |
| I 208 | 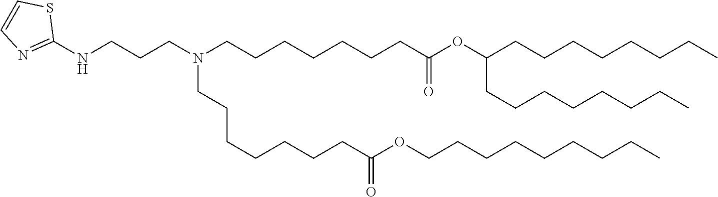 [SMILES: CCCCCCCCCOC(=O)CCCCCCCN(CCCCCCCC(=O)OC(CCCCCCCC)CCCCCCCC)CCCNc1nccs1] |
| I 209 | 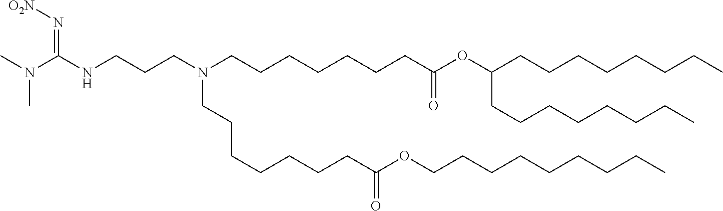 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 210 | 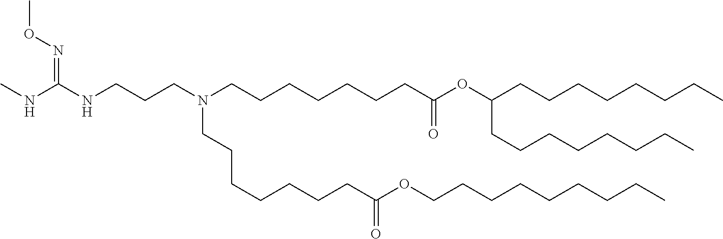 [SMILES: CCCCCCCCCOC(=O)CCCCCCCN(CCCCCCCC(=O)OC(CCCCCCCC)CCCCCCCC)CCCN/C(=N/OC)NC] |
| I 211 | 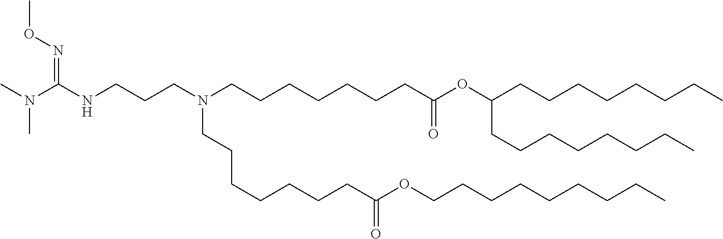 [SMILES: CCCCCCCCCOC(=O)CCCCCCCN(CCCCCCCC(=O)OC(CCCCCCCC)CCCCCCCC)CCCN/C(=N/OC)N(C)C] |
| I 212 | 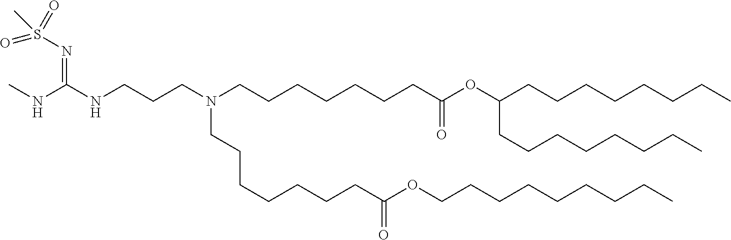 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I 213 | 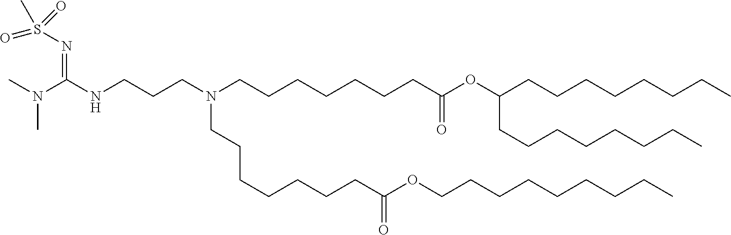 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 214 | 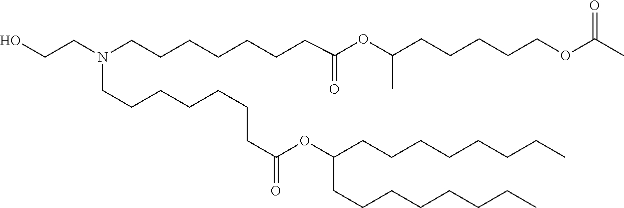 [SMILES: CCCCCCCCC(CCCCCCCC)OC(=O)CCCCCCCN(CCO)CCCCCCCC(=O)OC(C)CCCCCOC(C)=O] |
| I 215 | 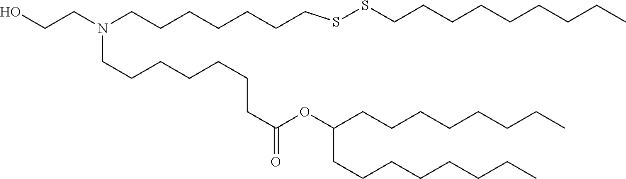 [SMILES: CCCCCCCCCSSCCCCCCCN(CCO)CCCCCCCC(=O)OC(CCCCCCCC)CCCCCCCC] |
| I 216 | 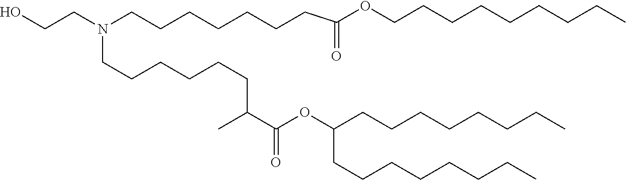 [SMILES: CCCCCCCCCOC(=O)CCCCCCCN(CCO)CCCCCCC(C)C(=O)OC(CCCCCCCC)CCCCCCCC] |
| I 217 | 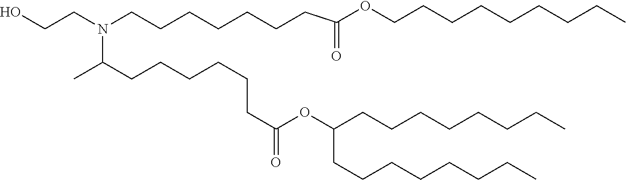 [SMILES: CCCCCCCCCOC(=O)CCCCCCCN(CCO)C(C)CCCCCCC(=O)OC(CCCCCCCC)CCCCCCCC] |
| I 218 | 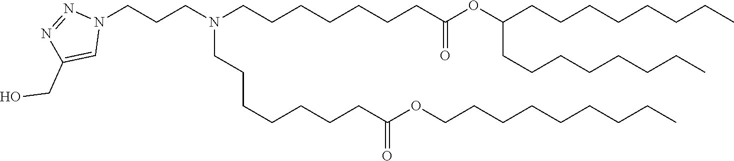 [SMILES: CCCCCCCCCOC(=O)CCCCCCCN(CCCCCCCC(=O)OC(CCCCCCCC)CCCCCCCC)CCCn1cc(CO)nn1] |
| I 219 | 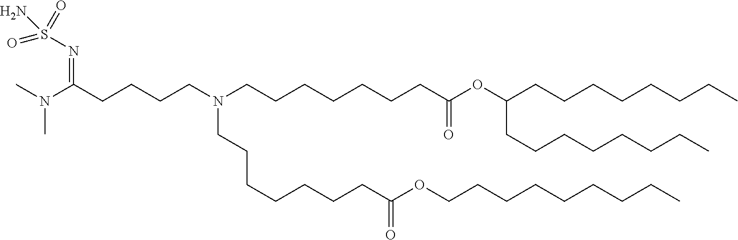 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I 220 | 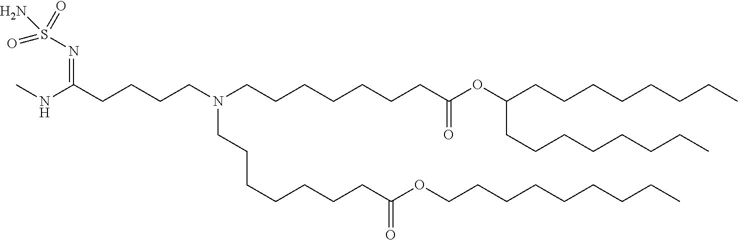 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I 221 | 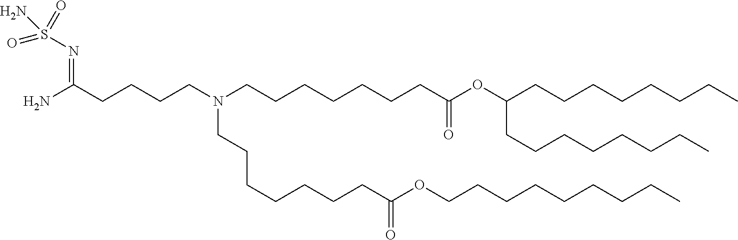 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I 222 | 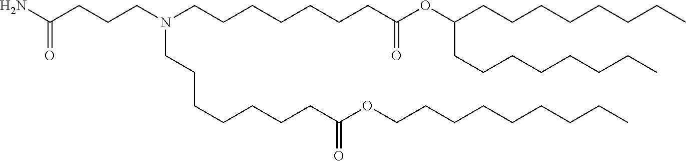 [SMILES: CCCCCCCCCOC(=O)CCCCCCCN(CCCCCCCC(=O)OC(CCCCCCCC)CCCCCCCC)CCCC(N)=O] |
| I 223 | 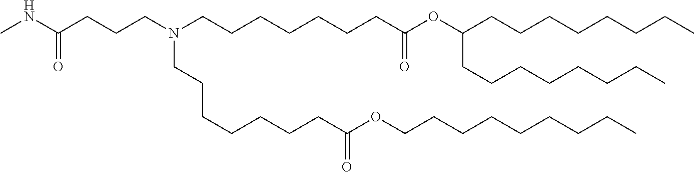 [SMILES: CCCCCCCCCOC(=O)CCCCCCCN(CCCCCCCC(=O)OC(CCCCCCCC)CCCCCCCC)CCCC(=O)NC] |
| I 224 | 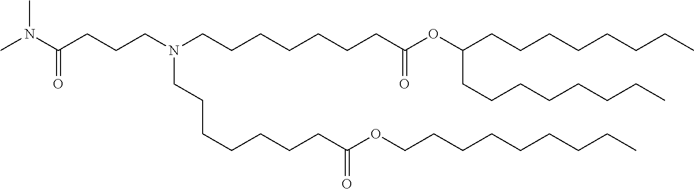 [SMILES: CCCCCCCCCOC(=O)CCCCCCCN(CCCCCCCC(=O)OC(CCCCCCCC)CCCCCCCC)CCCC(=O)N(C)C] |
| I 225 | 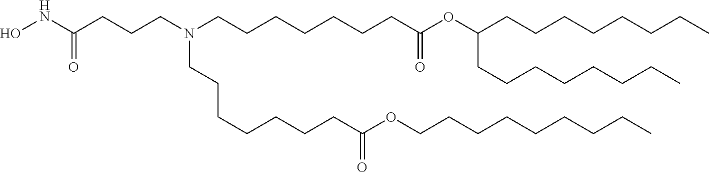 [SMILES: CCCCCCCCCOC(=O)CCCCCCCN(CCCCCCCC(=O)OC(CCCCCCCC)CCCCCCCC)CCCC(=O)NO] |
| I 226 | 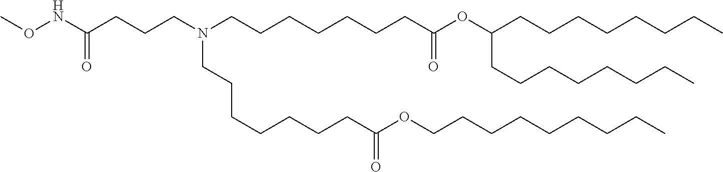 [SMILES: CCCCCCCCCOC(=O)CCCCCCCN(CCCCCCCC(=O)OC(CCCCCCCC)CCCCCCCC)CCCC(=O)NOC] |
| I 227 | 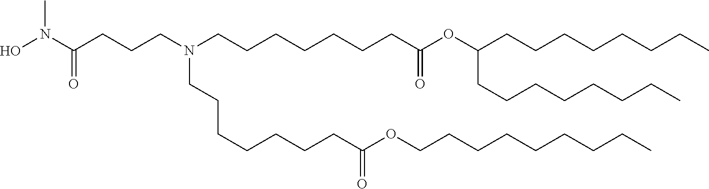 [SMILES: CCCCCCCCCOC(=O)CCCCCCCN(CCCCCCCC(=O)OC(CCCCCCCC)CCCCCCCC)CCCC(=O)N(C)O] |
| I 228 | 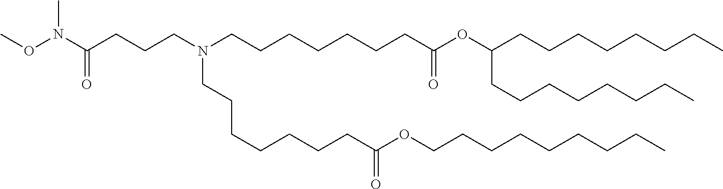 [SMILES: CCCCCCCCCOC(=O)CCCCCCCN(CCCCCCCC(=O)OC(CCCCCCCC)CCCCCCCC)CCCC(=O)N(C)OC] |
| I 229 | 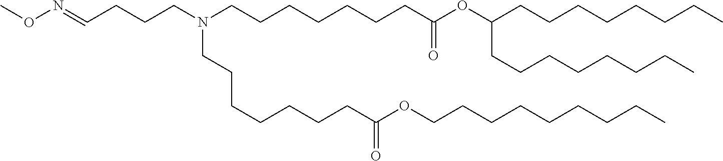 [SMILES: CCCCCCCCCOC(=O)CCCCCCCN(CCC/C=N/OC)CCCCCCCC(=O)OC(CCCCCCCC)CCCCCCCC] |
| I 230 | 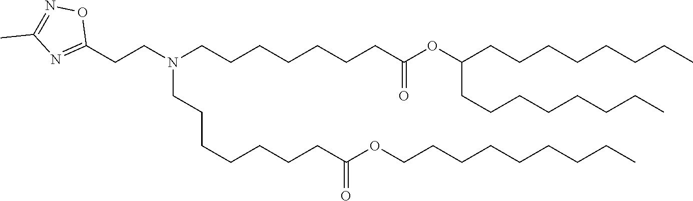 [SMILES: CCCCCCCCCOC(=O)CCCCCCCN(CCCCCCCC(=O)OC(CCCCCCCC)CCCCCCCC)CCc1nc(C)no1] |
| I 231 | 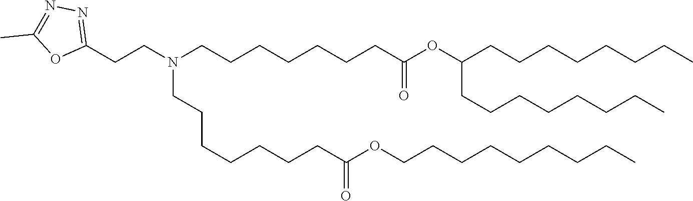 [SMILES: CCCCCCCCCOC(=O)CCCCCCCN(CCCCCCCC(=O)OC(CCCCCCCC)CCCCCCCC)CCc1nnc(C)o1] |
| I 232 | 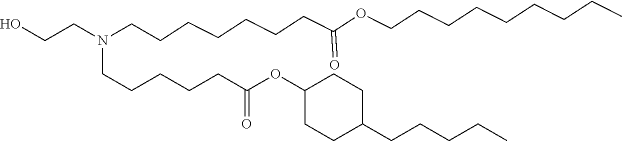 [SMILES: CCCCCCCCCOC(=O)CCCCCCCN(CCO)CCCCCC(=O)OC1CCC(CCCCC)CC1] |
| I 233 | 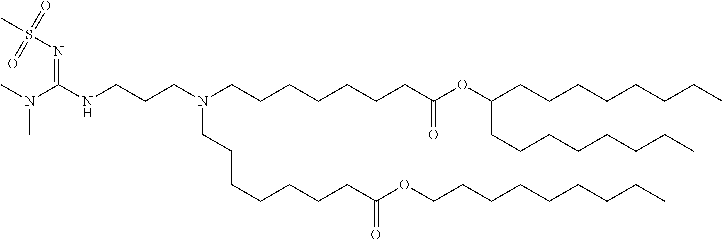 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 234 | 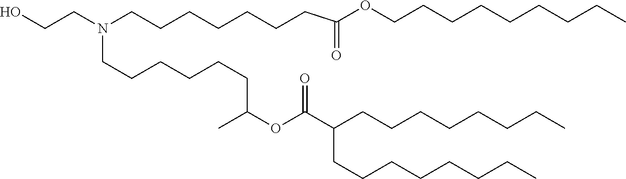 [SMILES: CCCCCCCCCOC(=O)CCCCCCCN(CCO)CCCCCCC(C)OC(=O)C(CCCCCCCC)CCCCCCCC] |
| I 235 | 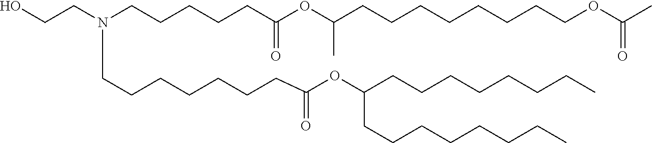 [SMILES: CCCCCCCCC(CCCCCCCC)OC(=O)CCCCCCCN(CCO)CCCCCC(=O)OC(C)CCCCCCCCOC(C)=O] |
| I 236 | 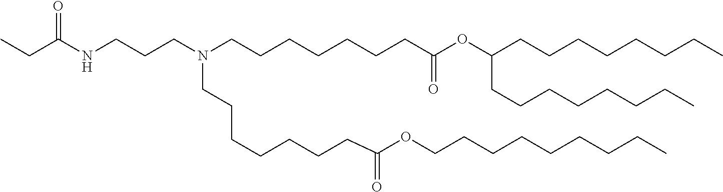 [SMILES: CCCCCCCCCOC(=O)CCCCCCCN(CCCCCCCC(=O)OC(CCCCCCCC)CCCCCCCC)CCCNC(=O)CC] |
| I 237 | 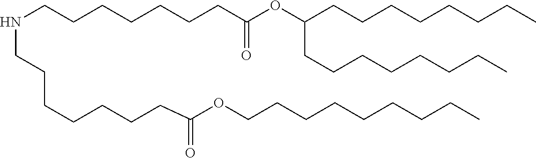 [SMILES: CCCCCCCCCOC(=O)CCCCCCCNCCCCCCCC(=O)OC(CCCCCCCC)CCCCCCCC] |
| I 238 | 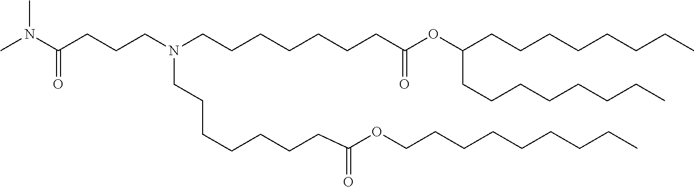 [SMILES: CCCCCCCCCOC(=O)CCCCCCCN(CCCCCCCC(=O)OC(CCCCCCCC)CCCCCCCC)CCCC(=O)N(C)C] |
| I 239 | 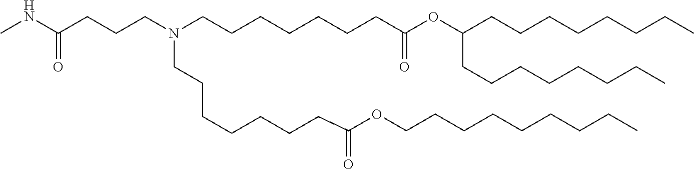 [SMILES: CCCCCCCCCOC(=O)CCCCCCCN(CCCCCCCC(=O)OC(CCCCCCCC)CCCCCCCC)CCCC(=O)NC] |
| I 240 | 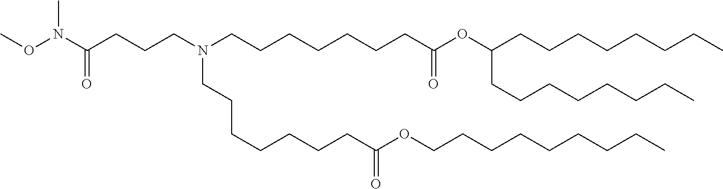 [SMILES: CCCCCCCCCOC(=O)CCCCCCCN(CCCCCCCC(=O)OC(CCCCCCCC)CCCCCCCC)CCCC(=O)N(C)OC] |
| I 241 | 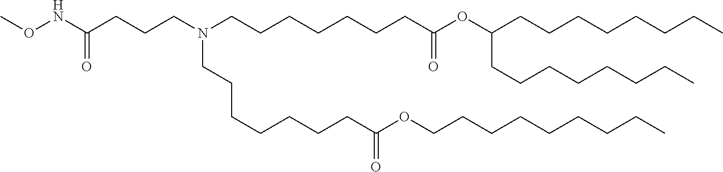 [SMILES: CCCCCCCCCOC(=O)CCCCCCCN(CCCCCCCC(=O)OC(CCCCCCCC)CCCCCCCC)CCCC(=O)NOC] |
| I 242 | 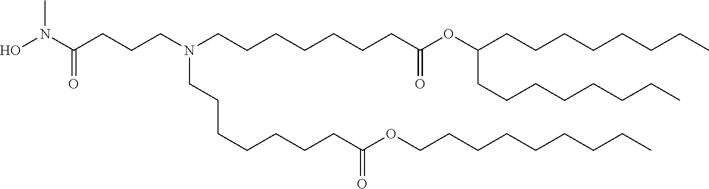 [SMILES: CCCCCCCCCOC(=O)CCCCCCCN(CCCCCCCC(=O)OC(CCCCCCCC)CCCCCCCC)CCCC(=O)N(C)O] |
| I 243 | 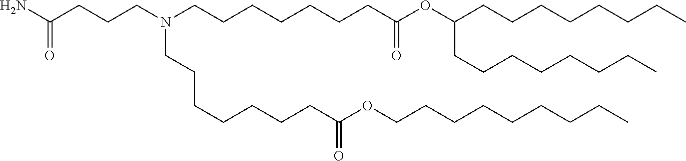 [SMILES: CCCCCCCCCOC(=O)CCCCCCCN(CCCCCCCC(=O)OC(CCCCCCCC)CCCCCCCC)CCCC(N)=O] |
| I 244 | 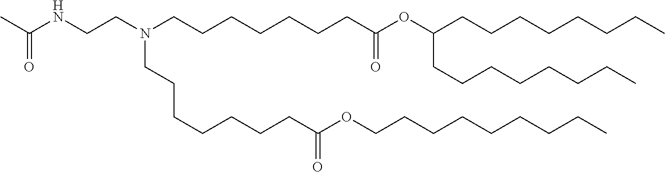 [SMILES: CCCCCCCCCOC(=O)CCCCCCCN(CCCCCCCC(=O)OC(CCCCCCCC)CCCCCCCC)CCNC(C)=O] |
| I 245 | 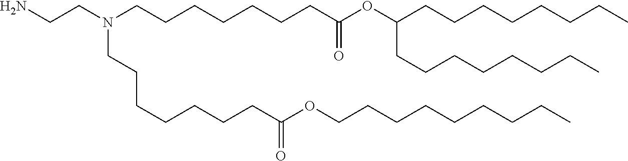 [SMILES: CCCCCCCCCOC(=O)CCCCCCCN(CCN)CCCCCCCC(=O)OC(CCCCCCCC)CCCCCCCC] |
| I 246 | 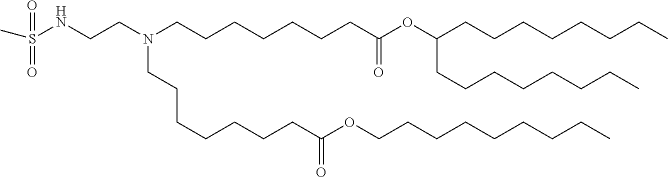 [SMILES: CCCCCCCCCOC(=O)CCCCCCCN(CCCCCCCC(=O)OC(CCCCCCCC)CCCCCCCC)CCNS(C)(=O)=O] |
| I 247 | 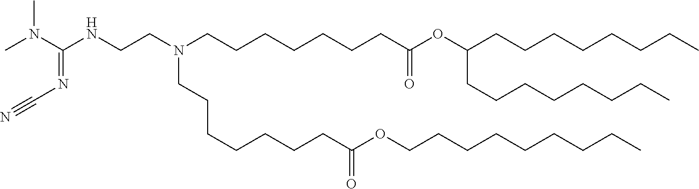 [SMILES: CCCCCCCCCOC(=O)CCCCCCCN(CCCCCCCC(=O)OC(CCCCCCCC)CCCCCCCC)CCN/C(=N/C#N)N(C)C] |
| I 248 | 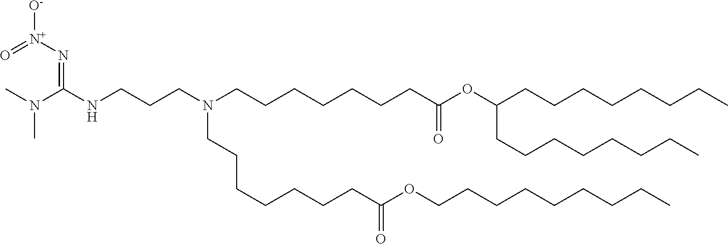 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 249 | 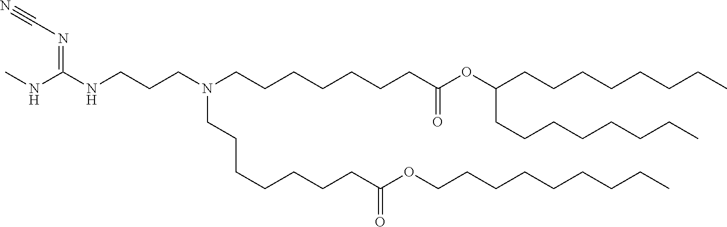 [SMILES: CCCCCCCCCOC(=O)CCCCCCCN(CCCCCCCC(=O)OC(CCCCCCCC)CCCCCCCC)CCCN/C(=N/C#N)NC] |
| I 250 | 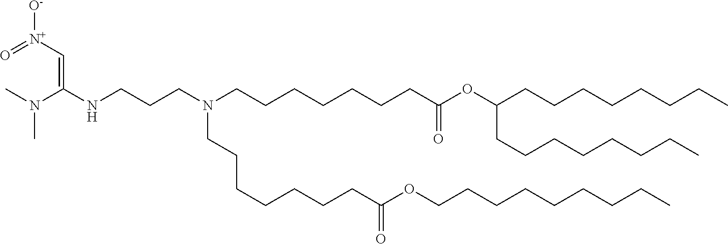 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I 251 | 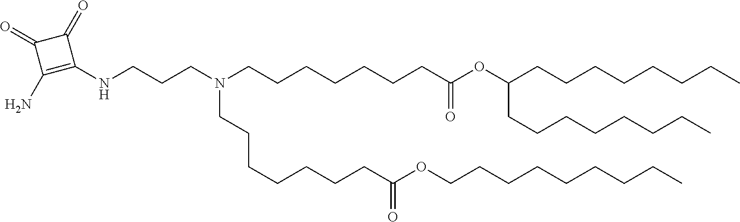 [SMILES: CCCCCCCCCOC(=O)CCCCCCCN(CCCCCCCC(=O)OC(CCCCCCCC)CCCCCCCC)CCCNc1c(N)c(=O)c1=O] |
| I 252 | 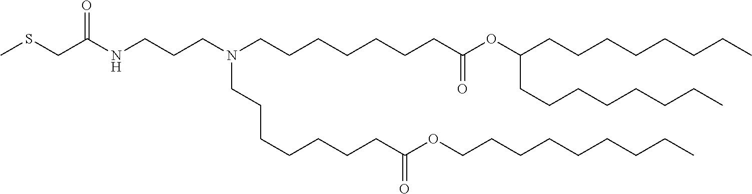 [SMILES: CCCCCCCCCOC(=O)CCCCCCCN(CCCCCCCC(=O)OC(CCCCCCCC)CCCCCCCC)CCCNC(=O)CSC] |
| I 253 | 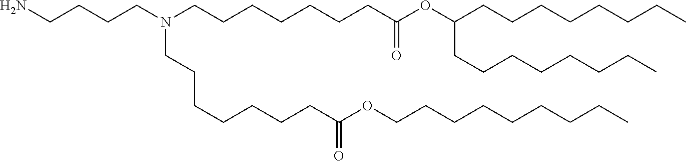 [SMILES: CCCCCCCCCOC(=O)CCCCCCCN(CCCCN)CCCCCCCC(=O)OC(CCCCCCCC)CCCCCCCC] |
| I 254 | 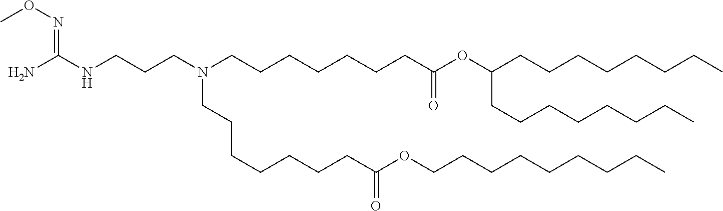 [SMILES: CCCCCCCCCOC(=O)CCCCCCCN(CCCCCCCC(=O)OC(CCCCCCCC)CCCCCCCC)CCCN/C(N)=N/OC] |
| I 255 | 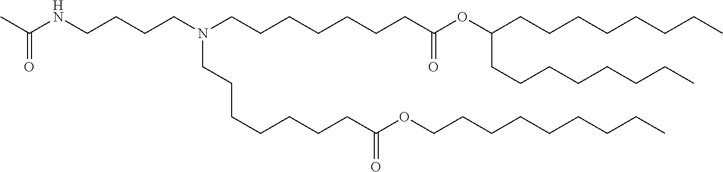 [SMILES: CCCCCCCCCOC(=O)CCCCCCCN(CCCCCCCC(=O)OC(CCCCCCCC)CCCCCCCC)CCCCNC(C)=O] |
| I 256 | 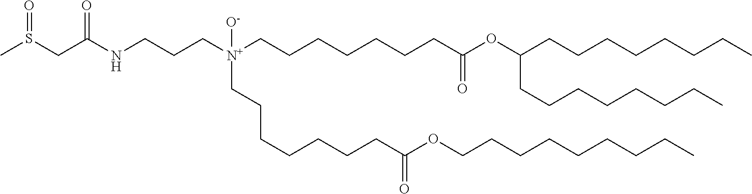 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I 257 | 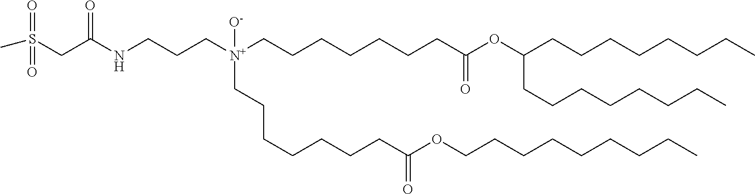 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I 258 | 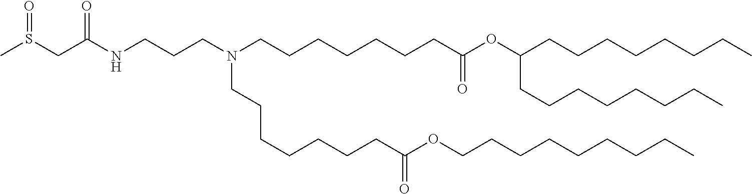 [SMILES: CCCCCCCCCOC(=O)CCCCCCCN(CCCCCCCC(=O)OC(CCCCCCCC)CCCCCCCC)CCCNC(=O)CS(C)=O] |
| I 259 | 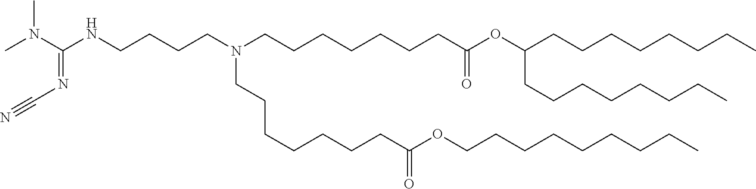 [SMILES: CCCCCCCCCOC(=O)CCCCCCCN(CCCCCCCC(=O)OC(CCCCCCCC)CCCCCCCC)CCCCN/C(=N/C#N)N(C)C] |
| I 260 | 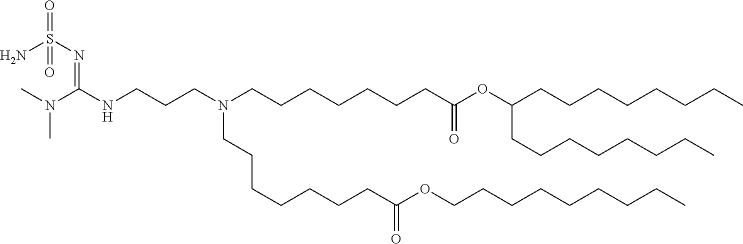 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I 261 | 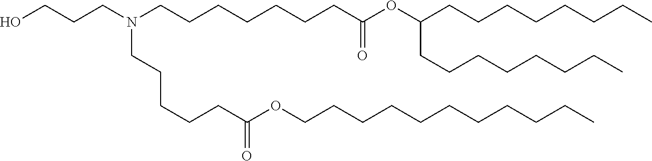 [SMILES: CCCCCCCCCCCOC(=O)CCCCCN(CCCO)CCCCCCCC(=O)OC(CCCCCCCC)CCCCCCCC] |
| I 262 | 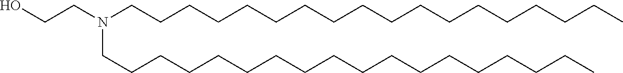 [SMILES: CCCCCCCCCCCCCCCCCCN(CCO)CCCCCCCCCCCCCCCCCC] |
| I 263 | 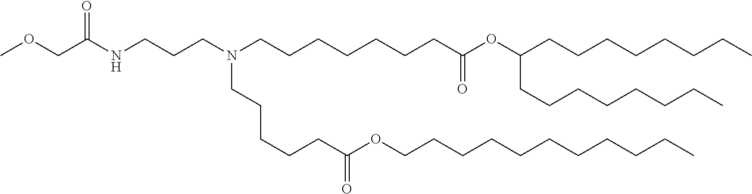 [SMILES: CCCCCCCCCCCOC(=O)CCCCCN(CCCCCCCC(=O)OC(CCCCCCCC)CCCCCCCC)CCCNC(=O)COC] |
| I 264 | 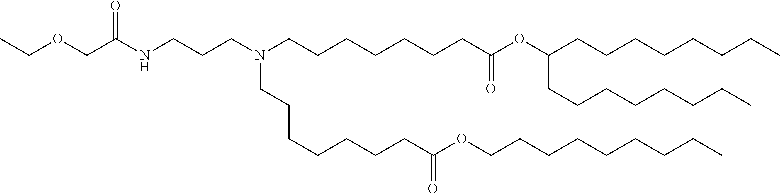 [SMILES: CCCCCCCCCOC(=O)CCCCCCCN(CCCCCCCC(=O)OC(CCCCCCCC)CCCCCCCC)CCCNC(=O)COCC] |
| I 265 | 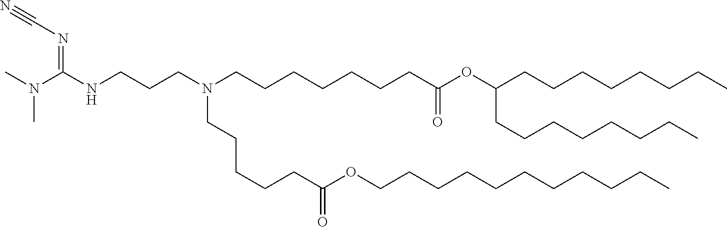 [SMILES: CCCCCCCCCCCOC(=O)CCCCCN(CCCCCCCC(=O)OC(CCCCCCCC)CCCCCCCC)CCCN/C(=N/C#N)N(C)C] |
| I 266 | 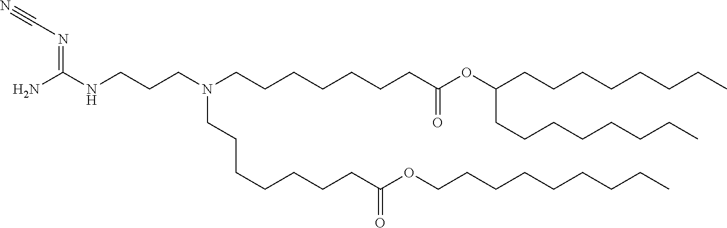 [SMILES: CCCCCCCCCOC(=O)CCCCCCCN(CCCCCCCC(=O)OC(CCCCCCCC)CCCCCCCC)CCCN/C(N)=N/C#N] |
| I 267 | 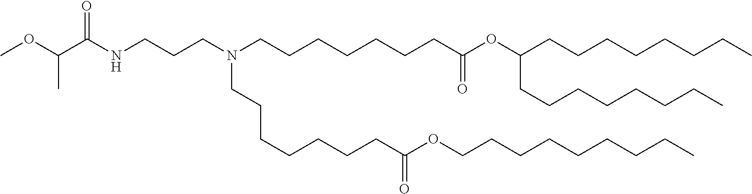 [SMILES: CCCCCCCCCOC(=O)CCCCCCCN(CCCCCCCC(=O)OC(CCCCCCCC)CCCCCCCC)CCCNC(=O)C(C)OC] |
| I 268 | 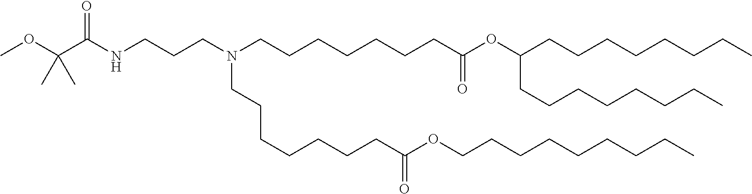 [SMILES: CCCCCCCCCOC(=O)CCCCCCCN(CCCCCCCC(=O)OC(CCCCCCCC)CCCCCCCC)CCCNC(=O)C(C)(C)OC] |
| I 269 | 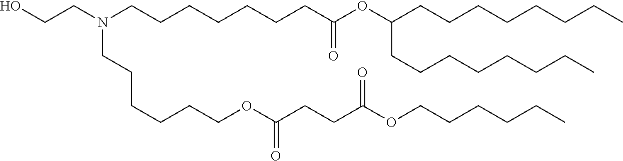 [SMILES: CCCCCCCCC(CCCCCCCC)OC(=O)CCCCCCCN(CCO)CCCCCCOC(=O)CCC(=O)OCCCCCC] |
| I 270 | 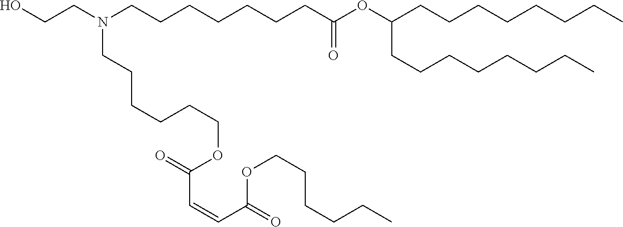 [SMILES: CCCCCCCCC(CCCCCCCC)OC(=O)CCCCCCCN(CCO)CCCCCCOC(=O)/C=C\C(=O)OCCCCCC] |
| I 271 | 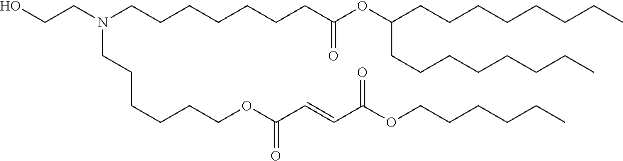 [SMILES: CCCCCCCCC(CCCCCCCC)OC(=O)CCCCCCCN(CCO)CCCCCCOC(=O)/C=C/C(=O)OCCCCCC] |
| I 272 | 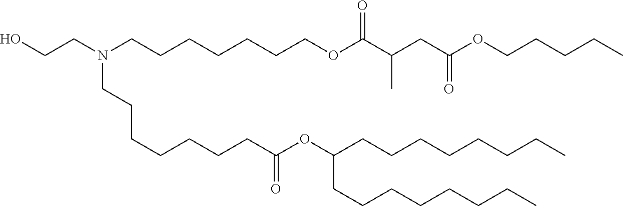 [SMILES: CCCCCCCCC(CCCCCCCC)OC(=O)CCCCCCCN(CCO)CCCCCCCOC(=O)C(C)CC(=O)OCCCCC] |
| I 273 | 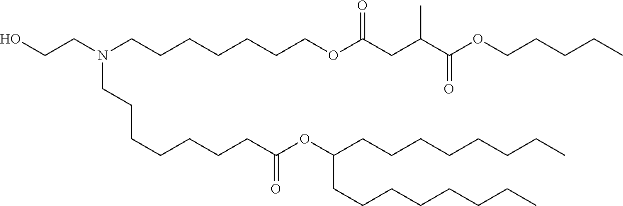 [SMILES: CCCCCCCCC(CCCCCCCC)OC(=O)CCCCCCCN(CCO)CCCCCCCOC(=O)CC(C)C(=O)OCCCCC] |
| I 274 | 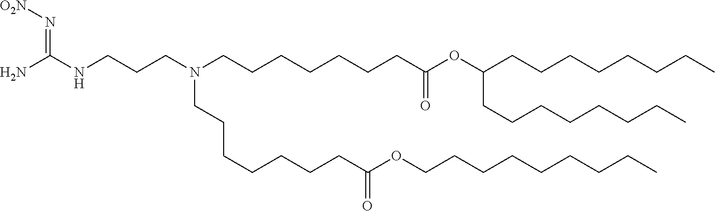 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I 275 | 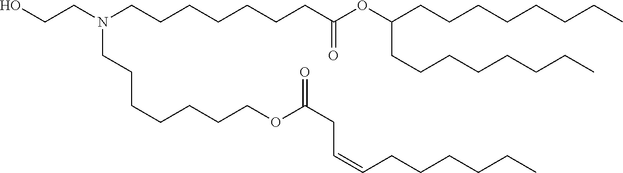 [SMILES: CCCCCC/C=C\CC(=O)OCCCCCCCN(CCO)CCCCCCCC(=O)OC(CCCCCCCC)CCCCCCCC] |
| I 276 | 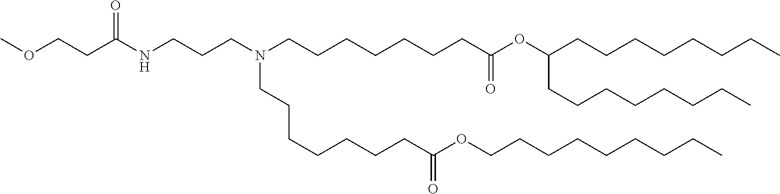 [SMILES: CCCCCCCCCOC(=O)CCCCCCCN(CCCCCCCC(=O)OC(CCCCCCCC)CCCCCCCC)CCCNC(=O)CCOC] |
| I 277 | 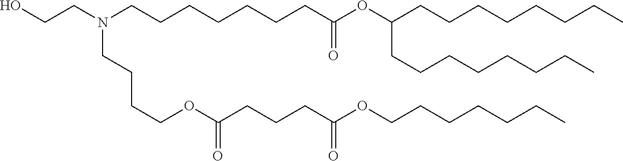 [SMILES: CCCCCCCCC(CCCCCCCC)OC(=O)CCCCCCCN(CCO)CCCCOC(=O)CCCC(=O)OCCCCCCC] |
| I 278 | 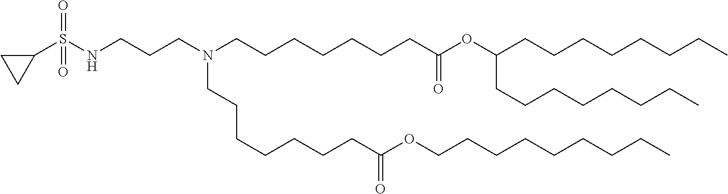 [SMILES: CCCCCCCCCOC(=O)CCCCCCCN(CCCCCCCC(=O)OC(CCCCCCCC)CCCCCCCC)CCCNS(=O)(=O)C1CC1] |
| I 279 | 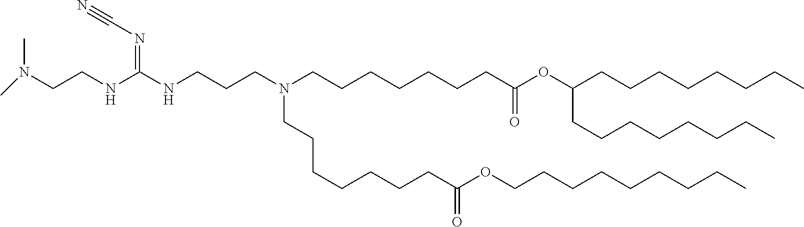 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I 280 | 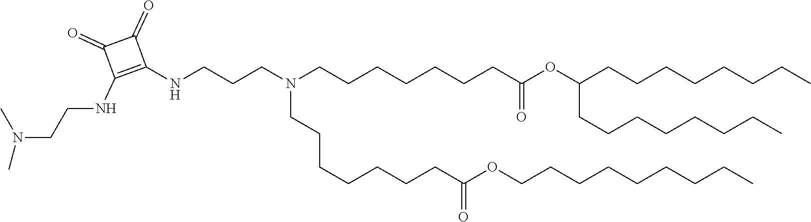 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I 281 | 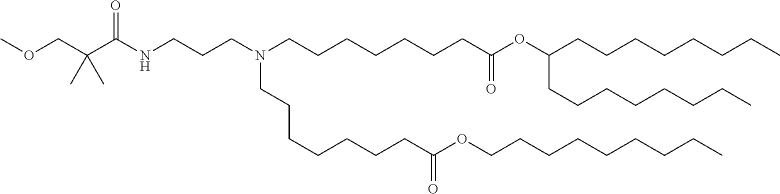 [SMILES: CCCCCCCCCOC(=O)CCCCCCCN(CCCCCCCC(=O)OC(CCCCCCCC)CCCCCCCC)CCCNC(=O)C(C)(C)COC] |
| I 282 | 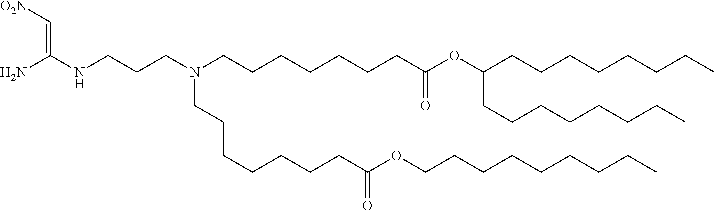 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I 283 | 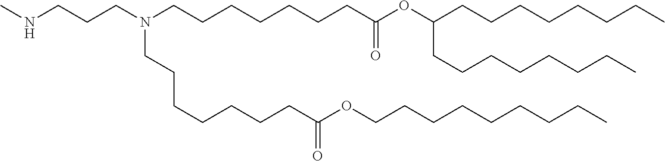 [SMILES: CCCCCCCCCOC(=O)CCCCCCCN(CCCCCCCC(=O)OC(CCCCCCCC)CCCCCCCC)CCCNC] |
| I 284 | 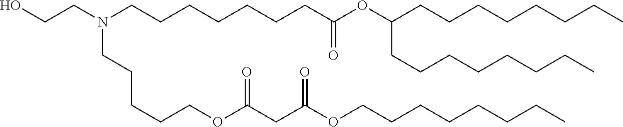 [SMILES: CCCCCCCCOC(=O)CC(=O)OCCCCCN(CCO)CCCCCCCC(=O)OC(CCCCCCCC)CCCCCCCC] |
| I 285 | 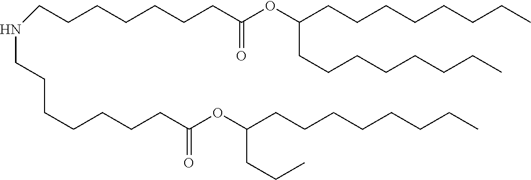 [SMILES: CCCCCCCCC(CCC)OC(=O)CCCCCCCNCCCCCCCC(=O)OC(CCCCCCCC)CCCCCCCC] |
| I 286 | 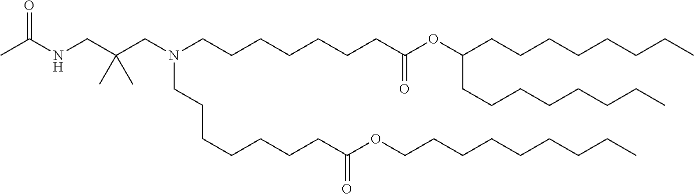 [SMILES: CCCCCCCCCOC(=O)CCCCCCCN(CCCCCCCC(=O)OC(CCCCCCCC)CCCCCCCC)CC(C)(C)CNC(C)=O] |
| I 287 | 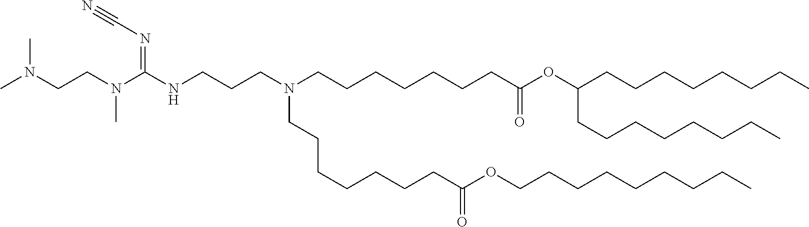 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I 288 | 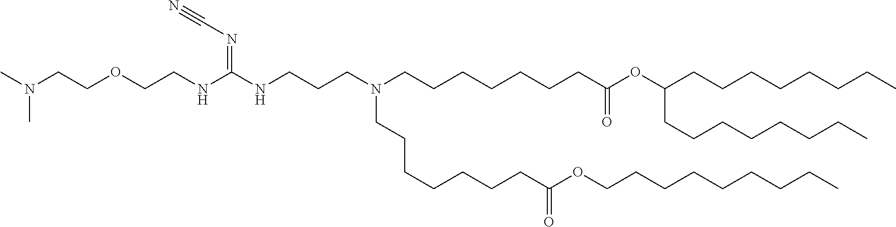 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I 289 | 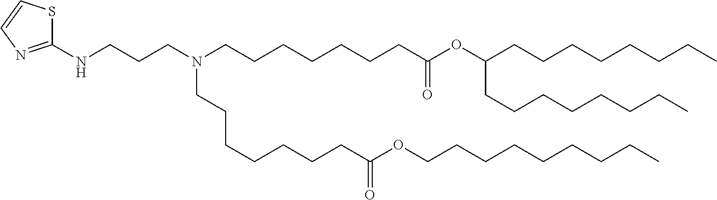 [SMILES: CCCCCCCCCOC(=O)CCCCCCCN(CCCCCCCC(=O)OC(CCCCCCCC)CCCCCCCC)CCCNc1nccs1] |
| I 290 | 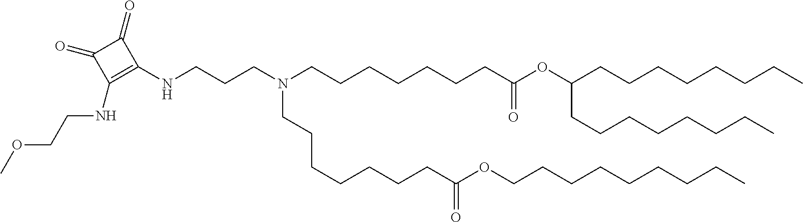 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I 291 | 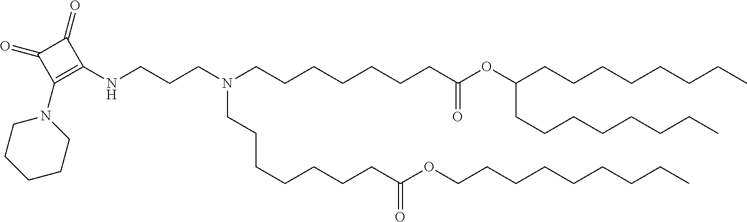 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I 292 | 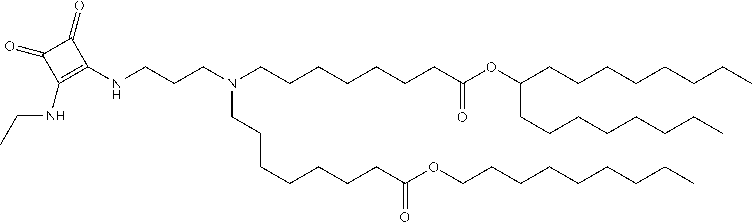 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I 293 | 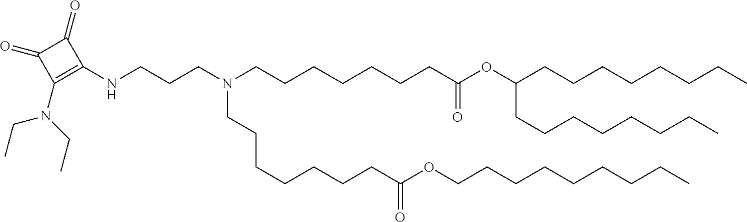 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I 294 | 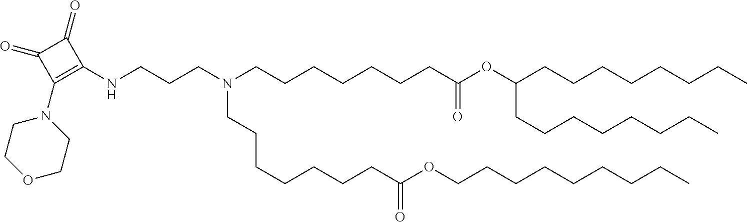 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I 295 | 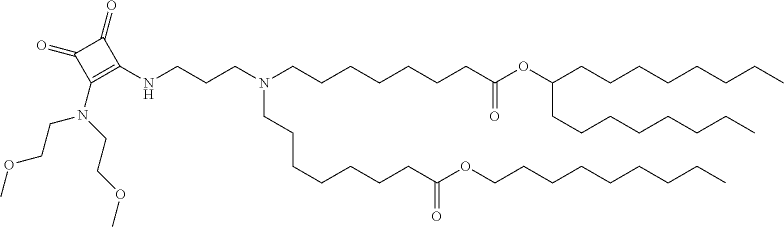 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I 296 | 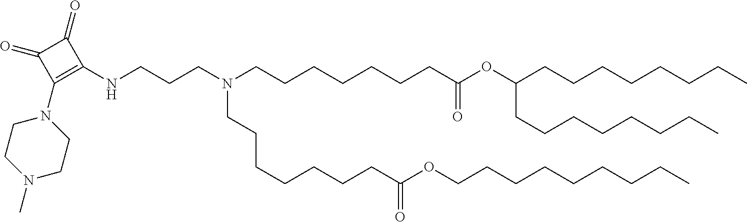 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I 297 | 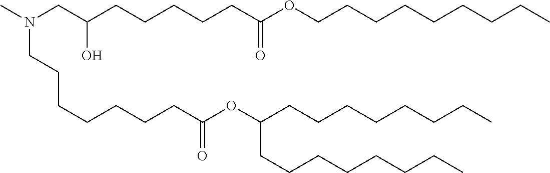 [SMILES: CCCCCCCCCOC(=O)CCCCCC(O)CN(C)CCCCCCCC(=O)OC(CCCCCCCC)CCCCCCCC] |
| I 298 | 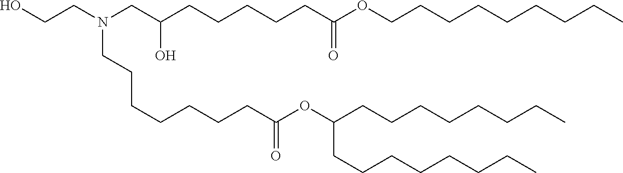 [SMILES: CCCCCCCCCOC(=O)CCCCCC(O)CN(CCO)CCCCCCCC(=O)OC(CCCCCCCC)CCCCCCCC] |
| I 299 | 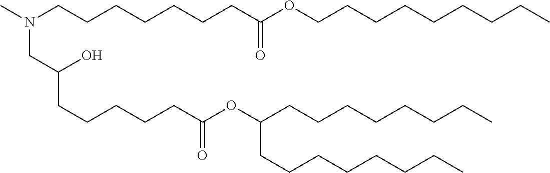 [SMILES: CCCCCCCCCOC(=O)CCCCCCCN(C)CC(O)CCCCCC(=O)OC(CCCCCCCC)CCCCCCCC] |
| I 300 | 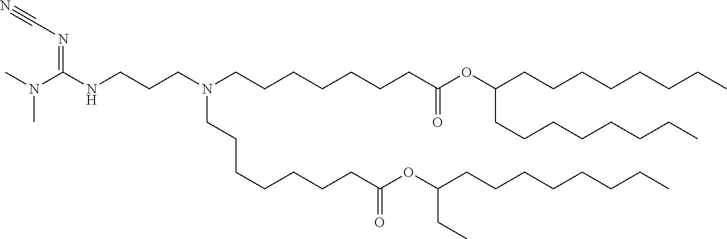 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 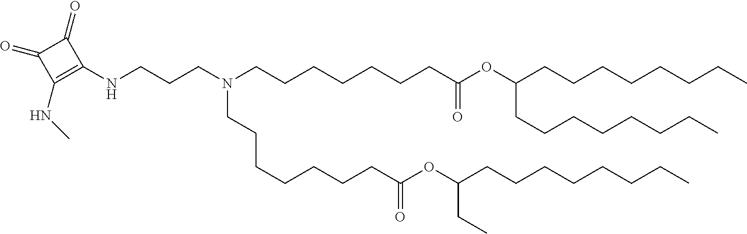 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I 302 | 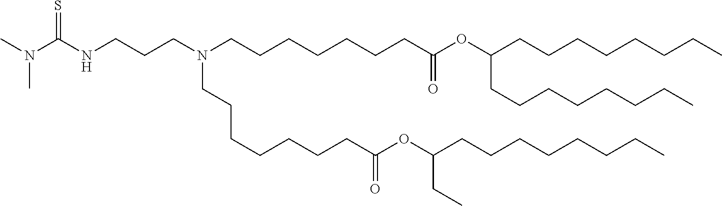 [SMILES: CCCCCCCCC(CC)OC(=O)CCCCCCCN(CCCCCCCC(=O)OC(CCCCCCCC)CCCCCCCC)CCCNC(=S)N(C)C] |
| I 303 | 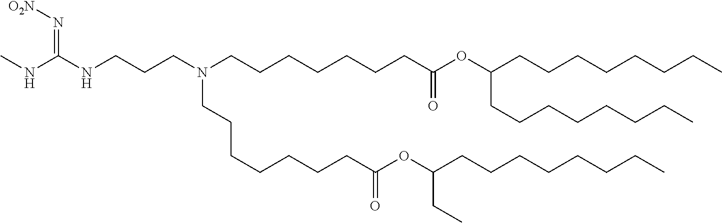 [SMILES: CCCCCCCCC(CC)OC(=O)CCCCCCCN(CCCCCCCC(=O)OC(CCCCCCCC)CCCCCCCC)CCCN/C(=N/[N+](=O)[O-])NC] |
| I 304 | 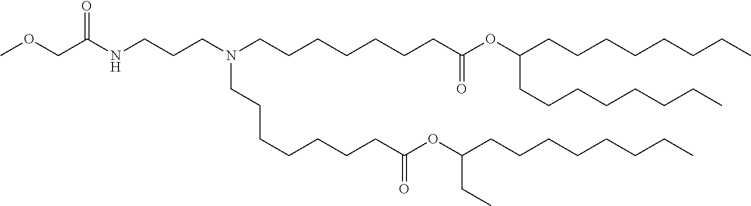 [SMILES: CCCCCCCCC(CC)OC(=O)CCCCCCCN(CCCCCCCC(=O)OC(CCCCCCCC)CCCCCCCC)CCCNC(=O)COC] |
| I 305 | 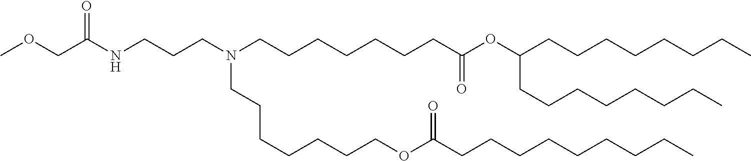 [SMILES: CCCCCCCCCC(=O)OCCCCCCCN(CCCCCCCC(=O)OC(CCCCCCCC)CCCCCCCC)CCCNC(=O)COC] |
| I 306 | 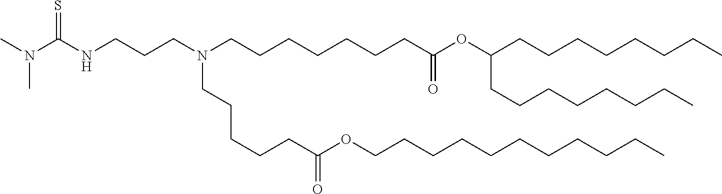 [SMILES: CCCCCCCCCCCOC(=O)CCCCCN(CCCCCCCC(=O)OC(CCCCCCCC)CCCCCCCC)CCCNC(=S)N(C)C] |
| I 307 | 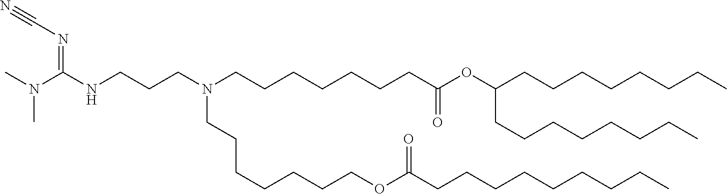 [SMILES: CCCCCCCCCC(=O)OCCCCCCCN(CCCCCCCC(=O)OC(CCCCCCCC)CCCCCCCC)CCCN/C(=N/C#N)N(C)C] |
| I 308 | 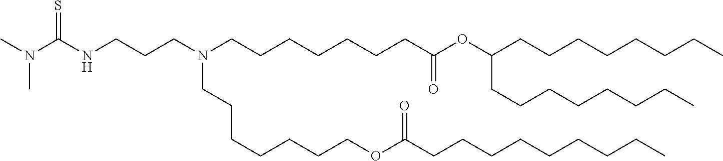 [SMILES: CCCCCCCCCC(=O)OCCCCCCCN(CCCCCCCC(=O)OC(CCCCCCCC)CCCCCCCC)CCCNC(=S)N(C)C] |
| I 309 | 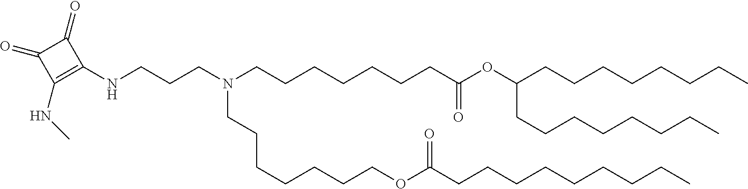 [SMILES: CCCCCCCCCC(=O)OCCCCCCCN(CCCCCCCC(=O)OC(CCCCCCCC)CCCCCCCC)CCCNc1c(NC)c(=O)c1=O] |
| I 310 | 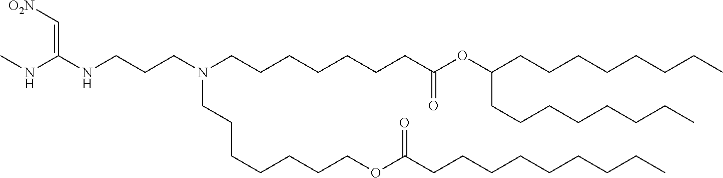 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I 311 | 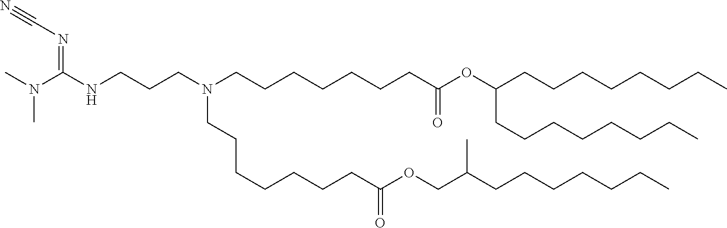 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I 312 | 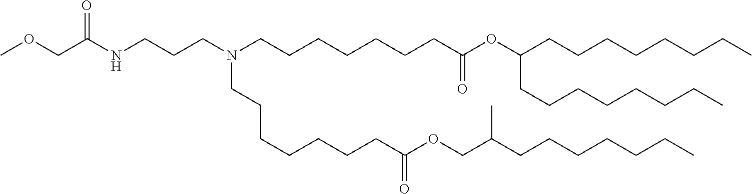 [SMILES: CCCCCCCCC(CCCCCCCC)OC(=O)CCCCCCCN(CCCCCCCC(=O)OCC(C)CCCCCCC)CCCNC(=O)COC] |
| I 313 | 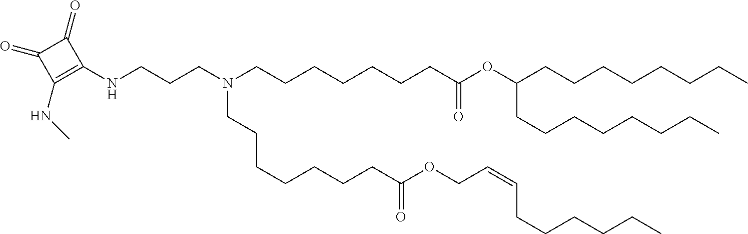 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I 314 | 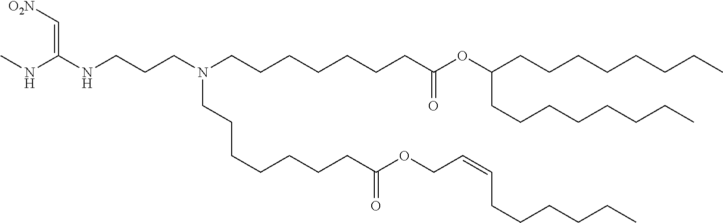 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I 315 | 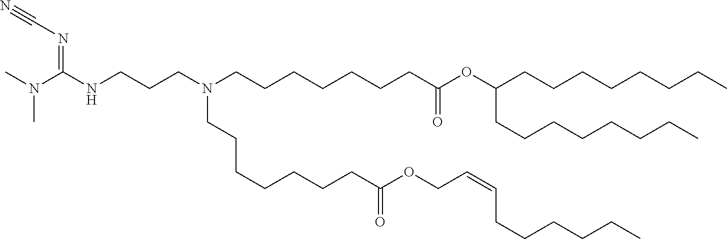 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I 316 | 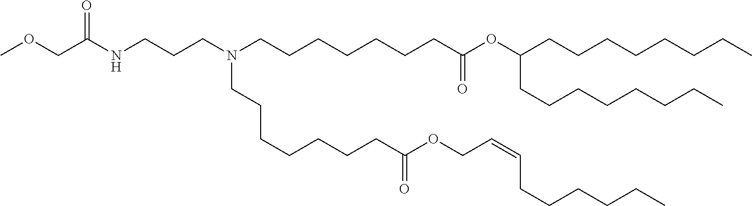 [SMILES: CCCCCC/C=C\COC(=O)CCCCCCCN(CCCCCCCC(=O)OC(CCCCCCCC)CCCCCCCC)CCCNC(=O)COC] |
| I 317 | 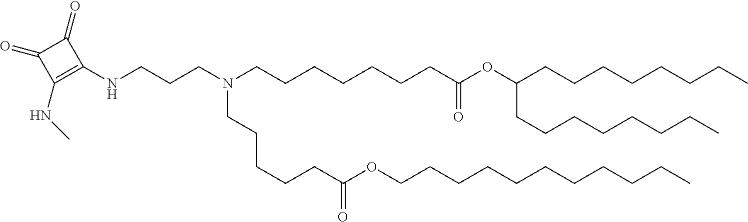 [SMILES: CCCCCCCCCCCOC(=O)CCCCCN(CCCCCCCC(=O)OC(CCCCCCCC)CCCCCCCC)CCCNc1c(NC)c(=O)c1=O] |
| I 318 | 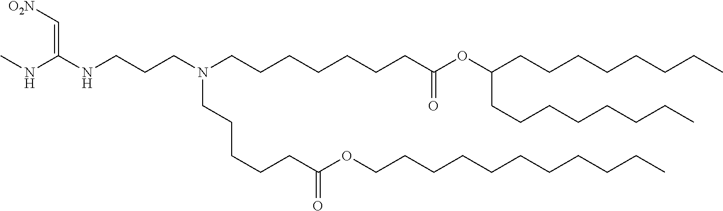 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 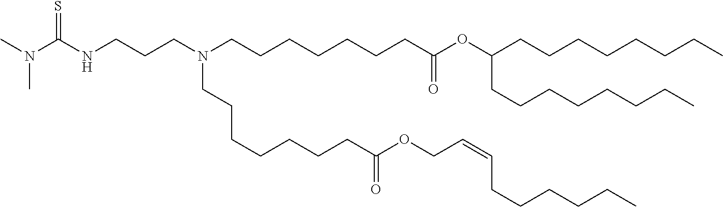 [SMILES: CCCCCC/C=C\COC(=O)CCCCCCCN(CCCCCCCC(=O)OC(CCCCCCCC)CCCCCCCC)CCCNC(=S)N(C)C] |
| I 320 | 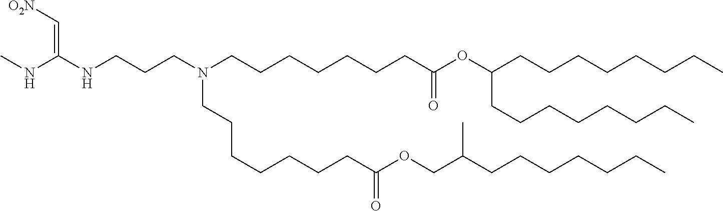 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 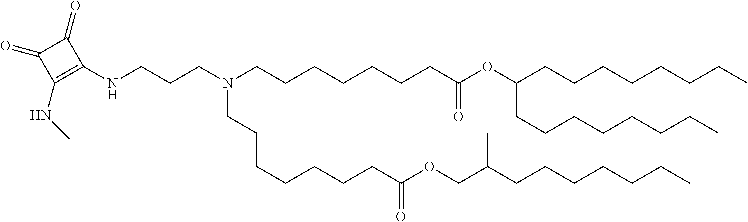 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I 322 | 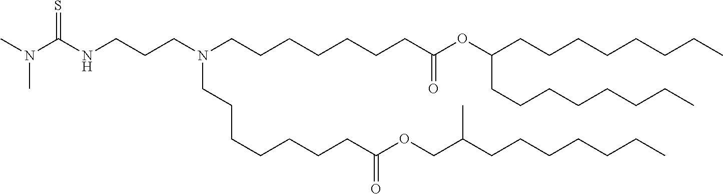 [SMILES: CCCCCCCCC(CCCCCCCC)OC(=O)CCCCCCCN(CCCCCCCC(=O)OCC(C)CCCCCCC)CCCNC(=S)N(C)C] |
| I 323 | 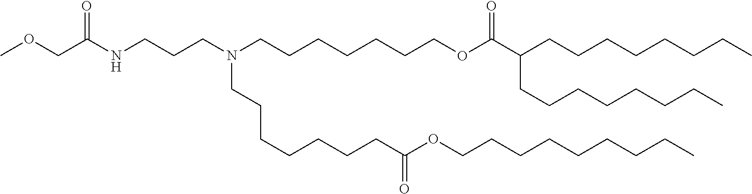 [SMILES: CCCCCCCCCOC(=O)CCCCCCCN(CCCCCCCOC(=O)C(CCCCCCCC)CCCCCCCC)CCCNC(=O)COC] |
| I 324 | 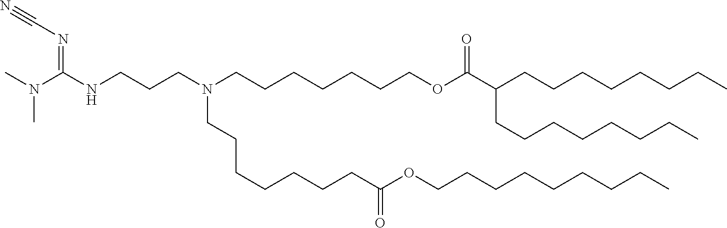 [SMILES: CCCCCCCCCOC(=O)CCCCCCCN(CCCCCCCOC(=O)C(CCCCCCCC)CCCCCCCC)CCCN/C(=N/C#N)N(C)C] |
| I 325 | 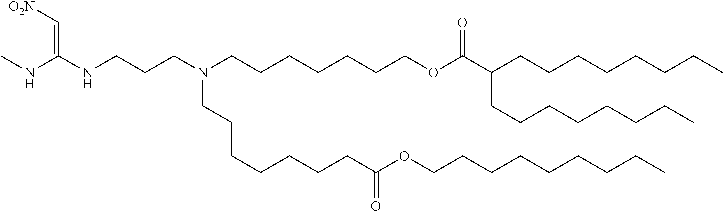 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 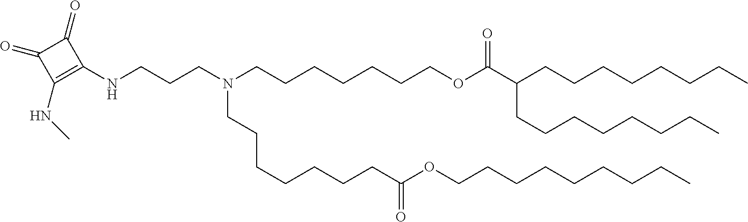 [SMILES: CCCCCCCCCOC(=O)CCCCCCCN(CCCCCCCOC(=O)C(CCCCCCCC)CCCCCCCC)CCCNc1c(NC)c(=O)c1=O] |
| I 327 | 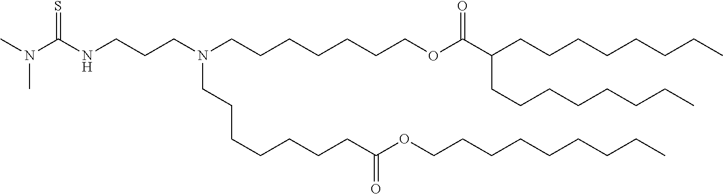 [SMILES: CCCCCCCCCOC(=O)CCCCCCCN(CCCCCCCOC(=O)C(CCCCCCCC)CCCCCCCC)CCCNC(=S)N(C)C] |
| I 328 | 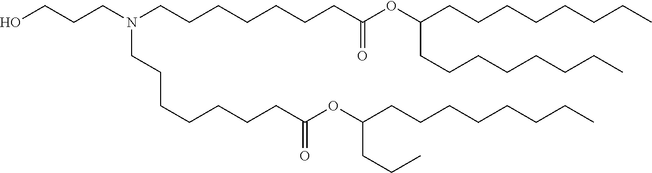 [SMILES: CCCCCCCCC(CCC)OC(=O)CCCCCCCN(CCCO)CCCCCCCC(=O)OC(CCCCCCCC)CCCCCCCC] |
| I 329 | 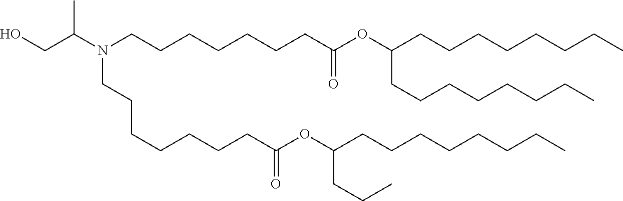 [SMILES: CCCCCCCCC(CCC)OC(=O)CCCCCCCN(CCCCCCCC(=O)OC(CCCCCCCC)CCCCCCCC)C(C)CO] |
| I 330 | 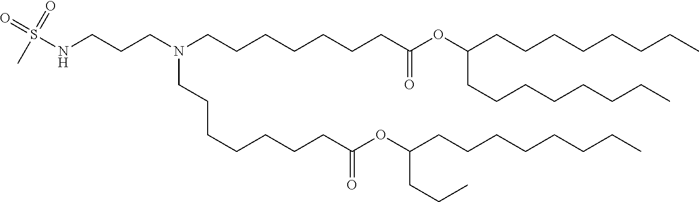 [SMILES: CCCCCCCCC(CCC)OC(=O)CCCCCCCN(CCCCCCCC(=O)OC(CCCCCCCC)CCCCCCCC)CCCNS(C)(=O)=O] |
| I 331 | 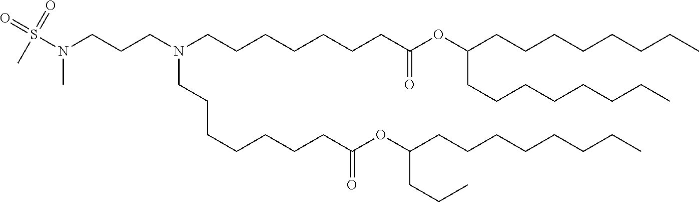 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I 332 | 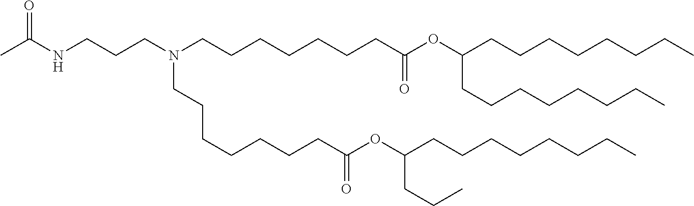 [SMILES: CCCCCCCCC(CCC)OC(=O)CCCCCCCN(CCCCCCCC(=O)OC(CCCCCCCC)CCCCCCCC)CCCNC(C)=O] |
| I 333 | 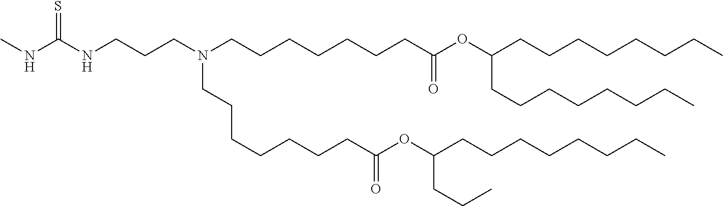 [SMILES: CCCCCCCCC(CCC)OC(=O)CCCCCCCN(CCCCCCCC(=O)OC(CCCCCCCC)CCCCCCCC)CCCNC(=S)NC] |
| I 334 | 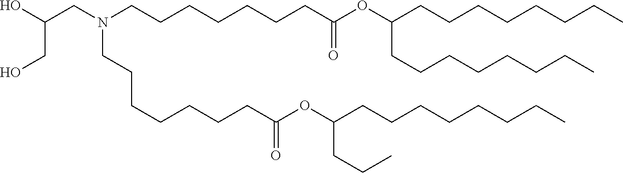 [SMILES: CCCCCCCCC(CCC)OC(=O)CCCCCCCN(CCCCCCCC(=O)OC(CCCCCCCC)CCCCCCCC)CC(O)CO] |
| I 335 | 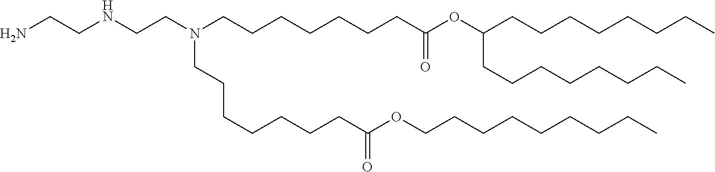 [SMILES: CCCCCCCCCOC(=O)CCCCCCCN(CCCCCCCC(=O)OC(CCCCCCCC)CCCCCCCC)CCNCCN] |
| I 336 | 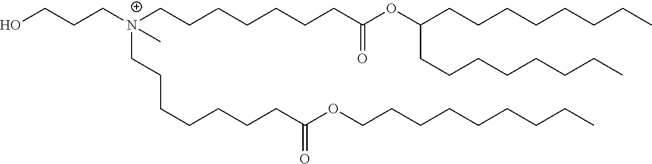 [SMILES: CCCCCCCCCOC(=O)CCCCCCC[N+](C)(CCCO)CCCCCCCC(=O)OC(CCCCCCCC)CCCCCCCC] |
| I 337 | 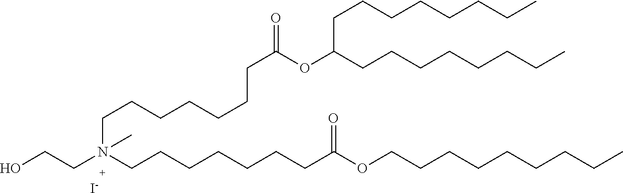 |
| I 338 | 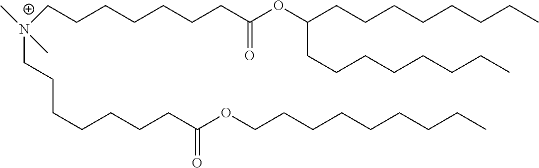 [SMILES: CCCCCCCCCOC(=O)CCCCCCC[N+](C)(C)CCCCCCCC(=O)OC(CCCCCCCC)CCCCCCCC] |
| I 339 | 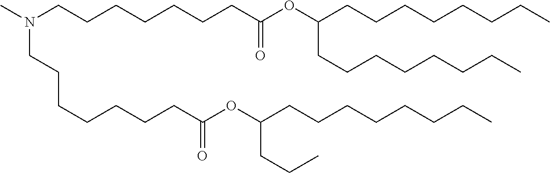 [SMILES: CCCCCCCCC(CCC)OC(=O)CCCCCCCN(C)CCCCCCCC(=O)OC(CCCCCCCC)CCCCCCCC] |
| I 340 | 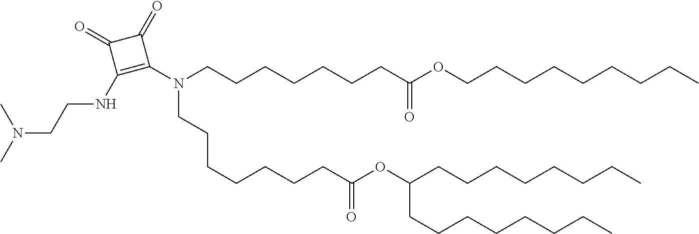 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I 341 | 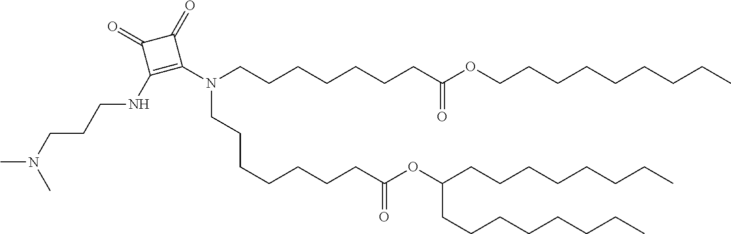 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I 342 | 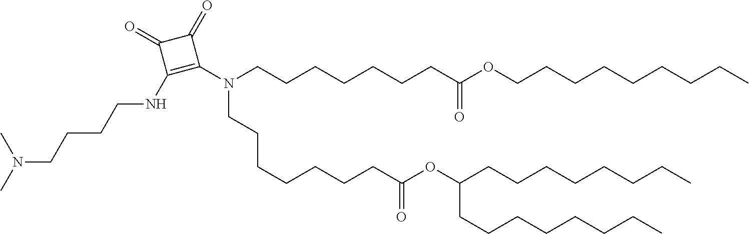 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I 343 | 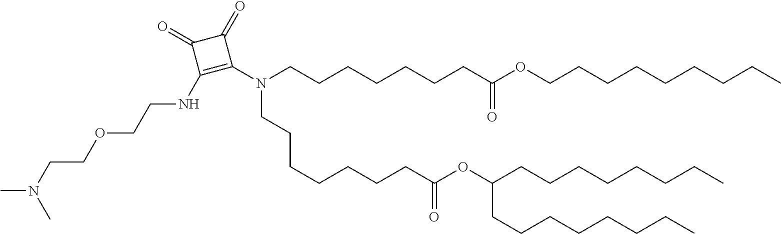 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I 344 | 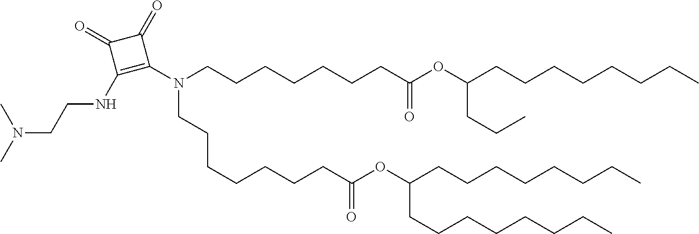 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I 345 | 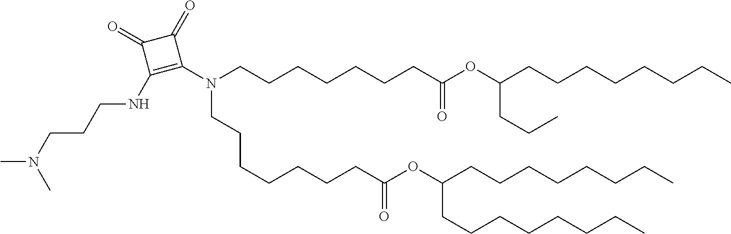 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I 346 | 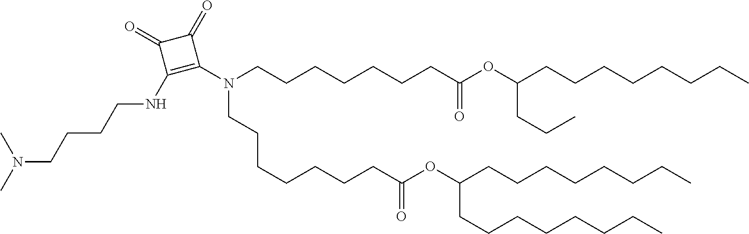 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I 347 | 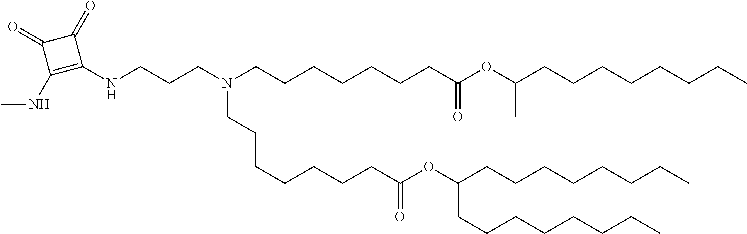 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I 348 | 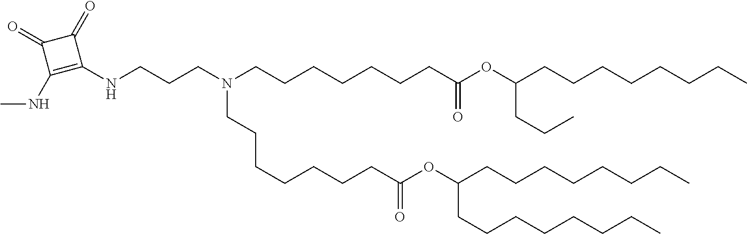 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I 349 | 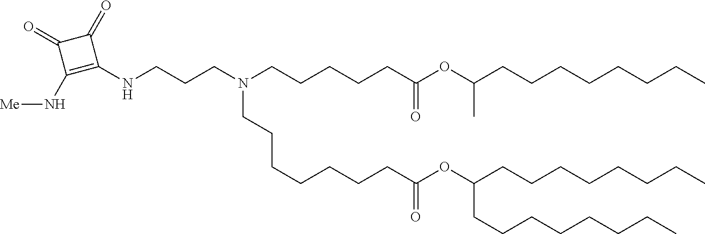 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I 350 | 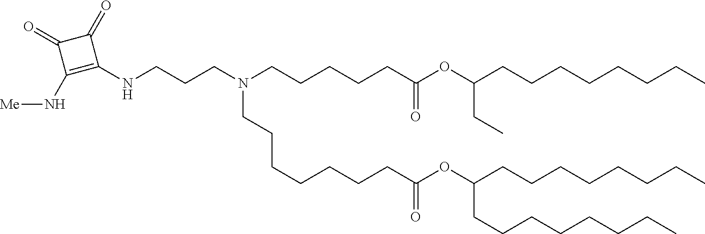 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I 351 | 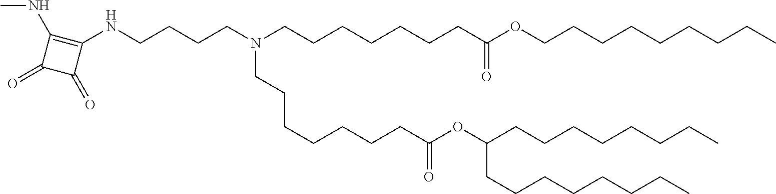 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I 352 | 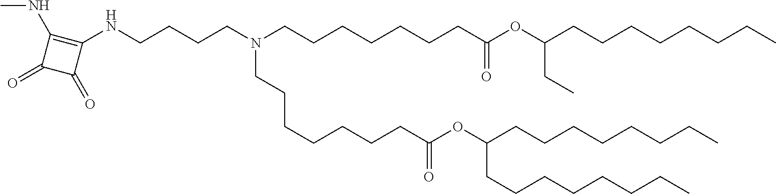 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I 353 | 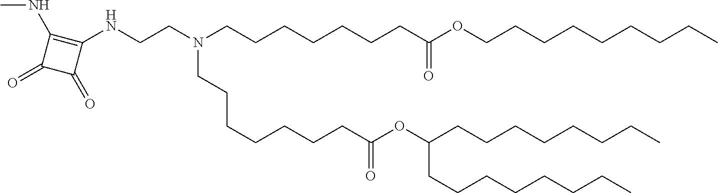 [SMILES: CCCCCCCCCOC(=O)CCCCCCCN(CCCCCCCC(=O)OC(CCCCCCCC)CCCCCCCC)CCNc1c(NC)c(=O)c1=O] |
| I 354 | 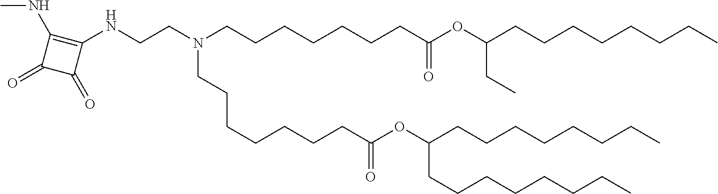 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I 355 | 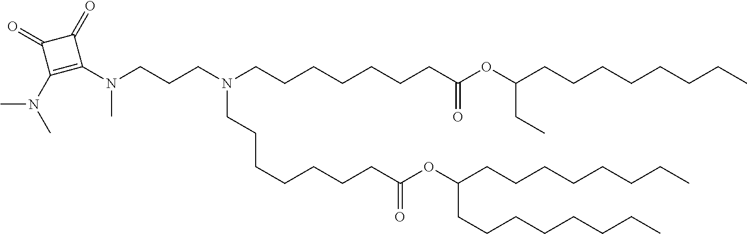 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |

In some embodiments, a lipid of the disclosure comprises Compound I-340A:

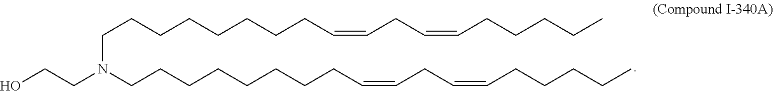 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

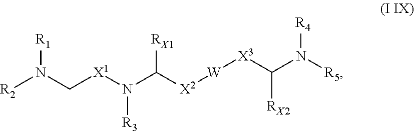

or salts or isomers thereof, wherein

W is

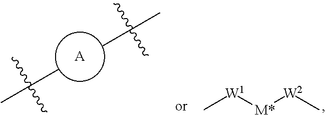

ring A is

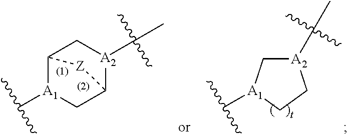

or,

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R⁶ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

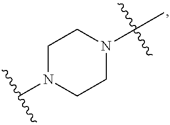 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

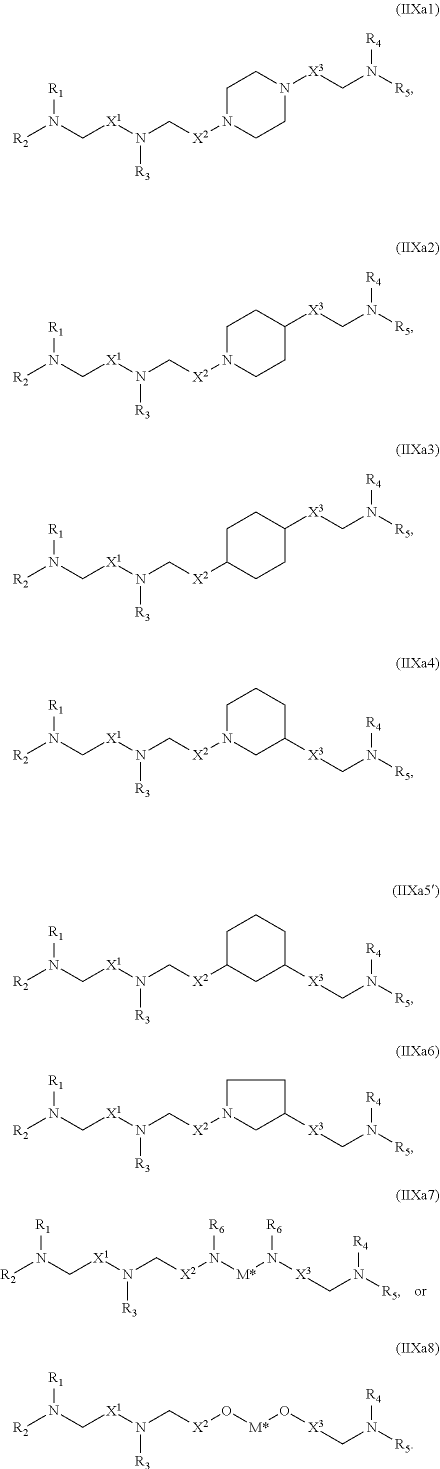

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

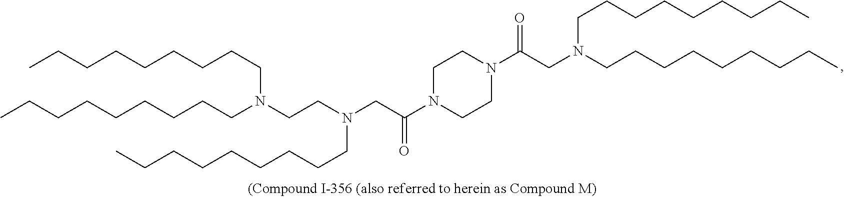 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.
In some embodiments, the ionizable lipid is

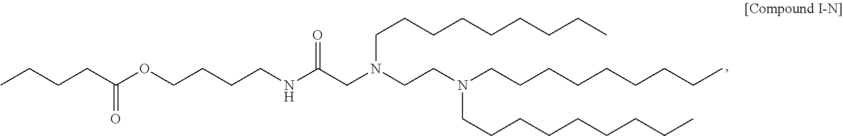 [SMILES: CCCCCCCCCN(CCCCCCCCC)CCN(CCCCCCCCC)CC(=O)NCCCCOC(=O)CCCC]

or a salt thereof.
In some embodiments, the ionizable lipid is

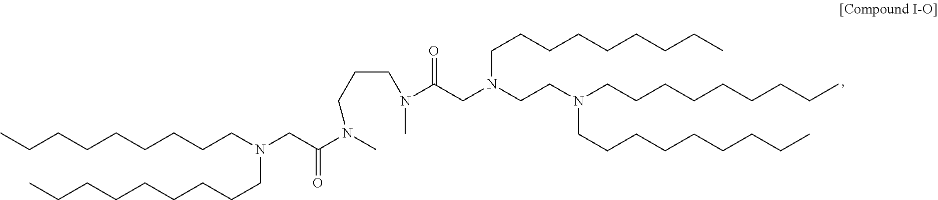 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.
In some embodiments, the ionizable lipid is

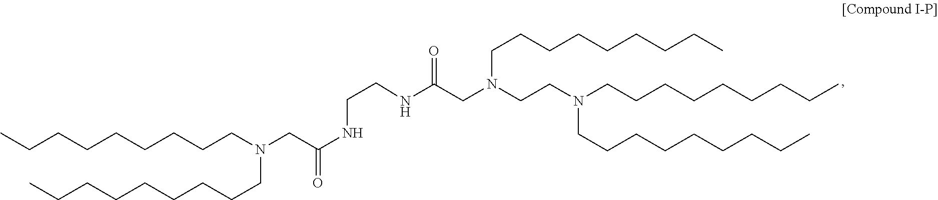 [SMILES: CCCCCCCCCN(CCCCCCCCC)CCN(CCCCCCCCC)CC(=O)NCCNC(=O)CN(CCCCCCCCC)CCCCCCCCC]

or a salt thereof.
In some embodiments, the ionizable lipid is

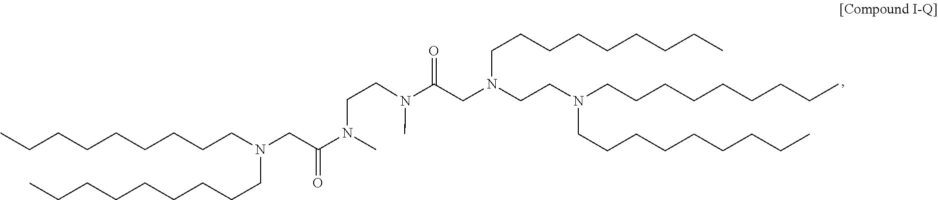 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (I IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8)) (each of these preceeded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) is about 45 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) is about 40 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceeded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)-N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety. For example, the ionizable amino lipids include, but not limited to:

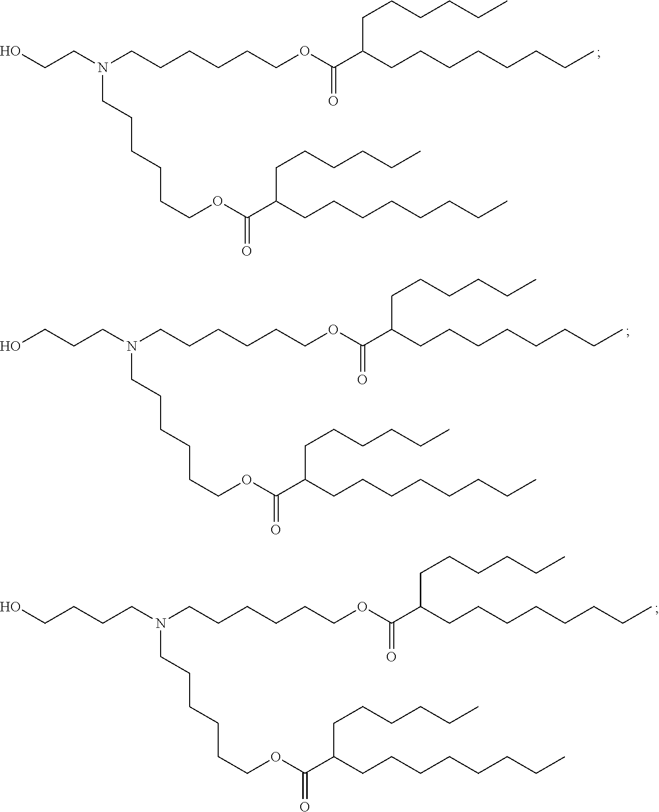 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids of the invention can also be the compounds disclosed in International Publication No. WO 2015/199952 A1, hereby incorporated by reference in its entirety. For example, the ionizable amino lipids include, but not limited to:

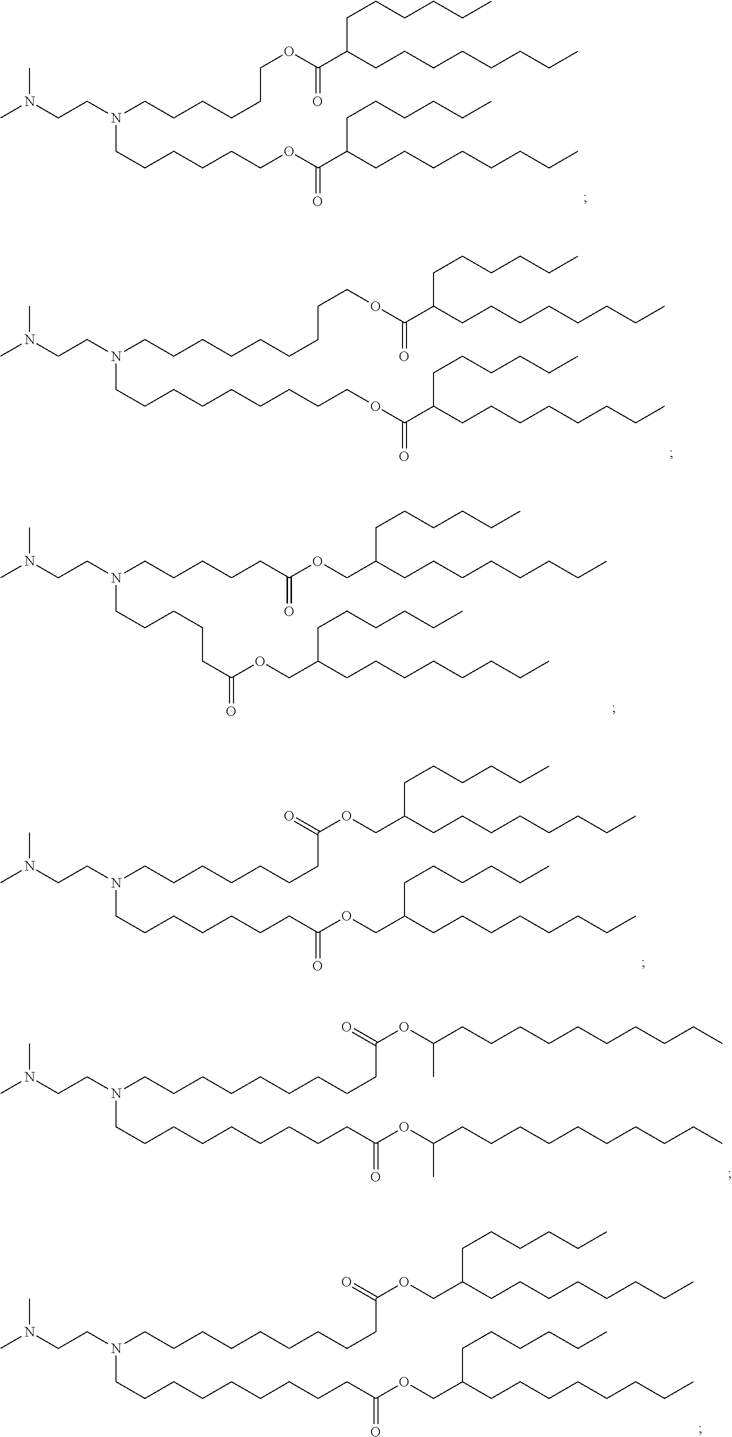

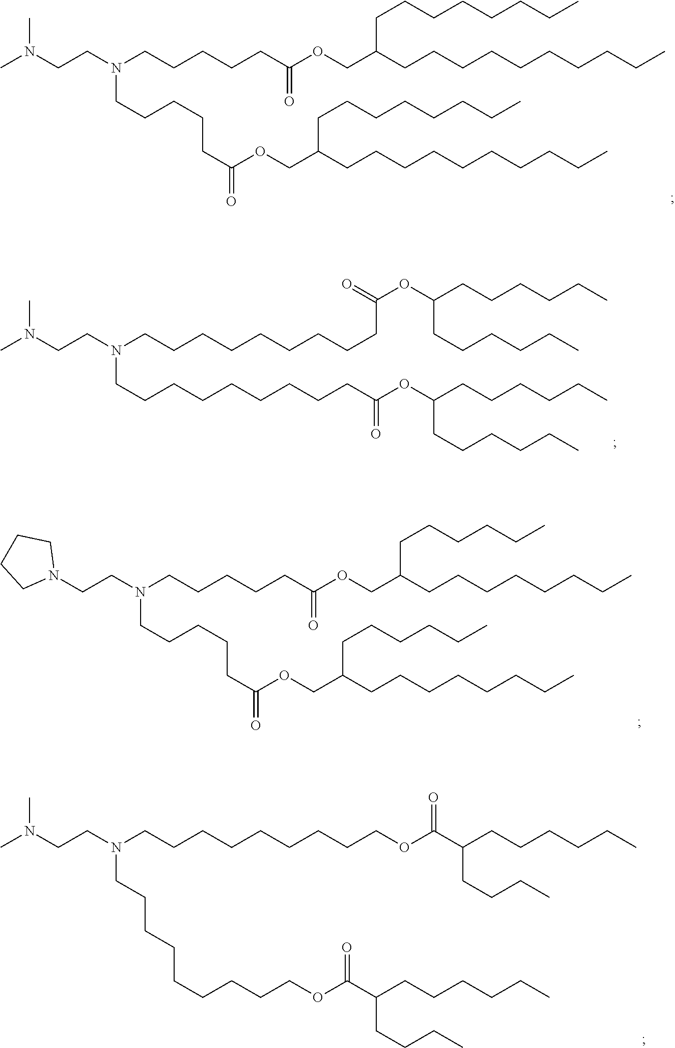

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceeded by the letter I for clarity).

In any of the foregoing or related aspects, the ionizable lipid of the LNP of the disclosure comprises a compound comprising any of Compound Nos. I 1-356.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In any of the foregoing or related aspects, the synthesis of compounds of the invention, e.g. compounds comprising any of Compound Nos. 1-356, follows the synthetic descriptions in U.S. Provisional Patent Application No. 62/733,315, filed Sep. 19, 2018.

### Representative Synthetic Routes

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

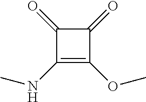

### Chemical Formula: C₆H₇NO₃

### Molecular Weight: 141.13

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

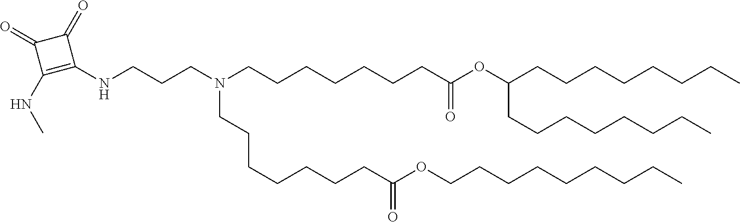 [SMILES: CCCCCCCCCOC(=O)CCCCCCCN(CCCCCCCC(=O)OC(CCCCCCCC)CCCCCCCC)CCCNc1c(NC)c(=O)c1=O]

### Chemical Formula: C₅₀H₉₃N₃O₆

### Molecular Weight: 832.31

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

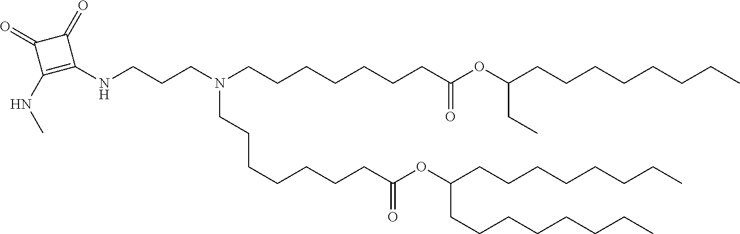 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Chemical Formula: C₅₂H₉₇N₃O₆

### Molecular Weight: 860.36

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

(ii) Cholesterol/Structural Lipids

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

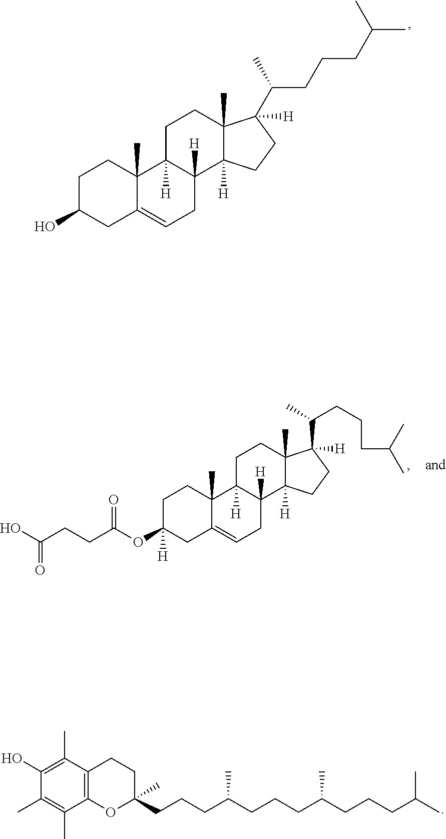

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

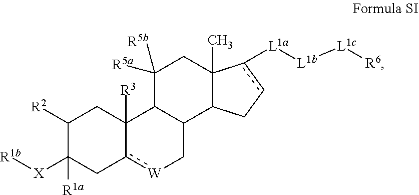

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

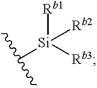

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

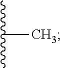

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

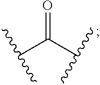 [SMILES: *C(*)=O]

L^{1a} is absent,

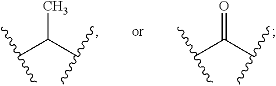

L^{1b} is absent,

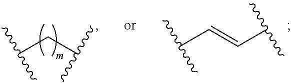

m is 1, 2, or 3;

L^{1c} is absent,

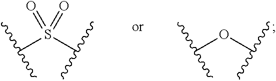

and

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl,

or a pharmaceutically acceptable salt thereof.

In some embodiments, the compound has the structure of Formula SIa:

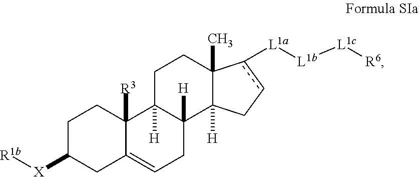

or a pharmaceutically acceptable salt thereof.

In some embodiments, the compound has the structure of Formula SIb:

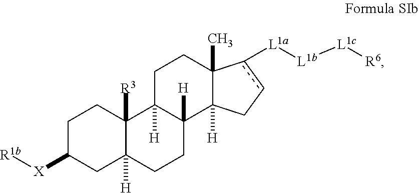

or a pharmaceutically acceptable salt thereof.

In some embodiments, the compound has the structure of Formula SIc:

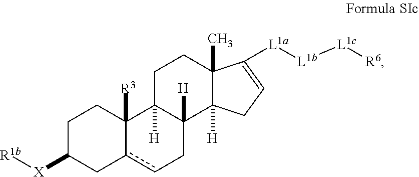

or a pharmaceutically acceptable salt thereof.

In some embodiments, the compound has the structure of Formula SId:

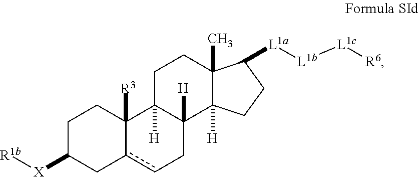

or a pharmaceutically acceptable salt thereof.

In some embodiments, L^{1a} is absent. In some embodiments, L^{1a} is

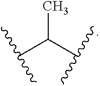 [SMILES: *C(*)C]

In some embodiments, L^{1a} is

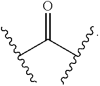 [SMILES: *C(*)=O]

In some embodiments, L^{1b} is absent. In some embodiments, L^{1b} is

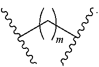

In some embodiments, L^{1b} is

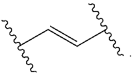 [SMILES: */C=C/*]

In some embodiments, m is 1 or 2. In some embodiments, m is 1. In some embodiments, m is 2.

In some embodiments, L^{1c} is absent. In some embodiments, L^{1c} is

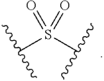 [SMILES: *S(*)(=O)=O]

In some embodiments, L^{1c} is

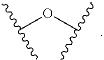 [SMILES: *O*]

In some embodiments, R⁶ is optionally substituted C₆-C₁₀ aryl.

In some embodiments, R⁶ is

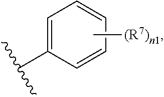

where

n1 is 0, 1, 2, 3, 4, or 5; and

each R⁷ is, independently, halo or optionally substituted C₁-C₆ alkyl.

In some embodiments, each R⁷ is, independently,

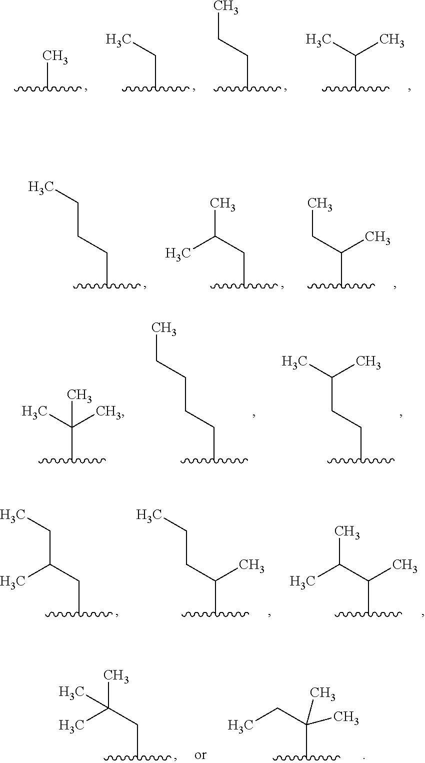

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

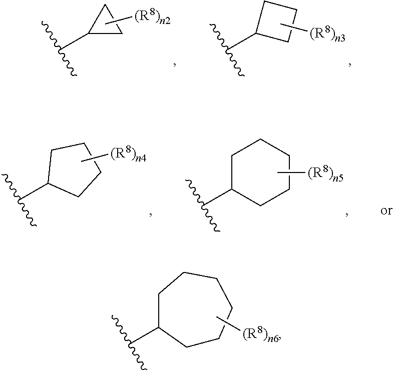

where

n2 is 0, 1, 2, 3, 4, or 5;

n3 is 0, 1, 2, 3, 4, 5, 6, or 7;

n4 is 0, 1, 2, 3, 4, 5, 6, 7, 8, or 9;

n5 is 0, 1, 2, 3, 4, 5, 6, 7, 8, 9, 10, or 11;

n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently,

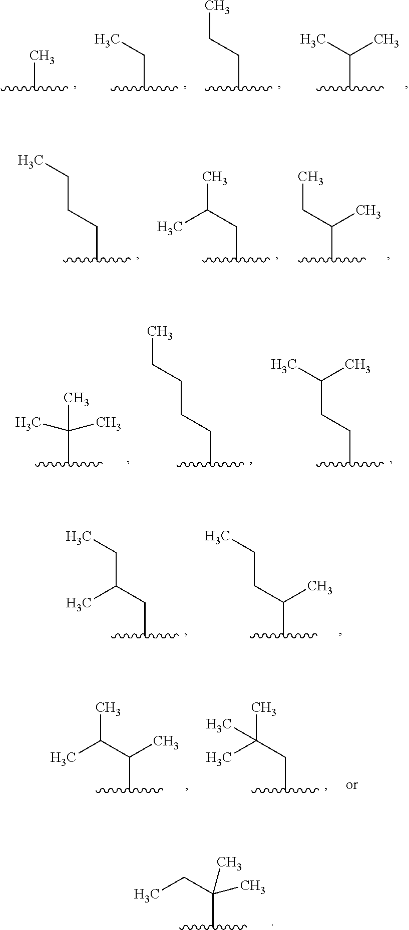

In some embodiments, R⁶ is optionally substituted C₃-C₁₀ polycycloalkyl.

In some embodiments, R⁶ is

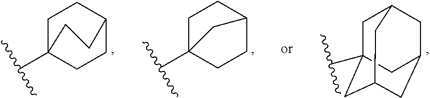 [SMILES: *C12CCC(CC1)CC2 ; *C12CCC(CC1)C2 ; *C12CC3CC(CC(C3)C1)C2]

In some embodiments, R⁶ is optionally substituted C₃-C₁₀ cycloalkenyl.

In some embodiments, R⁶ is

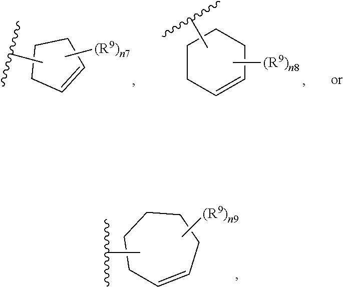

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

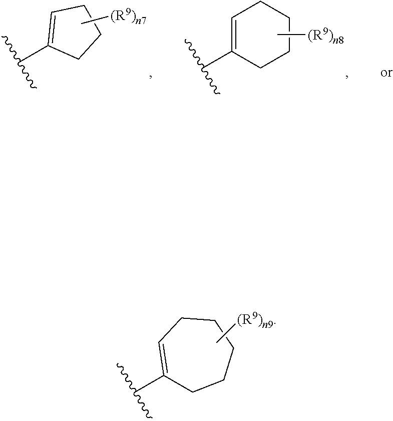

In some embodiments, each R⁹ is, independently,

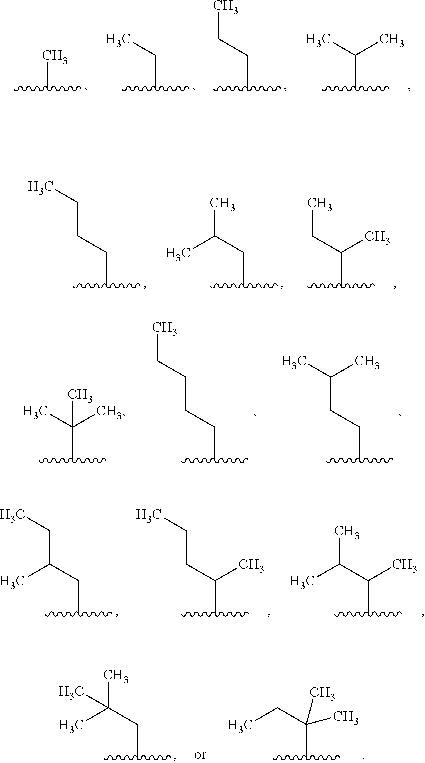

In some embodiments, R⁶ is optionally substituted C₂-C₉ heterocyclyl.

In some embodiments, R⁶ is

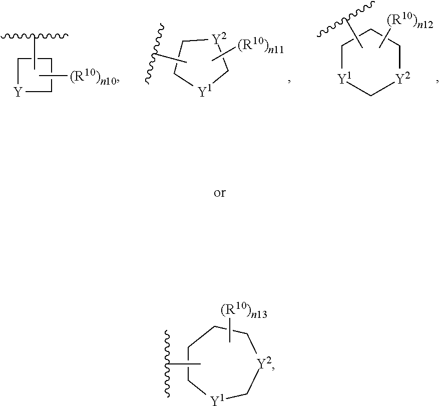

where

n10 is 0, 1, 2, 3, 4, or 5;

n11 is 0, 1, 2, 3, 4, or 5;

n12 is 0, 1, 2, 3, 4, 5, 6, or 7;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;

each of R^{11a} and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O.

In some embodiments, Y² is O. In some embodiments, Y² is CR^{11a}R^{11b}.

In some embodiments, each R¹⁰ is, independently,

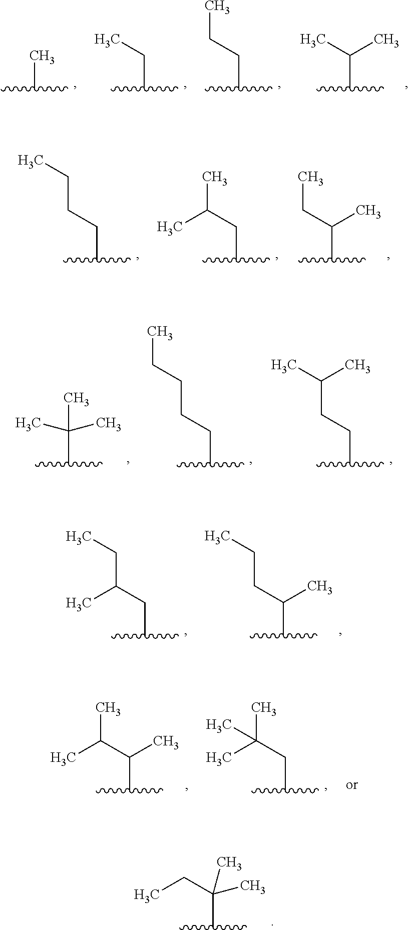

In some embodiments, R⁶ is optionally substituted C₂-C₉ heteroaryl.

In some embodiments, R⁶ is

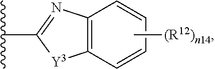

where

Y³ is NR^{C}, O, or S

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, R⁶ is

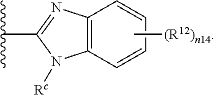

In some embodiments, R⁶ is

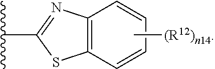

In an aspect, the structural lipid of the invention features a compound having the structure of Formula SII:

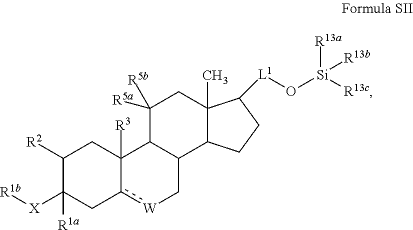

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

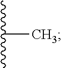

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

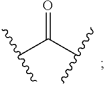 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula SIIa:

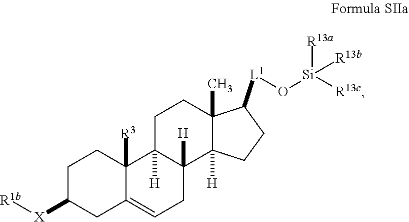

or a pharmaceutically acceptable salt thereof.

In some embodiments, the compound has the structure of Formula SIIb:

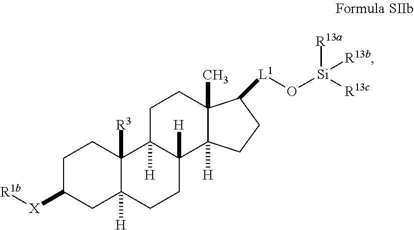

or a pharmaceutically acceptable salt thereof.

In some embodiments, L¹ is

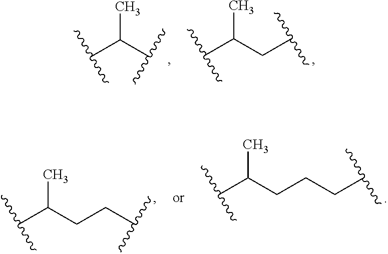

In some embodiments, each of R^{13a}, R^{13b}, and R^{13c} is, independently,

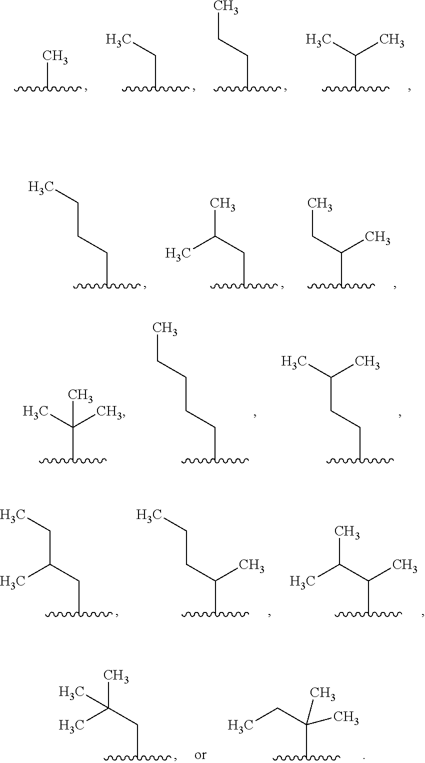

In an aspect, the structural lipid of the invention features a compound having the structure of Formula SIII:

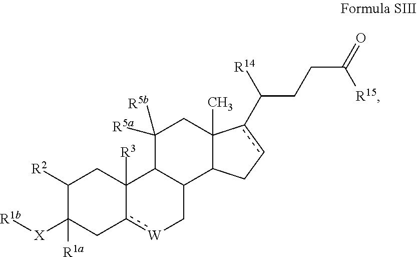

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

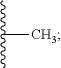

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

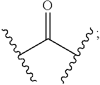 [SMILES: *C(*)=O]

R¹⁴ is H or C₁-C₆ alkyl; and

R¹⁵ is

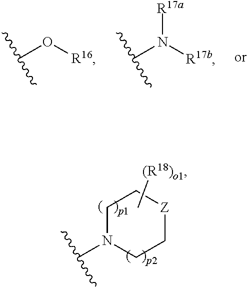

where

R¹⁶ is H or optionally substituted C₁-C₆ alkyl;

R^{17b} is H, OR^{17c}, optionally substituted C₆-C₁₀ aryl, or optionally substituted C₁-C₆ alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂O, S, or NR^{D}, where RD is H or optionally substituted C₁-C₆ alkyl; and

each R¹⁸ is, independently, halo or optionally substituted C₁-C₆ alkyl, or a pharmaceutically acceptable salt thereof.

In some embodiments, the compound has the structure of Formula SIIIa:

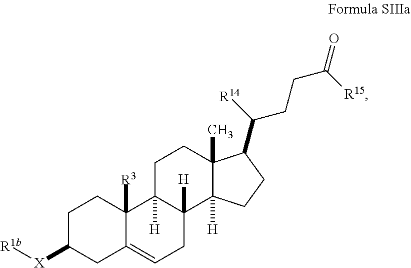

or a pharmaceutically acceptable salt thereof.

In some embodiments, the compound has the structure of Formula SIIIb:

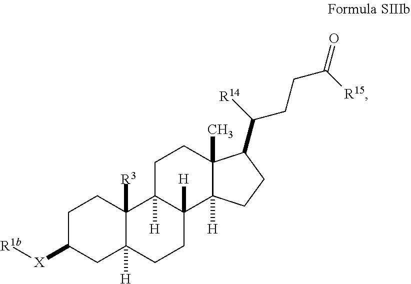

or a pharmaceutically acceptable salt thereof.

In some embodiments, R¹⁴ is H,

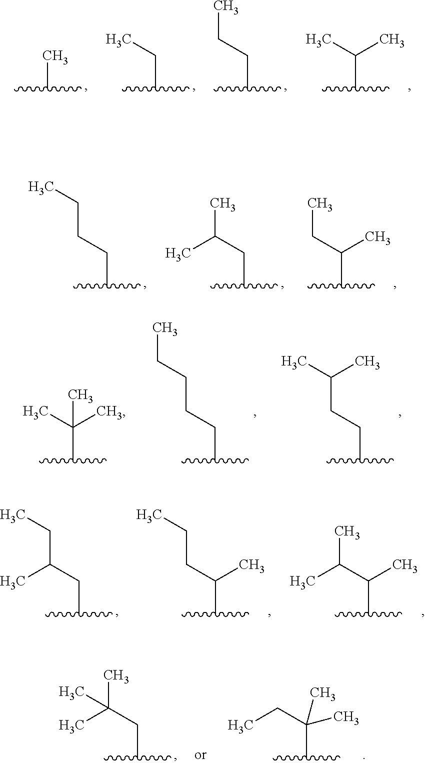

In some embodiments, R¹⁴ is

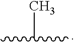

In some embodiments, R″ is

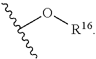

In some embodiments, R¹⁵ is

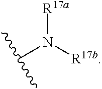

In some embodiments, R¹⁶ is H. In some embodiments, R¹⁶ is

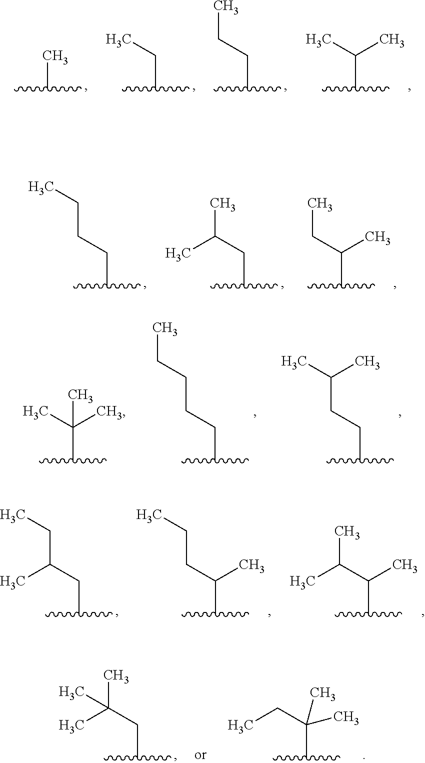

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

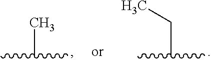

In some embodiments, R^{17c} is H. In some embodiments, R¹⁷ is

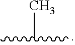

In some embodiments, R¹⁵ is

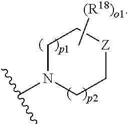

In some embodiments, each R¹⁸ is, independently,

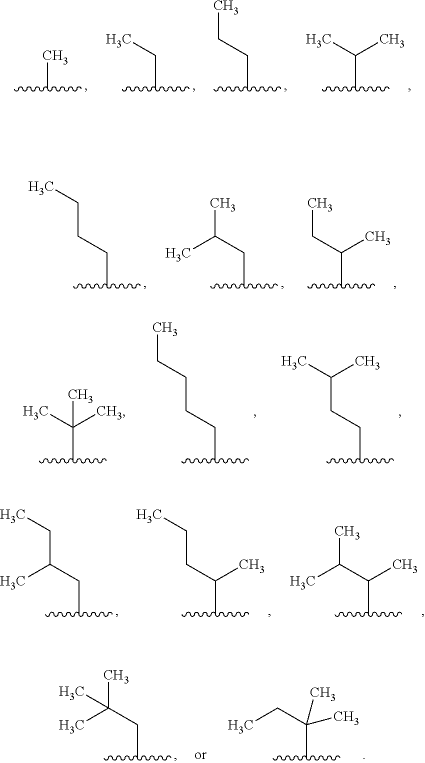

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

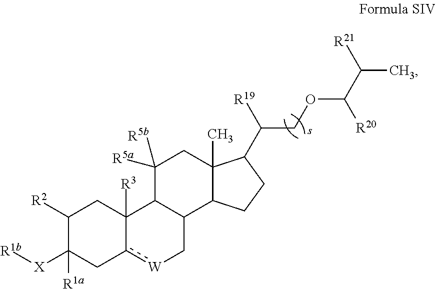

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

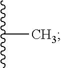

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

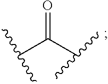 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

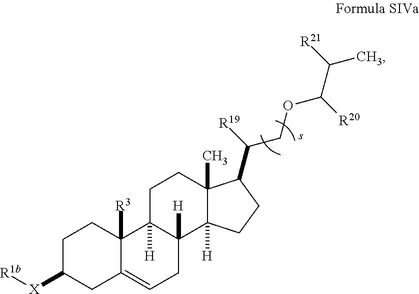

or a pharmaceutically acceptable salt thereof.

In some embodiments, the compound has the structure of Formula SIVb:

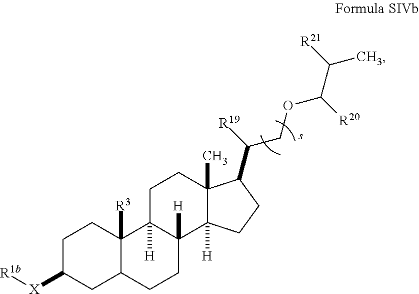

or a pharmaceutically acceptable salt thereof.

In some embodiments, R¹⁹ is H,

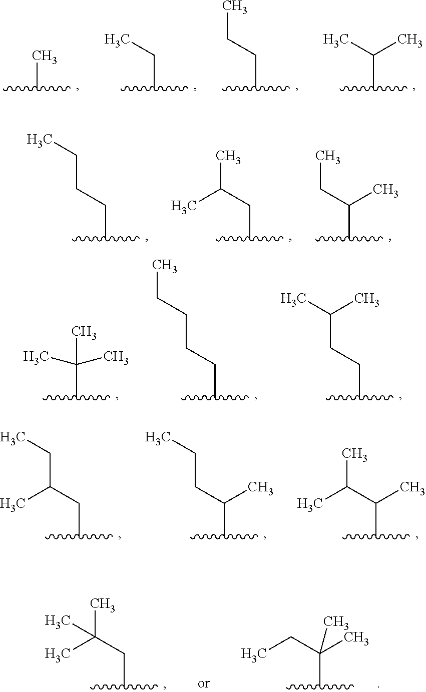

In some embodiments, R¹⁹ is

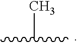

In some embodiments, R²⁰ is,

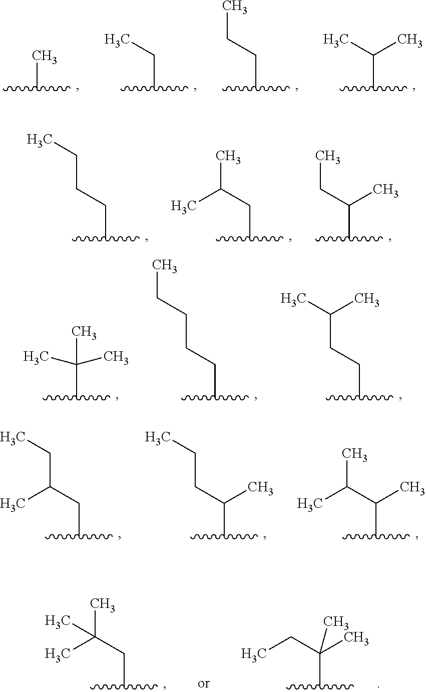

In some embodiments, R²¹ is H,

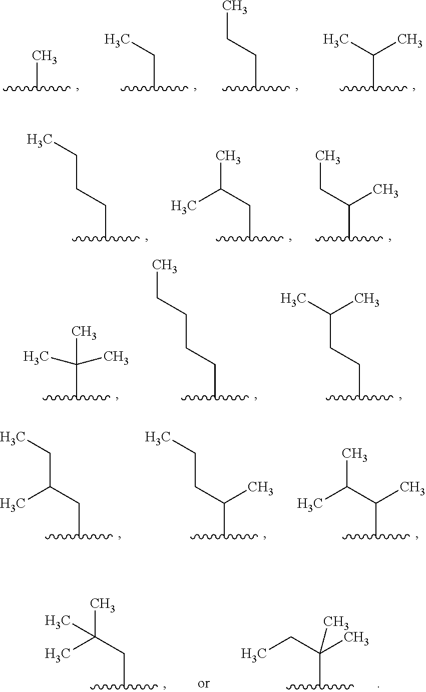

In an aspect, the structural lipid of the invention features, a compound having the structure of Formula SV:

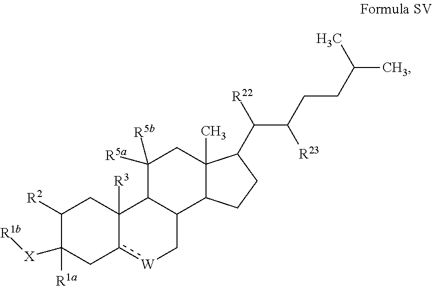

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

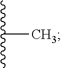

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

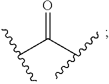 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

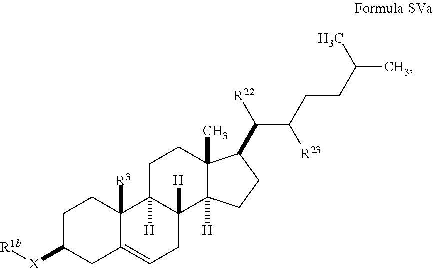

or a pharmaceutically acceptable salt thereof.

In some embodiments, the compound has the structure of Formula SVb:

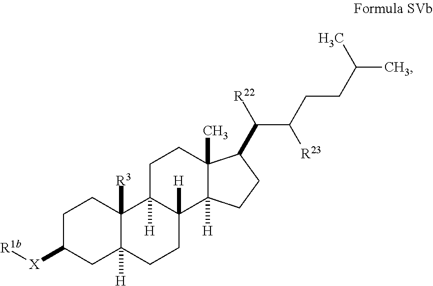

or a pharmaceutically acceptable salt thereof.

In some embodiments, R²² is H,

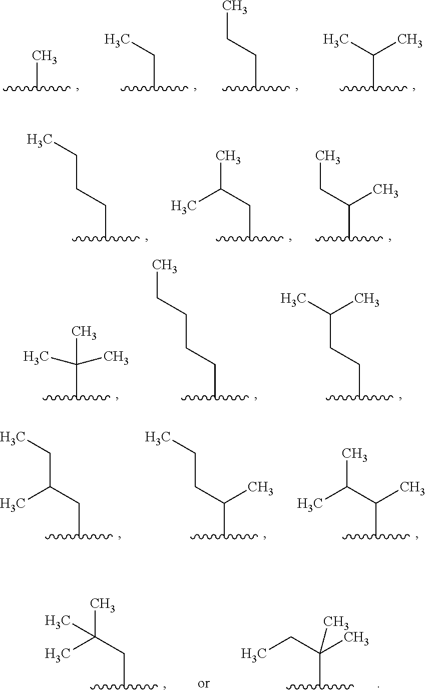

In some embodiments, R²² is

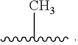

In some embodiments, R²³ is

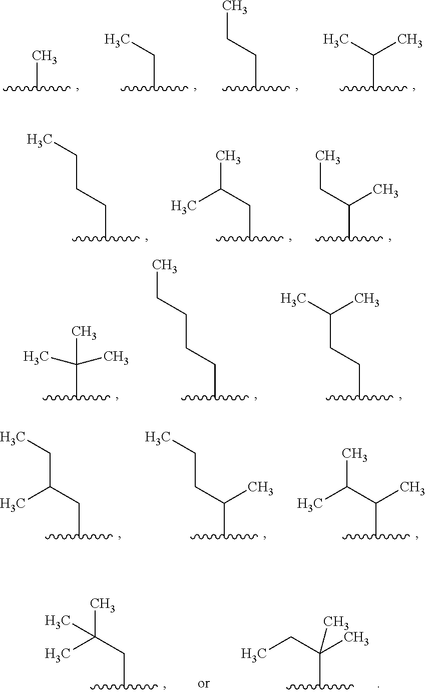

In an aspect, the structural lipid of the invention features a compound having the structure of Formula SVI:

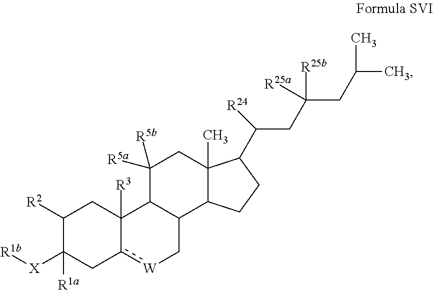

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A} where R^{A} is H or optionally substituted C₁-C₆ alkyl;

R³ is H or

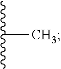

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

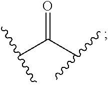 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula SVIa:

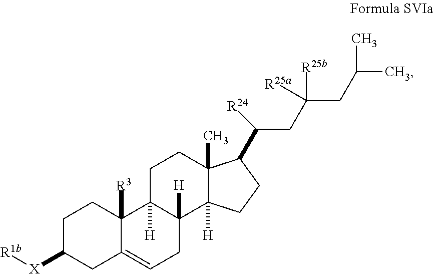

or a pharmaceutically acceptable salt thereof.

In some embodiments, the compound has the structure of Formula SVIb:

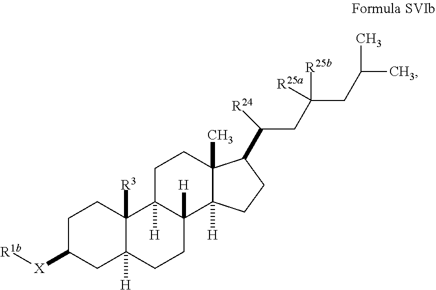

or a pharmaceutically acceptable salt thereof.

In some embodiments, R²⁴ is H,

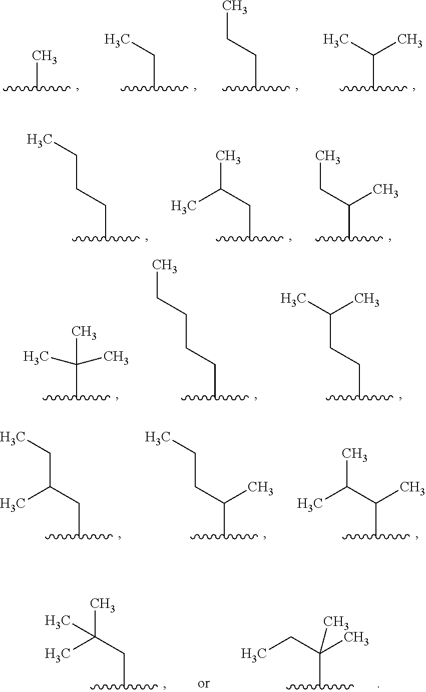

In some embodiments, R²⁴ is

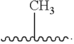

In some embodiments, each of R^{25a} and R^{25b} is, independently,

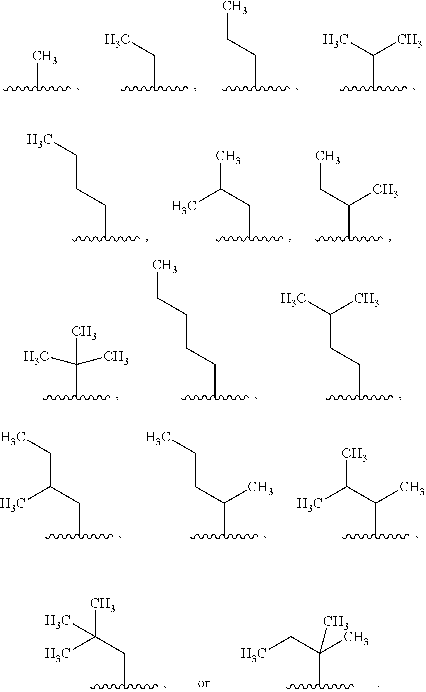

In an aspect, the structural lipid of the invention features a compound having the structure of Formula SVII:

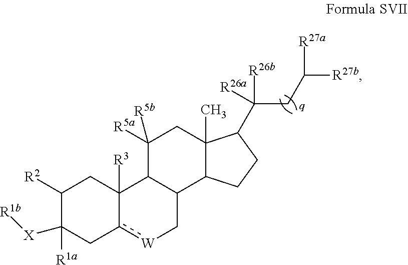

where

R^{1a} is H, optionally substituted C₁-C₆ alkyl, optionally substituted C₂-C₆ alkenyl, optionally substituted C₂-C₆ alkynyl, or

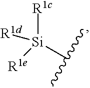

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is H or

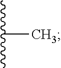

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

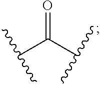 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

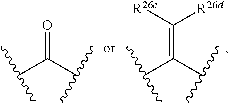

where each of R^{26c} and R²⁶ is, independently, H or optionally substituted C₁-C₆ alkyl; and

each of R^{27a} and R^{27b} is H, hydroxyl, or optionally substituted C₁-C₆ alkyl, or a pharmaceutically acceptable salt thereof.

In some embodiments, the compound has the structure of Formula SVIIa:

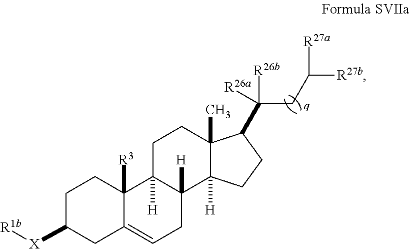

or a pharmaceutically acceptable salt thereof.

In some embodiments, the compound has the structure of Formula SVIIb:

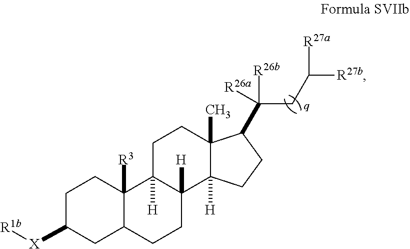

or a pharmaceutically acceptable salt thereof.

In some embodiments, R^{26a} and R^{26b} is, independently, H,

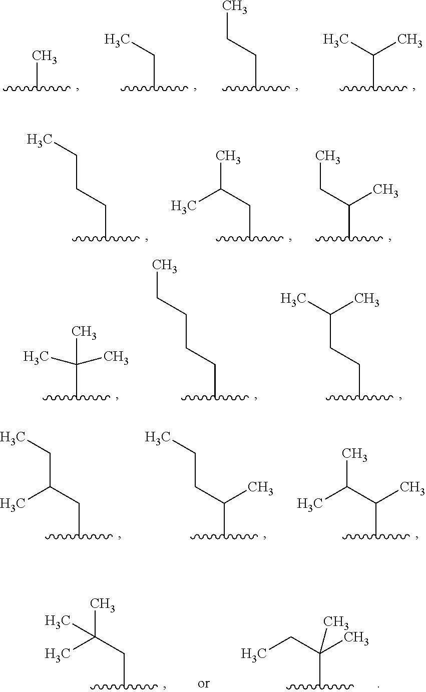

In some embodiments, R^{26a} and R^{26b}, together with the atom to which each is attached, combine to form

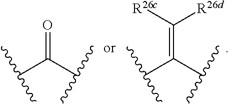

In some embodiments, R^{26a} and R^{26b}, together with the atom to which each is attached, combine to form

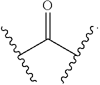 [SMILES: *C(*)=O]

In some embodiments, R^{26a} and R^{26b}, together with the atom to which each is attached, combine to form

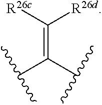

In some embodiments, where each of R^{26c} and R²⁶ is, independently, H,

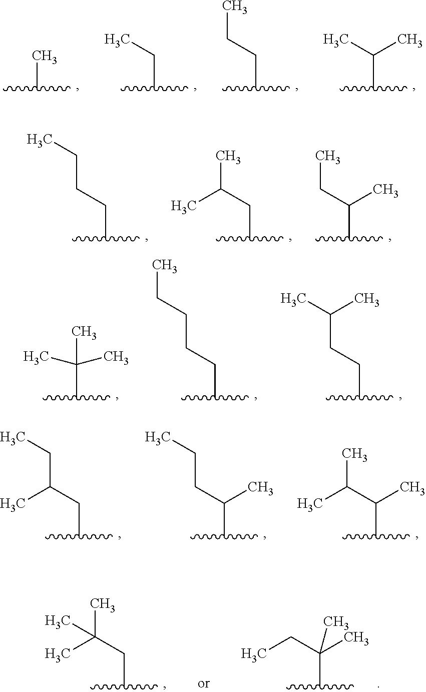

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

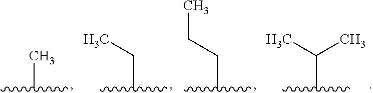

In an aspect, the structural lipid of the invention features a compound having the structure of Formula SVIII:

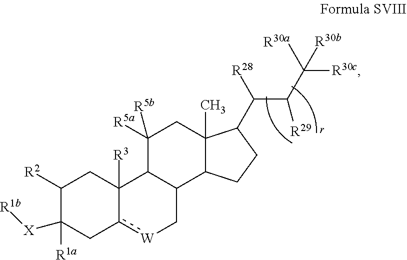

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

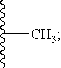

[symbol] a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

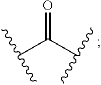 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl,

or a pharmaceutically acceptable salt thereof.

In some embodiments, the compound has the structure of Formula SVIIIa:

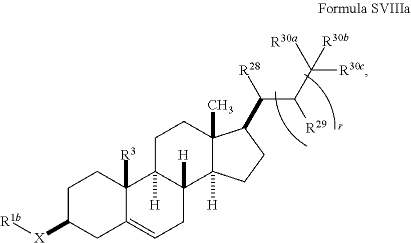

or a pharmaceutically acceptable salt thereof.

In some embodiments, the compound has the structure of Formula SVIIIb:

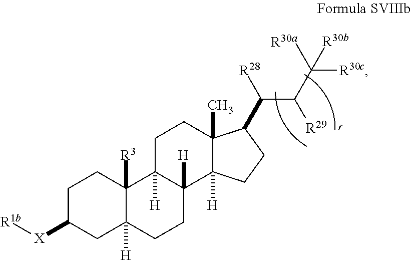

or a pharmaceutically acceptable salt thereof.

In some embodiments, R²⁸ is H,

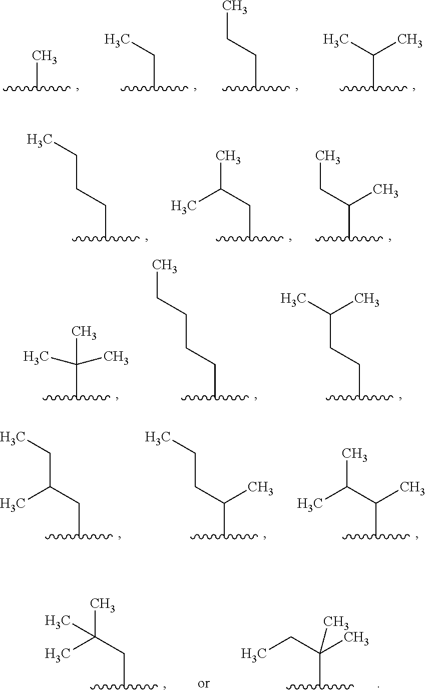

In some embodiments, R²⁸ is

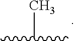

In some embodiments, each of R^{30a}, R^{30b}, and R^{30c} is, independently,

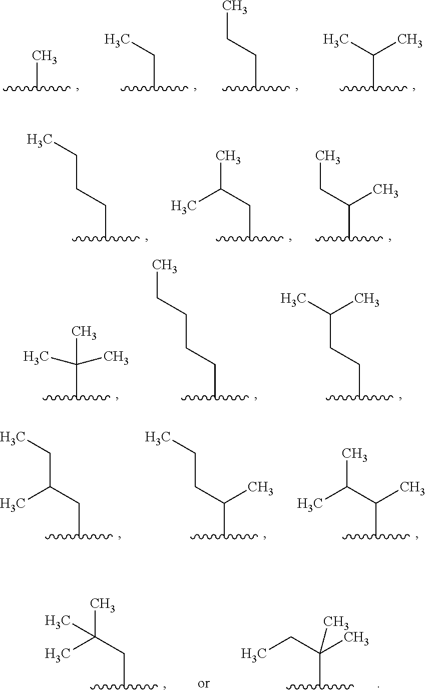

In some embodiments, r is 1. In some embodiments, r is 2. In some embodiments, r is 3.

In some embodiments, each R²⁹ is, independently, H,

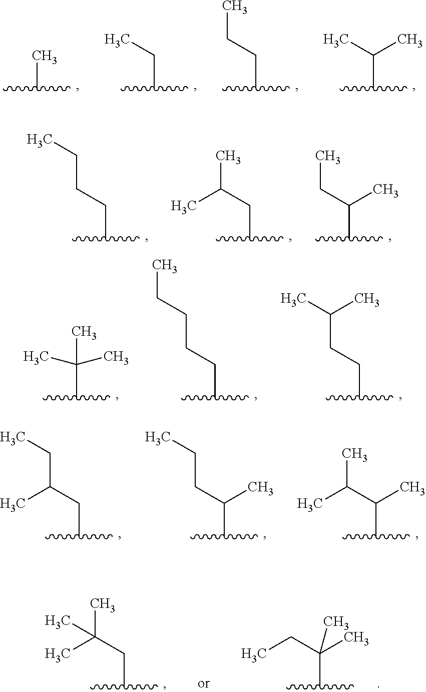

In some embodiments, each R²⁹ is, independently, H or

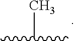

In an aspect, the structural lipid of the invention features a compound having the structure of Formula SIX:

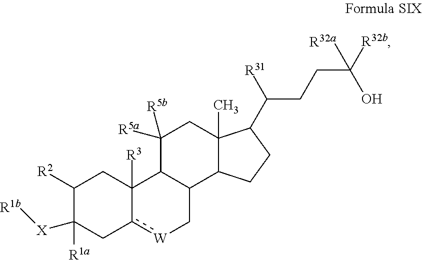

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

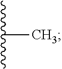

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

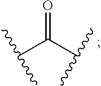 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

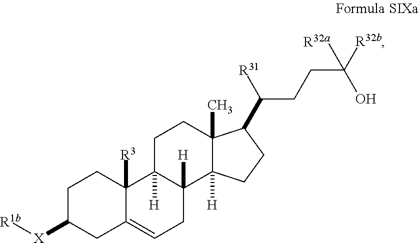

or a pharmaceutically acceptable salt thereof.

In some embodiments, the compound has the structure of Formula SIXb:

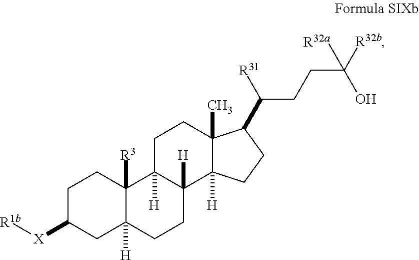

or a pharmaceutically acceptable salt thereof.

In some embodiments, R³¹ is H,

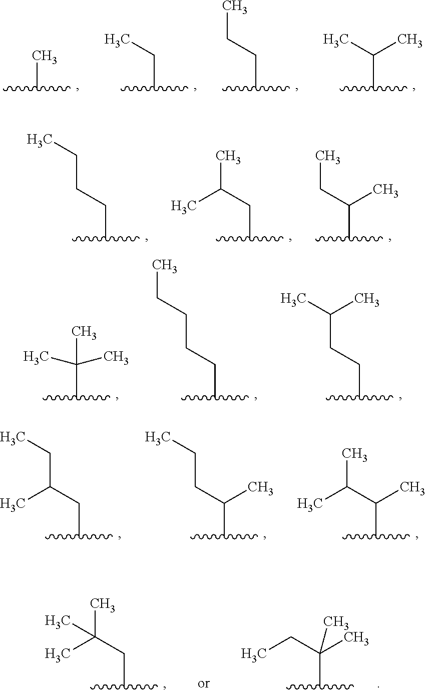

In some embodiments, R³¹ is

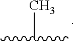

In some embodiments, each of R^{32a} and R^{32b} is, independently,

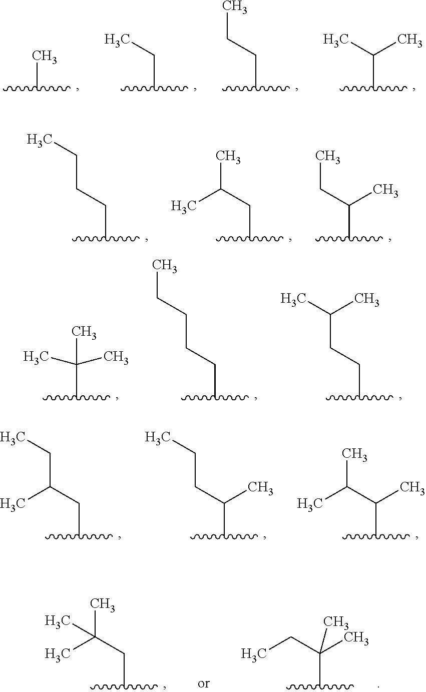

In an aspect, the structural lipid of the invention features a compound having the structure of Formula SX:

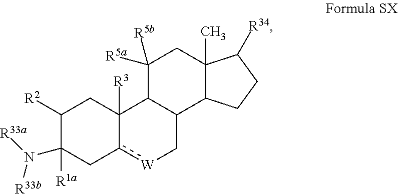

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

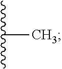

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

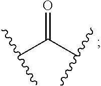 [SMILES: *C(*)=O]

R^{33a} is optionally substituted C₁-C₆ alkyl or

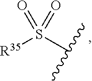

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl, or a pharmaceutically acceptable salt thereof.

In some embodiments, the compound has the structure of Formula SXa:

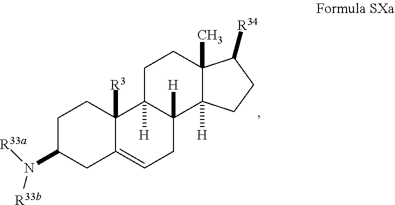

or a pharmaceutically acceptable salt thereof.

In some embodiments, the compound has the structure of Formula SXb:

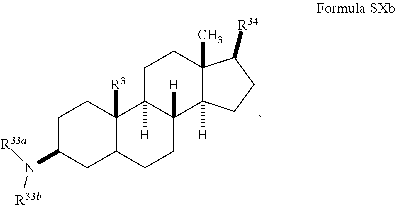

or a pharmaceutically acceptable salt thereof.

In some embodiments, R^{33a} is

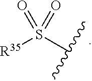

In some embodiments, R³⁵ is

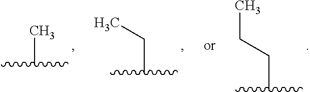

In some embodiments, R³⁵ is

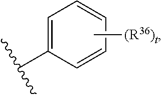

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁴ is

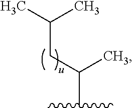

where u is 0, 1, 2, 3, or 4.

In some embodiments, u is 3 or 4.

In an aspect, the structural lipid of the invention features a compound having the structure of Formula SXI:

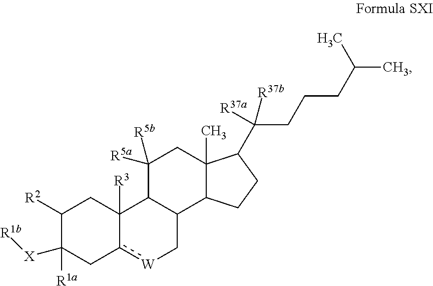

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

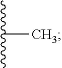

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

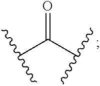 [SMILES: *C(*)=O]

and

each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

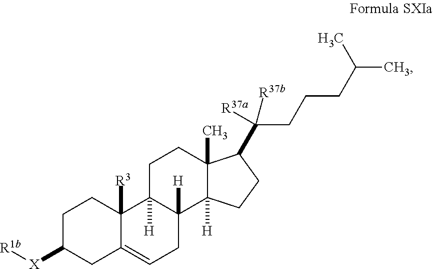

or a pharmaceutically acceptable salt thereof.

In some embodiments, the compound has the structure of Formula SXIb:

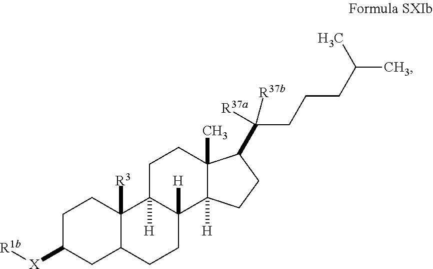

or a pharmaceutically acceptable salt thereof.

In some embodiments, R^{37a} is hydroxyl.

In some embodiments, R^{37b} is

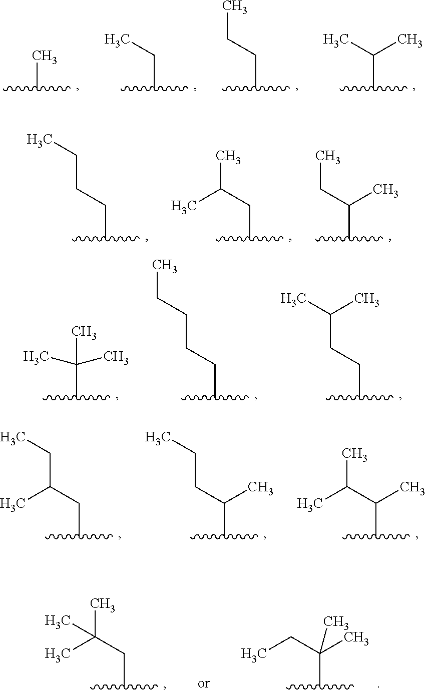

In an aspect, the structural lipid of the invention features a compound having the structure of Formula SXII:

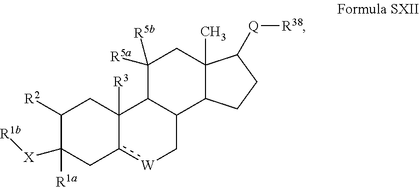

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

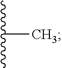

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

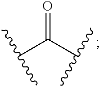 [SMILES: *C(*)=O]

and

Q is O, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula SXIIa:

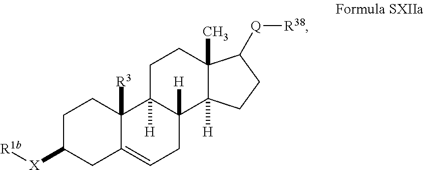

or a pharmaceutically acceptable salt thereof.

In some embodiments, the compound has the structure of Formula SXIIb:

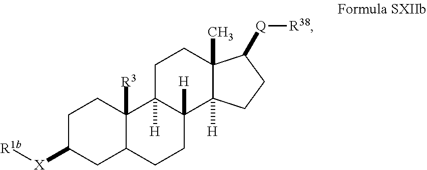

or a pharmaceutically acceptable salt thereof.

In some embodiments, Q is NR^{E}.

In some embodiments, R^{E} is H or

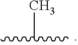

In some embodiments, R^{E} is H. In some embodiments, R^{E} is

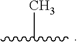

In some embodiments, R³⁸ is

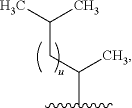

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

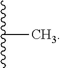

In some embodiments, R^{5a} is H.

In some embodiments, R^{5b} is H.

In an aspect, the invention features a compound having the structure of any one of compounds S-1-42, S-150, S-154, S-162-165, S-169-172 and S-184 in Table 1, or any pharmaceutically acceptable salt thereof. As used herein, “CMPD” refers to “compound.”

TABLE 1
Compounds of Formula SI

| CMPD | |
|---|---|
| No. S- | Structure |
| 1 | 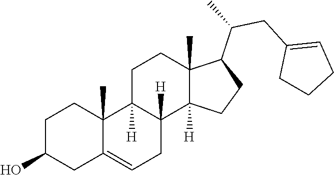 |
| 2 | 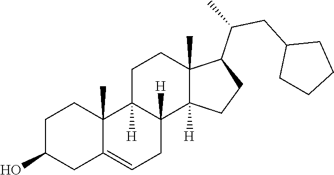 |
| 3 | 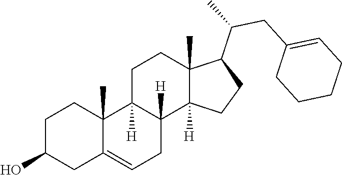 |
| 4 | 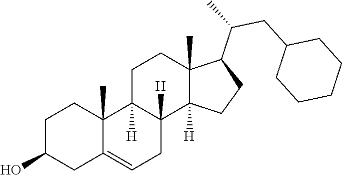 |
| 5 | 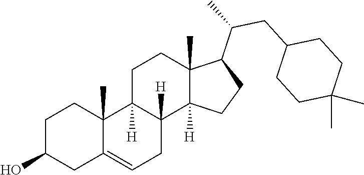 |
| 6 | 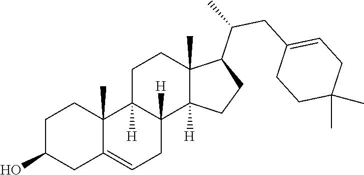 |
| 7 | 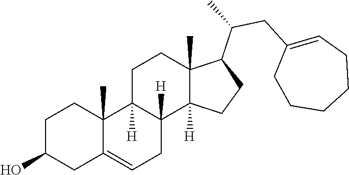 |
| 8 | 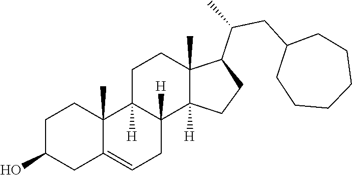 |
| 9 | 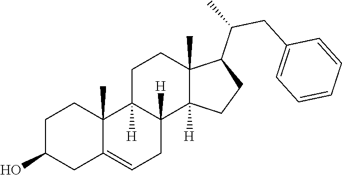 |
| 10 | 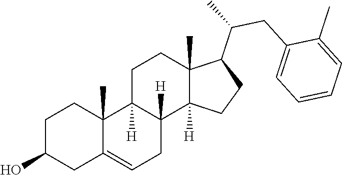 |
| 11 | 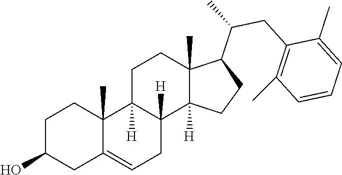 |
| 12 | 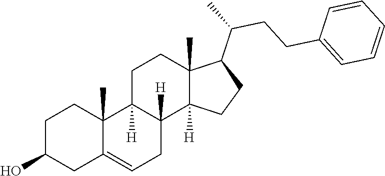 |
| 13 | 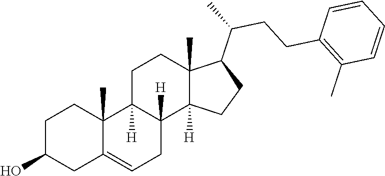 |
| 14 | 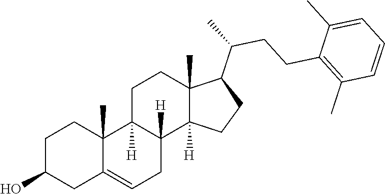 |
| 15 | 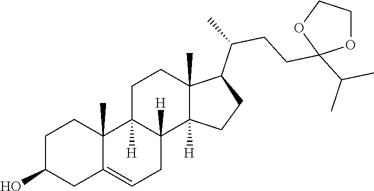 |
| 16 | 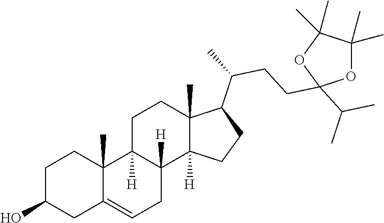 |
| 17 | 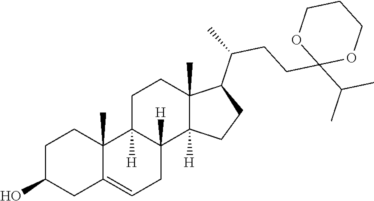 |
| 18 | 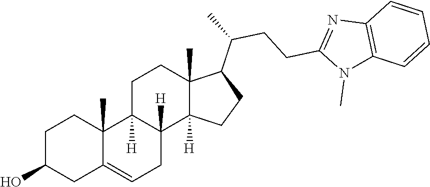 |
| 19 | 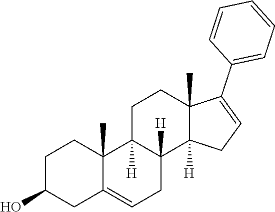 |
| 20 | 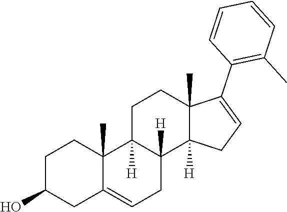 |
| 21 | 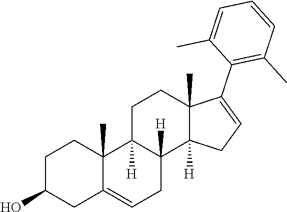 |
| 22 | 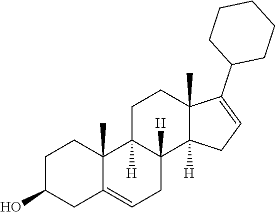 |
| 23 | 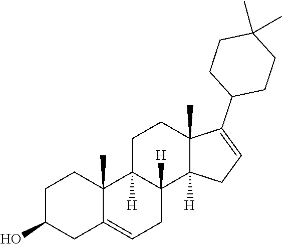 |
| 24 | 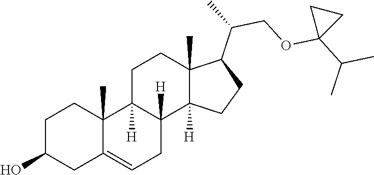 |
| 25 | 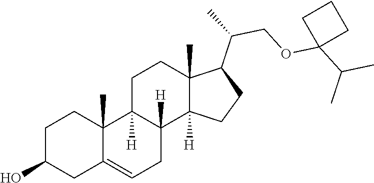 |
| 26 | 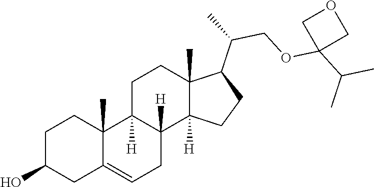 |
| 27 | 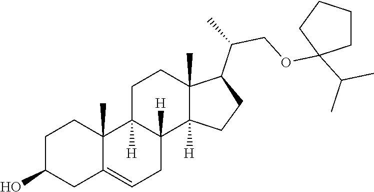 |
| 28 | 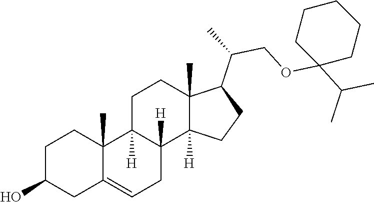 |
| 29 | 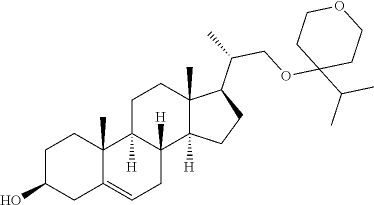 |
| 30 | 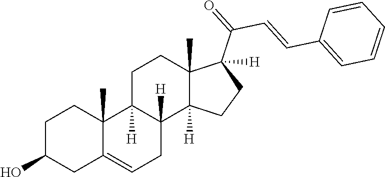 |
| 31 | 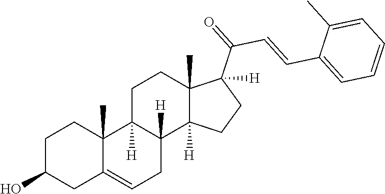 |
| 32 | 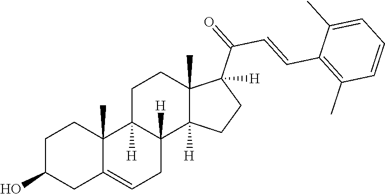 |
| 33 | 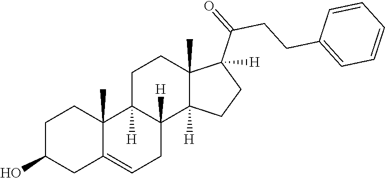 |
| 34 | 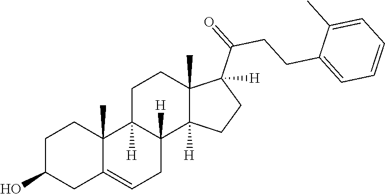 |
| 35 | 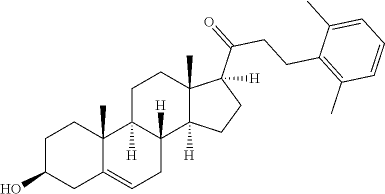 |
| 36 | 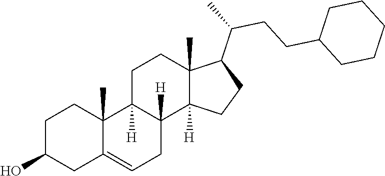 |
| 37 | 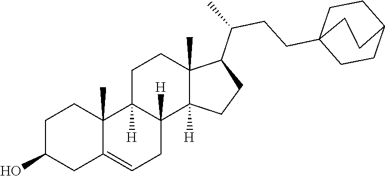 |
| 38 | 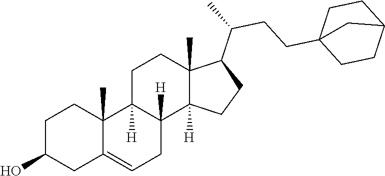 |
| 39 | 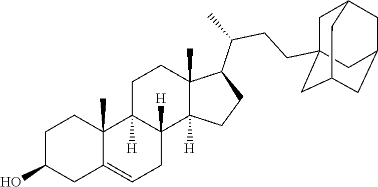 |
| 40 | 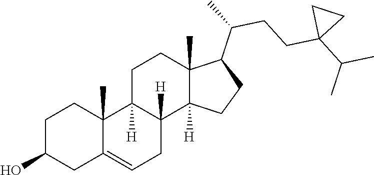 |
| 41 | 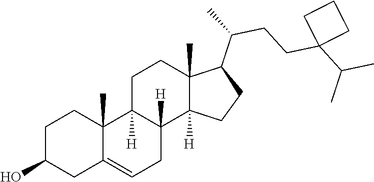 |
| 42 | 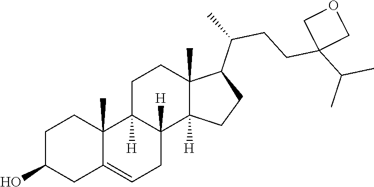 |
| 150 | 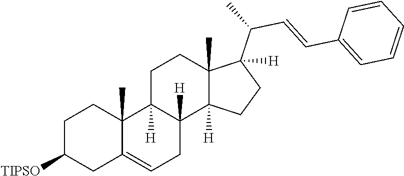 |
| 154 | 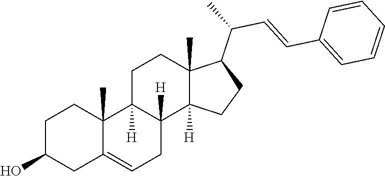 |
| 162 | 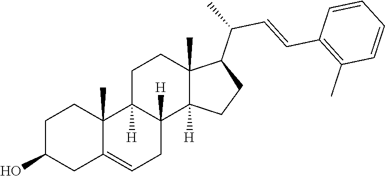 |
| 163 | 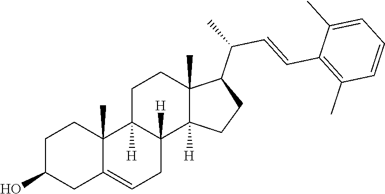 |
| 164 | 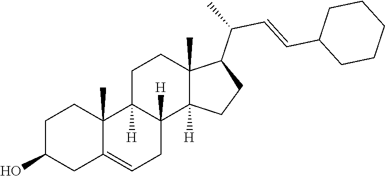 |
| 165 | 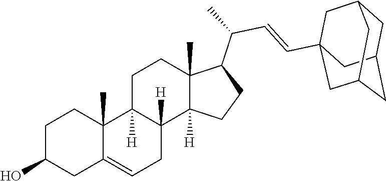 |
| 169 | 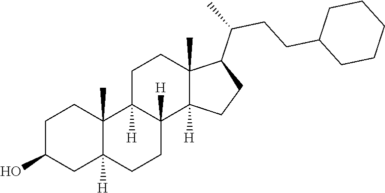 |
| 170 | 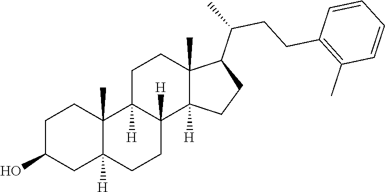 |
| 171 | 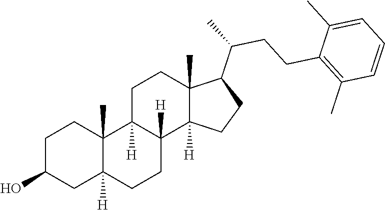 |
| 172 | 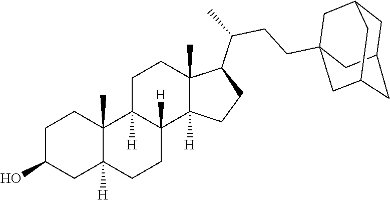 |
| 184 | 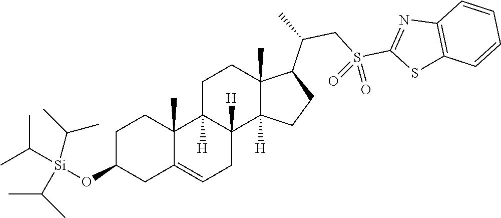 |

In an aspect, the invention features a compound having the structure of any one of compounds S-43-50 and S-175-178 in Table 2, or any pharmaceutically acceptable salt thereof.

TABLE 2
Compounds of Formula SII

| CMPD | |
|---|---|
| No. S- | Structure |
| 43 | 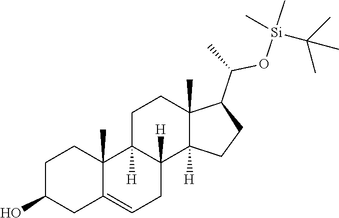 |
| 44 | 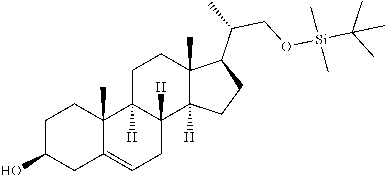 |
| 45 | 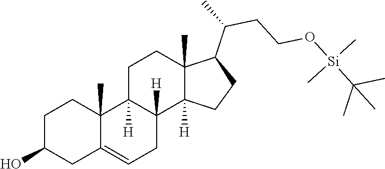 |
| 46 | 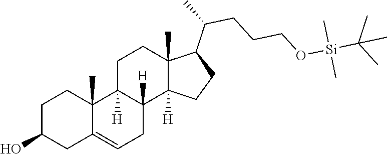 |
| 47 | 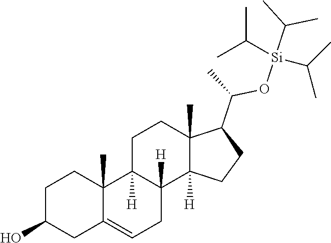 |
| 48 | 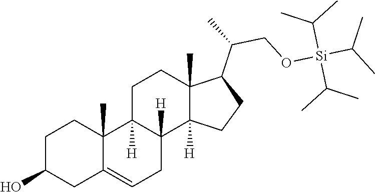 |
| 49 | 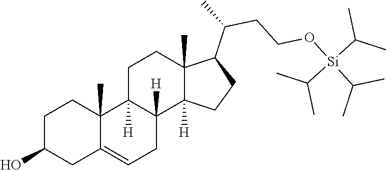 |
| 50 | 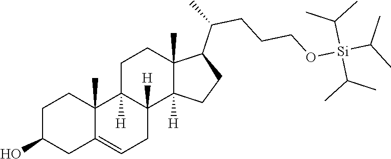 |
| 175 | 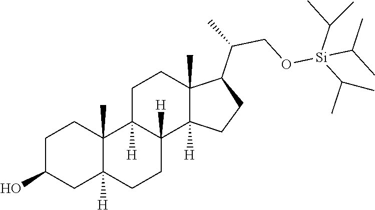 |
| 176 | 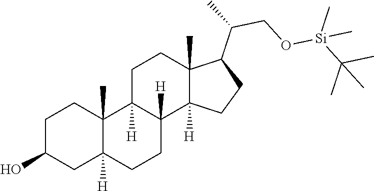 |
| 177 | 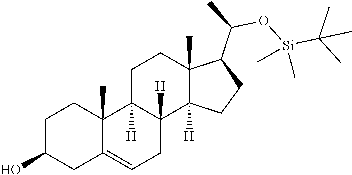 |
| 178 | 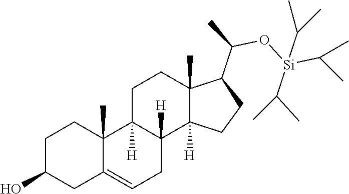 |

In an aspect, the invention features a compound having the structure of any one of compounds S-51-67, S-149 and S-153 in Table 3, or any pharmaceutically acceptable salt thereof.

TABLE 3
Compounds of Formula SIII

| CMPD | |
|---|---|
| No. S- | Structure |
| 51 | 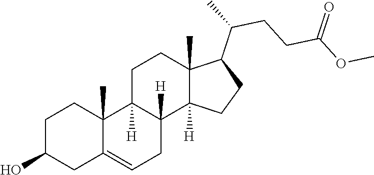 |
| 52 | 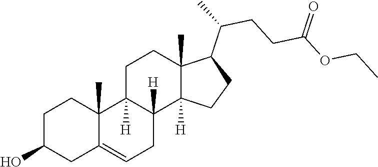 |
| 53 | 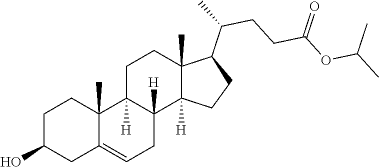 |
| 54 | 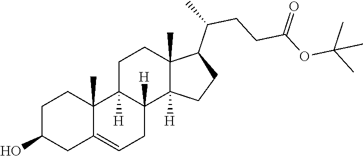 |
| 55 | 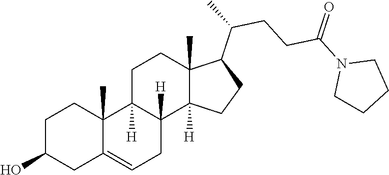 |
| 56 | 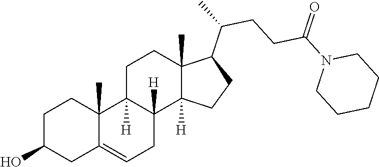 |
| 57 | 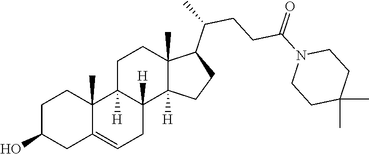 |
| 58 | 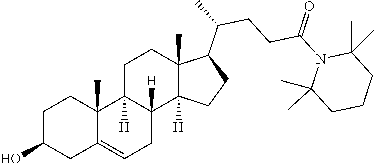 |
| 59 | 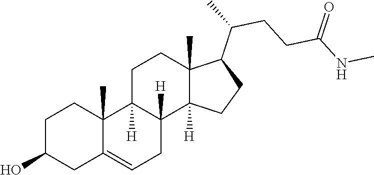 |
| 60 | 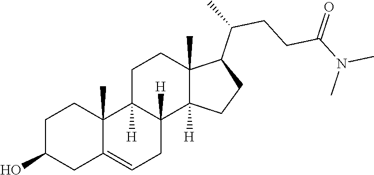 |
| 61 | 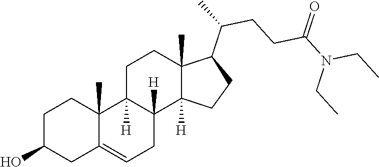 |
| 62 | 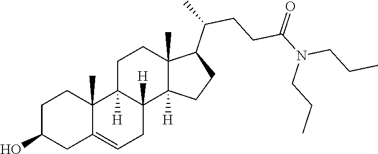 |
| 63 | 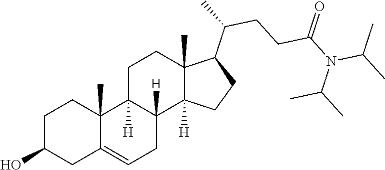 |
| 64 | 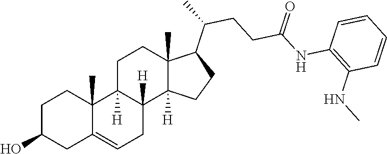 |
| 65 | 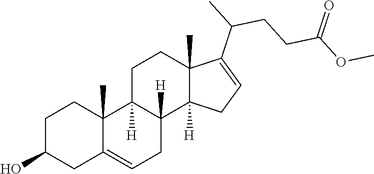 |
| 66 | 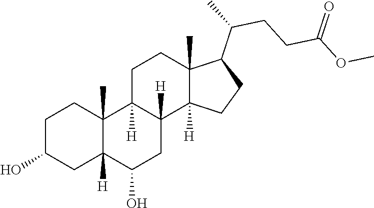 |
| 67 | 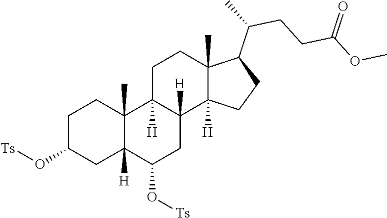 |
| 149 | 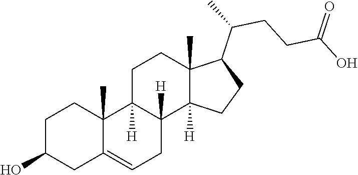 |
| 153 | 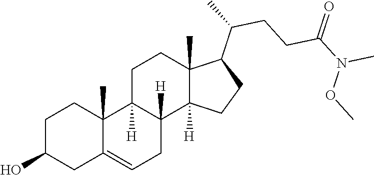 |

In an aspect, the invention features a compound having the structure of any one of compounds S-68-73 in Table 4, or any pharmaceutically acceptable salt thereof.

TABLE 4
Compounds of Formula SIV

| CMPD | |
|---|---|
| No. S- | Structure |
| 68 | 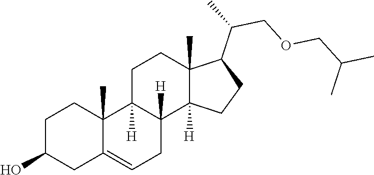 |
| 69 | 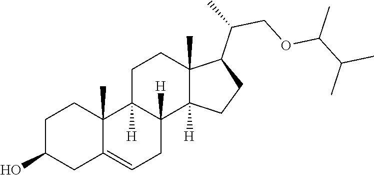 |
| 70 | 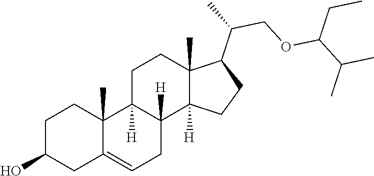 |
| 71 | 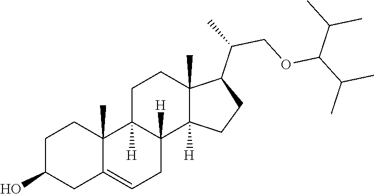 |
| 72 | 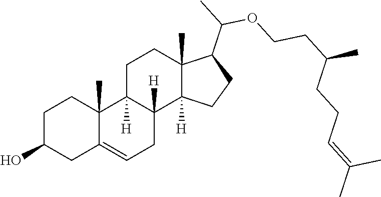 |
| 73 | 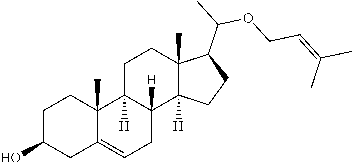 |

In an aspect, the invention features a compound having the structure of any one of compounds S-74-78 in Table 5, or any pharmaceutically acceptable salt thereof.

TABLE 5
Compounds of Formula SV

| CMPD | |
|---|---|
| No. S- | Structure |
| 74 | 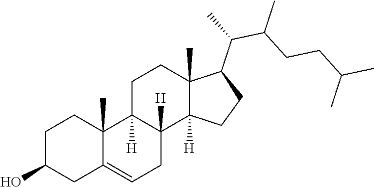 |
| 75 | 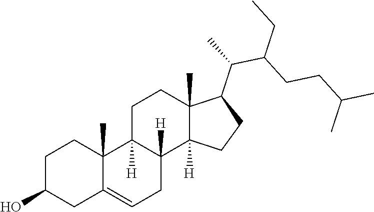 |
| 76 | 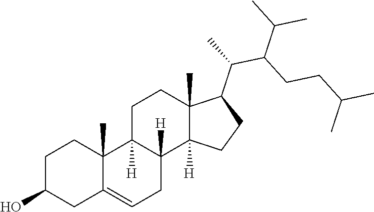 |
| 77 | 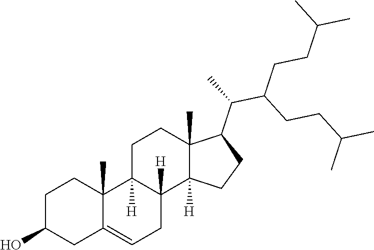 |
| 78 | 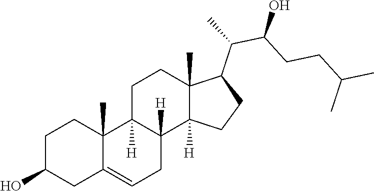 |

In an aspect, the invention features a compound having the structure of any one of compounds S-79 or S-80 in Table 6, or any pharmaceutically acceptable salt thereof.

TABLE 6
Compounds of Formula SVI

| CMPD | |
|---|---|
| No. S- | Structure |
| 79 | 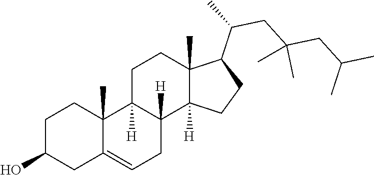 |
| 80 | 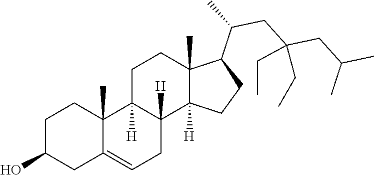 |

In an aspect, the invention features a compound having the structure of any one of compounds S-81-87, S-152 and S-157 in Table 7, or any pharmaceutically acceptable salt thereof.

TABLE 7
Compounds of Formula S-VII

| CMPD | |
|---|---|
| No. S- | Structure |
| 81 | 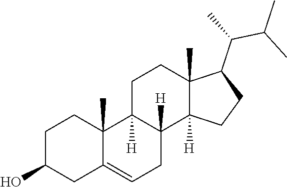 |
| 82 | 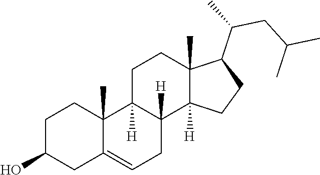 |
| 83 | 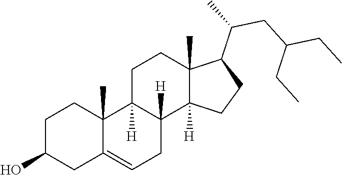 |
| 84 | 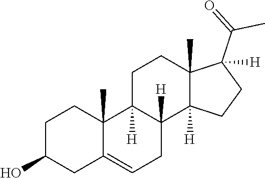 |
| 85 | 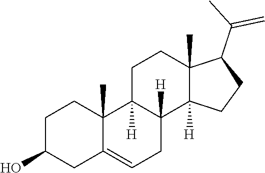 |
| 86 | 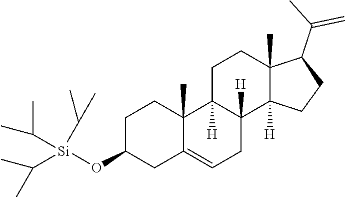 |
| 87 | 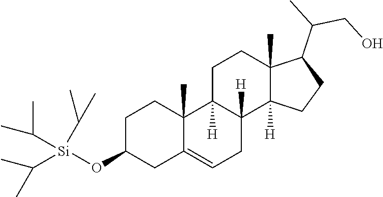 |
| 152 | 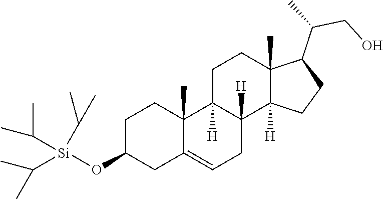 |
| 157 | 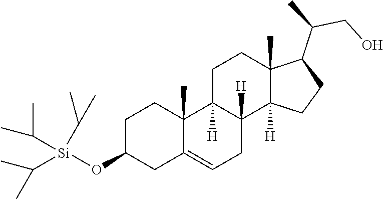 |

In an aspect, the invention features a compound having the structure of any one of compounds S-88-97 in Table 8, or any pharmaceutically acceptable salt thereof.

TABLE 8
Compounds of Formula SVIII

| CMPD | |
|---|---|
| No. S- | Structure |
| 88 | 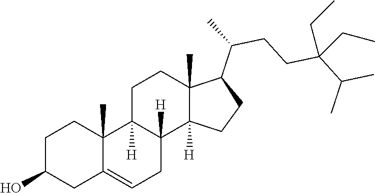 |
| 89 | 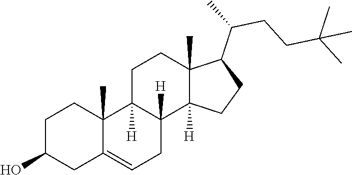 |
| 90 | 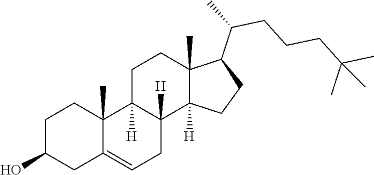 |
| 91 | 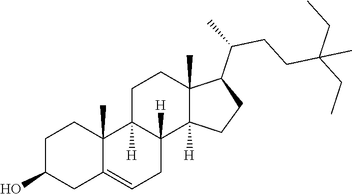 |
| 92 | 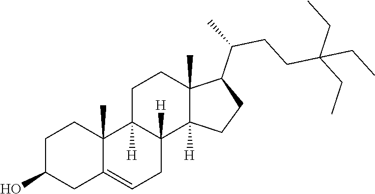 |
| 93 | 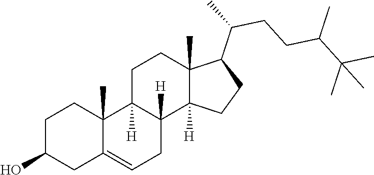 |
| 94 | 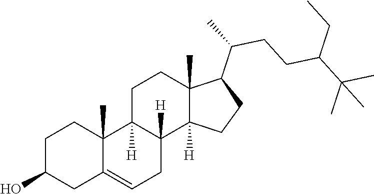 |
| 95 | 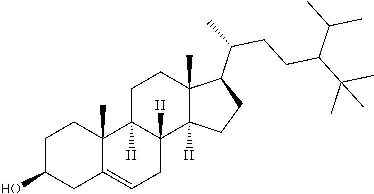 |
| 96 | 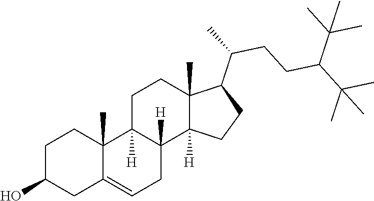 |
| 97 | 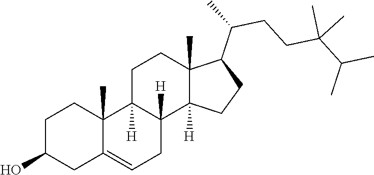 |

In an aspect, the invention features a compound having the structure of any one of compounds S-98-105 and S-180-182 in Table 9, or any pharmaceutically acceptable salt thereof.

TABLE 9
Compounds of Formula SIX

| CMPD | |
|---|---|
| No. S- | Structure |
| 98 | 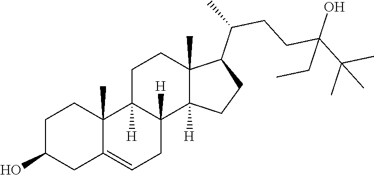 |
| 99 | 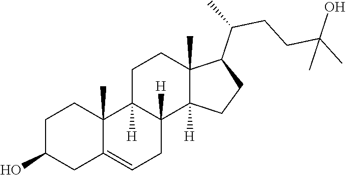 |
| 100 | 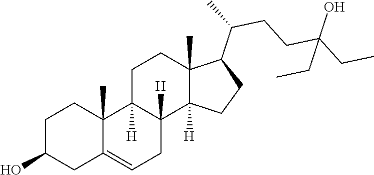 |
| 101 | 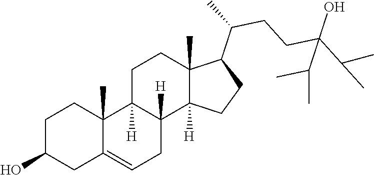 |
| 102 | 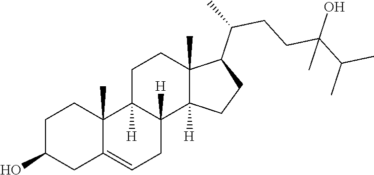 |
| 103 | 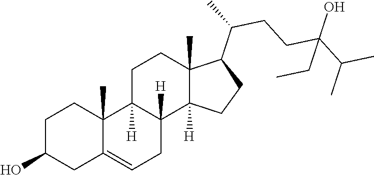 |
| 104 | 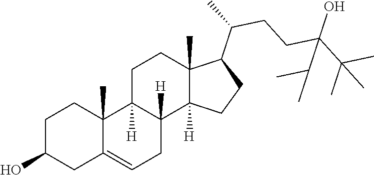 |
| 105 | 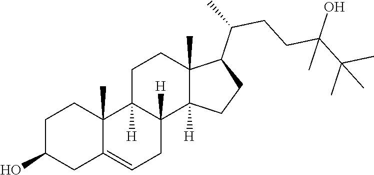 |
| 180 | 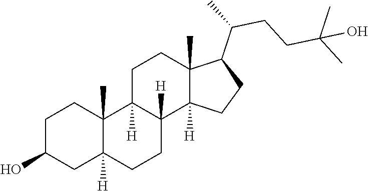 |
| 181 | 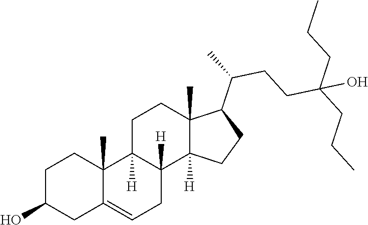 |
| 182 | 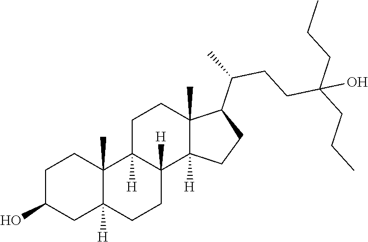 |

In an aspect, the invention features a compound having the structure of compound S-106 in Table 10, or any pharmaceutically acceptable salt thereof.

TABLE 10
Compounds of Formula SX

| CMPD | |
|---|---|
| No. S- | Structure |
| 106 | 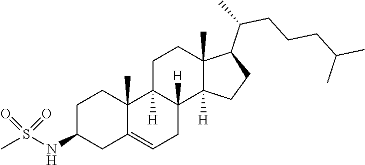 |

In an aspect, the invention features a compound having the structure of compound S-107 or S-108 in Table 11, or any pharmaceutically acceptable salt thereof.

TABLE 11
Compounds of Formula SXI

| CMPD | |
|---|---|
| No. S- | Structure |
| 107 | 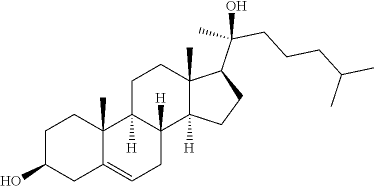 |
| 108 | 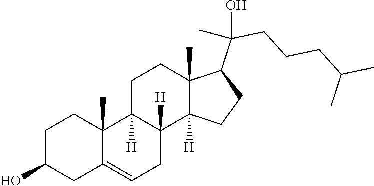 |

In an aspect, the invention features a compound having the structure of compound S-109 in Table 12, or any pharmaceutically acceptable salt thereof.

TABLE 12
Compounds of Formula SXII

| CMPD | |
|---|---|
| No. S- | Structure |
| 109 | 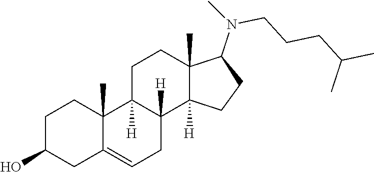 |

In an aspect, the invention features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Invention

| CMPD | |
|---|---|
| No. S- | Structure |
| 110 | 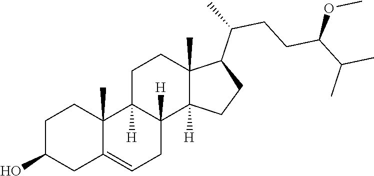 |
| 111 | 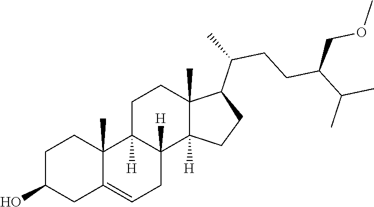 |
| 112 | 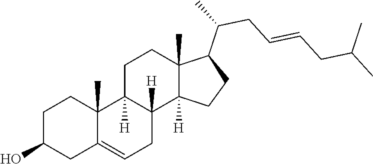 |
| 113 | 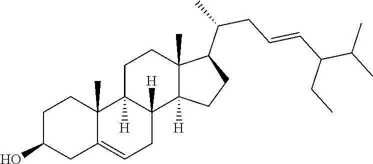 |
| 114 | 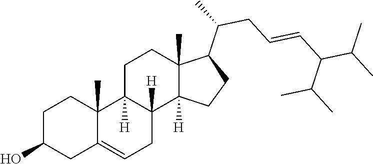 |
| 115 | 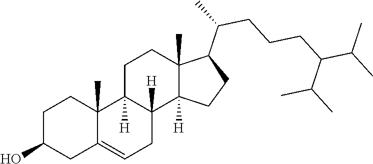 |
| 116 | 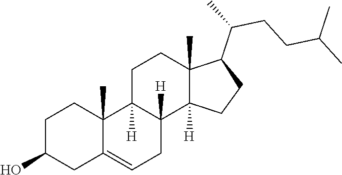 |
| 117 | 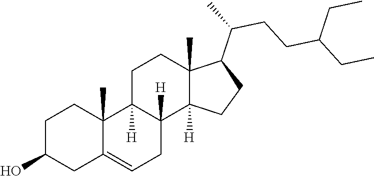 |
| 118 | 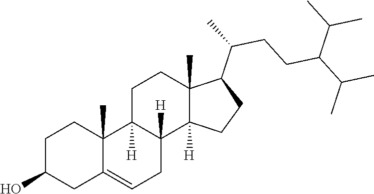 |
| 119 | 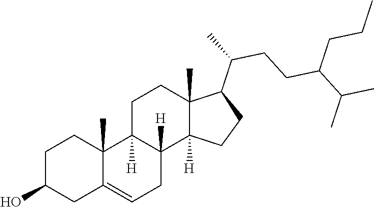 |
| 120 | 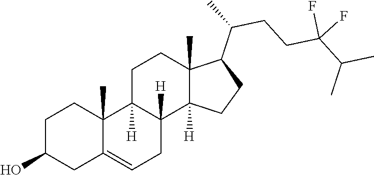 |
| 121 | 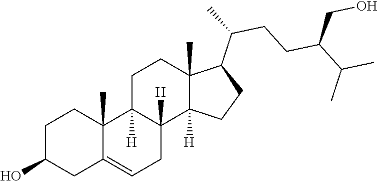 |
| 122 | 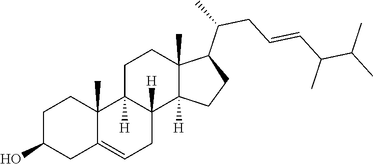 |
| 123 | 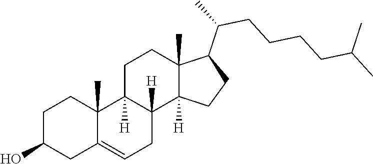 |
| 124 | 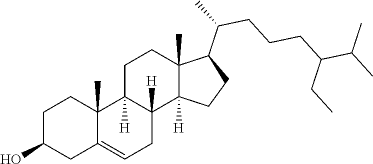 |
| 125 | 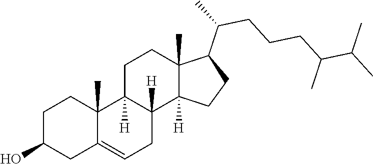 |
| 126 | 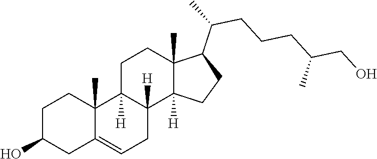 |
| 127 | 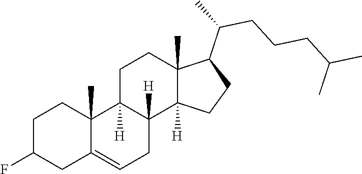 |
| 128 | 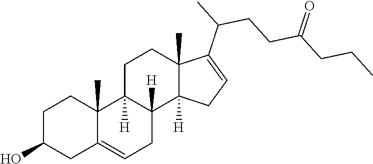 |
| 129 | 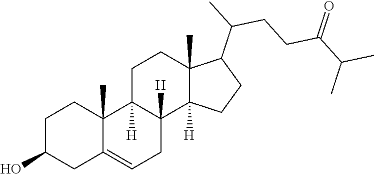 |
| 130 | 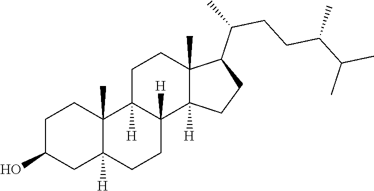 |
| 155 | 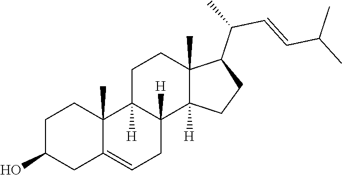 |
| 156 | 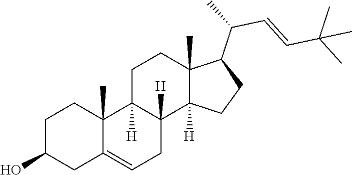 |
| 158 | 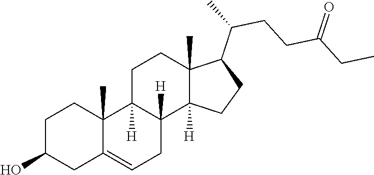 |
| 160 | 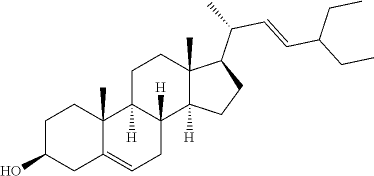 |
| 161 | 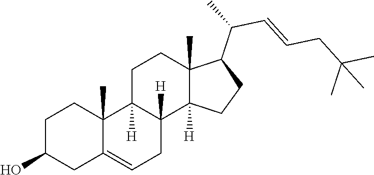 |
| 166 | 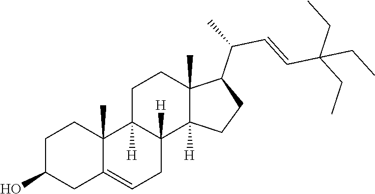 |
| 167 | 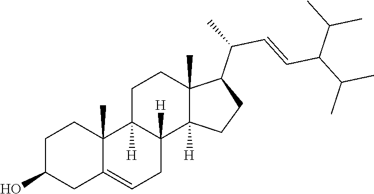 |
| 168 | 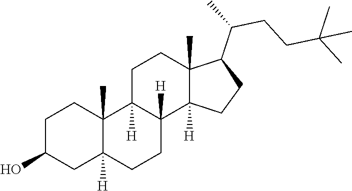 |
| 173 | 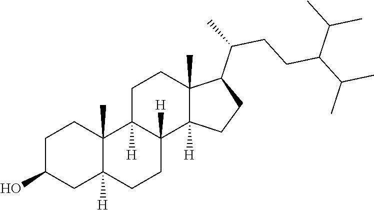 |
| 174 | 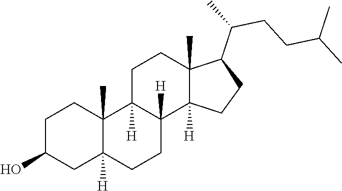 |
| 179 | 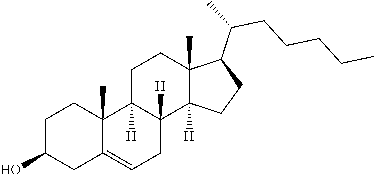 |

In an aspect, the invention features a compound having the structure of any one of compounds S-131-133 in Table 14, or any pharmaceutically acceptable salt thereof.

TABLE 14
Compounds of the Invention

| CMPD | |
|---|---|
| No. S- | Structure |
| 131 | 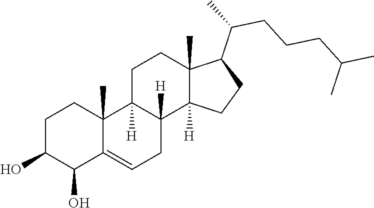 |
| 132 | 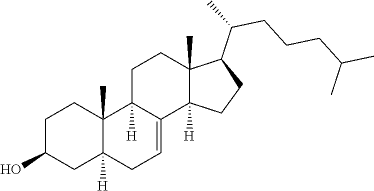 |
| 133 | 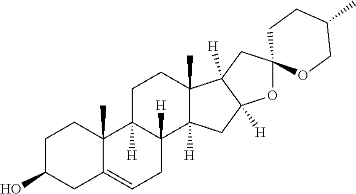 |

In an aspect, the invention features a compound having the structure of any one of compounds S-134-148, S-151 and S-159 in Table 15, or any pharmaceutically acceptable salt thereof.

TABLE 15
Compounds of the Invention

| CMPD | |
|---|---|
| No. S- | Structure |
| 134 | 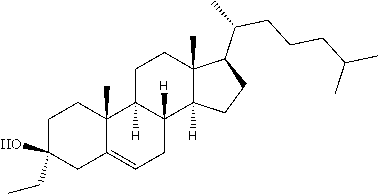 |
| 135 | 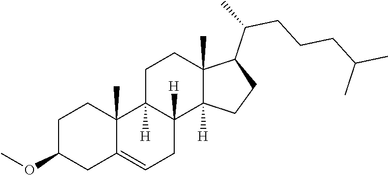 |
| 136 | 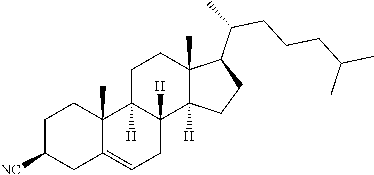 |
| 137 | 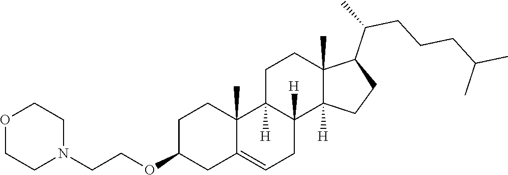 |
| 138 | 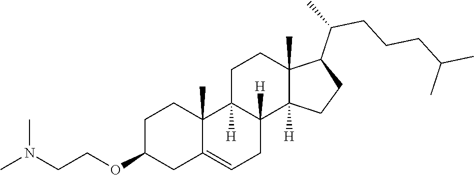 |
| 139 | 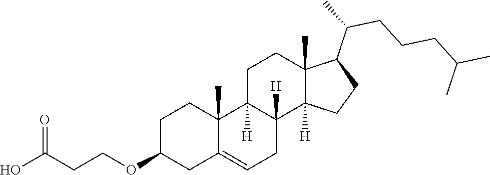 |
| 140 | 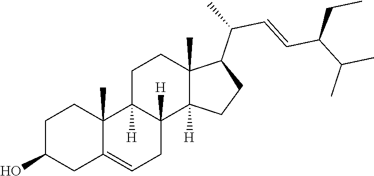 |
| 141 | 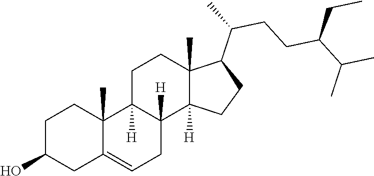 |
| 142 | 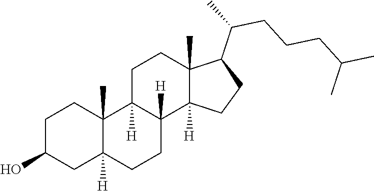 |
| 143 | 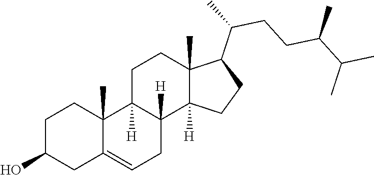 |
| 144 | 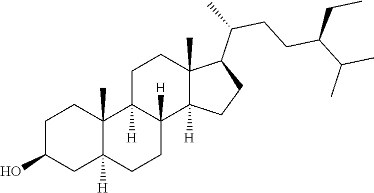 |
| 145 | 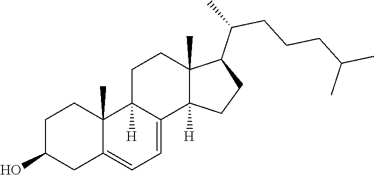 |
| 146 | 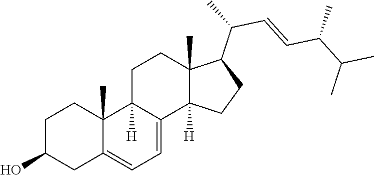 |
| 147 | 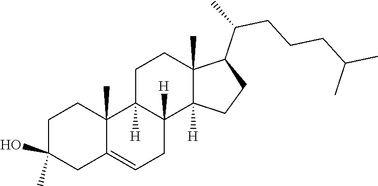 |
| 148 | 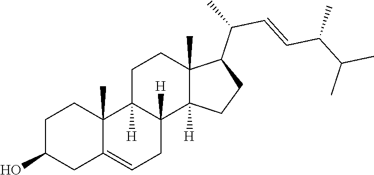 |
| 151 | 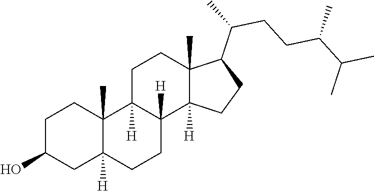 |
| 159 | 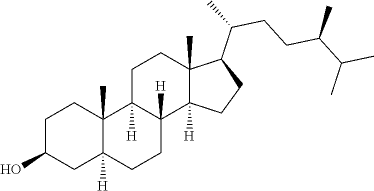 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one or more structural lipids of the lipid nanoparticles of the invention can be composition 183 in Table 16.

TABLE 16
Structural Lipid Compositions

| Compo- | |
|---|---|
| sition | |
| S- No. | Structure |
| 183 | 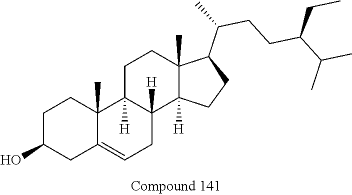 |
| | 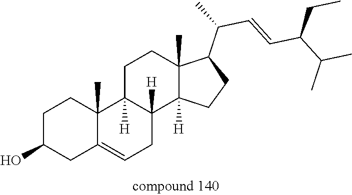 |
| | 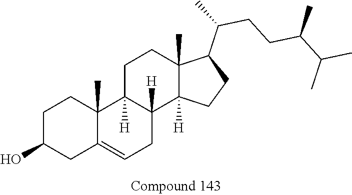 |
| | 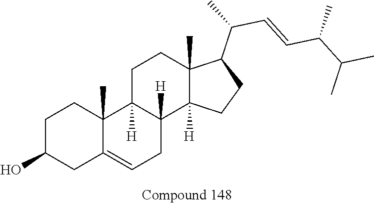 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, A5-avenaserol, A7-avenaserol or a A7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds S-1-148 combined with a cholesterol and/or a phytosterol.

For example, the structural component of the lipid nanoparticle can be a mixture of one or more structural compounds (e.g. any of Compounds S-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(iii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

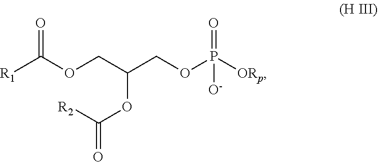

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanol amine (PE 18:3 (9Z,12Z, 15Z), 1,2-diarachidonoyl-sn-glycero-3-phosphoethanolamine (DAPE 18:3 (9Z,12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin. Each possibility represents a separate embodiment of the invention.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in an immune cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

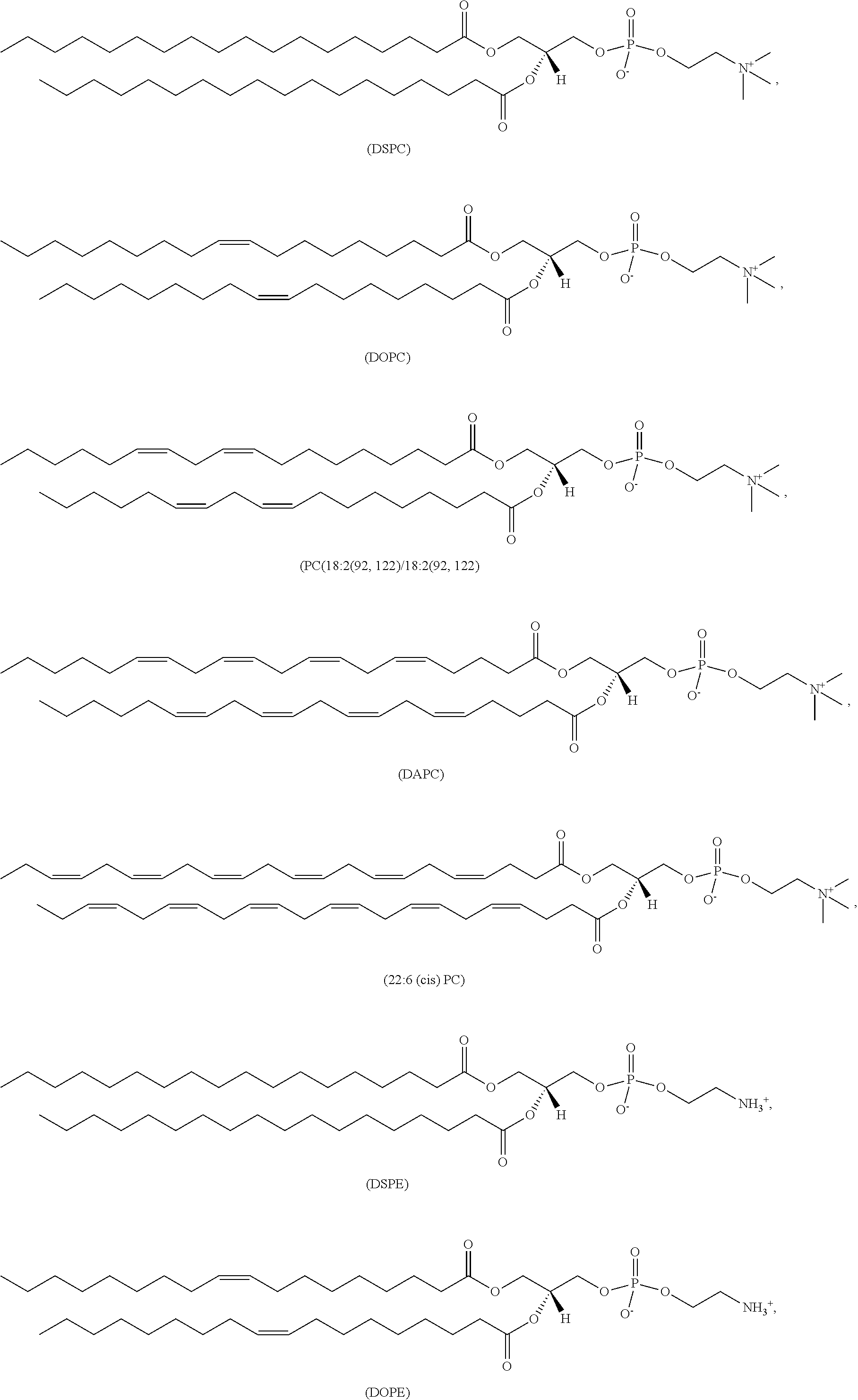

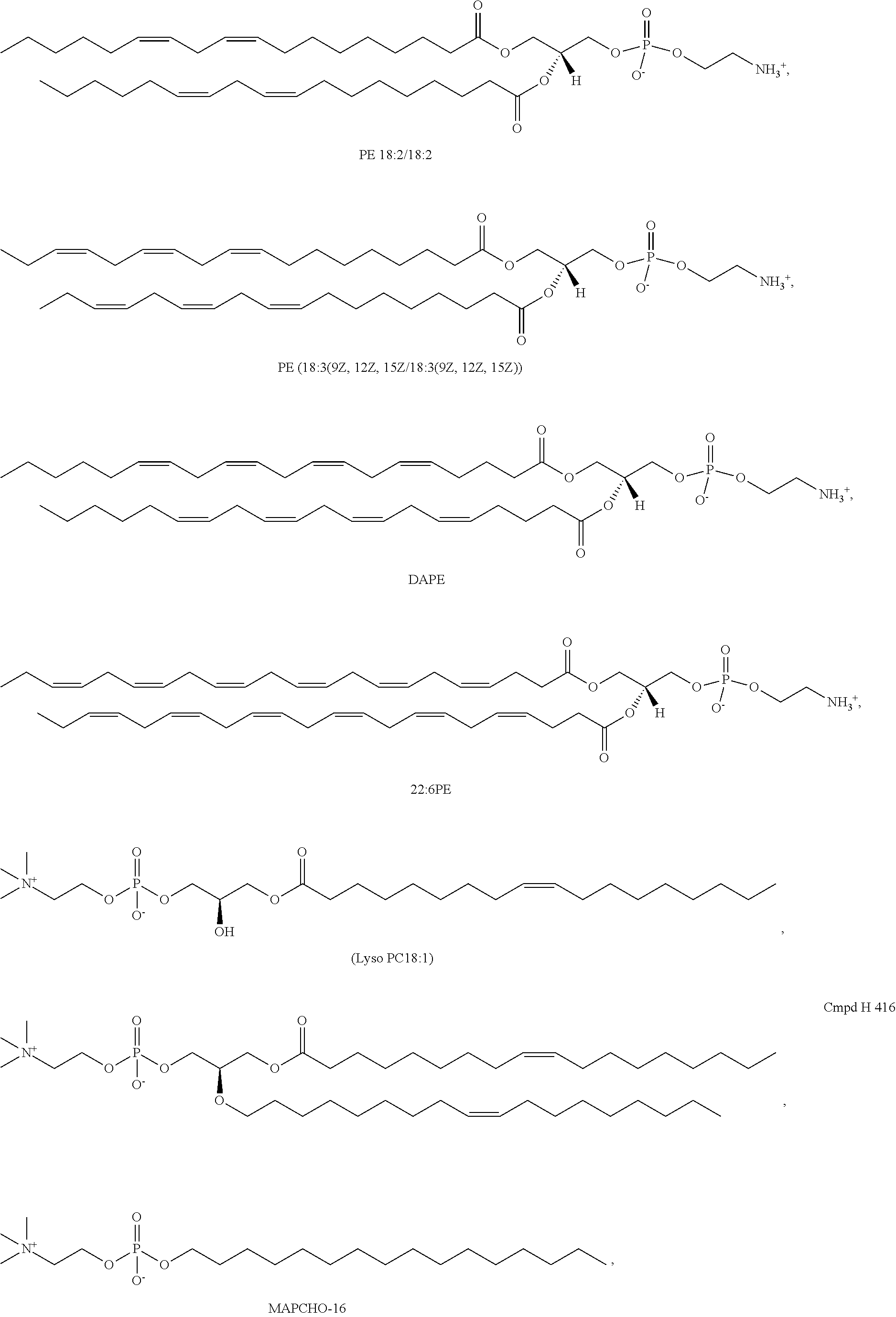

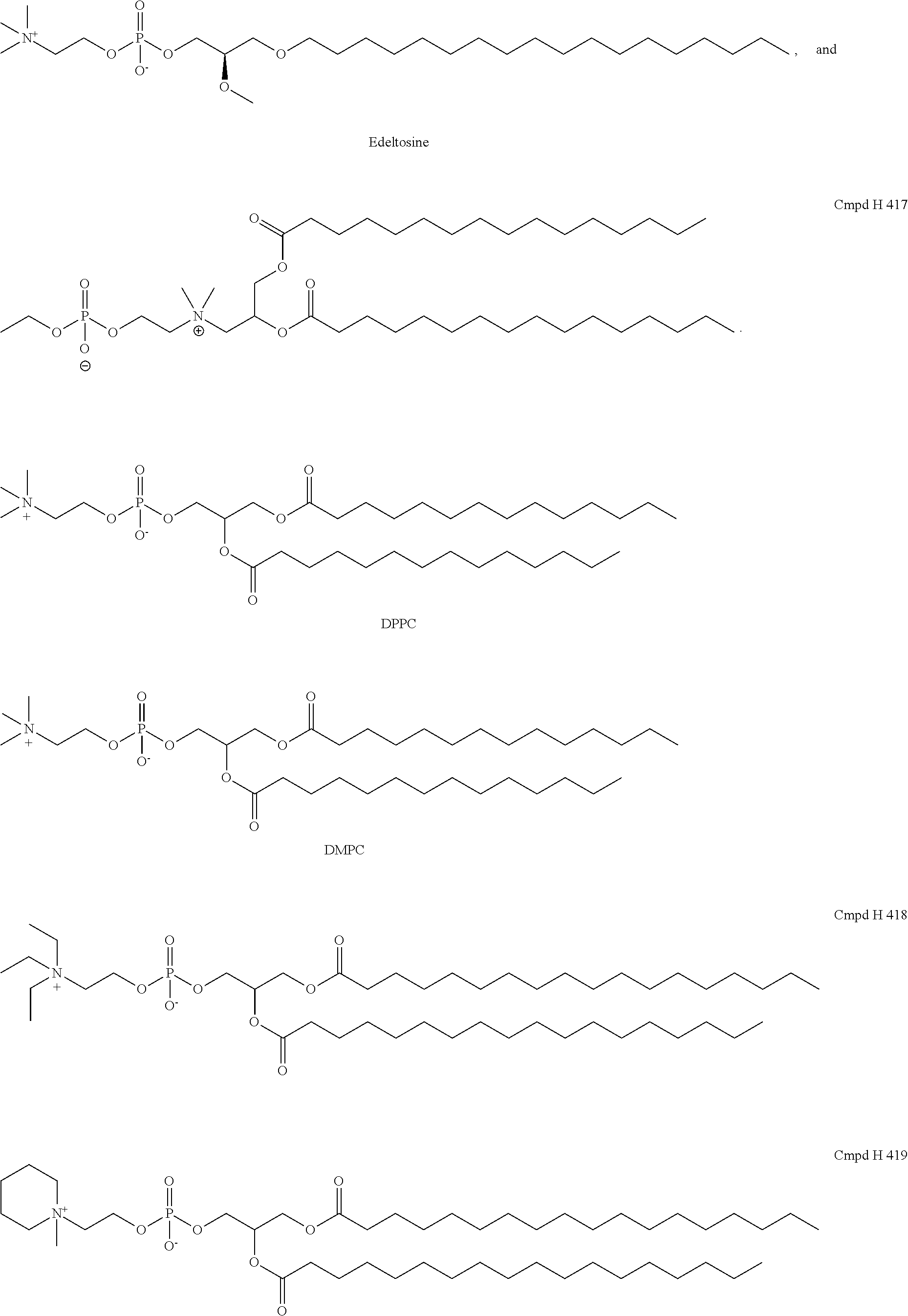

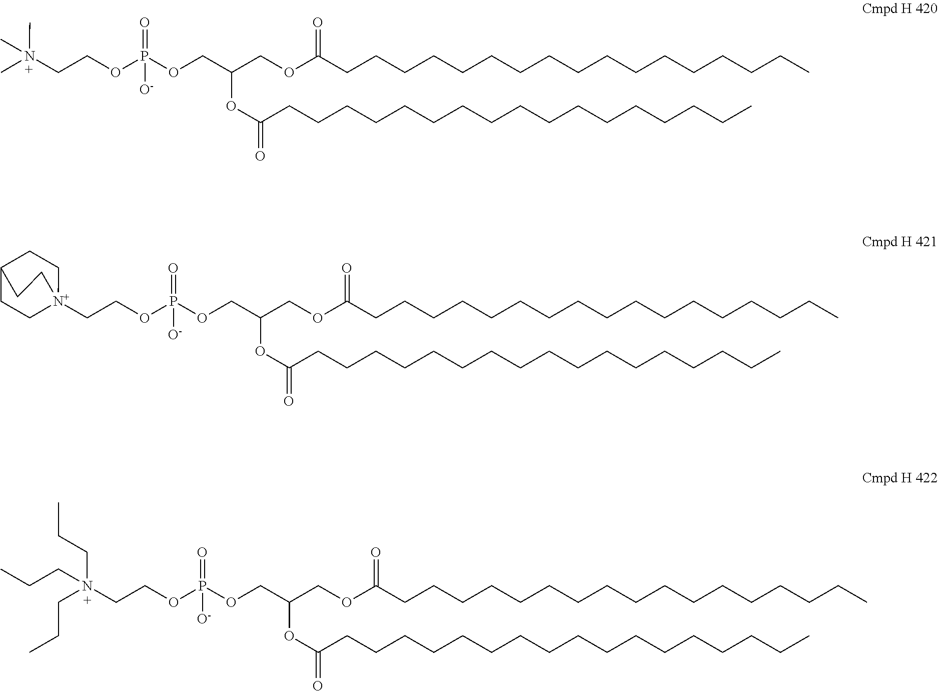

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

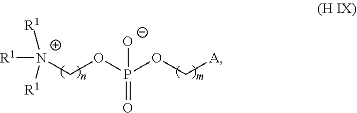

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

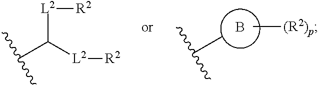

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

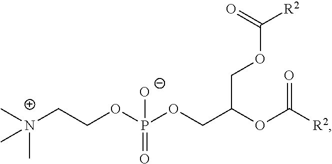

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

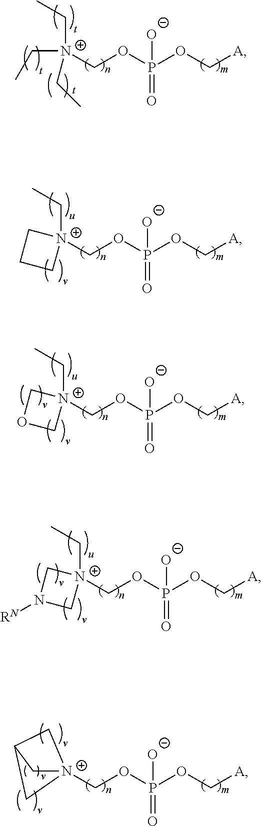

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (H IX) is of one of the following formulae:

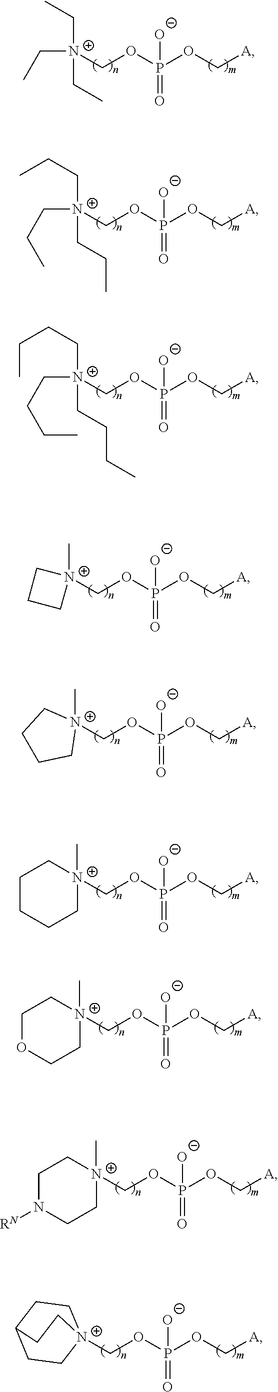

or a salt thereof.

In certain embodiments, a compound of Formula (H IX) is one of the following:

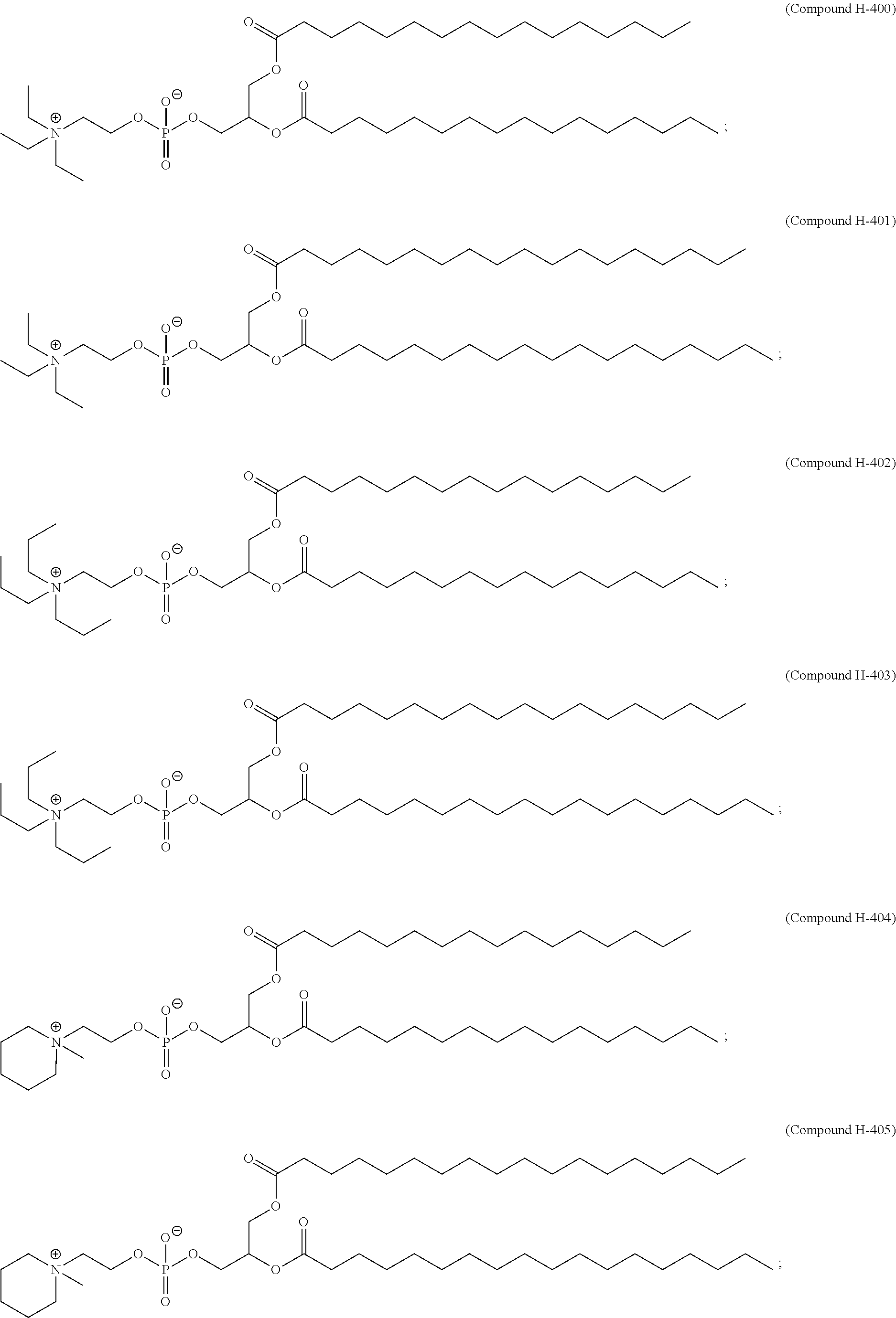

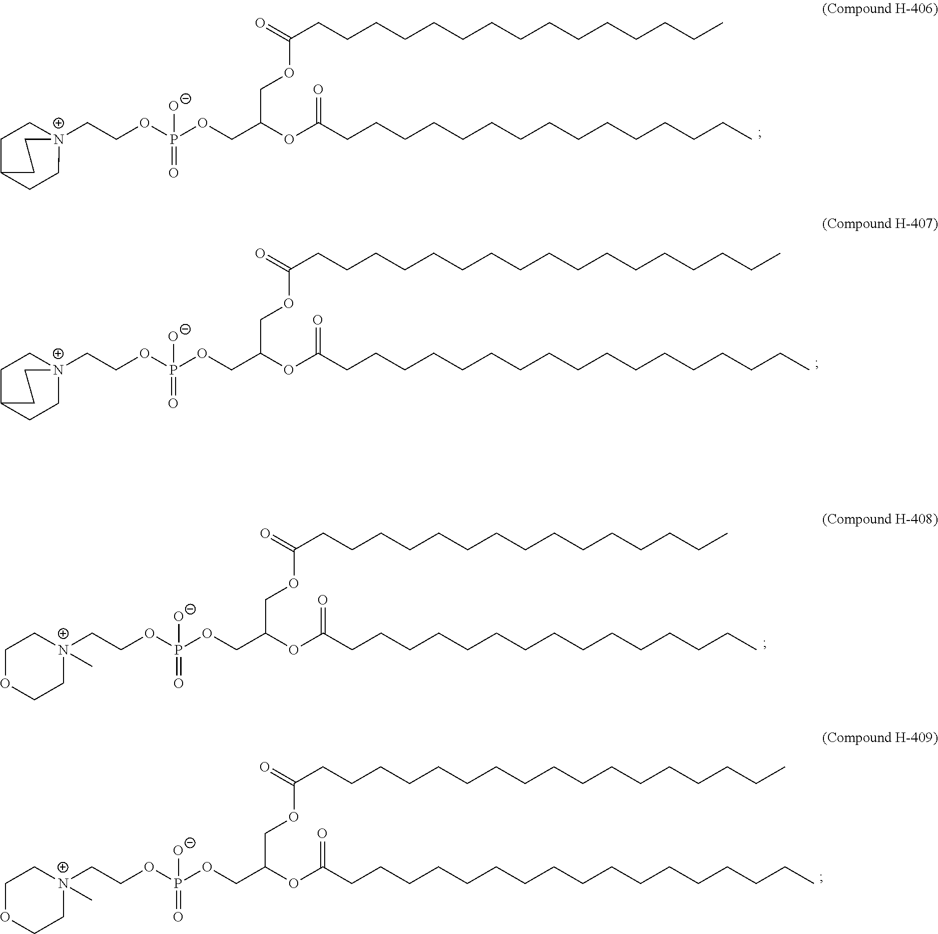 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In one embodiment, an immune cell delivery LNP comprises Compound H-409 as a non-cationic helper lipid.

(ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c):

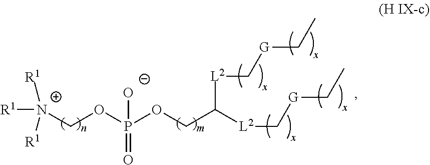

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

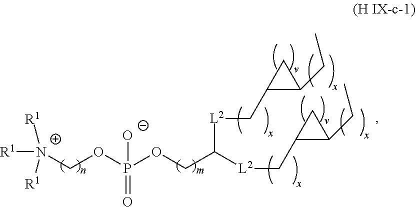

or salt thereof, wherein:
each instance of v is independently 1, 2, or 3.

In certain embodiments, the compound of Formula (H IX-c) is of Formula (H IX-c-2):

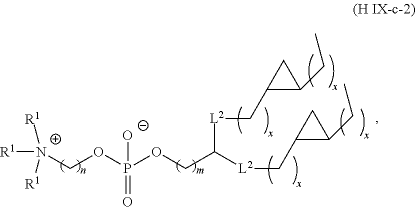

or a salt thereof.

In certain embodiments, the compound of Formula (IX-c) is of the following formula:

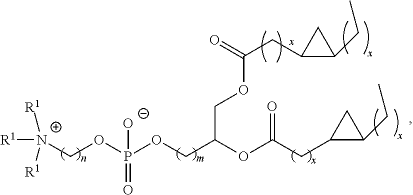

or a salt thereof.

In certain embodiments, the compound of Formula (H IX-c) is the following:

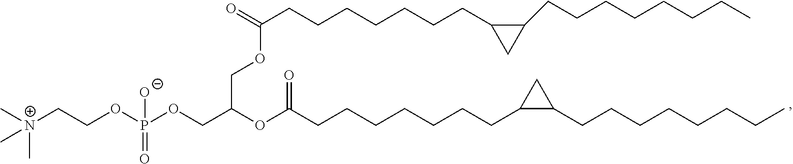 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

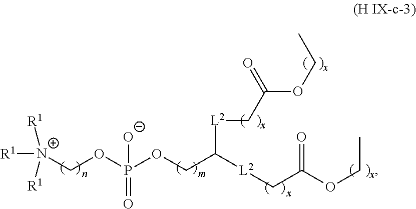

or a salt thereof.

In certain embodiments, the compound of Formula (H IX-c) is of the following formulae:

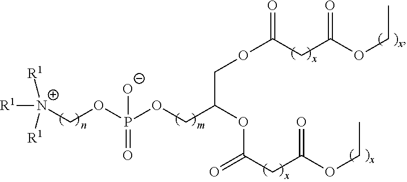

or a salt thereof.

In certain embodiments, the compound of Formula (H IX-c) is the following:

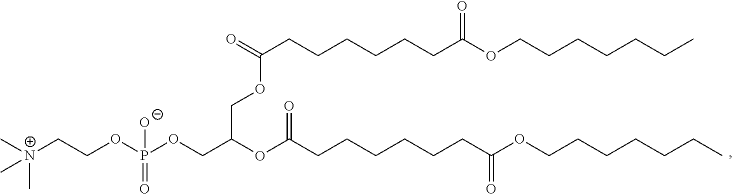 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

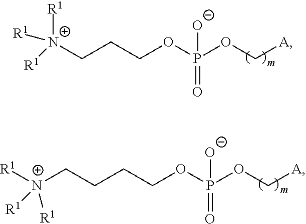

or a salt thereof.

In certain embodiments, a compound of Formula (H IX) is one of the following:

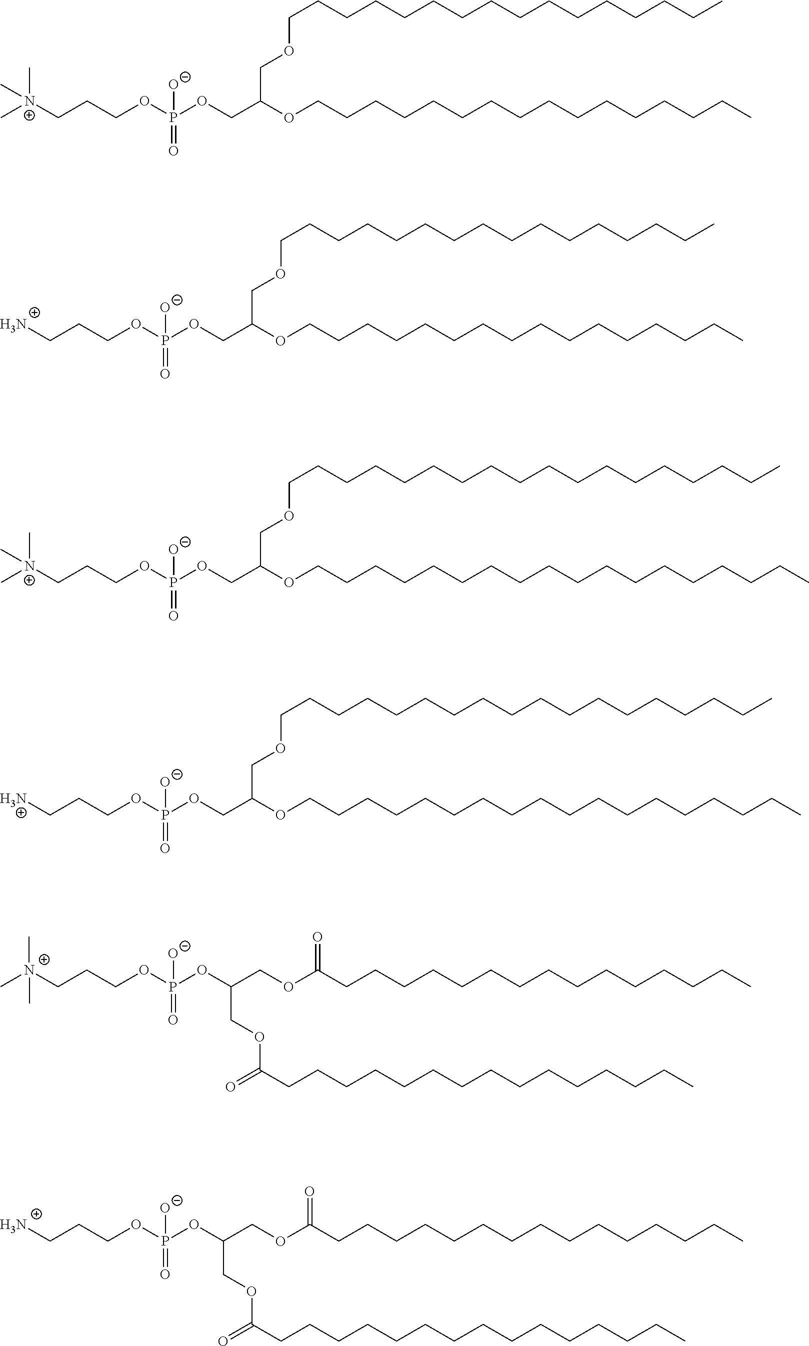

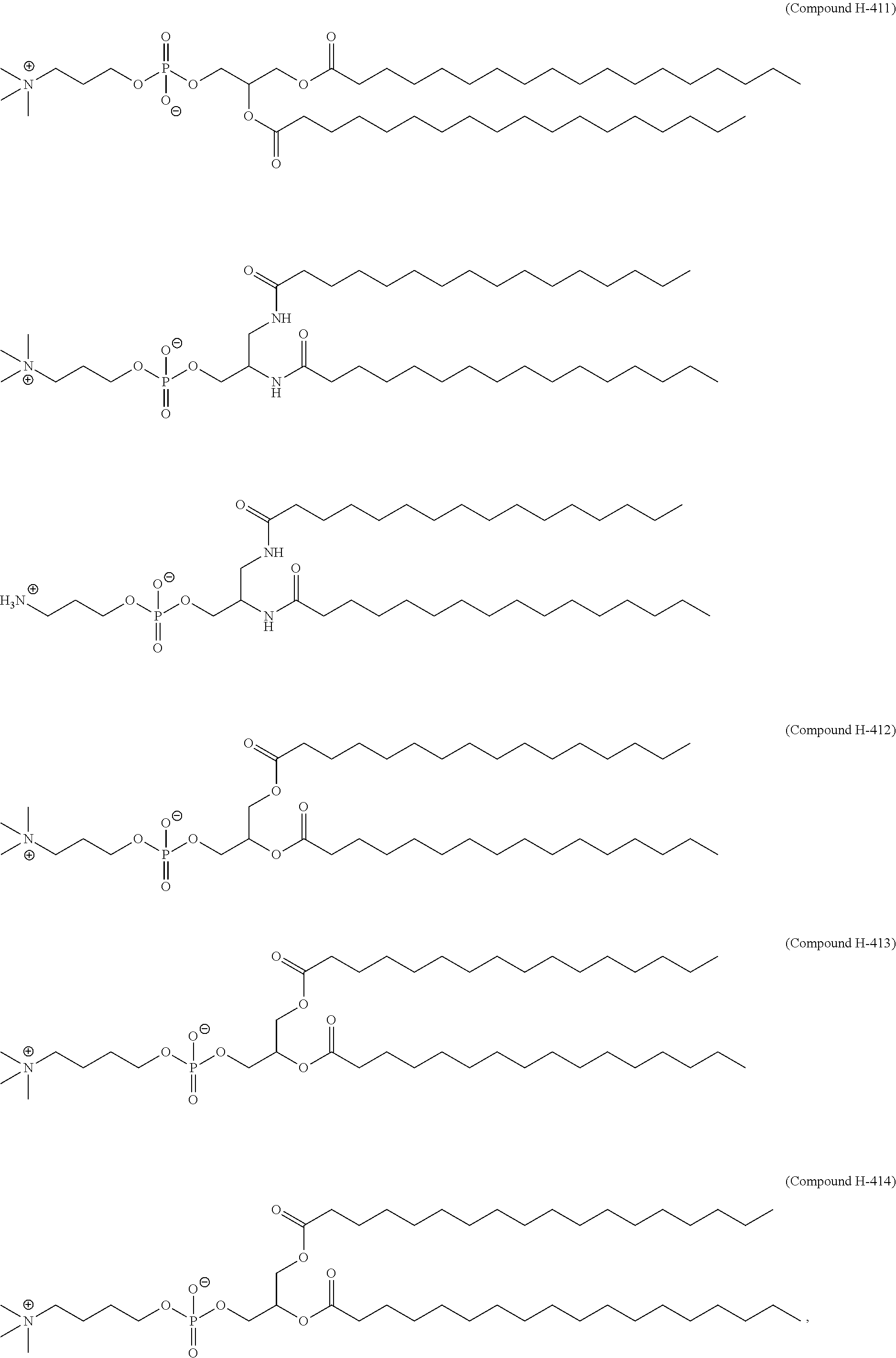

or salts thereof.

In certain embodiments, an alternative lipid is used in place of a phospholipid of the invention. Non-limiting examples of such alternative lipids include the following:

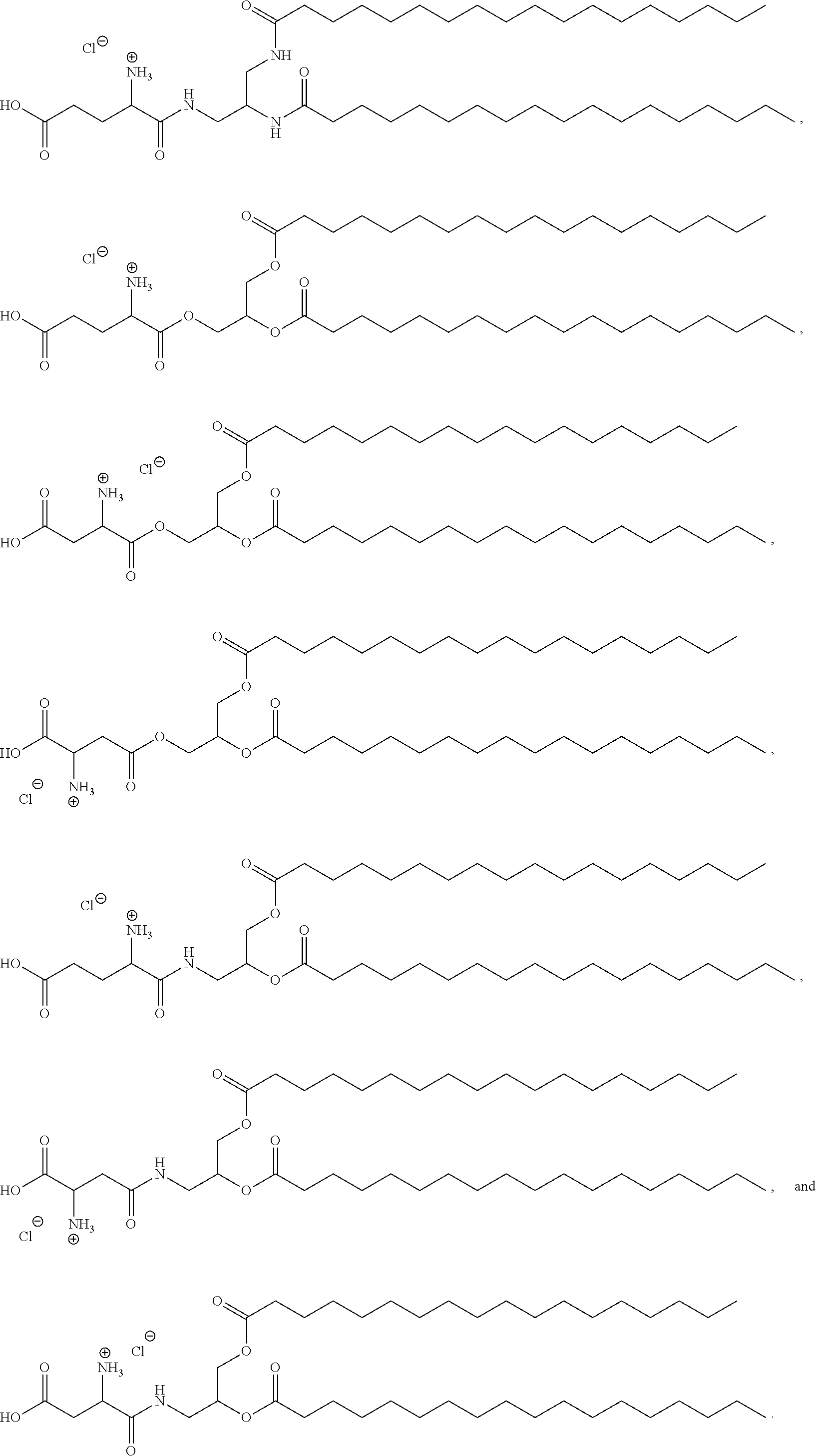

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

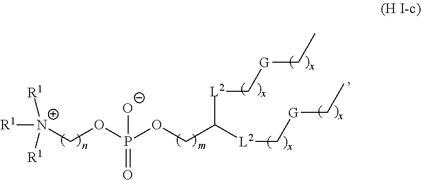

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

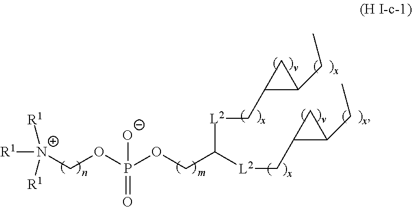

or salt thereof, wherein:

each instance of v is independently 1, 2, or 3.

In certain embodiments, the compound of Formula (H I-c) is of Formula (H I-c-2):

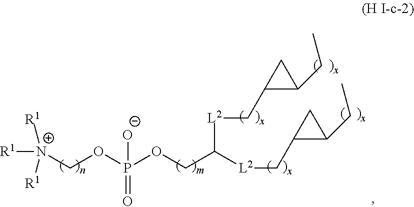

or a salt thereof.

In certain embodiments, the compound of Formula (I-c) is of the following formula:

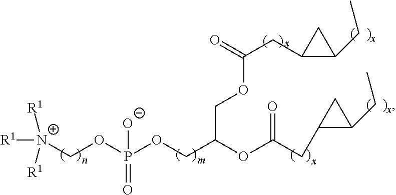

or a salt thereof.

In certain embodiments, the compound of Formula (H I-c) is the following:

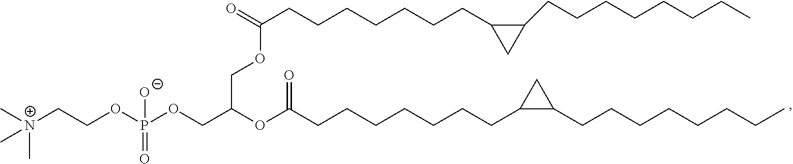 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

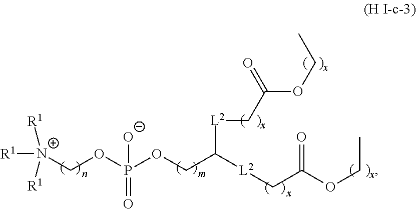

or a salt thereof.

In certain embodiments, the compound of Formula (H I-c) is of the following formulae:

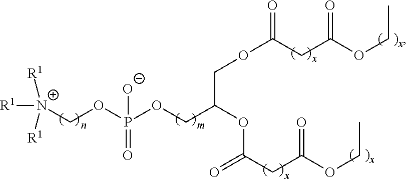

or a salt thereof.

In certain embodiments, the compound of Formula (H I-c) is the following:

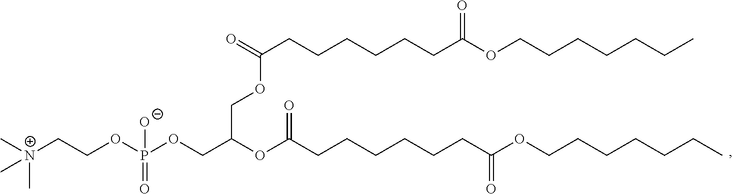 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

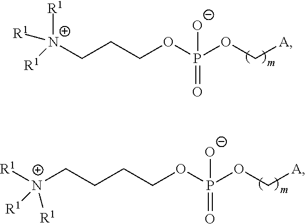

or a salt thereof.

In certain embodiments, a compound of Formula (H I) is one of the following:

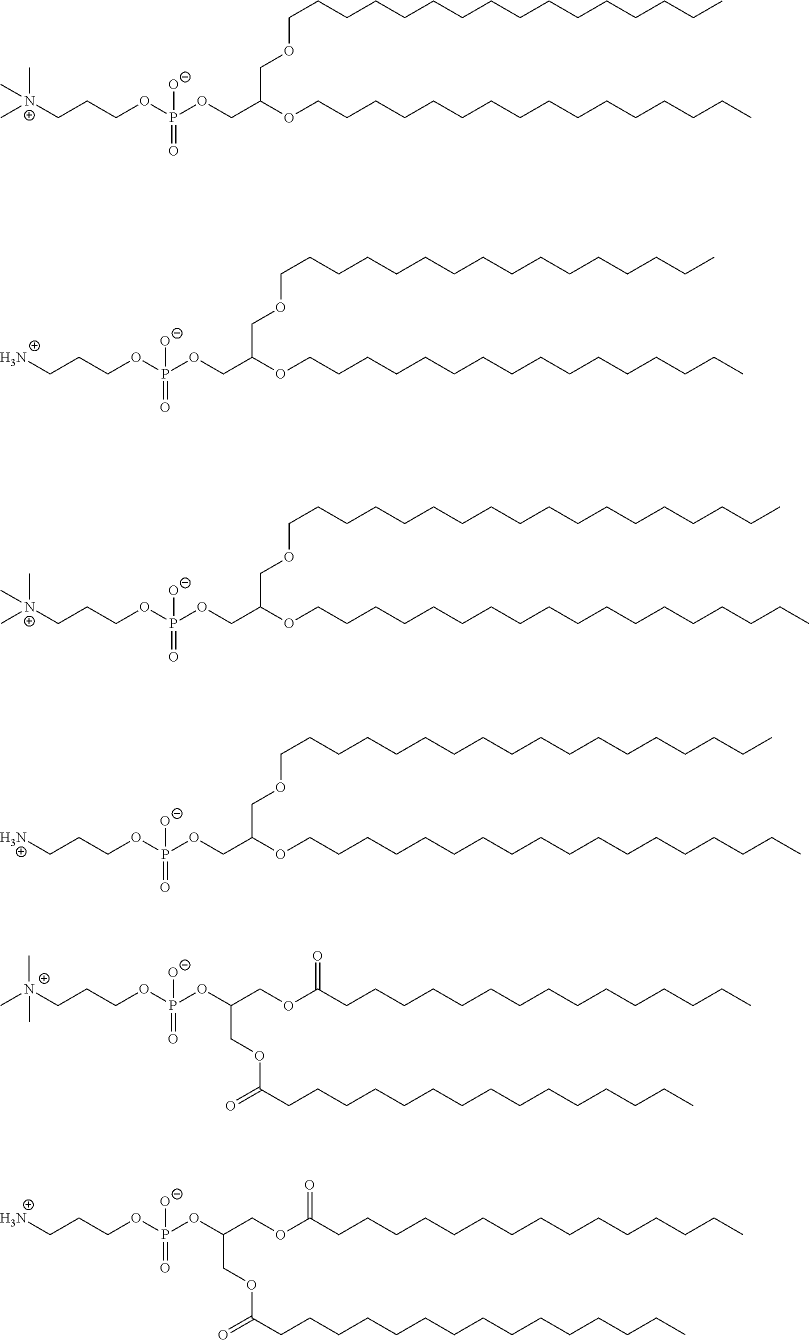

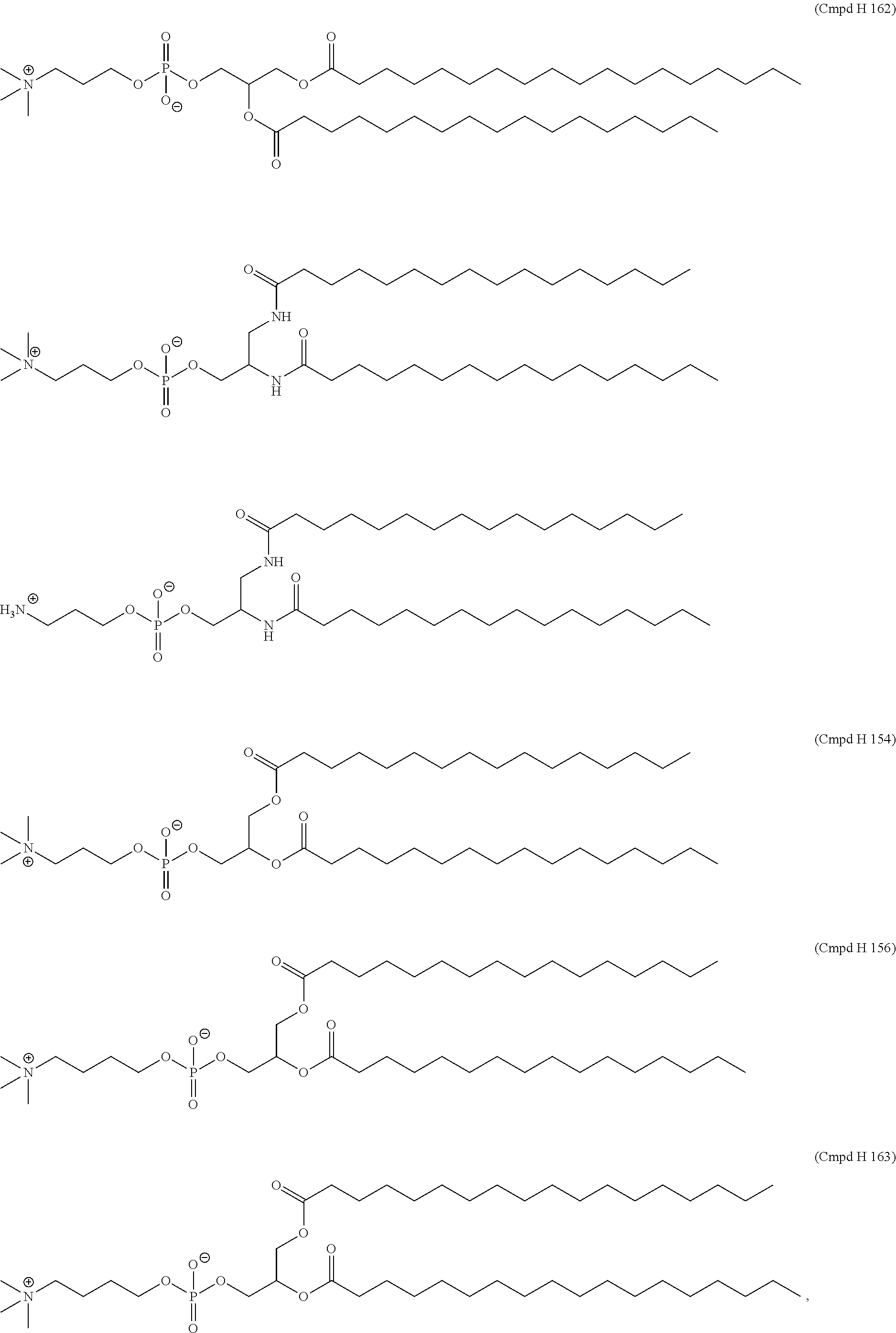

or salts thereof.

Numerous LNP formulations having phospholipids other than DSPC were prepared and tested for activity, as demonstrated in the examples below.

Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iv) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C14 to about C₂₂, preferably from about C14 to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

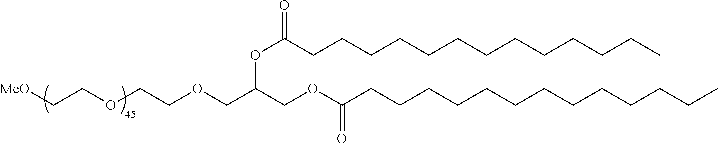

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

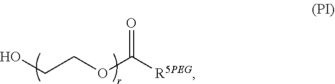

or a salt or isomer thereof, wherein:

r is an integer between 1 and 100;

R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

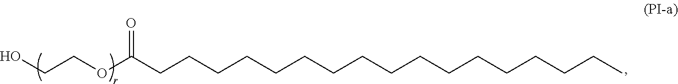

or a salt or isomer thereof, wherein r is an integer between 1 and 100.

For example, the PEG lipid is a compound of the following formula:

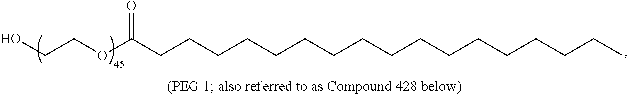

or a salt or isomer thereof.

The PEG lipid may be a compound of Formula (PII):

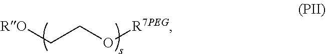

or a salt or isomer thereof, wherein:

s is an integer between 1 and 100;

R″ is a hydrogen, C₁₋₁₀ alkyl, or an oxygen protecting group;

R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR)—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

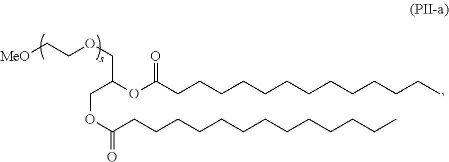

or a salt or isomer thereof, wherein s is an integer between 1 and 100.

For example, the PEG lipid is a compound of the following formula:

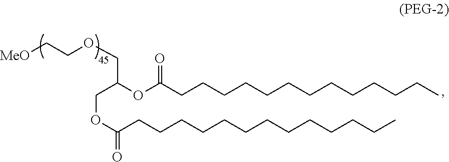

or a salt or isomer thereof.

In certain embodiments, a PEG lipid useful in the present invention is a compound of Formula (PIII). Provided herein are compounds of Formula (PIII):

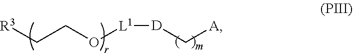

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

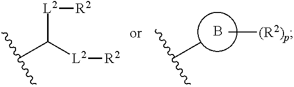

each instance of L² is independently a bond or optionally substituted C₁₋₆ alkylene, wherein one methylene unit of the optionally substituted C₁₋₆ alkylene is optionally replaced with 0, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

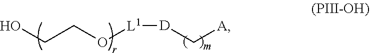

or a salt thereof.

In certain embodiments, D is a moiety obtained by click chemistry (e.g., triazole). In certain embodiments, the compound of Formula (PIII) is of Formula (PIII-a-1) or (PIII-a-2):

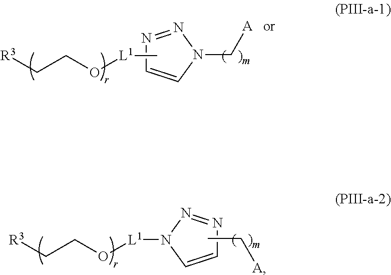

or a salt thereof.

In certain embodiments, the compound of Formula (PIII) is of one of the following formulae:

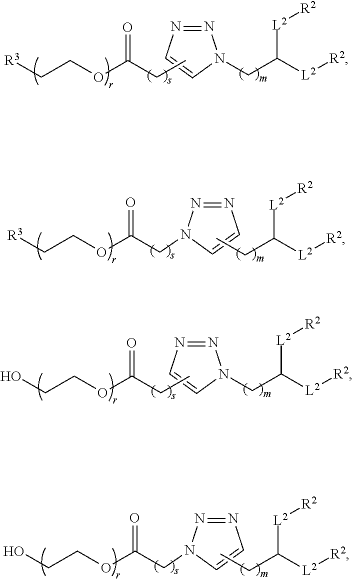

or a salt thereof, wherein

s is 0, 1, 2, 3, 4, 5, 6, 7, 8, 9, or 10.

In certain embodiments, the compound of Formula (PIII) is of one of the following formulae:

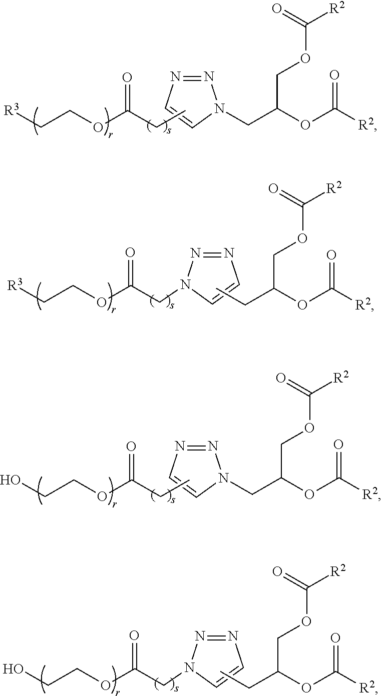

or a salt thereof.

In certain embodiments, a compound of Formula (PIII) is of one of the following formulae:

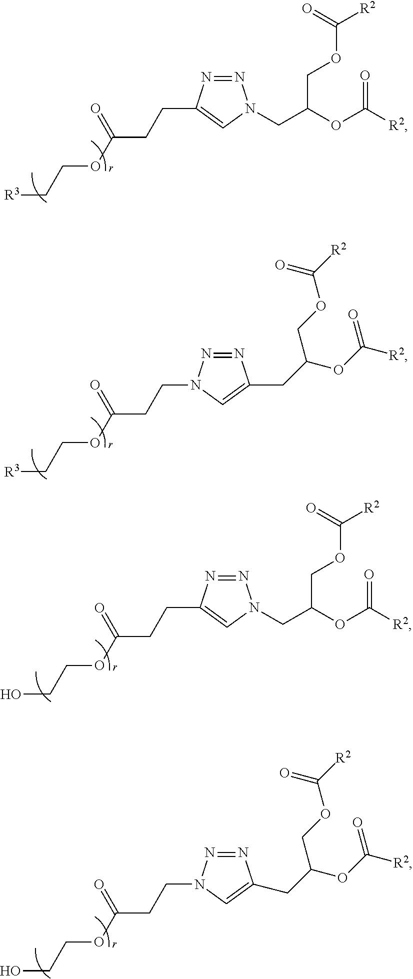

or a salt thereof.

In certain embodiments, a compound of Formula (PIII) is of one of the following formulae:

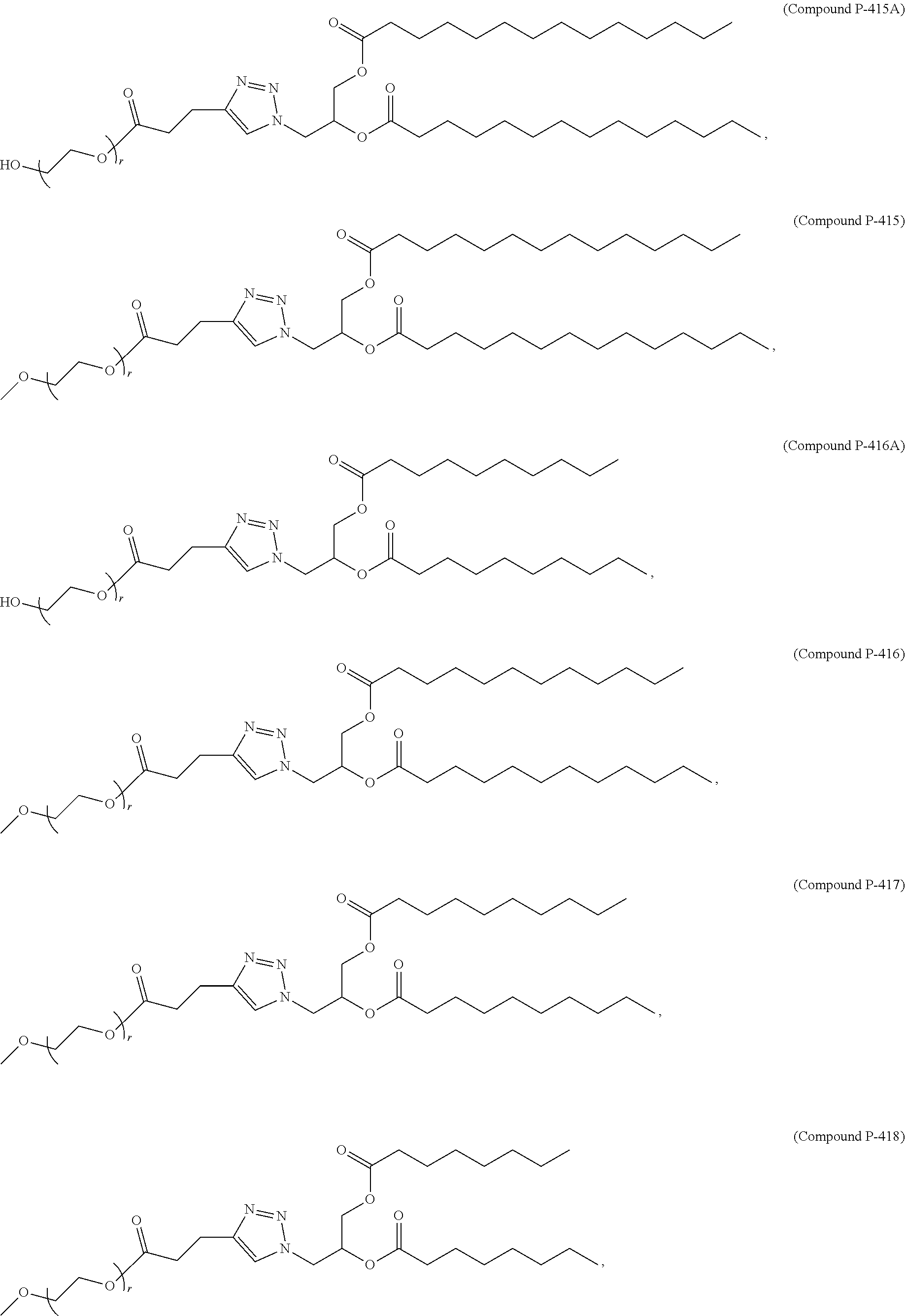

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

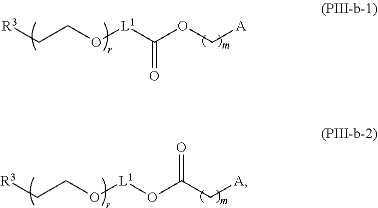

or a salt thereof.

In certain embodiments, a compound of Formula (PIII) is of Formula (PIII-b-1-OH) or (PIII-b-2-OH):

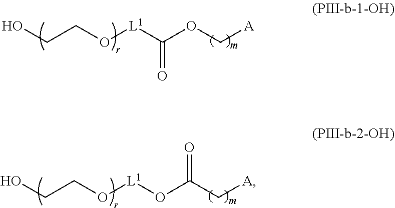

or a salt thereof.

In certain embodiments, the compound of Formula (PIII) is of one of the following formulae:

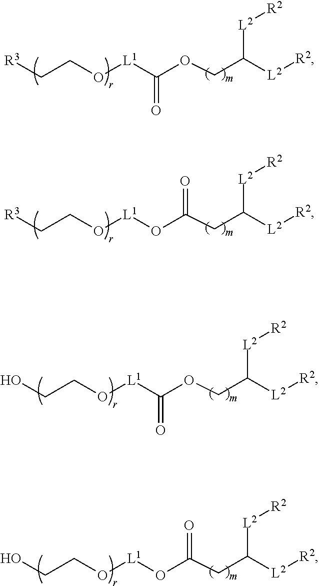

or a salt thereof.

In certain embodiments, a compound of Formula (PIII) is of one of the following formulae:

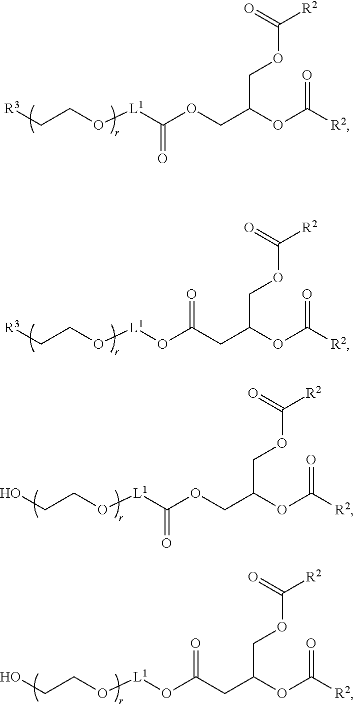

or a salt thereof.

In certain embodiments, a compound of Formula (PIII) is of one of the following formulae:

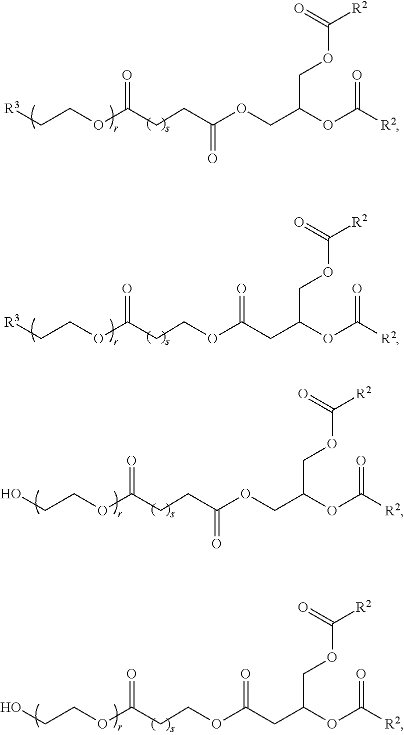

or a salt thereof.

In certain embodiments, a compound of Formula (PIII) is of one of the following formulae:

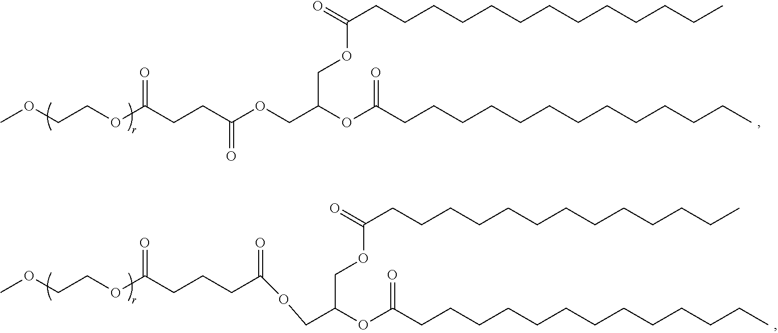

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

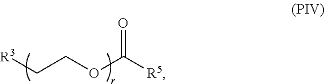

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (PIV is of Formula (PIV-OH):

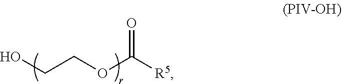

or a salt thereof. In some embodiments, r is 40-50. In some embodiments, r is 45.

In certain embodiments, a compound of Formula (PIV) is of one of the following formulae:

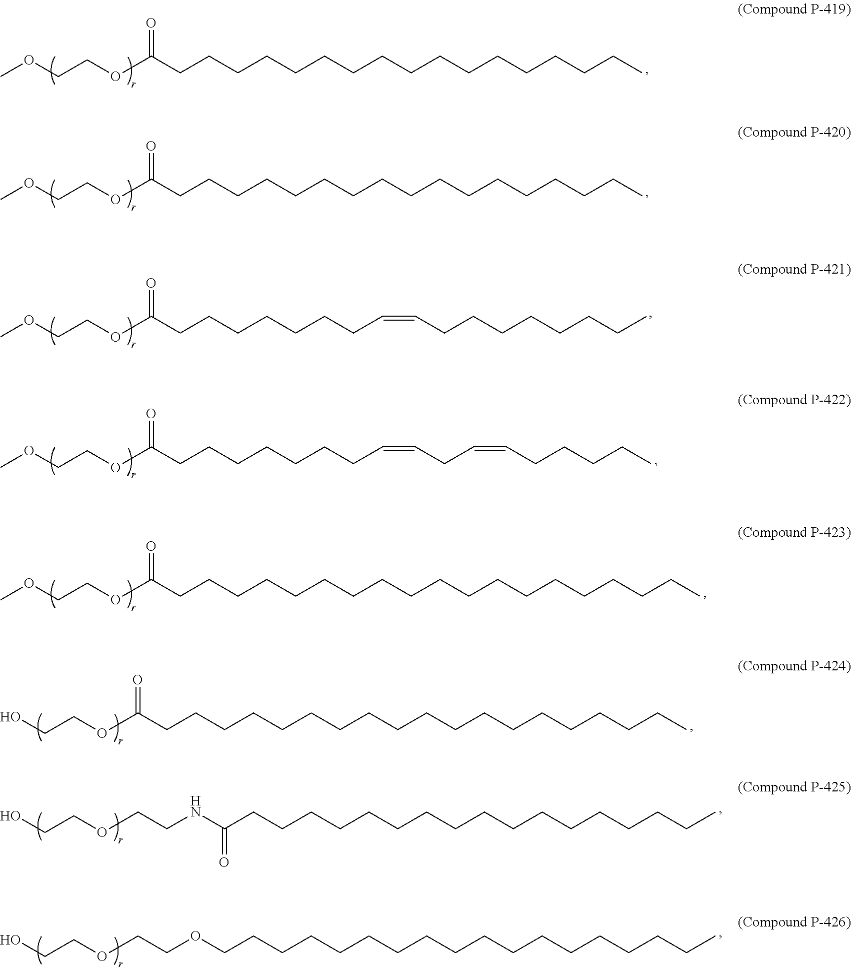

or a salt thereof. In some embodiments, r is 40-50. In some embodiments, r is 45.

In yet other embodiments the compound of Formula (PIV) is:

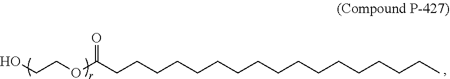

or a salt thereof.

In one embodiment, the compound of Formula (PIV) is

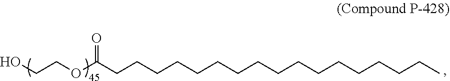

In one aspect, provided herein are lipid nanoparticles (LNPs) comprising PEG lipids of Formula (PV):

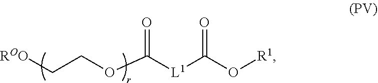

or pharmaceutically acceptable salts thereof; wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

R¹ is optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group; and

r is an integer from 2 to 100, inclusive.

In certain embodiments, the PEG lipid of Formula (PV) is of the following formula:

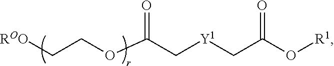

or a pharmaceutically acceptable salt thereof; wherein:

Y¹ is a bond, —CR₂—, —O—, —NR^{N}—, or —S—;

each instance of R is independently hydrogen, halogen, or optionally substituted alkyl; and

R^{N} is hydrogen, optionally substituted alkyl, optionally substituted acyl, or a nitrogen protecting group.

In certain embodiments, the PEG lipid of Formula (PV) is of one of the following formulae:

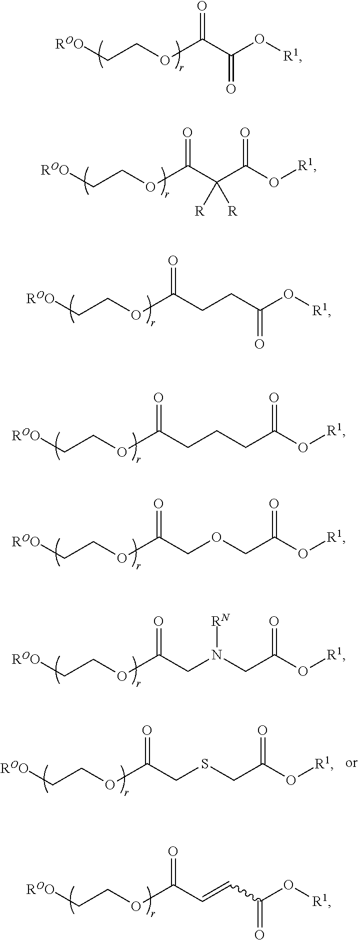

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

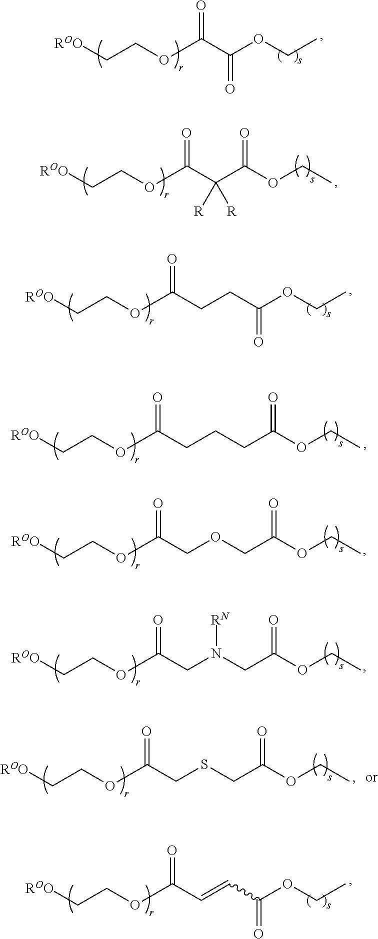

or a pharmaceutically acceptable salt thereof; wherein:

s is an integer from 5-25, inclusive.

In certain embodiments, the PEG lipid of Formula (PV) is of one of the following formulae:

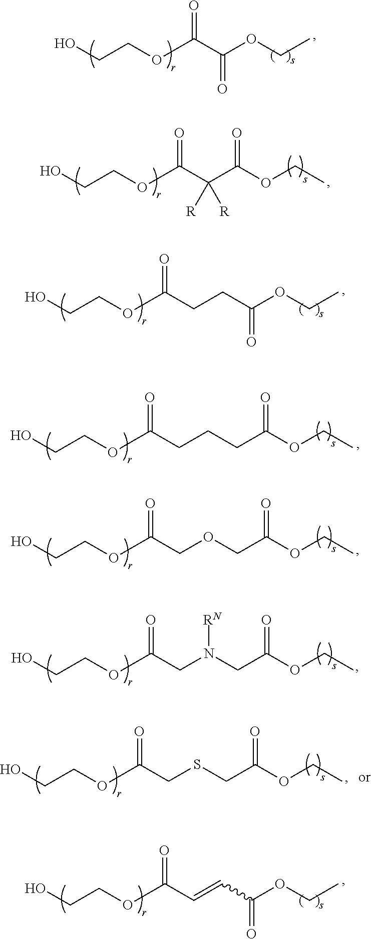

or a pharmaceutically acceptable salt thereof.

In certain embodiments, the PEG lipid of Formula (PV) is selected from the group consisting of:

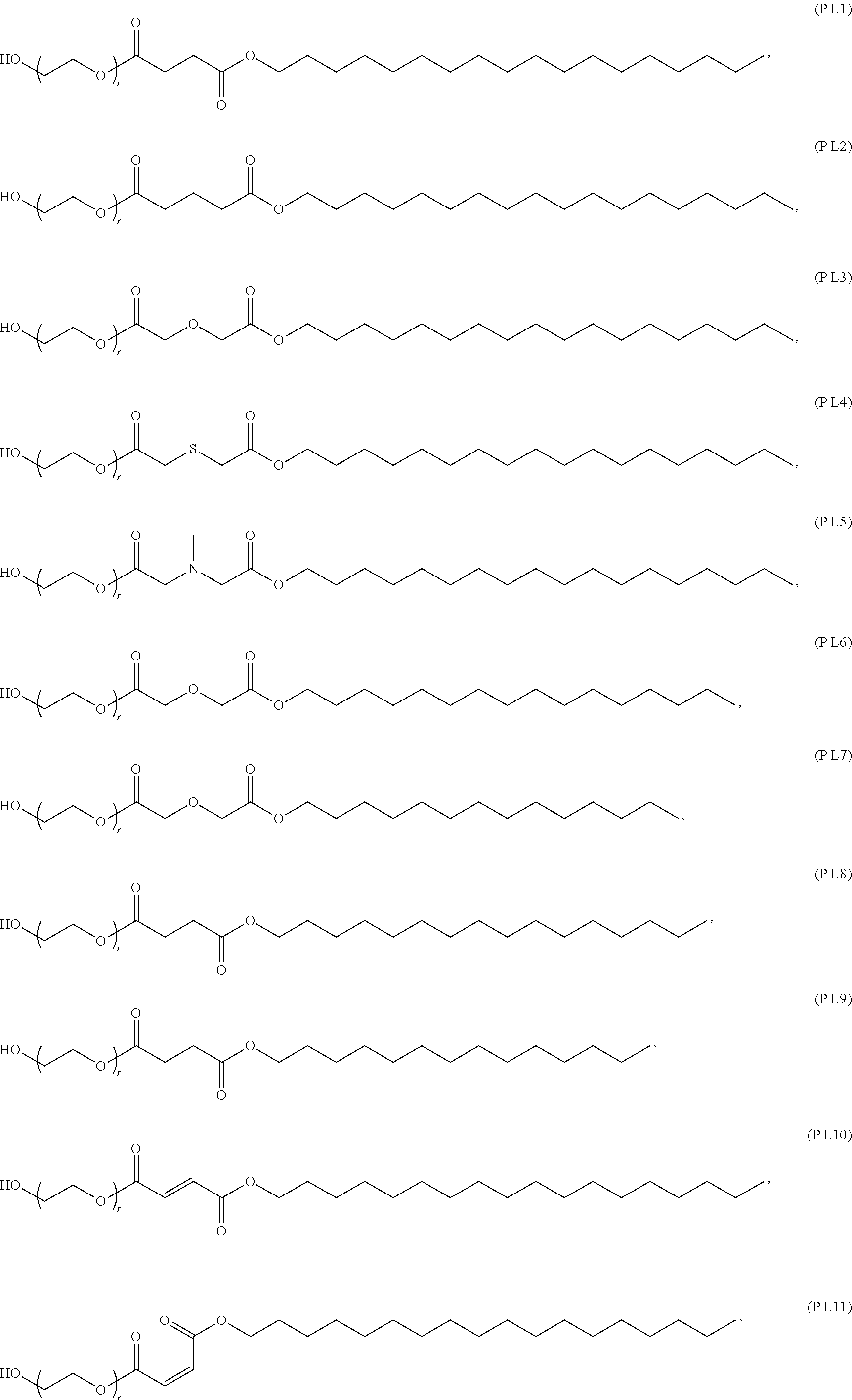

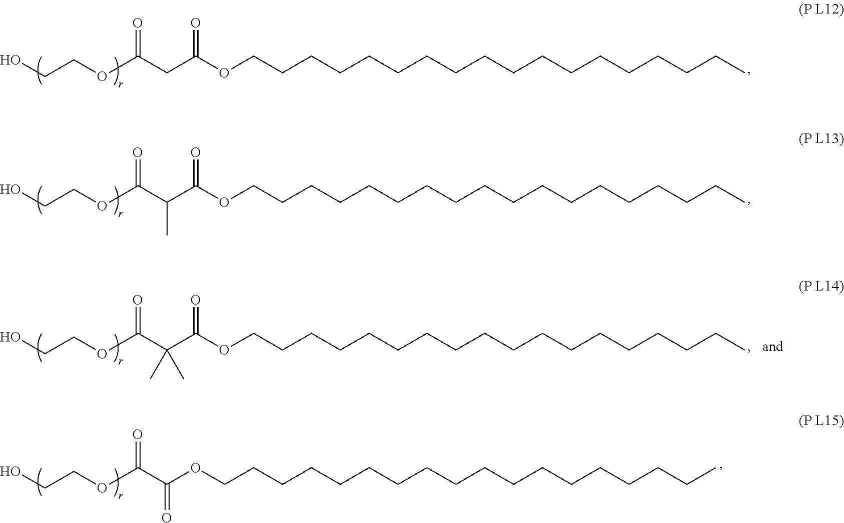

and pharmaceutically acceptable salts thereof.

In another aspect, provided herein are lipid nanoparticles (LNPs) comprising PEG lipids of Formula (PVI):

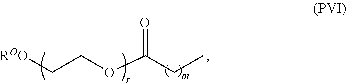

or pharmaceutically acceptable salts thereof wherein:

R^{O} is hydrogen, optionally substituted alkyl, optionally substituted acyl, or an oxygen protecting group;

r is an integer from 2 to 100, inclusive; and

m is an integer from 5-15, inclusive, or an integer from 19-30, inclusive.

In certain embodiments, the PEG lipid of Formula (PVI) is of one of the following formulae:

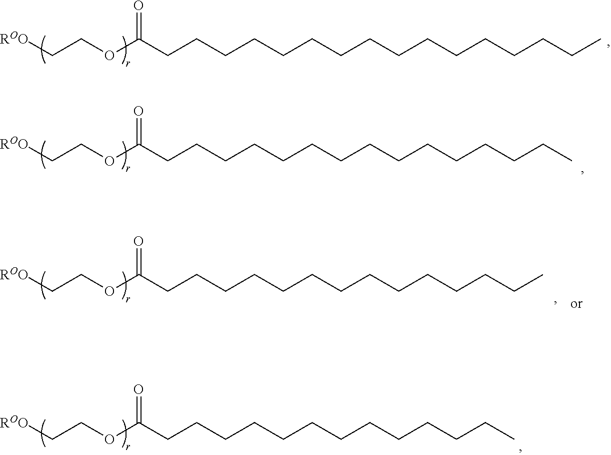

or a pharmaceutically acceptable salt thereof.

In certain embodiments, the PEG lipid of Formula (PVI) is of one of the following formulae:

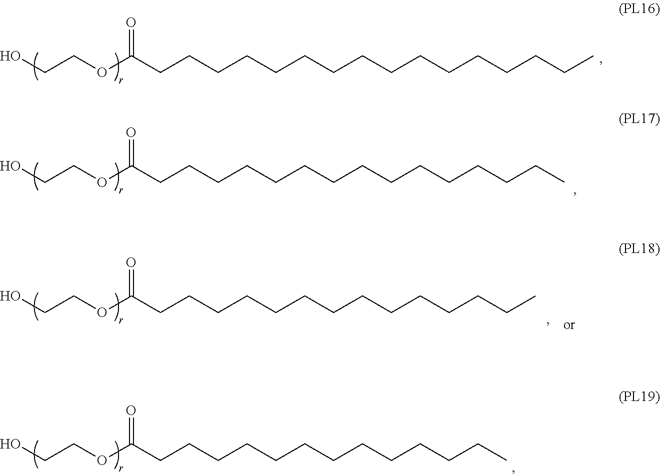

or a pharmaceutically acceptable salt thereof.

In another aspect, provided herein are lipid nanoparticles (LNPs) comprising PEG lipids of Formula (PVII):

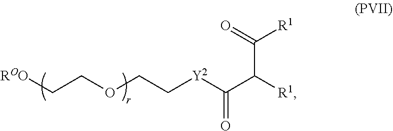

or pharmaceutically acceptable salts thereof, wherein:

Y² is —O—, —NR^{N}—, or —S—

each instance of R¹ is independently optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group;

R^{N} is hydrogen, optionally substituted alkyl, optionally substituted acyl, or a nitrogen protecting group; and

r is an integer from 2 to 100, inclusive.

In certain embodiments, the PEG lipid of Formula (PVII) is of one of the following

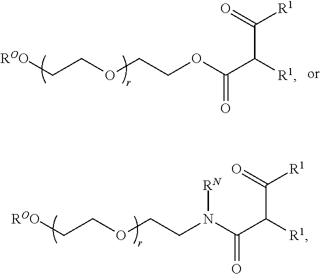

or a pharmaceutically acceptable salt thereof.

In certain embodiments, the PEG lipid of Formula (PVII) is of one of the following formulae:

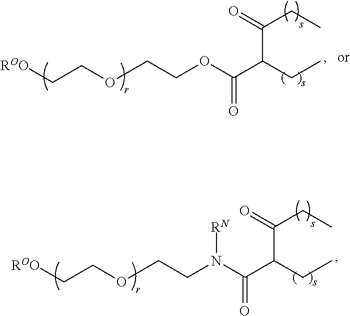

or a pharmaceutically acceptable salt thereof; wherein:

each instance of s is independently an integer from 5-25, inclusive.

In certain embodiments, the PEG lipid of Formula (PVII) is of one of the following formulae:

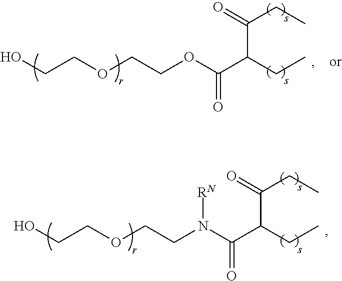

or a pharmaceutically acceptable salt thereof.

In certain embodiments, the PEG lipid of Formula (PVII) is selected from the group consisting of:

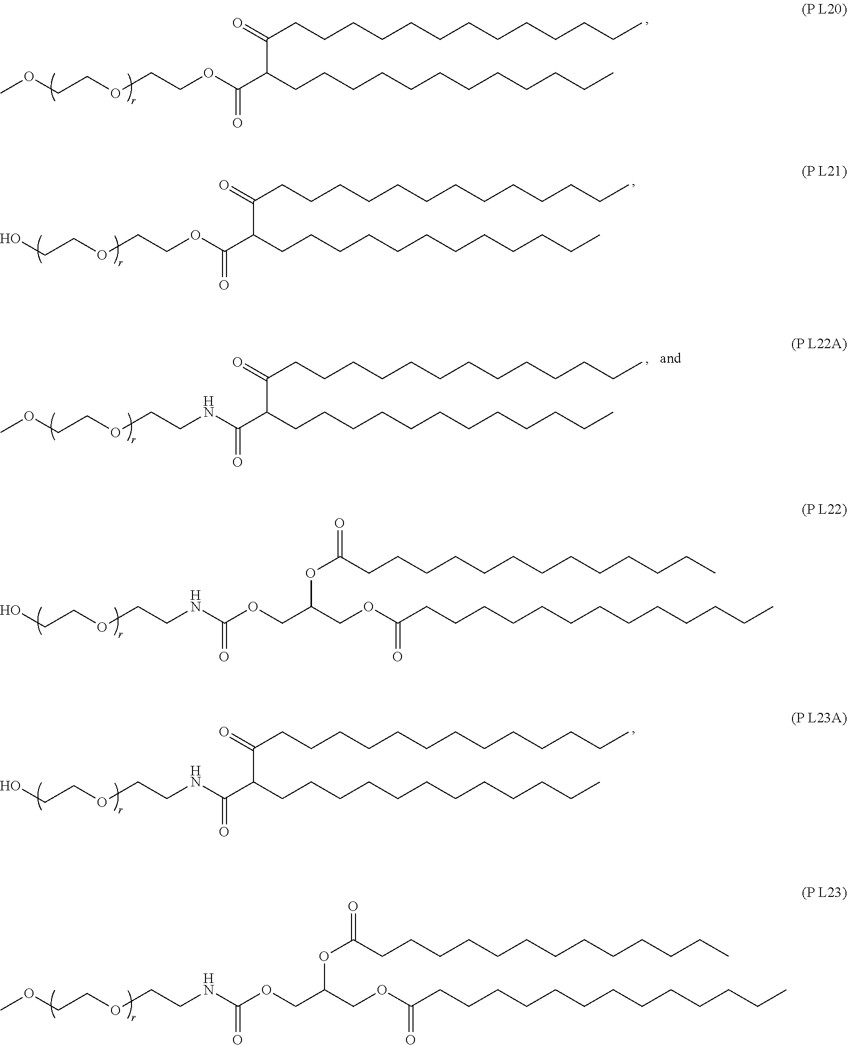

and pharmaceutically acceptable salts thereof.

In another aspect, provided herein are lipid nanoparticles (LNPs) comprising PEG lipids of Formula (PVIII):

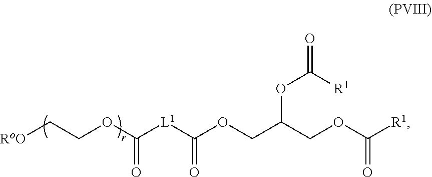

or pharmaceutically acceptable salts thereof, wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

each instance of R¹ is independently optionally substituted C₅₋₃₀ alkyl, optionally substituted C₃₋₃₀ alkenyl, or optionally substituted C₅₋₃₀ alkynyl;

R^{O} is hydrogen, optionally substituted alkyl, optionally substituted acyl, or an oxygen protecting group;

r is an integer from 2 to 100, inclusive;

provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

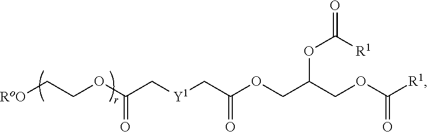

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl;

R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

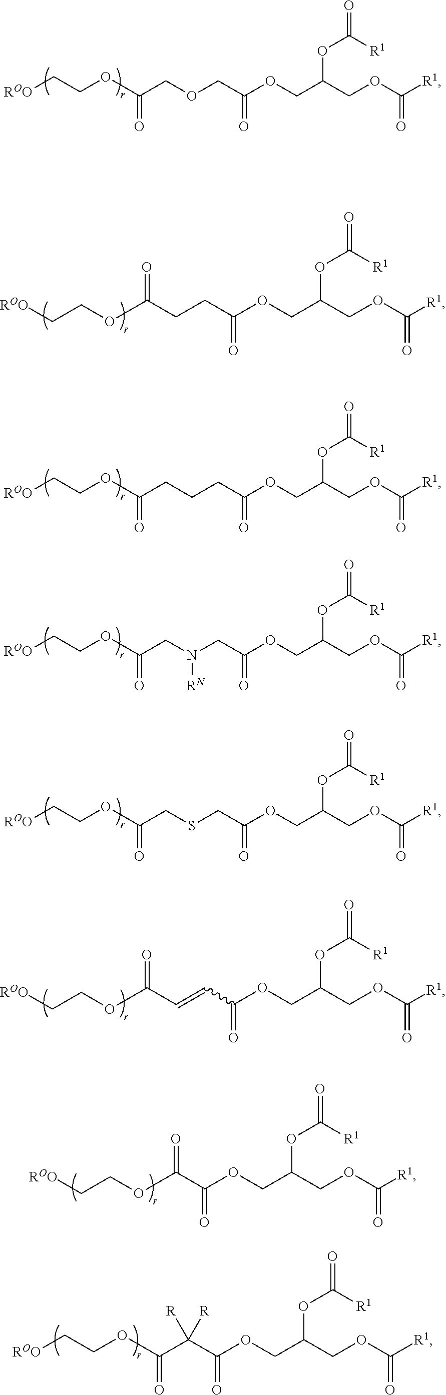

or a pharmaceutically acceptable salt thereof, wherein:
each instance of R is independently hydrogen, halogen, or optionally substituted alkyl.

In certain embodiments, the PEG lipid of Formula (PVIII) is of one of the following formulae:

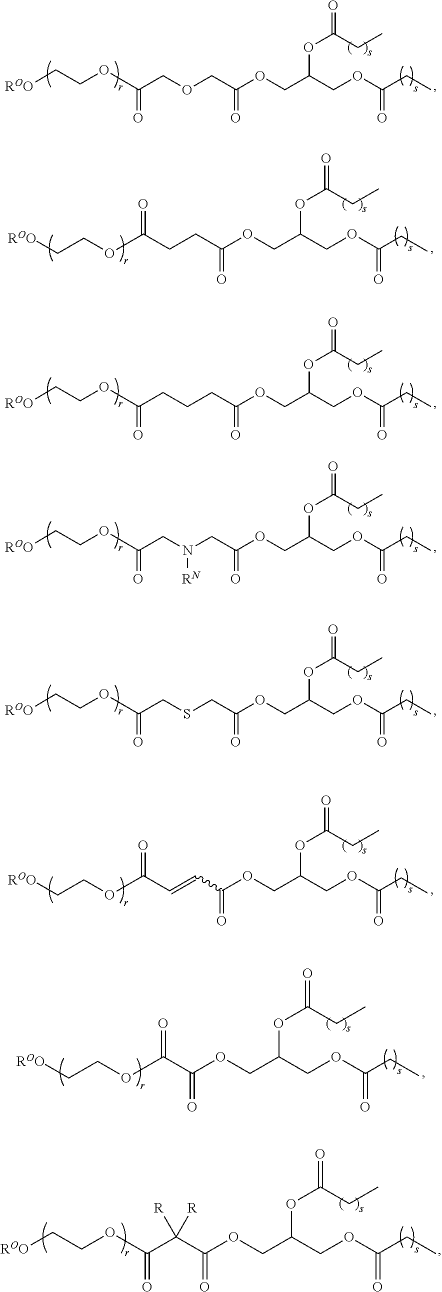

or a pharmaceutically acceptable salt thereof; wherein:

each instance of R is independently hydrogen, halogen, or optionally substituted alkyl; and

each s is independently an integer from 5-25, inclusive.

In certain embodiments, the PEG lipid of Formula (PVIII) is of one of the following formulae:

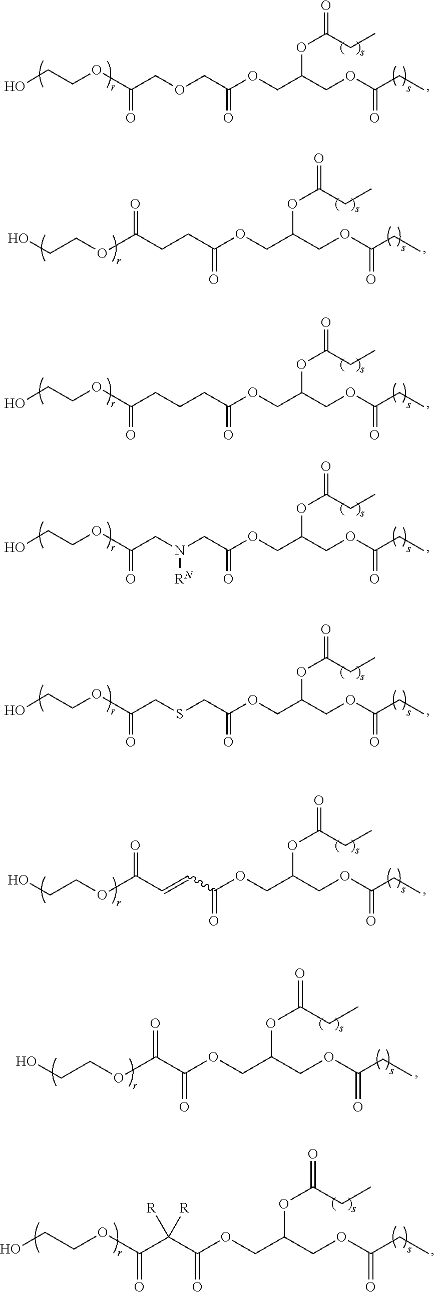

or a pharmaceutically acceptable salt thereof.

In certain embodiments, the PEG lipid of Formula (PVIII) is selected from the group consisting of:

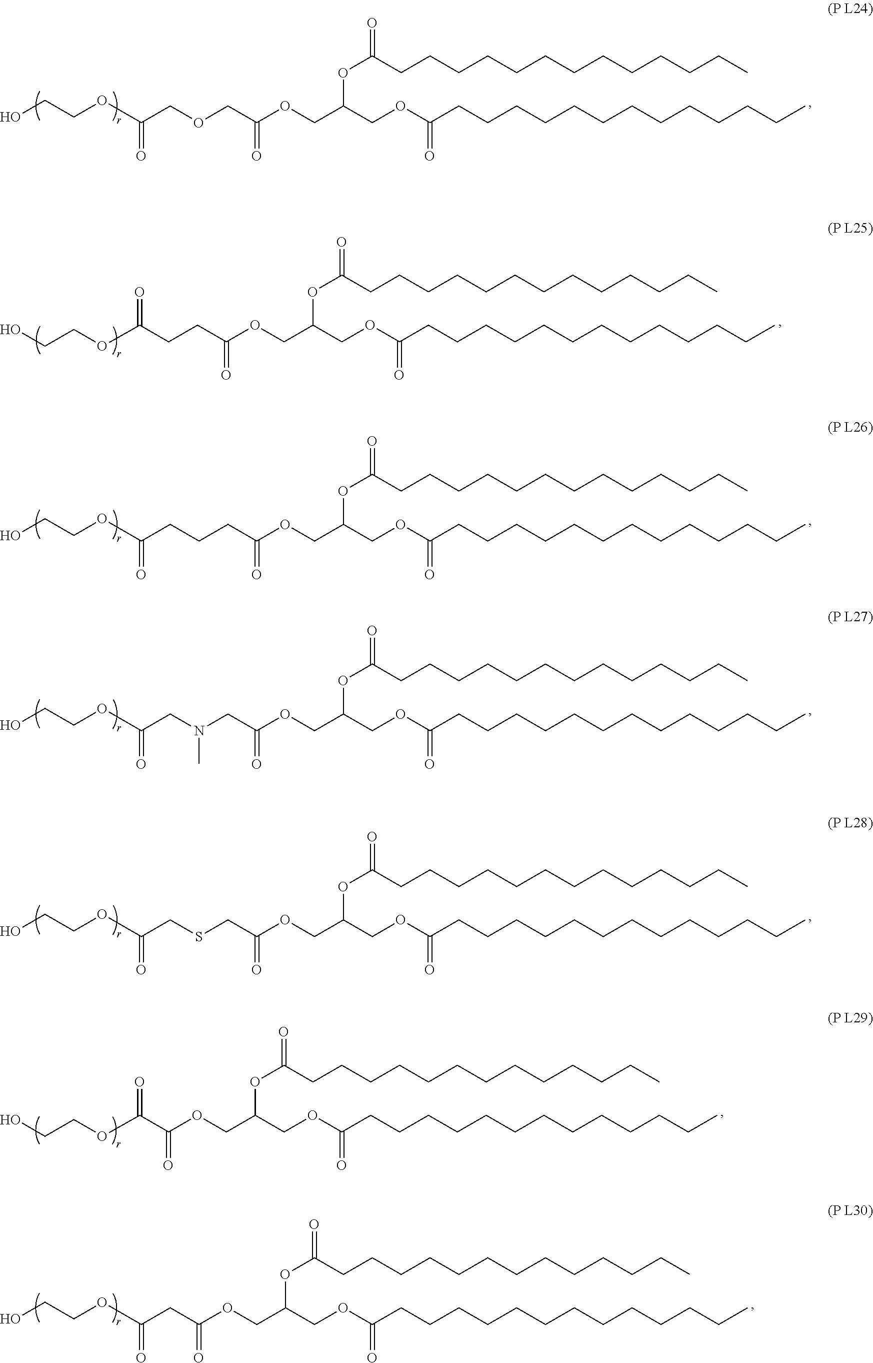

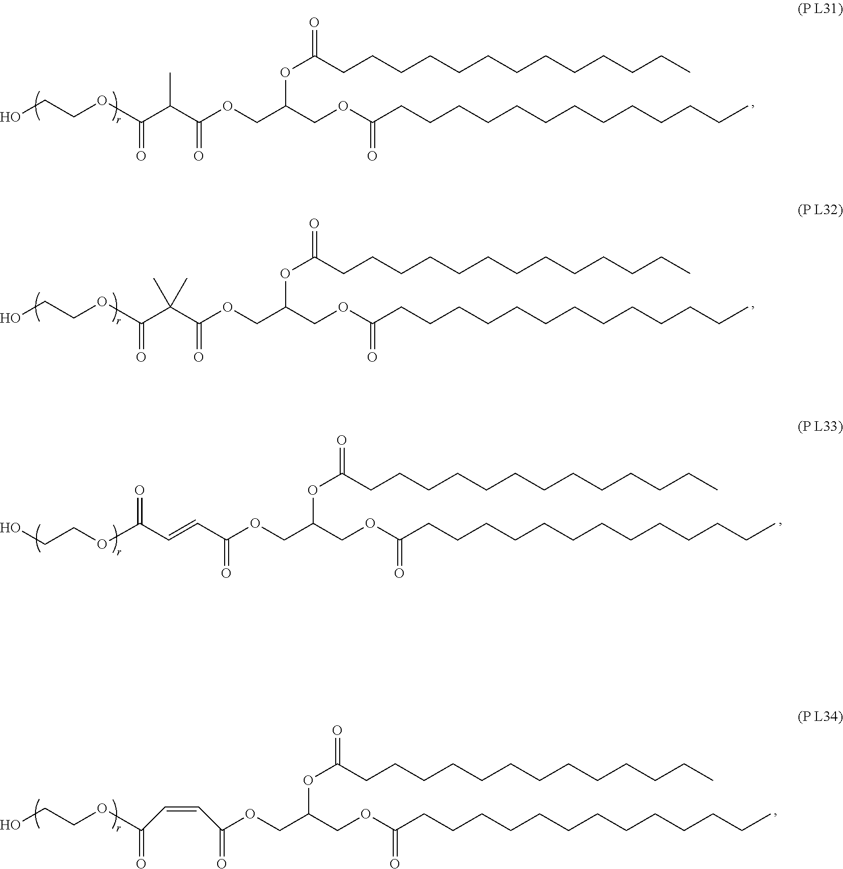

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

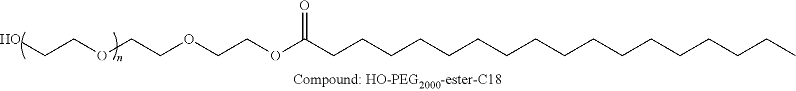

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated and backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, an immune cell delivery lipid of the pharmaceutical compositions disclosed herein does not comprise a PEG-lipid.

In one embodiment, an immune cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, an immune cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of: Cmpd 428, PL16, PL17, PL 18, PL19, PL 1, and PL 2.

Immune Cell Delivery Potentiating Lipids

An effective amount of the immune cell delivery potentiating lipid in an LNP enhances delivery of the agent to an immune cell (e.g., a human or primate immune cell) relative to an LNP lacking the immune cell delivery potentiating lipid, thereby creating an immune cell delivery LNP. Immune cell delivery potentiating lipids can be characterized in that, when present in an LNP, they promote delivery of the agent present in the LNP to immune cells as compared to a control LNP lacking the immune cell delivery potentiating lipid.

In one embodiment, the presence of at least one immune cell delivery potentiating lipid in an LNP results in an increase in the percentage of LNPs associated with immune cells as compared to a control LNP lacking at least one immune cell delivery potentiating lipid. In another embodiment, the presence of at least one immune cell delivery potentiating lipid in an LNP results in an increase in the delivery of a nucleic acid molecule agent to immune cells as compared to a control LNP lacking the immune cell delivery potentiating lipid. In one embodiment, the presence of at least one immune cell delivery potentiating lipid in an LNP results in an increase in the delivery of a nucleic acid molecule agent to B cells as compared to a control LNP lacking the immune cell delivery potentiating lipid. In particular, in one embodiment, the presence of at least one immune cell delivery potentiating lipid in an LNP results in an increase in the delivery of a nucleic acid molecule agent to myeloid cells as compared to a control LNP lacking the immune cell delivery potentiating lipid. In one embodiment, the presence of at least one immune cell delivery potentiating lipid in an LNP results in an increase in the delivery of a nucleic acid molecule agent to T cells as compared to a control LNP lacking the immune cell delivery potentiating lipid.

In one embodiment, the presence of at least one immune cell delivery potentiating lipid in an LNP results in an increase in the percentage of LNPs binding to C1q as compared to a control LNP lacking at least one immune cell delivery potentiating lipid. In one embodiment, the presence of at least one immune cell delivery potentiating lipid in an LNP results in an increase in the percentage of C1q-bound LNPs taken up by immune cells (e.g., opsonized by immune cells) as compared to a control LNP lacking at least one immune cell delivery potentiating lipid.

In one embodiment, when the nucleic acid molecule is an mRNA, the presence of at least one immune cell delivery potentiating lipid results in at least about 2-fold greater expression of a protein molecule encoded by the mRNA in immune cells (e.g., a T cells, B cells, monocytes) as compared to a control LNP lacking the immune cell delivery potentiating lipid.

In one embodiment, an immune cell delivery potentiating lipid is an ionizable lipid. In any of the foregoing or related aspects, the ionizable lipid (denoted by I) of the LNP of the disclosure comprises a compound included in any e.g. a compound having any of Formula (I I), (I IA), (I IB), (III), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, 148, I 50, I 109, I 111, I 113, I 181, I 182, I 244, I 292, I 301, I 321, I 322, I 326, I 328, I 330, I 331, I 332 or IM.

In one embodiment, an immune cell delivery potentiating lipid is an ionizable lipid. In any of the foregoing or related aspects, the ionizable lipid of the LNP of the disclosure comprises a compound described herein as Compound X, Compound Y, Compound I-321, Compound I-292, Compound I-326, Compound I-182, Compound I-301, Compound I-48, Compound I-50, Compound I-328, Compound I-330, Compound I-109, Compound I-111 or Compound I-181.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 309, I 317, I 321, I 322, I 326, I 328, I 330, I 331, I 332, I 347, I 348, I 349, I 350, I 351 and I 352. In another embodiment, the ionizable lipid of the LNP of the disclosure comprises a compound selected from the group consisting of: Compound Nos. I 18 (also referred to as Compound X), 125 (also referred to as Compound Y), 148, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

It will be understood that in embodiments where the immune cell delivery potentiating lipid comprises an ionizable lipid, it may be the only ionizable lipid present in the LNP or it may be present as a blend with at least one additional ionizable lipid. That is to say that a blend of ionizable lipids (e.g., more than one that have immune cell delivery potentiating effects or one that has an immune cell delivery potentiating effect and at least one that does not) may be employed.

In one embodiment, an immune cell delivery potentiating lipid comprises a sterol. In another embodiment, an immune cell delivery potentiating lipid comprises a naturally occurring sterol. In another embodiment, an immune cell delivery potentiating lipid comprises a modified sterol. In one embodiment, an immune cell delivery potentiating lipid comprises one or more phytosterols. In one embodiment, the immune cell delivery potentiating lipid comprises a phytosterol/cholesterol blend.

In one embodiment, the immune cell delivery potentiating lipid comprises an effective amount of a phytosterol.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

The term “effective amount of phytosterol” is intended to mean an amount of one or more phytosterols in a lipid-based composition, including an LNP, that will elicit a desired activity (e.g., enhanced delivery, enhanced immune cell uptake, enhanced nucleic acid activity). In some embodiments, an effective amount of phytosterol is all or substantially all (i.e., about 99-100%) of the sterol in a lipid nanoparticle. In some embodiments, an effective amount of phytosterol is less than all or substantially all of the sterol in a lipid nanoparticle (less than about 99-100%), but greater than the amount of non-phytosterol sterol in the lipid nanoparticle. In some embodiments, an effective amount of phytosterol is greater than 50%, greater than 60%, greater than 70%, greater than 75%, greater than 80%, greater than 85%, greater than 90% or greater than 95% the total amount of sterol in a lipid nanoparticle. In some embodiments, an effective amount of phytosterol is 95-100%, 75-100%, or 50-100% of the total amount of sterol in a lipid nanoparticle.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, A5-avenaserol, A7-avenaserol or a A7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

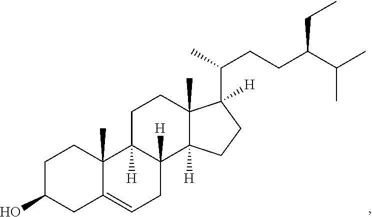

including analogs, salts or esters thereof.

In some embodiments, the sitosterol is a stigmasterol.

In some embodiments, the stigmasterol has the formula:

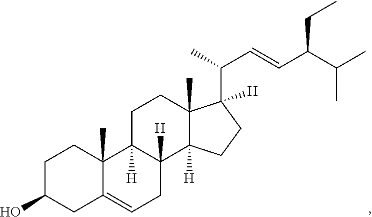

including analogs, salts or esters thereof.

In some embodiments, the sitosterol is a campesterol.

In some embodiments, the campesterol has the formula:

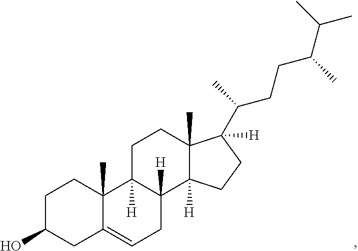

including analogs, salts or esters thereof.

In some embodiments, the sitosterol is a sitostanol.

In some embodiments, the sitostanol has the formula:

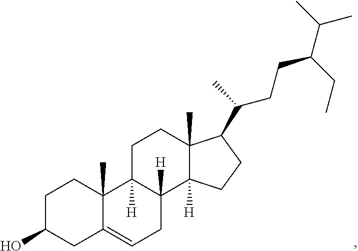

including analogs, salts or esters thereof.

In some embodiments, the sitosterol is a campestanol.

In some embodiments, the campestanol has the formula:

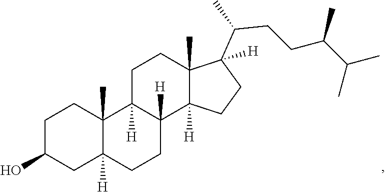

including analogs, salts or esters thereof.

In some embodiments, the sitosterol is a brassicasterol.

In some embodiments, the brassicasterol has the formula:

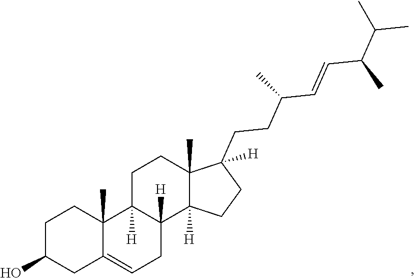

including analogs, salts or esters thereof.

In some embodiments, the sitosterol is a fucosterol.

In some embodiments, the fucosterol has the formula:

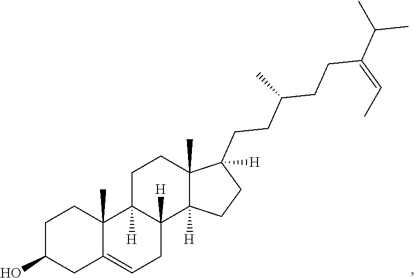

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, an immune cell delivery enhancing LNP comprises more than one type of structural lipid.

For example, in one embodiment, the immune cell delivery enhancing LNP comprises at least one immune cell delivery potentiating lipid which is a phytosterol. In one embodiment, the phytosterol is the only structural lipid present in the LNP. In another embodiment, the immune cell delivery LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g, phospholipid), 38.5% structural lipid, and 1.5% PEG lipid. In certain embodiments, the LNP comprises 50% ionizable lipid, 10% helper lipid (e.g, phospholipid), 38% structural lipid, and 2% PEG lipid. In certain embodiments, the LNP comprises 50% ionizable lipid, 20% helper lipid (e.g, phospholipid), 28.5% structural lipid, and 1.5% PEG lipid. In certain embodiments, the LNP comprises 50% ionizable lipid, 20% helper lipid (e.g, phospholipid), 28% structural lipid, and 2% PEG lipid. In certain embodiments, the LNP comprises 40% ionizable lipid, 30% helper lipid (e.g, phospholipid), 28.5% structural lipid, and 1.5% PEG lipid. In certain embodiments, the LNP comprises 40% ionizable lipid, 30% helper lipid (e.g, phospholipid), 28% structural lipid, and 2% PEG lipid. In certain embodiments, the LNP comprises 45% ionizable lipid, 20% helper lipid (e.g, phospholipid), 33.5% structural lipid, and 1.5% PEG lipid. In certain embodiments, the LNP comprises 45% ionizable lipid, 20% helper lipid (e.g, phospholipid), 33% structural lipid, and 2% PEG lipid.

In one aspect, the immune cell delivery enhancing LNP comprises phytosterol and the LNP does not comprise an additional structural lipid. Accordingly, the structural lipid (sterol) component of the LNP consists of phytosterol. In another aspect, the immune cell delivery enhancing LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition S-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, an immune cell delivery enhancing LNP comprises a non-cationic helper lipid, e.g., phospholipid. In any of the foregoing or related aspects, the non-cationic helper lipid (e.g, phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L1, P L2, P L16, P L17, P L18, P L19, P L22, P L23, DMG, DPG and DSG. In another embodiment, the PEG lipid is selected from the group consisting of Cmpd 428, PL16, PL17, PL 18, PL19, P L5, PL 1, and PL 2.

In one embodiment, an immune cell delivery potentiating lipid comprises an effective amount of a combination of an ionizable lipid and a phytosterol.

In other embodiments, the disclosure provides lipid nanoparticles comprising one or more immune cell delivery potentiating lipids, wherein the LNP comprises Compound X as the ionizable lipid, DSPC as the phospholipid, cholesterol or a cholesterol/β-sitosterol blend as the structural lipid and Compound 428 as the PEG lipid. In various embodiments of these Compound X-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; For the structural lipid component, in one embodiment the structural lipid is entirely cholesterol at 38% or 28%. In another embodiment, the structural lipid is cholesterol/3-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol. In another embodiment, the structural lipid is cholesterol/3-sitosterol at a total percentage of 38.5%, wherein the blend can comprise, for example: (i) 20% cholesterol and 18.5% β-sitosterol; or (ii) 10% cholesterol and 28.5% β-sitosterol.

In other embodiments, the disclosure provides lipid nanoparticles comprising one or more immune cell delivery potentiating lipids, wherein the LNP comprises Compound Y as the ionizable lipid, DSPC as the phospholipid, cholesterol or a cholesterol/β-sitosterol blend as the structural lipid and Compound 428 as the PEG lipid. In various embodiments of these Compound Y-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/3-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-182 as the ionizable lipid, DSPC as the phospholipid, cholesterol or a cholesterol/β-sitosterol blend as the structural lipid and Compound 428 as the PEG lipid. In various embodiments of these Compound I-182-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-321 as the ionizable lipid, DSPC as the phospholipid, cholesterol or a cholesterol/β-sitosterol blend as the structural lipid and Compound 428 as the PEG lipid. In various embodiments of these Compound I-321-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-292 as the ionizable lipid, DSPC as the phospholipid, cholesterol or a cholesterol/β-sitosterol blend as the structural lipid and Compound 428 as the PEG lipid. In various embodiments of these Compound 1-292-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-326 as the ionizable lipid, DSPC as the phospholipid, cholesterol or a cholesterol/β-sitosterol blend as the structural lipid and Compound 428 as the PEG lipid. In various embodiments of these Compound I-326-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-301 as the ionizable lipid, DSPC as the phospholipid, cholesterol or a cholesterol/β-sitosterol blend as the structural lipid and Compound 428 as the PEG lipid. In various embodiments of these Compound I-301-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-48 as the ionizable lipid, DSPC as the phospholipid, cholesterol or a cholesterol/β-sitosterol blend as the structural lipid and Compound 428 as the PEG lipid. In various embodiments of these Compound I-48-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-50 as the ionizable lipid, DSPC as the phospholipid, cholesterol or a cholesterol/β-sitosterol blend as the structural lipid and Compound 428 as the PEG lipid. In various embodiments of these Compound I-50-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-328 as the ionizable lipid, DSPC as the phospholipid, cholesterol or a cholesterol/β-sitosterol blend as the structural lipid and Compound 428 as the PEG lipid. In various embodiments of these Compound I-328-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-330 as the ionizable lipid, DSPC as the phospholipid, cholesterol or a cholesterol/β-sitosterol blend as the structural lipid and Compound 428 as the PEG lipid. In various embodiments of these Compound I-330-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-109 as the ionizable lipid, DSPC as the phospholipid, cholesterol or a cholesterol/β-sitosterol blend as the structural lipid and Compound 428 as the PEG lipid. In various embodiments of these Compound I-109-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-111 as the ionizable lipid, DSPC as the phospholipid, cholesterol or a cholesterol/β-sitosterol blend as the structural lipid and Compound 428 as the PEG lipid. In various embodiments of these Compound I-111-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-181 as the ionizable lipid, DSPC as the phospholipid, cholesterol or a cholesterol/β-sitosterol blend as the structural lipid and Compound 428 as the PEG lipid. In various embodiments of these Compound I-181-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/3-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises any of Compounds X, Y, I-321, I-292, I-326, I-182, I-301, I-48, I-50, I-328, I-330, I-109, I-111 or I-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesterol/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% β-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% β-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% β-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiments, the disclosure provides lipid nanoparticles comprising one or more immune cell delivery potentiating lipids, wherein the LNP comprises Compound I-301, Compound I-321 or Compound I-326 as the ionizable lipid; DSPC as the phospholipid; cholesterol or a cholesterol/β-sitosterol blend as the structural lipid; and Compound 428 as the PEG lipid. In one embodiment, the LNP enhances delivery to T cells (e.g., CD3+ T cells).

In other embodiment, the disclosure provides lipid nanoparticles comprising one or more immune cell delivery potentiating lipids, wherein the LNP comprises Compound X, Compound I-109, Compound I-111, Compound I-181, Compound I-182 or Compound I-244, wherein the LNP enhances delivery to monocytes. The other components of the LNP can be selected from those disclosed herein, for example DSPC as the phospholipid; cholesterol or a cholesterol/3-sitosterol blend as the structural lipid; and Compound 428 as the PEG lipid.

In other embodiment, the disclosure provides lipid nanoparticles comprising one or more immune cell delivery potentiating lipids, wherein the LNP comprises camposterol, β-sitostanol or stigmasterol as the structural lipid, wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; DSPC as the phospholipid; and Compound 428 as the PEG lipid.

In other embodiment, the disclosure provides lipid nanoparticles comprising one or more immune cell delivery potentiating lipids, wherein the LNP comprises DOPC, DMPE or H-409 as the helper lipid (e.g., phospholipid), wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; cholesterol, a cholesterol/β-sitosterol blend, camposterol, β-sitostanol or stigmasterol as the structural lipid; and Compound 428 as the PEG lipid.

### Exemplary Additional LNP Components

Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

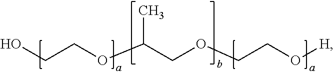

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

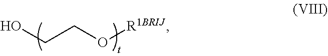

or a salt or isomer thereof, wherein:

t is an integer between 1 and 100;

R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

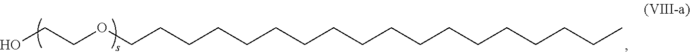

or a salt or isomer thereof.

In some embodiments, R^{1BRIJ} is C₁₈ alkenyl. For example, the polyethylene glycol ether is a compound of Formula (VIII-b):

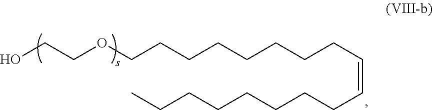

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL@.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### LNP Compositions

A lipid nanoparticle described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The LNPs of the invention comprise at least one immune cell delivery potentiating lipid. The subject LNPs comprise: an effective amount of an immune cell delivery potentiating lipid as a component of an LNP, wherein the LNP comprises an (i) ionizable lipid; (ii) cholesterol or other structural lipid; (iii) a non-cationic helper lipid or phospholipid; a (iv) PEG lipid and (v) an agent (e.g, an nucleic acid molecule) encapsulated in and/or associated with the LNP, wherein the effective amount of the immune cell delivery potentiating lipid enhances delivery of the agent to an immune cell (e.g., a human or primate immune cell) relative to an LNP lacking the immune cell delivery potentiating lipid.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol % ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol % ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol % ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol % ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

Lipid nanoparticles of the disclosure may be designed for one or more specific applications or targets. For example, the subject lipid nanoparticles may optionally be designed to further enhance delivery of a nucleic acid molecule, such as an RNA, to a particular immune cell (e.g., lymphoid cell or myeloid cell), tissue, organ, or system or group thereof in a mammal's, e.g., a human's body. Physiochemical properties of lipid nanoparticles may be altered in order to increase selectivity for particular bodily targets. For instance, particle sizes may be adjusted to promote immune cell uptake. As set forth above, the nucleic acid molecule included in a lipid nanoparticle may also be selected based on the desired delivery to immune cells. For example, a nucleic acid molecule may be selected for a particular indication, condition, disease, or disorder and/or for delivery to a particular cell, tissue, organ, or system or group thereof (e.g., localized or specific delivery).

In certain embodiments, a lipid nanoparticle may include an mRNA encoding a polypeptide of interest capable of being translated within a cell to produce a polypeptide of interest. In other embodiments, the lipid nanoparticle can include other types of agents, such as other nucleic acid agents, including DNA and/or RNA agents, as described herein, e.g., siRNAs, miRNAs, antisense nucleic acid and the like as described in further detail below.

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of a nucleic acid molecule and other elements (e.g., lipids) in a lipid nanoparticle may also vary. In some embodiments, the wt/wt ratio of the ionizable lipid component to a nucleic acid molecule, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to a nucleic acid molecule may be from about 10:1 to about 40:1. In certain embodiments, the wt/wt ratio is about 20:1. The amount of a nucleic acid molecule in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Exemplary Agents

Agents to be Delivered

The immune cell delivery lipids, and LNPs containing them, of the disclosure can be used to deliver a wide variety of different agents to immune cells (e.g., T cells, B cells, dendritic cells, myeloid cells, macrophage/monocytes) through association with, e.g., encapsulation of the agent. Typically the agent delivered by the LNP is a nucleic acid, although non-nucleic acid agents, such as small molecules, chemotherapy drugs, peptides, proteins and other biological molecules are also encompassed by the disclosure. Nucleic acids that can be delivered include DNA-based molecules (i.e., comprising deoxyribonucleotides) and RNA-based molecules (i.e., comprising ribonucleotides). Furthermore, the nucleic acid can be a naturally occurring form of the molecule or a chemically-modified form of the molecule (i.e., comprising one or more modified nucleotides).

Agents for Enhancing Protein Expression

In one embodiment, the agent associated with/encapsulated by the lipid-based composition (e.g., LNP) is an agent that enhances (i.e., increases, stimulates, upregulates) protein expression. In one embodiment, the agent increases protein expression in the target immune cell (e.g., T cell, B cell, dendritic cell, myeloid cell) to which the lipid-based composition is delivered. Additionally or alternatively, in another embodiment, the agent results in increased protein expression in other cells, e.g., bystander cells, than the target immune cell (e.g., T cell, B cell, dendritic cell, myeloid cell) to which the lipid-based composition is delivered. Non-limiting examples of types of agents that can be used for enhancing protein expression include RNAs, mRNAs, dsRNAs, CRISPR/Cas9 technology, ssDNAs and DNAs (e.g., expression vectors).

DNA Agents

In one embodiment, the agent associated with/encapsulated by the LNP is a DNA agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA agent associated with/encapsulated by the LNP can be a DNA molecule that is capable of transferring a gene into a cell, e.g., that encodes and can express a transcript. For example, the DNA agent can encode a protein of interest, to thereby increase expression of the protein of interest in an immune cell upon delivery into the immune cell by the LNP. In some embodiments, the DNA molecule can be naturally-derived, e.g., isolated from a natural source. In other embodiments, the DNA molecule is a synthetic molecule, e.g., a synthetic DNA molecule produced in vitro. In some embodiments, the DNA molecule is a recombinant molecule. Non-limiting exemplary DNA agents include plasmid expression vectors and viral expression vectors.

The DNA agents described herein, e.g., DNA vectors, can include a variety of different features. The DNA agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

RNA Agents

In one embodiment, the agent associated with/encapsulated by the LNP is an RNA agent. The RNA molecule can be a single-stranded RNA, a double-stranded RNA (dsRNA) or a molecule that is a partially double-stranded RNA, i.e., has a portion that is double-stranded and a portion that is single-stranded. The RNA molecule can be a circular RNA molecule or a linear RNA molecule.

An RNA agent associated with/encapsulated by the LNP can be an RNA agent that is capable of transferring a gene into a cell, e.g., encodes a protein of interest, to thereby increase expression of the protein of interest in an immune cell upon delivery into the immune cell by the LNP. In some embodiments, the RNA molecule can be naturally-derived, e.g., isolated from a natural source. In other embodiments, the RNA molecule is a synthetic molecule, e.g., a synthetic RNA molecule produced in vitro.

Non-limiting examples of RNA agents include messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and CRISPR/Cas9 technology, each of which is described further in subsections below.

Messenger RNA (mRNA)

In some embodiments, the disclosure provides a lipid composition (e.g., lipid nanoparticle) comprising at least one mRNA, for use in the methods described herein.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 60. An exemplary 3′ UTR for use in the constructs is shown in SEQ ID NO: 61. An exemplary 3′ UTR comprising miR-122 and/or miR-142-3p binding sites for use in the constructs is shown in SEQ ID NO: 62. In one embodiment, hepatocyte expression is reduced by including miR122 binding sites. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 63), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 63) fragments or variants thereof. One example of a polynucleotide sequence encoding the 2A peptide is: GGAAGCGGAGCTACTAACTTCAGCCTGCTGAAGCAGGCTGGAGACGTGGAGGAGAA CCCTGGACCT (SEQ ID NO: 64). In one illustrative embodiment, a 2A peptide is encoded by the following sequence: 5′-TCCGGACTCAGATCCGGGGATCTCAAAATTGTCGCTCCTGTCAAACAAACTCTTAAC TTTGATTTACTCAAACTGGCTGGGGATGTAGAAAGCAATCCAGGTCCACTC-3′ (SEQ ID NO: 65). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine, 5-methyl-uridine (m5U, i.e., having the nucleobase deoxythymine), 1-methyl-pseudouridine (m1 ψ), 5-methyl-2-thio-uridine (m5 s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (4′m), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, ca-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, O6-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.) In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 17 and Table 18. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 17
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 18
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75%
UTP
25% 5-Aminoallyl-CTP + 75% CTP/75% 5-Methoxy-UTP + 25%
UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP + 75%
UTP
25% 5-Hydroxymethyl-CTP + 75% CTP/75% 5-Methoxy-UTP + 25%
UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-
pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-
Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-
Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75%
UTP
25% 5-Trifluoromethyl-CTP + 75% CTP/75% 5-Methoxy-UTP + 25%
UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-
Methyl-pseudo-UTP
50% 5-Methyl-CTP + 50% CTP/25% 5-Methoxy-UTP + 75%
UTP
50% 5-Methyl-CTP + 50% CTP/50% 5-Methoxy-UTP + 50% 1-
Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-
Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxymethyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoromethyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75%
UTP
75% 5-Aminoallyl-CTP + 25% CTP/75% 5-Methoxy-UTP + 25%
UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75%
UTP
75% 5-Hydroxymethyl-CTP + 25% CTP/75% 5-Methoxy-UTP + 25%
UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-
Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-
Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-
Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75%
UTP
75% 5-Trifluoromethyl-CTP + 25% CTP/75% 5-Methoxy-UTP + 25%
UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 17 or Table 18.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.” Non-limiting examples of sensor sequences are described in U.S. Publication 2014/0200261, the contents of which are incorporated herein by reference in their entirety.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR can be included in a nucleic acid molecule (e.g., an RNA, e.g., mRNA) to minimize expression in cell types other than lymphoid cells. In one embodiment, miR122 can be used. In another embodiment, miR126 can be used. In still another embodiment, multiple copies of these miRs or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

In one embodiment, a nucleic acid molecule (e.g., RNA, e.g., mRNA) of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a nucleic acid molecule (e.g., RNA, e.g., mRNA) of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a nucleic acid molecule (e.g., RNA, e.g., mRNA) of the disclosure to regulate expression of the nucleic acid molecule (e.g., RNA, e.g., mRNA) in the liver. Liver specific miRNA binding sites can be engineered alone or further in combination with immune cell (e.g., APC) miRNA binding sites in a nucleic acid molecule (e.g., RNA, e.g., mRNA) of the disclosure. In one embodiment, miRNA binding sites that promote degradation of mRNAs by hepatocytes are present in an mRNA molecule agent.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a nucleic acid molecule (e.g., RNA, e.g., mRNA) of the disclosure to regulate expression of the nucleic acid molecule (e.g., RNA, e.g., mRNA) in the lung. Lung specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a nucleic acid molecule (e.g., RNA, e.g., mRNA) of the disclosure to regulate expression of the nucleic acid molecule (e.g., RNA, e.g., mRNA) in the heart. Heart specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a nucleic acid molecule (e.g., RNA, e.g., mRNA) of the disclosure to regulate expression of the nucleic acid molecule (e.g., RNA, e.g., mRNA) in the nervous system. Nervous system specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a nucleic acid molecule (e.g., RNA, e.g., mRNA) of the disclosure to regulate expression of the nucleic acid molecule (e.g., RNA, e.g., mRNA) in the pancreas. Pancreas specific miRNA binding sites can be engineered alone or further in combination with immune cell (e.g. APC) miRNA binding sites in a nucleic acid molecule (e.g., RNA, e.g., mRNA) of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a nucleic acid molecule (e.g., RNA, e.g., mRNA) of the disclosure to regulate expression of the nucleic acid molecule (e.g., RNA, e.g., mRNA) in the kidney. Kidney specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. miRNA binding sites from any muscle specific miRNA can be introduced to or removed from a nucleic acid molecule (e.g., RNA, e.g., mRNA) of the disclosure to regulate expression of the nucleic acid molecule (e.g., RNA, e.g., mRNA) in the muscle. Muscle specific miRNA binding sites can be engineered alone or further in combination with immune cell (e.g., APC) miRNA binding sites in a nucleic acid molecule (e.g., RNA, e.g., mRNA) of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, the binding sites of embryonic stem cell specific miRNAs can be included in or removed from the 3′UTR of a nucleic acid molecule (e.g., RNA, e.g., mRNA) of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3′UTR of a nucleic acid molecule (e.g., RNA, e.g., mRNA) of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the nucleic acid molecules (e.g., RNA, e.g., mRNA) of the disclosure, miRNA binding sites that are involved in such processes can be removed or introduced, in order to tailor the expression of the nucleic acid molecules (e.g., RNA, e.g., mRNA) to biologically relevant cell types or relevant biological processes. In this context, the nucleic acid molecules (e.g., RNA, e.g., mRNA) of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into a nucleic acid molecule (e.g., RNA, e.g., mRNA) encoding the polypeptide. In one example, a nucleic acid molecule (e.g., RNA, e.g., mRNA) may include one or more miRNA binding sites that are bound by miRNAs that have higher expression in one tissue type as compared to another. In another example, a nucleic acid molecule (e.g., RNA, e.g., mRNA) may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the nucleic acid molecule (e.g., RNA, e.g., mRNA) typically will show increased expression.

Liver cancer cells (e.g., hepatocellular carcinoma cells) typically express low levels of miR-122 as compared to normal liver cells. Therefore, a nucleic acid molecule (e.g., RNA, e.g., mRNA) encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the nucleic acid molecule (e.g., RNA, e.g., mRNA) includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 75, wherein the miRNA binding site binds to miR-122. In another particular aspect, the miRNA binding site comprises a nucleotide sequence at least 80%, at least 85%, at least 90%, at least 95%, or 100% identical to SEQ ID NO: 73, wherein the miRNA binding site binds to miR-122. These sequences are shown below in Table 19.

In some embodiments, a nucleic acid molecule (e.g., RNA, e.g., mRNA) of the disclosure comprises a miRNA binding site, wherein the miRNA binding site comprises one or more nucleotide sequences selected from Table 19, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 19, including any combination thereof. In some embodiments, the miRNA binding site binds to miR-142 or is complementary to miR-142. In some embodiments, the miR-142 comprises SEQ ID NO: 66. In some embodiments, the miRNA binding site binds to miR-142-3p or miR-142-5p. In some embodiments, the miR-142-3p binding site comprises SEQ ID NO: 68. In some embodiments, the miR-142-5p binding site comprises SEQ ID NO: 70. In some embodiments, the miRNA binding site comprises a nucleotide sequence at least 80%, at least 85%, at least 90%, at least 95%, or 100% identical to SEQ ID NO: 68 or SEQ ID NO: 70.

TABLE 19
Representative microRNAs and
microRNA binding sites

| SEQ ID | | |
|---|---|---|
| NO. | Description | Sequence |
| 66 | mmiR-142 | GACAGUGCAGUCACCCAUAAAGUAGAAAG |
| | | CACUACUAACAGCACUGGAGGGUGUAGUG |
| | | UUUCCUACUUUAUGGAUGAGUGUACUGUG |
| 67 | mmiR-142- | UGUAGUGUUUCCUACUUUAUGGA |
| | 3p | |
| 68 | mmiR-142- | UCCAUAAAGUAGGAAACACUACA |
| | 3p binding | |
| | site | |
| 69 | mmiR-142- | CAUAAAGUAGAAAGCACUACU |
| | 5p | |
| 70 | mmiR-142- | AGUAGUGCUUUCUACUUUAUG |
| | 5p binding | |
| | site | |
| 71 | miR-122 | CCUUAGCAGAGCUGUGGAGUGUGACAAUG |
| | | GUGUUUGUGUCUAAACUAUCAAACGCCAU |
| | | UAUCACACUAAAUAGCUACUGCUAGGC |
| 72 | miR-122-3p | AACGCCAUUAUCACACUAAAUA |
| 73 | miR-122-3p | UAUUUAGUGUGAUAAUGGCGUU |
| | binding site | |
| 74 | miR-122-5p | UGGAGUGUGACAAUGGUGUUUG |
| 75 | miR-122-5p | CAAACACCAUUGUCACACUCCA |
| | binding site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure. miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a nucleic acid molecule (e.g., RNA, e.g., mRNA) of the disclosure can be engineered to include more than one miRNA site expressed in different tissues or different cell types of a subject. As a non-limiting example, a nucleic acid molecule (e.g., RNA, e.g., mRNA) of the disclosure can be engineered to include miR-192 and miR-122 to regulate expression of the nucleic acid molecule (e.g., RNA, e.g., mRNA) in the liver and kidneys of a subject. In another embodiment, a nucleic acid molecule (e.g., RNA, e.g., mRNA) of the disclosure can be engineered to include more than one miRNA site for the same tissue. In some embodiments, the therapeutic window and or differential expression associated with the polypeptide encoded by a nucleic acid molecule (e.g., RNA, e.g., mRNA) of the disclosure can be altered with a miRNA binding site. For example, a nucleic acid molecule (e.g., RNA, e.g., mRNA) encoding a polypeptide that provides a death signal can be designed to be more highly expressed in cancer cells by virtue of the miRNA signature of those cells. Where a cancer cell expresses a lower level of a particular miRNA, the nucleic acid molecule (e.g., RNA, e.g., mRNA) encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple nucleic acid molecule (e.g., RNA, e.g., mRNA) can be designed and administered having different signals based on the use of miRNA binding sites as described herein.

In some embodiments, the expression of a nucleic acid molecule (e.g., RNA, e.g., mRNA) of the disclosure can be controlled by incorporating at least one sensor sequence in the polynucleotide and formulating the nucleic acid molecule (e.g., RNA, e.g., mRNA) for administration. As a non-limiting example, a nucleic acid molecule (e.g., RNA, e.g., mRNA) of the disclosure can be targeted to a tissue or cell by incorporating a miRNA binding site and formulating the nucleic acid molecule (e.g., RNA, e.g., mRNA) in a lipid nanoparticle comprising a cationic lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced down modulation of the nucleic acid molecule (e.g., RNA, e.g., mRNA). In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop. In another embodiment, a miRNA seed sequence can be incorporated in the loop of a stem loop and a miRNA binding site can be incorporated into the 5′ or 3′ stem of the stem loop. In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a nucleic acid molecule (e.g., RNA, e.g., mRNA) of the disclosure can comprise at least one miRNA sequence. The miRNA sequence can be, but is not limited to, a 19 or 22 nucleotide sequence and/or a miRNA sequence without the seed. In one embodiment the miRNA sequence in the 5′UTR can be used to stabilize a nucleic acid molecule (e.g., RNA, e.g., mRNA) of the disclosure described herein.

In another embodiment, a miRNA sequence in the 5′UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site. In some embodiments, a nucleic acid molecule (e.g., RNA, e.g., mRNA) of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure to dampen antigen presentation is miR-142-3p.

In some embodiments, a nucleic acid molecule (e.g., RNA, e.g., mRNA) of the disclosure can include at least one miRNA in order to dampen expression of the encoded polypeptide in a tissue or cell of interest. As a non-limiting example, a nucleic acid molecule (e.g., RNA, e.g., mRNA) of the disclosure can include at least one miR-122 binding site in order to dampen expression of an encoded polypeptide of interest in the liver. As another non-limiting example a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule (e.g., RNA, e.g., mRNA) that can interact with a RNA binding protein.

In some embodiments, the nucleic acid molecule (e.g., RNA, e.g., mRNA) of the disclosure comprising (i) a sequence-optimized nucleotide sequence (e.g., an ORF) and (ii) a miRNA binding site (e.g., a miRNA binding site that binds to miR-142).

In some embodiments, the nucleic acid molecule (e.g., RNA, e.g., mRNA) of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uricil in a uracil-modified sequence encoding a polypeptide is 5-methoxyuridine. In some embodiments, the nucleic acid molecule (e.g., RNA, e.g., mRNA) comprising a nucleotide sequence encoding a polypeptide disclosed herein and a miRNA binding site is formulated with a delivery agent, e.g., a compound having the Formula (I), e.g., any of Compounds 1-147.

Modified RNA Molecules Comprising Functional RNA Elements

The present disclosure provides synthetic nucleic acid molecules (e.g., RNA, e.g., mRNA) comprising a modification (e.g., an RNA element), wherein the modification provides a desired translational regulatory activity. In some embodiments, the disclosure provides a nucleic acid molecule (e.g., RNA, e.g., mRNA) comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43 S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a nucleic acid molecule (e.g., RNA, e.g., mRNA), comprising an RNA element that comprises a sequence and/or an RNA secondary structure(s) that provides a desired translational regulatory activity as described herein. In some aspects, the nucleic acid molecule (e.g., RNA, e.g., mRNA) comprises an RNA element that comprises a sequence and/or an RNA secondary structure(s) that promotes and/or enhances the translational fidelity of translation. In some aspects, the nucleic acid molecule (e.g., RNA, e.g., mRNA) comprises an RNA element that comprises a sequence and/or an RNA secondary structure(s) that provides a desired translational regulatory activity, such as inhibiting and/or reducing leaky scanning. In some aspects, the disclosure provides a nucleic acid molecule (e.g., RNA, e.g., mRNA) that comprises an RNA element that comprises a sequence and/or an RNA secondary structure(s) that inhibits and/or reduces leaky scanning thereby promoting the translational fidelity of the nucleic acid molecule (e.g., RNA, e.g., mRNA).

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides a nucleic acid molecule (e.g., RNA, e.g., mRNA) having one or more structural modifications that inhibits leaky scanning and/or promotes the translational fidelity of translation, wherein at least one of the structural modifications is a GC-rich RNA element. In some aspects, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA). In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA). In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the nucleic acid molecule (e.g., RNA, e.g., mRNA).

In some embodiments, the GC-rich RNA element comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In some embodiments, the GC-rich RNA element comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, a GC-rich RNA element comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In some embodiments, a GC-rich RNA element comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA), and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA), and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 177). In some embodiments, the sequence comprises a repeating GC-motif [CCG]n, wherein n=5 (SEQ ID NO: 178).

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element comprises any one of the sequences set forth in Table 20. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA). In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V1 [CCCCGGCGCC] (SEQ ID NO: 80) as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence V1 as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence V1 as set forth in Table 5 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence VI as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V2 [CCCCGGC] as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence V2 as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence V2 as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence V2 as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence EK [GCCGCC] as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence EK as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence EK as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence EK as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V1 [CCCCGGCGCC] (SEQ ID NO: 80) as set forth in Table 20, or derivatives or analogs thereof, preceding a Kozak consensus sequence in the 5′ UTR of the nucleic acid molecule (e.g., RNA, e.g., mRNA), wherein the 5′ UTR comprises the following sequence shown in Table 20:

(SEQ ID NO: 77)
GGGAAATAAGAGAGAAAAGAAGAGTAAGAAGAAATATAAGA.

In some embodiments, the GC-rich element comprises the sequence V1 as set forth in Table 20 located immediately adjacent to and upstream of the Kozak consensus sequence in the 5′ UTR sequence shown in Table 20. In some embodiments, the GC-rich element comprises the sequence VI as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA), wherein the 5′ UTR comprises the following sequence shown in Table 20:

(SEQ ID NO: 77)
GGGAAATAAGAGAGAAAAGAAGAGTAAGAAGAAATATAAGA.

In other embodiments, the GC-rich element comprises the sequence V1 as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA), wherein the 5′ UTR comprises the following sequence shown in Table 20:

(SEQ ID NO: 77)
GGGAAATAAGAGAGAAAAGAAGAGTAAGAAGAAATATAAGA.

In some embodiments, the 5′ UTR comprises the following sequence set forth in Table 20:

(SEQ ID NO: 78)
GGGAAATAAGAGAGAAAAGAAGAGTAAGAAGAAATATAAGACCCCGGC
GCCGCCACC

In some embodiments, the 5′ UTR comprises the following sequence set forth in Table 20:

(SEQ ID NO: 79)
GGGAAATAAGAGAGAAAAGAAGAGTAAGAAGAAATATAAGACCCCGGC
GCCACC

TABLE 20

| SEQ ID | | |
|---|---|---|
| NO: | 5′ UTRs | 5′ UTR Sequence |
| 76 | Standard | GGGAAATAAGAGAGAAAAGAAGAGTAAGAAGAA |
| | | ATATAAGAGCCACC |
| 77 | UTR | GGGAAATAAGAGAGAAAAGAAGAGTAAGAAGAA |
| | | ATATAAGA |
| 78 | V1-UTR | GGGAAATAAGAGAGAAAAGAAGAGTAAGAAGAA |
| | | ATATAAGACCCCGGCGCCGCCACC |
| 79 | V2-UTR | GGGAAATAAGAGAGAAAAGAAGAGTAAGAAGAA |
| | | ATATAAGACCCCGGCGCCACC |

In some embodiments, the disclosure provides a modified nucleic acid molecule (e.g., RNA, e.g., mRNA) comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In some embodiments, the stable RNA secondary structure is upstream of the Kozak consensus sequence. In some embodiments, the stable RNA secondary structure is located about 30, about 25, about 20, about 15, about 10, or about 5 nucleotides upstream of the Kozak consensus sequence. In some embodiments, the stable RNA secondary structure is located about 20, about 15, about 10 or about 5 nucleotides upstream of the Kozak consensus sequence. In some embodiments, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In some embodiments, the modification is operably linked to an open reading frame encoding a polypeptide and wherein the modification and the open reading frame are heterologous.

In some embodiments, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of nucleic acid molecule (e.g., RNA, e.g., mRNA), by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along a nucleic acid molecule (e.g., RNA, e.g., mRNA), footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

Agents for Reducing Protein Expression

In one embodiment, the agent associated with/encapsulated by the lipid-based composition (e.g., LNP) is an agent that reduces (i.e., decreases, inhibits, downregulates) protein expression. In one embodiment, the agent reduces protein expression in the target immune cell (e.g., T cell, B cell, dendritic cell, myeloid cell) to which the lipid-based composition is delivered. Additionally or alternatively, in another embodiment, the agent results in reduced protein expression in other cells, e.g., bystander cells, than the target immune cell (e.g., T cell, B cell, dendritic cell, myeloid cell) to which the lipid-based composition is delivered. Non-limiting examples of types of agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

RNA Interference Molecules

RNA interference (RNAi) refers to a biological process in which RNA molecules inhibit gene expression or translation by neutralizing targeted mRNA molecules. RNAi is a gene silencing process that is controlled by the RNA-induced silencing complex (RISC) and is initiated by short double-stranded RNA molecules (dsRNA) in a cell's cytoplasm. Two types of small ribonucleic acid molecules, small interfering RNAs (siRNAs) and microRNAs (miRNAs), are central to RNA interference. While RNAi is a natural cellular process, the components of RNAi also have been synthesized and exploited for inhibiting expression of target genes/mRNAs of interest in vitro and in vivo.

As a natural process, dsRNA initiates RNAi by activating the ribonuclease protein Dicer, which binds and cleaves dsRNA and short hairpin RNAs (shRNAs) to produce double-stranded fragments of 20-25 base pairs. These short double-stranded fragments are called small interfering RNAs (siRNAs). These siRNAs are then separated into single strands and integrated into an active RISC, by the RISC-Loading Complex (RLC). After integration into the RISC, siRNAs base-pair to their target mRNA and cleave it, thereby preventing it from being used as a translation template.

The phenomenon of RNAi, broadly defined, also includes the gene silencing effects of miRNAs. MicroRNAs are genetically-encoded non-coding RNAs that help regulate gene expression, for example during development. Naturally-occurring mature miRNAs are structurally similar to siRNAs produced from exogenous dsRNA, but before reaching maturity, miRNAs undergo extensive post-transcriptional modification, including a dsRNA portion of pre-miRNA being cleaved by Dicer to produce the mature miRNA molecule that can be integrated into the RISC complex.

Accordingly, in one embodiment, the agent associated with/encapsulated by the lipid-based composition, e.g., LNP, is an RNAi molecule (i.e., a molecule that mediates or is involved in RNA interference), including siRNAs and miRNAs, each of which is described in further detail below.

Small Interfering RNAs

Small interfering RNAs (siRNAs), also referred to as short interfering RNAs or silencing RNAs, are a class of double-stranded RNA molecules, typically 20-25 base pairs in length, that operate within the RNAi pathway to interfere with the expression of specific target sequences with complementary nucleotide sequences. siRNAs inhibit gene expression by degrading mRNA after transcription, thereby preventing translation. As used herein, the term “siRNA” encompasses all forms of siRNAs known in the art, including, but not limited to, shortmers, longmers, 2′5′-isomers and Dicer-substrate RNAs. Naturally-occurring and artificially synthesized siRNAs, and their use in therapy (e.g., delivered by nanoparticles), have been described in the art (see e.g., Hamilton and Balcombe (1999) Science 286:950-952; Elbashir et al. (2001) Nature 411:494-498; Shen et al. (2012) Cancer Gene Therap. 19:367-373; Wittrup et al. (2015) Nat. Rev. Genet. 16:543-552).

Accordingly, in one embodiment, the agent associated with/encapsulated by the lipid-based composition, e.g., LNP, is an siRNA. In one embodiment, the siRNA inhibits expression of a target sequence expressed in immune cells. In one embodiment, the siRNA inhibits expression of a target sequence expressed in T cells. In one embodiment, the siRNA inhibits expression of a target sequence expressed in B cells. In one embodiment, the siRNA inhibits expression of a target sequence expressed in dendritic cells. In one embodiment, the siRNA inhibits expression of a target sequence expressed in myeloid cells.

In another embodiment, the siRNA inhibits the expression of a transcription factor (e.g., FoxP3, T-bet, RoRgt, STAT3, AhR, NFkB) in the immune cell (e.g., T cells, B cells, dendritic cells, myeloid cells). In one embodiment, the siRNA inhibits the expression of a cytoplasmic protein (e.g., Mcl-1, HDAC10 histone deacetylase, asparaginyl endopeptidase (AEP), SOCS1, SOCS2, PPARg, GILZ, AMKa1, AMKa2, SHP-1, SHP-2, CAMKK2, IDO1, IDO2, TDO) in the immune cell (e.g., T cells, B cells, dendritic cells, myeloid cells). In another embodiment, the siRNA inhibits the expression of a transmembrane protein (e.g., cell surface receptors, such as antibodies, T cell receptors, immune checkpoint inhibitors) in the immune cell (e.g., T cells, B cells, dendritic cells, myeloid cells). In another embodiment, the siRNA inhibits the expression of a secreted protein (e.g., cytokines, chemokines) in the immune cell (e.g., T cells, B cells, dendritic cells, myeloid cells). In another embodiment, the siRNA inhibits the expression of an intracellular signaling protein in the immune cell (e.g., T cells, B cells, dendritic cells, myeloid cells). In another embodiment, the siRNA inhibits the expression of an enzyme (e.g., AMPKa1, AMPKa2, HDAC10, AEP, SHP-1, SHP-2, CAMKK2, IDO1, IDO2, TDO) in the immune cell (e.g., T cells, B cells, dendritic cells, myeloid cells).

MicroRNAs

MicroRNAs (miRNAs) are small non-coding RNA molecules (typically containing about 22 nucleotides) that function in RNA silencing and post-transcriptoinal regulation of gene expression. miRNAs inhibit gene expression via base-pairing with complementary sequences within mRNA molecules, leading to cleavage of the mRNA, destabilization of the mRNA through shortening of its polyA tail and/or less efficient translation of the mRNA into protein by ribosomes. With respect to mRNA cleavage, it has been demonstrated that given complete complementarity between the miRNA and the target mRNA sequence, the protein Ago2 can cleave the mRNA, leading to direct mRNA degradation. miRNAs and their function have been described in the art (see e.g., Ambros (2004) Nature 431:350-355; Bartel (2004) Cell 116:281-297; Bartel (2009) Cell 136:215-233; Fabian et al. (2010) Ann. Rev. Biochem. 79:351-379).

Accordingly, in one embodiment, the agent associated with/encapsulated by the lipid-based composition, e.g., LNP, is a miRNA. In one embodiment, the miRNA inhibits expression of a target sequence expressed in immune cells. In one embodiment, the miRNA inhibits expression of a target sequence expressed in T cells. In one embodiment, the miRNA inhibits expression of a target sequence expressed in B cells. In one embodiment, the miRNA inhibits expression of a target sequence expressed in dendritic cells. In one embodiment, the miRNA inhibits expression of a target sequence expressed in myeloid cells.

In another embodiment, the miRNA inhibits the expression of a transcription factor (e.g., FoxP3, T-bet, RoRgt, STAT3, AhR, NFkB) in the immune cell (e.g., T cells, B cells, dendritic cells, myeloid cells). In one embodiment, the miRNA inhibits the expression of a cytoplasmic protein (e.g., Mcl-1, HDAC10 histone deacetylase, asparaginyl endopeptidase (AEP), SOCS1, SOCS2, PPARg, GILZ, AMKa1, AMKa2, SHP-1, SHP-2, CAMKK2, IDO1, IDO2, TDO) in the immune cell (e.g., T cells, B cells, dendritic cells, myeloid cells). In another embodiment, the miRNA inhibits the expression of a transmembrane protein (e.g., cell surface receptors, such as antibodies, T cell receptors, immune checkpoint inhibitors) in the immune cell (e.g., T cells, B cells, dendritic cells, myeloid cells). In another embodiment, the miRNA inhibits the expression of a secreted protein (e.g., cytokines, chemokines) in the immune cell (e.g., T cells, B cells, dendritic cells, myeloid cells). In another embodiment, the miRNA inhibits the expression of an intracellular signaling protein in the immune cell (e.g., T cells, B cells, dendritic cells, myeloid cells). In another embodiment, the miRNA inhibits the expression of an enzyme (e.g., AMPKa1, AMPKa2, HDAC10, AEP, SHP-1, SHP-2, CAMKK2, IDO1, IDO2, TDO) in the immune cell (e.g., T cells, B cells, dendritic cells, myeloid cells).

For modulation of immune cell activity and/or modulation of immune responses, non-limiting examples of suitable miRNAs include Let-7d-5p, miR-7, miR-10a, miR-10b, miR-15, miR-18a, miR-20a, miR-20b, miR-21, miR-26a, miR-34a, miR-96, miR-99a, miR-100, miR-124, miR-125a, miR-126, miR-142-3p, miR-146, miR-150, miR-155, miR-181a and miR-210.

Antagomirs

Antagomirs, also known in the art as anti-miRs or blockmirs, are a class of chemically engineered oligonucleotides that prevent other molecules from binding to a desired site on an mRNA molecule. Antagomirs are used to silence endogenous miRNAs. An antagomir is a small synthetic RNA that is perfectly complementary to the specific miRNA target, with either mispairing at the cleavage site of Ago2 or some sort of base modification to inhibit Ago2 cleavage. Typically, antagomirs have one or more modifications, such as 2′-methoxy groups and/or phosphorothioates, to make them more resistant to degradation. Antagomirs and their function have been described in the art (see e.g., Krutzfeldt et al. (2005) Nature 438:685-689; Czech (2006) New Eng. J. Med. 354:1194-1195).

Accordingly, in one embodiment, the agent associated with/encapsulated by the lipid-based composition, e.g., LNP, is an antagomir. Since antagomirs block (inhibit) the activity of endogenous miRNAs that downregulate gene expression, the effect of an antagomir can be to enhance (i.e., increase, stimulate, upregulate) expression of a gene of interest. Accordingly, in one embodiment, the antagomir enhances expression of a target sequence expressed in immune cells. In one embodiment, the antagomir enhances expression of a target sequence expressed in T cells. In one embodiment, the antagomir enhances expression of a target sequence expressed in B cells. In one embodiment, the antagomir enhances expression of a target sequence expressed in dendritic cells. In one embodiment, the antagomir expression of a target sequence expressed in myeloid cells.

In another embodiment, the antagomir enhances the expression of a transcription factor (e.g., FoxP3, T-bet, RoRgt, STAT3, AhR, NFkB) in the immune cell (e.g., T cells, B cells, dendritic cells, myeloid cells). In one embodiment, the antagomir enhances the expression of a cytoplasmic protein (e.g., Mcl-1, HDAC10 histone deacetylase, asparaginyl endopeptidase (AEP), SOCS1, SOCS2, PPARg, GILZ, AMKa1, AMKa2, SHP-1, SHP-2, CAMKK2, IDO1, IDO2, TDO) in the immune cell (e.g., T cells, B cells, dendritic cells, myeloid cells). In another embodiment, the antagomir enhances the expression of a transmembrane protein (e.g., cell surface receptors, such as antibodies, T cell receptors, immune checkpoint inhibitors) in the immune cell (e.g., T cells, B cells, dendritic cells, myeloid cells). In another embodiment, the antagomir enhances the expression of a secreted protein (e.g., cytokines, chemokines) in the immune cell (e.g., T cells, B cells, dendritic cells, myeloid cells). In another embodiment, the antagomir enhances the expression of an intracellular signaling protein in the immune cell (e.g., T cells, B cells, dendritic cells, myeloid cells). In another embodiment, the antagomir enhances the expression of an enzyme (e.g., AMPKa1, AMPKa2, HDAC10, AEP, SHP-1, SHP-2, CAMKK2, IDO1, IDO2, TDO) in the immune cell (e.g., T cells, B cells, dendritic cells, myeloid cells).

For modulation of immune cell activity and/or modulation of immune responses, non-limiting examples of suitable antagomirs include those that specifically target miRNAs selected from miR-7, miR-15a, miR-16, miR-17, miR-21, miR-22, miR-23, miR-24, miR-25, miR-27, miR-31, miR-92, miR-106b, miR-146b, miR-148a, miR-155 and miR-210.

Antisense RNAs

Antisense RNAs (asRNAs), also referred to in the art as antisense transcripts, are naturally-occurring or synthetically produced single-stranded RNA molecules that are complementary to a protein-coding messenger RNA (mRNA) with which it hybridizes and thereby blocks the translation of the mRNA into a protein. Antisense transcript are classified into short (less than 200 nucleotides) and long (greater than 200 nucleotides) non-coding RNAs (ncRNAs). The primary natural function of asRNAs is in regulating gene expression and synthetic versions have been used widely as research tools for gene knockdown and for therapeutic applications. Antisense RNAs and their functions have been described in the art (see e.g., Weiss et al. (1999) Cell. Molec. Life Sci. 55:334-358; Wahlstedt (2013) Nat. Rev. Drug Disc. 12:433-446; Pelechano and Steinmetz (2013) Nat. Rev. Genet. 14:880-893). Accordingly, in one embodiment, the agent associated with/encapsulated by the lipid-based composition, e.g., LNP, is a nucleic acid (e.g., RNA or DNA) that encodes or that is an antisense RNA.

Ribozymes

Ribozymes (ribonucleic acid enzymes) are RNA molecules that are capable of catalyzing biochemical reactions, similar to the action of protein enzymes. The most common activities of natural or in vitro-evolved ribozymes are the cleavage or ligation of RNA and DNA and peptide bond formation. Moreover, self-cleaving RNAs that have good enzymatic activity have been described in the art. Therapeutic use of ribozymes, in particular for the cleavage of RNA-based viruses, is under development. Ribozymes and their functions have been described in the art (see e.g., Kruger et al. (1982) Cell 31:147-157; Tang and Baker (2000) Proc. Natl. Acad. Sci. USA 97:84-89; Fedor and Williamson (2005) Nat. Rev. Mol. Cell. Biol. 6:399-412). Accordingly, in one embodiment, the agent associated with/encapsulated by the lipid-based composition, e.g., LNP, is a nucleic acid (e.g., RNA or DNA) that encodes or that is a ribozyme.

Small Hairpin RNAs

Small (or short) hairpin RNA (shRNA) is a type of synthetic RNA molecule with a tight hairpin turn that can be used to silence target gene expression via RNA interference. shRNA is an advantageous mediator of RNA interference in that it has a relatively low rate of degradation and turnover. Expression of shRNA in cells typically is accomplished by delivery of plasmids or through viral vectors (e.g., adeno-associated virus, adenovirus or lentivirus vectors) or bacterial vectors encoding the shRNA. shRNAs and their use in gene therapy has been described in the art (see e.g., Paddison et al. (2002) Genes Dev. 16:948-958; Xiang et al. (2006) Nat. Biotech. 24:697-702; Burnett et al. (2012) Biotech. Journal 6:1130-1146). Accordingly, in one embodiment, the agent associated with/encapsulated by the lipid-based composition, e.g., LNP, is a nucleic acid (e.g., RNA or DNA) that encodes or that is an shRNA.

Locked Nucleic Acids

Locked nucleic acids, also referred to as inaccessible RNA, are modified RNA nucleotide molecules in which the ribose moiety of the LNA is modified with an extra bridge connecting the 2′ oxygen and the 4′ carbon. This bridge “locks” the ribose in the 3′-endo (North) conformation. LNA nucleotides can be mixed with DNA or RNA residues in an oligonucleotide whenever desired and hybridize with DNA or RNA according to Watson-Crick base-pairing rules. The locked ribose conformation enhances base stacking and backbone pre-organization. This significantly increases the hybridization properties (e.g., melting temperature) of oligonucleotides containing LNA nucleotides. LNA molecules, and their properties, have been described in the art (see e.g., Obika et al. (1997) Tetrahedron Lett. 38:8735-8738; Koshkin et al. (1998) Tetrahedron 54:3607-3630; Elmen et al. (2005) Nucl. Acids Res. 33:439-447). Accordingly, in one embodiment, the agent associated with/encapsulated by the lipid-based composition, e.g., LNP, is a nucleic acid (e.g., RNA or DNA) comprising one or more locked nucleic acid (LNA) nucleotides.

CRISPR/Cas9 Agents

In some embodiments, the lipid-based compositions (e.g., lipid nanoparticle) described herein are useful in methods involving the CRISPR (Clustered Regularly Interspaced Short Palindromic Repeats)-Cas9 system. CRISPR/Cas9 is used to edit the genome, wherein the enzyme Cas9 makes cuts in the DNA and allows new genetic sequences to be inserted. Single-guide RNAs are used to direct Cas9 to the specific spot in DNA where cuts are desired. However, it has been reported that genome editing in immune cells, in particular T cells, is a significant challenge.

Recent studies have shown the ability of CRISPR/Cas9 to downregulate CXCR4 and PD-1 expression of T cells by electroporating the cells in vitro (Schumann, K. et al. PNAS, Vol. 112(33): 10437-10443, Aug. 18, 2015; Rupp, L. et al., Scientific Reports, Vol. 7, Article number 737, 2017; each of which is herein incorporated by reference). There remains a need to introduce the CRISPR/Cas9 into immune cells (e.g., T cells, B cells, dendritic cells, myeloid cells) in vivo. Accordingly, the present disclosure provides methods of editing the genome of immune cells (e.g., T cells, B cells, dendritic cells, myeloid cells) with the CRISPR/Cas9 system by using the lipid-based compositions comprising immune delivery lipids, as described herein. Accordingly, in some embodiments, the agent(s) that is associated with/encapsulated by the lipids (e.g., LNP) is one or more components of the CRISPR/Cas9 system. For example, the Cas9 enzyme and single-guide RNA can be associated with/encapsulated in the lipid-based compositions described herein. Optionally, genetic material of interest to be modified (e.g., DNA) can also be encapsulated in the lipid-based composition or, alternatively, the CRISPR/Cas9 system delivered by the lipid-based composition can act on endogenous genetic material of interest in the target immune cells (e.g., T cells, B cells, dendritic cells, myeloid cells).

### Exemplary Target Proteins

The molecule targeted (e.g., encoded by the nucleic acid in the LNP or targeted for knock down) can be chosen based on the desired outcome. Given that the LNPs of the invention have now been found to target immune cells, one of ordinary skill in the art can deliver numerous art recognized immunomodulatory molecules to immune cells to enhance or reduce immune responses. Exemplary such immunomodulatory molecules (e.g., nucleic acid molecules such as DNA, RNA, mRNA, RNAi) are well known in the art and exemplary targets for such molecules are also well known in the art and exemplary such molecules are disclosed herein. When expressing proteins (e.g., using mRNA), such proteins can be a full-length protein or, alternatively, a functional fragment thereof (e.g., a fragment of the full-length protein that includes one or more functional domains such that the functional activity of the full-length protein is retained). Furthermore, in certain embodiments, the protein encoded by a nucleic acid in the LNP can be a modified protein, e.g., can comprise one or more heterologous domains, e.g., the protein can be a fusion protein that contains one more domains that do not naturally occur in the protein such that the function of the protein is altered. An example of a protein comprising a heterologous domain is a chimeric antigen receptor (described further below).

Specific proteins (e.g., cytokines, chemokines, costimulatory molecules, TcRs, CARs, recruitment factors, transcription factors, effector molecules) that can be encoded by the nucleic acid (e.g., mRNA) or inhibited by a nucleic acid molecule (e.g., siRNA, miRNAs) to thereby modulate immune responses (upregulation or downregulation) are described in detail in the following subsection.

Induction or reduction of a protein of interest in or on an immune cell can be measured by standard methods known in the art, such as by immunofluorescence or flow cytometry.

### Naturally Occurring Targets

In one embodiment, the agent associated with/encapsulated by the lipid-based composition, e.g., LNP, modulates a naturally-occurring target (e.g., up- or down-regulates the activity of a naturally-occurring target) of an immune cell (e.g., a T cell, B cell, myeloid cell, dendritic cell). The agent may itself encode the naturally-occurring target, or may function to modulate a naturally-occurring target (e.g., in a cell in vivo, such as in a subject). The naturally-occurring target can be a full-length target, such as a full-length protein, or can be a fragment or portion of a naturally-occurring target, such as a fragment or portion of a protein. The agent that modulates a naturally-occurring target (e.g., by encoding the target itself or by functioning to modulate the activity of the target) can act in an autocrine fashion, i.e., the agent exerts an effect directly on the cell into which the agent is delivered. Additionally or alternatively, the agent that modulates a naturally-occurring target can function in a paracrine fashion, i.e., the agent exerts an effect indirectly on a cell other than the cell into which the agent is delivered (e.g., delivery of the agent into one type of cell results in secretion of a molecule that exerts effects on another type of cell, such as bystander cells). Agents that modulate naturally-occurring targets include nucleic acid molecules that induce (e.g., enhance, stimulate, upregulate) protein expression, such as mRNAs and DNA. Agents that modulate naturally-occurring targets also include nucleic acid molecules that reduce (e.g., inhibit, decrease, downregulate) protein expression, such as siRNAs, miRNAs and antagomirs. Non-limiting examples of naturally-occurring targets include soluble proteins (e.g., secreted proteins), intracellular proteins (e.g., intracellular signaling proteins, transcription factors) and membrane-bound or transmembrane proteins (e.g., receptors).

### Soluble Targets

In one embodiment, the agent associated with/encapsulated by the lipid-based composition, e.g., LNP, modulates the activity of a naturally-occurring soluble target, for example by encoding the soluble target itself or by modulating the expression (e.g., transcription or translation) of the soluble target in an immune cell (e.g., T cell, B cell, dendritic cell, myeloid cell). In one embodiment, the cell is a lymphocyte. Non-limiting examples of naturally-occurring soluble targets include cytokines and chemokines. Suitable cytokines and chemokines for particular uses in stimulating or inhibiting immune responses are described further below. As demonstrated in Example 19, the lipid-based compositions of the disclosure are effective at delivering mRNA encoding a soluble target into immune cells (e.g., T cells) such that the soluble target is expressed by the immune cells.

In one embodiment, the method of using the lipid-based composition, e.g. LNP, is used to stimulate (upregulate, enhance) the activation or activity of an immune cell, for example in situations where stimulation of an immune response is desirable, such as in cancer therapy or treatment of an infectious disease (e.g., a viral, bacterial, fungal, protozoal or parasitic infection). In another embodiment, the method of using the lipid-based composition, e.g. LNP, is used to inhibit (downregulate, reduce) the activation or activity of an immune cell, for example in situations where inhibition of an immune response is desirable, such as in autoimmune diseases, allergies and transplantion.

In one embodiment of stimulating the activation or activity of an immune cell, the target protein is a cytokine. Cytokines are mediators of intracellular signaling that regulate the immune system Non-limiting examples of cytokines that can stimulate immune cell activation or activity include IL-1 (pro-inflammatory cytokine), IL-2 (T cell growth factor that promotes T cell differentiation), IL-3 (stimulates proliferentiation of myeloid lineage cells), IL-4 (stimulates B and T cell proliferation and B cell differentiation), IL-5 (stimulates B cell growth), IL-6 (pro-inflammatory), IL-7 (stimulates differentiation of lymphoid lineage cells), IL-12 (differentiation of naïve T cells to Th1 cells), IL-13 (stimulation of activated B and T cell proliferation and B cell differentiation), IL-15 (regulation of activation and proliferation of T cells and NK cells), IL-17 (proinflammatory and induces chemokines), IL-18 (proinflammatory and promotes IFN release), IL-21 (proinflammatory and regulates NK and CTL proliferation), IL-23 (proinflammatory), TNFα (stimulates systemic inflammation and inhibits tumorigenesis and viral replication), TNFβ (regulates development of secondary lymphoid organs), IFNα involved in innate immunity to viral infection) IFNβ (involved in innate immunity to viral infection) IFNγ (involved in innate and adaptive immunity to viruses and other infectious agents), GM-CSF (stimulates white blood cell production and enhances anti-tumor T cells), G-CSF (stimulates white blood cell production), and a combination thereof.

In one embodiment, the cytokine is a pro-inflammatory cytokine, non-limiting examples of which include IL-1, IL-6, IL-17, IL-18, IL-23, TNFα, IFN-α, IFN-β and IFN-γ. A pro-inflammatory cytokine can be used in situations in which stimulation of an inflammatory response is desired, for example to increase anti-tumor immunity in cancer therapy or in viral infections. In one embodiment, the cytokine promotes T cell activation. Non-limiting examples of cytokines that promote T cell activation or differentiation include IL-2, IL-4, IL-12, IL-13, IL-15, and IFN-α. In one embodiment, the cytokine promotes Th2 responses. Non-limiting examples of cytokines that promote Th2 responses include IL-4 and IL-10. In one embodiment, the cytokine promotes B cell activation. Non-limiting examples of cytokines that promote B cell activation include IL-4, IL-5, IL-6, IL-10, IL-13 and IFN.

In one embodiment of stimulating the activation or activity of an immune cell, the protein is a chemokine or a chemokine receptor. Chemokines have been demonstrated to be substances that control the trafficking of inflammatory cells (including granulocytes and monocytes/monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of Inflammation, Article ED 170381, pg. 1-15). In one embodiment, the chemokine or chemokine receptor acts on regulatory T cells, non-limiting examples of which include CCL22, CCL28, CCR4 and CCR10. In another embodiment, the chemokine or chemokine receptor acts on cytotoxic T cells, non-limiting examples of which include CXCL9, CXCL10, CXCL11 and CXCR3. In another embodiment, the chemokine or chemokine receptor acts on natural killer cells, non-limiting examples of which include CXCL9, CXCL10, CXCL11, CCL3, CCL4, CCL5, CCL2, CCL8, CCL12, CCL13, CCL19, CCL21, CX3CL1, CXCR3, CCR1, CCR5, CCR2 and CX3CR1. In another embodiment, the chemokine or chemokine receptor acts on immature dendritic cells, non-limiting examples of which include CCL3, CCL4, CCL5, CCL2, CCL7, CCL8, CCL22, CCL1, CCL17, CXCL12, CCR1, CCR2, CCR4, CCR5, CCR6, CCR8 and CXCR4. In another embodiment, the chemokine or chemokine receptor acts on mature dendritic cells, non-limiting examples of which include CCL19, CCL21, CXCL12, CCR7 and CXCR4. In another embodiment, the chemokine or chemokine receptor acts on tumor-associated macrophages, non-limiting examples of which include CCL2, CCL7, CCL8, CCL3, CCL4, CCL5, CXCL12, CCR2, CCR5 and CXCR4.

In one embodiment of stimulating the activation or activity of an immune cell, the protein is a recruitment factor. As used herein a “recruitment factor” refers to any protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8). Non-limiting examples of recruitment factors include CXCR3, CXCR5, CCR5, CCL5, CXCL10, CXCL12, CXCL16 and IFN-γ.

In one embodiment for inhibiting immune responses, the protein is an inhibitory cytokine or an antagonist of a stimulatory cytokine. In one embodiment, the inhibitory cytokine is an anti-inflammatory protein, such as IL-10 (e.g., for inflammatory bowel disease, rheumatoid arthritis and other autoimmune disease treatment), IL-11 (e.g., for inflammatory bowel disease and other autoimmune disease treatment), IFN-β (e.g., for multiple sclerosis and other autoimmune disease treatment). In another embodiment, the protein is an antagonist of any of the stimulatory cytokines listed above, such as an anti-cytokine antibody. For example, an antagonist (e.g., anti-cytokine antibody) of any of the following cytokines can be used to down modulate immune cell activity in autoimmune diseases and/or allergies: IL-5 (e.g., for allergy treatment), IL-6 (e.g., for rheumatoid arthritis and other autoimmune disease treatment), IL-12 (for autoimmunity treatment), IL-13 (e.g., for allergy treatment), IL-17 (e.g., for plaque psoriasis and other autoimmune disease treatment), IL-18 (e.g., for autoimmunity), IL-23 (e.g., for autoimmunity), TNF-α (e.g., for rheumatoid arthritis, psoriasis, inflammatory bowel disease and other autoimmune disease treatment) and IFN-γ (e.g., for autoimmunity).

Intracellular Targets

In one embodiment, the agent associated with/encapsulated by the lipid-based composition, e.g., LNP, modulates the activity of a naturally-occurring intracellular target, for example by encoding the intracellular target itself or by modulating the expression (e.g., transcription or translation) of the intracellular target in an immune cell (e.g., T cell, B cell, dendritic cell, myeloid cell). In one embodiment, the cell is a lymphoid cell. Non-limiting examples of naturally-occurring intracellular targets include transcription factors and cell signaling cascade molecules, including enzymes. Suitable transcription factors and intracellular signaling cascade molecules for particular uses in stimulating or inhibiting immune responses are described further below. As demonstrated in Example 20, the lipid-based compositions of the disclosure are effective at delivering mRNA encoding an intracellular target (e.g., a transcription factor) into immune cells (e.g., T cells) such that the intracellular target is expressed by the immune cells.

In one embodiment of stimulating the activation or activity of an immune cell, the protein target is a transcription factor. As used herein, a “transcription factor” refers to a DNA-binding protein that regulates the transcription of a gene. In one embodiment, the protein is a transcription factor that increases or polarizes an immune response. In one embodiment, the protein is a transcription factor that stimulates a Type I IFN response. In another embodiment, the protein is a transcription factor that stimulates an NFκB-mediated proinflammatory response. Non-limiting examples of transcription factors include Interferon Regulatory Factors (IRFs, including IRF-1, IRF-3, IRF-5, IRF-7, IRF-8 and IRF-9), CREB, RORg, RORgt, SOCS, NFκB, FoxP3, T-bet, STAT3 and AhR.

In one embodiment of stimulating the activation or activity of an immune cell, the protein target is an intracellular adaptor protein. In one embodiment, the intracellular adaptor protein stimulates a Type I IFN response. In another embodiment, the intracellular adaptor protein stimulates an NFκB-mediated proinflammatory response. Non-limiting examples of intacellular adaptor proteins that stimulate a Type I IFN response and/or stimulate and NFκB-mediated proinflammatory response include STING, MAVS and MyD88.

In one embodiment of stimulating the activation or activity of an immune cell, the protein target is an intracellular signaling protein. In one embodiment, the protein is an intracellular signaling protein of a TLR signaling pathway. In one embodiment, the intracellular signalling protein stimulates a Type I IFN response. In another embodiment, the intracellular signalling protein stimulates an NFκB-mediated proinflammatory response. Non-limiting examples of intracellular signalling proteins that stimulate a Type I IFN response and/or stimulate an NFκB-mediated proinflammatory response include MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK 1, and TBK 1.

Other non-limiting examples of intracellular signaling molecules for up- or downregulation of immune responses include Mcl-1, AMPKa1, AMPKa2, GILZ, PPARg, HDAC10, AEP, SHP-1, SHP-2, CAMKK2 IDO1, IDO2 and TDO.

In one embodiment of inhibiting the activation or activity of an immune cell, the protein is a transcription factor, e.g., a tolerogenic transcription factor that promotes tolerance, such as RelA, Runx1, Runx3 and FoxP3.

Membrane Bound/Transmembrane Targets

In one embodiment, the agent associated with/encapsulated by the lipid-based composition, e.g., LNP, modulates the activity of a naturally-occurring membrane-bound/transmembrane target, for example by encoding the membrane-bound/transmembrane target itself or by modulating the expression (e.g., transcription or translation) of the membrane-bound/transmembrane target in an immune cell (e.g., T cell, B cell, dendritic cell, myeloid cell). Non-limiting examples of naturally-occurring membrane-bound/transmembrane targets include costimulatory molecules, immune checkpoint molecules, homing signals and HLA molecules. Suitable membrane-bound/transmembrane targets for particular uses in stimulating or inhibiting immune responses are described further below. As demonstrated in the examples (e.g., Example 3), the lipid-based compositions of the disclosure are effective at delivering mRNA encoding a transmembrane target (e.g., a receptor ligand) into immune cells (e.g., T cells) such that the transmembrane target is expressed by the immune cells.

In one embodiment of stimulating the activation or activity of an immune cell, the protein target is a costimulatory factor that upregulates immune responses or is an antagonist of a costimulatory factor that downregulates immune responses. Non-limiting examples of costimulatory factors that upregulate immune responses include CD28, CD80, CD86, ICOS, ICOSL, OX40, OX40L, CD40, CD40L, GITR, GITRL, CD137 and CD137L. Non-limiting examples of costimulatory molecules that downregulate immune response, and thus for which an antagonist (e.g., specific antibody) can be used to thereby stimulate an immune response include PD-1, PD-L1, PD-L2 and CTLA-4. In some embodiments, the LNPs and methods of the disclosure are useful to modify effector cells (e.g., T cells) to express an mRNA encoding a universal immune receptor or UnivIR, for example, a biotin-binding immune receptor (BBIR) (see e.g., US Patent Publication US20140234348 A1 incorporated herein by reference in its entirety). Other compositions relating to universal chimeric receptors and/or effector cells expressing universal chimeric receptors are described in International Patent Applications WO2016123122A1, WO2017143094A1, WO2013074916A1, US Patent Application US20160348073A1, all of which are incorporated herein by reference in their entirety.

In one embodiment of inhibiting the activation or activity of an immune cell, the protein target is an immune checkpoint protein that down-regulates immune cells (e.g., T cells), non-limiting examples of which include CTLA-4, PD-1 and PD-L1. In another embodiment, the protein is an antagonist of a costimulatory molecule that upregulates immune responses (e.g., an antagonist antibody that binds the costimulatory molecule), non-limiting examples of which include antagonists of the following costimulatory molecules: CD28, CD80, CD86, ICOS, ICOSL, OX40, OX40L, CD40, CD40L, GITR, GITRL, CD137 and CD137L

In one embodiment, the membrane-bound/transmembrane protein target is a homing signal.

In one embodiment, the membrane-bound/transmembrane protein target is an HLA molecule, such as an HLA-G. The non-classical HLA class I molecule HLA-G is a potent inhibitory molecule that protects the cells that express it from cytolysis. This function has been reported as being crucial for the protection of the fetal cytotrophoblasts from destruction by the maternal immune system, for the protection of allografts against cytolysis by the recipients immune system and for the protection of tumors against anti-tumor immunity. Accordingly, agents that upregulate HLA-G, such as mRNA encoding HLA-G, can be used to protect cells from immune-mediated cytolysis, for example in transplant recipients. Alternatively, agents that downregulate HLA-G, such as siRNAs, miRNAs and antagomirs, can be used to promote immune-mediated cytolysis, such as in tumor-bearing subjects to stimulate anti-tumor immunity.

Modified Targets

In one embodiment, the agent associated with/encapsulated by the lipid-based composition, e.g., LNP, modulates a modified target (e.g., up- or down-regulates the activity of a non-naturally-occurring target) of an immune cell (e.g., a T cell, B cell, myeloid cell, dendritic cell). Typically, the agent itself either is or encodes the modified target. Alternatively, if a cell expresses a modified target the agent can function to modulate the activity of this modified target in the cell. The non-naturally-occurring target can be a full-length target, such as a full-length modified protein, or can be a fragment or portion of a non-naturally-occurring target, such as a fragment or portion of a modified protein. The agent that modulates a modified target can act in an autocrine fashion, i.e., the agent exerts an effect directly on the cell into which the agent is delivered. Additionally or alternatively, the agent that modulates a modified target can function in a paracrine fashion, i.e., the agent exerts an effect indirectly on a cell other than the cell into which the agent is delivered (e.g., delivery of the agent into one type of cell results in secretion of a molecule that exerts effects on another type of cell, such as bystander cells). Agents that are themselves modified targets include nucleic acid molecules, such as mRNAs or DNA, that encode modified proteins. Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

Modified Soluble Targets

In one embodiment, the agent associated with/encapsulated by the lipid-based composition, e.g., LNP, modulates a modified soluble target (e.g., up- or down-regulates the activity of a non-naturally-occurring soluble target) of an immune cell (e.g., a T cell, B cell, myeloid cell, dendritic cell). In one embodiment, the agent (e.g., mRNA) encodes a modified soluble target. In one embodiment, the modified soluble target is a soluble protein that has been modified to alter (e.g., increase or decrease) the half-life (e.g., serum half-life) of the protein. Modified soluble proteins with altered half-lifes include modified cytokines and chemokines. In another embodiment, the modified soluble target is a soluble protein that has been modified to incorporate a tether such that the soluble protein becomes tethered to a cell surface. Modified soluble proteins incorporating a tether include tethered cytokines and chemokines.

Modified Intracellular Targets

In one embodiment, the agent associated with/encapsulated by the lipid-based composition, e.g., LNP, modulates a modified intracellular target (e.g., up- or down-regulates the activity of a non-naturally-occurring intracellular target) of an immune cell (e.g., a T cell, B cell, myeloid cell, dendritic cell). In one embodiment, the cell is a lymphoid cell. In one embodiment, the agent (e.g., mRNA) encodes a modified intracellular target. In one embodiment, the modified intracellular target is a constitutively active mutant of an intracellular protein, such as a constitutively active transcription factor or intracellular signaling molecule. In another embodiment, the modified intracellular target is a dominant negative mutant of an intracellular protein, such as a dominant negative mutant of a transcription factor or intracellular signaling molecule. In another embodiment, the modified intracellular target is an altered (e.g., mutated) enzyme, such as a mutant enzyme with increased or decreased activity within an intracellular signaling cascade.

Modified Membrane Bound/Transmembrane Targets

In one embodiment, the agent associated with/encapsulated by the lipid-based composition, e.g., LNP, modulates a modified membrane-bound/transmembrane target (e.g., up- or down-regulates the activity of a non-naturally-occurring membrane-bound/transmembrane target) of an immune cell (e.g., a T cell, B cell, myeloid cell, dendritic cell). In one embodiment, the agent (e.g., mRNA) encodes a modified membrane-bound/transmembrane target. In one embodiment, the modified membrane-bound/transmembrane target is a constitutively active mutant of a membrane-bound/transmembrane protein, such as a constitutively active cell surface receptor (i.e., activates intracellular signaling through the receptor without the need for ligand binding). In another embodiment, the modified membrane-bound/transmembrane target is a dominant negative mutant of a membrane-bound/transmembrane protein, such as a dominant negative mutant of a cell surface receptor. In another embodiment, the modified membrane-bound/transmembrane target is a molecular that inverts signaling of an immune synapse (e.g., agonizes or antagonizes signaling of a T cell receptor, B cell receptor or other immune cell receptor). In another embodiment, the modified membrane-bound/transmembrane target is a chimeric membrane-bound/transmembrane protein, such as a chimeric cell surface receptor.

In one embodiment, the modified membrane-bound/transmembrane target is a chimeric antigen receptor (CAR), such as a chimeric T cell receptor, described in further detail below.

In one embodiment of stimulating the activation or activity of an immune cell, the protein target is a T cell receptor (TcR) or chimeric antigen receptor (CAR) (also known in the art as chimeric immunoreceptors, chimeric T cell receptors, artificial T cell receptors or CAR-T). In one embodiment, the protein target is a TcR or CAR that recognizes a cancer antigen. TcRs and CARs that recognize cancer antigens, and their use in cancer immunotherapy, has been described in the art (for reviews, see e.g., Newick, K. et al. (2017) Ann. Rev. Med. 68:139-152; Jackson, H. J. et al. (2016) Nat. Rev. Clin. Oncol. 13:370-383; Smith, A. J. et al. (2016) J. Cell Immunol. 2:59-68). Non-limiting examples of TcRs or CARs that recognize cancer antigens include those that bind to any of the following antigens: CD19, CD20, CD22, CD30, CD33, CD123, CD138, CD171, CEA, EGFR, HER2, mesothelin and PSMA.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain. The “chimeric antigen receptor (CAR)” is sometimes called a “chimeric receptor”, a “T-body”, or a “chimeric immune receptor (CIR).”

The phrase “CAR ligand” used interchangeably with “CAR antigen” means any natural or synthetic molecule (e.g. small molecule, protein, peptide, lipid, carbohydrate, nucleic acid) or part or fragment thereof that can specifically bind to the CAR. The “intracellular signaling domain” means any oligopeptide or polypeptide domain known to function to transmit a signal causing activation or inhibition of a biological process in a cell, for example, activation of an immune cell such as a T cell or a NK cell. Examples include ILR chain, CD28 and/or CD3ζ.

Chimeric antigen receptors (CARs) are genetically-engineered, artificial transmembrane receptors, which confer an arbitrary specificity for a ligand onto an immune effector cell (e.g., a T cell, natural killer cell or other immune cell) and which results in activation of the effector cell upon recognition and binding to the ligand. Typically these receptors are used to impart the antigen specificity of a monoclonal antibody onto an immune (e.g., T cell) to thereby target the immune cell to the desired target population.

Typically CARs contain three domains: 1) an ectodomain typically comprising a signal peptide, a ligand or antigen recognition region (e.g., scFv), and a flexible spacer; 2) a transmembrane (TM) domain; 3) an endodomain (alternatively known as an “activation domain”) typically comprising one or more intracellular signaling domains. The ectodomain of the CAR resides outside of the cell and exposed to the extracellular space, whereby it is accessible for interaction with its cognate ligand. The TM domain allows the CAR to be anchored into the cell membrane of the effector cell. The third endodomain (also known as the “activation domain”) aids in effector cell activation upon binding of the CAR to its specific ligand. Depending on effector cell type, effector cell activation can include induction of cytokine and chemokine production, as well as activation of the cytolytic activity of the cells.

Accordingly, the LNPs and methods of the disclosure provide means to enhance immune recognition and elimination of cancer cells. In one aspect the LNPs of the disclosure are useful to genetically engineer immune effector cells to express an mRNA encoding a CAR that redirects cytotoxicity toward tumor cells. CARs comprise a ligand- or antigen-specific recognition domain that binds to a specific target ligand or antigen (also referred to as a binding domain). The binding domain is typically a single-chain antibody variable fragment (scFv), a tethered ligand or the extracellular domain of a co-receptor, fused to a transmembrane domain, which is linked, in turn, to a signaling domain, typically the signaling domain derived from CD3ζ or FcRy and optionally one or more co-stimulatory domains derived from a protein such as CD28, CD137 (also known as 4-1BB), CD134 (also known as OX40) and CD278 (also known as ICOS). Engagement of the antigen binding domain of the CAR with its target antigen on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors. Typically, CARs contain a binding domain, a hinge, a transmembrane and a signaling domain derived from CD3ζ or FcRy together with one or more co-stimulatory signaling domains (e.g., intracellular co-stimulatory domains derived from CD28, CD137, CD134 and CD278). CARs have been shown to more effectively direct antitumor activity as well as increased cytokine secretion, lytic activity, survival and proliferation in CAR expressing T cells in vitro, in animal models and cancer patients (see e.g., Milone et al., Molecular Therapy, 2009; 17: 1453-1464; Zhong et al., Molecular Therapy, 2010; 18: 413-420; Carpenito et al., PNAS, 2009; 106:3360-3365; and Chang and Chen (2017) Trends Mol Med 23(5):430-450).

In some aspects, the LNPs of the disclosure expressing mRNAs encoding CARs are used to modify T cells derived from a patient with a disease or condition either ex vivo or in vivo. In one aspect, the T cells are modified to express an mRNA encoding a protein that modulates at least one effector function (e.g., a cytokine or induction of cytokines). The T cells may be cytotoxic T cells or helper T cells.

Prolonged exposure of T cells to their cognate antigen can result in exhaustion of effector functions, enabling the persistence of infected or transformed cells. Emerging evidence suggests that T cell exhaustion may also represent a significant impediment in sustaining long-lived antitumor activity by CAR T cells. Furthermore, the differentiation status of the patient-harvested T cells prior to CAR transduction and the conditioning regimen a patient undergoes before reintroducing the CAR T cells (e.g., addition or exclusion of alkylating agents, fludarabine, total-body irradiation) can also profoundly affect the persistence and cytotoxic potential of CAR T cells. In vitro culture conditions that stimulate (via anti-CD3/CD28 or stimulator cells) and expand (via cytokines, such as IL-2) T cell populations can also alter the differentiation status and effector function of CAR T cells (Ghoneim et al., (2016) Trends in Molecular Medicine 22(12): 1000-1011).

Accordingly, the LNPs and methods of the present disclosure provide new approaches toward the generation of therapeutic CAR T cells. Existing methods of therapeutic CAR-T cell preparation often requires extensive cell culture in vitro to obtain sufficient number of modified cells for adoptive cell transfer, during which natural identity or differentiation state of the T cells may have changed and T cell function may have been compromised. Furthermore, when patients are in urgent need of therapy to prevent disease progression, the time required to generate sufficient quantities of CAR T cells may not be aligned with opportunity to treat the patient, resulting in therapeutic failure and demise of patient. The LNPs and methods of the disclosure bypass these hurdles by providing an in vivo method of delivery of mRNA encoding CARs to T cells. In addition, current CAR-T cell therapy regime requires lymphocyte depletion beforehand, this weakens patient health and destroys the nourishing environment that can improve CAR-T efficacy. In some aspects, the LNPs and methods of the disclosure are useful to stimulate adoptively transferred CAR-T cells such that they can still engraft, actively proliferate and expand in vivo. For example, in one embodiment, a nucleic acid molecule that promotes lymphocyte proliferation and/or survival (e.g., a cytokine) can be delivered to immune cells to promote such expansion.

In certain embodiments, the disclosure provides CAR-T for the treatment of hematological cancers, including but not limited to acute myeloid leukemia (AML), acute lymphoblastic leukemia (ALL, e.g., B-ALL), Non-Hodgkins lymphoma (NHL) and multiple myeloma. In some embodiments, the structure of the CAR encoded by the mRNA associated with/encapsulated by the LNP comprises, from N-terminus to C-terminus, a scFv domain, a hinge & transmembrane domain (H/TM), a costimulatory (CS) domain and a TCR signaling domain.

In certain embodiments, in particular for the treatment of AML, suitable CARs target CD33 or CD123. Non-limiting examples include CARs comprising a structure: (i) an anti-CD33 scFv, a CD8 H/TM domain, an 4-1BB CS domain and a CD3C TCR signaling domain; (ii) an anti-CD33 scFv, a CD28 H/TM domain, a CD28 CS domain and a CD3C TCR signaling domain; and (iii) an anti-CD123 scFv, a CD8 H/TM domain, an 4-1BB CS domain and a CD3C TCR signaling domain. A CAR-T cell of the disclosure can be used in combination with other preparations described in the art that target CD33 or CD123, including but not limited to gemtuzumab ozogamicin, lintuzumab, vadastuximab talirine, and 2H12 (Seattle Genetics).

In certain embodiments, in particular for the treatment of ALL and/or NHL, suitable CARs target CD19 or CD20. Non-limiting examples include CARs comprising a structure: (i) an anti-CD19 scFv, a CD8 H/TM domain, an 4-1BB CS domain and a CD3C TCR signaling domain; (ii) an anti-CD19 scFv, a CD28 H/TM domain, a CD28 CS domain and a CD3C TCR signaling domain; and (iii) an anti-CD20 scFv, an IgG H/TM domain, a CD28/4-1BB CS domain and a CD3C TCR signaling domain. A CAR-T cell of the disclosure can be used in combination with other preparations described in the art that target CD19 or CD20, including but not limited to Kymriah™ (tisagenlecleucel; Novartis; formerly CTL019), Yescarta™ (axicabtagene ciloleucel; Kite Pharma) and rituximab.

In certain embodiments, in particular for the treatment of multiple myeloma, suitable CARs target BCMA. Non-limiting examples include CARs comprising a structure an anti-BCMA scFv, a CD8 H/TM domain, an 4-1BB CS domain and a CD3C TCR signaling domain. A CAR-T cell of the disclosure can be used in combination with other preparations described in the art that target BCMA, including but not limited to the BCMA-targeted CAR-T preparations described in Smith et al. (2017) American Society of Hematology Abstract No. 742 (referred to as MCARH171) and in Harrington et al. (2017) American Society of Hematology Abstract No. 1813 (referred to as JCARH125).

In some embodiments, the LNPs and methods of the disclosure are useful to modify effector cells (e.g., T cells) to express an mRNA encoding a universal, modular, anti-tag chimeric antigen receptor (UniCAR) which allows for retargeting of UniCAR engrafted immune cells against multiple antigens (see e.g., US Patent Publication US20170240612 A1 incorporated herein by reference in its entirety; Cartellieri et al., (2016) Blood Cancer Journal 6, e458 incorporated herein by reference in its entirety).

In some embodiments, the LNPs and methods of the disclosure are useful to modify effector cells (e.g., T cells) to express an mRNA encoding a switchable chimeric antigen receptor and chimeric antigen receptor effector cell (CAR-EC) switches. In this system, the CAR-EC switches have a first region that is bound by a chimeric antigen receptor on the CAR-EC and a second region that binds a cell surface molecule on target cell, thereby stimulating an immune response from the CAR-EC that is cytotoxic to the bound target cell. In some embodiments, the CAR-EC is a T cell, wherein the CAR-EC switch may act as an “on-switch” for CAR-EC activity. Activity may be “turned off” by reducing or ceasing administration of the switch. These CAR-EC switches may be used with CAR-ECs disclosed herein, as well as existing CAR T-cells, for the treatment of a disease or condition, such as cancer, wherein the target cell is a malignant cell. Such treatment may be referred to herein as switchable immunotherapy (US Patent Publication U.S. Pat. No. 9,624,276 B2 incorporated herein by reference in its entirety).

In some embodiments, the LNPs and methods of the disclosure are useful to modify effector cells (e.g., T cells) to express an mRNA encoding a receptor that binds the Fc portion of human immunoglobulins (e.g., CD16V-BB-ζ) (Kudo et al., (2014) Cancer Res 74(1):93-103 incorporated herein by reference in its entirety).

In some embodiments, the LNPs and methods of the disclosure are useful to modify effector cells (e.g., T cells) to express an mRNA encoding a universal immune receptor (e.g., switchable CAR, sCAR) that binds a peptide neo-epitope (PNE). In some embodiments, the peptide neo-epitope (PNE), has been incorporated at defined different locations within an antibody targeting an antigen (antibody switch). sCAR-T-cell specificity is redirected against the PNE, not occurring in the human proteome, thus allowing an orthogonal interaction between the sCAR-T-cell and the antibody switch. In this way, sCAR-T cells are strictly dependent on the presence of the antibody switch to become fully activated, thus excluding CAR T-cell off-target recognition of endogenous tissues or antigens in the absence of the antibody switch (Arcangeli et al., (2016) Transl Cancer Res 5(Suppl 2):S174-S177 incorporated herein by reference in its entirety). Other examples of switchable CARs is provided by US Patent Application US20160272718A1 incorporated herein by reference in its entirety.

### Uses of Lipid-Based Compositions

The present disclosure provides improved lipid-based compositions, in particular LNP compositions, with enhanced delivery of nucleic acids to immune cells. The present disclosure is based, at least in part, on the discovery that components of LNPs, act as immune cell delivery potentiating lipids that enhance delivery of an encapsulated nucleic acid molecule (e.g., an mRNA) to immune cells, such as lymphoid cells and myeloid cells (e.g., T cells, B cells, monocytes and dendritic cells).

The improved lipid-based compositions of the disclosure, in particular LNPs, are useful for a variety of purposes, both in vitro and in vivo, such as for nucleic acid delivery to immune cells, protein expression in or on immune cells, modulating immune cell (e.g., T cell, B cell, monocyte, and/or dendritic cell) activation or activity, increasing an immune response to a protein (e.g., infectious disease or cancer antigen) of interest (e.g., for vaccination or therapeutic purposes) and decreasing immune cell responses to reduce autoimmunity (e.g., to tolerize T cells).

For in vitro protein expression, the immune cell is contacted with the LNP by incubating the LNP and the immune cell ex vivo. Such immune cells may subsequently be introduced in vivo.

For in vivo protein expression, the immune cell is contacted with the LNP by administering the LNP to a subject to thereby increase or induce protein expression in or on immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the immune cell is contacted with the LNP by incubating the LNP and the immune cell ex vivo. In one embodiment, the immune cell is a human immune cell. In another embodiment, the immune cell is a primate immune cell. In another embodiment, the immune cell is a human or non-human primate immune cell. Various types of immune cells have been demonstrated to be transfectable by the LNP (see e.g., Examples 3, 5, 6, 9 and 10). In one embodiment, the immune cell is a T cell (e.g., a CD3+ T cell, a CD4+ T cell, a CD8+ T cell or a CD4+CD25+ CD127^{low} Treg cell). In one embodiment, the immune cell is a B cell (e.g., a CD19+ B cell). In one embodiment, the immune cell is a dendritic cell (e.g., a CD11c+CD11b− dendritic cell). In one embodiment, the immune cell is a monocyte/macrophage (e.g., a CD11c−CD11b+ monocyte/macrophage). In one embodiment, the immune cell is an immature NK cell (e.g., a CD56^{HIGH} immature NK cell). In one embodiment, the immune cell is an activated NK cell (e.g., a CD56^{DIM} activated NK cell). In one embodiment, the immune cell is an NK T cell (e.g., a CD3+CD56+ NK T cell). In one embodiment, the immune cell is an AML leukemia cell (e.g., a CD33+ AML cell). In one embodiment, the immune cell is a bone marrow plasma cell (e.g., CD45+CD38+CD138+CD19+CD20− bone marrow plasma cell).

In one embodiment, the immune cell is contacted with the LNP in the presence of serum or C1q for at least 15 minutes, which has been shown to be sufficient time for transfection of the cells ex vivo (see Example 13). In another embodiment, the immune cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the immune cell is contacted with the LNP for a single treatment/transfection. In another embodiment, the immune cell is contacted with the LNP for multiple treatments/transfections (e.g., two, three, four or more treatments/transfections of the same cells). Repeat transfection of the same cells has been demonstrated to lead to a dose-related increase in the percentage of cells transfected and in the level of expression of a protein encoded by the transfected nucleic acid without impacting cell viability (see Example 12).

In another embodiment, for in vivo delivery, the immune cell is contacted with the LNP by administering the LNP to a subject to thereby deliver the nucleic acid to immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, an intracellular concentration of the nucleic acid molecule in the immune cell is enhanced. In one embodiment, an activity of the nucleic acid molecule in the immune cell is enhanced. In one embodiment, expression of the nucleic acid molecule in the immune cell is enhanced. In on embodiment, the nucleic acid molecule modulates the activation or activity of the immune cell. In one embodiment, the nucleic acid molecule increases the activation or activity of the immune cell. In one embodiment, the nucleic acid molecule decreases the activation or activity of the immune cell.

In certain embodiments, delivery of a nucleic acid to an immune cell by the immune cell delivery potentiating lipid-containing LNP results in delivery to a detectable amount of immune cells (e.g., delivery to a certain percentage of immune cells), e.g., in vivo following administration to a subject. In some embodiments, the immune cell delivery potentiating lipid containing LNP does not include a targeting moiety for immune cells (e.g., does not include an antibody with specificity for an immune cell marker, or a receptor ligand which targets the LNP to immune cells). For example, in one embodiment, administration of the immune cell delivery potentiating lipid-containing LNP results in delivery of the nucleic acid to at least about 15% of splenic T cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 15%-25% of splenic B cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 35%-40% of splenic dendritic cells in vivo after a single intravenous injection (e.g., in a mouse model such as described in Example 15 or in a non-human primate model such as described in Example 8). In another embodiment, administration of the immune cell delivery potentiating lipid-containing LNP results in delivery of the nucleic acid to at least about 5%-20% of bone marrow cells (femur and/or humerus) in vivo after a single intravenous injection (e.g., in a mouse model such as described in Example 15 or in a non-human primate model such as described in Example 8). The levels of delivery demonstrated herein make in vivo immune therapy possible.

In one embodiment, uptake of the nucleic acid molecule by the immune cell is enhanced. Uptake can be determined by methods known to one of skill in the art. For example, association/binding and/or uptake/internalization may be assessed using a detectably labeled, such as fluorescently labeled, LNP and tracking the location of such LNP in or on immune cells following various periods of incubation. In addition, mathematical models, such as the ordinary differential equation (ODE)-based model described by Radu Mihaila, et al., (Molecular Therapy: Nucleic Acids, Vol. 7: 246-255, 2017; herein incorporated by reference), allow for quantitation of delivery and uptake.

In another embodiment, function or activity of a nucleic acid molecule can be used as an indication of the delivery of the nucleic acid molecule. For example, in the case of siRNA, reduction in protein expression in a certain proportion of immune cells can be measured to indicate delivery of the siRNA to that proportion of cells. Similarly, in the case of mRNA, increase in protein expression in a certain proportion of immune cells can be measured to indicate delivery of the siRNA to that proportion of cells. One of skill in the art will recognize various ways to measure delivery of other nucleic acid molecules to immune cells.

In certain embodiments, the nucleic acid delivered to the immune cell encodes a protein of interest. Accordingly, in one embodiment, an activity of a protein of interest encoded by the nucleic acid molecule in the immune cell is enhanced. In one embodiment, expression of a protein encoded by the nucleic acid molecule in the immune cell is enhanced. In one embodiment, the protein modulates the activation or activity of the immune cell. In one embodiment, the protein increases the activation or activity of the immune cell. In one embodiment, the protein decreases the activation or activity of the immune cell.

In one embodiment, various agents can be used to label cells (e.g., T cell, B cell, monocyte, or dendritic cell) to measure delivery to that specific immune cell population. For example, the LNP can encapsulate a reporter nucleic acid (e.g., an mRNA encoding a detectable reporter protein), wherein expression of the reporter nucleic acid results in labeling of the cell population to which the reporter nucleic acid is delivered. Non-limiting examples of detectable reporter proteins include enhanced green fluorescent protein (EGFP) and luciferase.

Delivery of the nucleic acid to the immune cell by the immune cell delivery potentiating lipid-containing LNP can be measured in vitro or in vivo by, for example, detecting expression of a protein encoded by the nucleic acid associated with/encapsulated by the LNP or by detecting an effect (e.g., a biological effect) mediated by the nucleic acid associated with/encapsulated by the LNP. For protein detection, the protein can be, for example, a cell surface protein that is detectable, for example, by immunofluorescence or flow cytometery using an antibody that specifically binds the cell surface protein. Alternatively, a reporter nucleic acid encoding a detectable reporter protein can be used and expression of the reporter protein can be measured by standard methods known in the art.

Methods of the disclosure are useful to deliver nucleic acid molecules to a variety of immune cell types, including normal immune cells and malignant immune cells. In one embodiment, the immune cell is selected from the group consisting of T cells, dendritic cells, monocytes and B cells.

The methods can be used to deliver nucleic acid to immune cells located, for example, in the spleen, in the peripheral blood and/or in the bone marrow. In one embodiment, the immune cell is an immune progenitor cell. In one embodiment, the immune cell is a T cell. T cells can be identified by expression of one or more T cell markers known in the art, typically CD3. Additional T cell markers include CD4 or CD8. In one embodiment, the immune cell is a B cell. B cells can be identified by expression of one or more B cell markers known in the art, typically CD19. Additional B cell markers include CD24 and CD72. In one embodiment, the immune cell is a monocyte and/or a tissue macrophage. Monocytes and/or macrophages can be identified by expression of one or more monocyte and/or macrophage markers known in the art, such as CD2, CD1 b, CD14 and/or CD16. In one embodiment, the immune cell is a dendritic cell. Dendritic cells can be identified by expression of one or more dendritic cell markers known in the art, typically CD11c. Additional dendritic cell markers include BDCA-1 and/or CD103.

The methods of the disclosure are useful to deliver nucleic acid molecules to bone marrow cells (e.g., in vivo), including immune cells within the bone marrow as well as other bone marrow cells, such as hematopoietic stem cells, immune cell precursers and fibroblasts. In one embodiment, the immune cells are bone marrow plasma cells. In one embodiment, the immune cells are bone marrow monocytes. In one embodiment, the cells are bone marrow early progenitor cells (e.g., proerythroblasts, monoblasts/myeloblasts and/or mesenchymal stem cells).

In one embodiment, the immune cell is a malignant cell, a cancer cell, e.g., as demonstrated by deregulated control of G1 progression. In one embodiment, the immune cell is a T cell that is malignant, cancerous or that exhibits deregulated control of G1 progression. In one embodiment, the immune cell is a leukemia cell or lymphoma cell. In one embodiment, the immune cell is an acute myelocytic leukemia (AML) cell. In other embodiments, the immune cell is a B cell that is malignant, e.g., a Hodgkins lymphoma cell, a non-Hodgkin's lymphoma cell, an anaplastic large cell lymphoma cell, a precursor-T lymphoblastic lymphoma, a follicular lymphoma cell, a small lymphocytic lymphoma cell, a marginal zone lymphoma cell, a diffuse large B cell lymphoma cell, a mantle cell lymphoma cell, a Burkitt's lymphoma cell, an acute lymphoblastic leukemia cell or a chronic lymphocytic leukemia cell.

The improved lipid-based compositions, including LNPs of the disclosure are useful to deliver more than one nucleic acid molecules to an immune cell or different populations of immune cells, by for example, administration of two or more different LNPs. In one embodiment, the method of the disclosure comprises contacting the immune cell (or administering to a subject), concurrently or consecutively, a first LNP and a second LNP, wherein the first and second LNP encapsulate the same or different nucleic acid molecules, wherein the first and second LNP include a phytosterol as a component. In other embodiments, the method of the disclosure comprises contacting the immune cell (or administering to a subject), concurrently or consecutively, a first LNP and a second LNP, wherein the first and second LNP encapsulate the same or different nucleic acid molecules, wherein the first LNP includes a phytosterol as a component and the second LNP lacks a phytosterol.

Myeloid Cells

Down Modulation of Dendritic and/or Myeloid Cell Activity

In one embodiment of the method for inhibiting immune responses, the nucleic acid associated with/encapsulated by the lipid-based compositions, including LNPs, of the disclosure is a nucleic acid that modulates (e.g., upregulates or downregulates) the activity of a dendritic cell or a myeloid cell such that an immune response is inhibited. The nucleic acid may itself cause modulation of dendritic or myeloid cell activity (e.g., such as siRNAs, mIRs and/or antagomirs) or can encode a protein that causes modulation of dendritic or myeloid cell activity (e.g., mRNA). Targeting of the nucleic acid to a dendritic cell or myeloid cell may directly result in effects that lead to inhibition of immune responses or, alternatively, targeting of the nucleic acid to the dendritic or myeloid cell may result in effects that thereby modulate the activity of one of more other cell type(s) (e.g., T cell, B cells) that then leads to inhibition of immune responses. For example, in some aspects, targeting of a nucleic acid to a dendritic or myeloid cell leads to effects that modulate regulatory T cell activity or stimulates a response (e.g., cytokine production) that modulates other immune cells such that an immune response is inhibited.

In one embodiment, the nucleic acid encodes or modulates (e.g., upregulates or downregulates) the activity of a naturally-occurring molecule. In one embodiment, the naturally-occurring molecule is an intracellular molecule, such as a transcription factor or cell signaling cascade molecule. In another embodiment, the naturally-occurring molecule is a secreted molecule, such as a cytokine or a chemokine. In another embodiment, the naturally-occurring molecule is a transmembrane molecule, such as surface receptors, homing molecules, signaling molecules or immune modulation molecules (e.g., HLA molecules).

In one embodiment, the nucleic acid encodes a modified (i.e., non-naturally-occurring) molecule. Non-limiting examples of modified proteins include proteins having an altered (e.g., increased or decreased) half-life and proteins modified such that they tethered to a cell surface (e.g., addition of a transmembrane domain to a soluble protein such that the modified protein tethers the soluble protein to a cell surface).

In another embodiment, the nucleic acid encodes a modified (i.e., non-naturally-occurring) transmembrane or intracellular protein. Non-limiting examples of modified transmembrane proteins include chimeric receptors, dominant-negative receptors and constitutively-active mutant receptors. Non-limiting examples of modified intracellular proteins include mutant/altered enzymes (e.g., kinases, phosphatases, dioxygenases and the like) that participate in signaling cascades, including molecules that can alter (e.g., invert) the signaling at an immune synapse (e.g., agonize or antagonize signaling through T cell receptors (TCR) or B cell receptors (BCR)), as well as molecules that can modulate (e.g., induce) apoptosis.

Non-limiting examples of specific approaches for modulating dendritic cell and/or myeloid cell activity to thereby inhibit immune responses are described in further detail below.

### Induction of Tolerogenic Dendritic Cells

In one embodiment of the method for inhibiting immune responses, the nucleic acid associated with/encapsulated by the lipid-based compositions, including LNPs, of the disclosure is a nucleic acid that stimulates (e.g., promotes, enhances, upregulates) the induction of tolerogenic dendritic cells (tolDC) such that an immune response is inhibited (e.g., a nucleic acid that encodes a protein that stimulates the induction of tolDC). Dendritic cells (DC) are key regulators of T and B cell immune responses and are capable of inducing tolerance. Several intracellular networks that program DCs to become tolerogenic have been identified. For example, tolerogenic signals, such as TGFβ, IL-10 or PGE, applied to immature DCs stimulate the development of regulatory DCs, in which signaling pathways mediated by, for example, IL-10, TGFβ, IDO, PD-1 and/or ARG, can induce tolerogenic DCs that can stimulate the generation of regulatory T (Treg) cells and/or Type I regulatory T (Tr1) cells, as well as inhibit the proliferation of conventional CD4+ T cells and/or CD8+ T cells. In particular, overlapping signaling network nodes can be leveraged to induce tolDC. Non-limiting examples of specific approaches for inducing tolerogenic DCs to thereby inhibit immune responses are described in further detail below.

(i) NFkB Inhibition

The maturation of DCs into cells that mediate effector T cell activation and differentiation, as well as produce proinflammatory cytokines, has been established in the art as being regulated by the transcription factor NFkB. Accordingly, inhibition of NFkB activity has been describe in the art as an approach for inducing DC tolerogenicity and inhibiting immune response in autoimmune diseases. For example, inhibition of NFkB inducing kinase (NIK) has been shown to be an effective treatment for experimental lupus (Brightbill et al. (2018) Nature Commun. 9:179). Additionally, DEN-181, a lipid nanoparticle-based immunotherapy that comprises an NFkB inhibitor has entered Phase I trials for rheumatoid arthritis.

Accordingly, in one embodiment, the LNP of the disclosure provides a nucleic acid that induces tolerogenic DCs by inhibiting NFkB activity, directly or indirectly. Non-limiting examples of pathways that may be targeted to interfere with NFkB activity in dendritic cells include those mediated by PPARg, STAT3, GILZ, GR, SHP1/2, HO1, A20, SOCS1, SOCS2, AMPK and AhR. Accordingly, in some aspects of the disclosure, tolerogenic nucleic acids (e.g., mRNAs) that suppress NFkB activation are used to induce tolDC. Non-limiting examples of tolerogenic mRNAs that suppress NFkB activity include those encoding SOCS1, SOCS2, AMPKa1, AMPKa2, CAMKK2, LKB1, FOXO3, PPARg, HMOX1, GILZ and STAT3.

(ii) Modulation of Amino Acid Metabolism

In another embodiment, the LNPs of the disclosure provide a nucleic acid that induces tolerogenic DCs by modulating amino acid metabolism. Over the course of immune system evolution, two primary amino acid metabolic regulatory nodes were selected: arginine and tryptophan. The regulatory enzymes that metabolize arginine are inducible nitric oxide synthase (iNOS), arginase-1 (Arg1) and arginase-2 (Arg2), whereas the regulatory enzymes that metabolize tryptophan are indoleamine2, 3-dioxygenases (IDO1 and IDO2) and tryptophan-2, 3-dioxygenase (TDO).

Tryptophan is catabolized by IDO and TDO into immunosuppressive kynurenines (KYN), which stimulate Treg induction. A key target of KYN in Treg cells is the aryl hydrocarbon receptor (AhR), a ligand-activated transcription factor involved in regulating immune responses (see e.g., Stockinger et al. (2014) Ann. Rev. Immunol. 32:403-432; Gutierrez-Vazquez et al. (2015) Immunity 48:19-33), including the suppression of inflammation (Crunkhorn (2018) Nature Rev. Drug. Disc. 17:470). Accordingly, in some aspects, the disclosure provides nucleic acids (e.g., mRNAs) that modulate tryptophan metabolism to thereby induce tolDC. Non-limiting examples include nucleic acids that stimulate or encode IDO1, IDO2, TDO or AhR.

Arginine metabolism also plays a role in the ability of dendritic cells to induce tolerance. Downregulation of arginine metabolism in dendritic cells using inhibitors of iNOS and arginase has been shown to induce tolerance to exogenous antigen (Simioni et al. (2017) Int. J. Immunopathol. Pharmacol. 30:44-57). Accordingly, in some aspects, the disclosure provides nucleic acids (e.g., mRNAs) that modulate arginine metabolism to thereby induce tolDC. Non-limiting examples include nucleic acids that inhibit (e.g., an inhibitor) or encode iNOS, Arg1 or Arg2.

(iii) Activation of Wnt/Beta Catenin Pathway

The Wnt/beta catenin pathway plays an important role in the regulation of DC activity. Wnts are secreted lipid-modified glycoproteins that bind to Frizzled (FZD) receptors and activate multiple signaling pathways. Certain Wnts (e.g., Wnt3a, Wnt5b, Wnt16) activate the canonical beta catenin/TCF pathway in DCs, whereas other Wnts (e.g., Wnt5a) activate the non-canonical beta catenin-independent pathway. Low-density lipoprotein receptor-related protein 5 (LRP5) and LRP6 co-receptors are critical signaling mediators of the canonical Wnt-signaling pathway. Wnt ligand interaction with FZD and co-receptors LRP5/6 results in the activation of beta catenin in the cytoplasm and its translocation to the nucleus, where it interacts with T-cell factor/lymphoid enhancer factor (TCF/LEF) family members and regulates the transcription of various target genes. For reviews of Wnt/beta catenin pathway signaling in DC cells, and effects thereof, see e.g., Swafford et al. (2015) Discov. Med. 19:303-310 and Suryawanshi et al (2016) Front. Immunol. 7:460.

Wnt-mediated activation of the canonical beta catenin pathway in DCs has been shown to bias the DCs to a tolerogenic phenotype and induce the expression of immunosuppressive (e.g., anti-inflammatory) genes. For example, Wnt3a has been shown to trigger canonical beta catenin signaling in DCs and stimulate the production of anti-inflammatory cytokines, such as TGF-beta and VEGF (Oderup et al. (2013) J. Immunol. 190:6126-6134). Wnt16b has been shown to induce differentiation of regulatory T cells through beta catenin signaling in DCs (Shen et al. 2014) Int. J. Mol. Sci. 15:12928-12939). Canonical Wnt signaling in DCs also has been shown to regulate Th1/Th17 responses and to suppress autoimmune neuroinflammation (Suryawanshi et al. (2015) J. Immunol. 194:3295-3304). Still further, activation of the beta catenin pathway in dendritic cells has been shown to be a key regulator of mucosal tolerance in the intestines (Manicassamy et al. (2010) Science 329:849-853). Accordingly, in some aspects, the disclosure provides nucleic acids (e.g., mRNAs) that modulate (e.g., activate) the Wnt-mediated beta catenin pathway in DCs to thereby induce tolDC. Non-limiting examples include mRNAs encoding Wnt3a, Wnt16b, constitutively-active beta catenin, constitutively-active LRP5 and constitutively-active LRP 6.

Wnt-mediated activation of the non-canonical beta catenin-independent pathway in DCs also has been shown to bias the DCs to a tolerogenic phenotype and to induce the expression of immunosuppressive (e.g., anti-inflammatory) genes. For example, Wnt5a has been shown to skew dendritic cell differentiation to an unconventional phenotype with tolerogenic features (Valencia et al. (2011) J. Immunol. 187:4129-4139). Additionally, Wnt5a has been shown to induce the expression of the anti-inflammatory cytokine IL-10 in DCs, as well as inhibit the production of the pro-inflammatory cytokine IL-6 (Oderup et al. (2013) J. Immunol. 190:6126-6134). Accordingly, in some aspects, the disclosure provides nucleic acids (e.g., mRNAs) that modulate (e.g., activate) the Wnt-mediated non-canonical beta catenin-independent pathway in DCs to thereby induce tolDC. Non-limiting examples include mRNAs encoding Wnt5a.

Still further, Wnt-independent activation of the beta catenin pathway in DCs has been shown to program DCs to a tolerogenic state. For example, disruption of E-cadherin-mediated adhesion in DCs has been shown to activate beta catenin signaling, skewing the DCs to a tolerogenic phenotype, including production of high levels of the anti-inflammatory cytokine IL-10 and the ability to protect mice against experimental autoimmune encephalitis (Jiang et al. (2007) Immunity 27:610-624). Furthermore, TLR2-mediated signaling has been shown to activate beta catenin in DCs, leading to the production of IL-10 and promotion of Treg cell differentiation (Manoharan et al. (2014) J. Immunol. 193:4203-4213; Manicassamy et al. (2010) Science 329:849-853). Moreover, additional signaling pathways have been shown to activate or regulate beta catenin in DCs and regulate adaptive immunity, including TLR3 (Gantner et al. (2012) J. Immunol. 189:3104-3111), TLR9 (Manoharan et al. (2014) J. Immunol. 193:4203-4213), FAS (Qian et al. (2013) J. Biol. Chem. 288:27825-27835), TGF-β (Vander Lugt et al. (2011) PLoS One 6(5):e20099) and PLC-γ2 (Capietto et al. (2013) J. Exp. Med. 210:2257-2271). Accordingly, in some aspects, the disclosure provides nucleic acids (e.g., mRNAs) that modulate (e.g., activate) Wnt-independent beta catenin signaling in DCs to thereby induce tolDC. Non-limiting examples include mRNAs encoding TLR2, TLR3, TLR9, FAS, TGF-β, PLC-γ2 and E-cadherin inhibitors.

(iv) Activation of TGF-β/SMAD Signaling

Transforming growth factor beta (TGF-β) is a pleiotropic cytokine that has been established in the art to play a pivotal role in the induction of immunological tolerance in DCs. TGF-β has been determined to function as an immune suppressor by influencing the development, differentiation, tolerance induction and homeostatis of immune cells, including dendritic cells. For reviews of the role of TGF-β in dendritic cells, see e.g., Esebanmen et al. (2017) Immunol. Res. 65:987-994; Sanjabi et al. (2017) Cold Spring Harbor Perspective Biol. 9(6); Seeger et al. (2015) Cytokine Growth Factor Rev. 26:647-657; Sheng et al. (2015) Growth Factors 33:92-101. TGF-β has been shown to downregulate the antigen-presenting function and expression of co-stimulatory molecules by DCs in vitro (Strobl and Knapp (1999) Microbes Infect. 1:1283-1290). Moreover, TGF-β signaling in dendritic cells has been shown to be a prerequisite for the control of autoimmune encephalitis (Laouar et al. (2008) Proc. Natl. Acad. Sci. USA 105:10865-10870). The main signal transducers for the receptors of the TGF-β family are SMADS, including SMAD1, SMAD2, SMAD3, SMAD5 and SMAD8/9. Accordingly, in some aspects, the disclosure provides nucleic acids (e.g., mRNAs) that activate TGF-β/SMAD signaling to thereby induce tolDC. Non-limiting examples include mRNAs that encode constitutively-active TGF-β (e.g., constitutively active TGF-β1), TGF-β receptor, SMAD1, SMAD2, SMAD3, SMAD5 or SMAD8/9.

(v) Activation of IL-10/STAT3 Signaling

The cytokine interleukin-10 (IL-10) has been established as an important negative regulator of DC activation (for a review, see e.g., Ma et al. (2015) F1000 Research 4:1465). IL-10 acts as a tolerogenic signal on immature dendritic cells to bias them toward differentiation to tolerogenic DCs. The effects of IL-10 in DCs are mediated through the transcription factor STAT3 (see e.g., Melillo et al. (2010) J. Immunol. 184:2638-2645). Accordingly, in some aspects, the disclosure provides nucleic acids (e.g., mRNAs) that activate IL-10/STAT3 signaling to thereby induce tolDC. Non-limiting examples include mRNAs that encode IL-10 or STAT3 (e.g., constitutively active STAT3).

(vi) PD-L1 Expression

Dendritic cells have been shown to induce T cell tolerance through stimulation of the PD-1 coinhibitory receptor on T cells via expression of the PD-1 ligand PD-L1 on the surface of the dendritic cells (see e.g., Probst et al. (2005) Nat. Immunol. 6:280-286; Sage et al. (2018) J. Immunol. 200:2592-2602). Accordingly, in some aspects, the disclosure provides nucleic acids (e.g., mRNAs) that encode PD-L1 or stimulate the expression or activity of PD-L1 on DC cell surface to thereby induce tolDC.

(vii) Modulation of PPARγ Activity

Peroxisome proliferator-activated receptor gamma (PPARγ) is nuclear receptor that has been implicated in both upregulation and downregulation of the tolerogenic effects of DCs. For example, PPARγ has been demonstrated to down-regulate allergic inflammation and eosinophil activation (Woerly et al. (2003) J. Exp. Med. 198:411-421).

Additionally, in other studies, activation of PPARγ in dendritic cells has been shown to inhibit the development of eosinophilic airway inflammation in a mouse model of asthma (Hammad et al. (2004) Am J. Pathol. 164:263-271). More recently, expression of PPARγ in dendritic cells has been shown to play a role in the development of type-2 immune responses, thereby indicating a proinflammatory role for PPARγ in dendritic cells in certain instances (Nobs et al. (2017) J. Exp. Med. 214:3015-3035). Accordingly, in some aspects, the disclosure provides nucleic acids (e.g., mRNAs) that encode PPARγ or modulate (e.g., upregulate or downregulate) the expression or activity of PPARγ in DCs to thereby induce tolDC.

(viii) Activation of AMPK

5′ AMP-activated protein kinase (AMPK) is an enzyme that plays a role in cellular energy homeostatis, including glucose activation and fatty acid uptake and oxidation. Pharmacological activation of AMPK has been shown to suppress TLR-induced glucose consumption and activation of dendritic cells and systemic administration of drugs activating AMPK signaling has been shown to drive induction of tolerogenic immune responses in several inflammatory disease models (reviewed in Everts and Pearce (2014) Front. Immunol. 5:203). Accordingly, in some aspects, the disclosure provides nucleic acids (e.g., mRNAs) that encode AMPK or activate the expression or activity of AMPK in DCs to thereby induce tolDC.

Regulation of Treg Cells by Dendritic and Myeloid Cells

In one embodiment of the method for inhibiting immune responses, the nucleic acid associated with/encapsulated by the lipid-based compositions, including LNPs, of the disclosure is a nucleic acid that functions to regulate Treg cells, such as induce, expand and/or or maintain Treg cells, such that an immune response is inhibited (e.g., an mRNA that encodes a protein that functions to induce, expand and/or maintain Treg cells). In one embodiment, the nucleic acid that functions to induce, expand and/or maintain Treg cells is expressed in dendritic cells and/or myeloid cells such that Treg cells are induced, expanded and/or maintained. In one embodiment, the nucleic acid that functions to induce, expand and/or maintain Treg cells that is expressed in dendritic cells and/or myeloid cells is a Treg-inducing cytokine.

Numerous cytokines have been described in the art that have been demonstrated to be involved in inducing, expanding and/or maintaining Treg cells. For example, Treg cells express an abundance of IL-2 receptor but are unable to produce IL-2 themselves. Accordingly, Treg cells rely on IL-2 produced by other cells. Thus, in one embodiment, the disclosure provides a nucleic acid that encodes IL-2 (e.g., mRNA encoding IL-2) which is expressed by dendritic and/or myeloid cells to thereby induce, expand and/or maintain Treg cells.

In addition to IL-2, TGF-β has been demonstrated to promote the induction of Treg, including in combination with IL-2 (see e.g., Lu et al. (2010) PLoS One 5:e15150; Chen et al. (2011) J. Immunol. 186:6329-6337). Accordingly, in one embodiment, the disclosure provides a nucleic acid (e.g., mRNA) that encodes TFG-β, alone or in combination with a nucleic acid (e.g., mRNA) that encodes IL-2, which is expressed in dendritic and/or myeloid cells to thereby induce, expand and/or maintain Treg cells.

The anti-inflammatory cytokine IL-10 has been shown to promote the generation of Treg cells (see e.g., Heo et al. (2010) Immunol. Letters 127:150-156). IL-10 also has been demonstrated to endow Treg cells with the ability to suppress pathogenic Th17 cell response (Chaudhry et al. (2011) Immunity 34:566-578). Furthermore, IL-10 potentiates the differentiation of induced Treg cells (iTreg) (Hsu et al. (2015) J. Immunol. 195:3665-3674). Accordingly, in one embodiment, the disclosure provides a nucleic acid (e.g., mRNA) that encodes IL-10, alone or in combination with other cytokine-encoding mRNAs (e.g., IL-2, TFG-13) which is expressed in dendritic and/or myeloid cells to thereby induce, expand and/or maintain Treg cells.

Treatment of naïve human T cells with IL-35 has been demonstrated to induce a regulatory T cell population, referred to as iT_{R}35 regulatory cells, that mediates immunosuppression via IL-35 but not by IL-10 or TFG-β (Collison et al. (2010) Nat. Immunol. 11:1093-1101). These iT_{R}35 regulatory cells do not express or require the transcription factor Foxp3 and are strongly suppressive and stable in vivo (Collison, supra). Human Tregs, however, do not constitutively express IL-35 (Bardel et al. (2008) J. Immunol. 181:6898-6905). Accordingly, iT_{R}35 regulatory cells rely on IL-35 produced by other cells. Thus, in one embodiment, the disclosure provides a nucleic acid that encodes IL-35 (e.g., mRNA encoding IL-35) which is expressed in dendritic and/or myeloid cells to thereby induce, expand and/or maintain Treg cells (e.g., iT_{R}35 regulatory cells).

GM-CSF treatment has been shown to lead to dendritic cell expansion and an accumulation of Treg cells, which has been demonstrated to inhibit autoimmunity in a variety of in vivo models, including diabetes (Gaudreau et al. (2007) J. Immunol. 179:3638-3647), myasthenia gravis (Sheng et al. (2008) Clin. Immunol. 128:172-180) and autoimmune thyroiditis (Ganesh et al. (2009) Int. Immunol. 21:269-282). Accordingly, in some aspects, the disclosure provides a nucleic acid that encodes GM-CSF (e.g., mRNA encoding GM-CSF) which is expressed in dendritic and/or myeloid cells to thereby induce, expand and/or maintain Treg cells.

Up Modulation of Dendritic and/or Myeloid Cell Activity

In one embodiment, the immune cell delivery potentiating lipid compositions of the disclosure are used to stimulate (upregulate, enhance) the activation or activity of an immune cell, such as a dendritic or myeloid cell, for example in situations where stimulation of an immune response is desirable, such as in cancer therapy or treatment of an infectious disease (e.g., a viral, bacterial, fungal, protozoal or parasitic infection).

In one embodiment of stimulating the activation or activity of an immune cell, such as a dendritic cell or myeloid cell, the agent (e.g., mRNA) associated with/encapsulated by the lipid nanoparticle encodes a protein that is a cytokine, non-limiting examples of which include the cytokines described in the soluble targets section herein.

In one embodiment of stimulating the activation or activity of an immune cell, such as a dendritic cell or myeloid cell, the agent (e.g., mRNA) associated with/encapsulated by the lipid nanoparticle encodes a protein that is a chemokine or a chemokine receptor, non-limiting examples of which are described in the soluble targets section herein.

In one embodiment of stimulating the activation or activity of an immune cell, such as a dendritic cell or myeloid cell, the agent (e.g., mRNA) associated with/encapsulated by the lipid nanoparticle encodes a protein that is a costimulatory factor that upregulates immune responses or is an antagonist of a costimulatory factor that downregulates immune responses, non-limiting examples of which are described herein.

In one embodiment, of stimulating the activation or activity of an immune cell, such as a dendritic cell or myeloid cell, the agent (e.g., mRNA) associated with/encapsulated by the lipid nanoparticle encodes a protein is an antigen, such as a vaccine antigen (e.g, viral antigen, bacterial antigen, tumor antigen). For example, as described in Example 14, the lipid-based compositions of the invention have been demonstrated to enhance the immunogenicity of an mRNA vaccine encoding a viral antigen. Accordingly, in one embodiment, the agent (e.g., mRNA) associated with/encapsulated by the lipid nanoparticle encodes an antigen of interest, such as a cancer antigen or an infectious disease antigen (e.g., a bacterial antigen, a viral antigen, a fungal antigen, a protozoa antigen or a parasite antigen).

Lymphoid Cells

### Modulation of B Cell Activity

In one embodiment of the method for inhibiting immune responses, the nucleic acid associated with/encapsulated by the lipid-based compositions, including LNPs, of the disclosure is a nucleic acid that modulates (e.g., upregulates or downregulates) the activity of a B cell such that an immune response is inhibited. The nucleic acid may itself cause modulation of B cell activity (e.g., such as antisense nucleic acids, siRNAs, mIRs and/or antagomirs) or encodes a protein that causes modulation of B cell activity (e.g., mRNA). Targeting of the nucleic acid to a B cell may directly result in effects that lead to inhibition of immune responses (e.g., downregulation of antibody production by the B cell). Alternatively, targeting of the nucleic acid to the B cell may result in effects that thereby modulate the activity of one of more other cell type(s) (e.g., other effector cells) that then leads to inhibition of immune responses. Furthermore, targeting of the nucleic acid to a non-B cell (e.g., to a dendritic cell, myeloid cell or T cell) may result in effects that lead to inhibition of B cell activity such that B cell-mediated immune responses are inhibited, such as downregulation of antibody production.

In one embodiment, the nucleic acid encodes or modulates (e.g., upregulates or downregulates) the activity of a naturally-occurring molecule. In one embodiment, the naturally-occurring molecule is an intracellular molecule, such as a transcription factor or cell signaling cascade molecule. In another embodiment, the naturally-occurring molecule is a secreted molecule, such as a cytokine or a chemokine. In another embodiment, the naturally-occurring molecule is a transmembrane molecule, such as surface receptors, homing molecules, signaling molecules or immune modulation molecules (e.g., HLA molecules).

In one embodiment, the nucleic acid encodes a modified (i.e., non-naturally-occurring) molecule. Non-limiting examples of modified proteins include proteins having an altered (e.g., increased or decreased) half-life and proteins modified such that they tethered to a cell surface (e.g., addition of a transmembrane domain to a soluble protein such that the modified protein tethers the soluble protein to a cell surface).

In another embodiment, the nucleic acid encodes a modified (i.e., non-naturally-occurring) transmembrane or intracellular protein. Non-limiting examples of modified transmembrane proteins include chimeric receptors, dominant-negative receptors and constitutively-active mutant receptors. Non-limiting examples of modified intracellular proteins include mutant/altered enzymes (e.g., kinases, phosphatases, dioxygenases and the like) that participate in signaling cascades, including molecules that alter (e.g., invert) the signaling at an immune synapse (e.g., agonize or antagonize signaling through T cell receptors (TCR) or B cell receptors (BCR)), as well as molecules that modulate (e.g., induce) apoptosis.

Non-limiting examples of specific approaches for modulating B cell activity to thereby inhibit immune responses are described in further detail below.

B Cell Depletion

Depletion of B cells has been described as a successful approach for inhibiting immune responses, e.g., in autoimmune diseases (reviewed in, for example, Sanz et al. (2007) Front. Biosci. 12:2546-2567; Dorner et al. (2009) Autoimmun. Rev. 9:82-89; Pateinakis et al. (2014) Biomed. Res. Intl. 2014:Article ID 973609). Accordingly, in one embodiment of the method for inhibiting immune responses, the nucleic acid associated with/encapsulated by the lipid-based compositions, including LNPs, of the disclosure is a nucleic acid that functions to deplete B cells, such that an immune response is inhibited (e.g., an mRNA that encodes a protein that functions to deplete B cells). In one embodiment, the nucleic acid is delivered to B cells themselves to thereby deplete the B cells (referred to a direct depletion). Additionally or alternatively, in certain embodiments, the nucleic acid is delivered to non-B cells (e.g., dendritic cells, myeloid cells, T cells) such that B cells are depleted (referred to as indirect depletion). In one embodiment, B cell depletion, either directly or indirectly, results in downregulation (i.e., inhibition, reduction, suppression) of antibody production (e.g., antigen-specific antibody production involved in an autoimmune reaction).

In one embodiment, the nucleic acid for depleting B cells encodes a B cell-specific antibody or targets a B cell surface marker signaling pathway. B cell-specific antibodies have been used in the art to achieve B cell depletion in vivo, including in the treatment of a variety of autoimmune diseases. For example, anti-CD20 antibodies (e.g., rituximab, ocrelizumab) have been used for B cell depletion in the treatment of, for example, rheumatoid arthritis (see e.g., Pavelka et al. (2005) Arthr. Rheum. 52:S131; Cohen et al. (2005) Arthr. Rheum. 52:S677; Keystone et al. (2005) Arthr. Rheum. 52:S 141); systemic lupus erythematosus (SLE) (see e.g., Leandro et al. (2005) Rheumatol. 44:1542-1545; Anolik et al. (2004) Arthr. Rheum. 50:3580-3590; Sfikakis et al. (2005) Arthr. Rheum. 52:501-513); vasculitis (Keogh et al. (2005) Arthr. Rheum. 52:262-268; Eriksson (2005) J. Inter. Med. 257:540-548; Guillevin et al. (2014) N. Engl. J. Med. 371:1771-1780); pemphigus (see e.g., Joly et al. (2017) Lancet 389:2031-2040); and multiple sclerosis (Hauser et al. (2017) N. Engl. J. Med. 376:221-234; Montalban et al. (2017) N. Engl. J. Med. 376:209-220). Accordingly, in one embodiment, the nucleic acid (e.g., mRNA) that depletes B cells is a nucleic acid encoding an anti-CD20 antibody or a nucleic acid which targets the CD20 signaling pathway in B cells.

Anti-CD19 antibodies (e.g., inebilizumab) also have been used to achieve B cell depletion in vivo, for example in the treatment of multiple sclerosis (see e.g., Agius et al. (2017) Mult. Scler. doi:10.1177/1352458517740641). Accordingly, in one embodiment, the nucleic acid (e.g., mRNA) that depletes B cells is a nucleic acid encoding an anti-CD19 antibody or a nucleic acid which targets the CD19 signaling pathway in B cells.

Anti-CD22 antibodies (e.g., epratuzumab) also have been used to achieve B cell depletion in vivo, for example in the treatment of SLE (see e.g., Wallace et al. (2014) Ann. Rheum. Dis. 73:183-190). Accordingly, in one embodiment, the nucleic acid (e.g., mRNA) that depletes B cells is a nucleic acid encoding an anti-CD22 antibody or a nucleic acid which targets the CD22 signaling pathway in B cells.

Antibodies against BAFF (B cell activating factor; also known in the art as TALL-1, THANK, BlyS and zTNF4) (e.g., belimumab) also have been used to achieve B cell depletion in vivo (see e.g., Rauch et al. (2009) PLoS One 4(5):e5456; Kowalczyk-Quintas et al. (2016) J. Biol. Chem. 291:18826-18834), including in the treatment of SLE (see e.g., Jacobi et al. (2010) Arthr. Rheum. 62:201-210). Accordingly, in one embodiment, the nucleic acid (e.g., mRNA) that is to deplete B cells is a nucleic acid encoding an anti-BAFF antibody or a nucleic acid which targets the BAFF signaling pathway in B cells.

Another approach for depletion of B cells is through activation of one or more regulatory pathways in the B cells that results in apoptosis of the B cells. Accordingly, in one embodiment, the nucleic acid for depleting B cells induces apoptosis in the B cells. For example, signaling through the inhibitory FcgRIIB receptor on B cells independent of antigen (e.g., though overexpression of the receptor or ligation of the receptor independent of antigen) has been shown to lead to apoptosis of the B cells (see e.g., Yang et al. (2009) Blood 114:3736; Tzeng et al. (2015) J. Biomed. Sci. 22:87). Accordingly, in one embodiment, the nucleic acid (e.g., mRNA) that is used for B cell depletion encodes the FcgRIIB receptor such that FcgRIIB is overexpressed in B cells as means to induce apoptosis in the B cells, thereby resulting in B cell depletion.

Another approach for depleting B cells through apoptosis is through the use of an antagonist of Mcl-1. The Mcl-1 protein is a pro-survival member of the Bcl-2 family of proteins. Mcl-1 blocks activation of the pro-apoptotic proteins Bax and Bak and has been reported to be critical for the survival and maintenance of multiple hematopoietic cell types (Anstee et al. (2017) Cell Death Diff. 24:397-408). Overexpression of Mcl-1 has been shown to exacerbate autoimmune kidney disease in mice (Anstee et al., supra). Moreover, Mcl-1 has been shown to be essential for the survival of plasma cells (Peperzak et al. (2013) Nat. Immunol. 14:290-297). Accordingly, in one embodiment, the nucleic acid that is used for B cell depletion is or encodes (e.g., mRNA) an Mcl-1 antagonist such that Mcl-1 is downregulated in B cells as means to induce apoptosis in the B cells, thereby resulting in B cell depletion. The Mcl-1 antagonist can be, for example, an inhibitory RNA (e.g., antisense RNA, siRNA, miRs). In one embodiment, the Mcl-1 antagonist is a microRNA (miR). mir-29 has been reported to regulate Mcl-1 protein expression and apoptosis, with enforced mir-29b expression leading to reduced Mcl-1 cellular protein expression and sensitization to TRAIL-mediated apoptosis (Mott et al. (2007) Oncogene 26:6133-6140). Accordingly, in one embodiment, the Mcl-1 antagonist nucleic acid is a member of the mir-29 family, such as mir-29b.

CAR-T cells also can be used for the purpose of depletion of B cells to thereby inhibit immune responses (e.g., in autoimmune diseases). For example, a CAR-T cell expressing a chimeric antigen receptor comprising truncated fragments of the Dsg3 extracellular domain fused to CD137/CD3 signaling domains has been used to deplete anti-Dsg3-specific B cells in vitro and in a pemphigus mouse model (see e.g., Ellebrecht et al. (2016) Science 353:179-184). Accordingly, in one embodiment, the nucleic acid (e.g., mRNA) that is used for B cell depletion encodes a CAR specific for a B cell (e.g., specific for an auto-antibody expressed on the surface of the B cell, or for a B cell marker expressed on the surface of the B cell, such as CD19, CD20 or CD22), such that expression of the CAR by a CAR-T cell results in B cell depletion (indirect B cell depletion).

Antigen Tolerization

Antigen-specific tolerization has been achieved in vivo through delivery of tolerogenic forms of antigens (e.g., tolerogenic forms of autoantigens), either alone or in combination with additional immune modifiers. In particular, autologous cells and nanoparticles have been used successfully to deliver tolerogenic antigens to achieve antigen-specific tolerization (reviewed in, for example, Pearson et al. (2017) Adv. Drug Deliv. Rev. 114:240-255; Kishimoto and Maldonado (2018) Front. Immunol. 9:230). Accordingly, in one embodiment of the method for inhibiting immune responses, the nucleic acid associated with/encapsulated by the lipid-based compositions, including LNPs, of the disclosure is a nucleic acid that functions to achieve antigen-specific tolerization, such that an immune response is inhibited (e.g., an mRNA that encodes a protein that functions to achieve antigen-specific tolerization). In one embodiment, the nucleic acid is delivered to B cells themselves to achieve antigen-specific tolerization. Additionally or alternatively, in certain embodiments, the nucleic acid is delivered to non-B cells (e.g., dendritic cells, myeloid cells, T cells) to achieve antigen-specific tolerization. In some aspects, the nucleic acid is delivered to an antigen presenting cell (e.g., dendritic cell, monocyte, macrophage) to thereby achieve antigen-specific tolerization of B cells.

In one embodiment, the nucleic acid (e.g., mRNA) used to achieve antigen tolerization encodes one or more tolerogenic antigens (e.g., one or more tolerogenic autoantigens). For example, microparticles carrying encephalitogenic peptides have been shown to induce tolerance and ameliorate experimental autoimmune encephalomyelitis (EAE) (Getts et al. (2012) Nat. Biotechnol. 30:1217-1224). Autologous myelin peptide-coupled cells have been shown to induce antigen-specific tolerance in multiple sclerosis (Lutterotti et al. (2013) Sci. Transl. Med. 5:188).

In another, embodiment, antigen tolerization is achieved using a nucleic acid that encodes one or more tolerogenic antigens (e.g., one or more tolerogenic autoantigens) in combination with one or more additional agents that promote-antigen specific tolerization, non-limiting examples of which include immune suppressants, inhibitory cytokines and immune modifiers. For example, rapamycin has been used in combination with tolerogenic antigens to achieve antigen-specific immunological tolerance in EAE (Maldonando et al. (2014) Proc. Natl. Acad. Sci. USA 112:E156-165). Furthermore, a small molecule activator of the aryl hydrocarbon receptor (AhR) has been used in combination with tolerogenic antigens to achieve antigen-specific tolerance in EAE (Yeste et al. (2012) Proc. Natl. Acad. Sci. USA 109:11270-11275). Still further, the cytokine IL-10 has been used in combination with tolerogenic antigens to achieve antigen-specific immunological tolerance in EAE (Cappellano et al. (2014) Vaccine 32:5681-5689). Accordingly, in some aspects, antigen tolerization is achieved using a nucleic acid encoding one or more tolerogenic antigens in combination with, for example, rapamycin, an AhR inhibitor or IL-10.

Suppression of Antibody Production

In one embodiment of the method for inhibiting immune responses, the nucleic acid associated with/encapsulated by the lipid-based compositions, including LNPs, of the disclosure is a nucleic acid that functions to suppress (i.e., inhibit, downregulate) antibody production, such that an immune response is inhibited (e.g., an mRNA that encodes a protein that functions to suppress antibody production). In one embodiment, the nucleic acid is delivered to B cells themselves to thereby suppress antibody production (referred to a direct suppression of antibody production). Additionally or alternatively, in some embodiments, the nucleic acid is delivered to non-B cells (e.g., T cells that regulate antibody-producing B cells) such that antibody production is suppressed (referred to as indirect suppression of antibody production).

In one embodiment, antibody production is directly suppressed in a B cell using a nucleic acid that suppresses or inhibits the transcription or translation of antibody genes in the B cell. For example, an inhibitory RNA (e.g., siRNA, antisense RNA, miR) is used that targets the antibody heavy or light chain mRNA in a B cell to thereby suppress antibody production by the B cell. Accordingly, in one embodiment, the nucleic acid associated with/encapsulated by the lipid-based compositions, including LNPs, of the disclosure is an anti-antibody nucleic acid that functions to suppress antibody production in B cells.

Another approach for direct suppression of antibody production by B cells is through activation of one or more regulatory pathways in the B cells that results in inhibition of antibody production by the B cells. For example, signaling through the inhibitory FcgRIIB receptor on B cells independent of antigen (e.g., though overexpression of the receptor or ligation of the receptor independent of antigen) has been shown to lead to inhibition of antibody production in the B cells (see e.g., Yang et al. (2009) Blood 114:3736; Tzeng et al. (2015) J. Biomed. Sci. 22:87). Accordingly, in one embodiment, the nucleic acid (e.g., mRNA) that is used for suppressing antibody production encodes the FcgRIIB receptor such that FcgRIIB is overexpressed in B cells as means to suppress antibody production by the B cell.

In another embodiment, antibody production is indirectly suppressed using a nucleic acid that modulates the activity of a T cell involved in the regulation of antibody production. In particular, follicular helper T cells (T_{FH} cells) are responsible for providing the helper function to B cells for antibody production, whereas follicular regulatory T cells (T_{FR} cells) have the ability to suppress T_{FH} cell-mediated antibody production (see e.g., Sage et al. (2016) Nat. Immunol. 17:1436-1446). Accordingly, in one embodiment, the nucleic acid associated with/encapsulated by the lipid-based compositions, including LNPs, of the disclosure is a nucleic acid (e.g., mRNA) that functions to inhibit follicular helper T cell activity such that antibody production by B cells is suppressed. In one embodiment, cytokines known in the art to inhibit T_{FH} cell activity are encoded by the nucleic acid. In another embodiment, the nucleic acid associated with/encapsulated by the lipid-based compositions, including LNPs, of the disclosure is a nucleic acid (e.g., mRNA) that functions to stimulate follicular regulatory T cell activity such that antibody production by B cells is suppressed. For example, in one aspect, cytokines known in the art to stimulate T_{FR} cell activity are encoded by the nucleic acid.

Modulation of B_{Reg} Differentiation

B_{Reg} cells are a subpopulation of B cells that function to down modulate immune responses. The suppressive functions of B_{Reg} cells are mediated by the release of cytokines (such as IL-10, IL-12, IL-35 and TGFβ) that cause downregulation of antigen presenting cell function, inhibition of T effector cell function and induction of regulatory T cells. Decreased levels of B_{Reg} cells have been reported in numerous autoimmune diseases (see e.g., Mavropoulos et al. (2016) Arthr. Rheum. 68:494-504; Mavropoulos et al. (2017) Clin. Immunol. 184:33-41; Aybar et al. (2015) Clin. Exp. Immunol. 180:178-188), suggesting that increasing the level of B_{Reg} cells may be of benefit in alleviating autoimmunity.

Accordingly, in one embodiment of the method for inhibiting immune responses, the nucleic acid associated with/encapsulated by the lipid-based compositions, including LNPs, of the disclosure is a nucleic acid that functions to regulate B_{Reg} cells, such as induce, expand and/or or maintain B_{Reg} cells, such that an immune response is inhibited (e.g., an mRNA that encodes a protein that functions to induce, expand and/or maintain B_{Reg} cells). In one embodiment, the nucleic acid that functions to induce, expand and/or maintain B_{Reg} cells is expressed in B cells such that B_{Reg} cells are induced, expanded and/or maintained. Additionally or alternatively, in another embodiment, the nucleic acid that functions to induce, expand and/or maintain B_{Reg} cells is expressed in non-B cells (e.g., dendritic, myeloid and/or T cells) such that B_{Reg} cells are induced, expanded and/or maintained. In one embodiment, the nucleic acid that functions to induce, expand and/or maintain B_{Reg} cells is a B_{Reg}-inducing cytokine. In another embodiment, the nucleic acid that functions to induce, expand and/or maintain B_{Reg} cells modulates a signaling pathway involved in B_{Reg} development.

Numerous cytokines have been described in the art that have been demonstrated to be involved in inducing, expanding and/or maintaining B_{Reg} cells. For example, IL-6 and IL-1β have been shown to directly promote B_{Reg} cell differentiation (see e.g., Rosser et al. (2014) Nature Med. 20:1334-1339). Thus, in one embodiment, a nucleic acid that encodes IL-6 and/or IL-1β (e.g., mRNA encoding IL-6 and/or IL-1β) is expressed in immune cells to thereby induce, expand and/or maintain B_{Reg} cells.

Furthermore, IL-21 has been demonstrated to promote the development of B_{Reg} cells (see e.g., Yoshizaki et al. (2012) Nature 491:264-268). Accordingly, in one embodiment, a nucleic acid (e.g., mRNA) that encodes IL-21 is expressed in immune cells to thereby induce, expand and/or maintain B_{Reg} cells.

Additionally, GM-CSF and IL-15 have been implicated in B_{Reg} cell differentiation (see e.g., Rafei et al. (2009) Nature Med. 15:1038-1045). Thus, in one embodiment, a nucleic acid that encodes GM-CSF and/or IL-15 (e.g., mRNA encoding GM-CSF and/or IL-15) is expressed in immune cells to thereby induce, expand and/or maintain B_{Reg} cells.

Still further, IL-35 has been shown to induce B_{Reg} cells and promote their conversion to a B_{Reg} cell subset that produces IL-35 as well as IL-10 (see e.g., Wang et al. (2014) Nature Med. 20:633-641). Accordingly, in one embodiment, a nucleic acid (e.g., mRNA) that encodes IL-35 is expressed in immune cells to thereby induce, expand and/or maintain B_{Reg} cells.

In addition to B_{Reg} cell-promoting cytokines, activation of certain signaling pathways in B cells has been linked to the development of B_{Reg} cells. For example, CD40 stimulation (i.e., activation of the CD40-mediated signaling pathway) has been shown to be involved in B_{Reg} cell induction (see e.g., Yoshizaki et al. (2012) Nature 491:264-268). Accordingly, in one embodiment, a nucleic acid (e.g., mRNA) that functions to stimulate the CD40 signaling pathway is expressed in B cells to thereby induce, expand and/or maintain B_{Reg} cells.

Additionally, Toll-like receptor ligation (e.g., TLR9, TLR4, TLR2) has been demonstrated to be involved in the development of B_{Reg} cells (see e.g., Miles et al. (2012) Proc. Natl. Acad. Sci. USA 109:887-892; Meyer-Bahlburg and Rawlings (2012) Front. Biosci. 17:1499-1516; van der Vlugt et al. (2014) Meth. Mol. Biol. 1190:127-141). Accordingly, in one embodiment, a nucleic acid (e.g., mRNA) that functions to stimulate a TLR signaling pathway (e.g., a TLR9, TLR4 and/or TLR2 signaling pathway) is expressed in B cells to thereby induce, expand and/or maintain B_{Reg} cells.

T effector Cell Modulation

In one embodiment of the method for inhibiting immune responses, the nucleic acid associated with/encapsulated by the lipid-based compositions, including LNPs, of the disclosure is a nucleic acid that modulates (e.g., upregulates or downregulates) the activity of an effector T cell such that an immune response is inhibited. In some embodiments, the nucleic acid itself modulates effector T cell activity (e.g., such as siRNAs, mIRs and/or antagomirs). In some embodiments, the nucleic acid encodes a peptide or polypeptide that modulates effector T cell activity (e.g., mRNA). In some embodiments, targeting of the nucleic acid to an effector T cell directly results in the inhibition of an immune response. In some embodiments, targeting of the nucleic acid to a non-effector T cell (e.g., a dendritic cell, B cell, Treg) results in the modulation of effector T cell activity and inhibition of an immune response. For example, in some embodiments, targeting of the nucleic acid to a dendritic or myeloid cell results in the modulation of effector T cell activity.

In one embodiment, the nucleic acid encodes or modulates (e.g., upregulates or downregulates) the activity of a naturally-occurring molecule. In one embodiment, the naturally-occurring molecule is an intracellular molecule, such as a transcription factor or cell signaling cascade molecule. In another embodiment, the naturally-occurring molecule is a secreted molecule, such as a cytokine or a chemokine. In another embodiment, the naturally-occurring molecule is a transmembrane molecule, such as surface receptors, homing molecules, signaling molecules or immune modulation molecules (e.g., HLA molecules).

In one embodiment, the nucleic acid encodes a modified (i.e., non-naturally-occurring) molecule. Non-limiting examples of modified proteins include proteins having an altered (e.g., increased or decreased) half-life and proteins modified such that they are tethered to a cell surface (e.g., addition of a transmembrane domain to a soluble protein such that the modified protein tethers the soluble protein to a cell surface).

In another embodiment, the nucleic acid encodes a modified (i.e., non-naturally-occurring) transmembrane or intracellular protein. Non-limiting examples of modified transmembrane proteins include chimeric receptors, dominant-negative receptors and constitutively-active mutant receptors. Non-limiting examples of modified intracellular proteins include mutant/altered enzymes (e.g., kinases, phosphatases, dioxygenases and the like) that participate in signaling cascades, including molecules that can alter (e.g., invert) the signaling at an immune synapse (e.g., agonize or antagonize signaling through T cell receptors (TCR) or B cell receptors (BCR)), as well as molecules that can modulate (e.g., induce) apoptosis.

Non-limiting examples of specific approaches for modulating effector T cell activity to thereby inhibit immune responses are described in further detail below.

### Induction of Effector T Cell Anergy

In one embodiment of the method for inhibiting immune responses, the nucleic acid associated with/encapsulated by the lipid-based compositions, including LNPs, of the disclosure is a nucleic acid that stimulates (e.g., promotes, enhances, upregulates) the induction of effector T cell anergy such that an immune response is inhibited (e.g., a nucleic acid that encodes a protein that stimulates the induction of effector T cell anergy). In the thymus, T cells with high affinity T cell receptors that recognize self-antigens are deleted through negative selection. Self-reactive T cells can escape negative selection and may cause autoimmune reactions in the periphery, leading to autoimmune disease. T cell anergy is a mechanism of peripheral tolerance that controls the activation of self-reactive mature T cells by the induction of a long-term state of hyporesponsiveness that is established in response to suboptimal stimulation. T cells receive signals that result not only from antigen recognition and co-stimulation but also from other sources including cytokine receptors, inhibitory receptors or metabolic sensors. Integration of all those signals determines T cell fate. Under conditions that induce anergy, T cells activate a program of gene expression that leads to the production of proteins that block T cell receptor signaling and inhibit cytokine gene expression.

### Mechanisms of T Cell Anergy

The primary determinant of T cell fate (activate or become anergic) is the presence or strength of co-stimulation provided by the binding of CD28 to its ligands, CD80 and CD86 expressed on antigen presenting cells (Schwartz (2003) Annu Rev Immunol 21:305-334; Harding et al., (1992) Nature 356:607-609; Jenkins et al., (1987) J Exp Med 165:302-319; Quill et al., (1987) J Immunol 138:3704-3712; Macian et al., (2004) Curr Opin Immunol 16:209-216).

Accordingly, in one embodiment, the nucleic acid that induces effector T cell anergy inhibits CD28 co-stimulation, directly or indirectly. In another embodiment, the nucleic acid encodes a protein that inhibits CD28 co-stimulation. CD28 and CTLA-4 are highly homologous and compete for the same ligands (CD80 and CD86) (Linsley et al., (1990) Proc Natl Acad Sci USA 87(13):5031-5). CTLA4 binds these ligands with a higher affinity than CD28, which allows CTLA4 to compete with CD28 for ligand and suppress effector T cells responses (Engelhardt et al., (2006) J Immunol 177(2):1052-61). In one embodiment, the nucleic acid that inhibits CD28 co-stimulation encodes CTLA-4. In another embodiment, the nucleic acid that inhibits CD28 co-stimulation encodes a CD28-binding protein that inhibits ligand binding. In another embodiment, the nucleic acid that inhibits CD28 co-stimulation encodes a CD80/CD86-binding protein that inhibits CD28 binding. In some embodiments the CD80/CD86-binding protein is CTLA4-Ig.

CD28 engagement with ligand induces the expression and increased stabilization of 112 mRNA (Lindstein et al., (1989) Science 244:339-343). Production of IL-2 promotes anergy avoidance and signaling through the IL-2 receptor has been shown to prevent the establishment of anergy in the absence of CD28 co-stimulation (Boussiotis et al., (1994) Science 266:1039-1042). Accordingly, in some embodiments, the nucleic acid that induces effector T cell anergy decreases the production of IL-2. In some embodiments, the nucleic acid that decreases production of IL-2 inhibits transcription of IL2 mRNA. In some embodiments, the nucleic acid that decreases production of IL-2 inhibits translation of 112 mRNA.

### Inhibition of Effector T Cell Migration

In one embodiment of the method for inhibiting immune responses, the nucleic acid associated with/encapsulated by the lipid-based compositions, including LNPs, of the disclosure is a nucleic acid that inhibits (e.g., decreases, downregulates) the effector T cell migration such that an immune response is inhibited (e.g., a nucleic acid that encodes a protein that inhibits effector T cell migration). Immune surveillance and the development of effective adaptive immune responses require precise regulation of spatial and temporal lymphocyte trafficking. Dysregulation of lymphocyte activation and migration can lead to impaired adaptive immunity and chronic inflammation. In some autoimmune diseases (e.g., rheumatoid arthritis) pathological inflammation is partially dependent on migration of inflammatory cells and their retention at the inflammation site (Pope (2002) Nat Rev Immunol 2(7):527-35). T cell trafficking to the sites of inflammation is enabled by local activation in synovial vessels of the mechanisms necessary for leukocyte recruitment; alterations in these mechanisms can lead to chronic inflammation and autoimmunity. In response to proinflammatory mediators, leukocytes and vascular cells are activated. Among other immune cells, T cells (Th1, Th17, Treg, and possibly Th22) initiate a serial cascade (rolling, arrest, spreading, crawling, and transmigration) and eventually extravasate from blood vessels to the site of inflammation. Numerous cytokines, selectins, integrins, adhesion molecules, chemokines, and chemokine receptors are involved in T cell recruitment and retention. Accordingly in some embodiments, the nucleic acid that inhibits effector T cell migration inhibits the expression of a protein selected from the group consisting of: a cytokine, a selectin, an integrin, an integrin, an adhesion molecule, a chemokine and a chemokine receptor.

Leukocyte adhesion requires the coordinated action of rolling, adhesion, and transmigration events. Leukocyte subsets that express and display the appropriate adhesion molecules and chemoattractants are recruited to specific sites. Leukocyte rolling is mediated by selectins, which are expressed by most leukocyte populations (L-selectin) and by inflamed endothelial cells (E- and P-selectins) (Patel et a., (2002) Semin Immunol. 14(2):73-81). Rolling involves selectins and P-selectin glycoprotein ligand-1 (PSGL-1) which are expressed by leukocytes and inflamed endothelial cells. Interaction between PSLG1 and L-selectin is required for leukocyte-leukocyte interactions that enable leukocyte tethering and adhesion to the inflamed endothelium in conditions of blood flow (Zarbock et al, (2011) Blood 118(26):6743-6751).

Accordingly in some embodiments, the nucleic acid that inhibits effector T cell migration inhibits the expression of a selectin. In some embodiments, the selectin is a L-, E- or P-selectin. In some embodiments the selectin is P-selectin glycoprotein ligand-1 (PSGL-1).

Integrins are involved in rolling and firm leukocyte adhesion and arrest (Nourshargh et al., Immunity (2014) 41(5):694-707). Distinct cell types express specific integrins. For example, α1 integrins are strongly expressed in activated CD4+ and CD8+ T cells. Th17 cells upregulate α2 integrins. Antagonists of integrins and their ligands have been shown to prevent inflammation in the murine collagen-induced arthritis model (de Fougerolles et al., (2000) J Clin Invest 105(6):721-9). Transendothelial migration following chemotactic gradients is the final step in leukocyte migration through paracellular or transcellular pathways into inflamed tissues. The specificity of this process is achieved through differential expression of the distinct components of this leukocyte adhesion cascade, including selectins, integrins, chemokines, and their respective ligands or receptors. For example, naïve T cells express low LFA-1, α4 integrin, and CCR7 levels, which allow cell recirculation through lymphoid tissues but it is insufficient to permit cells entry into inflamed tissues. In contrast, T effector and memory cells with elevated expression of LFA-1, α-integrins, E- and P-selectin ligands, CCR1, CCR5, and CXCR3 enter these tissues.

Accordingly in some embodiments, the nucleic acid that inhibits effector T cell migration inhibits the expression of an integrin.

### Chemokines

Due to their central role in the selective recruitment and activation of immune cells at the inflammation site, chemokines and chemokine receptors are currently considered to potential therapeutic targets in several chronic autoimmune disorders. For examples, Th17 cells contribute to initiation and inflammatory phases some autoimmune disorders (e.g. RA). Although Th17 cells express other chemokine receptors such as CCR4, CCR10, and CXCR3 (126, 127), they are characterized by CCR6 expression (Lim et al., (2008) J Immunol 180(1):122-9). CCL20, the CCR6 ligand, is a selective chemoattractant for T cells, naïve B cells, and immature DCs. CCR6+ Th17 cells have been identified in peripheral blood, synovial fluid, and inflamed tissue. Expression of other chemokine receptors in CCR6+ Th cells is associated with the expression of specific sets of cytokines. CCR4+/CCR6+ Th cells express high IL-17A levels, whereas levels of this interleukin are low in CXCR3+/CCR6+ cells, whose IFN-γ levels are high. CCR6+/CCR10+ Th cells express high levels of IL-22, which defines the Th22 cell population. Other chemokine receptors found in CCR6+ Th cells are CCR5, CXCR4, and CXCR6, although they have not been associated with specific cytokine profiles. This cytokine production attracts and activates other cell types to the site of inflammation, including monocytes, neutrophils, synovial, and osteoclasts, which contribute to disease progression (Paulissen et al., (2015) Cytokine 74(1):43-53).

Accordingly, in some embodiments, the nucleic acid that inhibits effector T cell migration inhibits the expression of a chemokine or chemokine receptor in an immune cell.

### Effector T Cell Depletion

In one embodiment of the method for inhibiting immune responses, the nucleic acid associated with/encapsulated by the lipid-based compositions, including LNPs, of the disclosure is a nucleic acid that induces (e.g., promotes, increases) effector T cell depletion such that an immune response is inhibited.

It is known that activated T cells die by apoptosis by two different routes: 1) activation-induced cell death (AICD) and 2) disappearance of survival signals. When an activated T cell receives a TCR-signal, it up-regulates FasL and kills itself or its neighbor via a Fas-FasL interaction Alderson et al., (1995) J Exp Med 1995; 181:71-77; Brunner et al., (1995) Nature 1995; 373:441-444; Dhein et al., (1995) Nature 373:438-441; Ju et al., (1995) Nature 1995; 373:444-448.

Accordingly, in some embodiments, the nucleic acid induces effector T cell depletion by inducing T cell apoptosis. In some embodiments, the nucleic acid induces T cell apoptosis by upregulation of Fas or FasL. In some embodiments, the nucleic acid induces T cell apoptosis by the downregulation of a survival signal.

### Up Modulation of Lymphoid Cell Activity

In one embodiment, the immune cell delivery potentiating lipid compositions of the disclosure are used to stimulate (upregulate, enhance) the activation or activity of an immune cell, such as a lymphoid cell (e.g, T cell and/or B cell), for example in situations where stimulation of an immune response is desirable, such as in cancer therapy or treatment of an infectious disease (e.g., a viral, bacterial, fungal, protozoal or parasitic infection).

In one embodiment of stimulating the activation or activity of an immune cell, such as a lymphoid cell (e.g, T cell and/or B cell), the agent (e.g., mRNA) associated with/encapsulated by the lipid nanoparticle encodes a protein that is a cytokine, non-limiting examples of which include the cytokines described in the soluble targets section herein.

In one embodiment of stimulating the activation or activity of an immune cell, such as a lymphoid cell (e.g, T cell and/or B cell), the agent (e.g., mRNA) associated with/encapsulated by the lipid nanoparticle encodes a protein that is a chemokine or a chemokine receptor, non-limiting examples of which are described in the soluble targets section herein.

In one embodiment of stimulating the activation or activity of an immune cell, such as a lymphoid cell (e.g, T cell and/or B cell), the agent (e.g., mRNA) associated with/encapsulated by the lipid nanoparticle encodes a protein that is a costimulatory factor that upregulates immune responses or is an antagonist of a costimulatory factor that downregulates immune responses, non-limiting examples of which are described herein.

In one embodiment, of stimulating the activation or activity of an immune cell, such as a lymphoid cell (e.g, T cell and/or B cell), the agent (e.g., mRNA) associated with/encapsulated by the lipid nanoparticle encodes an antigen receptor, such as a T cell receptor or B cell receptor, e.g., a chimeric antigen receptor (CAR). Non-limiting examples of CARs include those described in the modified membrane-bound/transmembrane target section herein.

### Pharmaceutical Compositions

Formulations comprising lipid nanoparticles of the invention may be formulated in whole or in part as pharmaceutical compositions. Pharmaceutical compositions may include one or more lipid nanoparticles. For example, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

A lipid nanoparticle of the disclosure formulated into a pharmaceutical composition can encapsulate a single nucleic acid or multiple nucleic acids. When encapsulating multiple nucleic acids, the nucleic acids can be of the same type (e.g., all mRNA) or can be of different types (e.g., mRNA and DNA). Furthermore, multiple LNPs can be formulated into the same or separate pharmaceutical compositions. For example, the same or separate pharmaceutical compositions can comprise a first LNP and a second LNP, wherein the first and second LNP encapsulate the same or different nucleic acid molecules, wherein the first and second LNP include an immune cell delivery potentiating lipid as a component. In other embodiments, the same or separate pharmaceutical compositions can comprise a first LNP and a second LNP, wherein the first and second LNP encapsulate the same or different nucleic acid molecules, wherein the first LNP includes a immune cell delivery potentiating lipid as a component and the second LNP lacks a immune cell delivery potentiating lipid.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In one embodiment, such compositions are prepared in liquid form or are lyophylized (e.g., and stored at 4° C. or below freezing). For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Other Embodiments of the Disclosure

The disclosure relates to the following embodiments. Throughout this section, the term embodiment is abbreviated as ‘E’ followed by an ordinal. For example, E1 is equivalent to Embodiment 1.

E1. A lipid nanoparticle comprising:

(i) an ionizable lipid;

(ii) an effective amount of a phytosterol;

(iii) optionally, a non-cationic helper lipid;

(iv) optionally, a PEG-lipid;

(v) optionally a structural lipid; and

(vi) a nucleic acid molecule,

wherein the effective amount of the phytosterol enhances delivery of the nucleic acid molecule to an immune cell relative to a lipid nanoparticle lacking the phytosterol.
E2. The lipid nanoparticle of E1, wherein an intracellular concentration of the nucleic acid molecule in the immune cell is enhanced.
E3. The lipid nanoparticle of E1, wherein uptake of the nucleic acid molecule by the immune cell is enhanced.
E4. The lipid nanoparticle of E1, wherein an activity of the nucleic acid molecule in the immune cell is enhanced.
E5. The lipid nanoparticle of E1, wherein expression of the nucleic acid molecule in the immune cell is enhanced.
E6. The lipid nanoparticle of any one of E1-E5, wherein the nucleic acid molecule modulates the activation or activity of the immune cell.
E7. The lipid nanoparticle of E6, wherein the nucleic acid molecule increases the activation or activity of the immune cell.
E8. The lipid nanoparticle of E6, wherein the nucleic acid molecule decreases the activation or activity of the immune cell.
E9. The lipid nanoparticle of E1, wherein an activity of a protein encoded by the nucleic acid molecule in the immune cell is enhanced.
E10. The lipid nanoparticle of E1, wherein expression of a protein encoded by the nucleic acid molecule in the immune cell is enhanced.
E11. The lipid nanoparticle of any one of E9-E10, wherein the protein modulates the activation or activity of the immune cell.
E12. The lipid nanoparticle of E11, wherein the protein increases the activation or activity of the immune cell.
E13. The lipid nanoparticle of E11, wherein the protein decreases the activation or activity of the immune cell.
E14. The lipid nanoparticle of any one of E1-E13, wherein the immune cell is selected from the group consisting of a T cell, a dendritic cell, a macrophage and a B cell.
E15. The lipid nanoparticle of E14, wherein the immune cell is a T cell.
E16. The lipid nanoparticle of E14, wherein the immune cell is a B cell.
E17. The lipid nanoparticle of any one of E1-E16, wherein delivery is enhanced in vivo.
E18. The lipid nanoparticle of any one of E1-E17, wherein the phytosterol has a purity of greater than 70%, greater than 80% or greater than 90%.
E19. The lipid nanoparticle of any one of E1-E17, wherein the phytosterol has a purity of greater than 95%.
E20. The lipid nanoparticle of any one of E1-E17, wherein the phytosterol has a purity of 97%, 98%, or 99%.
E21. The lipid nanoparticle of any one of E1-E20, wherein the phytosterol is a sitosterol, a stigmasterol or a combination thereof.
E22. The lipid nanoparticle of E21, wherein the phytosterol comprises a sitosterol or a salt or an ester thereof.
E23. The lipid nanoparticle of E21, wherein the phytosterol comprises a stigmasterol or a salt or an ester thereof.
E24. The lipid nanoparticle of any one of E1-E20, wherein the phytosterol is beta-sitosterol

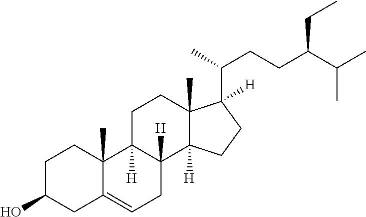

or a salt or an ester thereof.
E25. The lipid nanoparticle of E24, wherein the beta-sitosterol has a purity of greater than 70% or greater than 80%.
E26. The lipid nanoparticle of E24, wherein the beta-sitosterol has a purity of greater than 90%.
E27. The lipid nanoparticle of E24, wherein the beta-sitosterol has a purity of greater than 95%.
E28. The lipid nanoparticle of E24, wherein the beta-sitosterol has a purity of 97%, 98%, or 99%.
E29. The lipid nanoparticle of any one of E1-E28, which does not comprise a structural lipid.
E30. The lipid nanoparticle of any one of E1-E28, which comprises a structural lipid or a salt thereof.
E31. The lipid nanoparticle of E30, wherein said structural lipid is cholesterol or a salt thereof.
E32. The lipid nanoparticle of E31, wherein the mol % cholesterol is between about 1% and 50% of the mol % of phytosterol present in the lipid nanoparticle.
E33. The lipid nanoparticle of E31, wherein the mol % cholesterol is between about 10% and 40% of the mol % of phytosterol present in the lipid nanoparticle.
E34. The lipid nanoparticle of E31, wherein the mol % cholesterol is between about 20% and 30% of the mol % of phytosterol present in the lipid nanoparticle.
E35. The lipid nanoparticle of E31, wherein the mol % cholesterol is about 30% of the mol % of phytosterol present in said lipid nanoparticle.
E36. The lipid nanoparticle of any one of the preceding embodiments, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E37. The lipid nanoparticle of any one of the preceding embodiments, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E38. The lipid nanoparticle of any one of the preceding embodiments, wherein the ionizable lipid is

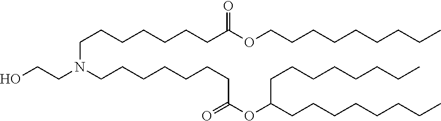 [SMILES: CCCCCCCCCOC(=O)CCCCCCCN(CCO)CCCCCCCC(=O)OC(CCCCCCCC)CCCCCCCC]

or a salt thereof.
E39. The lipid nanoparticle of any one of the preceding embodiments, wherein the ionizable lipid is

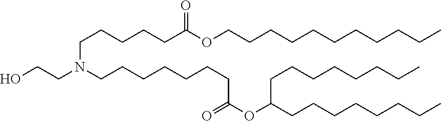 [SMILES: CCCCCCCCCCCOC(=O)CCCCCN(CCO)CCCCCCCC(=O)OC(CCCCCCCC)CCCCCCCC]

or a salt thereof.
E40. The lipid nanoparticle of any one of the preceding embodiments, which comprises a non-cationic helper lipid.
E41. The lipid nanoparticle of E40, wherein the non-cationic helper lipid is a phospholipid.
E42. The lipid nanoparticle of E41, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E43. The lipid nanoparticle of E42, wherein the phospholipid is DSPC.
E44. The lipid nanoparticle of E40, wherein the non-cationic helper lipid is oleic acid.
E45. The lipid nanoparticle of any one of the preceding embodiments, which comprises a PEG-lipid.
E46. The lipid nanoparticle of E45, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E47. The lipid nanoparticle of E45, wherein the PEG lipid is selected from the group consisting of PEG-c-DOMG, PEG-DMG, PEG-DLPE, PEG-DMPE, PEG-DPPC and PEG-DSPE lipid.
E48. The lipid nanoparticle of E47, wherein the PEG-lipid is PEG-DMG.
E49. The lipid nanoparticle of any one of the preceding embodiments, comprising about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E50. The lipid nanoparticle of any one of the preceding embodiments, comprising about 35 mol % to about 55 mol % ionizable lipid, about 5 mol % to about 25 mol % phospholipid, about 30 mol % to about 40 mol % sterol, and about 0 mol % to about 10 mol % PEG lipid.
E51. The lipid nanoparticle of any one of the preceding embodiments, comprising about 50 mol % ionizable lipid, about 10 mol % phospholipid, about 38.5 mol % sterol, and about 1.5 mol % PEG lipid.
E52. The lipid nanoparticle of any one of E1-E51, wherein the nucleic acid molecule is selected from the group consisting of RNA, mRNA, RNAi, dsRNA, siRNA, antisense RNA, ribozyme, CRISPR/Cas9, ssDNA and DNA.
E53. The lipid nanoparticle of E52, wherein the nucleic acid is RNA selected from the group consisting of a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.
E54. The lipid nanoparticle of E53, wherein the nucleic acid is an mRNA.
E55. The lipid nanoparticle of E54, wherein the mRNA is a modified mRNA comprising one or more modified nucleobases.
E56. The lipid nanoparticle of E54 or E55, wherein the mRNA includes one or more of a stem loop, a chain terminating nucleoside, a polyA sequence, a polyadenylation signal, and/or a 5′ cap structure.
E57. The lipid nanoparticle of any one of E54-E56, wherein the mRNA encodes a protein for expression in the immune cell.
E58. The lipid nanoparticle of E57, wherein the immune cell is selected from the group consisting of a T cell, a dendritic cell, a macrophage and a B cell.
E59. The lipid nanoparticle of E58, wherein the immune cell is a T cell.
E60. The lipid nanoparticle of E57, wherein the immune cell exhibits deregulated control of G1 progression.
E61. The lipid nanoparticle of any one of E57-E60, wherein the protein encoded by the mRNA increases activation or activity of the immune cell expressing the protein.
E62. The lipid nanoparticle of any one of E57-E60, wherein the protein encoded by the mRNA decreases activation or activity of the immune cell expressing the protein.
E63. The lipid nanoparticle of any one of E57-E60, wherein the protein encoded by the mRNA enhances an immune response to the immune cell expressing the protein.
E64. The lipid nanoparticle of any one of E57-E60, wherein the protein encoded by the mRNA enhances an immune response by the immune cell expressing the protein.
E65. The lipid nanoparticle of any one of E57-E60, wherein the protein encoded by the mRNA is selected from the group consisting of cytokines, chemokines, costimulatory factors, T cell Receptors (TcRs), chimeric antigen receptors (CARs), recruitment factors, transcription factors, effector molecules, and enzymes.
E66. The lipid nanoparticle of E65, wherein the protein is a costimulatory factor selected from the group consisting of, CD28, CD80, CD86, ICOS.
E67. The lipid nanoparticle of E65, wherein the immune cell is a T cell and the protein is a T cell receptor or chimeric antigen receptor that recognizes a cancer antigen.
E68. The lipid nanoparticle of E65, wherein the protein is a cytokine.
E69. The lipid nanoparticle of E68, wherein the cytokine is selected from the group consisting of IL-1, IL-2, IL-4, IL-5, IL-6, IL-7, IL-10, IL-12, IL-13, IL-15, Il-18, IL-21, TNFα, GM-CSF, and a combination thereof.
E70. The lipid nanoparticle of E65, wherein the protein is a transcription factor that increases or polarizes an immune response.
E71. The lipid nanoparticle of E65, wherein the protein decreases expression or activity of TGFβ.
E72. The lipid nanoparticle of E65, wherein the immune cell is a T cell and the protein is granzyme A/B or perforin.
E73. The lipid nanoparticle of E62, wherein the immune cell is a T cell and the protein is CTLA4.
E74. The lipid nanoparticle of E65, wherein the LNP comprises at least two mRNA molecules that encode a first and second protein, wherein the first and/or second protein increases activation or activity of an immune cell.
E75. The lipid nanoparticle of E58, wherein the immune cell is a B cell.
E76. A lipid nanoparticle comprising:

(i) an ionizable lipid;

(ii) an effective amount of a phytosterol;

(iii) a non-cationic helper lipid;

(iv) a PEG-lipid; and

(v) an mRNA encoding a protein of interest,

wherein the effective amount of the phytosterol enhances delivery of the mRNA to an immune cell relative to a lipid nanoparticle lacking the phytosterol.
E77. The lipid nanoparticle of E76, wherein an intracellular concentration of the mRNA in the immune cell is enhanced.
E78. The lipid nanoparticle of E76, wherein uptake of the mRNA by the immune cell is enhanced.
E79. The lipid nanoparticle of E76, wherein an activity of the mRNA in the immune cell is enhanced.
E80. The lipid nanoparticle of E76, wherein expression of the mRNA in the immune cell is enhanced.
E81. The lipid nanoparticle of E76, wherein an activity of the protein encoded by the mRNA in the immune cell is enhanced.
E82. The lipid nanoparticle of E76, wherein expression of the protein encoded by the mRNA in the immune cell is enhanced.
E83. The lipid nanoparticle of any of E81 or E82, wherein the protein modulates the activation or activity of the immune cell.
E84. The lipid nanoparticle of E83, wherein the protein increases the activation or activity of the immune cell.
E85. The lipid nanoparticle of E83, wherein the protein decreases the activation or activity of the immune cell.
E86. The lipid nanoparticle of any one of E76-E85, wherein the immune cell is selected from the group consisting of a T cell, a dendritic cell, a macrophage and a B cell.
E87. The lipid nanoparticle of E85, wherein the immune cell is a T cell.
E88. The lipid nanoparticle of E85, wherein the immune cell is a B cell.
E89. The lipid nanoparticle of any one of E76-E88, wherein delivery is enhanced in vivo.
E90. The lipid nanoparticle of any one of E76-E89, wherein the phytosterol has a purity of greater than 70%, greater than 80% or greater than 95%.
E91. The lipid nanoparticle of any one of E76-E89, wherein the phytosterol has a purity of 97%, 98%, or 99%.
E92. The lipid nanoparticle of any one of E76-E91, wherein the phytosterol is a sitosterol, a stigmasterol or a combination thereof.
E93. The lipid nanoparticle of E92, wherein the phytosterol comprises a sitosterol or salt or ester thereof.
E94. The lipid nanoparticle of E92, wherein the phytosterol comprises a stigmasterol or salt or ester thereof.
E95. The lipid nanoparticle of any one of E76-E91, wherein the phytosterol is beta-sitosterol

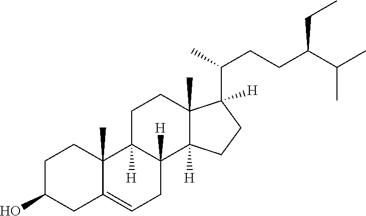

or a salt or an ester thereof.
E96. The lipid nanoparticle of any one of E75-E90, wherein the beta-sitosterol has a purity of greater than 70%, greater than 80%, or greater than 90%.
E97. The lipid nanoparticle of E95, wherein the beta-sitosterol has a purity of greater than 95%.
E98. The lipid nanoparticle of E95, wherein the beta-sitosterol has a purity of 97%, 98%, or 99%.
E99. The lipid nanoparticle of any one of E76-E98, which does not comprise a structural lipid.
E100. The lipid nanoparticle of any one of E76-E98, which further comprises a structural lipid or a salt thereof.
E101. The lipid nanoparticle of E100, wherein said structural lipid is cholesterol or a salt thereof.
E102. The lipid nanoparticle of E101, wherein the mol % cholesterol is between about 1% and 50% of the mol % of phytosterol present in the lipid nanoparticle.
E103. The lipid nanoparticle of E101, wherein the mol % cholesterol is between about 10% and 40% of the mol % of phytosterol present in the lipid nanoparticle.
E104. The lipid nanoparticle of E101, wherein the mol % cholesterol is between about 20% and 30% of the mol % of phytosterol present in the lipid nanoparticle.
E105. The lipid nanoparticle of E101, wherein the mol % cholesterol is about 30% of the mol % of phytosterol present in said lipid nanoparticle.
E106. The lipid nanoparticle of any one of E76-E105, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E107. The lipid nanoparticle of any one of E76-E106, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E108. The lipid nanoparticle of any one of E76-E107, wherein the ionizable lipid is

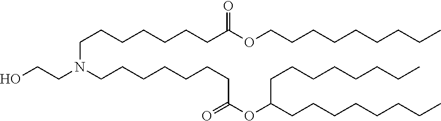 [SMILES: CCCCCCCCCOC(=O)CCCCCCCN(CCO)CCCCCCCC(=O)OC(CCCCCCCC)CCCCCCCC]

or a salt thereof.
E109. The lipid nanoparticle of any one of E76-E107, wherein the ionizable lipid is

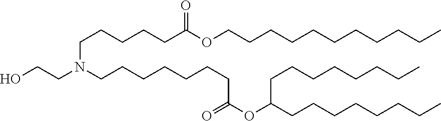 [SMILES: CCCCCCCCCCCOC(=O)CCCCCN(CCO)CCCCCCCC(=O)OC(CCCCCCCC)CCCCCCCC]

or a salt thereof.
E110. The lipid nanoparticle of any one of E76-E109, wherein the non-cationic helper lipid is a phospholipid.
E111. The lipid nanoparticle of E110, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E112. The lipid nanoparticle of E111, wherein the phospholipid is DSPC.
E113. The lipid nanoparticle of any one of E76-E109, wherein the non-cationic helper lipid is oleic acid.
E114. The lipid nanoparticle of any one of E76-E113, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E115. The lipid nanoparticle of E114, wherein the PEG lipid is selected from the group consisting of PEG-c-DOMG, PEG-DMG, PEG-DLPE, PEG-DMPE, PEG-DPPC and PEG-DSPE lipid.
E116. The lipid nanoparticle of E115, wherein the PEG lipid is PEG-DMG.
E117. The lipid nanoparticle of any one of E76-E116, comprising about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E118. The lipid nanoparticle of any one of E76-E117, comprising about 35 mol % to about 55 mol % ionizable lipid, about 5 mol % to about 25 mol % phospholipid, about 30 mol % to about 40 mol % sterol, and about 0 mol % to about 10 mol % PEG lipid.
E119. The lipid nanoparticle of any one of E76-E118, comprising about 50 mol % ionizable lipid, about 10 mol % phospholipid, about 38.5 mol % sterol, and about 1.5 mol % PEG lipid.
E120. The lipid nanoparticle of any one of E76-E119, wherein the mRNA is a modified mRNA comprising one or more modified nucleobases.
E121. The lipid nanoparticle of any one of E76-E120, wherein the mRNA includes one or more of a stem loop, a chain terminating nucleoside, a polyA sequence, a polyadenylation signal, and/or a 5′ cap structure.
E122. The lipid nanoparticle of any one of E76-E121, wherein immune cell exhibits deregulated control of G1 progression.
E123. The lipid nanoparticle of any one of E76-E122, wherein the protein encoded by the mRNA increases activation or activity of the immune cell expressing the protein.
E124. The lipid nanoparticle of any one of E76-E122, wherein the protein encoded by the mRNA decreases activation or activity of the immune cell expressing the protein.
E125. The lipid nanoparticle of any one of E76-E122, wherein the protein encoded by the mRNA enhances an immune response to the immune cell expressing the protein.
E126. The lipid nanoparticle of any one of E76-E122, wherein the protein encoded by the mRNA enhances an immune response by the immune cell expressing the protein.
E127. The lipid nanoparticle of any one of E76-E122, wherein the protein encoded by the mRNA is selected from the group consisting of cytokines, chemokines, costimulatory factors, T cell Receptors (TcRs), chimeric antigen receptors (CARs), recruitment factors, transcription factors, effector molecules, and enzymes.
E128. The lipid nanoparticle of E127, wherein the protein is a costimulatory factor selected from the group consisting of, CD28, CD80, CD86, ICOS.
E129. The lipid nanoparticle of E127, wherein the immune cell is a T cell and the protein is a T cell receptor or chimeric antigen receptor that recognizes a cancer antigen.
E130. The lipid nanoparticle of E127, wherein the protein is a cytokine.
E131. The lipid nanoparticle of E130, wherein the cytokine is selected from the group consisting of IL-1, IL-2, IL-4, IL-5, IL-6, IL-7, IL-10, IL-12, IL-13, IL-15, Il-18, IL-21, TNFα, GM-CSF, and a combination thereof.
E132. The lipid nanoparticle of E127, wherein the protein is a transcription factor that increases or polarizes an immune response.
E133. The lipid nanoparticle of E127, wherein the protein decreases expression or activity of TGFβ.
E134. The lipid nanoparticle of E127, wherein the immune cell is a T cell and the protein is granzyme A/B or perforin.
E135. The lipid nanoparticle of E124, wherein the immune cell is a T cell and the protein is CTLA4.
E136. The lipid nanoparticle of E127, wherein the LNP comprises at least two mRNA molecules that encode a first and second protein, wherein the first and/or second protein increases activation or activity of the immune.
E137. A method of delivering a nucleic acid molecule to an immune cell, the method comprising contacting the immune cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) a nucleic acid molecule,

such that the nucleic acid molecule is delivered to the immune cell.
E138. The method of E137, wherein the nucleic acid molecule is delivered to the immune cell in vivo.
E139. The method of E137, wherein an intracellular concentration of the nucleic acid molecule in the immune cell is enhanced.
E140. The method of E137, wherein uptake of the nucleic acid molecule by the immune cell is enhanced.
E141. The method of E137, wherein an activity of the nucleic acid molecule in the immune cell is enhanced or expression of the nucleic acid molecule in the immune cell is enhanced.
E142. The method of any one of E137-E141, wherein the nucleic acid molecule modulates the activation or activity of the immune cell.
E143. The method of E142, wherein the nucleic acid molecule increases the activation or activity of the immune cell.
E144. The method of E142, wherein the nucleic acid molecule decreases the activation or activity of the immune cell.
E145. The method of E137, wherein an activity of a protein encoded by the nucleic acid molecule in the immune cell is enhanced.
E146. The method of E137, wherein expression of a protein encoded by the nucleic acid molecule in the immune cell is enhanced.
E147. The method of any one of E145 or E146, wherein the protein modulates the activation or activity of the immune cell.
E148. The method of E147, wherein the protein increases the activation or activity of the immune cell.
E149. The method of E147, wherein the protein decreases the activation or activity of the immune cell.
E150. The method of any one of E137-E149, wherein the immune cell is a T cell.
E151. The method of any one of E137-E149, wherein the immune cell is a B cell.
E152. The method of any one of E137-E149, wherein the immune cell is selected from the group consisting of B cells, macrophages and dendritic cells.
E153. The method of any one of E137-E152, which further comprises administering, concurrently or consecutively, a second LNP encapsulating the same or different nucleic acid molecule, wherein the second LNP lacks a phytosterol.
E154. The method of any one of E137-E152, which further comprises administering, concurrently or consecutively, a second LNP encapsulating a different nucleic acid molecule, wherein the second LNP comprises a phytosterol.
E155. A method of inducing expression of a protein of interest in or on an immune cell, the method comprising contacting the immune cell with a lipid nanoparticle comprising:

(i) an ionizable lipid;

(ii) a phytosterol;

(iii) optionally, a non-cationic helper lipid;

(iv) optionally, a PEG-lipid;

(v) optionally, a structural lipid; and

(vi) a nucleic acid molecule encoding the protein of interest,

such that expression of the protein of interest is induced in or on the immune cell.
E156. The method of E155, wherein the protein encoded by the nucleic acid increases activation or activity of the immune cell expressing the protein.
E157. The method of E155, wherein the protein encoded by the nucleic acid decreases activation or activity of the immune cell expressing the protein.
E158. The method of E155, wherein the protein encoded by the nucleic acid enhances an immune response to the immune cell expressing the protein.
E159. The method of E155, wherein the protein encoded by the nucleic acid enhances an immune response by the immune cell expressing the protein.
E160. The method of E155, wherein the protein encoded by the nucleic acid is selected from the group consisting of cytokines, chemokines, costimulatory factors, T cell Receptors (TcRs), chimeric antigen receptors (CARs), recruitment factors, transcription factors, effector molecules.
E161. The method of any one of E155-E160, wherein the immune cell is a T cell.
E162. The method of any one of E155-E160, wherein the immune cell is a B cell.
E163. The method of any one of E155-E162, which further comprises administering, concurrently or consecutively, a second LNP encapsulating the same or different nucleic acid molecule, wherein the second LNP lacks a phytosterol.
E164. The method of any one of E155-E162, which further comprises administering, concurrently or consecutively, a second LNP encapsulating a different nucleic acid molecule, wherein the second LNP comprises a phytosterol.
E165. A method of modulating T cell activation or activity, the method comprising contacting a T cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) a nucleic acid molecule,

such that T cell activation or activity is modulated.
E166. The method of E165, wherein T cell activation or activity is enhanced.
E167. The method of E165, wherein T cell activation or activity is reduced.
E168. The method of any one of E165-E167, which further comprises administering, concurrently or consecutively, a second LNP encapsulating the same or different nucleic acid molecule, wherein the second LNP lacks a phytosterol.
E169. The method of any one of E165-E167, which further comprises administering, concurrently or consecutively, a second LNP encapsulating a different nucleic acid molecule, wherein the second LNP comprises a phytosterol.
E170. A method of increasing an immune response to a protein, the method comprising contacting immune cells with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) a nucleic acid molecule,

such that the immune response to the protein is increased.
E171. The method of E170, wherein the protein is an antigen.
E172. The method of E171, wherein the protein is a cancer antigen.
E173. The method of E171, wherein the protein is an infectious disease antigen.
E174. The method of E170, wherein the immune cells are T cells.
E175. The method of E170, wherein the immune cells are B cells.
E176. The method of E171, wherein the nucleic acid molecule encodes the protein to which an immune response is enhanced.
E177. The method of E171, wherein the nucleic acid molecule encodes a protein different than the protein to which an immune response is enhanced.
E178. The method of any one of E169-E177, which further comprises administering, concurrently or consecutively, a second LNP encapsulating the same or different nucleic acid molecule, wherein the second LNP lacks a phytosterol.
E179. The method of any one of E169-E177, which further comprises administering, concurrently or consecutively, a second LNP encapsulating a different nucleic acid molecule, wherein the second LNP comprises a phytosterol.
E180. A method of increasing a T cell response to a cancer antigen, the method comprising contacting the T cell with a lipid nanoparticle comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an mRNA encoding a chimeric antigen receptor (CAR) that binds the cancer antigen,

such that the T cell response to the cancer antigen is increased.
E181. The method of E180, wherein the cancer antigen is CD33 and the CAR is an anti-CD33 CAR.
E182. The method of E181, wherein the T cell response to CD33+ acute myelocytic leukemia (AML) cells is increased.
E183. The method of any one of E180-E182, which further comprises administering, concurrently or consecutively, a second LNP encapsulating the same or different mRNA, wherein the second LNP lacks a phytosterol.
E184. The method of any one of E180-E183, which further comprises administering, concurrently or consecutively, a second LNP encapsulating a different mRNA, wherein the second LNP comprises a phytosterol.
E185. The method of any one of E137-E184, wherein the immune cell or T cell is contacted with the lipid nanoparticle in vitro.
E186. The method of any one of E137-E184, wherein the immune cell or T cell is contacted with the lipid nanoparticle in vivo by administering the lipid nanoparticle to a subject.
E187. The method of E186, wherein the lipid nanoparticle is administered intravenously.
E188. The method of E186, wherein the lipid nanoparticle is administered intramuscularly.
E189. The method of E186, wherein the lipid nanoparticle is administered by a route selected from the group consisting of subcutaneously, intranodally and intratumorally.
E190. The method of any one of E155-E179, wherein an intracellular concentration of the nucleic acid molecule in the immune cell or T cell is enhanced.
E191. The method of any one of E155-E179, wherein an activity of the nucleic acid molecule in the immune cell or T cell is enhanced.
E192. The method of any one of E155-E179, wherein expression of the nucleic acid molecule in the immune cell or T cell is enhanced.
E193. The method of any one of E155-E179, wherein the nucleic acid molecule modulates the activation or activity of the immune cell or T cell.
E194. The method of any one of E155-E179, wherein the nucleic acid molecule increases the activation or activity of the immune cell or T cell.
E195. The method of any one of E155-E179, wherein the nucleic acid molecule decreases the activation or activity of the immune cell or T cell.
E196. The method of any one of E155-E164 and E170-E179, wherein the immune cell is selected from the group consisting of a T cell, a dendritic cell, a macrophage and a B cell.
E197. The method of E196, wherein the immune cell is a T cell.
E198. The method of E196, wherein the immune cell is a B cell.
E199. The method of any one of E137-E198, wherein the phytosterol has a purity of greater than 70%, greater than 80%, greater than 90% or greater than 95%.
E200. The method of any one of E137-E198, wherein the phytosterol has a purity of 97%, 98%, or 99%.
E201. The method of any one of E137-E200, wherein the phytosterol is a sitosterol, a stigmasterol or a combination thereof.
E202. The method of E201, wherein the phytosterol comprises a sitosterol or salt or ester thereof.
E203. The method of E201, wherein the phytosterol comprises a stigmasterol or salt or ester thereof.
E204. The method of any one of E137-E200, wherein the phytosterol is beta-sitosterol

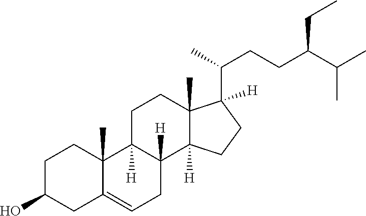

or a salt or an ester thereof.
E205. The method of E203, wherein the beta-sitosterol has a purity of greater than 70% or greater than 80% or greater than 90%.
E206. The method of E204, wherein the beta-sitosterol has a purity of greater than 95%.
E207. The method of E204, wherein the beta-sitosterol has a purity of 97%, 98%, or 99%.
E208. The method of any one of E137-E207, which does not comprise a structural lipid.
E209. The method of any one of E137-E208, wherein the lipid nanoparticle comprises a structural lipid or a salt thereof.
E210. The method of E209, wherein said structural lipid is cholesterol or a salt thereof.
E211. The method of E210, wherein the mol % cholesterol is between about 1% and 50% of the mol % of phytosterol present in the lipid nanoparticle.
E212. The method of E210, wherein the mol % cholesterol is between about 10% and 40% of the mol % of phytosterol present in the lipid nanoparticle.
E213. The method of E210, wherein the mol % cholesterol is between about 20% and 30% of the mol % of phytosterol present in the lipid nanoparticle.
E214. The method of E210, wherein the mol % cholesterol is about 30% of the mol % of phytosterol present in said lipid nanoparticle.
E215. The method of any one of E137-E214, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E216. The method of any one of E137-E214, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E217. The method of any one of E137-E214, wherein the ionizable lipid is

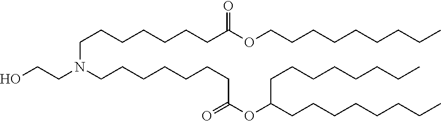 [SMILES: CCCCCCCCCOC(=O)CCCCCCCN(CCO)CCCCCCCC(=O)OC(CCCCCCCC)CCCCCCCC]

or a salt thereof.
E218. The method of any one of E137-E214, wherein the ionizable lipid is

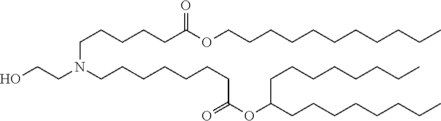 [SMILES: CCCCCCCCCCCOC(=O)CCCCCN(CCO)CCCCCCCC(=O)OC(CCCCCCCC)CCCCCCCC]

or a salt thereof.
E219. The method of any one of E137-E214, wherein the lipid nanoparticle comprises a non-cationic helper lipid.
E220. The method of E219, wherein the non-cationic helper lipid is a phospholipid.
E221. The method of E220, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E222. The method of E221, wherein the phospholipid is DSPC.
E223. The method of E219, wherein the non-cationic helper lipid is oleic acid.
E224. The method of any one of E137-E223, wherein the lipid nanoparticle comprises a PEG-lipid.
E225. The method of claim E224, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E226. The method of E224, wherein the PEG lipid is selected from the group consisting of PEG-c-DOMG, PEG-DMG, PEG-DLPE, PEG-DMPE, PEG-DPPC and PEG-DSPE lipid.
E227. The method of E226, wherein the PEG lipid is PEG-DMG.
E228. The method of any one of E137-E227, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E229. The method of any one of E137-E227, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E230. The method of any one of E137-E227, wherein the lipid nanoparticle comprises about 50 mol % ionizable lipid, about 10 mol % phospholipid, about 38.5 mol % sterol, and about 1.5 mol % PEG lipid.
E231. The method of any one of E137-180 and 185-230, wherein the nucleic acid molecule is selected from the group consisting of RNA, mRNA, RNAi, dsRNA, siRNA, antisense RNA, ribozyme, CRISPR/Cas9, ssDNA and DNA.
E232. The method of E231, wherein the nucleic acid is RNA selected from the group consisting of a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.
E233. The method of E232, wherein the nucleic acid is an mRNA.
E234. The method of E233, wherein the mRNA is a modified mRNA comprising one or more modified nucleobases.
E235. The method of E233 or E234, wherein the mRNA includes one or more of a stem loop, a chain terminating nucleoside, a polyA sequence, a polyadenylation signal, and/or a 5′ cap structure.
E236. The method of any one of E233-E235, wherein the mRNA encodes a protein for expression in the immune cell.
E237 The method of E236, wherein the immune cell is selected from the group consisting of a T cell, a dendritic cell, a macrophage and a B cell.
E238. The method of E237, wherein the immune cell is a T cell.
E239. The method of E237, wherein the immune cell is a B cell.
E240. The method of E236, wherein the immune cell exhibits deregulated control of G1 progression.
E241. A method of enhancing an immune response to an antigen of interest in a subject, the method comprising administering to the subject a lipid nanoparticle (LNP)comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an mRNA encoding the antigen of interest,

wherein the LNP comprises a phytosterol such that the immune response to the antigen of interest is enhanced in the subject, as compared to the immune response to the antigen of interest induced by an LNP encapsulating the mRNA encoding the antigen of interest but lacking the phytosterol.
E242. The method of E241, wherein the antigen of interest is a cancer antigen. 243. The method of E241, wherein the antigen of interest is an infectious disease antigen.
E244. The method of E243, wherein the antigen of interest is a bacterial antigen, a viral antigen, a fungal antigen, a protozoa antigen or a parasite antigen.
E245. The method of E241, wherein the lipid nanoparticle is administered intramuscularly.
E246. The method of E241, wherein the lipid nanoparticle is administered intradermally.
E247. The method of E241, wherein the lipid nanoparticle is administered intranodally.
E248. The method of E241, wherein the immune response is an antigen-specific antibody response.
E249. The method of E241, wherein the immune response is an antigen-specific T cell response.
E250. The method of any one of E241-E249, which further comprises administering, concurrently or consecutively, a second LNP encapsulating the same or different mRNA, wherein the second LNP lacks a phytosterol.
E251. The method of any one of E241-E249, which further comprises administering, concurrently or consecutively, a second LNP encapsulating a different mRNA, wherein the second LNP comprises a phytosterol.
E252. The method of any one of E241-E251, wherein the phytosterol has a purity of greater than 70%.
E253. The method of any one of E241-E251, wherein the phytosterol has a purity of greater than 80%.
E254. The method of any one of E241-E251, wherein the phytosterol has a purity of greater than 90%.
E255. The method of any one of E241-E251, wherein the phytosterol has a purity of greater than 95%.
E256. The method of any one of E241-E251, wherein the phytosterol has a purity of 97%, 98%, or 99%.
E257. The method of any one of E241-E256, wherein the phytosterol is a sitosterol, a stigmasterol or a combination thereof.
E258. The method of E257, wherein the phytosterol comprises a sitosterol or salt or ester thereof.
E259. The method of E257, wherein the phytosterol comprises a stigmasterol or salt or ester thereof.
E260. The method of any one of E241-E256, wherein the phytosterol is beta-sitosterol

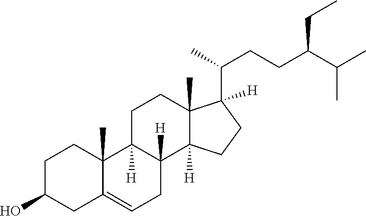

or a salt or an ester thereof.
E261. The method of E260, wherein the beta-sitosterol has a purity of greater than 70% or greater than 80%.
E262. The method of E260, wherein the beta-sitosterol has a purity of greater than 90%.
E263. The method of E260, wherein the beta-sitosterol has a purity of greater than 95%.
E264. The method of E260, wherein the beta-sitosterol has a purity of 97%, 98%, or 99%.
E265. The method of any one of E241-E264, which does not comprise a structural lipid.
E266. The method of any one of E241-E264, wherein the lipid nanoparticle comprises a structural lipid or salt thereof.
E267. The method of E266, wherein said structural lipid is cholesterol or a salt thereof.
E268. The method of E267, wherein the mol % cholesterol is between about 1% and 50% of the mol % of phytosterol present in the lipid nanoparticle.
E269. The method of E267, wherein the mol % cholesterol is between about 10% and 40% of the mol % of phytosterol present in the lipid nanoparticle.
E270. The method of E267, wherein the mol % cholesterol is between about 20% and 30% of the mol % of phytosterol present in the lipid nanoparticle.
E271. The method of E267, wherein the mol % cholesterol is about 30% of the mol % of phytosterol present in said lipid nanoparticle.
E272. The method of any one of E241-E271, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E273. The method of any one of E241-E271, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E274. The method of any one of E241-E271, wherein the ionizable lipid is

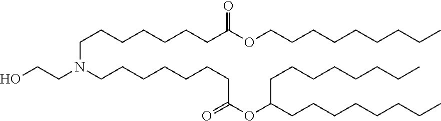 [SMILES: CCCCCCCCCOC(=O)CCCCCCCN(CCO)CCCCCCCC(=O)OC(CCCCCCCC)CCCCCCCC]

or a salt thereof.
E275. The method of any one of E241-E271, wherein the ionizable lipid is

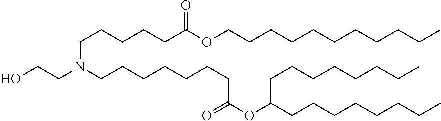 [SMILES: CCCCCCCCCCCOC(=O)CCCCCN(CCO)CCCCCCCC(=O)OC(CCCCCCCC)CCCCCCCC]

or a salt thereof.
E276. The method of any one of E241-E275, wherein the lipid nanoparticle comprises a non-cationic helper lipid.
E277. The method of E276, wherein the non-cationic helper lipid is a phospholipid.
E278. The method of E277, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E279. The method of E278, wherein the phospholipid is DSPC.
E280. The method of E276, wherein the non-cationic helper lipid is oleic acid.
E281. The method of any one of E241-E280, wherein the lipid nanoparticle comprises a PEG-lipid.
E282. The method of E281, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E283. The method of 281, wherein the PEG lipid is selected from the group consisting of PEG-c-DOMG, PEG-DMG, PEG-DLPE, PEG-DMPE, PEG-DPPC and PEG-DSPE lipid.
E284. The method of E283, wherein the PEG lipid is PEG-DMG.
E285. The method of any one of E241-E284, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E286. The method of any one of E241-E284, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E287. The method of any one of E241-E284, wherein the lipid nanoparticle comprises about 50 mol % ionizable lipid, about 10 mol % phospholipid, about 38.5 mol % sterol, and about 1.5 mol % PEG lipid.
E288. The method of any one of E241-E287, wherein the mRNA is a modified mRNA comprising one or more modified nucleobases.
E289. The method of any one of E241-E288, wherein the mRNA includes one or more of a stem loop, a chain terminating nucleoside, a polyA sequence, a polyadenylation signal, and/or a 5′ cap structure.
E290. A method of modulating B cell activation or activity, the method comprising contacting a B cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) a nucleic acid molecule,

such that B cell activation or activity is modulated.
E291. The method of E290, wherein B cell activation or activity is enhanced.
E292. The method of E290, wherein B cell activation or activity is reduced.
E293. The method of any one of E290-E292, which further comprises administering, concurrently or consecutively, a second LNP encapsulating the same or different nucleic acid molecule, wherein the second LNP lacks a phytosterol.
E294. The method of any one of E290-E292, which further comprises administering, concurrently or consecutively, a second LNP encapsulating a different nucleic acid molecule, wherein the second LNP comprises a phytosterol.
E295. The method of any one of E290-E294, wherein the B cell is contacted with the lipid nanoparticle in vitro.
E296. The method of any one of E290-E294, wherein the B cell is contacted with the lipid nanoparticle in vivo by administering the lipid nanoparticle to a subject.
E297. The method of E296, wherein the lipid nanoparticle is administered intravenously.
E298. The method of E296, wherein the lipid nanoparticle is administered intramuscularly.
E299. The method of E296, wherein the lipid nanoparticle is administered by a route selected from the group consisting of subcutaneously, intranodally and intratumorally.
E300. The method of any one of E290-E299, wherein an intracellular concentration of the nucleic acid molecule in the B cell is enhanced.
E301. The method of any one of E290-E299, wherein an activity of the nucleic acid molecule in the B cell is enhanced.
E302. The method of any one of E290-E299, wherein expression of the nucleic acid molecule in the B cell is enhanced.
E303. The method of any one of E290-E299, wherein the nucleic acid molecule modulates the activation or activity of the B cell.
E304. The method of any one of E290-E299, wherein the nucleic acid molecule increases the activation or activity of the B cell.
E305. The method of any one of E290-E299, wherein the nucleic acid molecule decreases the activation or activity of the B cell.
E306. The method of any one of E290-E305, wherein the phytosterol has a purity of greater than 70%, greater than 80%, greater than 90% or greater than 95%.
E307. The method of any one of E290-E305, wherein the phytosterol has a purity of 97%, 98%, or 99%.
E308. The method of any one of E290-E307, wherein the phytosterol is a sitosterol, a stigmasterol or a combination thereof.
E309. The method of E308, wherein the phytosterol comprises a sitosterol or salt or ester thereof.
E310. The method of E308, wherein the phytosterol comprises a stigmasterol or salt or ester thereof.
E311. The method of any one of E290-E307, wherein the phytosterol is beta-sitosterol

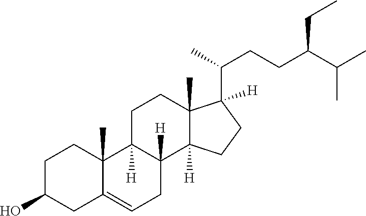

or a salt or an ester thereof.
E312. The method of E311, wherein the beta-sitosterol has a purity of greater than 70% or greater than 80% or greater than 90%.
E313. The method of E311, wherein the beta-sitosterol has a purity of greater than 95%.
E314. The method of E311, wherein the beta-sitosterol has a purity of 97%, 98%, or 99%.
E315. The method of any one of E290-E314, which does not comprise a structural lipid.
E316. The method of any one of E290-E315, wherein the lipid nanoparticle comprises a structural lipid or a salt thereof.
E317. The method of E316, wherein said structural lipid is cholesterol or a salt thereof.
E318. The method of E317, wherein the mol % cholesterol is between about 1% and 50% of the mol % of phytosterol present in the lipid nanoparticle.
E319. The method of E317, wherein the mol % cholesterol is between about 10% and 40% of the mol % of phytosterol present in the lipid nanoparticle.
E320. The method of E317, wherein the mol % cholesterol is between about 20% and 30% of the mol % of phytosterol present in the lipid nanoparticle.
E321. The method of E317, wherein the mol % cholesterol is about 30% of the mol % of phytosterol present in said lipid nanoparticle.
E322. The method of any one of E290-E321, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E323. The method of any one of E290-E321, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E324. The method of any one of E290-E321, wherein the ionizable lipid is

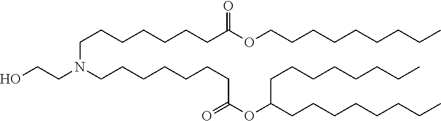 [SMILES: CCCCCCCCCOC(=O)CCCCCCCN(CCO)CCCCCCCC(=O)OC(CCCCCCCC)CCCCCCCC]

or a salt thereof.
E325. The method of any one of E290-E321, wherein the ionizable lipid is

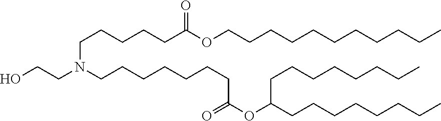 [SMILES: CCCCCCCCCCCOC(=O)CCCCCN(CCO)CCCCCCCC(=O)OC(CCCCCCCC)CCCCCCCC]

or a salt thereof.
E326. The method of any one of E290-E325, wherein the lipid nanoparticle comprises a non-cationic helper lipid.
E327. The method of E326, wherein the non-cationic helper lipid is a phospholipid.
E328. The method of E327, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E329. The method of E328, wherein the phospholipid is DSPC.
E330. The method of E326, wherein the non-cationic helper lipid is oleic acid.
E331. The method of any one of E290-E330, wherein the lipid nanoparticle comprises a PEG-lipid.
E332. The method of E331, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E333. The method of E332, wherein the PEG lipid is selected from the group consisting of PEG-c-DOMG, PEG-DMG, PEG-DLPE, PEG-DMPE, PEG-DPPC and PEG-DSPE lipid.
E334. The method of E333, wherein the PEG lipid is PEG-DMG.
E335. The method of any one of E290-E334, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E336. The method of any one of E290-E335, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E337. The method of any one of E290-E336, wherein the lipid nanoparticle comprises about 50 mol % ionizable lipid, about 10 mol % phospholipid, about 38.5 mol % sterol, and about 1.5 mol % PEG lipid.
E338. The method of any one of E290-E337, wherein the nucleic acid molecule is selected from the group consisting of RNA, mRNA, RNAi, dsRNA, siRNA, antisense RNA, ribozyme, CRISPR/Cas9, ssDNA and DNA.
E339. The method of E338, wherein the nucleic acid is RNA selected from the group consisting of a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.
E340. The method of E339, wherein the nucleic acid is an mRNA.
E341. The method of E340, wherein the mRNA is a modified mRNA comprising one or more modified nucleobases.
E342. The method of E340 or E341, wherein the mRNA includes one or more of a stem loop, a chain terminating nucleoside, a polyA sequence, a polyadenylation signal, and/or a 5′ cap structure.
E343. The method of any one of E340-E342, wherein the mRNA encodes a protein for expression in the immune cell.

### Definitions

As used herein, the term “T cell anergy” refers to a mechanism of peripheral tolerance whereby a T cell enters a hyporesponsive state established upon recognition of antigen in the absence of co-stimulation. Under such conditions, T cells fail to become fully activated and enter a state of unresponsiveness that prevents cell proliferation and cytokine production in response to antigen re-encounter.

“Autoantigens,” as used herein, are normal tissue constituents in the body targeted by an autologous humoral (B cell) or T cell mediated immune response that often results in damage to the tissue and/or autoimmune disease. “Autologous” as used herein refers to cells or tissues derived from the same individual or cells or tissues that are immunologically compatible, e.g., have an identical MHC/HLA haplotypes.

An “autoimmune disorder,” as used herein, refers to a disease state in which, via the action of white blood cells (e.g., B cells, T cells, macrophages, monocytes, or dendritic cells), a pathological immune response (e.g., pathological in duration and/or magnitude) against one or more endogenous antigens, i.e., one or more autoantigens, with consequent tissue damage that may result from direct attack on the cells bearing the one or more autoantigens, from immune-complex formation, or from local inflammation. Autoimmune diseases are characterized by increased inflammation due to immune system activation against self-antigens.

The terms “allograft”, “homograft” and “allogeneic graft” refer to the transplant of an organ or tissue from one individual to another of the same species with a different genotype, including transplants from cadaveric, living related, and living unrelated donors. A graft transplanted from one individual to the same individual is referred to as an “autologous graft” or “autograft”. A graft transplanted between two genetically identical or syngeneic individuals is referred to as a “syngeneic graft”. A graft transplanted between individuals of different species is referred to as a “xenogeneic graft” or “xenograft”.

As used herein the phrase “immune response” or its equivalent “immunological response” refers to the development of a cellular (mediated by antigen-specific T cells or their secretion products) directed against an autoantigen or an related epitope of an autoantigen. A cellular immune response is elicited by the presentation of polypeptide epitopes in association with Class I or Class II MHC molecules, to activate antigen-specific CD4+ T helper cells and/or CD8+ cytotoxic T cells. The response may also involve activation of other components.

As used herein, the term “immune cell” refers to cells that play a role in the immune response, including lymphocytes, such as B cells and T cells; natural killer cells; myeloid cells, such as monocytes, macrophages, eosinophils, mast cells, basophils, and granulocytes.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

“Immunotherapy” refers to the treatment of a subject afflicted with, or at risk of contracting or suffering a recurrence of, a disease by a method comprising inducing, enhancing, suppressing or otherwise modifying an immune response.

A human “at risk of developing an autoimmune disorder” refers to a human with a family history of autoimmune disorders (e.g., a genetic predisposition to one or more inflammatory disorders) or one exposed to one or more autoimmune disorder/autoantibody-inducing conditions. For example, a human exposed to a shiga toxin is at risk for developing typical HUS. Humans with certain cancers (e.g., liquid tumors such as multiple myeloma or chronic lymphocytic leukemia) can pre-dispose patients to developing certain autoimmune hemolytic diseases. For example, PCH can follow a variety of infections (e.g., syphilis) or neoplasms such as non-Hodgkin's lymphoma. In another example, CAD can be associated with HIV infection, Mycoplasma pneumonia infection, non-Hodgkin's lymphoma, or Waldenstrom's macroglobulinemia. In yet another example, autoimmune hemolytic anemia is a well-known complication of human chronic lymphocytic leukemia, approximately 11% of CLL patients with advanced disease will develop AIHA. As many as 30% of CLL may be at risk for developing AIHA. See, e.g., Diehl et al. (1998) Semin Oncol 25(1):80-97 and Gupta et al. (2002) Leukemia 16(10):2092-2095.

A human “suspected of having an autoimmune disorder” is one who presents with one or more symptoms of an autoimmune disorder. Symptoms of autoimmune disorders can vary in severity and type with the particular autoimmune disorder and include, but are not limited to, redness, swelling (e.g., swollen joints), joints that are warm to the touch, joint pain, stiffness, loss of joint function, fever, chills, fatigue, loss of energy, pain, fever, pallor, icterus, urticarial dermal eruption, hemoglobinuria, hemoglobinemia, and anemia (e.g., severe anemia), headaches, loss of appetite, muscle stiffness, insomnia, itchiness, stuffy nose, sneezing, coughing, one or more neurologic symptoms such as dizziness, seizures, or pain. From the above it will be clear that not all humans are “suspected of having an autoimmune disorder.”

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a LNP, “about” may mean+/−5% of the recited value. For instance, a LNP including a lipid component having about 40% of a given compound may include 30-50% of the compound. In another example, delivery to at least about 15% of T cells may include delivery to 10-20% of T cells.

Cancer. As used herein, “cancer” is a condition involving abnormal and/or unregulated cell growth, e.g., a cell having deregulated control of G1 progression. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Enhanced delivery: As used herein, the term “enhanced delivery” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a nucleic acid (e.g., a therapeutic and/or prophylactic mRNA) by a nanoparticle to a target cell of interest (e.g., immune cell) compared to the level of delivery of the nucleic acid (e.g., a therapeutic and/or prophylactic mRNA) by a control nanoparticle to a target cell of interest (e.g., immune cell). For example, “enhanced delivery” by a immune cell delivery potentiating lipid-containing LNP of the disclosure can be evaluated by comparison to the same LNP lacking an immune cell delivery potentiating lipid. The level of delivery of an immune cell delivery potentiating lipid-containing LNP to a particular cell (e.g., immune cell) may be measured by comparing the amount of protein produced in target cells using the phytoserol-containing LNP versus the same LNP lacking the immune cell delivery potentiating lipid (e.g., by mean fluorescence intensity using flow cytometry), comparing the % of target cells transfected using the immune cell delivery potentiating lipid-containing LNP versus the same LNP lacking the immune cell delivery potentiating lipid (e.g., by quantitative flow cytometry), or comparing the amount of therapeutic and/or prophylactic in target cells in vivo using the immune cell delivery potentiating lipid-containing LNP versus the same LNP lacking the immune cell delivery potentiating lipid. It will be understood that the enhanced delivery of a nanoparticle to a target cell need not be determined in a subject being treated, it may be determined in a surrogate such as an animal model (e.g., a mouse or non-human primate model). For example, for determining enhanced delivery to immune cells, a mouse or NHP model (e.g., as described in the Examples) can be used and delivery of an mRNA encoding a protein of interest by a immune cell delivery potentiating lipid-containing LNP can be evaluated in immune cells (e.g., from spleen, peripheral blood and/or bone marrow) (e.g., flow cytometry, fluorescence microscopy and the like) as compared to the same LNP lacking the immune cell delivery potentiating lipid.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a immune cell delivery potentiating lipid in a lipid composition (e.g., LNP) of the disclosure, an effective amount of a immune cell delivery potentiating lipid is an amount sufficient to effect a beneficial or desired result as compared to a lipid composition (e.g., LNP) lacking the immune cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering an immune cell delivery potentiating lipid-containing lipid nanoparticle such that an effective amount of lipid nanoparticles are taken up by immune cells in a subject, an effective amount of immune cell delivery potentiating lipid-containing LNP is an amount sufficient to effect a beneficial or desired result as compared to an LNP lacking the immune cell delivery potentiating lipid. Non-limiting examples of beneficial or desired results in the subject include increasing the percentage of cells transfected, increasing the level of expression of a protein encoded by a nucleic acid associated with/encapsulated by the immune cell delivery potentiating lipid-containing LNP and/or increasing a prophylactic or therapeutic effect in vivo of a nucleic acid, or its encoded protein, associated with/encapsulated by the immune cell delivery potentiating lipid-containing LNP, as compared to an LNP lacking the immune cell delivery potentiating lipid. In some embodiments, a therapeutically effective amount of immune cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of immune cells. For example, an effective amount of immune cell delivery potentiating lipid-containing LNP can be an amount that results in transfection of at least 5%, 10% or 15% of splenic T cells, at least 5%, 10%, 15%, 20% or 25% of splenic B cells, at least 5%, 10%, 15%, 20%, 25%, 30%, 35% or 40% of splenic dendritic cells and/or at least 5% of total bone marrow cells in a mouse model (e.g., as described in Example 9) after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43 S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2): 113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian immune cell) compared to an off-target cell (e.g., non-immune cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model). For example, for determining specific delivery to immune cells, a mouse or NHP model (e.g., as described in the Examples) can be used and delivery of an mRNA encoding a protein of interest can be evaluated in immune cells (e.g., from spleen, peripheral blood and/or bone marrow) as compared to non-immune cells by standard methods (e.g., flow cytometry, fluorescence microscopy and the like).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient. Target immune cells include, for example, CD3+ T cells, CD19+ B cells and CD11c+ dendritic cells, as well as monocytes, tissue macrophages, and bone marrow cells (including immune cells within bone marrow, hematopoietic stem cells, immune cell precursors and fibroblasts).

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning. Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

The Examples demonstrate the physiological effect of immune cell delivery LNPs and were designed to further test the uptake of the subject LNPs by immune cells. These experiments support development of LNPs for delivery of therapeutic molecules for expression in immune cells in patients in vivo or in immune cells from patients ex vivo. The molecules exemplified herein demonstrate the physiological effect of the subject LNPs for delivery of transmembrane molecules (e.g., cellular receptors or modified cellular receptors such as CART), intracellular molecules (e.g., transcription factors), and soluble molecules (e.g., cytokines/growth factors).

### Example 1: Syntheses of Compounds

Syntheses of representative ionizable lipids of the invention, e.g. Compounds having any of Formulae (I), (IA), (II), (IIa), (IIb), (IIc), (IId), (IIe), (III), and (IIIa1-8) and/or any of Compounds X, Y, Z, Q or M are described in co-pending applications PCT/US2016/052352 and PCT/US2016/068300, the contents of each of which are incorporated herein by reference in their entireties.

### Example 2: Production of Nanoparticle Compositions

### A. Production of Nanoparticle Compositions

In order to investigate safe and efficacious nanoparticle compositions for use in the delivery of therapeutic and/or prophylactics to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the therapeutic and/or prophylactic and the other has the lipid components.

Lipid compositions are prepared by combining a lipid according to Formulae (I), (IA), (II), (IIa), (IIb), (IIc), (IId), (IIe), (III), and (IIIa1-8) and/or any of Compounds X, Y, Z, Q or M or a non-cationic helper lipid (such as DOPE, DSPC, or oleic acid obtainable from Avanti Polar Lipids, Alabaster, Ala.), a PEG lipid (such as 1,2-dimyristoyl-sn-glycerol methoxypolyethylene glycol, also known as PEG-DMG, obtainable from Avanti Polar Lipids, Alabaster, Ala.), and a phytosterol (optionally including a structural lipid such as cholesterol) at concentrations of about, e.g., 50 mM in a solvent, e.g., ethanol. Solutions should be refrigeration for storage at, for example, −20° C. Lipids are combined to yield desired molar ratios (see, for example, Table 21 below) and diluted with water and ethanol to a final lipid concentration of e.g., between about 5.5 mM and about 25 mM. Phytosterol* in Table 21 refers to phytosterol or optionally a combination of phytosterol and structural lipid such as beta-phytosterol and cholesterol. Table 21. Exemplary formulations including Compounds according to Formulae (I), (IA), (II), (IIa), (IIb), (IIc), (IId), (IIe), (III), and (IIIa1-8) and/or any of Compounds X, Y, Z, Q or M.

TABLE 21

| Composition (mol %) | Components |
|---|---|
| 40:20:38.5:1.5 | Compound:Phospholipid:Phytosterol*:PEG-DMG |
| 45:15:38.5:1.5 | Compound:Phospholipid:Phytosterol*:PEG-DMG |
| 50:10:38.5:1.5 | Compound:Phospholipid:Phytosterol*:PEG-DMG |
| 55:5:38.5:1.5 | Compound:Phospholipid:Phytosterol*:PEG-DMG |
| 60:5:33.5:1.5 | Compound:Phospholipid:Phytosterol*:PEG-DMG |
| 45:20:33.5:1.5 | Compound:Phospholipid:Phytosterol*:PEG-DMG |
| 50:20:28.5:1.5 | Compound:Phospholipid:Phytosterol*:PEG-DMG |
| 55:20:23.5:1.5 | Compound:Phospholipid:Phytosterol*:PEG-DMG |
| 60:20:18.5:1.5 | Compound:Phospholipid:Phytosterol*:PEG-DMG |
| 40:15:43.5:1.5 | Compound:Phospholipid:Phytosterol*:PEG-DMG |
| 50:15:33.5:1.5 | Compound:Phospholipid:Phytosterol*:PEG-DMG |
| 55:15:28.5:1.5 | Compound:Phospholipid:Phytosterol*:PEG-DMG |
| 60:15:23.5:1.5 | Compound:Phospholipid:Phytosterol*:PEG-DMG |
| 40:10:48.5:1.5 | Compound:Phospholipid:Phytosterol*:PEG-DMG |
| 45:10:43.5:1.5 | Compound:Phospholipid:Phytosterol*:PEG-DMG |
| 55:10:33.5:1.5 | Compound:Phospholipid:Phytosterol*:PEG-DMG |
| 60:10:28.5:1.5 | Compound:Phospholipid:Phytosterol*:PEG-DMG |
| 40:5:53.5:1.5 | Compound:Phospholipid:Phytosterol*:PEG-DMG |
| 45:5:48.5:1.5 | Compound:Phospholipid:Phytosterol*:PEG-DMG |
| 50:5:43.5:1.5 | Compound:Phospholipid:Phytosterol*:PEG-DMG |
| 40:20:40:0 | Compound:Phospholipid:Phytosterol*:PEG-DMG |
| 45:20:35:0 | Compound:Phospholipid:Phytosterol*:PEG-DMG |
| 50:20:30:0 | Compound:Phospholipid:Phytosterol*:PEG-DMG |
| 55:20:25:0 | Compound:Phospholipid:Phytosterol*:PEG-DMG |
| 60:20:20:0 | Compound:Phospholipid:Phytosterol*:PEG-DMG |
| 40:15:45:0 | Compound:Phospholipid:Phytosterol*:PEG-DMG |
| 45:15:40:0 | Compound:Phospholipid:Phytosterol*:PEG-DMG |
| 50:15:35:0 | Compound:Phospholipid:Phytosterol*:PEG-DMG |
| 55:15:30:0 | Compound:Phospholipid:Phytosterol*:PEG-DMG |
| 60:15:25:0 | Compound:Phospholipid:Phytosterol*:PEG-DMG |
| 40:10:50:0 | Compound:Phospholipid:Phytosterol*:PEG-DMG |
| 45:10:45:0 | Compound:Phospholipid:Phytosterol*:PEG-DMG |
| 50:0:48.5:1.5 | Compound:Phospholipid:Phytosterol*:PEG-DMG |
| 50:10:40:0 | Compound:Phospholipid:Phytosterol*:PEG-DMG |
| 55:10:35:0 | Compound:Phospholipid:Phytosterol*:PEG-DMG |
| 60:10:30:0 | Compound:Phospholipid:Phytosterol*:PEG-DMG |

Nanoparticle compositions including a therapeutic and/or prophylactic and a lipid component are prepared by combining the lipid solution with a solution including the therapeutic and/or prophylactic at lipid component to therapeutic and/or prophylactic wt:wt ratios between about 5:1 and about 50:1. The lipid solution is rapidly injected using a NanoAssemblr microfluidic based system at flow rates between about 10 ml/min and about 18 ml/min into the therapeutic and/or prophylactic solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kDa. The first dialysis is carried out at room temperature for 3 hours. The formulations are then dialyzed overnight at 4° C. The resulting nanoparticle suspension is filtered through 0.2 m sterile filters (Sarstedt, Numbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a therapeutic and/or prophylactic (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the nanoparticle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

C. In Vivo Formulation Studies

In order to monitor how effectively various nanoparticle compositions deliver therapeutic and/or prophylactics to targeted cells, different nanoparticle compositions including a particular therapeutic and/or prophylactic (for example, a modified or naturally occurring RNA such as an mRNA) are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a therapeutic and/or prophylactic in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

### Example 3: mRNA Delivery of a Membrane Bound Protein into Human Acute Myelocytic Leukemia Cells Ex Vivo

In a first series of experiments, the ability of beta-sitosterol/cholesterol-containing LNP, as compared to other LNPs, to deliver mRNA into human acute myelocytic leukemia (AML) cells, obtained from a human patient, was tested ex vivo. AML cells were incubated in the presence of mouse OX40L-encoding mRNA (50 ng) formulated into various LNPs, or PBS, at 37° C. Twenty-four hours later, cells were analyzed by flow cytometry for expression of mouse OX40L protein expression. Cells also were examined for mOX40L expression by fluorescence microscopy using a phycoerythrin (PE)-labeled anti-mOX40L antibody. The following LNP formulations were tested: LNP 1, which is composed of Compound X-DSPC-PEG DMG; LNP 2, which is composed of Compound X-DSPC-Compound 428; and LNP 3, which is composed of Compound X-DSPC-beta-sitosterol/cholesterol-PEG DMG (50 mol % Compound X, 10 mol % DSPC, 38.5 mol % beta-sitosterol/cholesterol (28.5 mol % b-sitosterol, 10 mol % cholesterol), and 1.5 mol % PEG DMG.

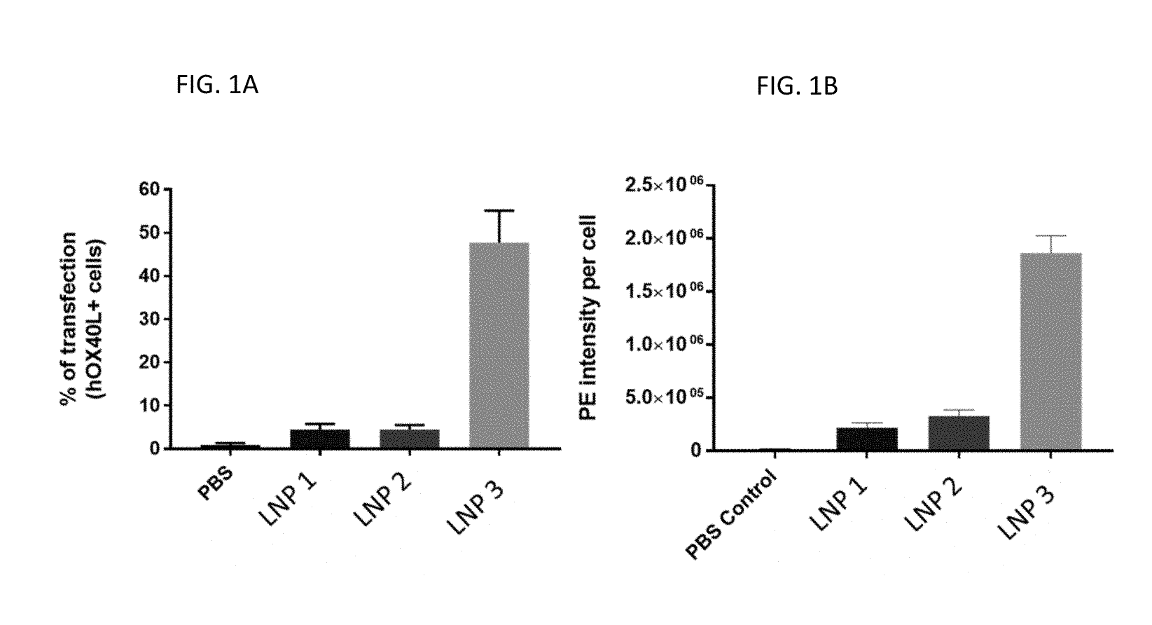

The flow cytometry results are summarized in FIG. 1A, which shows that approximately 50% of the cells treated with the beta-sitosterol/cholesterol-containing LNP (LNP 3) exhibited expression of mOX40L, which was approximately 10-fold higher levels of transfection than observed with the other two LNPs tested (LNP 1 and LNP 2). The fluorescence microscopy results are summarized in FIG. 1B, which confirmed that the cells treated with the beta-sitosterol/cholesterol-containing LNP (LNP 3) exhibited significantly higher levels of expression of mOX40L, as compared to treatment with the other two LNPs tested (LNP 1 and LNP 2).

The results in FIGS. 1A-1B demonstrated that the beta-sitosterol/cholesterol-containing LNP (LNP 3) was significantly better at delivering the mRNA construct into human AML cells ex vivo than the other LNPs tested (LNP 1 and LNP 2). Based on these results, the beta-sitosterol/cholesterol-containing LNP (LNP 3) was tested for in vivo mRNA delivery in an AML mouse model (see Example 4).

### Example 4: mRNA Delivery in PDX Mice Reconstituted with Peripheral Blood Mononuclear Cells (PBMCs) from Acute Myelocytic Leukemia (AML) Patients

In a second series of experiments, a Patient Derived Xenograft (PDX) mouse model of acute myelocytic leukemia (AML) was used to test the ability of various LNP compositions to deliver an mRNA encoding a protein to cells in vivo. In the PDX model, peripheral blood mononuclear cells (PBMCs) from AML patients are administered to immunocompromised mice. The effects of agents can then be tested in the mice.

Five-week-old female NOD.Cg-Prkdcscid Il2rgtm1Sug/JicTac (NOG; Taconic #NOG-F) mice were sublethally irradiated with 175cGy whole body irradiation (RS 2000—X-ray Biological Irradiator, RadSource Technologies, Inc.). In parallel, PBMCs from an AML patient were thawed at 37° C. and viable cells were counted using a hemocytometer. Mice were injected with up to 2×10⁶ human AML cells (0.2 ml volume in PBS) into the lateral tail vein of irradiated mice. When individual animals had between 20-2000 CD33⁺ blasts/μl peripheral blood, an mRNA construct encoding mOX40L formulated (encapsulated) in various different LNP compositions was intravenously injected into the mice. Twenty-four hours post injection, the mice were euthanized and their spleens were collected and analyzed by flow cytometry for expression of the mOX40L protein encoded by the mRNA construct.

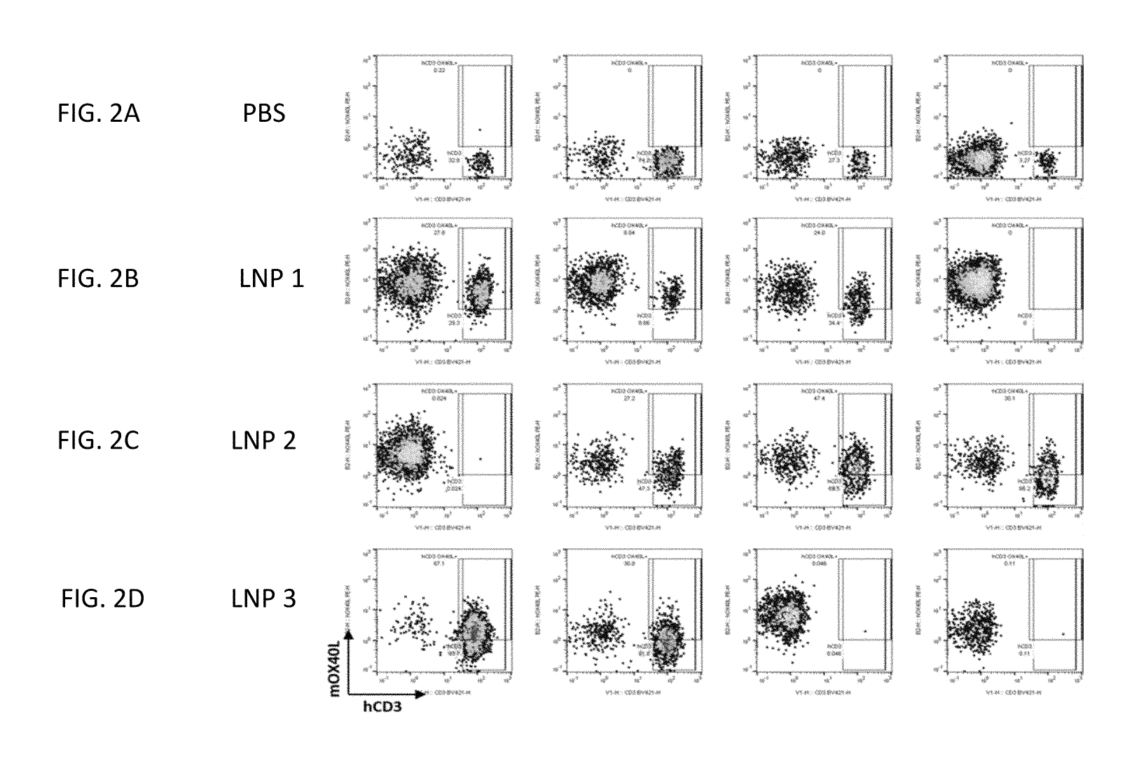

Initial flow cytometry analysis of the splenic cells suggested that a T cell population within the spleen expressed the mOX40L encoded by the mRNA encapsulated by the LNP. To further establish this, double-staining for CD3 and for mOX40L was performed. These flow cytometry results are shown in FIGS. 2A-2D, wherein the X-axis shows cells expressing human CD3 and the Y-axis shows cells expressing mouse OX40L. FIG. 2A shows the results for mice administered PBS as a negative control. FIG. 2B shows the results for mice administered the mOX40L-encoding mRNA construct encapsulated in an LNP containing Compound X-DSPC-PEG DMG (LNP 1). FIG. 2C shows the results for mice administered the mOX40L-encoding mRNA construct encapsulated in an LNP containing Compound X-DSPC-Compound 428 (LNP 2). FIG. 2D shows the results for mice administered the mOX40L-encoding mRNA construct encapsulated in an LNP containing Compound X-beta-sitosterol/cholesterol-DSPC-PEG DMG (LNP 3).

The results in FIGS. 2A-2D demonstrated that the highest levels of hCD3+ cells that also expressed mOX40L (encoded by the mRNA construct encapsulated by the LNP) were observed using the beta-sitosterol/cholesterol-containing LNP (FIG. 2D, upper right quadrants). Thus, these initial results indicated that the beta-sitosterol/cholesterol-containing LNP was effectively delivering the mRNA construct into T cells in vivo.

In repeat confirmatory studies using the PDX mouse model as described above, mOX40L transfection efficiency by the beta-sitosterol/cholesterol-containing LNP (LNP 3) was observed in multiple cell types. In particular, flow cytometry staining demonstrated high levels of mOX40L+ cells among the human CD3+ cell population (human T cells), the human CD33+ cell population (human AML cells) and the mouse CD45+ cell population (mouse leukocytes) (data not shown), thereby confirming transfection of immune cells by the beta-sitosterol/cholesterol-containing LNP in vivo.

### Example 5: mRNA Delivery into Human Peripheral Blood Mononuclear Cells (PBMCs) Ex Vivo

Following the initial in vivo study in the PDX model that indicated the beta-sitosterol/cholesterol-containing LNP was effectively delivering the mRNA construct into T cells in vivo, additional studies were performed ex vivo to further confirm delivery of the mRNA construct into T cells by the LNP.

In this example, human peripheral blood mononuclear cells (PBMCs) from two different Donors (Donor 1 and Donor 2) were incubated in the presence of mRNA encoding mouse OX40L (mOX40L) (20 ng or 50 ng) encapsulated in each of the three different LNP compositions tested in Example 3. PBS was used as a negative control. Human PBMCs were incubated ex vivo with the various LNPs at 37° C. After 24 hours, cells were analyzed by flow cytometry for mOX40L protein expression in CD3+ T cells.

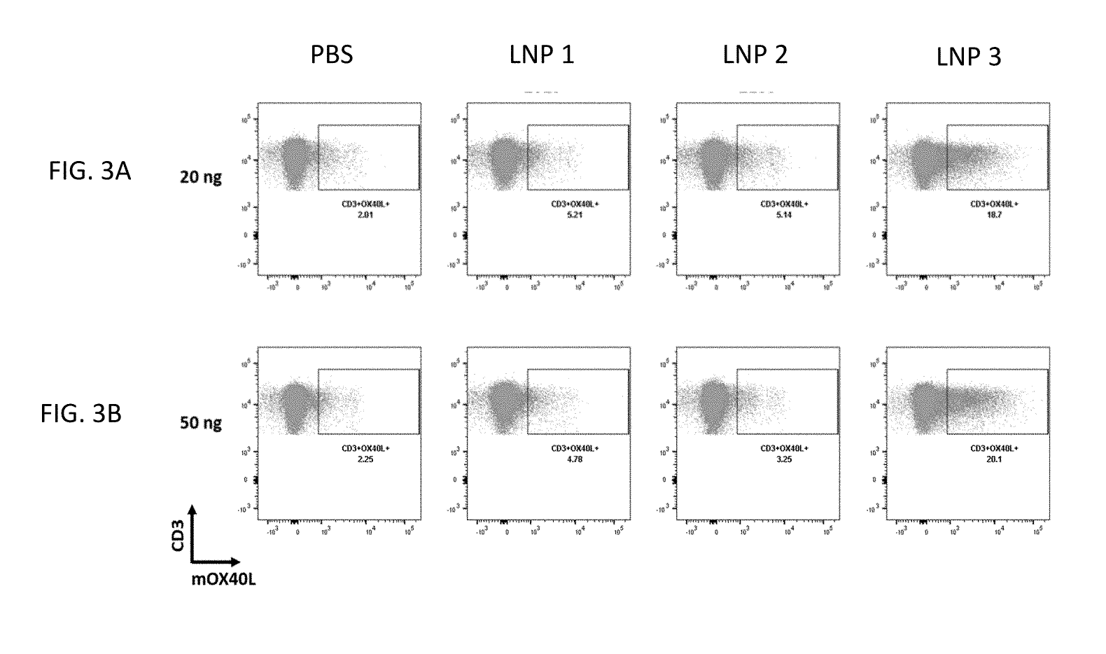

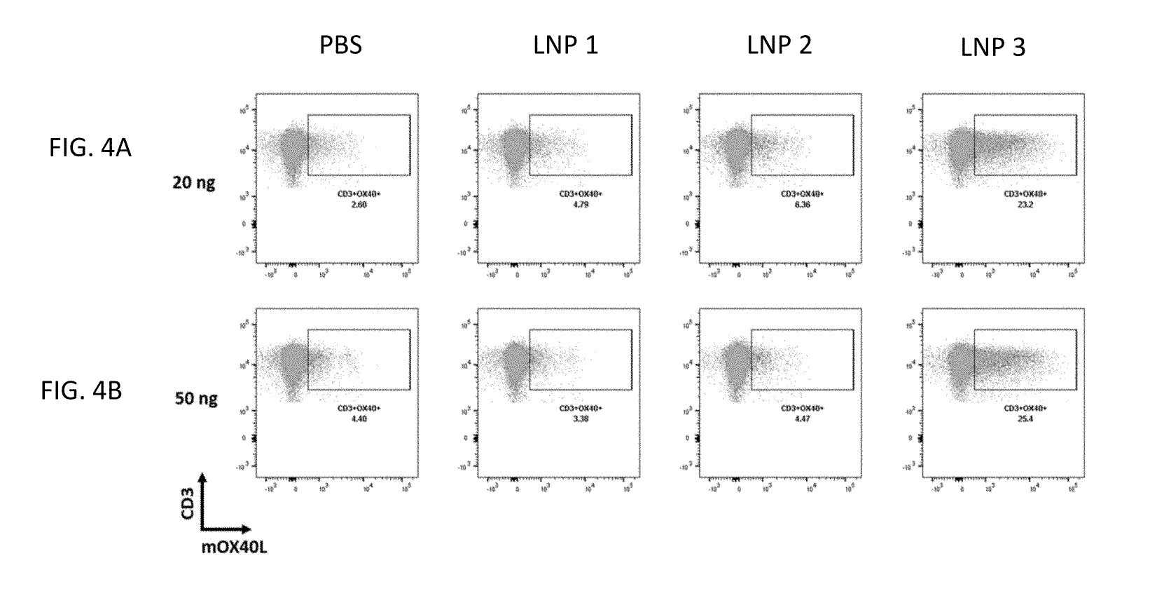

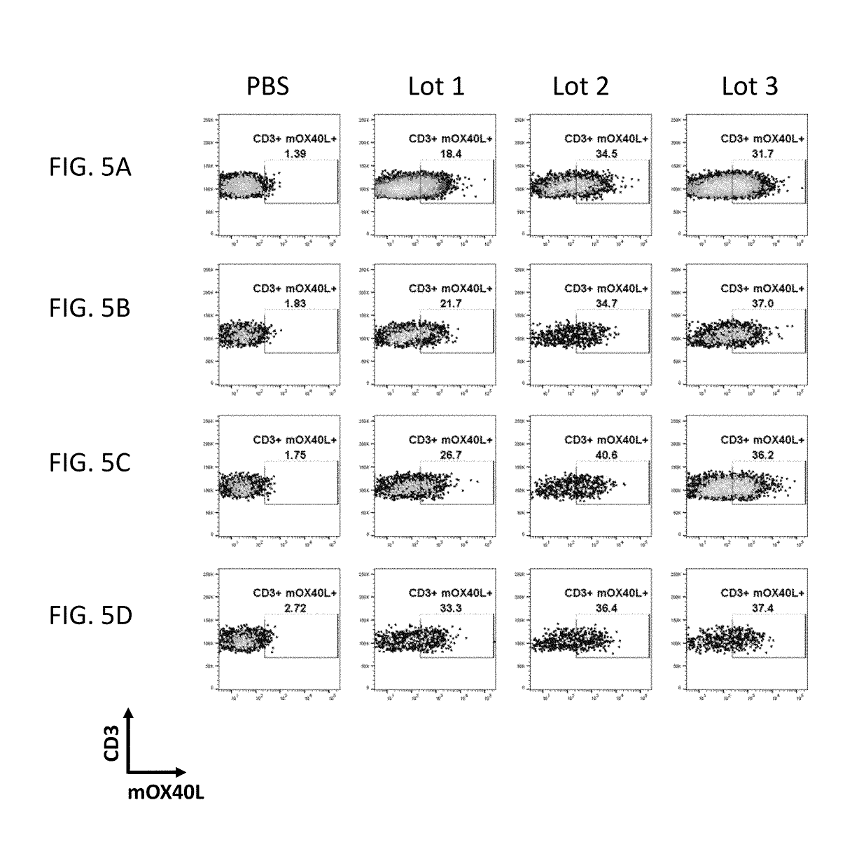

The results for Donor 1 are shown in FIGS. 3A-3B and the results for Donor 2 are shown in FIGS. 4A-4B, wherein FIGS. 3A and 4A show the results using 20 ng of mRNA and FIGS. 3B and 4B show the results using 50 ng of mRNA. For each flow cytometry graph, the X-axis shows cells expressing mOX40L and the Y-axis shows cells expressing human CD3. From left to right, the flow cytometry graphs show the results for mice administered (i) PBS; (ii) an LNP containing Compound X-DSPC-PEG DMG and cholesterol as the structural lipid (LNP 1); (iii) an LNP containing Compound X-DSPC-Compound 428 and cholesterol as the structural lipid (LNP 2); or (iv) an LNP containing Compound X-beta-sitosterol/cholesterol-DSPC-PEG DMG (LNP 3).

The results shown in FIGS. 3A-3B and 4A-4B confirmed that the PBMCs treated with the beta-sitosterol/cholesterol-containing LNP exhibited the highest levels of CD3+ cells that also expressed mOX40L (encoded by the mRNA construct encapsulated by the LNP). Accordingly, this study demonstrated that the beta-sitosterol/cholesterol-containing LNP was effectively delivering the mRNA construct into human peripheral blood T cells ex vivo.

The above study was repeated by transfecting human PBMCs ex vivo with three different lots of the beta-sitosterol/cholesterol-containing LNP encapsulating the mOX40L-encoding mRNA, as compared to the PBS control. The flow cytometry results are shown in FIGS. 5A-5D, with FIGS. 5A, 5B, 5C and 5D representing four technical replicates and the panels (left to right) representing treatment with PBS, Lot 1, Lot 2 or Lot 3 of the LNP, respectively. For each flow cytometry graph, the X-axis shows cells expressing mOX40L and the Y-axis shows cells expressing human CD3. The results in FIGS. 5A-5D confirmed that each of the three different lots of the beta-sitosterol/cholesterol-containing LNP was effective in delivering the mOX40L-encoding mRNA into the CD3+ T cells.

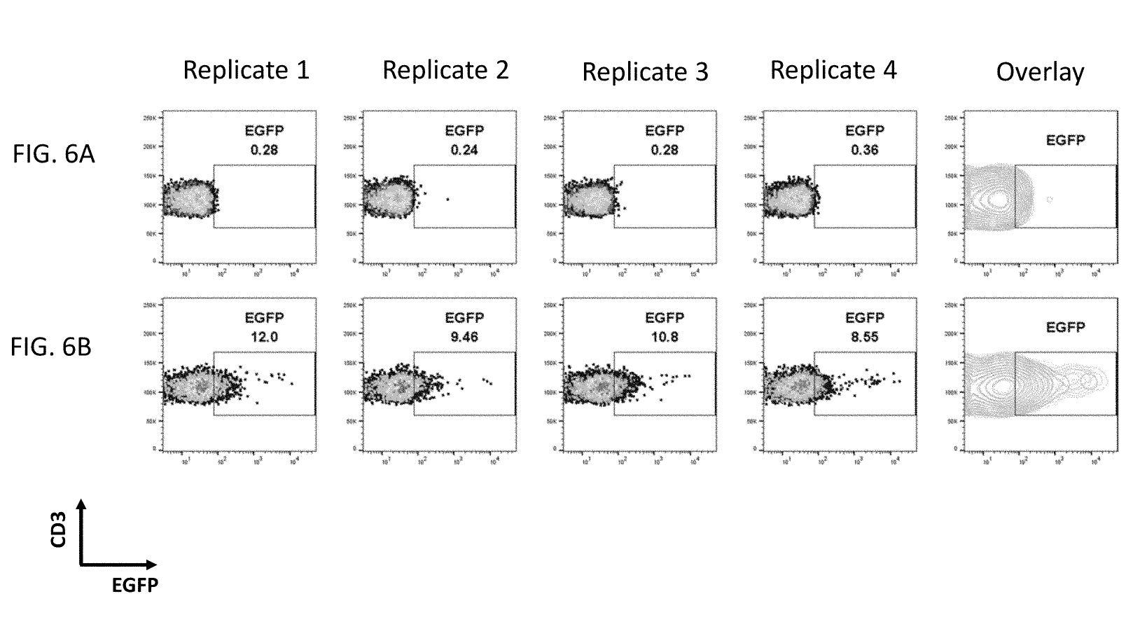

Another study was performed to examine delivery of a different mRNA construct to human PBMCs ex vivo by the beta-sitosterol/cholesterol-containing LNP. Accordingly, for this study an mRNA construct encoding enhanced green fluorescent protein (EGFP) was encapsulated into the LNP and human PBMCs were incubated ex vivo with either the LNP or PBS as described above. After 24 hours, flow cytometry was performed, the results of which are shown in FIGS. 6A-6B, with FIG. 6A showing the results for treatment with PBS and FIG. 6B showing the results for treatment with the beta-sitosterol/cholesterol-containing LNP. The panels, left to right, represent four different replicates and an overlay compilation. For each flow cytometry graph, the X-axis shows cells expressing EGFP and the Y-axis shows cells expressing human CD3. The results demonstrate that the beta-sitosterol/cholesterol-containing LNP effectively delivered this second mRNA construct into the CD3+ T cells.

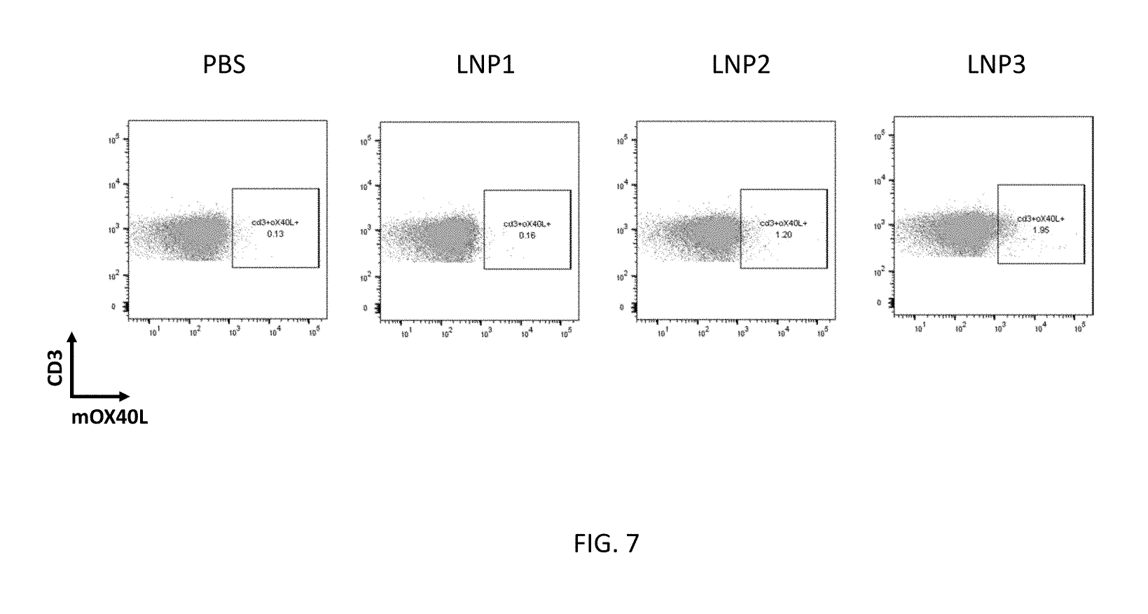

Similar studies were performed to examine delivery of mOX40L-encoding mRNA into mouse PBMCs ex vivo by the beta-sitosterol/cholesterol-containing LNP. Mouse PBMCs were incubated ex vivo in the presence of 50 ng mRNA encapsulated in each of the three different LNP compositions described above (LNP 1, LNP 2, LNP 3) at 37° C. PBS was used as a negative control. After 24 hours, cells were analyzed by flow cytometry for mOX40L protein expression in CD3+ T cells. The results are shown in FIG. 7, which demonstrate that mouse T cells were not transfected ex vivo by any of the LNPs tested, including the beta-sitosterol/cholesterol-containing LNP.

### Example 6: mRNA Delivery into Various Different Immune Cells within Human Peripheral Blood Mononuclear Cells (PBMCs) Ex Vivo

In this example, transfection of different immune cell populations within human peripheral blood mononuclear cells (PBMCs) by the beta-sitosterol/cholesterol-containing LNP (encapsulating mOX40L) was examined by flow cytometry. PMBCs from four different Donors (Donors 1-4) were used. PBS was used as a negative control. Transfections were carried out as described in Example 5. The following cell populations were examined by gating for expression of the following cell surface marker(s): (i) activated NK cells—CD56^{DIM}; (ii) immature NK cells—CD56^{HIGH}; (iii) dendritic cells (DC)—CD11c+, CD11b−; (iv) monocytes—CD11c+, CD11b+; (v) T cells—CD3+; (vi) NK T cells—CD3+, CD56+; and (vii) B cells—CD19+.

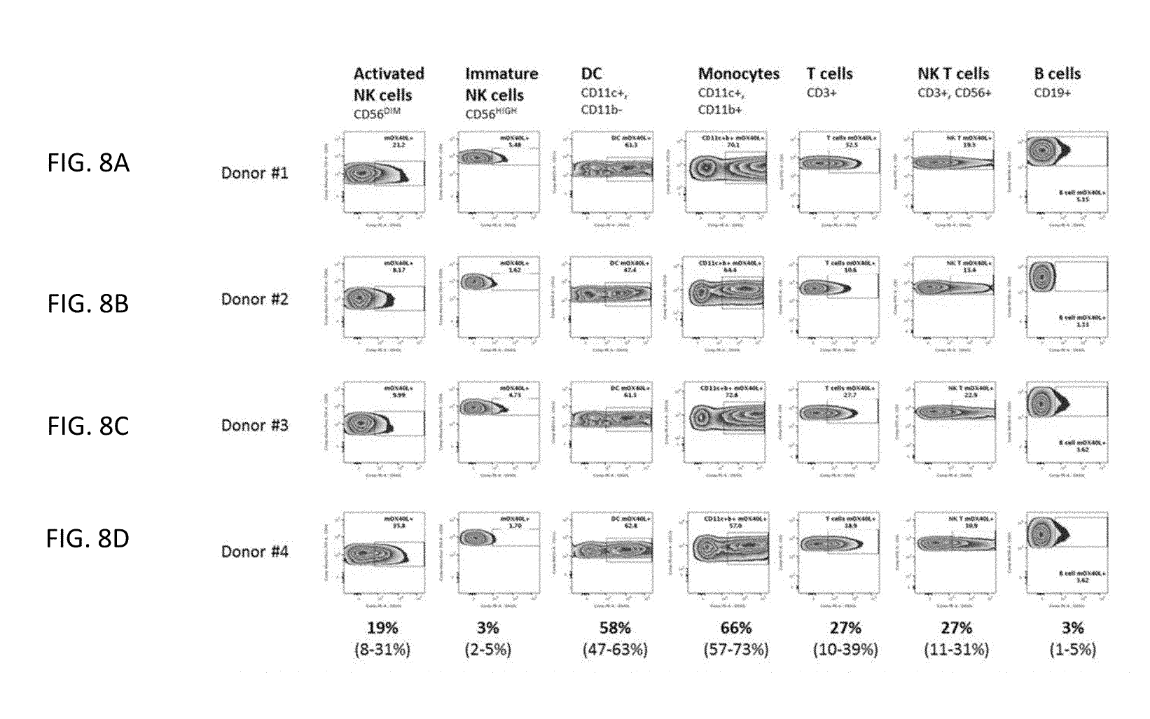

The results for Donors 1-4 are shown in FIGS. 8A-8D, respectively. The negative control (PBS) exhibited less than 1% transfection for all cell subsets examined (data not shown). In contrast, all seven human immune cell types examined exhibited transfection ex vivo using the beta-sitosterol/cholesterol-containing LNP. In particular, activated NK cells exhibited 19% (8-31%) transfection, immature NK cells exhibited 3% (2-5%) transfection, DCs exhibited 58% (47-63%) transfection, monocytes exhibited 66% (57-73%) transfection, T cells exhibited 27% (10-39%) transfection, NK T cells exhibited 27% (11-31%) transfection and B cells exhibited 3% (1-5%) transfection.

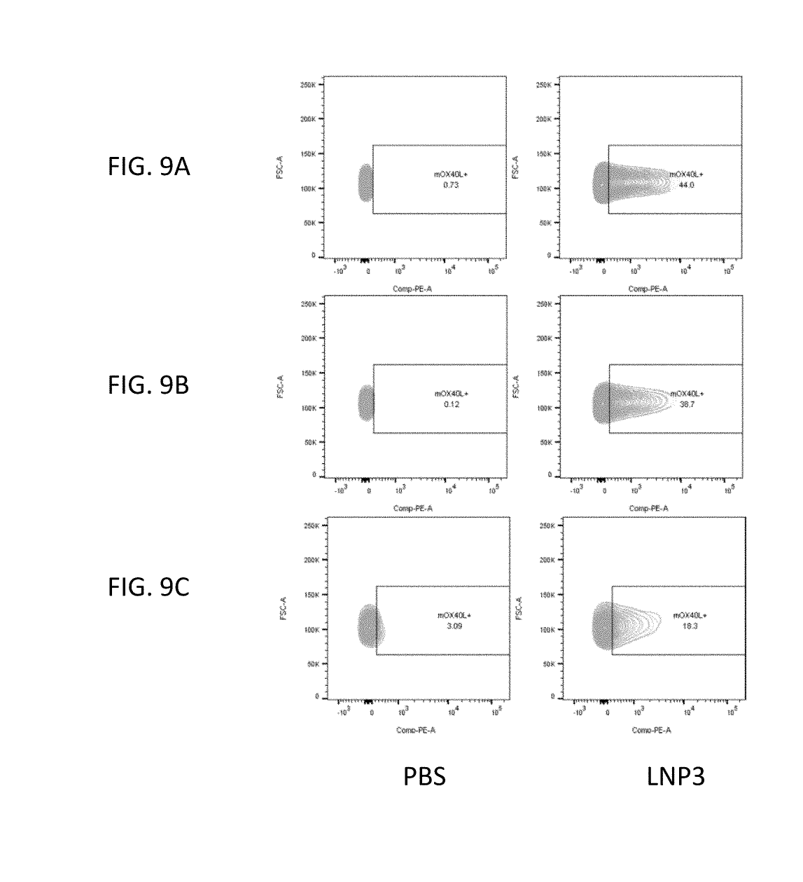

In additional studies, transfection ex vivo of different subsets of T cells within the human CD3+ T cell population by the beta-sitosterol/cholesterol-containing LNP (LNP 3, encapsulating mOX40L) was examined by flow cytometry. PBS was used as a negative control. Transfections were carried out as described in Example 5. The following cell populations were examined by gating for expression of the indicated cell surface marker(s): (i) CD3+ T cells (total T cells); (ii) CD4+ T cells; (iii) CD8+ T cells; and (iv) CD4+CD25+CD127^{low} Treg cells. The results for CD4+ T cells, CD8+ T cells and Tregs are shown in FIGS. 9A-9C, respectively. The results show that all T cell subsets examined within the total T cell (CD3+) population were effectively transfected using the beta-sitosterol/cholesterol-containing LNP, with the CD4+ and CD8+ T cells exhibited a higher percentage of transfected cells than the Tregs.

Upon repeat transfection of human PBMCs ex vivo, no impact on cell viability was observed. Moreover, no effect on cytokine production, activation or proliferation was observed. These results indicate that enhancement of mRNA delivery by the beta-sitosterol/cholesterol-containing LNP did not cause any deleterious effects on cell viability, cytokine production, activation or proliferation.

### Example 7: mRNA Delivery in Non-Human Primates In Vivo

In this example, the ability of the beta-sitosterol/cholesterol-containing LNP, as compared to other LNPs, to deliver mRNA into non-human primate (NHP) T cells in vivo was tested. Mouse OX40L-encoding mRNA was formulated into various LNPs, which were then administered to NHPs in one 60-minute intravenous infusion at a dose rate of 5 mL/kg/hour via an appropriate peripheral vein on Day 1 of the study. The dose volume for each animal was based on the most recent body weight measurement. The dose was given using a temporary indwelling catheter inserted in a peripheral vein and an injection set.

The experimental design is summarized in Table 22 below.

TABLE 22
Experimental Design

| | | Dose | Dose | Dose | |
|---|---|---|---|---|---|
| Group | LNP Composition | Level | Volume | Conc. | No. of |
| No. | (Number) | (mg/kg) | (mL/kg) | (mg/mL) | Males |
| 1 | Compound X/ | 0.3 | 5 | 0.06 | 2 |
| | cholesterol/DSPC/ | | | | |
| | PEG DMG (LNP 1) | | | | |
| 2 | Compound X/ | 0.3 | 5 | 0.06 | 2 |
| | Cholesterol/DSPC/ | | | | |
| | Compound | | | | |
| | 428 (LNP 2) | | | | |
| 3 | Compound X/ | 0.3 | 5 | 0.06 | 2 |
| | Cholesterol/DSPE/ | | | | |
| | PEG (LNP 4) | | | | |
| 4 | Compound X/beta- | 0.3 | 5 | 0.06 | 2 |
| | sitosterol/ | | | | |
| | cholesterol/ | | | | |
| | DSPC/PEG | | | | |
| | DMG (LNP 3) | | | | |

Twenty-four hours after intravenous injection, spleens were harvested, dissociated and single cell solutions were analyzed by flow cytometry. Bone marrow cells from femur and humerus also were harvested and, after flushing a single cell solution, were analyzed by flow cytometry.

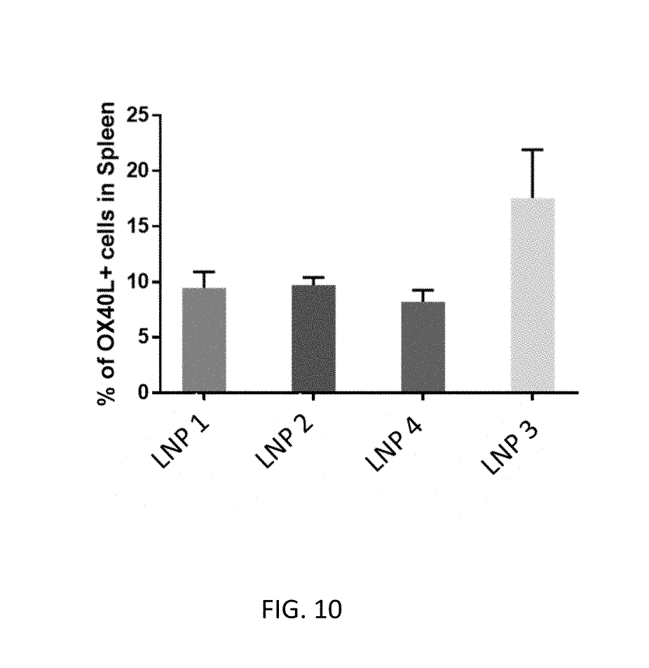

FIG. 10 shows the percentage of mOX40L+ cells in the spleen, demonstrating that animals treated with the beta-sitosterol/cholesterol-containing LNP exhibited a significantly higher percentage of mOX40L+ cells in the spleen as compared to the animals treated with the three other LNPs. These results indicated that the beta-sitosterol/cholesterol-containing LNP was effectively delivering the mRNA into immune cells in the spleen. To examine which types of immune cells in the spleen were being transfected in vivo, additional flow cytometry studies were done, gating for co-expression of mOX40L and either CD3 (for splenic T cells), CD20 (for splenic B cells) or CD11c (for splenic dendritic cells).

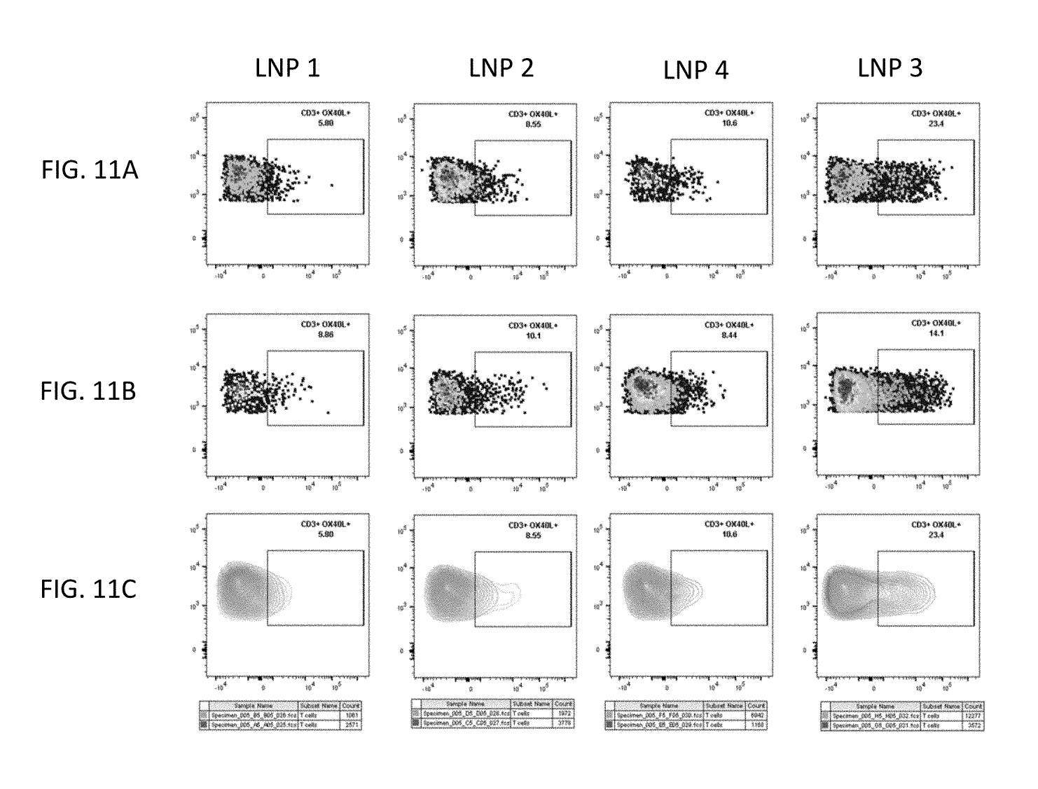

The flow cytometry results for mOX40L+ CD3+ splenic T cells are shown in FIGS. 11A-11C, wherein FIGS. 11A and 11B show the results from two different animals and FIG. 11C shows an overlay composite of the two. The panels (left to right) show the results for treatments groups 1, 2, 3 and 4, respectively. For each flow cytometry graph, the X-axis shows cells expressing mOX40L and the Y-axis shows cells expressing CD3. The results in FIGS. 11A-11C demonstrated that the beta-sitosterol/cholesterol-containing LNP was significantly better at delivering the mRNA construct into NHP splenic T cells in vivo than any of the three other LNPs tested.

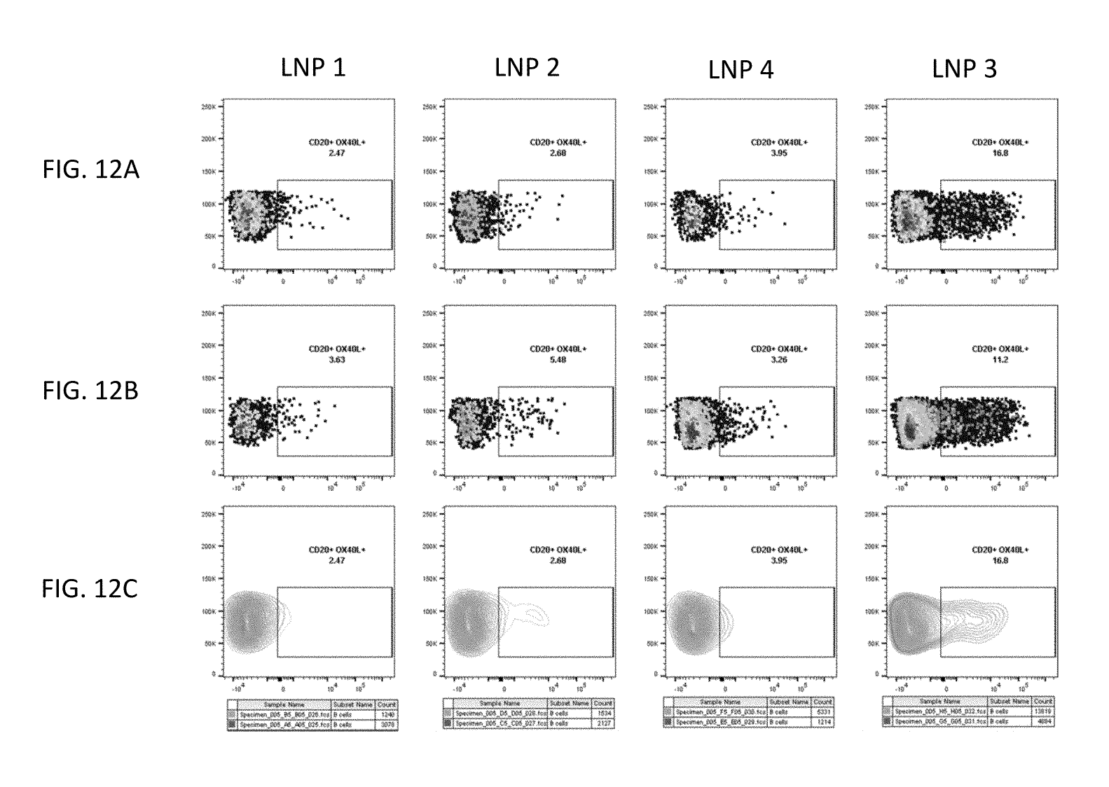

The flow cytometry results for mOX40L+ CD20+ splenic B cells are shown in FIGS. 12A-12C, wherein FIGS. 12A and 12B show the results from two different animals and FIG. 12C shows an overlay composite of the two. The panels (left to right) show the results for treatments groups 1, 2, 3 and 4, respectively. For each flow cytometry graph, the X-axis shows cells expressing mOX40L and the Y-axis shows cells expressing CD20. The results in FIGS. 12A-12C demonstrated that the beta-sitosterol/cholesterol-containing LNP was significantly better at delivering the mRNA construct into NHP splenic B cells in vivo than any of the three other LNPs tested.

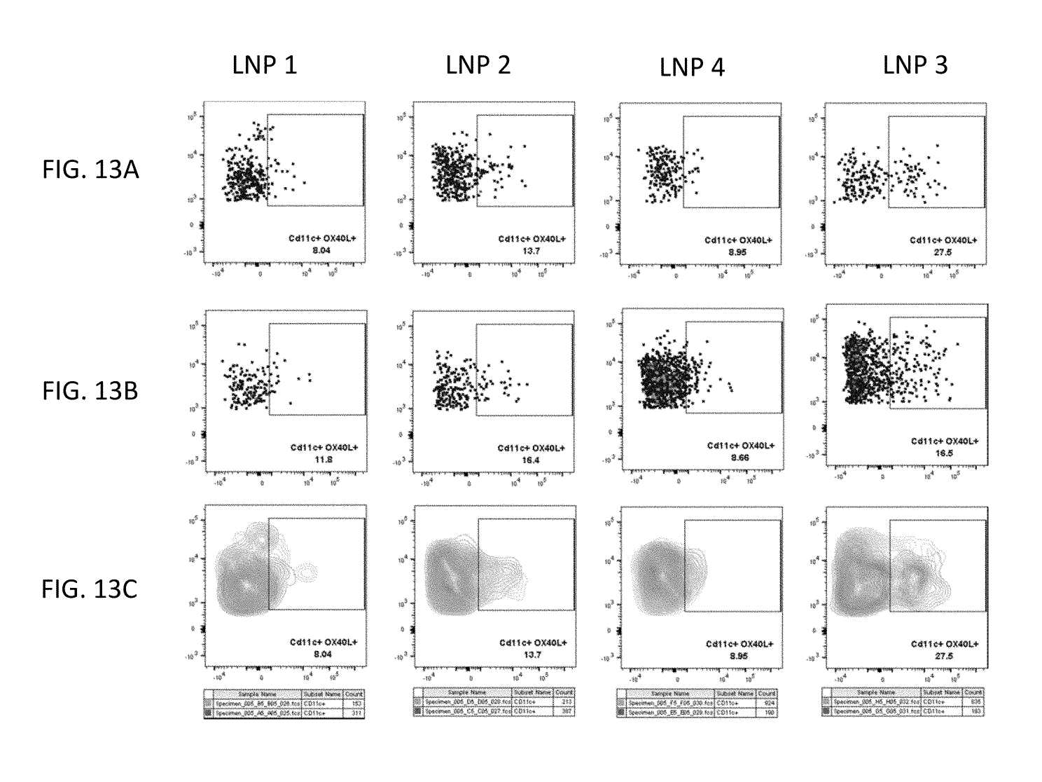

The flow cytometry results for mOX40L+ CD11c+ splenic dendritic cells are shown in FIGS. 13A-13C, wherein FIGS. 13A and 13B show the results from two different animals and FIG. 13C shows an overlay composite of the two. The panels (left to right) show the results for treatments groups 1, 2, 3 and 4, respectively. For each flow cytometry graph, the X-axis shows cells expressing mOX40L and the Y-axis shows cells expressing CD11c. The results in FIGS. 13A-13C demonstrated that the beta-sitosterol/cholesterol-containing LNP was significantly better at delivering the mRNA construct into NHP splenic dendritic cells in vivo than any of the three other LNPs tested.

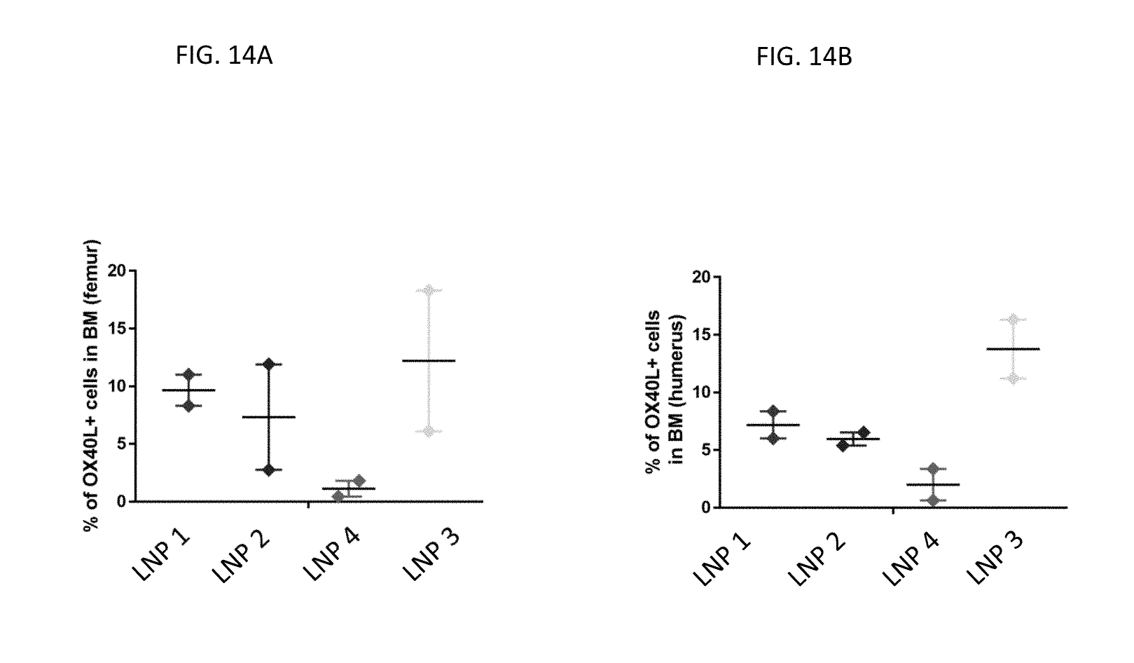

The flow cytometry results for mOX40L+ bone marrow cells are shown in FIGS. 14A-14B, wherein FIG. 14A shows the % of mOX40L+ cells in the bone marrow from the femur and FIG. 14B shows the % of mOX40L+ cells in the bone marrow from the humerus. The results in FIGS. 14A-14B demonstrated that the beta-sitosterol/cholesterol-containing LNP was significantly better at delivering the mRNA construct into NHP bone marrow cells (in particular for humerus bone marrow) in vivo than any of the three other LNPs tested.

Accordingly, this study demonstrated that the beta-sitosterol/cholesterol-containing LNP effectively delivered a protein-encoding mRNA construct into non-human primate splenic cells, resulting in significantly higher levels of protein expression in multiple different types of immune cells within the spleen (T cells, B cells, dendritic cells), as well as effectively delivering the mRNA construct into non-human primate bone marrow cells, as compared to the other LNPs tested.

### Example 8: Confirmatory In Vivo Studies in Non-Human Primates

In this example, confirmatory studies were performed in non-human primates (cynomolgus monkeys) to further examine the delivery of mRNA to immune cells in vivo by the beta-sitosterol/cholesterol-containing LNP. Two sequential studies, similar to that described in Example 7, were performed with separate preparations of material (n=4). Animals were dosed at 0.2 mg/kg intravenously and followed for twenty-four hours prior to necropsy. Animals were treated with mOX40L mRNA encapsulated in LNP 3 (Compound X/beta-sitosterol/cholesterol/PEG DMG). PBS and LNP 1 (Compound X/PEG DMG) were used as controls. Complete histopathology of the hematopoietic system, including bone marrow (two sites), spleen, thymus and lymph nodes, as well as liver, was performed. No adverse clinical observations were reported for either study.

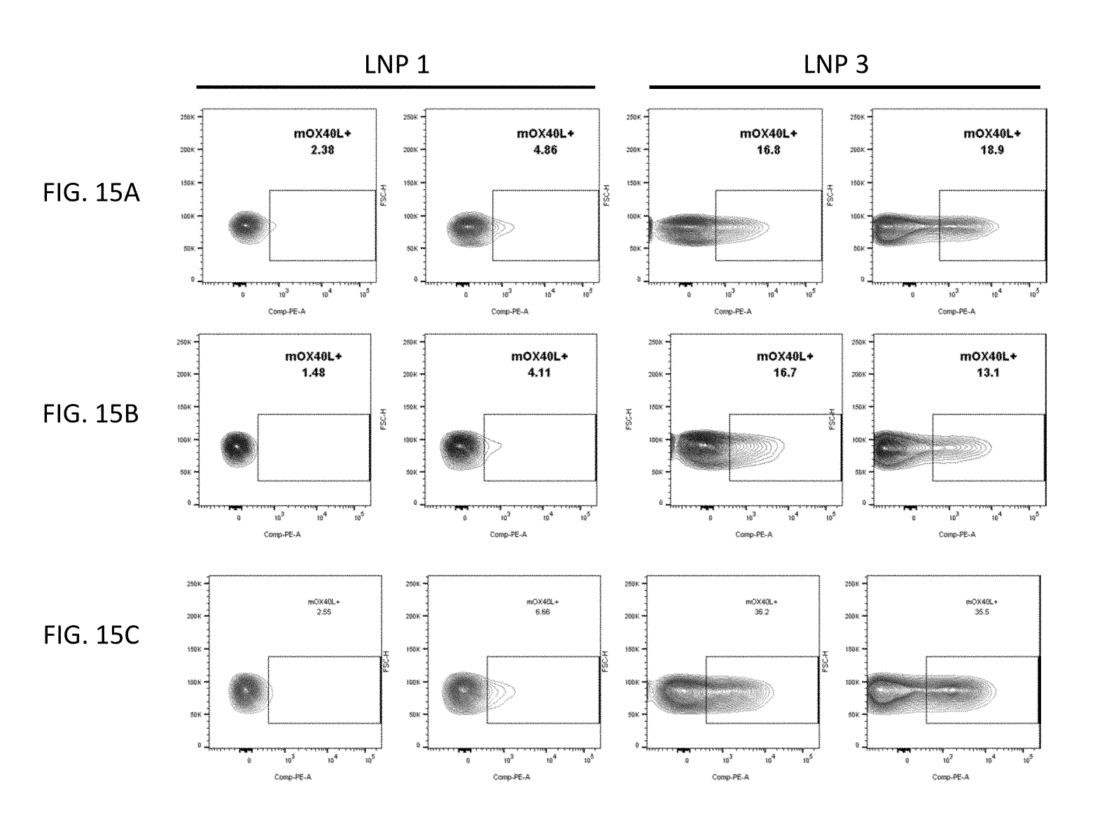

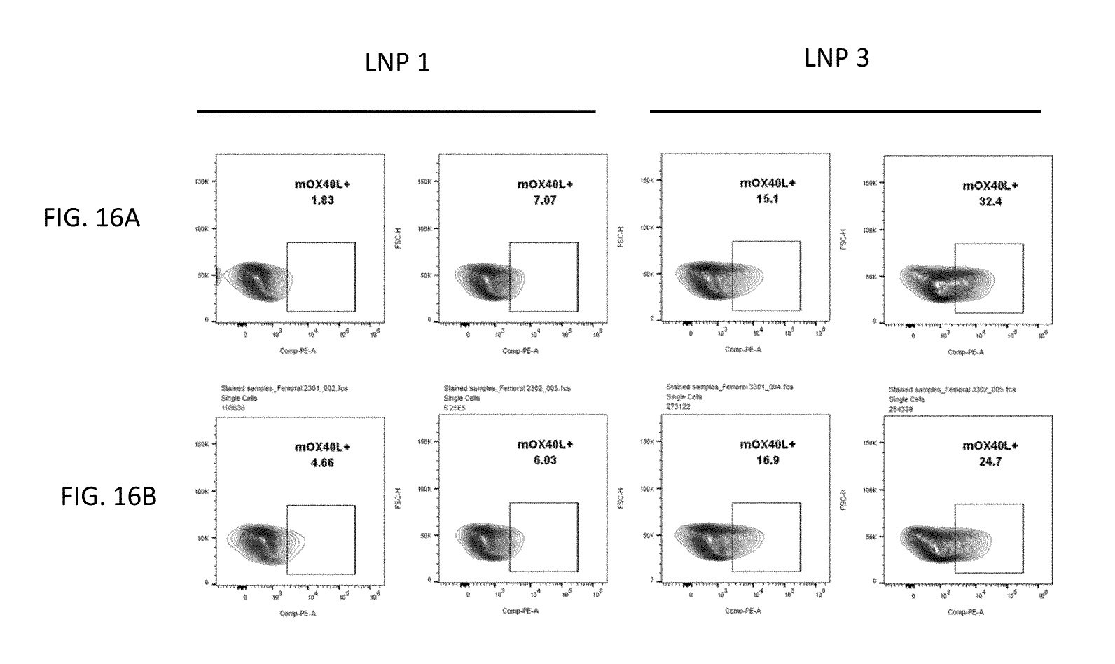

Histological staining confirmed expression in spleen and bone marrow, while no expression was seen in lymph node or thymus at 24 hours (data not shown). Expression in splenic B cells (CD20+), T cells (CD3+) and dendritic cells (CD11c+) was examined by flow cytometry, the results of which are shown in FIGS. 15A-15C. These results demonstrated that use of LNP 3 led to significantly higher levels of expression of the mRNA as compared to LNP 1 in B cells (FIG. 15A; 17% vs. 3%), T cells (FIG. 15B; 15% vs. 3%) and dendritic cells (FIG. 15C; 36% vs. 5%). Bone marrow expression in femur and humerus also was examined by flow cytometry, the results of which are shown in FIGS. 16A-16B. These results demonstrated that use of LNP 3 led to significantly higher levels of expression of the mRNA as compared to LNP 1 in femoral bone marrow (FIG. 16A; 24% vs. 4%) and humeral bone marrow (FIG. 16B; 21% vs. 5%).

Immunophenotyping of bone marrow cells determined that a variety of different immune cells within bone marrow were positive for expression of the transfected mRNA. In particular, approximately 30% of monocytes, approximately 15% of plasma cells and approximately 5% of early progenitors (including proerythroblasts, monoblasts/myeloblasts and mesenchymal stem cells) were positive for expression, whereas less than 5% of immature B cells or B cell precursors exhibited expression.

### Example 9: mRNA Delivery into Non-Human Primate Peripheral Blood Mononuclear Cells (PBMCs) Ex Vivo

In this example, the ability of the beta-sitosterol/cholesterol-containing LNP, as compared to other LNPs, to deliver mRNA into non-human primate (NHP) PBMCs was tested ex vivo. NHP PBMCs were incubated in the presence of mOX40L-encoding mRNA formulated into various LNPs, or PBS, at 37° C. Twenty-four hours later, cells were analyzed by flow cytometry for mOX40L protein expression in CD3+ T cells.

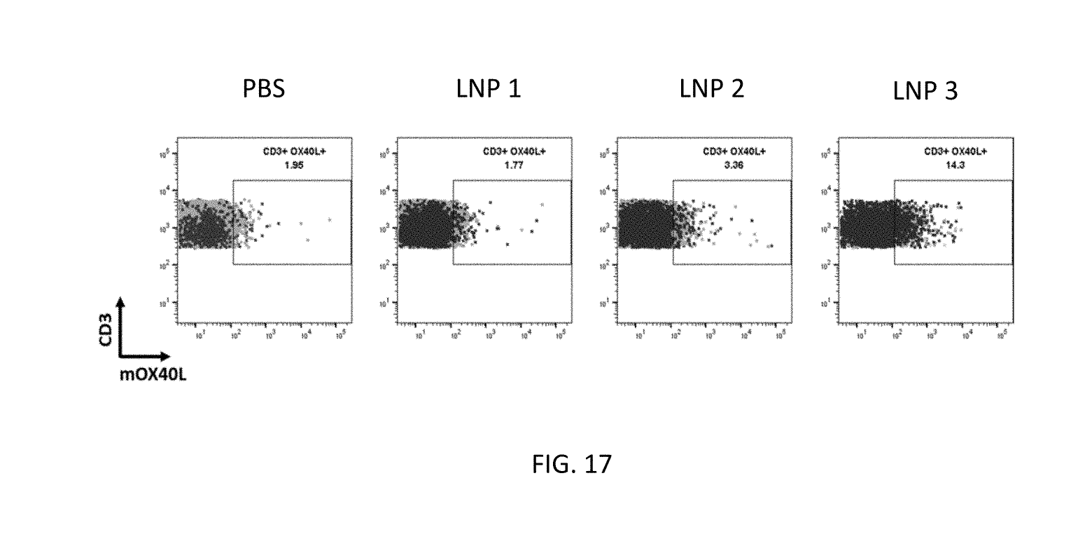

The results are shown in FIG. 17. As indicated, the panels (left to right) show PBMCs incubated with (i) PBS or LNPs containing (ii) Compound X/PEG DMG (LNP 1), (iii) Compound X/Compound 428 (LNP 2) or (iv) Compound X/beta-sitosterol/cholesterol/PEG DMG (LNP 3), respectively. For each flow cytometry graph, the X-axis shows cells expressing mOX40L and the Y-axis shows cells expressing CD3.

The results in FIG. 17 demonstrated that the beta-sitosterol/cholesterol-containing LNP was significantly better at delivering the mRNA construct into NHP PBMC T cells ex vivo than the other LNPs tested.

### Example 10: mRNA Delivery into Human Bone Marrow Plasma Cells Ex Vivo

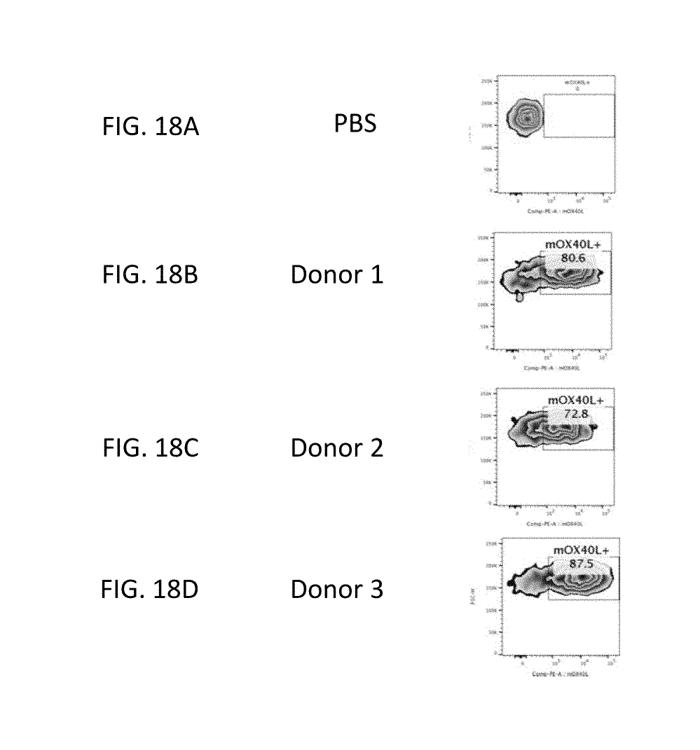

The transfection of bone marrow plasma cells that was observed in non-human primates in vivo, as described in Example 8, was further examined using human bone marrow samples for ex vivo transfection. Five human donor bone marrow samples were transfected with mOX40L-encoding mRNA formulated in the beta-sitosterol/cholesterol-containing LNP (LNP 3). PBS was used as a control. Flow cytometry results for CD45+CD38+CD138+CD19+CD20− plasma cells from three representative donor samples are shown in FIGS. 18A-18D (FIG. 18A shows PBS control, FIGS. 18B-18D show donors 1-3). Similar results were seen for the two additional donors (data not shown). The results demonstrate that approximately 60-80% of the plasma cells were transfected using LNP 3, confirming the ability of LNP 3 to effectively transfect human bone marrow plasma cells.

### Example 11: Pharmacological Profile of LNP 3 in Rats

In this example, the beta-sitosterol/cholesterol-containing LNP (LNP 3) was used to deliver an mOX40L-encoding mRNA to splenic cells in rats in vivo at various doses and using different treatment regimens to examine the pharmacological profile of LNP 3.

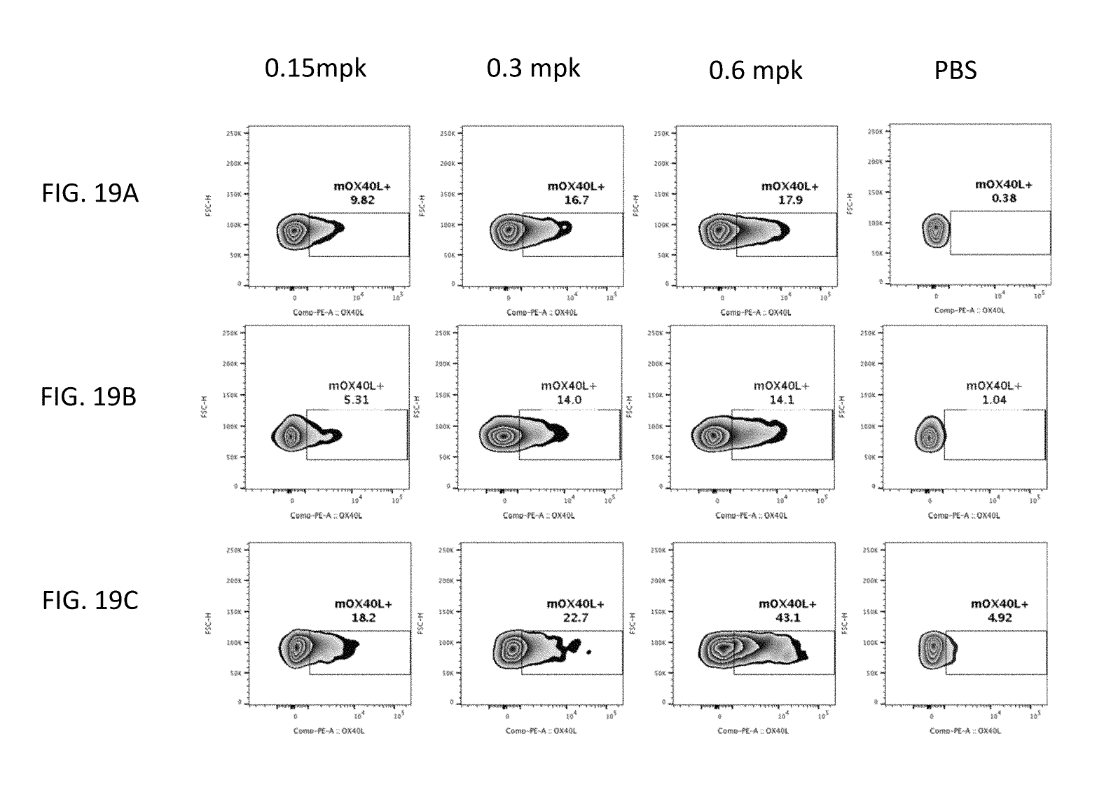

In a first series of experiments, rats were dosed intravenously with either PBS control or mOX40L mRNA-encapsulated LNP 3 at 0.15 mg/kg, 0.3 mg/kg or 0.6 mg/kg. Expression of the transfected mRNA in CD3+ splenic T cells, CD19+ splenic B cells or CD11b+ splenic macrophages was examined by flow cytometry. The results are shown in FIGS. 19A-19C, which demonstrate dose-dependent pharmacology in the rats with possible saturation above 0.3 mg/kg (i.e., transfection levels were approximately equivalent for 0.3 mg/kg and 0.6 mg/kg, but were lower for 0.15 mg/kg).

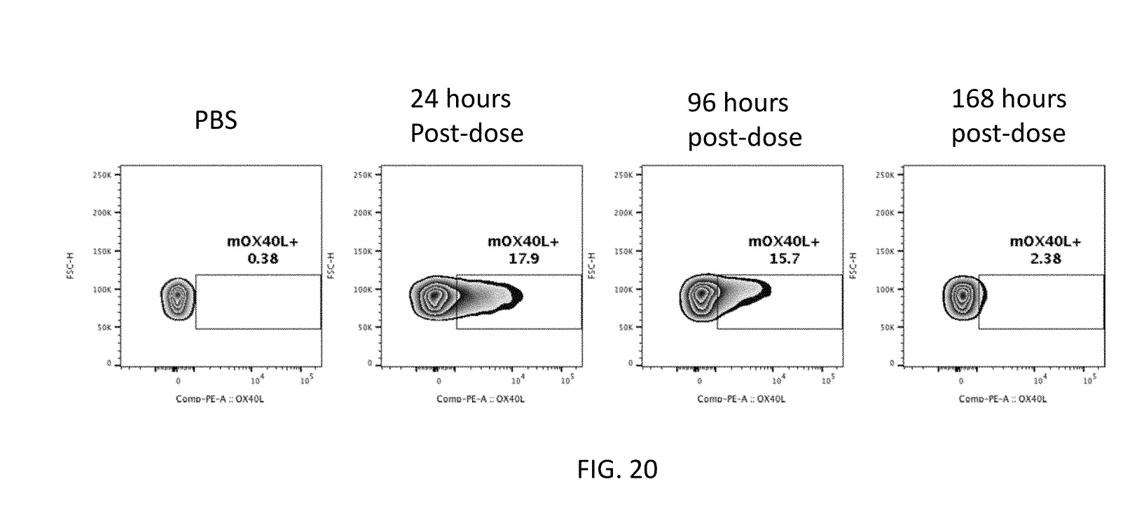

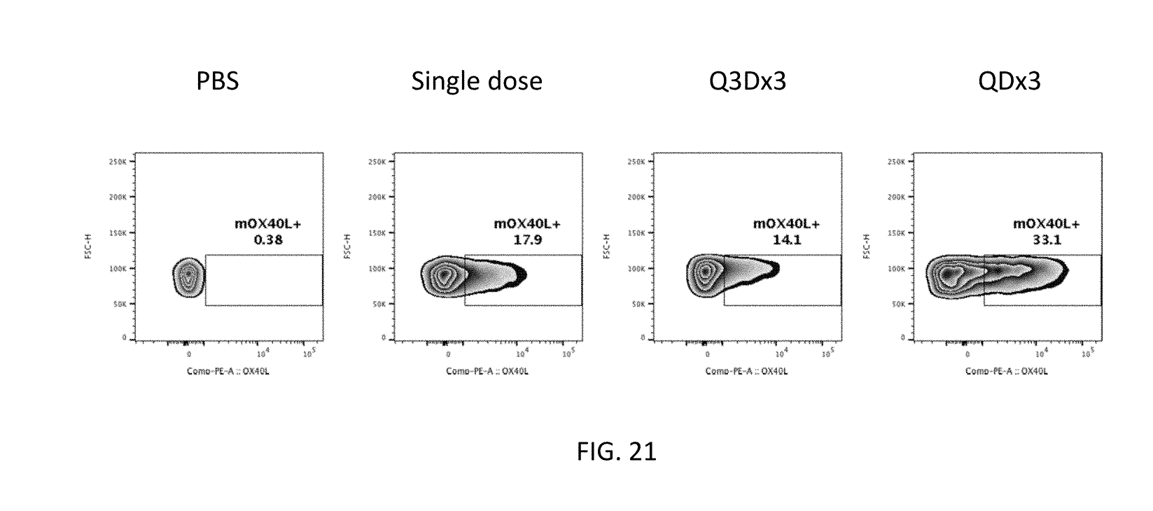

In a second series of experiments, rats were dosed intravenously with either PBS control or mOX40L mRNA-encapsulated LNP 3 at 0.6 mg/kg. The percentage of mOX40L positive splenic cells was determined 24 hours, 96 hours and 168 hours post-dosing. The results are shown in FIG. 20, which demonstrated approximately equivalent percentage of cells transfected at 24 and 96 hours post-dosing, whereas a lower percentage of cells transfected was observed at 168 hours post-dosing.

In a third series of experiments, rats were dosed intravenously with either PBS control or mOX40L mRNA-encapsulated LNP 3 at 0.6 mg/kg. Rats were treated with either a single dose (SD), with one dose a day for three days for a total of three doses (QDx3) or with one dose every 3 days for a total of three doses (Q3Dx3). The percentage of mOX40L positive splenic cells was determined 24 hours after the last dose. The results are shown in FIG. 21, which demonstrated that all dosing regimens resulted in transfection of splenic cells, with the QDx3 regimen leading to the highest percentage of transfected cells.

### Example 12: Effect of Repeat Transfections in Human PBMCs

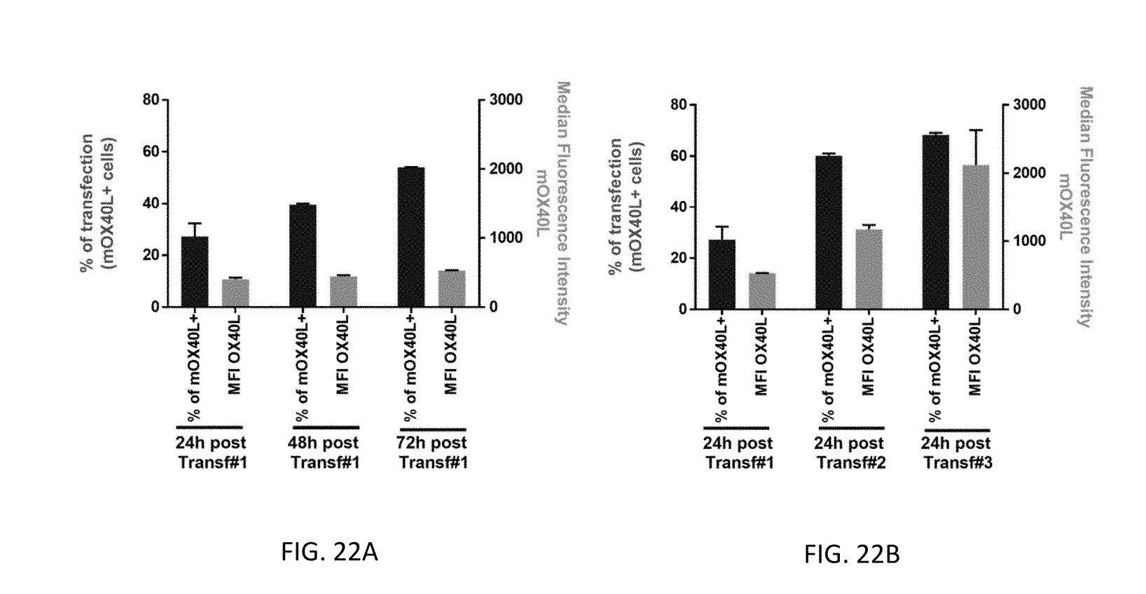

In this example, human PBMCs were transfected ex vivo with the beta-sitosterol/cholesterol-containing LNP (LNP 3) encapsulating mOX40L-encoding mRNA and the effect of repeated transfections of the cells was compared to a single transfection. Cells were transfected as descried in Example 5. For the single transfection sample, the % mOX40L+ cells and Median Fluorescence Intensity (MFI) were measured 24, 48 and 72 hours after transfection. For the multiple transfection sample, the cells were transfected three times and the % mOX40L+ cells and MFI were measured 24 hours after each transfection. The single transfection results are shown in FIG. 22A and the multiple transfection results are shown in FIG. 22B, with % transfection on the left axis and MFI on the right axis. The results demonstrate that repeat transfection of the human PBMCs resulted in a dose-related increase in both % transfection and MFI. Cell viability was not impacted by either the single transfection of the repeated transfections.

### Example 13: Time of LNP Interaction to Transfect Human T Cells Ex Vivo

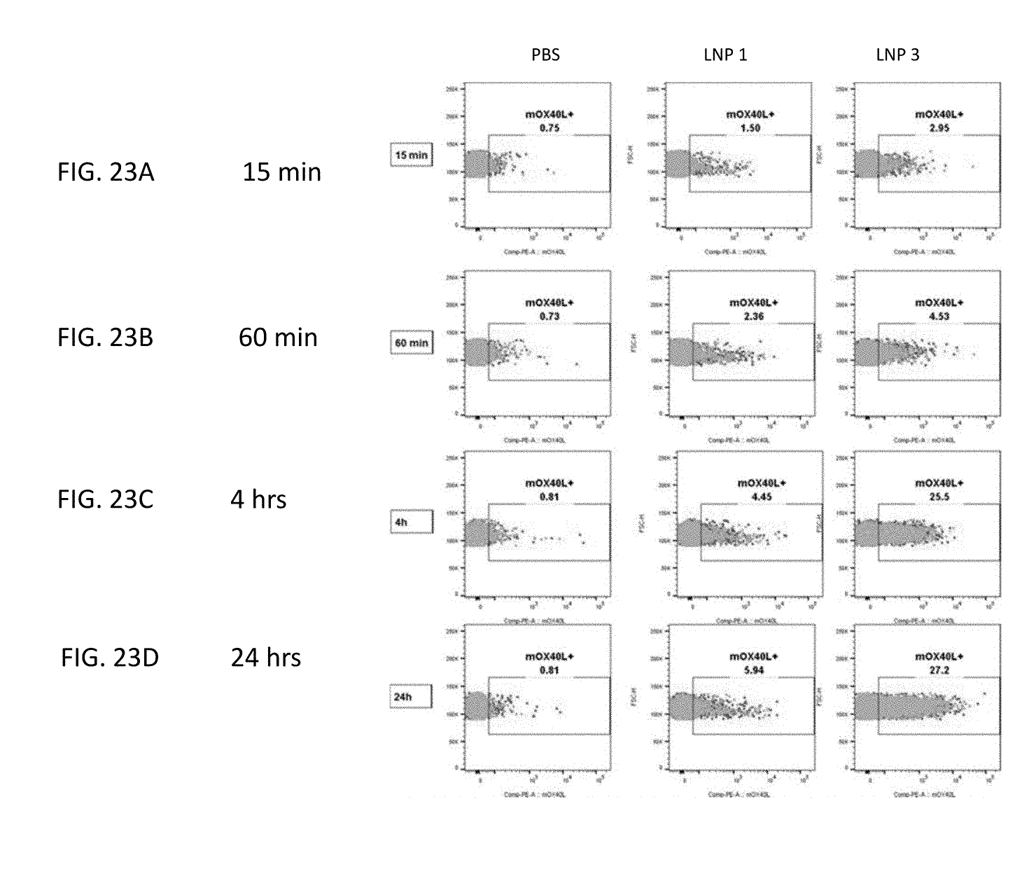

In this example, human PBMCs were transfected ex vivo for different amounts of time to determine the minimal LNP interaction time necessary to achieve transfection of human T cells. 2×10⁵ human PBMCs were incubated with 100 ng of LNP 3 encapsulating mOX40L-encoding mRNA for 15 minutes, 60 minutes, 4 hours or 24 hours. PBS and LNP 1 were used as controls. Flow cytometry results for mOX40L+ T cells at each time point are shown in FIGS. 23A-23D, respectively. The results demonstrate that 15 minutes incubation time was sufficient to transfect T cells, with levels of transfection increasing up to 4 hours. The levels of transfection observed at 4 hours and 24 hours were approximately equivalent. These results demonstrate that as little as 15 minutes of LNP interaction time is sufficient to transfect human T cells ex vivo.

### Example 14: Immune Response Enhancement Using Beta-Sitosterol/Cholesterol-Containing Lipid Nanoparticles

In this example, the ability of a beta-sitosterol/cholesterol-containing LNP to enhance immunogenicity of an mRNA vaccine in vivo, as compared to an LNP lacking beta-sitosterol/cholesterol, was examined. The vaccine composition was an mRNA encoding cytomegalovirus (CMV) glycoprotein B (gB) encapsulated in either an LNP containing Compound Y or an LNP containing both Compound Y and beta-sitosterol/cholesterol. Each of the two LNPs also contained DSPC, PEG-DMG and cholesterol.

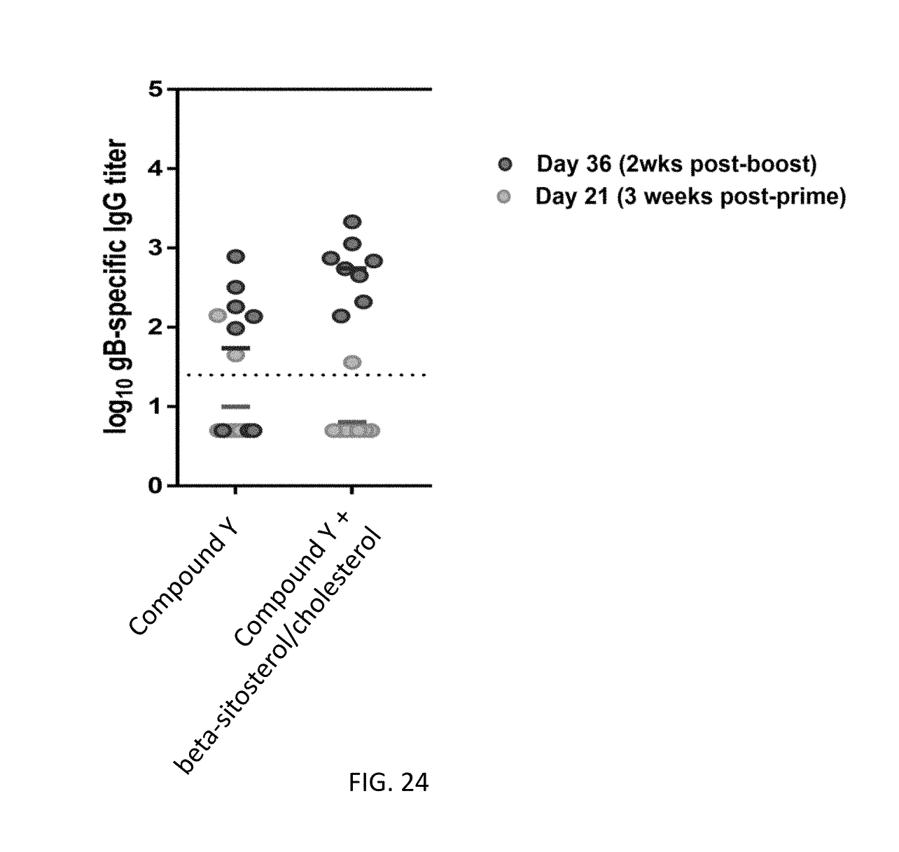

Mice were immunized intramuscularly with the mRNA vaccine (0.5 μg) encapsulated in each of the two LNPs on Day 1 and were boosted on Day 21. Serum gB-specific IgG titers were assayed by ELISA using gB coated plates on Day 21 (3 weeks post prime) and Day 36 (2 weeks post boost). The results are shown in FIG. 24. The results indicated that by Day 36 the anti-gB IgG response was greater in the mice treated with the beta-sitosterol/cholesterol-containing LNP as compared to the LNP lacking beta-sitosterol/cholesterol.

Thus, the results in FIG. 24 demonstrated that inclusion of beta-sitosterol/cholesterol in the LNP used in the mRNA vaccine enhanced the immune response in vivo to the antigen encoded by the mRNA vaccine, as compared to an LNP lacking beta-sitosterol/cholesterol.

### Example 15: mRNA Delivery in Mice In Vivo

In this example, the ability of the beta-sitosterol/cholesterol-containing LNP, as compared to the same LNP lacking beta-sitosterol/cholesterol, to deliver mRNA into murine splenic T cells, splenic B cells, splenic dendritic cells and bone marrow cells in vivo was tested.

Mouse OX40L-encoding mRNA was formulated into either Compound X/DSPC/PEG DMG (LNP 1) or Compound X/beta-sitosterol/cholesterol/DSPC/PEG DMG (LNP 3). C57B16/J mice were treated intravenously with 0.5 mg/kg mRNA encapsulated in either LNP 1 or LNP 2. PBS was used at a negative control. At twenty-four hours, spleen cells and bone marrow cells were harvested and analyzed for expression of mOX40L by flow cytometry.

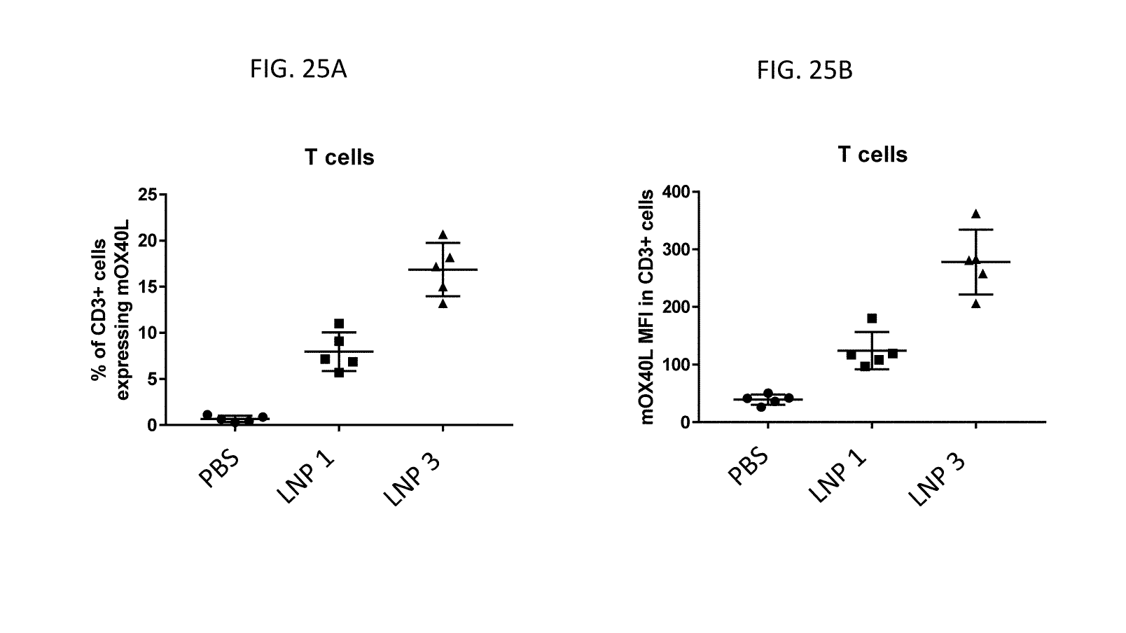

The flow cytometry results for mOX40L+ CD3+ splenic T cells are summarized in FIGS. 25A-25B, wherein FIG. 25A shows the percentage of CD3+ cells expressing mOX40L and FIG. 25B shows the mOX40L mean fluorescence index (MFI) in CD3+ cells. The results demonstrate that the beta-sitosterol/cholesterol-containing LNP delivered the mRNA construct to a higher percentage of splenic T cells, and resulted in a higher MFI in transfected T cells, as compared to the LNP lacking beta-sitosterol/cholesterol.

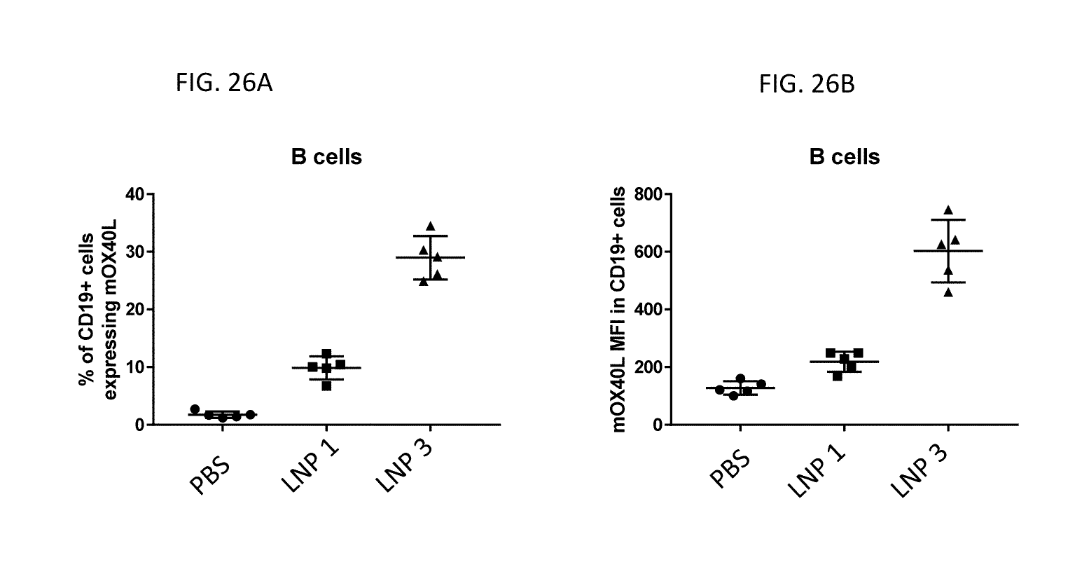

The flow cytometry results for mOX40L+ CD19+ splenic B cells are summarized in FIGS. 26A-26B, wherein FIG. 26A shows the percentage of CD19+ cells expressing mOX40L and FIG. 26B shows the mOX40L MFI in CD19+ cells. The results demonstrate that the beta-sitosterol/cholesterol-containing LNP delivered the mRNA construct to a higher percentage of splenic B cells, and resulted in a higher MFI in transfected B cells, as compared to the LNP lacking beta-sitosterol/cholesterol.

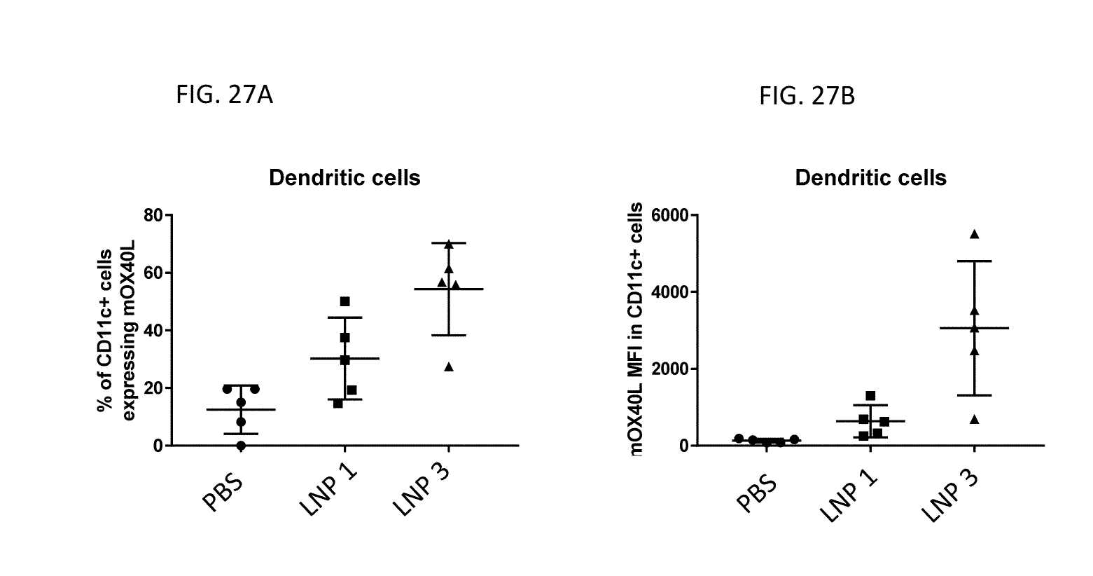

The flow cytometry results for mOX40L+ CD11c+ splenic dendritic cells are summarized in FIGS. 27A-27B, wherein FIG. 27A shows the percentage of CD11c+ cells expressing mOX40L and FIG. 27B shows the mOX40L MFI in CD11c+ cells. The results demonstrate that the beta-sitosterol/cholesterol-containing LNP delivered the mRNA construct to a higher percentage of splenic dendritic cells, and resulted in a higher MFI in transfected dendritic cells, as compared to the LNP lacking beta-sitosterol/cholesterol.

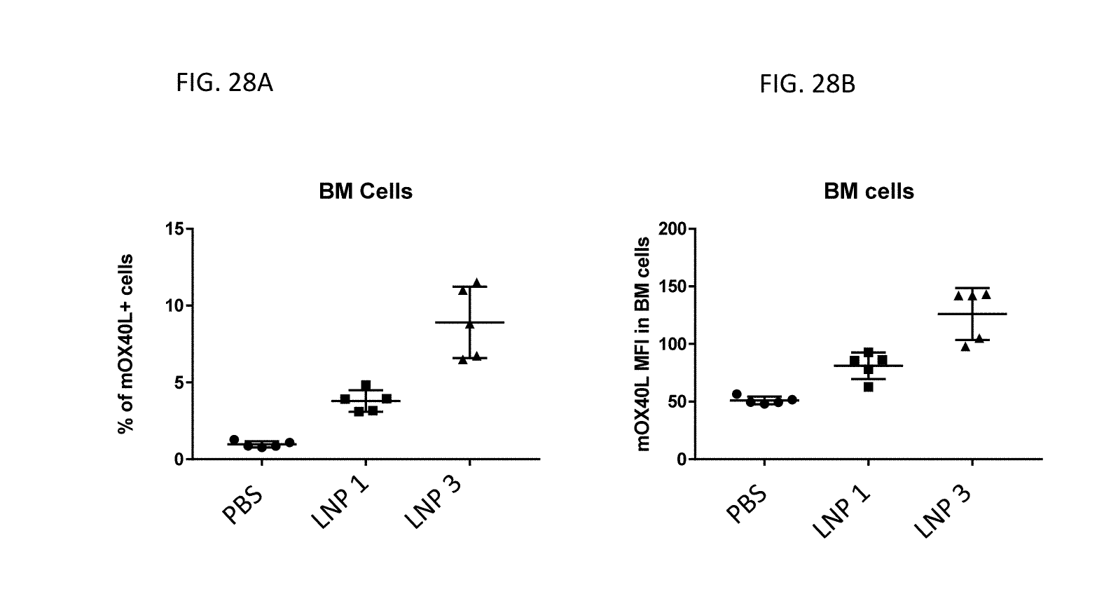

The flow cytometry results for mOX40L+ bone marrow cells are summarized in FIGS. 28A-28B, wherein FIG. 28A shows the percentage of bone marrow cells expressing mOX40L and FIG. 28B shows the mOX40L MFI in bone marrow cells. The results demonstrate that the beta-sitosterol/cholesterol-containing LNP delivered the mRNA construct to a higher percentage of bone marrow cells, and resulted in a higher MFI in transfected bone marrow cells, as compared to the LNP lacking beta-sitosterol/cholesterol.

Repeat dosing in mice in vivo confirmed expression of the mRNA construct in murine splenic T cells, splenic B cells, splenic dendritic cells and bone marrow cells, thereby confirming transfection of multiple immune cell types in mice in vivo.

Accordingly, these studies demonstrated that the beta-sitosterol/cholesterol-containing LNP effectively delivered a protein-encoding mRNA construct in vivo into murine splenic T cells, splenic B cells, splenic dendritic cells and bone marrow cells resulting in significantly greater percentage of cells transfected and higher levels of protein expression all cell types examined, as compared to the same LNP lacking beta-sitosterol/cholesterol. Interestingly, this is in contrast to the lack of transfection observed for murine T cells when mouse PBMCs were incubated ex vivo with the beta-sitosterol/cholesterol-containing LNP, as demonstrated in Example 5 and FIG. 7.

### Example 16: Induction of Cytotoxicity Induced by mRNA Encoding a Modified Membrane Protein, CAR, in Transfected T Cells

In this example, the beta-sitosterol/cholesterol-containing LNP (LNP 3) was used to transfect T cells ex vivo with an anti-CD33 CAR T mRNA construct and the transfected T cells were tested for their ability to kill CD33+ AML cells in vitro.

Human PBMCs were transfected ex vivo with an anti-CD33 CAR T mRNA construct encapsulated in LNP 3 and incubated overnight. A CD34 mRNA construct was used as a control. Following incubation, T cells were isolated using EasySep™ Human T Cell Isolation kit (StemCell Technologies). Isolated T cells were then plated with CD33+ AML cells at ratios of 1:1 or 1:5 (with a constant number of AML cells at 1×10⁵ cells/well). The cytotoxicity dye Incucyte was included in the culture at 250 nM to assess cell killing. Cells were incubated with the Incucyte dye and green fluorescence (indicating cell death) was read at 2 hour intervals. Cells were treated with Triton 100 before the last reading to determine maximal cytotoxicity. Cytotoxicity index (CI) was then calculated according to: CI=(cytotoxicity at x hours/cytotoxicity maximal)×100.

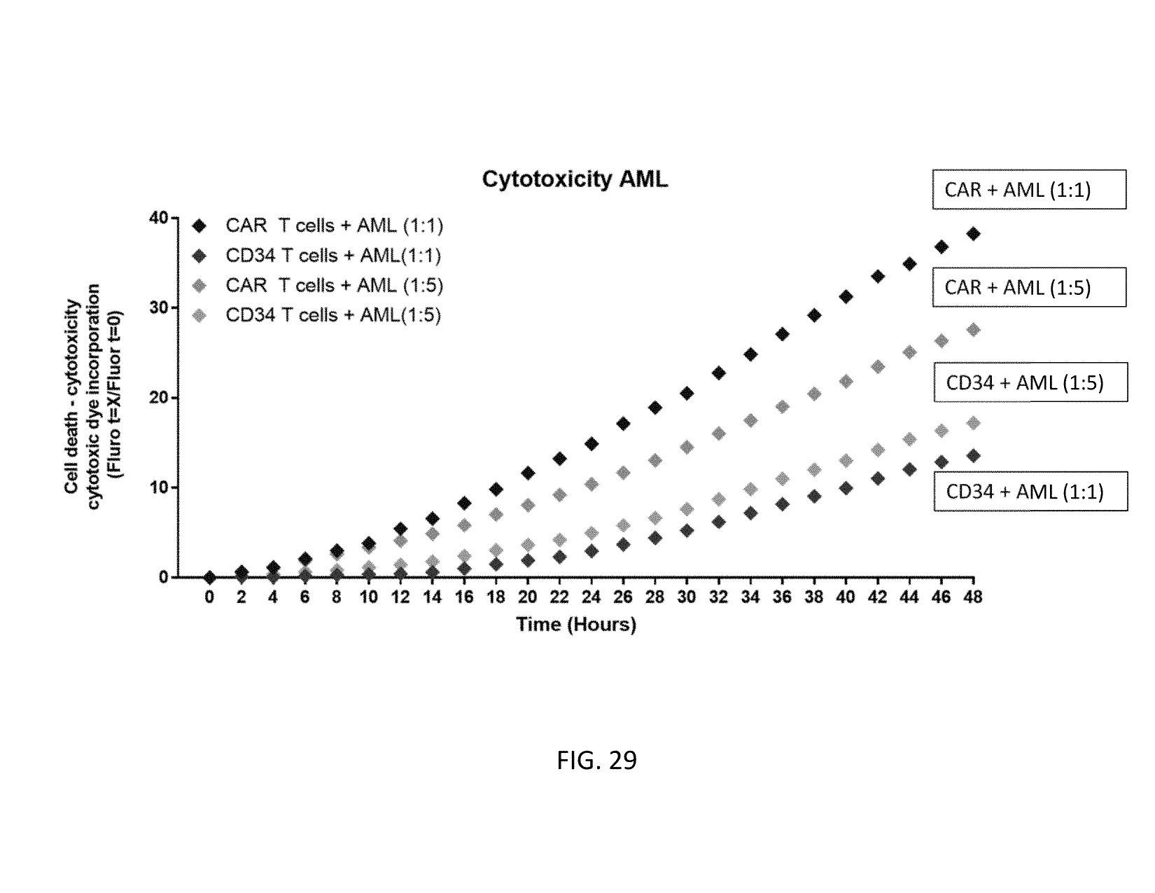

The results are shown in FIG. 29, which demonstrates that the T cells transfected with the anti-CD33 CAR T mRNA using LNP 3 were effective in killing CD33+ AML cells in vitro.

### Example 17: Immune Cell Delivery Potentiating Lipids for Transfection of T Cells

In this Example, LNPs were made with various lipid components and at different ratios and were tested for their ability to transfect T cells in vitro. Transfection was performed as in Example 5 using LNPs encapsulating an mRNA encoding mouse OX40L.

In a first set of experiments, phytosterol was used as the immune cell delivery potentiating lipid and other lipid constituents of the LNP were varied. As shown in Table 23 below, enhanced delivery to T cells was observed with either Compound X or Compound Y as the ionizable lipid, as evidence by the percent of cells positive for murine OX40L. In addition, enhanced delivery to T cells was observed using either PEG DMG or Compound 428 as the PEG lipid. Enhanced delivery to T cells was observed when the mol percent of phytosterol was either 18.5% or 28.5% (the total mol % of structural lipid was held constant at 38.5%.

TABLE 23
mOX40L EXPRESSION IN CD3+ T CELLS

| | | Structural lipid | | % Positive cells | |
|---|---|---|---|---|---|
| Ionizable lipid | Phospholipid | (cholesterol/phytosterol) | PEG Lipid | Buffer 1 | Buffer 2 |
| 50 mol % | 10 mol % | Total mol % 38.5 | 1.5 mol % | (Tris) | (PBS) |
| Cmpd X | DSPC | 0% β-sitosterol | PEG DMG | 0.13 | 0.16 |
| | | | Cmpd P 428 | 0.33 | 0.35 |
| | | 18.5% β-sitosterol | PEG DMG | 46.0 | 51.8 |
| | | | Cmpd P 428 | 36.9 | 43.0 |
| | | 28.5% β-sitosterol | PEG DMG | 50.1 | 53.3 |
| | | | Cmpd P 428 | 44.4 | 43.4 |
| Cmpd Y | DSPC | 0% β-sitosterol | PEG DMG | 0.97 | 1.12 |
| | | | Cmpd P 428 | 0.6 | 0.48 |
| | | 18.5% β-sitosterol | PEG DMG | 21.6 | 17.9 |
| | | | Cmpd P 428 | 14.9 | 4.30 |
| | | 28.5% β-sitosterol | PEG DMG | 45.5 | 13.2 |
| | | | Cmpd P 428 | 14.0 | 6.64 |

Results for individual samples with varying ratios of the LNP components are shown below in Table 24 (for Tris buffer) and in Table 25 (for PBS buffer).

TABLE 24
T cell Transfection in Buffer 1 (Tris) with Varying Ratios
of LNP Components

| | | | | | | Percent |
|---|---|---|---|---|---|---|
| | % | % | | % β- | % | mOX40L |
| Sam- | Ionizable | Phospho | % | sito- | Cmpd | + |
| ple | Cmpd X | DSPC | Cholesterol | sterol | 428 | T cells |
| 43 | 50 | 10 | 38.5 | 0 | 1.5 | 0.53 |
| 44 | 50 | 10 | 20 | 18.5 | 1.5 | 39.1 |
| 45 | 50 | 10 | 10 | 28.5 | 1.5 | 8.96 |
| 46 | 50 | 10 | 0 | 38.5 | 1.5 | 3.20 |
| 47 | 60 | 0 | 10 | 28.5 | 1.5 | 0.98 |
| 48 | 60 | 0 | 20 | 18.5 | 1.5 | 0.52 |
| 49 | 40 | 20 | 10 | 28.5 | 1.5 | 38.1 |
| 50 | 40 | 20 | 20 | 18.5 | 1.5 | 60.7 |
| 51 | 30 | 30 | 10 | 28.5 | 1.5 | 1.33 |
| 52 | 30 | 30 | 20 | 18.5 | 1.5 | 2.47 |
| 53 | 50 | 20 | 10 | 18.5 | 1.5 | 51.2 |
| 54 | 50 | 20 | 0 | 28.5 | 1.5 | 29.5 |
| 55 | 50 | 30 | 0 | 18.5 | 1.5 | 26.0 |
| 56 | 50 | 30 | 18.5 | 0 | 1.5 | 45.6 |

TABLE 25
T Cell Transfection in Buffer 2 (PBS) with Varying
Ratios of LNP Components

| | | | | | | Percent |
|---|---|---|---|---|---|---|
| | % | % | | % | % | mOX40L |
| Sam- | Ionizable | Phospho | % | β-sito- | Cmpd | + |
| ple | Cmpd X | DSPC | Cholesterol | sterol | 428 | T cells |
| 29 | 50 | 10 | 38.5 | 0 | 1.5 | 1.70 |
| 30 | 50 | 10 | 20 | 18.5 | 1.5 | 52.7 |
| 31 | 50 | 10 | 10 | 28.5 | 1.5 | 53.7 |
| 32 | 50 | 10 | 0 | 38.5 | 1.5 | 15.3 |
| 33 | 60 | 0 | 10 | 28.5 | 1.5 | 0.28 |
| 34 | 60 | 0 | 20 | 18.5 | 1.5 | 0.51 |
| 35 | 40 | 20 | 10 | 28.5 | 1.5 | 65.8 |
| 36 | 40 | 20 | 20 | 18.5 | 1.5 | 73.3 |
| 37 | 30 | 30 | 10 | 28.5 | 1.5 | 0.82 |
| 38 | 30 | 30 | 20 | 18.5 | 1.5 | 0.16 |
| 39 | 50 | 20 | 10 | 18.5 | 1.5 | 66.9 |
| 40 | 50 | 20 | 0 | 28.5 | 1.5 | 52.3 |
| 41 | 50 | 30 | 0 | 18.5 | 1.5 | 50.9 |
| 42 | 50 | 30 | 18.5 | 0 | 1.5 | 12.5 |

In a second series of experiments, additional compounds, were tested in the LNPs for their ability to enhance delivery to T cells. For these experiments, the association of the LNP with cells and the expression of mRNA-encoding protein within the transfected cells was assessed at the cell-by-cell level. Peripheral blood mononuclear cells (PBMCs) from human donors were used. PBMCs were predominately T cells (40-60%), B cells (3-15%), and monocytes (15-35%), but also contained small numbers of other cell types. LNPs were incubated with serum or PBS for 30 minutes and then added to the PBMCs. The LNP concentration was 0.050 mg/ml, the LNP volume was 0.1 ml.

LNPs contained 0.1% Rhodamine-DOPE in addition to the other LNP components to facilitate detection. Thus, the ratios set forth in the tables below include 0.1 for the rhodamine label. For example, 50:10:10:27.9:2:0.1 AL:DSPC:chol:sterol:Cmp 428 indicates 50% amino lipid as the ionizable lipid, 10% DSPC as the phospholipid, 10% cholesterol, 27.9% phytosterol, 2% Compound 428 as the PEG lipid and 0.1% for the rhodamine-DOPE label. To prepare a non-labeled LNP, the same ratios are used except that the 0.1% rhodamine label is omitted and an additional 0.1% of structural lipid (cholesterol or cholesterol/phytosterol blend) is used (e.g., 27.9% becomes 28%).

Cells were sorted by flow cytometry into subsets based on expression of certain markers: CD3 (T cells), CD20 (B cells), and CD3−, CD20−, CD14+ (Monocytes). mRNA encoding GFP was used as a model agent to effect expression of an intracellular protein.

When conducting flow cytometry, the Y-axis was signal from the rhodamine tag on the LNP and the X-axis was the GFP signal showing expression of the protein.

The “associated” population is any rhodamine+ and/or GFP+ cell and the percent of these cells was calculated by dividing by the total number of cells. The “expression” population is any GFP+ cell and the percent of these cells was calculated by dividing by the total number of cells. The percent of associated cells expressing was obtained by dividing the % expressing cells by the % associated cells.

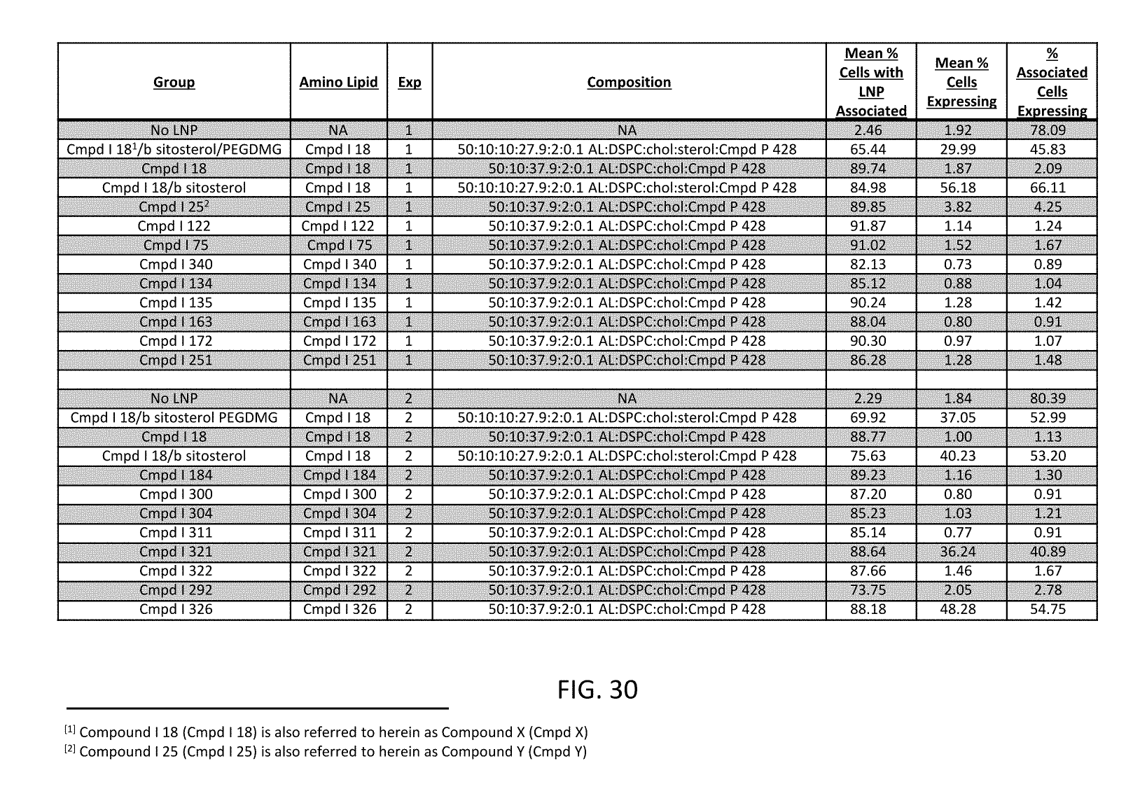

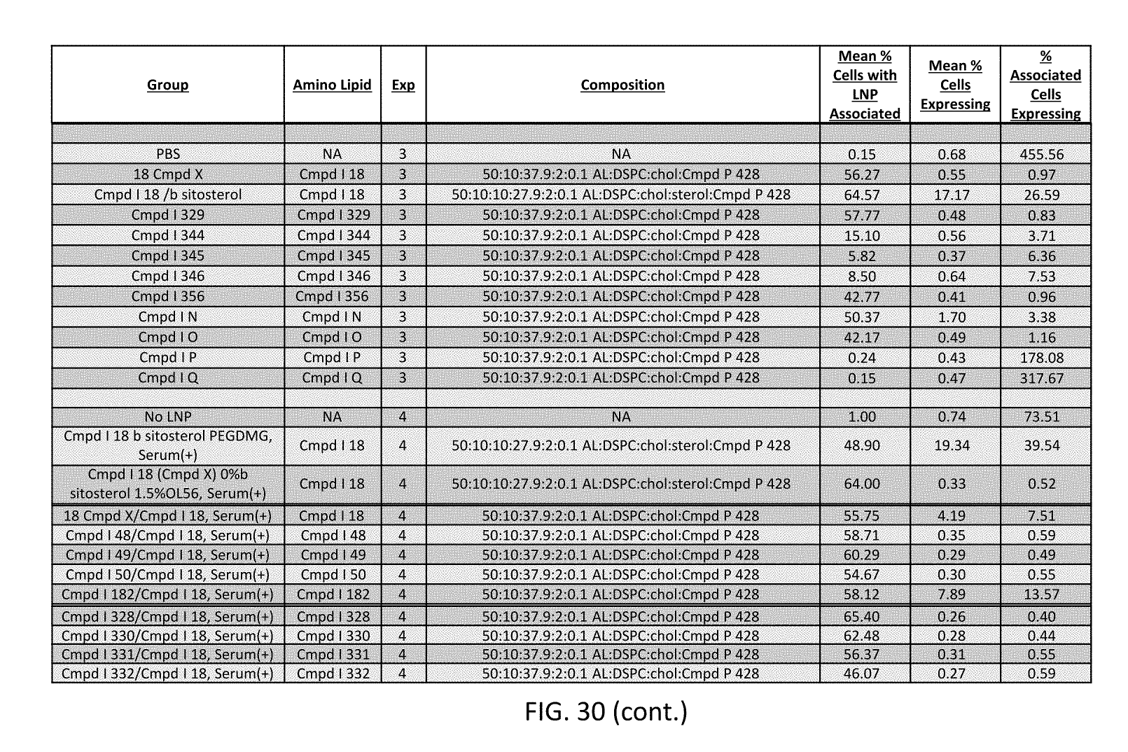

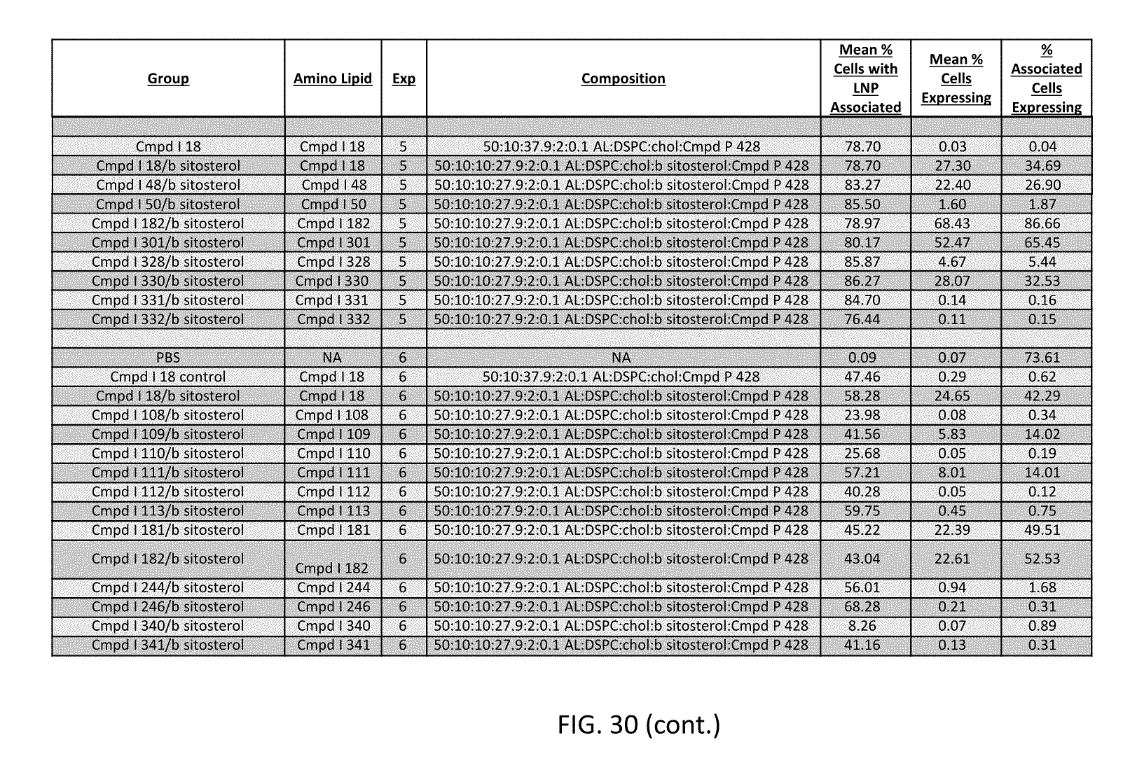

The results for a panel of amino lipids tested for the ionizable lipid component of the LNP are shown in FIG. 30. The results in FIG. 30 demonstrated that in particular amino lipid Compound X, Compound Y, Compound I-321, Compound I-292, Compound I-326, Compound I-182, Compound I-301, Compound I-48, Compound I-50, Compound I-328, Compound I-330, Compound I-109, Compound I-111, and Compound I-181 exhibited immune cell delivery potentiating ability in T cells. In addition, as shown in Experiment 2, LNPs with either Compound I-321 or I-326 as the ionizable lipid with only cholesterol as the structural lipid (and no other sterol) were effective at potentiating delivery and uptake by T cells as shown by the mean % cells expressing the mRNA relative to the positive control of an LNP with Compound X (Compound I-18) as the ionizable lipid and β-sitosterol as the structural lipid.

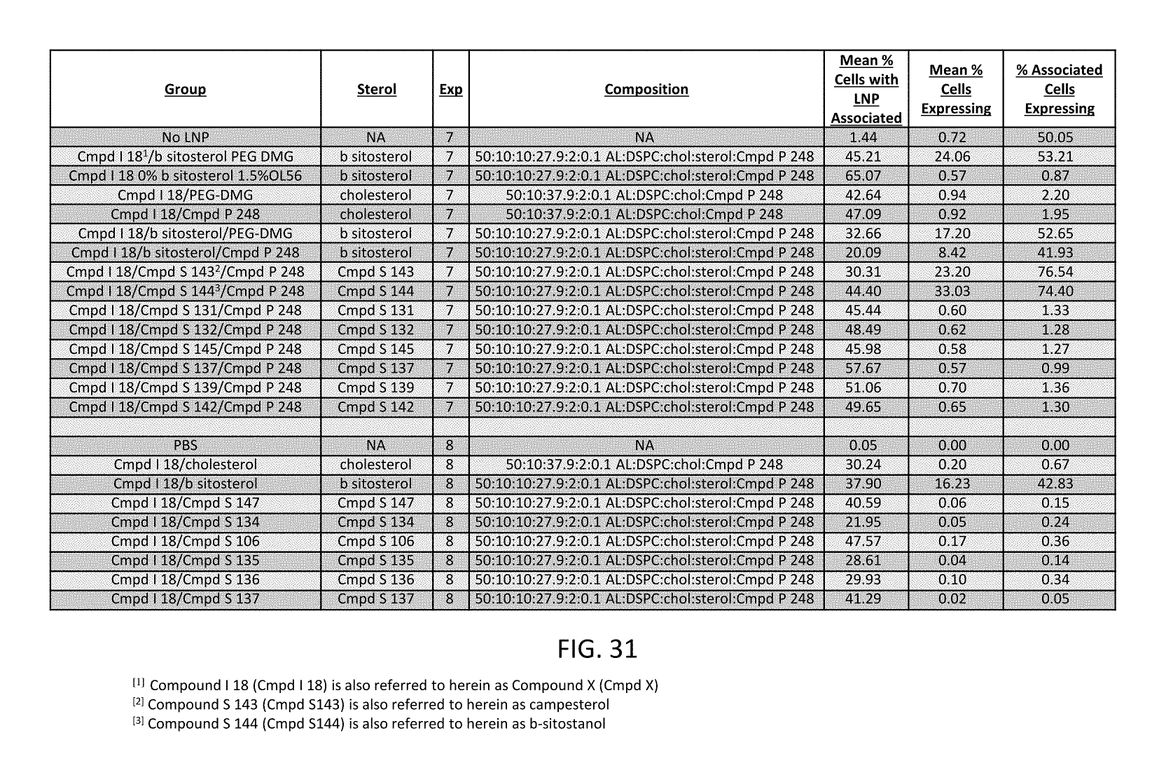

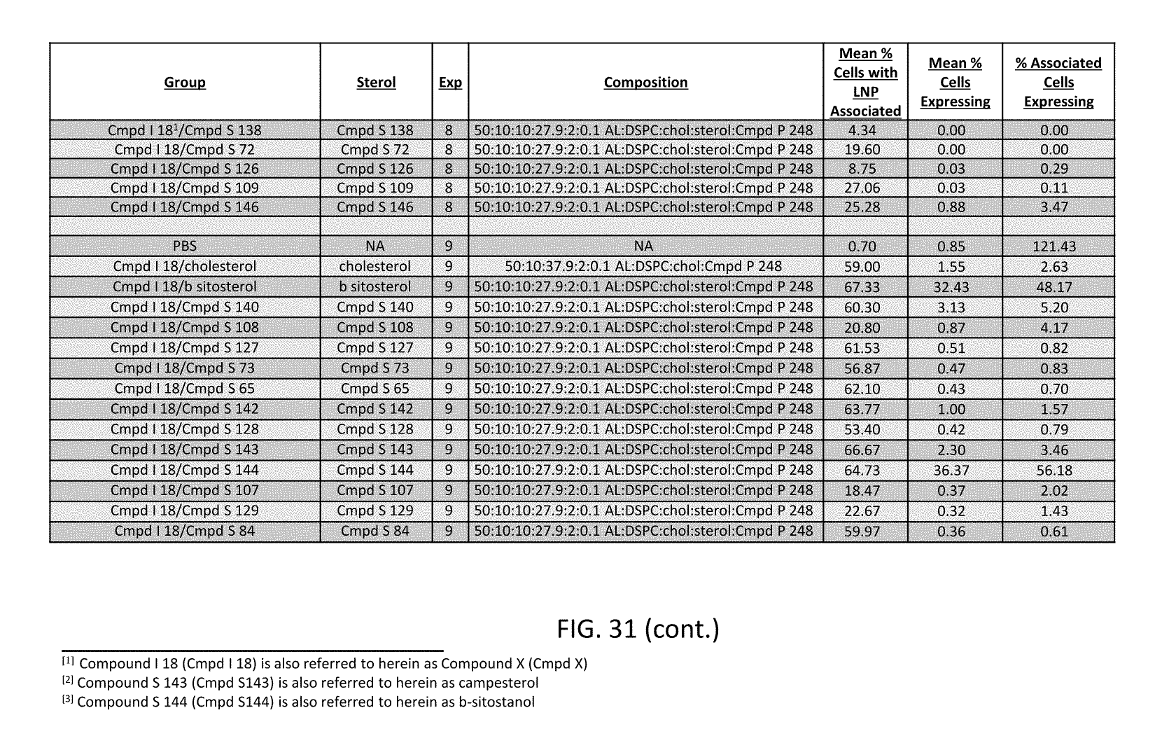

The results for a panel of sterols tested for the structural lipid component of the LNP are shown in FIG. 31. The results in FIG. 31 demonstrated that, in addition to β-sitosterol, in particular sterol compounds camposterol and β-sitostanol exhibited immune cell delivery potentiating ability in T cells.

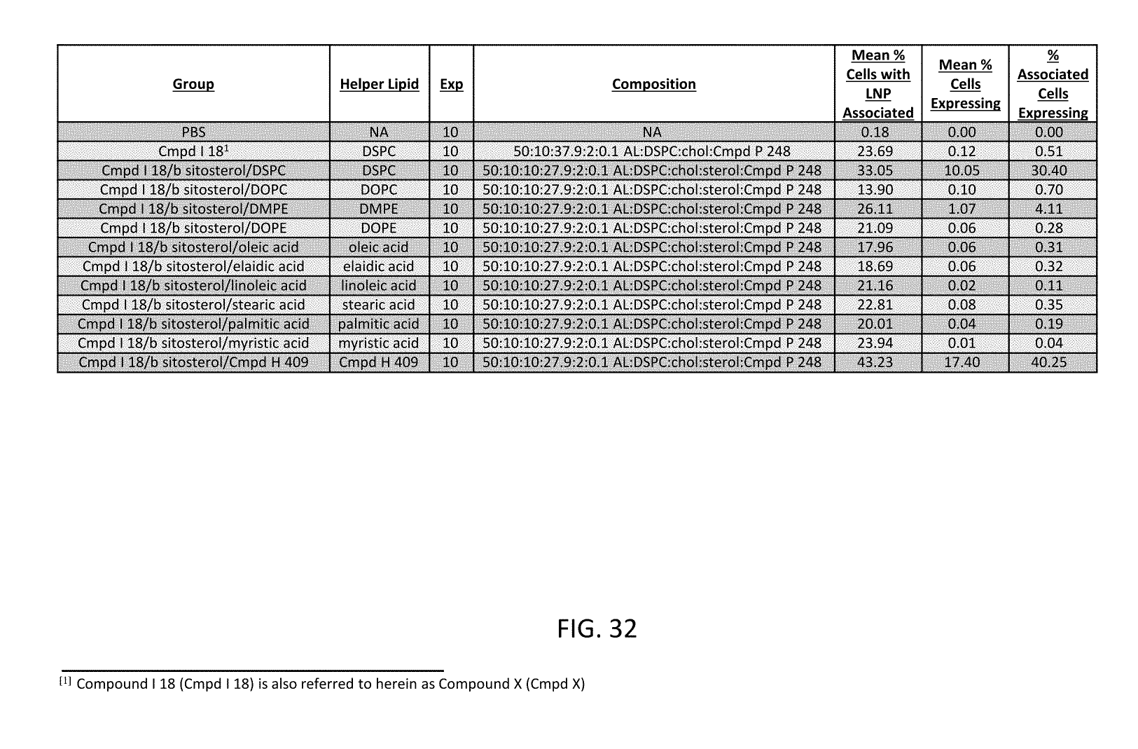

The results for a panel of helper lipids tested for the phospholipid component of the LNP are shown in FIG. 32. The results in FIG. 32 demonstrated that in particular phospholipid Compounds H-409, DSPC and DMPE exhibited immune cell delivery potentiating ability in T cells.

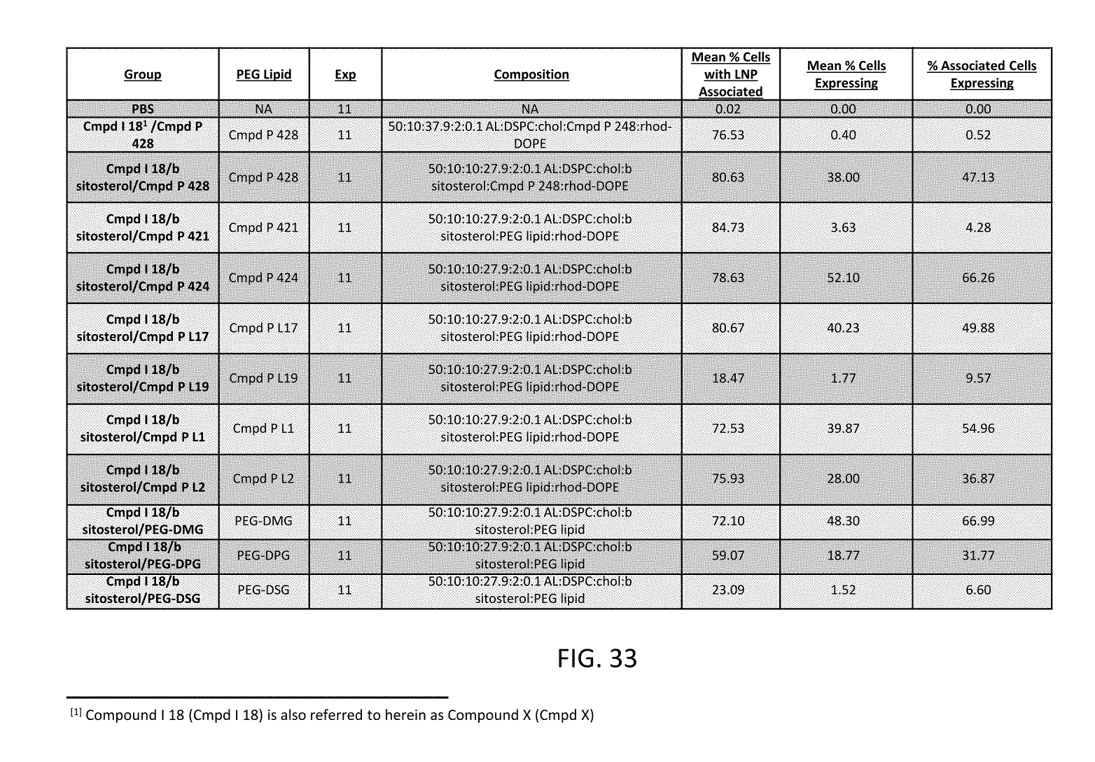

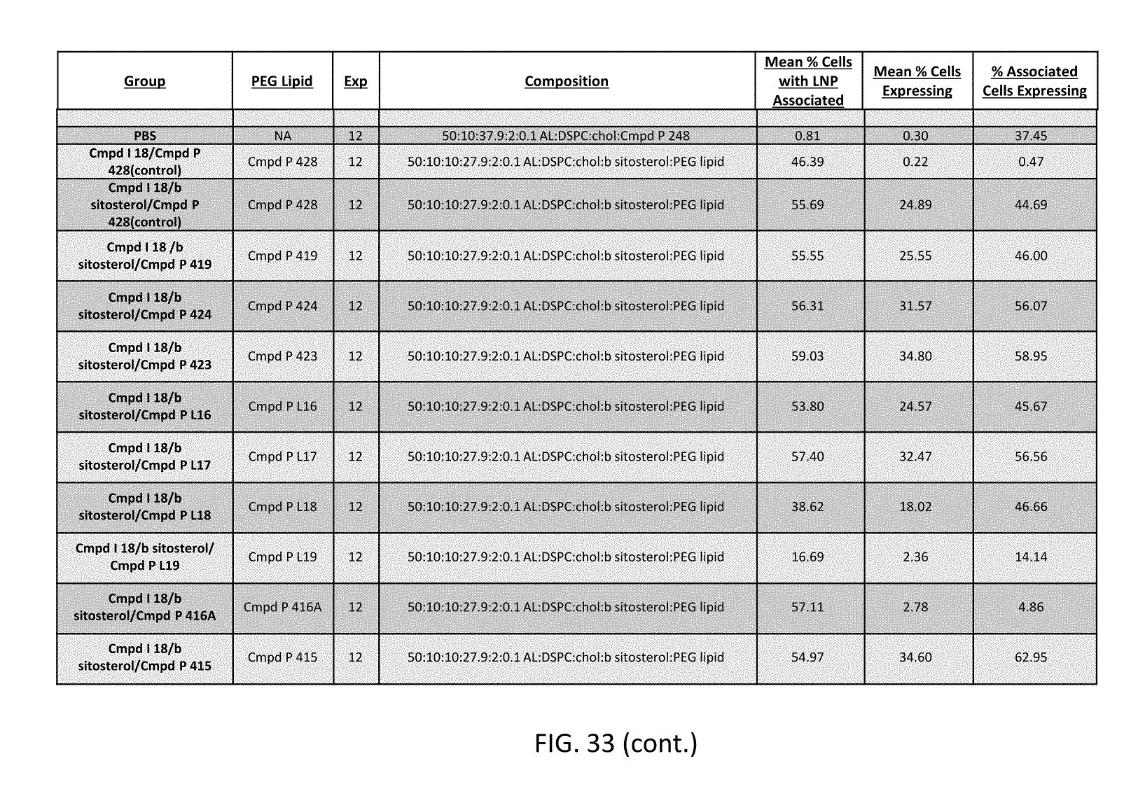

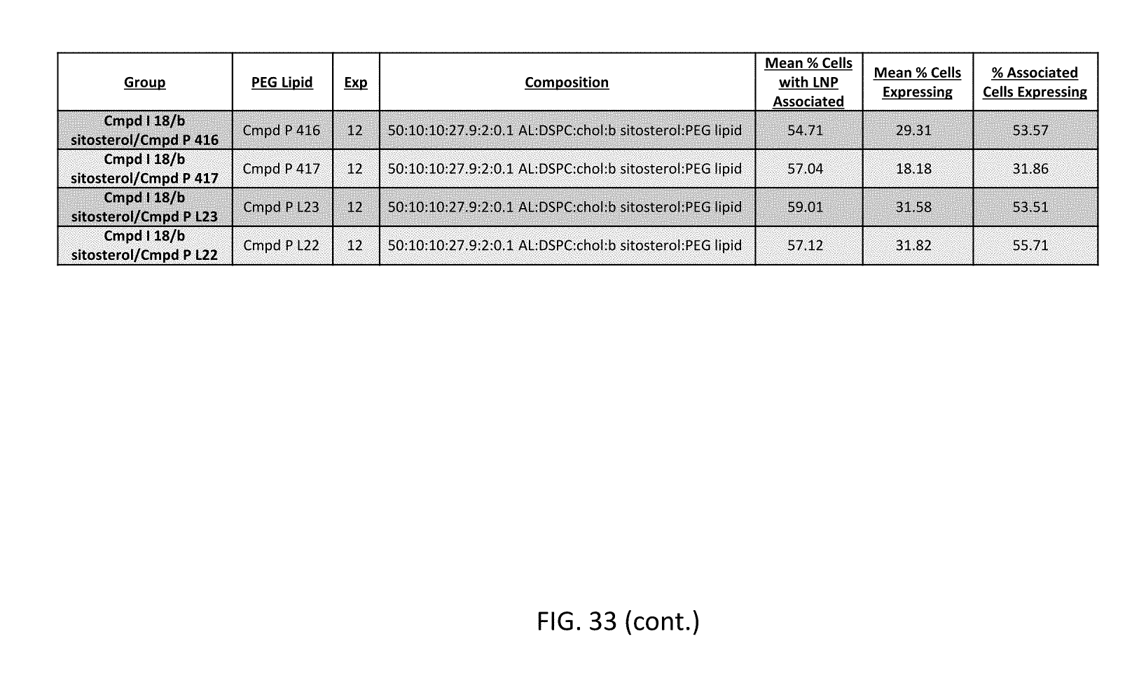

The results for a panel of PEG compounds tested for the PEG lipid component of the LNP are shown in FIG. 33. The results of the PEG lipid screening demonstrated that in particular PEG lipid Compounds P415, P416, P417, P 419, P 420, P 423, P 424, P 428, P L1, P L2, P L16, P L17, P L18, P L19, P L22 and P L23, DMG, DPG, and DSG exhibited immune cell delivery potentiating ability in T cells.

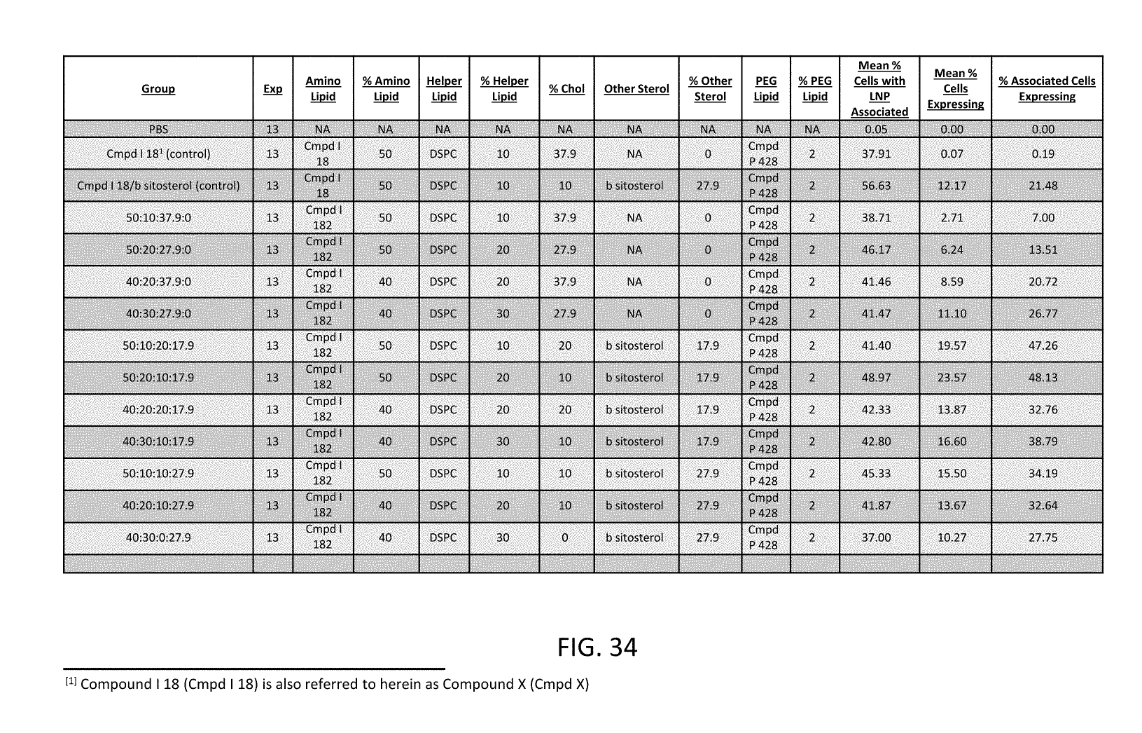

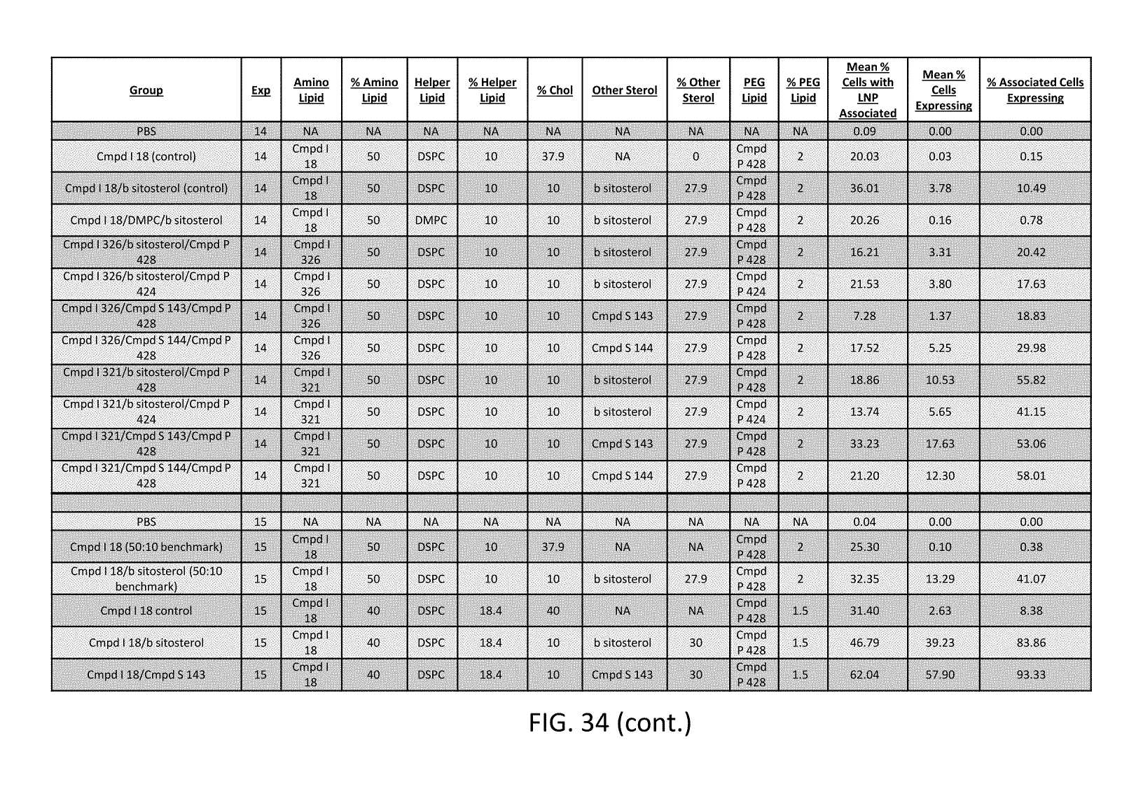

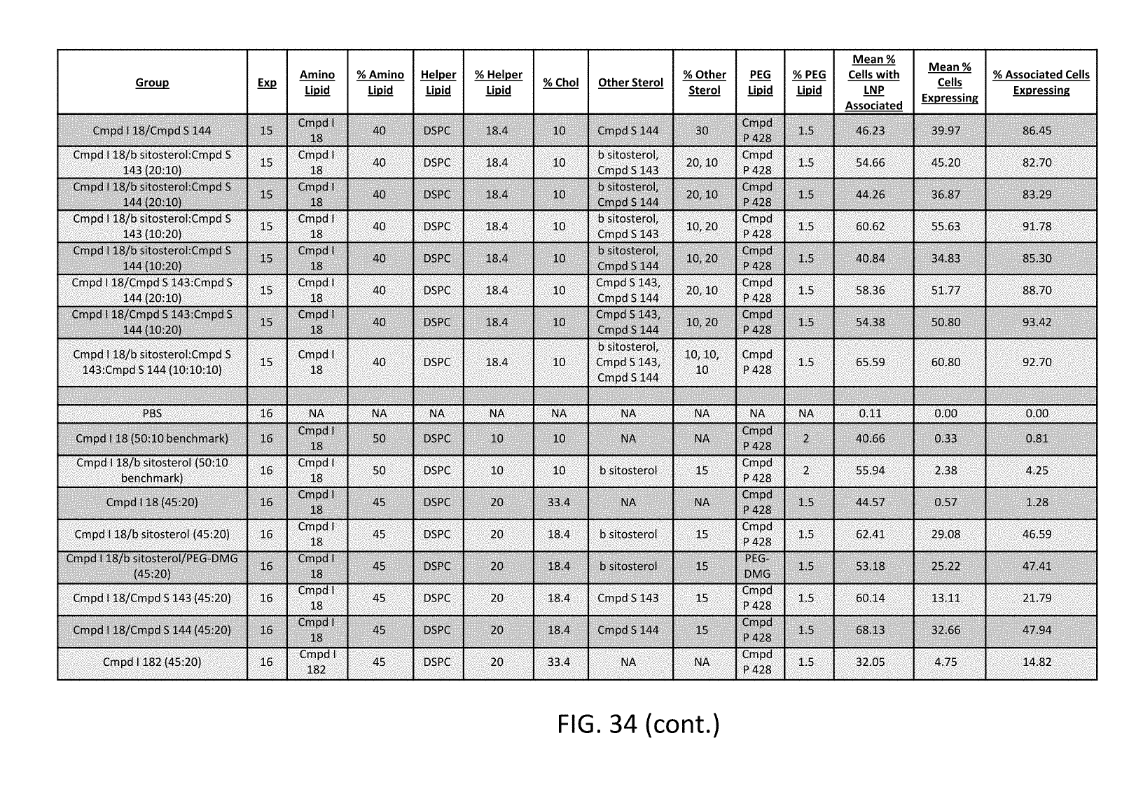

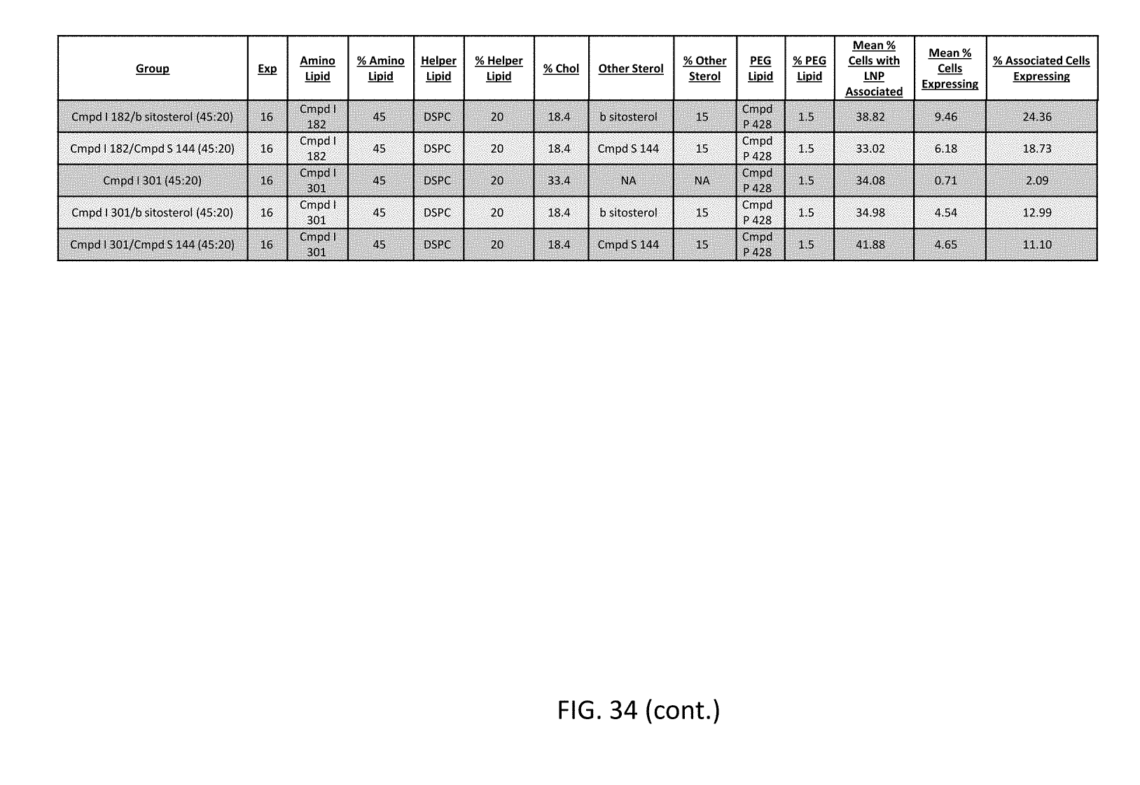

The results for a panel of compositions formulated using various compounds identified in the above screens as component of the LNP are shown in FIG. 34.

The formulations tested in Experiment 13 as shown in FIG. 34 used Compound I-182 as the amino lipid, DSPC as the helper lipid, cholesterol/β-sitosterol as the structural lipid and Compound P-428 as the PEG Lipid, with ratios varied as indicated in FIG. 34. Controls were LNPs using Compound I-18 as the amino lipid and either cholesterol alone (negative control) or cholesterol/β-sitosterol (positive control) as the structural lipid. The results for Experiment 13 demonstrated that all of the Compound I-182-containing LNPs exhibited higher mean % cells expressing the mRNA and % of associated cells expressing the mRNA as compared to the negative controls. Moreover, many of the Compound I-182-containing LNPs exhibited higher LNP association and/or % cells expressing mRNA than the Compound I-18-containing positive control. In particular, LNP formulations containing Compound I-182/DSPC/cholesterol/β-sitosterol/P 428 at ratios of 50:10:20:17.9:2 or 50:20:10:17.9:2 exhibited significantly higher mean % cells expressing the mRNA and % of associated cells expressing the mRNA than the positive control.

The formulations tested in Experiment 14 as shown in FIG. 34 used Compound I-326 or I-321 as the amino lipid, DSPC as the helper lipid, cholesterol/β-sitosterol or Compound S-143 or S-144 as the structural lipid and Compound P-428 or P 424 as the PEG Lipid. Controls were LNPs using Compound I-18 as the amino lipid and either cholesterol alone (negative control) or cholesterol/β-sitosterol (positive control) as the structural lipid. The results for Experiment 14 demonstrated that all of the formulations tested exhibited approximately equal or higher mean % cells expressing the mRNA and/or % of associated cells expressing the mRNA than the positive control. In particular, the formulation containing Compound I-321/DSPC/cholesterol/β-sitosterol or Compound S-143 or Compound S-144/P 428 exhibited significantly higher mean % cells expressing the mRNA and % of associated cells expressing the mRNA than the positive control.

The formulations tested in Experiment 15 as shown in FIG. 34 used Compound I-18 as the amino lipid, DSPC as the helper lipid, cholesterol/β-sitosterol and/or Compound S-143 and/or Compound S-144 as the structural lipid and Compound P-428 as the PEG Lipid. Controls were LNPs using Compound I-18 as the amino lipid and either cholesterol alone (negative control) or cholesterol/β-sitosterol (positive control) as the structural lipid. The results for Experiment 15 demonstrated that all of the formulations tested exhibited higher mean % cells expressing the mRNA and/or % of associated cells expressing the mRNA than the positive control. In particular, formulations containing Compound I-18/DSPC/cholesterol/β-sitosterol+ either Compound S-143, Compound S-144 or both Compound S-143 and Compound S-144/P 428 exhibited significantly higher mean % cells expressing the mRNA and % of associated cells expressing the mRNA than the positive control.

The formulations tested in Experiment 16 as shown in FIG. 34 used Compound I-18 or Compound I-182 or Compound I-301 as the amino lipid, DSPC as the helper lipid, cholesterol/β-sitosterol and/or Compound S-143 and/or Compound S-144 as the structural lipid and Compound P-428 or PEG-DMG as the PEG Lipid. Controls were LNPs using Compound I-18 or Compound I-182 or Compound I-301 as the amino lipid and either cholesterol alone (negative control) or cholesterol/β-sitosterol (positive control) as the structural lipid. The results for Experiment 16 demonstrated that all of the formulations tested exhibited approximately equal or higher mean % cells expressing the mRNA and/or % of associated cells expressing the mRNA than the relevant positive control.

The results in FIG. 34 also showed that changes in the ratios of the components of the LNP (e.g., a reduction in the mol % of amino lipid from 50 mol % to 40 or 45 mol % amino lipid or increase in the mol % of helper lipid from 10% to 18.4 or 20 mol %) did not negatively impact the immune potentiating delivery of the LNPs to T cells.

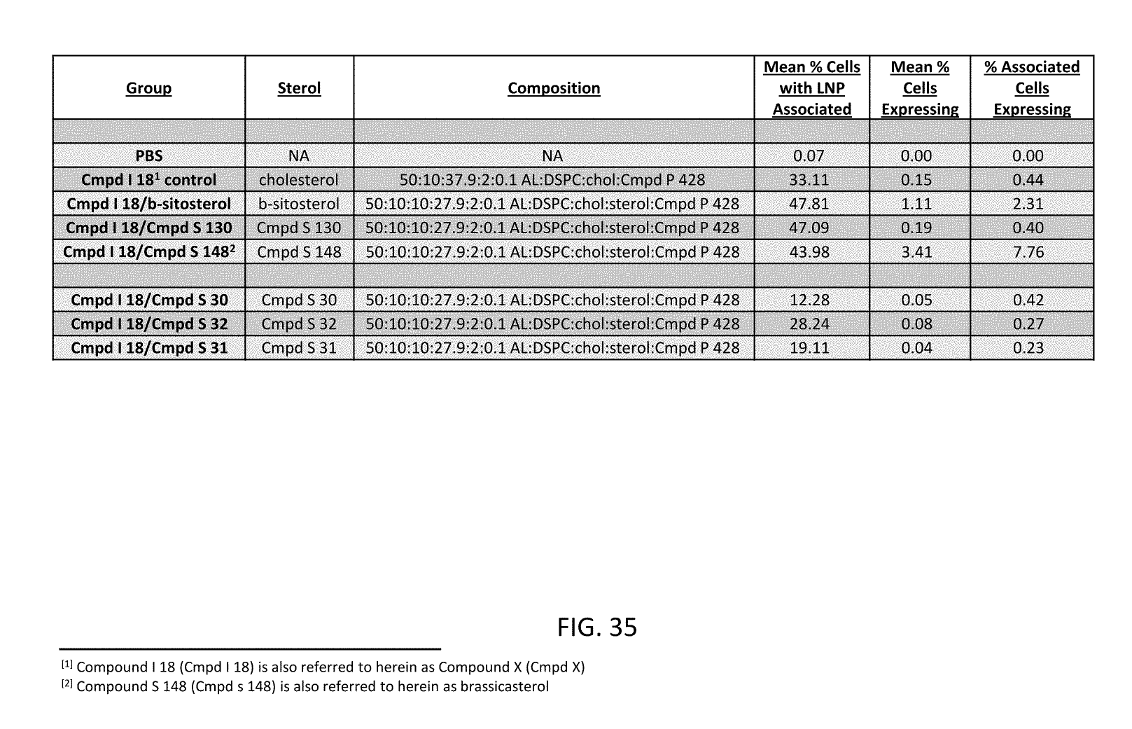

In a third series of experiments, an additional panel of sterol compounds was tested in LNPs for T cell delivery in vitro. The results for this panel of sterols tested for the structural lipid component of the LNP are shown in FIG. 35. The results in FIG. 35 demonstrated that, in particular, the phytosterol brassicasterol (Compound S-148) exhibited immune cell delivery potentiating ability in T cells.

### Example 18: Transfection of T Cells In Vivo Using Immune Cell Delivery Potentiating Lipids

In this Example, LNPs were made with various lipid components identified in the in vitro screens described in Example 17 and mice were administered the LNPs encapsulating a murine OX40L-encoding mRNA. The compositions shown below in Table 26 were tested in which the ratios of the components in each composition was 50% ionizable lipid, 10% phospholipid, 38.5% structural lipid and 1.5% PEG lipid.

TABLE 26
Compositions Tested In vivo

| | Ionizable | | Structural | |
|---|---|---|---|---|
| Group | Lipid | Phospholipid | Lipid | PEG Lipid |
| 1 | Compound X | DSPC | β-sitosterol | Cmpd P 428 |
| 2 | Compound X | DSPC | camposterol | Cmpd P 428 |
| 3 | Compound X | DSPC | β-sitostanol | Cmpd P 428 |
| 4 | Compound I-182 | DSPC | β-sitosterol | Cmpd P 428 |
| 5 | Compound I-301 | DSPC | β-sitosterol | Cmpd P 428 |
| 6 | Compound I-321 | DSPC | cholesterol | Cmpd P 428 |
| 7 | Compound I-321 | DSPC | β-sitosterol | Cmpd P 428 |
| 8 | Compound I-326 | DSPC | cholesterol | Cmpd P 428 |
| 9 | Compound I-326 | DSPC | β-sitosterol | Cmpd P 428 |

Mice were treated intravenously with a single injection at a dose concentration of 0.1 mg/ml and at a dose level of 0.5 mg/kg. PBS was used as a negative control. Compound X (Compound I-18), which previously had been demonstrated as an immune cell delivery potentiating lipid, was used as a positive control. Levels of T cell transfection in the spleen at 24 hours post injection were determined by the percentage of CD3+ mOX40L+ cells in the splenic cell population.

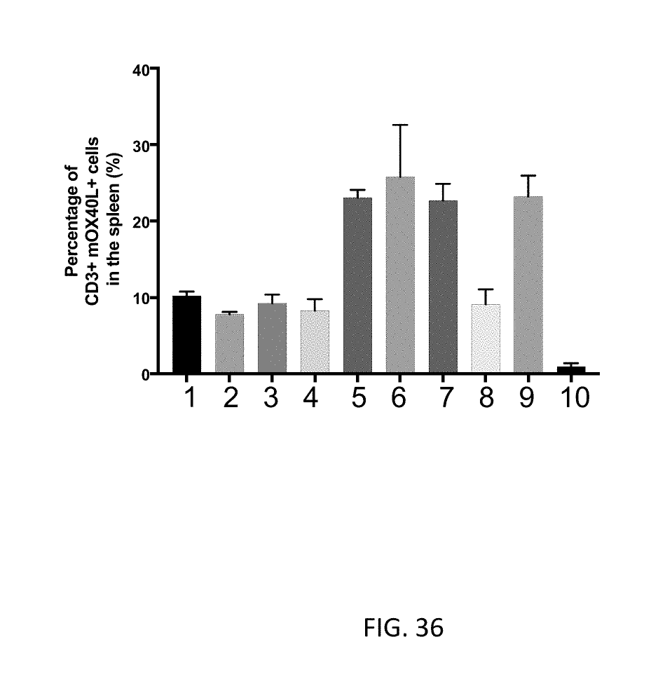

The results are shown in FIG. 36, wherein the bars/lanes labeled 1-9 correspond to Groups 1-9, respectively, shown above, and bar labeled 10 corresponds to the PBS control. The results demonstrate that LNPs containing either Compound I-301, Compound I-321 or Compound I-326 as the ionizable lipid component all demonstrated an even higher capability to transfect splenic CD3+ T cells in vivo than the positive control LNP containing Compound X. In addition, as shown in FIG. 36, lanes 6 and 8, LNPs containing either Compound I-321 or I-326 as the ionizable lipid with only cholesterol as the structural lipid (and no other sterol) were effective at potentiating transfecting CD3+ T cells in vivo relative to the positive control LNP with Compound X (Compound I-18) as the ionizable lipid and β-sitosterol as the structural lipid.

### Example 19: Immune Cell Delivery Potentiating Lipids for Transfection of Monocytes

In this Example, LNPs were made with various lipid components and at different ratios and were tested for their ability to transfect monocytes in vitro. For these experiments, the association of the LNP with cells and the expression of mRNA-encoding protein within the transfected cells was assessed at the cell-by-cell level. Peripheral blood mononuclear cells (PBMCs) from human donors were used. PBMCs were predominately T cells (40-60%) B cells (3-15%), and monocytes (15-35%), but also contained small numbers of other cell types. LNPs were incubated with serum or PBS for 30 minutes and then added to the PBMCs. The LNP concentration was 0.050 mg/ml, the LNP volume was 0.1 ml.

LNPs contained 0.1% Rhodamine-DOPE in addition to the other LNP components to facilitate detection. Thus, the ratios set forth in the tables below include 0.1 for the rhodamine label. For example, 50:10:10:27.9:2:0.1 AL:DSPC:chol:sterol:Cmp 428 indicates 50% amino lipid as the ionizable lipid, 10% DSPC as the phospholipid, 10% cholesterol, 27.9% phytosterol, 2% Compound 428 as the PEG lipid and 0.1% for the rhodamine-DOPE label. To prepare a non-labeled LNP, the same ratios are used except that the 0.1% rhodamine label is omitted and an additional 0.1% of structural lipid (cholesterol or cholesterol/phytosterol blend) is used (e.g., 27.9% becomes 28%).

Cells were sorted by flow cytometry into subsets based on expression of certain markers: CD3 (T cells), CD20 (B cells), and CD3−, CD20−, CD14+ (Monocytes).

mRNA encoding GFP was used as a model agent to effect expression of an intracellular protein.

When conducting flow cytometry, the Y-axis was signal from the rhodamine tag on the LNP and the X-axis was the GFP signal showing expression of the protein.

The “associated” population is any rhodamine+ and/or GFP+ cell and the percent of these cells was calculated by dividing by the total number of cells. The “expression” population is any GFP+ cell and the percent of these cells was calculated by dividing by the total number of cells. The percent of associated cells expressing was obtained by dividing the % expressing cells by the % associated cells.

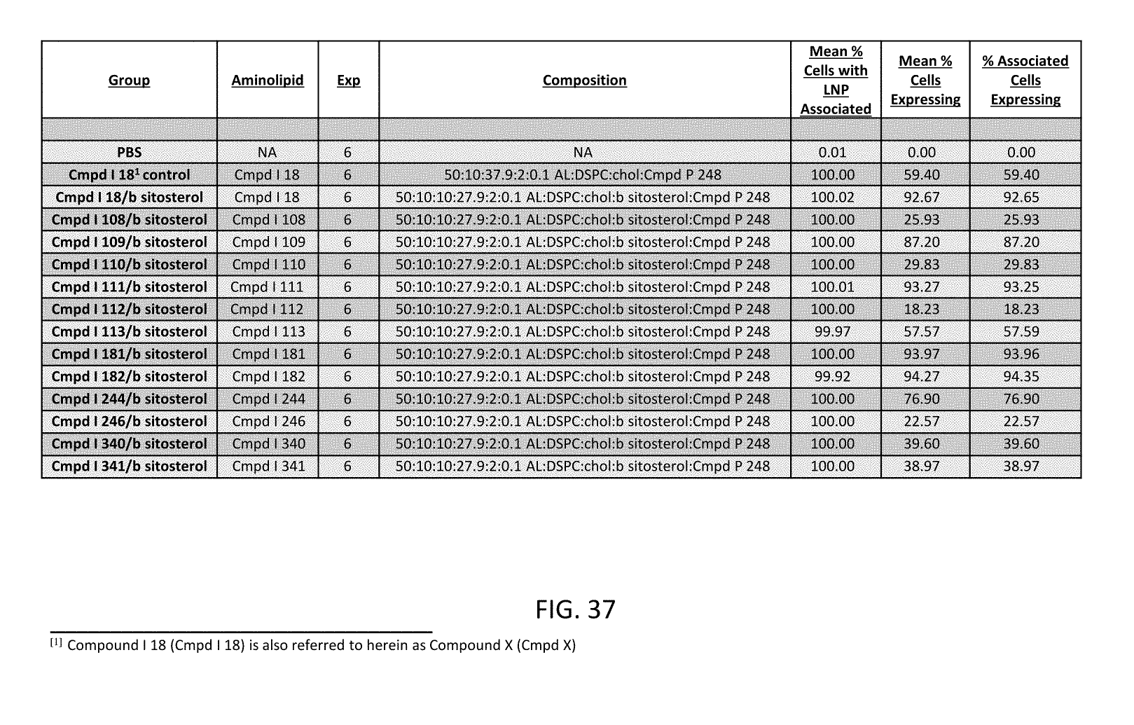

The results for a panel of amino lipids tested for the ionizable lipid component of the LNP are shown in FIG. 37. The results in FIG. 37 demonstrated that in particular amino lipid Compound X, Compound I-109, Compound I-111, Compound I-181, Compound I-182 and Compound I-244 exhibited immune cell delivery potentiating ability in monocytes.

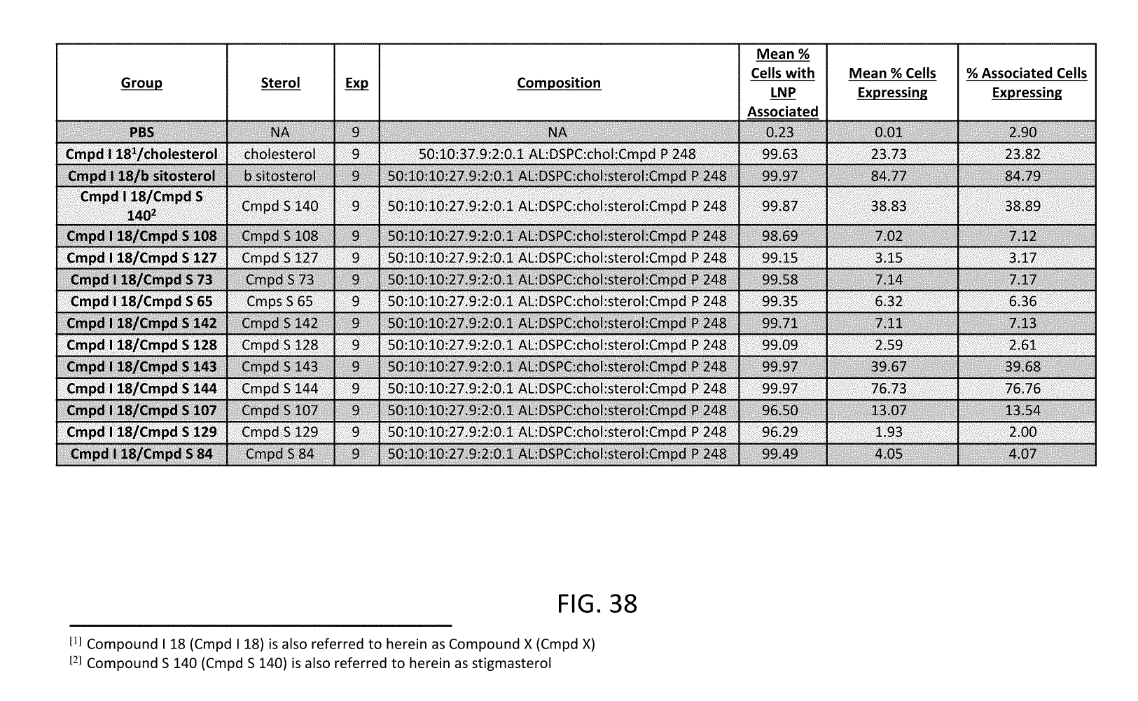

The results for a panel of sterols tested for the structural lipid component of the LNP are shown in FIG. 38. The results in FIG. 38 demonstrated that, in addition to β-sitosterol, in particular sterol compounds camposterol, β-sitostanol and stigmasterol exhibited immune cell delivery potentiating ability in monocytes.

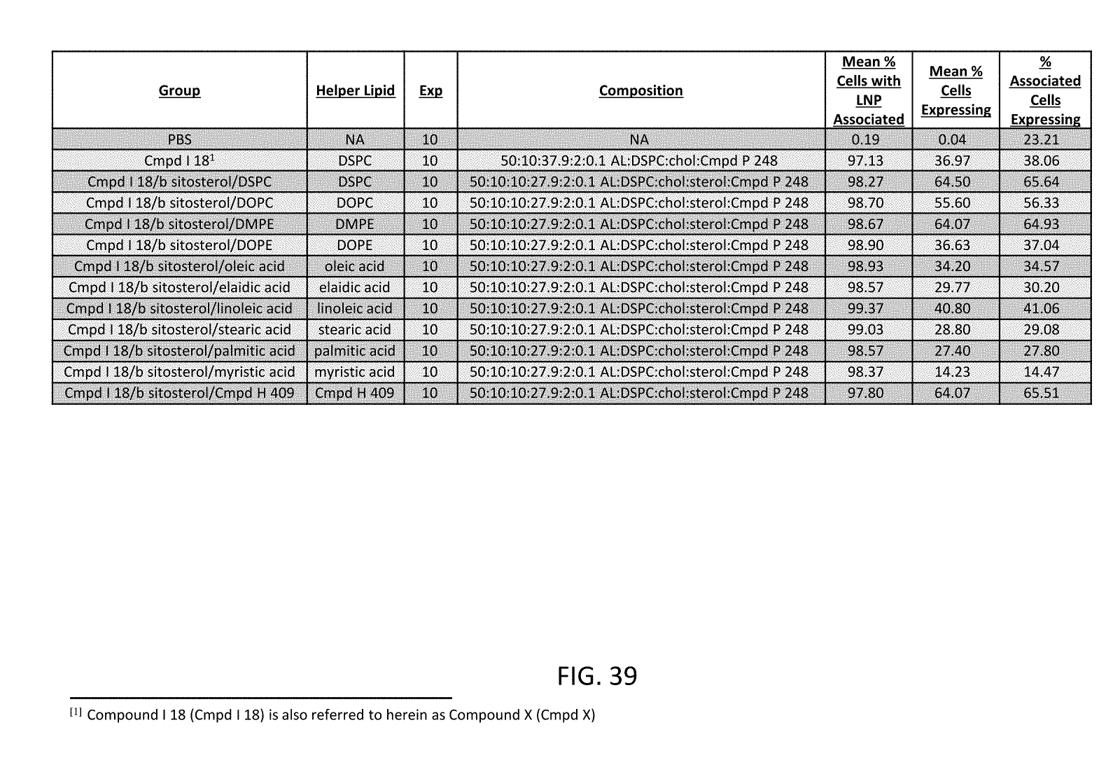

The results for a panel of helper lipids tested for the phospholipid component of the LNP are shown in FIG. 39. The results in FIG. 39 demonstrated that in particular phospholipid Compounds DOPC, DMPE and H-409 exhibited immune cell delivery potentiating ability in monocytes.

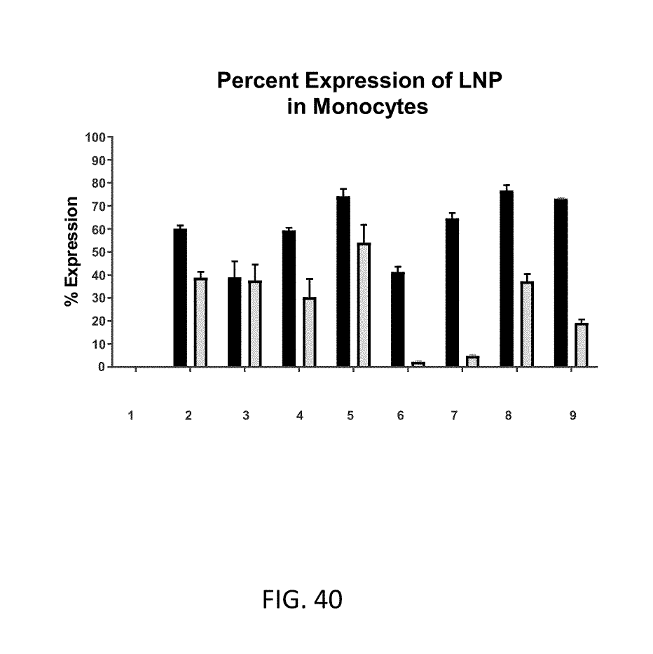

In a second series of experiments, an additional panel of sterol compounds was tested in LNPs for monocyte delivery in vivo. The results for this panel of sterols tested for the structural lipid component of the LNP are shown in the bar graph of FIG. 40. The Y-axis shows the percentage of CD3+ cells in the spleen that expressed mOX40L encoded by the mRNA encapsulated by the LNP. The bars/lanes on the X-axis labeled 1-9 correspond to the following LNP compositions tested for mRNA delivery in vivo: Lane 1 is PBS. Lane 2 is Cmpd X/Cmpd 428, Lane 3 is Cmpd X/β sitosterol/Cmpd 428, Lane 4 is Cmpd X/β-sitosterol-d7/Cmpd 428, Lane 5 is Cmpd X/brassicasterol/Cmpd 428, Lane 6 is Cmpd X/unnamed sterol/Cmpd 428, Lane 7 is Cmpd X/Cmpd S-30/Cmpd 428, Lane 8 is Cmpd X/Cmpd S-32/Cmpd 428, Lane 9 is Cmpd X/Cmpd S-31/Cmpd 428. These results demonstrated that LNPs containing sterol compounds brassicasterol (shown in Lane 5), Cmpd S-30 (shown in Lane 7) and Cmpd S-31 (shown in Lane 9) exhibited immune cell delivery potentiating ability in monocytes.

### Example 20: Transfection of T Cells with mRNA Encoding a Secreted Protein Using Immune Cell Delivery Potentiating Lipids

In this Example, mRNA encoding a secreted protein (human erythropoietin, huEPO) was encapsulated in an LNP containing immune cell delivery potentiating lipids (LNP 3, described in Example 3). Transfection of T cells in vitro by these LNPs was then compared to transfection of the same mRNA encapsulated in an LNP lacking the immune cell delivery potentiating lipids (LNP 1, described in Example 3).

For the huEPO transfection experiments, hPBMCs were thawed and washed in RPMI containing 10% fetal bovine serum (FBS). T cells were then sorted from the hPBMCs using a commercially available T cell isolation kit (STEMCELL Technologies). T cells were rested overnight n RPMI containing 10% FBS, then washed in RPMI and resuspended in 100 μl RPMI. Human serum was added to the cells to a final concentration of 2%. For transfection, 100 ng of LNP 1 or LNP 3 encapsulating mRNA encoding huEPO was added to the cells in 50 μl RPMI. Cells were incubated 24 hours at 37° C., followed by collection of supernatant. To measure huEPO concentration in the T cell supernatant, a commercially available human EPO ELISA kit (Thermo Fisher Scientific) was run at 1:50 and 1:250 supernatant dilutions.

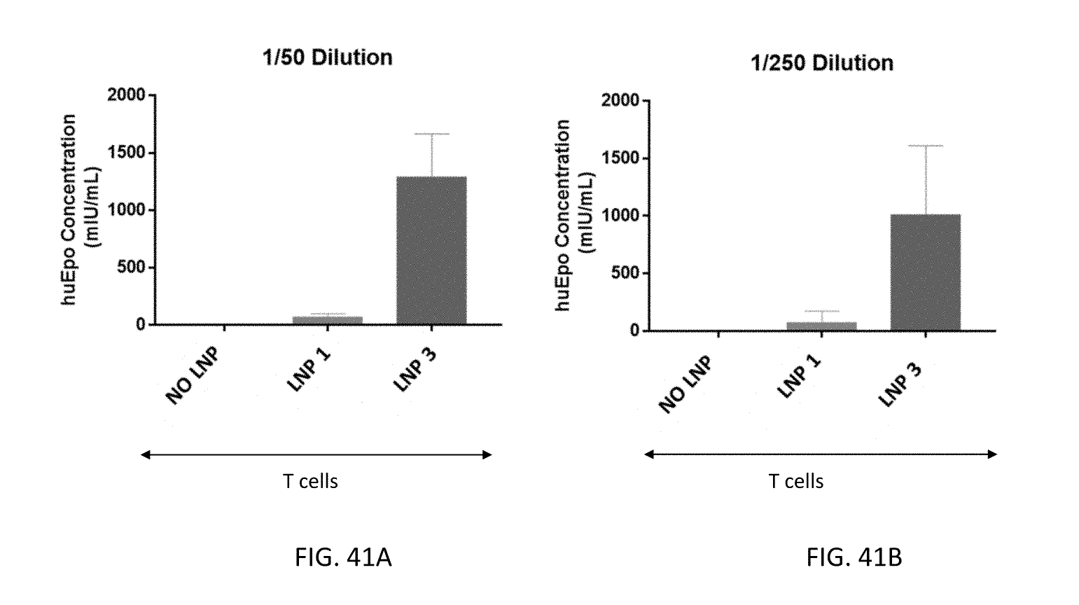

The results are shown in FIG. 41A (1:5 dilution) and 41B (1:250 dilution). The results demonstrate that use of the LNPs containing the immune cell delivery potentiating lipids (LNP 3) led to significantly higher levels of huEPO on the T cell supernatants as compared to use of LNPs lacking the immune cell delivery potentiating lipids (LNP 1). Thus, these experiments demonstrate that the LNPs containing the immune cell delivery potentiating lipids are effective at delivering mRNA encoding a secreted protein into T cells such that the secreted protein is expressed by the T cells.

### Example 21: Transfection of T Cells with mRNA Encoding an Intracellular Protein Using Immune Cell Delivery Potentiating Lipids

In this Example, mRNA encoding an intracellular protein (the transcription factor Foxp3) was encapsulated in an LNP containing immune cell delivery potentiating lipids (LNP 3, described in Example 3). Transfection of T cells in vitro by these LNPs was then compared to transfection of mRNA encapsulated in an LNP lacking the immune cell delivery potentiating lipids (LNP 1, described in Example 3).

For the Foxp3 transfection experiments, hPBMCs were thawed and washed in RPMI containing 10% fetal bovine serum (FBS) and T cells were then sorted from the hPBMCs using a commercially available T cell isolation kit (STEMCELL Technologies). T cells were rested overnight n RPMI containing 10% FBS, then washed in RPMI and resuspended in 100 μl RPMI. Human serum was added to the cells to a final concentration of 2%. For transfection, 100 ng of LNP 1 or LNP 3 encapsulating mRNA encoding Foxp3 was added to the cells in 50 μl RPMI. mRNA encoding the membrane-bound protein mOX40L encapsulated in LNP 3 was used as a positive control.

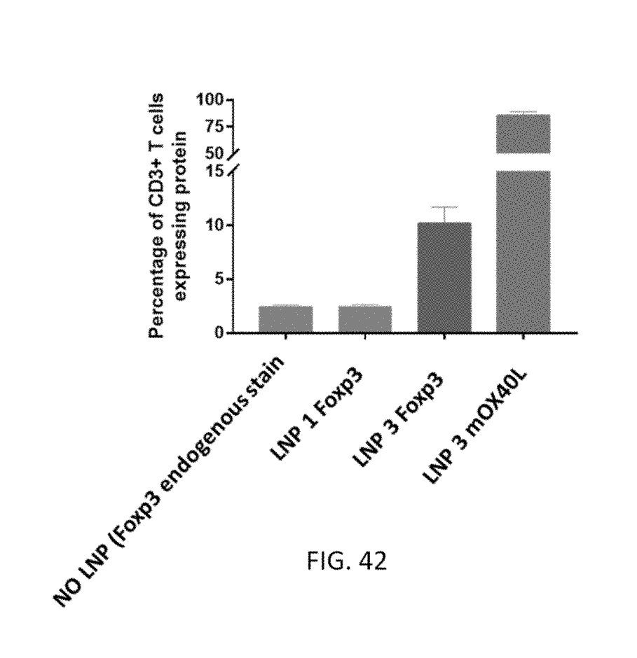

The results are shown in FIG. 42. The results demonstrate that use of the LNPs containing the immune cell delivery potentiating lipids (LNP 3) led to significantly higher levels of expression of Foxp3 in CD3+ T cells as compared to use of LNPs lacking the immune cell delivery potentiating lipids (LNP 1). Thus, these experiments demonstrate that the LNPs containing the immune cell delivery potentiating lipids are effective at delivering mRNA encoding an intracellular protein (e.g., a transcription factor) into T cells such that the intracellular protein is expressed by the T cells.

### Example 22: Additional Lipids that are Immune Cell Delivery Potentiating Lipids for Transfection of Immune Cells

In this Example, additional LNPs comprising various lipid components and at different ratios were made and tested for their ability to transfect T cells in vitro. Transfection was performed as in Example 5 using LNPs encapsulating an mRNA encoding mouse OX40L. LNPs were labeled and the results were determined for the mean % cells with LNPs associated, the mean % cells expressing mOX40L and the % of LNP-associated cells expressing mOX40L, as described hereinbefore.

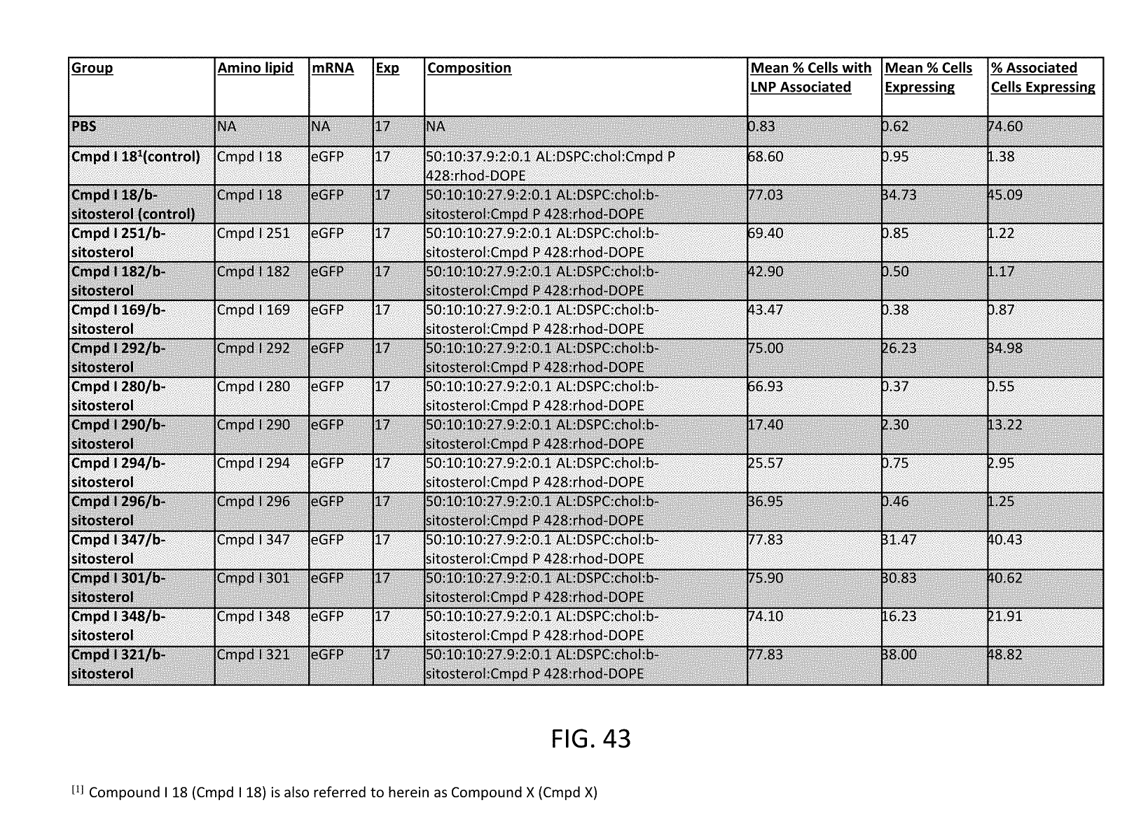

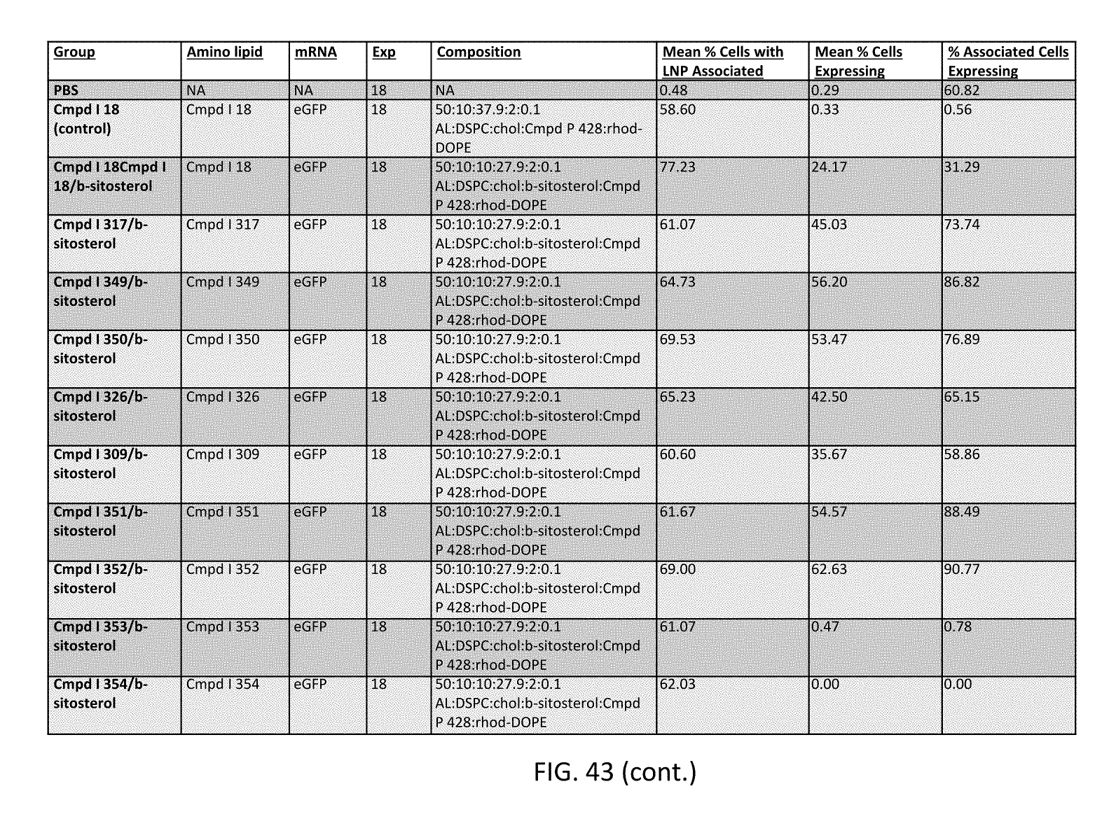

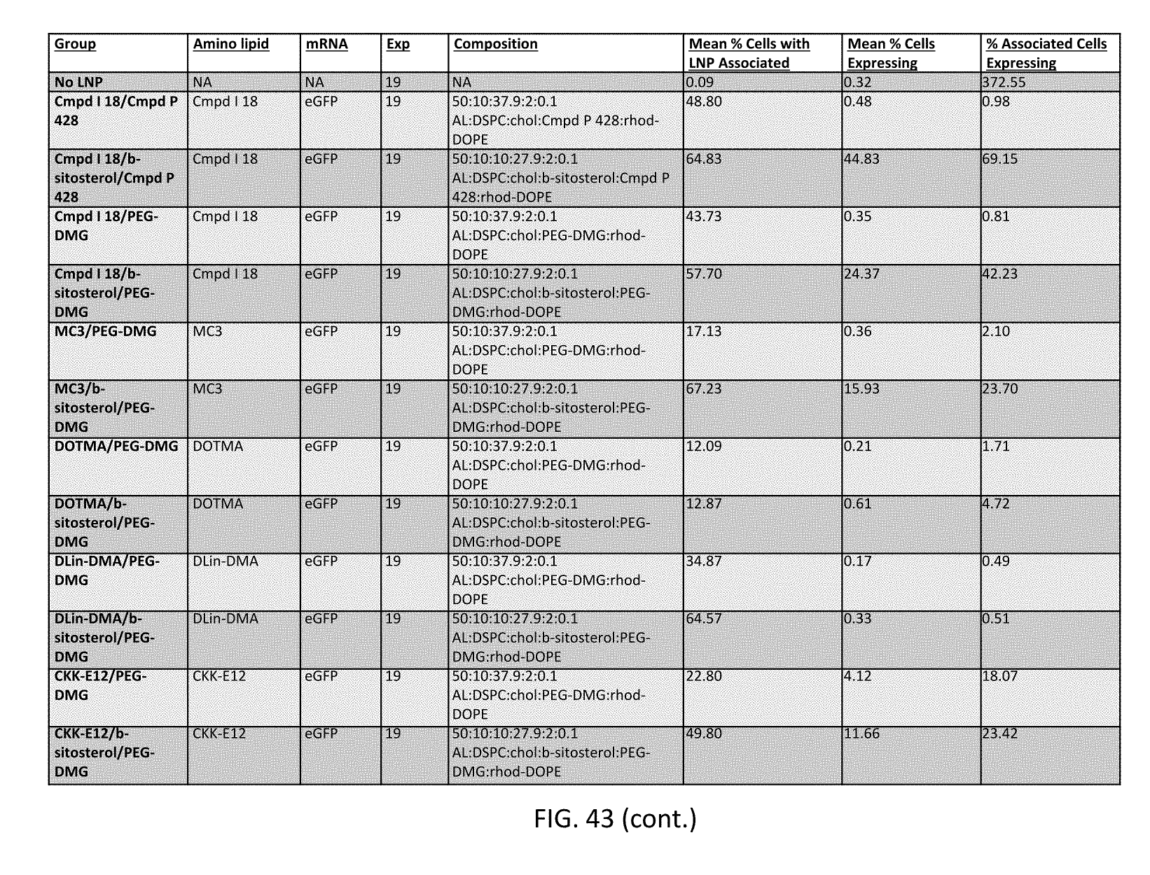

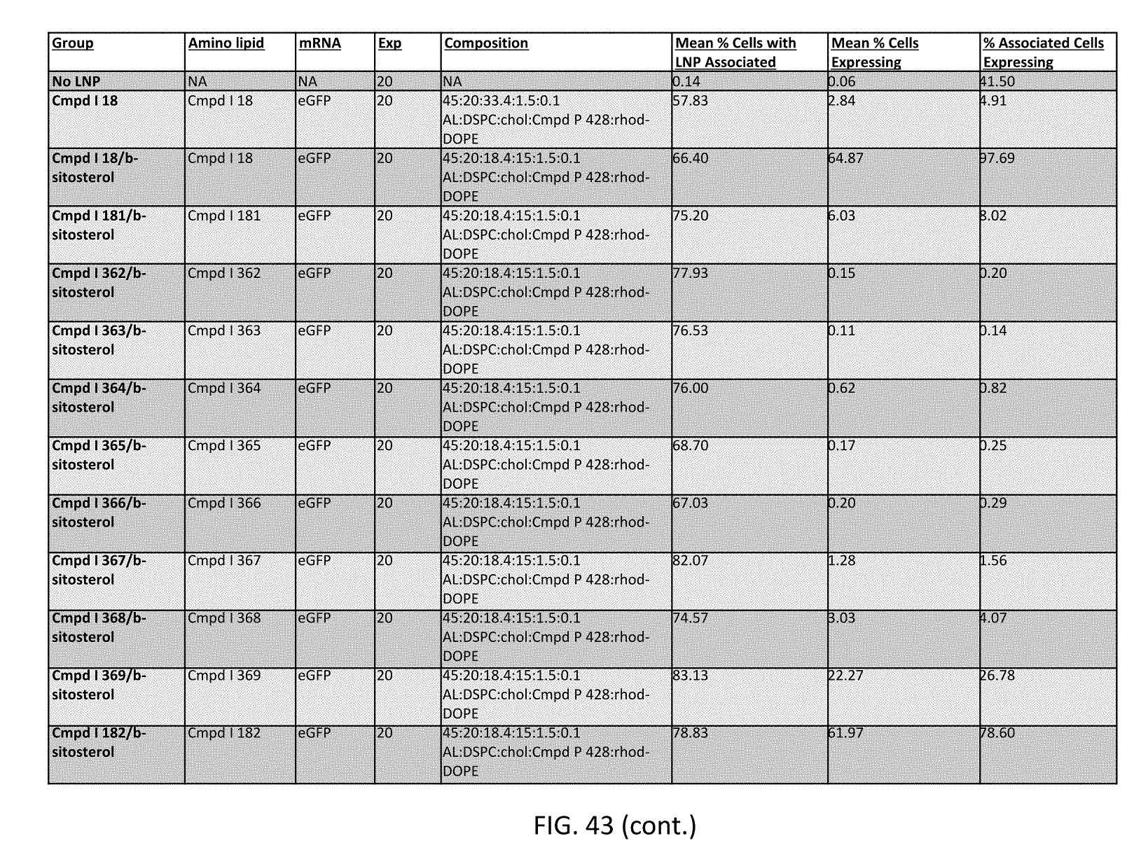

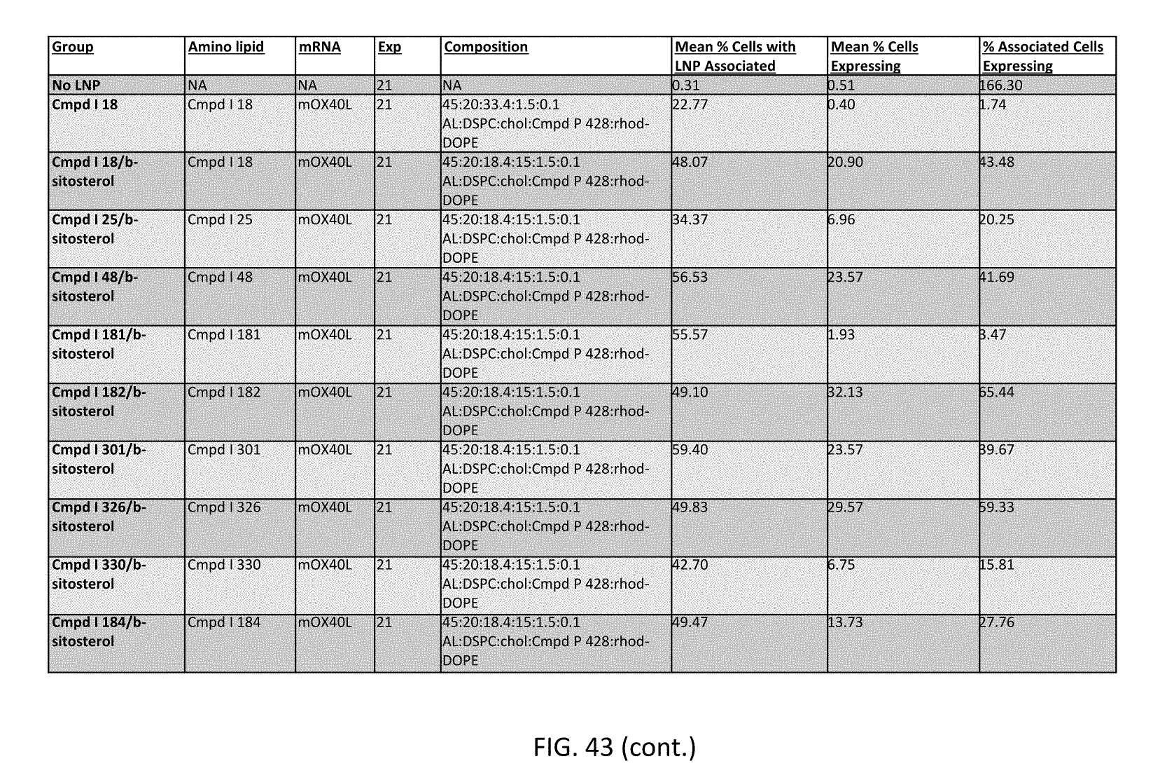

The results for a panel of amino lipids tested for the ionizable lipid component of the LNP are shown in FIG. 43. The results in FIG. 43 demonstrated that in particular amino lipid Compound I-48, Compound I-181, Compound I-182, Compound I-292, Compound I-301, Compound I-309, Compound I-317, Compound I-321, Compound I-326, Compound I-347, Compound I-348, Compound I-349, Compound I-350, Compound I-351 and Compound I-352 exhibited immune cell delivery potentiating ability in T cells.

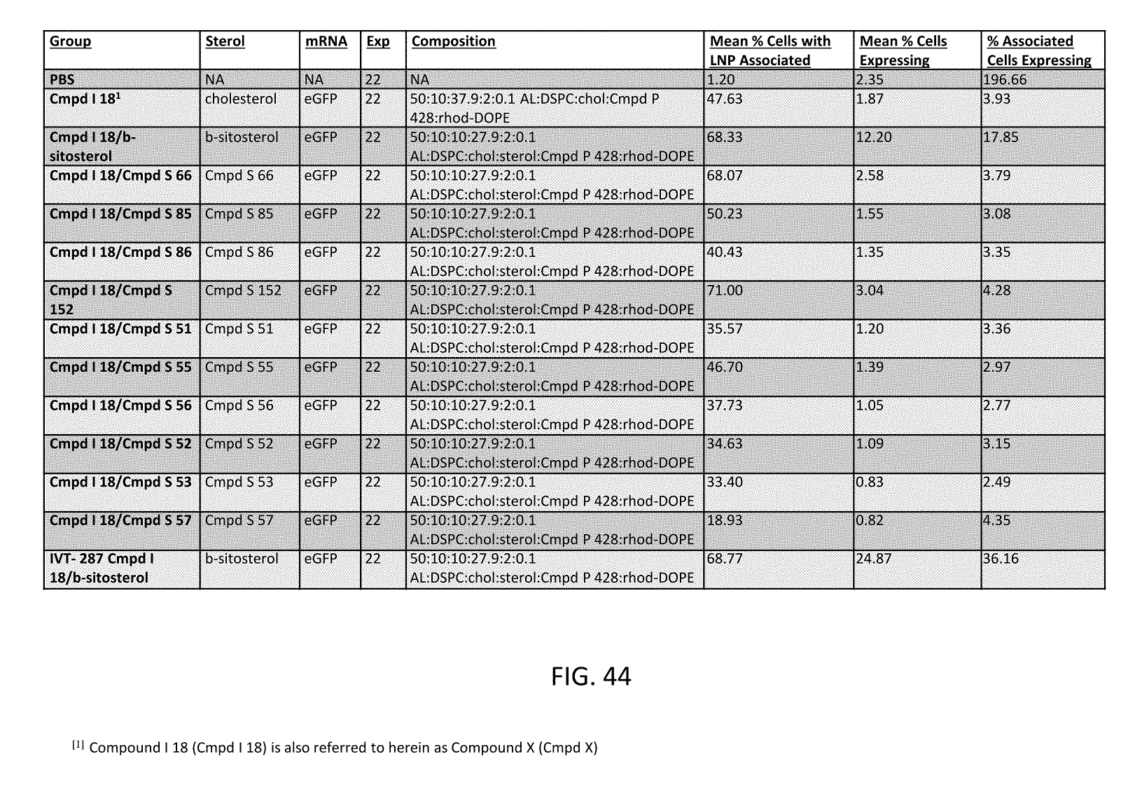

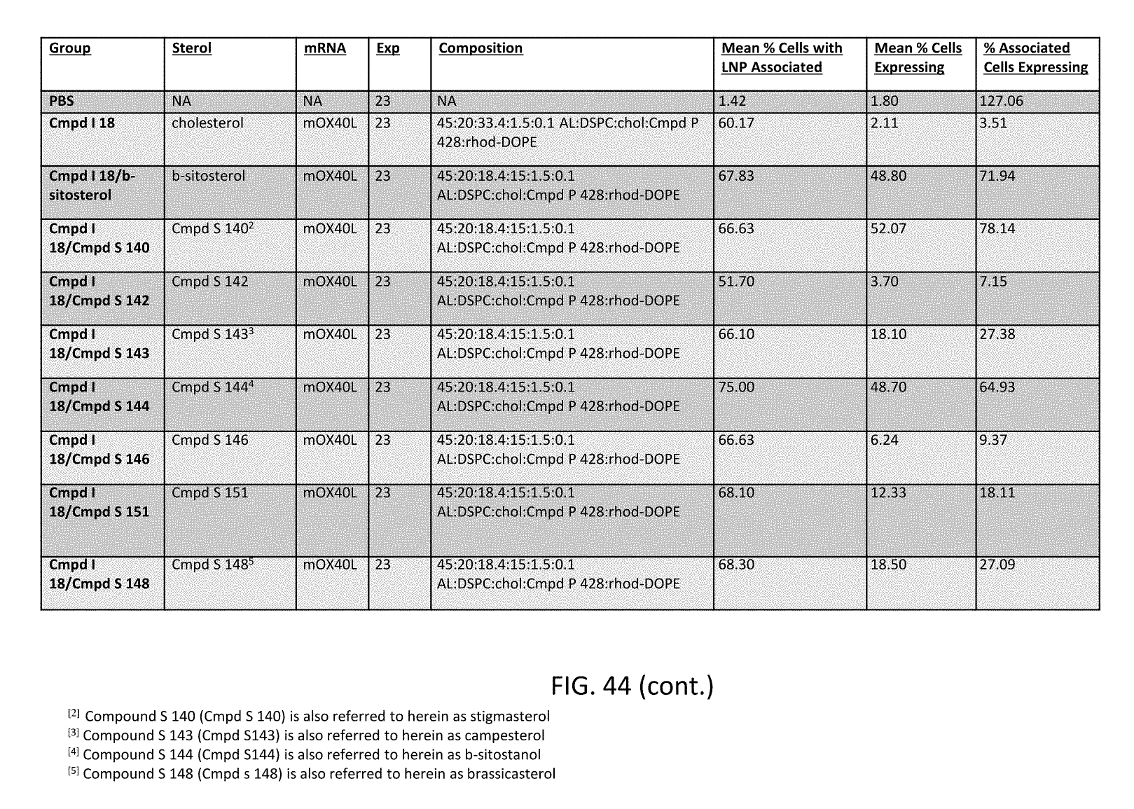

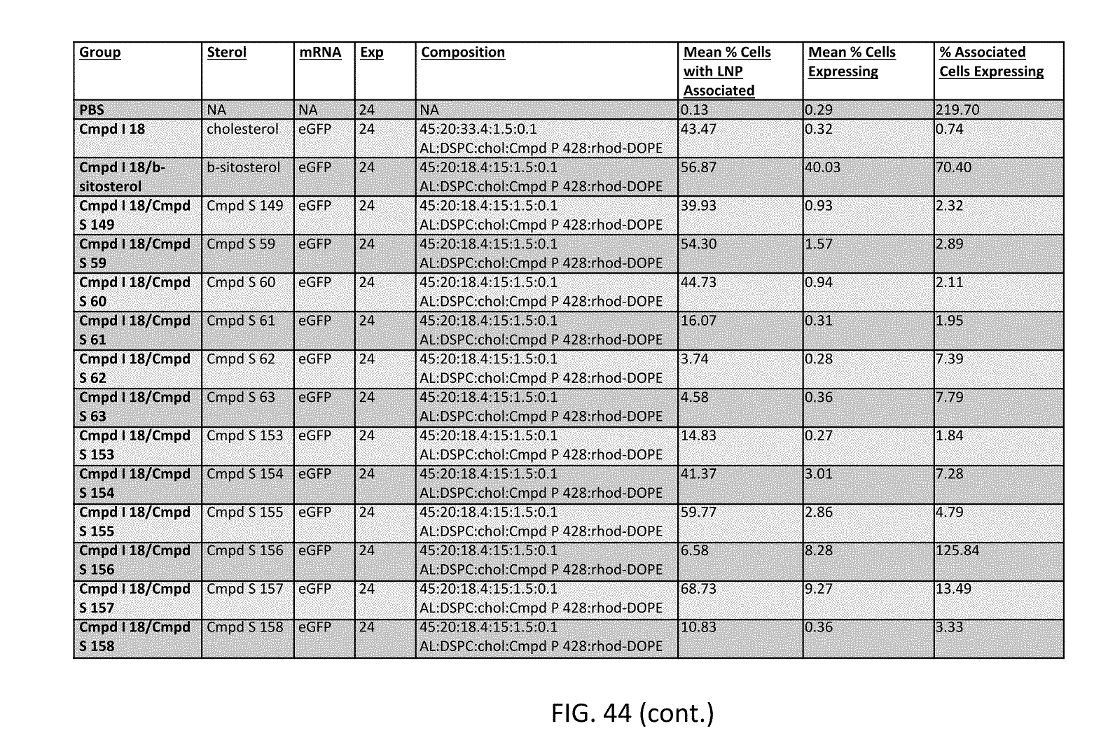

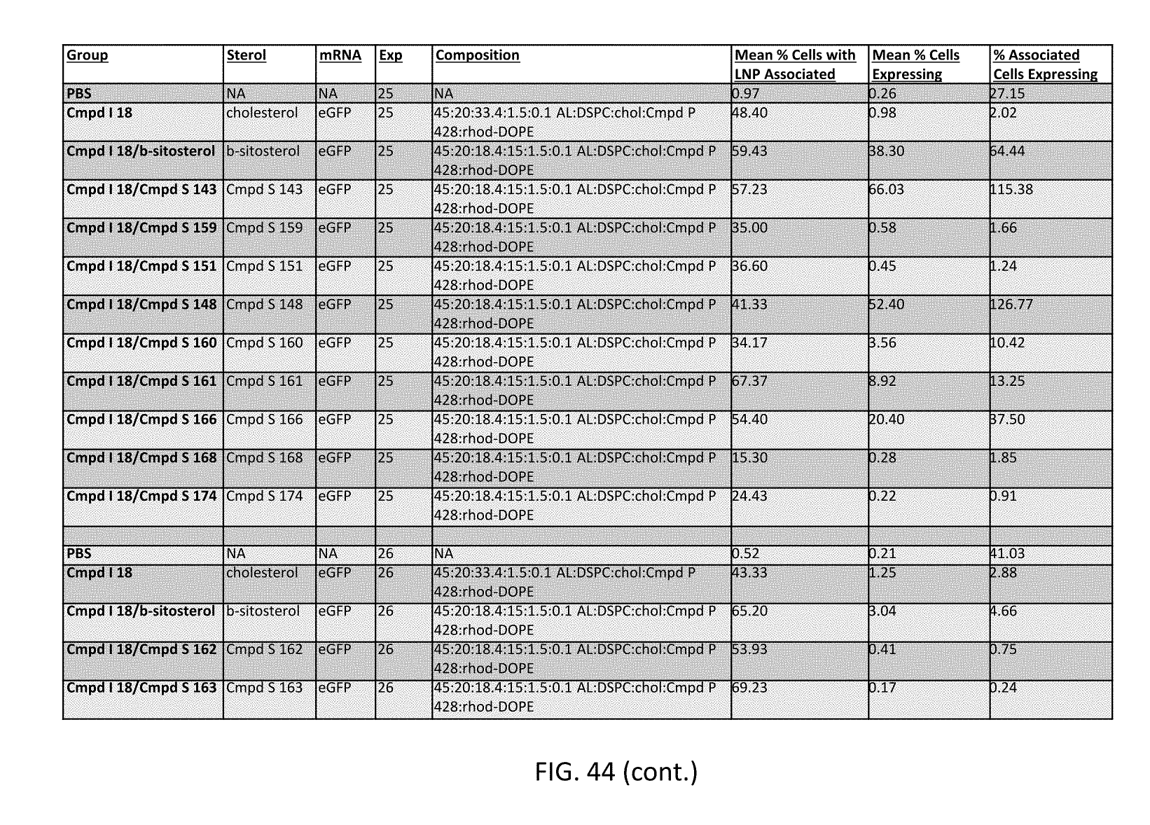

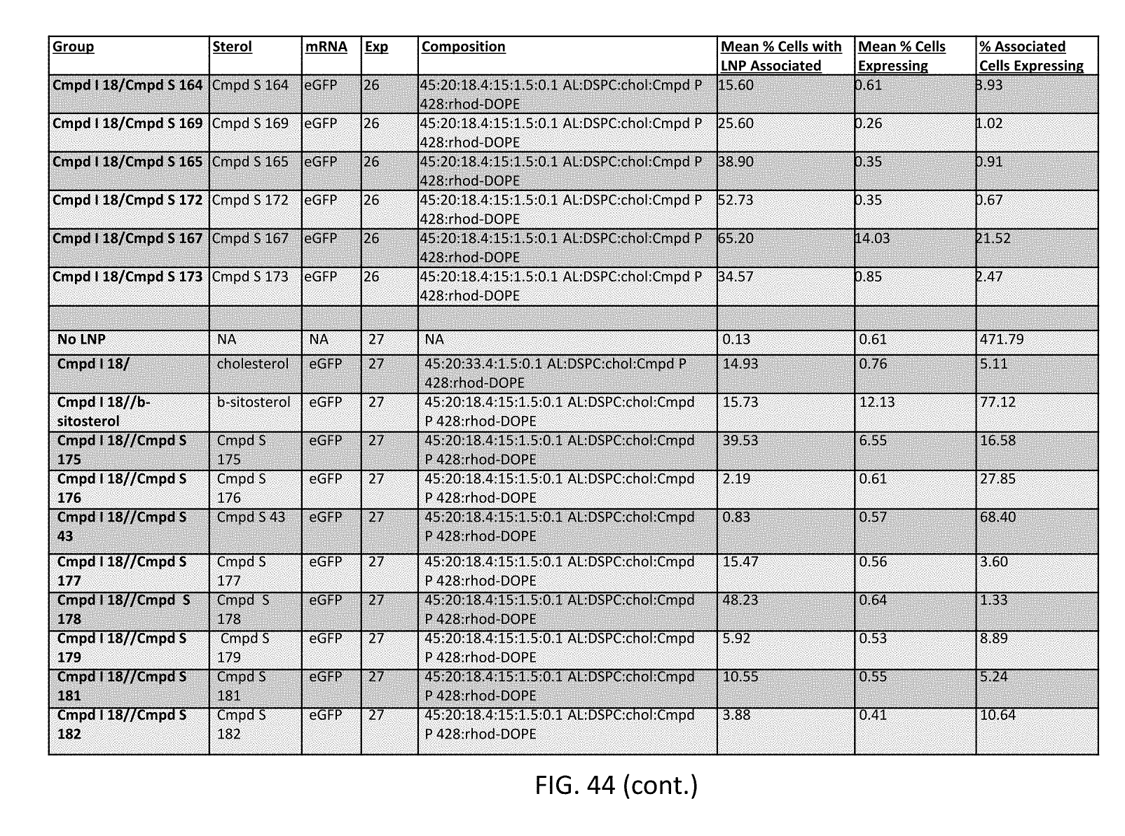

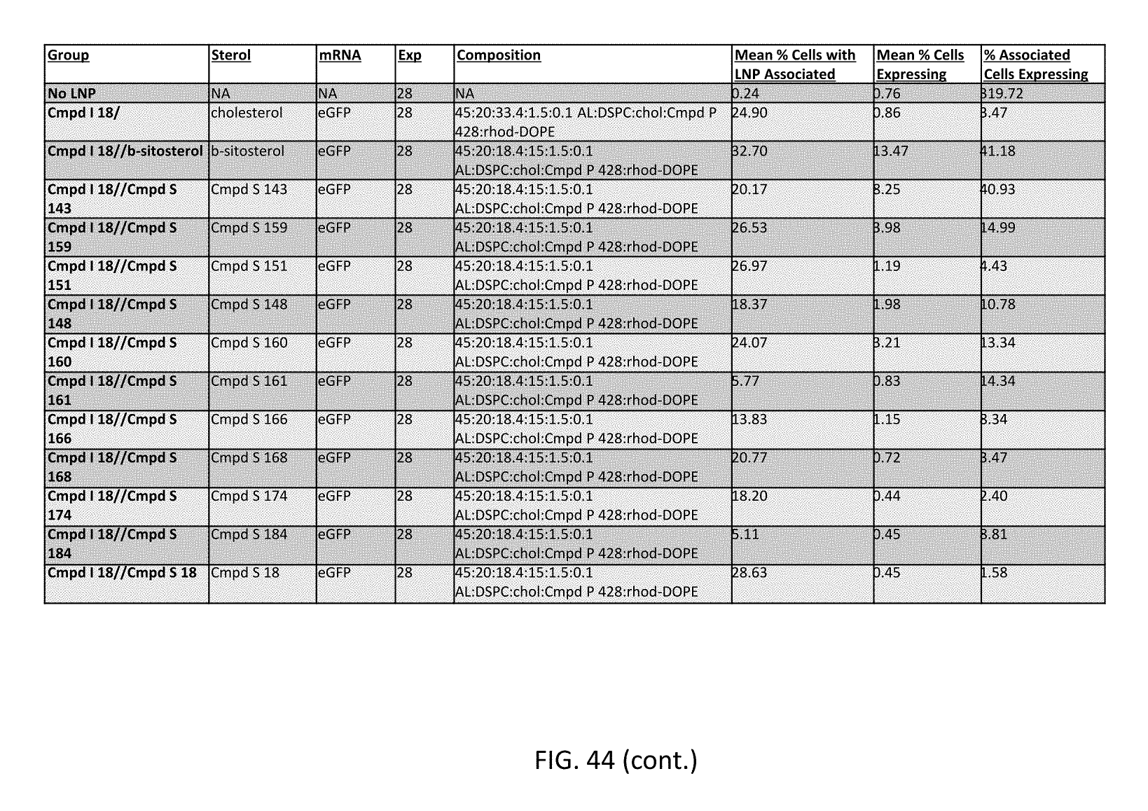

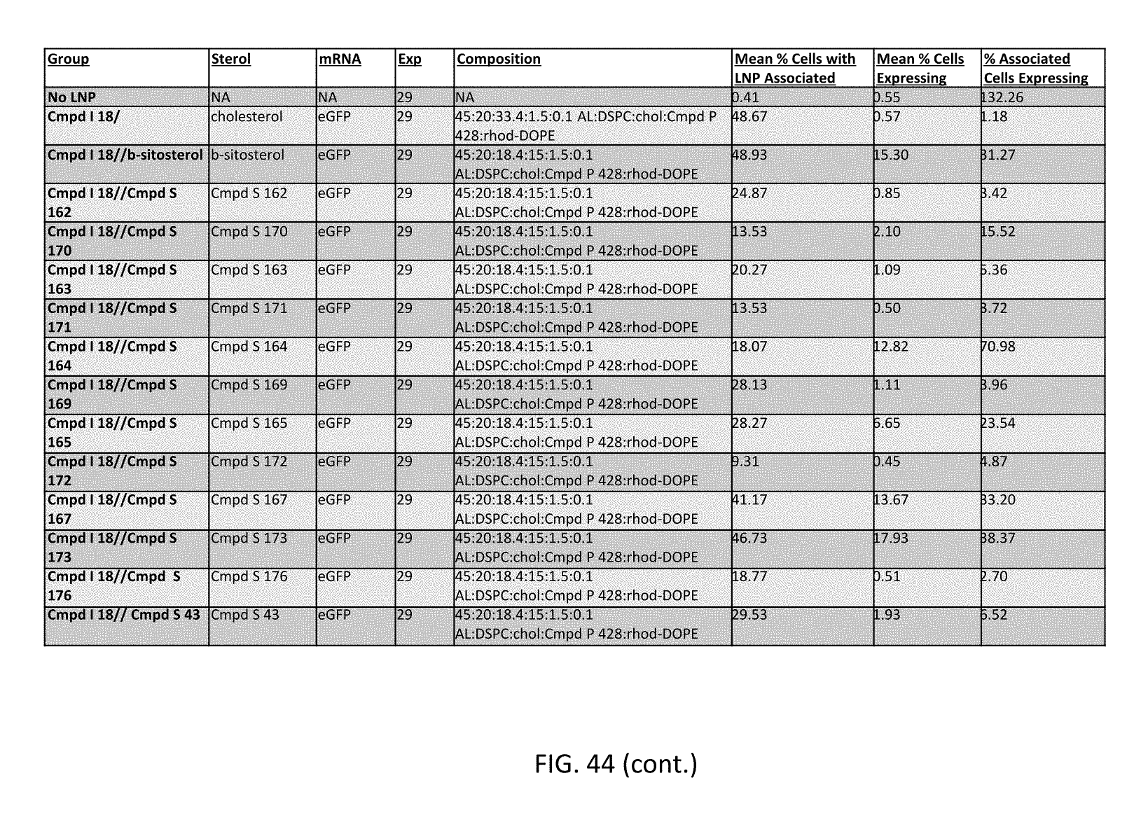

The results for a panel of sterols tested as the structural component of the LNP are shown in FIG. 44. The results in FIG. 44 demonstrated that in particular sterol Compound S-140, Compound S-143, Compound S-144, Compound S-148, Compound S-151, Compound S-156, Compound S-157, Compound S-159, Compound S-160, Compound S-164, Compound S-165, Compound S-166, Compound S-167, Compound S-170, Compound S-173 and Compound S-175 exhibited immune cell delivery potentiating ability in T cells.

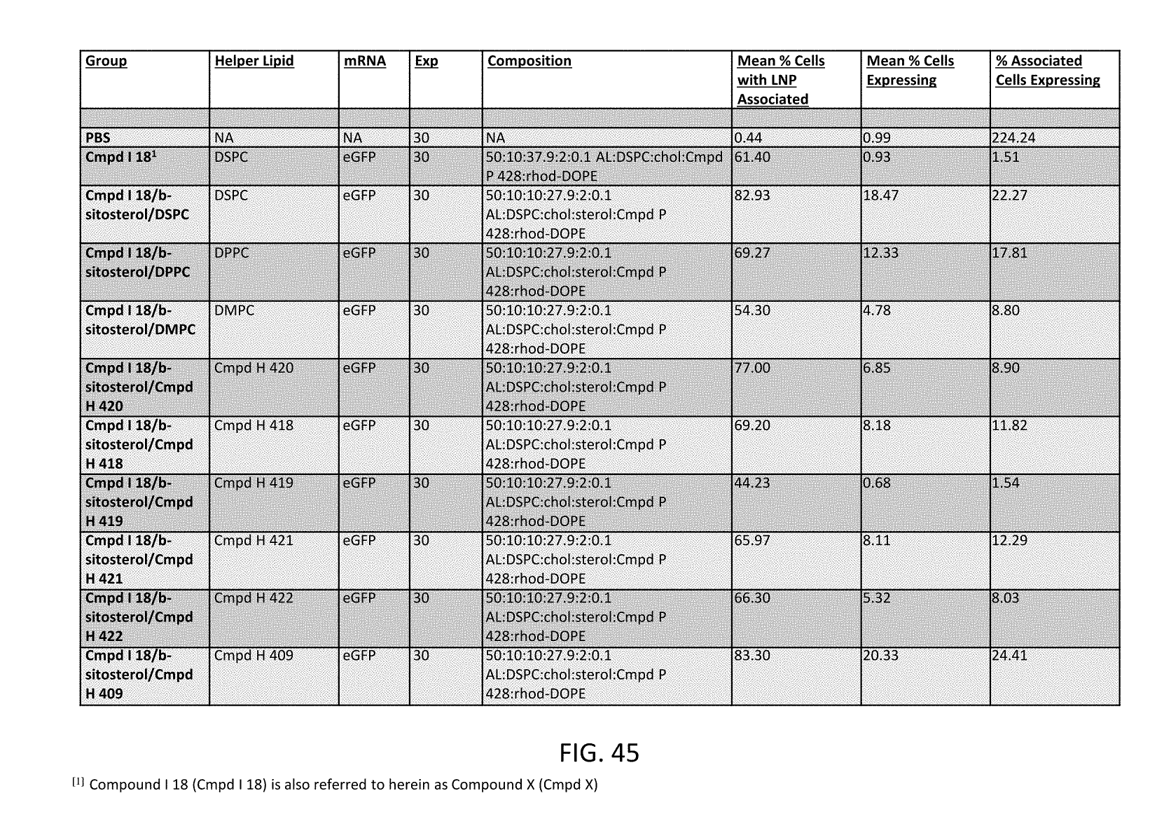

The results for a panel of helper lipids tested as the phospholipid component of the LNP are shown in FIG. 45. The results in FIG. 45 demonstrated that in particular helper lipid DPPC, DMPC, Compound H-418, Compound H-420, Compound H-421, Compound H-422 and Compound H-409 exhibited immune cell delivery potentiating ability in T cells.

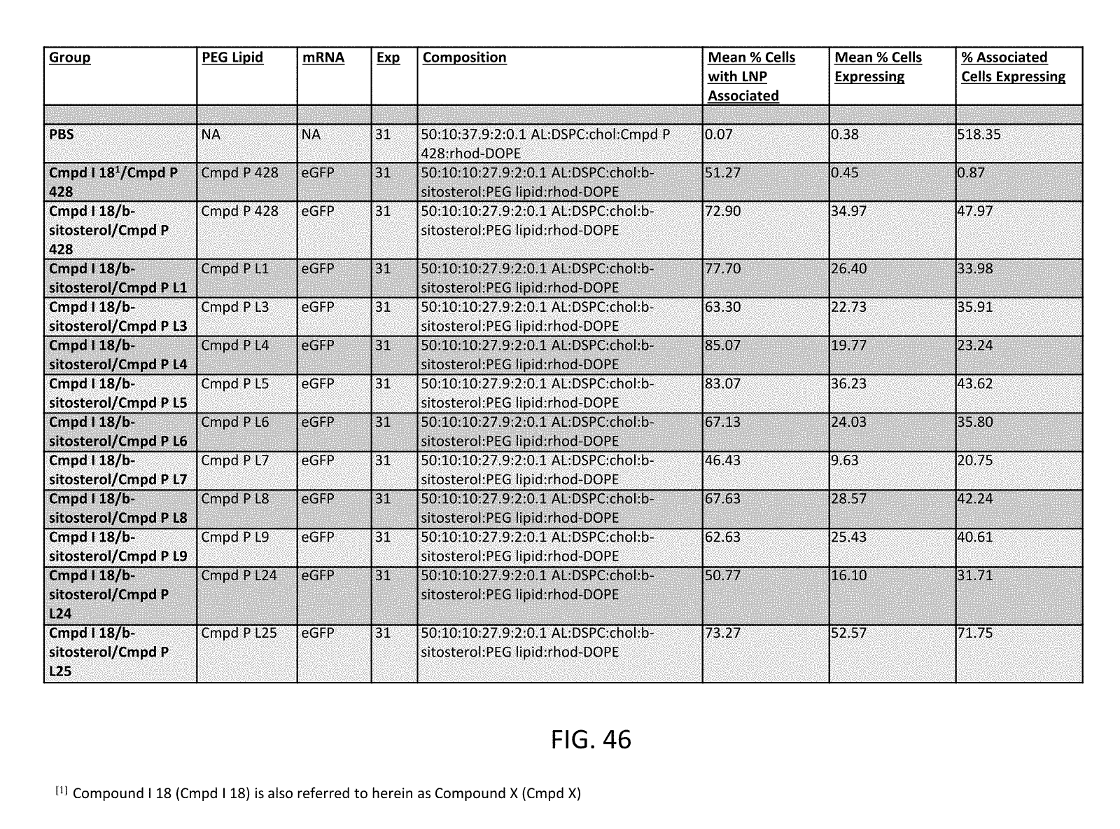

The results of a panel of PEG lipids tested as the PEG lipid component of the LNP are shown in FIG. 46. The results in FIG. 46 demonstrated that in particular PEG lipid Compound P-L1, Compound P-L3, Compound P-L4, Compound P-L6, Compound P-L8, Compound P-L9, and Compound P-L25 exhibited immune cell delivery potentiating ability in T cells.

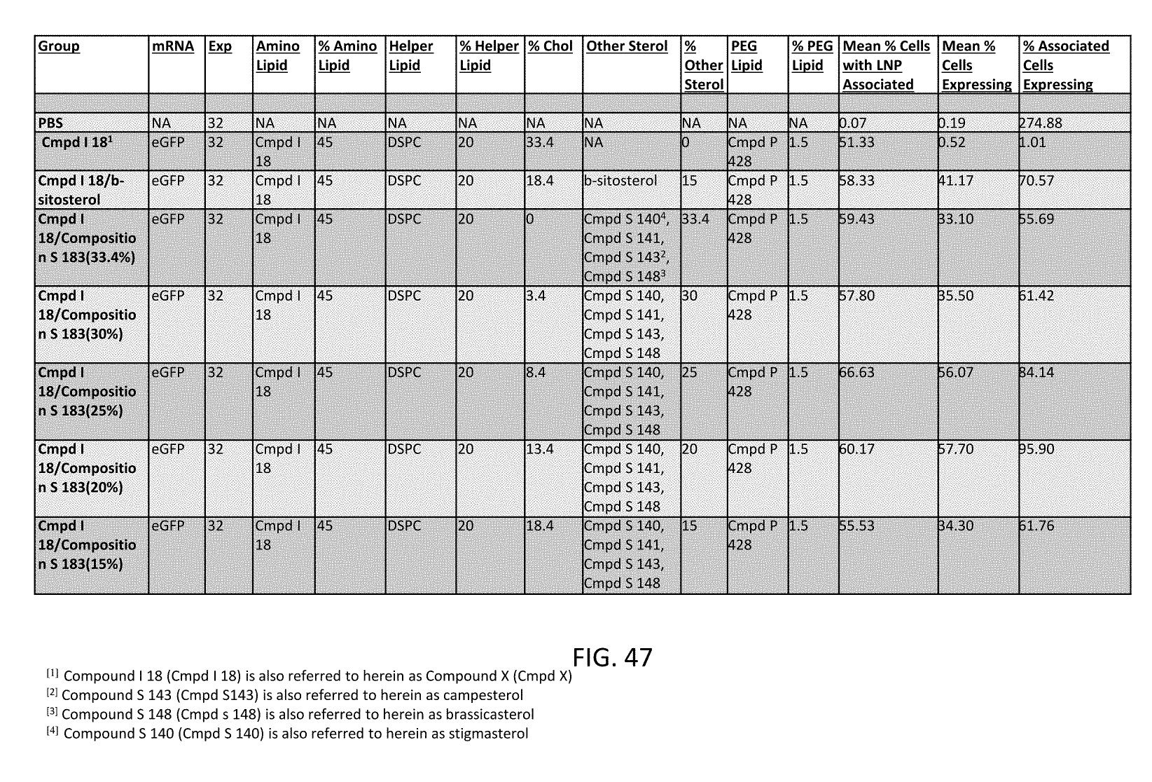

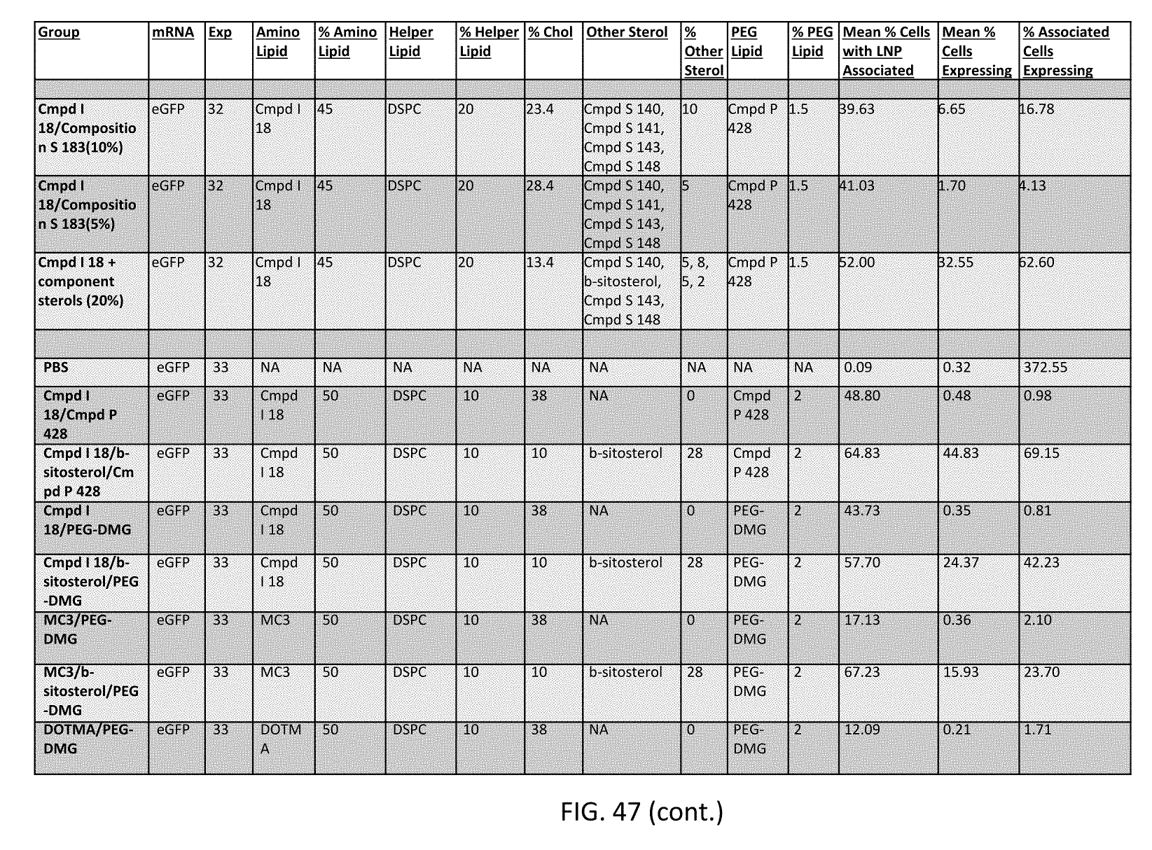

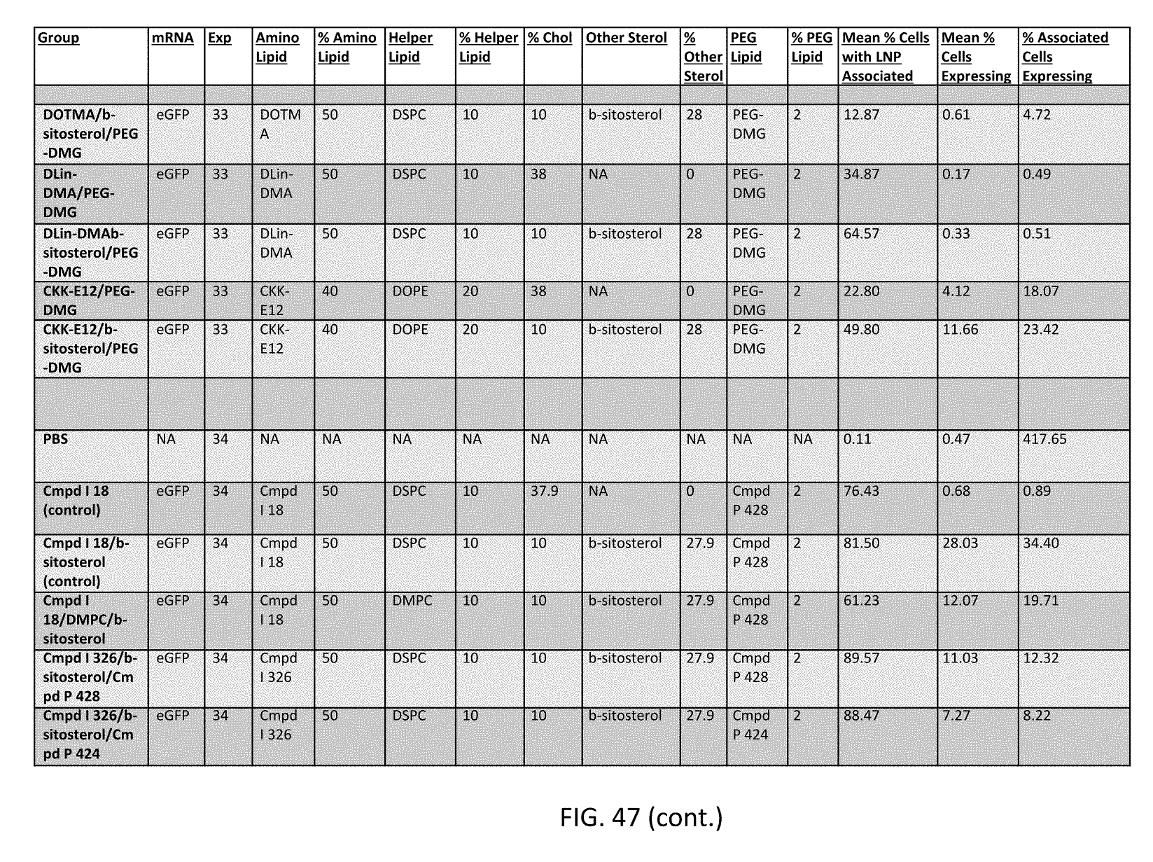

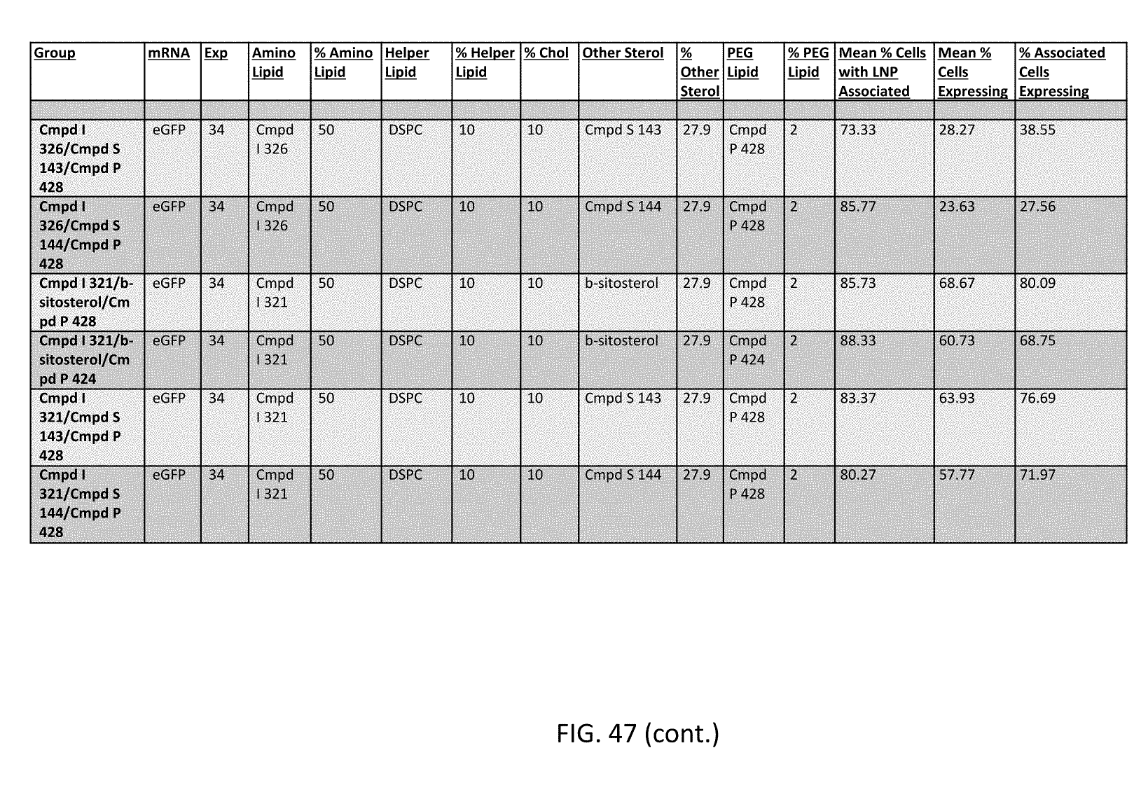

The results of a panel of different formulations of the LNP composition are shown in FIG. 47. This figure reports results from three different experiments tested three different types of formulation, as follows.

In a first series of experiments, a panel of formulations containing Compound I-182 as the ionizable lipid (amino lipid) was compared to formulations containing Compound I-18 as the ionizable lipid (amino lipid). These experiments demonstrated that the Compound I-182-containing LNPs exhibited immune cell delivery potentiating ability in T cells. In particular, the following formulations/ratios were particularly effective in immune cell delivery potentiating ability in T cells: Compound I-182/DSPC/cholesterol/P-428 at a ratio of 40:30:28:2; and Compound I-182/ISPC/cholesterol/β-sitosterol/P-428 at any of the following ratios: 50:10:20:18:2; or 50:20:10:18:2; or 40:20:20:18:2.

In a second series of experiments, a panel of formulations containing cholesterol/Composition S-183 (˜40% Compound S-141, ˜25% Compound S-140, ˜25% Compound S-143 and ˜10% brassicasterol) as the structural component was tested against formulations having cholesterol/β-sitosterol as the structural component. The ratio used for the formulations was 45% ionizable lipid/20% helper lipid/33.5% structural lipid/1.5% PEG lipid. These experiments demonstrated that formulations containing cholesterol/Composition S-183 exhibited immune cell delivery potentiating ability in T cells. Formulations were most effective when they included at least 15% Composition S-183 in the structural lipid component (e.g., 15%, 20%, 25%, 30% or 33.5% Composition S-183, with cholesterol making up 18.5%, 13.5%, 8.5%, 3.5% or 0% of the structural lipid). Furthermore, a formulation comprising 45% Compound I-18/20% DSPC/13.5% cholesterol/20% mixture of four different sterols/1.5% P-428 was also effective immune cell delivery potentiating ability in T cells. The 20% mixture of four sterols comprised: 5% Compound S-140, 8%/β-sitosterol, 5% Compound S-143 and 2% Compound S-148.

In a third series of experiments, a panel of formulations containing various different ionizable lipids (amino lipids) was tested against formulations having Compound I-18 as the ionizable lipid (amino lipid). The results demonstrated that formulations comprising MC3 (((6Z,9Z,28Z,31Z)-heptatriaconta-6,9,28,31-tetraen-19-yl 4-(dimethylamino)butanoate); U.S. Pat. No. 8,158,601, herein incorporated by reference) or CKK-E12 (Dong, Y. et al. PNAS Mar. 19, 2014, Vol 111 (11): 3955-3960, herein incorporated by reference) as the ionizable lipid (amino lipid) exhibited immune cell delivery potentiating ability in T cells.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

SEQUENCE LISTING SUMMARY

| SEQ | |
|---|---|
| ID NO | SEQUENCE |
| 1 | MHQKRTAMFQDPQER |
| | (HPV E6 peptide) |
| 2 | RTAMFQDPQERPRKL |
| | (HPV E6 peptide) |
| 3 | FQDPQERPRKLPQLC |
| | (HPV E6 peptide) |
| 4 | QERPRKLPQLCTELQ |
| | (HPV E6 peptide) |
| 5 | RKLPQLCTELQTTIH |
| | (HPV E6 peptide) |
| 6 | QLCTELQTTIHDIIL |
| | (HPV E6 peptide) |
| 7 | ELQTTIHDIILECVY |
| | (HPV E6 peptide) |
| 8 | LECVYCKQQLLRRII |
| | (HPV E6 peptide) |
| 9 | IILECVYCKQQLLRR |
| | (HPV E6 peptide) |
| 10 | CVYCKQQLLRREVYD |
| 11 | KQQLLRREVYDFAFR |
| | (HPV E6 peptide) |
| 12 | LRREVYDFAFRDLCI |
| | (HPV E6 peptide) |
| 13 | VYDFAFRDLCIVYRD |
| | (HPV E6 peptide) |
| 14 | AFRDLCIVYRDGNPY |
| | (HPV E6 peptide) |
| 15 | LCIVYRDGNPYAVCD |
| | (HPV E6 peptide) |
| 16 | YRDGNPYAVCDKCLK |
| | (HPV E6 peptide) |
| 17 | NPYAVCDKCLKFYSK |
| | (HPV E6 peptide) |
| 18 | VCDKCLKFYSKISEY |
| | (HPV E6 peptide) |
| 19 | CLKFYSKISEYRHYC |
| | (HPV E6 peptide) |
| 20 | YSKISEYRHYCYSLY |
| | (HPV E6 peptide) |
| 21 | SEYRHYCYSLYGTTL |
| | (HPV E6 peptide) |
| 22 | HYCYSLYGTTLEQQY |
| | (HPV E6 peptide) |
| 23 | SLYGTTLEQQYNKPL |
| | (HPV E6 peptide) |
| 24 | TTLEQQYNKPLCDLL |
| | (HPV E6 peptide) |
| 25 | QQYNKPLCDLLIRCI |
| | (HPV E6 peptide) |
| 26 | KPLCDLLIRCINCQK |
| | (HPV E6 peptide) |
| 27 | DLLIRCINCQKPLCP |
| | (HPV E6 peptide) |
| 28 | RCINCQKPLCPEEKQ |
| | (HPV E6 peptide) |
| 29 | CQKPLCPEEKQRHLD |
| | (HPV E6 peptide) |
| 30 | LCPEEKQRHLDKKQR |
| | (HPV E6 peptide) |
| 31 | EKQRHLDKKQRFHNI |
| | (HPV E6 peptide) |
| 32 | HLDKKQRFHNIRGRW |
| | (HPV E6 peptide) |
| 33 | KQRFHNIRGRWTGRC |
| | (HPV E6 peptide) |
| 34 | HNIRGRWTGRCMSCC |
| | (HPV E6 peptide) |
| 35 | GRWTGRCMSCCRSSR |
| | (HPV E6 peptide) |
| 36 | GRCMSCCRSSRTRRE |
| | (HPV E6 peptide) |
| 37 | SCCRSSRTRRETQL |
| | (HPV E6 peptide) |
| 38 | MHGDTPTLHEYMLDL |
| | (HPV E7 peptide) |
| 39 | TPTLHEYMLDLQPET |
| | (HPV E7 peptide) |
| 40 | HEYMLDLQPETTDLY |
| | (HPV E7 peptide) |
| 41 | LDLQPETTDLYCYEQ |
| | (HPV E7 peptide) |
| 42 | PETTDLYCYEQLNDS |
| | (HPV E7 peptide) |
| 43 | DLYCYEQLNDSSEEE |
| | (HPV E7 peptide) |
| 44 | YEQLNDSSEEEDEID |
| | (HPV E7 peptide) |
| 45 | NDSSEEEDEIDGPAG |
| | (HPV E7 peptide) |
| 46 | EEEDEIDGPAGQAEP |
| | (HPV E7 peptide) |
| 47 | EIDGPAGQAEPDRAH |
| | (HPV E7 peptide) |
| 48 | PAGQAEPDRAHYNIV |
| | (HPV E7 peptide) |
| 49 | AEPDRAHYNIVTFCC |
| | (HPV E7 peptide) |
| 50 | RAHYNIVTFCCKCDS |
| | (HPV E7 peptide) |
| 51 | NIVTFCCKCDSTLRL |
| | (HPV E7 peptide) |
| 52 | FCCKCDSTLRLCVQS |
| | (HPV E7 peptide) |
| 53 | CDSTLRLCVQSTHVD |
| | (HPV E7 peptide) |
| 54 | LRLCVQSTHVDIRTL |
| | (HPV E7 peptide) |
| 55 | VQSTHVDIRTLEDLL |
| | (HPV E7 peptide) |
| 56 | HVDIRTLEDLLMGTL |
| | (HPV E7 peptide) |
| 57 | RTLEDLLMGTLGIVC |
| | (HPV E7 peptide) |
| 58 | DLLMGTLGIVCPICS |
| | (HPV E7 peptide) |
| 59 | GTLGIVCPICSQKP |
| | (HPV E7 peptide) |
| 60 | TCAAGCTTTTGGACCCTCGTACAGAAGCTAATACGACTCAC |
| | TATAGGGAAATAAGAGAGAAAAGAAGAGTAAGAAGAAATATAA |
| | GAGCCACC |
| | (5′ UTR) |
| 61 | TGATAATAGGCTGGAGCCTCGGTGGCCATGCTTCTTGCCCCTTG |
| | GGCCTCCCCCCAGCCCCTCCTCCCCTTCCTGCACCCGTACCCCC |
| | GTGGTCTTTGAATAAAGTCTGAGTGGGCGGC |
| | (3′ UTR) |
| 62 | TGATAATAGGCTGGAGCCTCGGTGGCCATGCTTCTTGCCCCTT |
| | GGGCCCAAACACCATTGTCACACTCCATCCCCCCAGCCCCTCCT |
| | CCCCTTCCTCCATAAAGTAGGAAACACTACATGCACCCGTACCC |
| | CCGTGGTCTTTGAATAAAGTCTGAGTGGGCGGC |
| | (3′ UTR with miR-122 and miR-142-3p sites) |
| 63 | GSGATNFSLLKQAGDVEENPGP |
| | (2A peptide amino acid sequence) |
| 64 | GGAAGCGGAGCTACTAACTTCAGCCTGCTGAAGCAGGCTGGAGA |
| | CGTGGAGGAGAACCCTGGACCT |
| | (Nucleotide sequence encoding 2A peptide) |
| 65 | TCCGGACTCAGATCCGGGGATCTCAAAATTGTCGCTCCT |
| | GTCAAACAAACTCTTAACTTTGATTTACTCAAACTGGCTGGGGA |
| | TGTAGAAAGCAATCCAGGTCCACTC |
| | (Nucleotide sequence encoding 2A peptide) |
| 66 | GACAGUGCAGUCACCCAUAAAGUAGAAAGCACUACUAACAGCA |
| | CUGGAGGGUGUAGUGUUUCCUACUUUAUGGAUGAGUGUACUGUG |
| | (miR-142) |
| 67 | UGUAGUGUUUCCUACUUUAUGGA |
| | (miR-142-3p) |
| 68 | UCCAUAAAGUAGGAAACACUACA |
| | (miR-142-3p binding site) |
| 69 | CAUAAAGUAGAAAGCACUACU |
| | (miR-142-5p) |
| 70 | AGUAGUGCUUUCUACUUUAUG |
| | (miR-142-5p binding site) |
| 71 | CCUUAGCAGAGCUGUGGAGUGUGACAAUGGUGUUUGUGUCU |
| | AAACUAUCAAACGCCAUUAUCACACUAAAUAGCUACUGCUAGGC |
| | (miR-122) |
| 72 | AACGCCAUUAUCACACUAAAUA |
| | (miR-122-3p) |
| 73 | UAUUUAGUGUGAUAAUGGCGUU |
| | (miR-122-3p binding site) |
| 74 | UGGAGUGUGACAAUGGUGUUUG |
| | (miR-122-5p) |
| 75 | CAAACACCAUUGUCACACUCCA |
| | (miR-122-5p binding site) |
| 76 | GGGAAATAAGAGAGAAAAGAAGAGTAAGAAGAAATATAAGA |
| | GCCACC |
| | (Standard 5′ UTR sequence) |
| 77 | GGGAAATAAGAGAGAAAAGAAGAGTAAGAAGAAATATAAGA |
| | (5′ UTR sequence) |
| 78 | GGGAAATAAGAGAGAAAAGAAGAGTAAGAAGAAATATAAGA |
| | CCCCGGCGCCGCCACC |
| | (V1-UTR sequence) |
| 79 | GGGAAATAAGAGAGAAAAGAAGAGTAAGAAGAAATATAAGA |
| | CCCCGGCGCCACC |
| | (V2-UTR sequence) |
| 80 | CCCCGGCGCC |
| | (V1 GC-rich RNA element) |
| 81 | CCGCCGCCGCCG |
| | (GC-rich element wherein (CCG)ₙ, where n = 4) |
| 82 | CCGCCGCCGCCGCCG |
| | (GC-rich element wherein (CCG)ₙ, where n = 5) |

What is claimed:

1. An immune cell delivery lipid nanoparticle comprising:
(i) an ionizable lipid;
(ii) a sterol or other structural lipid;
(iii) a non-cationic helper lipid or phospholipid;
(iv) an agent for delivery to an immune cell, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to an immune cell.

2. The immune cell delivery lipid nanoparticle of claim 1, which comprises
a PEG-lipid, and wherein the agent comprises an mRNA encoding a protein of interest.

3. The immune cell delivery lipid nanoparticle of claim 1,
wherein one or more of (i) the ionizable lipid or (ii) the sterol or other structural lipid or (iii) the non-cationic helper lipid or phospholipid or (v) the PEG lipid is a C1q binding lipid that binds to C1q and/or promotes the binding of the LNP to C1q, as compared to a lipid nanoparticle lacking the C1q binding lipid.

4. The immune cell delivery lipid nanoparticle of claim 1, wherein the enhanced delivery is relative to a lipid nanoparticle lacking the immune cell delivery potentiating lipid.

5. The immune cell delivery lipid nanoparticle of claim 1, wherein the enhanced delivery is relative to a suitable control.

6. The immune cell delivery lipid nanoparticle of claim 1, wherein the agent stimulates protein expression in the immune cell.

7. The immune cell delivery lipid nanoparticle of claim 1, wherein the agent inhibits protein expression in the immune cell.

8. The immune cell delivery lipid nanoparticle of claim 1, wherein the agent encodes a soluble protein that modulates immune cell activity.

9. The immune cell delivery lipid nanoparticle of claim 1, wherein the agent encodes an intracellular protein that modulates immune cell activity.

10. The immune cell delivery lipid nanoparticle of claim 1, wherein the agent encodes a transmembrane protein that modulates immune cell activity.

11. The immune cell delivery lipid nanoparticle of claim 1, wherein the agent enhances immune function.

12. The immune cell delivery lipid nanoparticle of claim 1, wherein the agent inhibits immune function.

13. The immune cell delivery lipid nanoparticle of claim 1, wherein the immune cell is a T cell.

14. The immune cell delivery lipid nanoparticle of claim 1, wherein the immune cell is a B cell.

15. The immune cell delivery lipid nanoparticle of claim 1, wherein the immune cell is a dendritic cell or a myeloid cell.

16. The immune cell delivery lipid nanoparticle of claim 1, which comprises a phytosterol, or salt or ester thereof, or a combination of a phytosterol and cholesterol or a salt thereof.

17. The immune cell delivery lipid nanoparticle of claim 16, wherein the phytosterol is selected from the group consisting of β-sitosterol, stigmasterol, β-sitostanol, campesterol, brassicasterol, and combinations thereof.

18. The immune cell delivery lipid nanoparticle of claim 16, wherein the phytosterol comprises a sitosterol or a salt or an ester thereof.

19. The immune cell delivery lipid nanoparticle of claim 16, wherein the phytosterol comprises a stigmasterol or a salt or an ester thereof.

20. The immune cell delivery lipid nanoparticle of claim 16, wherein the phytosterol is beta-sitosterol 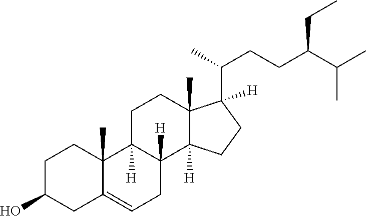
or a salt or an ester thereof.

21. (canceled)

22. The immune cell delivery lipid nanoparticle of claim 16, wherein the immune cell is a T cell and the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

23. The immune cell delivery lipid nanoparticle of claim 22, wherein the phytosterol is β-sitosterol.

24. The immune cell delivery lipid nanoparticle of claim 22, wherein the phytosterol is β-sitostanol.

25. The immune cell delivery lipid nanoparticle of claim 22, wherein the phytosterol is campesterol.

26. The immune cell delivery lipid nanoparticle of claim 22, wherein the phytosterol is brassicasterol.

27. The immune cell delivery lipid nanoparticle of claim 16, wherein the immune cell is a monocyte or a myeloid cell and the phytosterol or a salt or ester thereof is selected from the group consisting of β-sitosterol, and stigmasterol, and combinations thereof.

28. The immune cell delivery lipid nanoparticle of claim 27, wherein the phytosterol is β-sitosterol.

29. The immune cell delivery lipid nanoparticle of claim 27, wherein the phytosterol is stigmasterol.

30. The immune cell delivery lipid nanoparticle of claim 1, which comprises a sterol, or a salt or ester thereof, and cholesterol, wherein the immune cell is a monocyte or a myeloid cell and the sterol or a salt or ester thereof is selected from the group consisting of β-sitosterol-d7, brassicasterol, Compound S-30, Compound S-31 and Compound S-32.

31. The immune cell delivery lipid nanoparticle of claim 16, wherein the mol % cholesterol is between about 1% and 50% of the mol % of phytosterol present in the lipid nanoparticle.

32. The immune cell delivery lipid nanoparticle of claim 16, wherein the mol % cholesterol is between about 10% and 40% of the mol % of phytosterol present in the lipid nanoparticle.

33. The immune cell delivery lipid nanoparticle of claim 16, wherein the mol % cholesterol is between about 20% and 30% of the mol % of phytosterol present in the lipid nanoparticle.

34. The immune cell delivery lipid nanoparticle of claim 16, wherein the mol % cholesterol is about 30% of the mol % of phytosterol present in the lipid nanoparticle.

35. The immune cell delivery lipid nanoparticle of claim 1, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

36. The immune cell delivery lipid nanoparticle of claim 1, wherein the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M.

37. The immune cell delivery lipid nanoparticle of claim 1, wherein the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

38. (canceled)

39. The immune cell delivery lipid nanoparticle of claim 37, wherein immune cell is a T cell and the ionizable lipid comprises a compound selected from the group consisting of Compound I-301, Compound I-321, and Compound I-326.

40. The immune cell delivery lipid nanoparticle of claim 36, wherein immune cell is a monocyte or a myeloid cell and the ionizable lipid comprises a compound selected from the group consisting of Compound X, Compound I-109, Compound I-111, Compound I-181, Compound I-182, and Compound I-244.

41. The immune cell delivery lipid nanoparticle of claim 1, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.

42. The immune cell delivery lipid nanoparticle of claim 41, wherein the phospholipid is DSPC.

43. The immune cell delivery lipid nanoparticle of claim 41, wherein the immune cell is a T cell and the non-cationic helper lipid or phospholipid comprises a compound selected from the group consisting of DSPC, DMPE, and Compound H-409.

44.-46. (canceled)

47. The immune cell delivery lipid nanoparticle of claim 41, wherein the immune cell is a monocyte or a myeloid cell and the non-cationic helper lipid or phospholipid comprises a compound selected from the group consisting of DOPC, DMPE, and Compound H-409.

48.-50. (canceled)

51. The immune cell delivery lipid nanoparticle of claim 1, which comprises a PEG-lipid.

52. The immune cell delivery lipid nanoparticle of claim 51, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

53. The immune cell delivery lipid nanoparticle of claim 51, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

54. (canceled)

55. The immune cell delivery lipid nanoparticle of claim 51, wherein the PEG lipid comprises a compound selected from the group consisting of Compound P-428, Compound PL-16, Compound PL-17, Compound PL-18, Compound PL-19, Compound PL-1, and Compound PL-2.

56. The immune cell delivery lipid nanoparticle of claim 1, which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

57. The immune cell delivery lipid nanoparticle of claim 1, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

58. The immune cell delivery lipid nanoparticle of claim 1, which comprises about 50 mol % ionizable lipid, about 10 mol % non-cationic helper lipid or phospholipid, about 38.5 mol % sterol or other structural lipid, and about 1.5 mol % PEG lipid.

59. The immune cell delivery lipid nanoparticle of claim 56, wherein the mol % sterol or other structural lipid is 18.5% phytosterol and the total mol % structural lipid is 38.5%.

60. The immune cell delivery lipid nanoparticle of claim 56, wherein the mol % sterol or other structural lipid is 28.5% phytosterol and the total mol % structural lipid is 38.5%.

61. (canceled)

62. The immune cell delivery lipid nanoparticle of claim 1, which comprises:
(i) about 50 mol % ionizable lipid, wherein the ionizable lipid is a compound selected from the group consisting of Compound I-301, Compound I-321, and Compound I-326;
(ii) about 10 mol % phospholipid, wherein the phospholipid is DSPC;
(iii) about 38.5 mol % structural lipid, wherein the structural lipid is selected from β-sitosterol and cholesterol; and
(iv) about 1.5 mol % PEG lipid, wherein the PEG lipid is Compound P-428.

63. A method of delivering an agent to an immune cell, the method comprising contacting the immune cell with an immune cell delivery lipid nanoparticle comprising:
(i) an ionizable lipid;
(ii) a sterol or other structural lipid;
(iii) a non-cationic helper lipid or phospholipid;
(iv an agent for delivery to an immune cell, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to an immune cell,
such that the agent is delivered to the immune cell.

64. The method of claim 63, wherein the agent comprises a nucleic acid encoding the protein of interest,
such that expression of the protein of interest is induced in or on the immune cell.

65. The method of claim 63, comprising contacting a T cell with the immune cell delivery lipid nanoparticle,
wherein the agent comprises a nucleic acid that modulates T cell activation or activity,
such that T cell activation or activity is modulated.

66. A method of increasing an immune response to a protein, the method comprising contacting immune cells with an immune cell delivery lipid nanoparticle comprising:
(i) an ionizable lipid;
(ii) a sterol or other structural lipid;
(iii) a non-cationic helper lipid or phospholipid;
(iv) an agent comprising a nucleic acid molecule, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that the immune response to the protein is increased.

67. A method of increasing a T cell response to a cancer antigen, the method comprising contacting the T cell with an immune cell delivery lipid nanoparticle comprising:
(i) an ionizable lipid;
(ii) a sterol or other structural lipid;
(iii) a non-cationic helper lipid or phospholipid;
(iv) an agent comprising an mRNA encoding a chimeric antigen receptor (CAR) that binds the cancer antigen, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that the T cell response to the cancer antigen is increased.

68. The method of claim 66,
wherein the agent comprises an mRNA encoding an antigen of interest,
such that the immune response to the antigen of interest is enhanced in the subject, as compared to the immune response to the antigen of interest induced by an LNP encapsulating the mRNA encoding the antigen of interest but lacking the immune cell delivery potentiating lipid.

69. The method of claim 63, comprising contacting a B cell with the immune cell delivery lipid nanoparticle,
wherein the agent comprises a nucleic acid molecule that modulates B cell activation or activity,
such that B cell activation or activity is modulated.

70.-128. (canceled)
